(19) United States
(12) Patent Application Publication
(10) Pub. No.: US 2025/0345446 A1
(43) Pub. Date: Nov. 13, 2025

(54) BIFUNCTIONAL MOLECULES THAT SELECTIVELY INDUCE DEGRADATION OF EXTRACELLULAR TARGETS IN LYSOSOMES

(71) Applicant: Draupnir Bio ApS, Frederiksberg (DK)

(72) Inventors: Simon Glerup Pedersen, Frederiksberg (DK); Camilla Gustafsen, Frederiksberg (DK); Peder Søndergaard Madsen, Frederiksberg (DK); Joachim Pold Vilstrup, Frederiksberg (DK); Anna Quattropani, Frederiksberg (DK); Gavin Whitlock, Sandwich Kent (GB); Paul Alan Glossop, Sandwich Kent (GB); Simon Feldbaek Nielsen, Frederiksberg (DK)

(21) Appl. No.: 18/871,256

(22) PCT Filed: Jun. 23, 2023
(86) PCT No.: PCT/EP2023/067115

§ 371 Date: Dec. 3, 2024

(30) Foreign Application Priority Data
Jun. 23, 2022 (EP) 22180838.9
Dec. 28, 2022 (EP) 22216941.9

(51) Int. Cl. A61K 47/68; C07D 207/04; C07K 7/08; C07K 14/48; C07K 14/775; C07K 16/24; C07K 16/40

(52) U.S. Cl. CPC A61K 47/6811; C07D 207/04; C07K 7/083; C07K 14/48; C07K 14/775; C07K 16/241; C07K 16/40

(57) ABSTRACT

The present invention relates to bifunctional molecules which contain a protein-of-interest binding moiety linked through a linker group to a cellular receptor binding moiety preferably a moiety which binds to the receptor sortilin encoded by the gene SORT 1.

### TECHNICAL FIELD

The present invention relates to bifunctional molecules which contain a protein-of-interest binding moiety linked through a linker group to a cellular receptor binding moiety preferably a moiety which binds to the receptor sortilin encoded by the gene SORT1. Pharmaceutical compositions based on these bifunctional molecules represents an additional aspect of the present invention. These compounds and/or compositions may be used to treat disease states and conditions by removing secreted and/or transmembrane proteins through degradation inside of cells of a patient or subject in need of therapy. Methods of treating disease states and/or conditions in which circulation proteins are associated with the disease state and/or condition are also described herein.

### BACKGROUND

Protein degradation is an important part of turn-over and renewal of biomolecules and is a natural occurring process in all cells. Cytoplasmic proteins are commonly degraded in proteasomes following ubiquitination while extracellular biomolecules are degraded in lysosomal compartments. Specific ubiquitination of cytoplasmic disease associated proteins (DAP) is facilitated by bi-functional molecules termed PROteolysis TArgeting Chimeras (PROTACs) [1]. PROTACs are bifunctional molecules composed in one end of a DAP binding warhead, which is linked to E3 ubiquitin ligase binding small molecule at the other end. PROTACs thereby link the E3 ligase to DAPs, resulting in ubiquitination and subsequent degradation in the proteasome, effectively functioning as chemical DAP knockdown. However, as PROTACs rely on the proteasome for function it is only applicable for intracellular proteins. Data have shown that non-cytosolic proteins can be directed for degradation in lysosomal compartments by engaging the protein sorting mannose-6 phosphate receptor (M6P-R) using bi-functional molecules [2]. Banik et al. showed that antibodies tagged with a M6P-R binding sugar moiety facilitated lysosomal degradation of extracellular and membrane bound targets, and these types of molecules were termed LYsosomal TArgeting Chimeras (LYTACs), this provided first preclinical proof-of-concept (PoC) that enhancing lysosomal delivery and degradation of DAP's has therapeutic potential. The M6P-R binding motif is a phosphorylated sugar polymer, and substantial effort would therefore be required to develop an orally available modality. Both PROTACs and LYTACs are designed to hitchhike on natural cellular mechanisms, and as such the warheads are not required to provide functionality on their own. This is particular advantageous relative to traditional drug development, as warhead development simply requires optimisation of binding affinity and linker conjugation strategy. Additionally, the lack of these requirements further enables engagement against targets which are considered un-druggable due to current restraints.

The functionality of LYTACs rely on successful recruitment of a lysosomal transport receptor, exemplified by the M6P-R [2]. Another lysosomal receptor protein is sortilin. Sortilin possess a number of common features with the M6P-R including rapid internalisation from the cell surface and trafficking of cargo to lysosomes [3]. Sortilin is expressed in most tissues and facilitates lysosomal degradation of several known ligands [4] [5] [6]. The internalisation capacity of sortilin is illustrated by plasma accumulation of the natural ligand and frontotemporal lobar dementia (FTD) associated protein Progranulin, which is 3.5 fold increased in plasma of mice [6, 7] lacking sortilin. Increasing extracellular Progranulin levels by inhibition of sortilin mediated degradation of Progranulin is considered a therapeutic approach for treatment/prevention of FTD and several independent efforts have been explored to inhibit this interaction. The result is numerous small molecule high-affinity sortilin binders with different pharmacological profiles [8-10], including oral bioavailable compounds and compounds with CNS exposure [8].

### SUMMARY

In one main aspect, the present invention relates to bifunctional compounds having the structure according to formula (I):

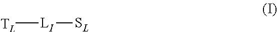

wherein,
S_{L} is a moiety that binds to Sortilin;
L_{I} is a linker or a bond; and
T_{L} is a moiety that binds an extracellular target molecule
or a pharmaceutically acceptable salt thereof.

In another aspect, the present invention relates to bifunctional compounds for use in the treatment of disorder or condition in a subject. In one aspect, the disorder or condition is mediated by an extracellular protein.

In yet another aspect, the present invention provides a method of targeted lysosomal degradation of an extracellular protein, said method comprising administering an effective amount of a bifunctional compound of formula (I) as described herein.

In another aspect, the present invention provides a method of removing an extracellular protein from the plasma of a subject in need thereof, the method comprising administering an effective amount of a bifunctional compound of formula (I) as described herein to said subject.

Thus, in one aspect the present disclosure provides for a method of targeted lysosomal degradation of TNFa, comprising administering an effective amount of the bifunctional compound as described herein.

In another aspect, the present disclosure provides for a method of removal of TNFa from the plasma of a patient or subject in need thereof, comprising administering a bifunctional compound as described herein.

In one aspect, the present disclosure relates to a pharmaceutical composition comprising a bifunctional compound as described herein.

In one aspect, the present invention relates to novel small molecule sortilin binders, as described herein.

In another aspect, the present invention relates to novel sortilin binding peptides, as described herein.

In another aspect, the present invention relates to an isolated polynucleotide encoding for the peptides or bifunctional compounds described herein. In another aspect, the present invention relates to a vector comprising a polynucleotide encoding for the peptides or bifunctional compounds described herein. In another aspect, the present invention relates to a host cell comprising a polynucleotide or a vector encoding for the peptides or bifunctional compounds described herein.

In another aspect, the present invention relates to novel TNFa binders, as described herein.

### DESCRIPTION OF DRAWINGS

FIG. 1: MST binding experiment using fluorescent Sortilin-6his (100 nM) and compounds according to SEQ ID NO.: 147 (A) and SEQ ID NO.: 157 (B).

FIG. 2: proximity induced HTRF ratio as function of compound concentration for compounds according to SEQ ID NO: 147 and SEQ ID NO.: 157.

FIG. 3: A) shows cell associated fluorescent intensity (FI) signal following 3 h incubation of HEK293/sortilin with a concentration series of SEQ ID NO.: 147, SEQ ID NO.: 157 or SEQ ID NO.: 155 and Neutravidin fluorescent derivative (NA650); B) cell associated FI signal following 3 h incubation of HEK293/sortilin with SEQ ID NO.: 147 (300 nM) and NA650 (Invitrogen) (100 nM) in presence of small molecule sortilin binder AF38496 (Schrøder et al) (0.1 nM-100 μM), SEQ ID NO.: 172 a peptide without biotin warhead (0.025 nM-25 μM), or biotin alone (0.1 nM-100 μM) (n=2). Data points are shown as mean value+/−SEM.

FIG. 4: A) fluorescence microscopy imaging with lysosomal marker in HEK293/sortilin cells fixed and immuno-stained with anti-LAMP1 (Alexa-488) following 2 h incubation with bifunctional molecule SEQ ID NO.: 147 (300 nM) and NA650 (100 nM). Nucleus stained with Hoechst. Scale bar is 20 μm. B) SDS-PAGE gel showing NA650 in cell lysates of HEK293/sortilin upon incubation with peptidic bifunctional compound (300 nM) and leupeptin (80 μM) (Sigma Aldrich) for 24 h prior harvest of cell-lysates and separation of proteins on SDS-PAGE gel. Bar graph shows quantification of NA650 upper band (mean value with std).

FIG. 5: NA650 fluorescence signal in HEK293/sortilin cell culture supernatant following incubation with SEQ ID NO.: 147 (20 nM to 10 μM) and NA650 (Invitrogen) (100 nM) for up to 24 h, 48 h and 72 h.

FIG. 6: NA650 fluorescence signal in HEK293/sortilin cell culture supernatant following incubation with NA650 (Invitrogen) (100 nM) and SEQ ID NO.: 147 (20 nM-5 μM) or SEQ ID NO.: 155 (20 nM to 5 M).

FIG. 7: A) SDS-PAGE analysis of conjugation of NHS-linker (SEQ ID NO.: 169) or NHS-linker-RQLL (SEQ ID NO.: 168) to Alirocumab under non-reduced or reduced (2 mM DTT+heating) conditions. B) SDS-PAGE analysis of conjugation of NHS-linker or NHS-linker-RQLL to Adalimumab under non-reduced or reduced (2 mM DTT+heating) conditions.

FIG. 8: shows MST binding to sortilin response as function of antibody concentration for Alirocumab-link-RQLL and Alirocumab-link.

FIG. 9: shows that complex formation between PCSK9-6HIS and alirocumab-linker-RQLL (FIG. 9A) or alirocumab-linker (FIG. 9B) after purification by gel filtration. SDS-PAGE analysis of peak fractions from the protein complex (FIG. 9C). 5 FIG. 10: Baseline corrected HTRF (665/620 nm) A) ratio normalised to alirucomab-Link plotted against a concentration gradient of alirocumab conjugated with linker and with or without RQLL (290 nM-3.5 fM) following 2.5 hrs incubation. (Error bars=S.D.; n=2); B) corrected to the lowest global signal plotted against a concentration gradient of adalimumab conjugated with linker and with or without RQLL (400 nM-4.7 fM) after 2.5 hrs incubation (Error bars=S.D.; n=3). C) corrected to the lowest global signal plotted against a concentration gradient of adalimumab conjugated with sortilin binding peptides SEQ ID NO.: 184 to SEQ ID NO.: 187 (400 nM-4.7 fM) after 2.5 hrs incubation. (Error bars=S.D.; n=3).

FIG. 11: cell associated fluorescent signal A) following 3 h incubation of HEK293/sortilin with: A) a concentration series of Alirocumab-link-RQLL (SEQ ID NO.: 159) (1 nM to 0.5 μM) and Cy5-PCSK9 (200 nM). In a control experiment, cells were incubated with Alirocumab with a peptide lacking the C-terminal sortilin binding sequence Alirocumab-link (SEQ ID NO.: 160); B) a concentration series of adalimumab-link-RQLL (SEQ ID NO.: 161) (250 pM to 250 nM) and TNFalpha (100 nM). In a control experiment, cells were incubated with Adalimumab with a peptide lacking the C-terminal sortilin binding sequence adalimumab-link (SEQ ID NO.: 162) and C) a concentration series of adalimumab conjugated with sotilin binding peptides of SEQ ID NO.: 184 to SEQ ID NO.: 187 (250 pM to 250 nM) and TNFalpha (100 nM).

FIG. 12: shows the size-exclusion chromatography elution profile and final sample of A) NB-linker-RQLL, SEQ ID NO.: 158 and B) NB-linker, SEQ ID NO.: 71. Pooled and concentrated fractions from size-exclusion are indicated with a grey box.

FIG. 13: shows that complex formation between IgG target and (FIG. 13A) NB-linker-RQLL, SEQ ID NO.: 158 or (FIG. 13B) NB-linker, SEQ ID NO.: 71 was formed and could be purified by gel-filtration. The SDS-PAGE analysis of the main peak is shown on the right, pooled and concentrated fractions from size-exclusion are indicated with a grey box.

FIG. 14: A) shows cell associated FI signal following 3 h incubation of HEK293/sortilin with a concentration series of NB-linker-RQLL, SEQ ID NO.: 158 (8 nM to 8 μM) and Cy5 conjugated IgG (5, 50 or 500 nM); B) Cy5 FI in cell culture supernatant (upper blot) and lysates (middle blot) of HEK293/sortilin cells following 72 h incubation with Cy5 conjugated IgG (kappa-light chain) (50 nM) and NB-linker-RQLL, SEQ ID NO.: 158 as indicated. Lower blot confirms sortilin expression in cells by Western blotting. C) Cy5 FI (upper blot) and Western blot of IgG signal in lysates of HEK293/sortilin cells following 6:30 h incubation with Cy5 conjugated IgG (kappa-light chain) (50 nM), NB-linker-RQLL, SEQ ID NO.: 158 or control nanobody (NB-linker, SEQ ID NO.: 71) (250 nM), and lysosomal protease inhibitor leupeptin (80 μM) as indicated (n=2). The two lower blots confirm loading of gel (anti-β-actin) and sortilin expression in cells by Western blotting. Bar graph shows quantification of Ig HC as mean value+/−SEM.

FIG. 15. Detection of binding by titrated BF025, BF023 and BF020 (Top concentration 12.5 μM) with constant NTA labeled Sortilin-6His (100 nM) in MST analysis. (Error bars=S.D.; n=2)

FIG. 16. HTRF (665/620 nm) signal baseline corrected to the lowest local signal plotted against bifunctional compound concentration (5 μM-0.6 pM) after 2.5 hrs incubation.

FIG. 17. shows cell associated FI signal following 3 h incubation of HEK293/sortilin with a concentration series (2 nM to 2 μM) of compounds B025, B023, or B020 together with NA650 (100 nM).

FIG. 18. NA650 FI signal in HEK293/sortilin cell culture supernatant following incubation with NA650 (100 nM) together with bifunctional compounds s BF025, BF023 or BF20 (20 nM to 5 μM) as indicated for 72 h.

FIG. 19. FI signal in lysate of HEK293/sortilin harvested after 24 h incubation with NA650 (100 nM), B025 (300 nM), AF38469 (10 μM) and leupeptin (80 μM) as indicated.

FIG. 20. Cell associated FI signal following 3 h incubation of HEK293/sortilin with a concentration series (1.9 nM to 2 M) of compounds BF011, BF006, or BF005 and AlexaFluor488-anti-DNP antibody (100 nM) (n=2).

FIG. 21. A) FI signal in lysates of HEK293/sortilin incubated 3 h with AlexaFluor488-anti-DNP antibody (Thermo Fisher) (100 nM) and BF005 (30 nM) before removal of target and degrader by replacement of medium with assay medium and incubated for 0 h to 24 h before cells were lysed as described previously. Anti-β-actin and anti-sortilin Western blots are shown as control. Bar graph shows quantification of FI signal of the HC band normalized to β-actin signal (mean value+/−SEM) (n=2). B) FI in lysates of HEK293/sortilin incubated 6: 30 h with AlexaFluor488-anti-DNP antibody (100 nM), BF005 (30 nM) and leupeptin (80 M) as indicated. SB001 (30 nM) and AF38469 (10 μM) were included as control. Bar graph shows quantification of FI signal of the HC band normalized to β-actin signal (mean value+/−SEM) (n=2).

FIG. 22. cell associated FI signal following 3 h incubation of HEK293 or HEK293/sortilin with a concentration series (2 nM to 2 μM) of three different bifunctional compounds: SEQ ID NO.: 155, NB-link-RQLL (SEQ ID NO.: 158), or small molecule bifunctional compound (BF005) and corresponding targets as indicated.

FIG. 23 is a scheme representing a non-limiting example for the preparation of bifunctional compounds.

FIG. 24. A) shows detection of binding by titrated BF080, BF081 and BF082 (top concentration 12.5 μM) with constant NTA labeled TNFa-6His (100 nM) in MST analysis. (Error bars=S.D.; n=4-6). B) mouse anti-TNFalpha (Invitrogen, MA5-23720) Western blot of cell lysates following 24 h incubation of HEK393/sortilin with a concentration series (100 nM-10 μM) of compounds BF040 and BF043 together with TNFalpha (100 nM). Control cells were incubated without addition of compounds and TNFalpha or with TNFalpha alone. Anti-β-actin (Sigma, A5441) Western blot is shown as control. C) cell associated FI signal following 24 h incubation of HEK293/sortilin with a concentration series (0.5 nM to 20 μM) of BF040,BF043 and BF042 and Cy5-TNFalpha (100 nM) in the presence of 80 M leupeptin. D) cell associated FI signal following 24 h incubation of HEK293/sortilin cells with BF042 (0.5 nM to 20 μM) and Cy5-TNFalpha with or without addition of leupeptin (80 μM). E) shows TNFalpha levels in conditioned media of HEK293/sortilin cells 72 h after addition of 20 nM TNFalpha and bifunctional molecule BF040 (BF043) or BF042. TNFalpha is shown in % normalised to level measured in conditioned media from cells without addition of bifunctional molecule. F) anti-TNFalpha Western blotting of HEK293/sortilin cell lysates harvested at indicated timepoints (0 h to 24 h) following 24 h pre-incubation with BF042 (3 μM) and TNFalpha (100 nM). Pre-incubation media was replaced at timepoint 0 h. Anti-beta-actin Western blotting is shown as control. G) cell associated FI signal following 24 h incubation of HEK293/sortilin with a concentration series (0.5 nM to 20 μM) of BF042 and Cy5-TNFalpha (100 nM) with addition of sortilin binder SB013 (1.25, 5.0 and 10 μM) or DMSO. H) shows cell associated FI signal following 24 h incubation of HEK293/sortilin with or BF077 (1 M) and Cy5-TNFalpha (100 nM) in the presence of increasing concentrations of TF018 or TF005 as competitor of TNFalpha binding.

FIG. 25 illustrates efficacy of a TNFa degrader in an in vivo model of LPS induced acute systemic inflammation.

### DEFINITIONS

The term “alkyl” as used herein refers to a linear or branched hydrocarbon moiety.

The term “alkoxy” as used herein refers to a group of formula-O-alkyl, wherein alkyl is defined as above. In particular, C₁-C₃-alkoxy is intended to indicate such hydrocarbon having 1, 2 or 3 carbon atoms. Examples of alkoxy groups include methoxy, ethoxy, n-propoxy, and isopropoxy.

The term “haloalkyl” as used herein refers to an alkyl group wherein one or more hydrogen atoms have been replaced by a halogen atom, for example one or more hydrogen atoms replace by any of F, Cl, Br or I.

As used herein the term “cycloalkyl” or “carbocycle” refers to a monocylcic or polycyclic system. The term “cycloalkyl” also used herein can optionally contain one or more unsaturations or substituents.

The term “heterocyclic” or “heterocycle” as used herein, alone or in combination, refers to saturated or unsaturated aromatic or nonaromatic rings containing from 3 to 7 ring atoms where at least one the ring atoms are heteroatom(s). The term “heteroaromatic” or “heteroaryl” as used herein, alone or in combination, refers to an aromatic ring containing from 5 to 6 ring atoms where at least one of the ring atoms are heteroatom(s). By “heteroatom” is intended to mean sulfur, oxygen or nitrogen.

The term “aromatic” or “aryl” refers to a cyclic or polycyclic moiety having a conjugated unsaturated (4η+2)π electron system (where n is a positive integer), sometimes referred to as a delocalized IT electron system.

The term “alkenyl” embraces radicals having at least one carbon-carbon double bond.

The terms “substituents” or “substituted” as used herein, alone or in combination, refer to groups which may be used to replace hydrogen. The substituted molecule may itself be further substituted in some embodiments of the invention. As referred here a “substituent derived from” refer to a group of atoms derived from a specific molecule or formula at any position of said molecule or formula. In some embodiments, a substituent derived from a molecule is the corresponding molecule wherein a hydrogen atom has been removed. For example, a substituent derived from CH₄ may be —CH₃.

The dissociation constant (K_{D}) or binding affinity is a measure of the extent of a reversible association between two molecular species. The smaller the dissociation constant, the stronger the affinity of binding.

As describe herein, a ternary complex is a complex containing three different molecules that are bound together. As described herein, the bifunctional compounds are able to form ternary complexes between sortilin and the target molecules. This means, a three member complex where sortilin is bound to the bifunctional compound at the same time as the target protein.

An extracellular molecule or protein, as described herein refers to molecules or proteins that are not fully enclosed inside of a cell. This means for example, a protein that is completely outside of a cell, but also membrane-bound or membrane associated proteins with an extracellular domain.

As used herein, TNF-alpha may be referred to as TNFa, TNF-α, TNF-α, TNFα, or TNFalpha.

### DETAILED DESCRIPTION

In one main aspect, the present invention relates to bifunctional compounds having the structure according to formula (I):

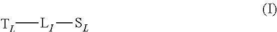

wherein,
S_{L} is a moiety that binds to Sortilin;
L_{I} is a linker or a bond; and
T_{L} is a moiety that binds an extracellular target molecule
or a pharmaceutically acceptable salt thereof.

Compounds of formula (I) function to bind an extracellular target molecule or protein of interest to Sortilin in a ternary complex. This recruits the target molecule or protein into the cell's lysosomal pathways leading to degradation of the target molecule or protein. Thus, the present invention relates to a platform of chemical entities able to perform targeted degradation of extracellular disease targets by engaging the sortilin receptor.

The advantages of the present invention are for example:
Ability to induce targeted degradation of extracellular disease proteins.
Ability to target a broad disease space due to wide range of body compartments where there is expression of sortilin: blood stream, CNS, PNS, CSF, cells of the immune system and tumor subtypes.

The inventors have shown the ability to construct such a platform by producing bifunctional compounds according to formula (I) in a plurality of forms: from small molecules to large proteins, passing through intermediate sized peptides.

### Binding of Sortilin:

Sortilin (SEQ ID NO.: 1) is a membrane protein expressed in most tissues that facilitates lysosomal degradation of several known ligands [4] [5] [6]. Structural studies from several independent researchers have shown that sortilin interacts with ligands inside the cavity of a large 10-bladed β-propeller [10, 11], and that cargo is released following maturation and acidification of endosomal vesicle leading to lysosomal degradation of cargo and recycling of sortilin back to the plasma membrane [12]. Altogether, sortilin a well described lysosomal trafficking receptor with multiple different high affinity small molecule binding partners

The present disclosure relates to a new platform of bifunctional compounds able to target sortilin to recruit target molecules or proteins and internalize them into the lysosomal compartments. Therein, producing their degradation under the lysosomal conditions. Thus, numerous and diverse different binders of sortilin can be employed.

Due to the potential of targeting sortilin inhibition as a therapeutic approach for conditions, numerous disclosures of sortilin binders exist in the art. These are based on small molecule scaffolds, as well as on peptides and proteins.

### Small Molecules

For instance, several small molecule scaffolds have been used to develop compounds with high affinity for sortilin. The first small molecule sortilin binder identified was compound AF40431 (Andersen et al. 2013 [14]). Thereafter, several promising scaffolds for developing sortilin binders have been proposed, such as formula IV in WO 2014/114779, formulas II and VI in Stachel et al. 2020 or formula VII in Andersent et al. 2017 [9]. Other scaffolds able to bind sortilin are disclosed in US 2016/0331646, such as scaffolds based on norbornene anhydride amino acid adducts (formulas VIII and IX), phenyl-amide-acids of benzyl substituted glutaric acids (Formula X), and 2-substituted 3-oxo-1,2,3,4-tetrahydro-2-quinoxalines (Formula XI). Other examples of reported sortilin binding compounds are SB013 and SB014, disclosed in Sparks et al. 2020. The content of the above mentioned references with protein scaffolds are hereby incorporated by reference.

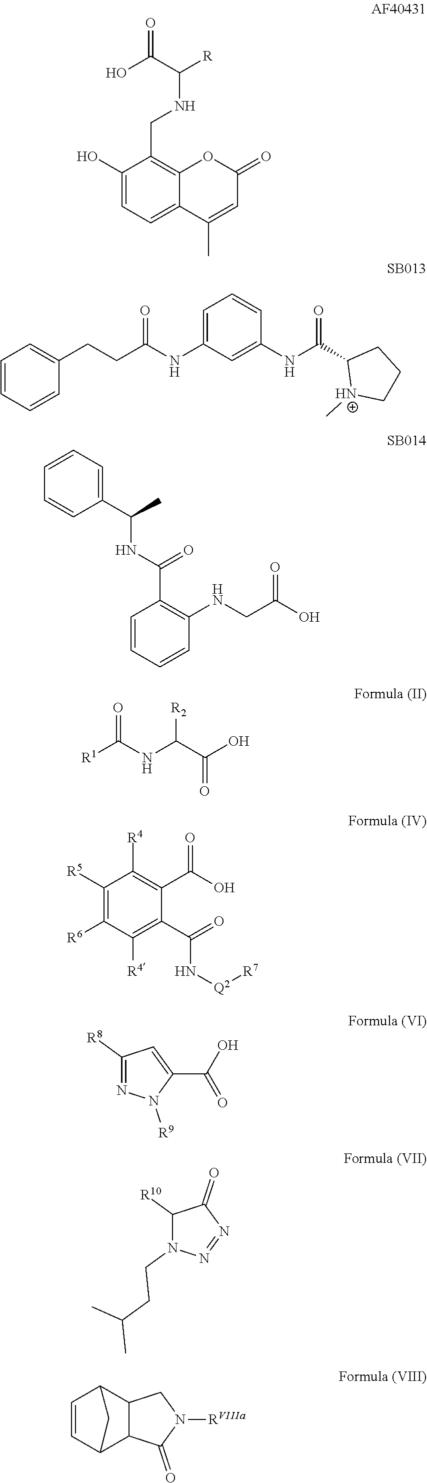

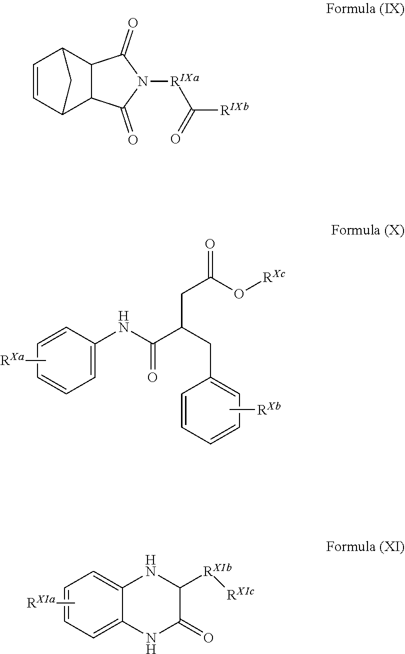

Thus, in one embodiment, the bifunctional compounds according to the present are able to bind sortilin through substituents derived from the above structures or derivatives thereof.

In one embodiment, the sortilin binding moiety (S_{L}) in the bifunctional compound according to formula (I) has a structure according to formula (II):

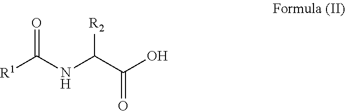

wherein
R¹ is selected from the group consisting of heteroaryl, aryl, heterocycle, C₃-C₁₀ cycloalkyl, —(C₁-C₅ alkyl)-aryl and C₁-C₁₀ alkyl, —B—O-aryl, each of which is optionally substituted with one or more, identical or different, substituents R^{IIa}; wherein B is an optionally substituted C₁-C₅ alkyl;
R² is selected from the group consisting of C₁-C₁₀ alkyl and C₂-C₁₀ alkenyl, each of which is optionally substituted with one or more, identical or different, substituents R^{IIb};
R^{IIa} is selected from the group consisting of —O-aryl, —CH₂-aryl, Cl, Br, F, C₁-C₃ alkoxy and C₁-C₃ alkyl, wherein each of —O-aryl, —CH₂-aryl, C₁-C₃ alkoxy and C₁-C₃ alkyl is optionally substituted with one or more, identical or different substituents selected from the group consisting of H, —O—CH₂—C(═O)O—, —O—CH₂—C(═O)NH—, halogen, alkoxy, —CF₃, and an optionally substituted C₁-C₄ alkyl;
R^{IIb} is selected from the group consisting of C₁-C₁₀ alkoxy, C₃-C₁₀ cycloalkyl, C₁-C₁₀ alkyl and —CH₂-aryl;
wherein S_{L} is conjugated to L_{I} via R¹ or R^{IIa}.

In one embodiment, R¹ has two identical R^{IIa} substituents as described above. In one embodiment, R¹ has two different R^{IIa} substituents as described above. In one embodiment, R¹ has only one R^{IIa} substituent as described above.

In one embodiment, R¹ is an optionally substituted phenyl group. In one embodiment, R¹ is a phenyl group with one or two substituents selected from the group consisting of halogen or alkoxy. In one embodiment, R¹ is a phenyl group substituted with two chlorine atoms. In a further embodiment, R¹ is phenyl group substituted with two chlorine atoms situated in the meta-positions from the point of attachment with formula II. In one embodiment, R¹ is a phenyl group with one alkoxy substituent. In a further embodiment, the alkoxy substituent is —OCH₃. In one embodiment, R¹ is according to formula B:

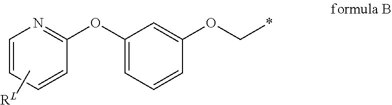

wherein R^{L} denotes the attachment to L_{I} and * denotes the attachment to formula II.

In one embodiment, R² is a C₁-C₆ alkyl. In one embodiment, R² is a substituted C₁ alkyl, wherein the C₁ alkyl is substituted with an optionally substituted C₃-C₆ cycloalkyl. In one embodiment, R² is a substituted C₁ alkyl, wherein the C₁ alkyl is substituted with a C₆ cycloalkyl. In one embodiment, R² is tert-butyl. In one embodiment, R² is —CH₂—C—(CH₃)₃.

In one embodiment, the sortilin binding moiety (S_{L}) in the bifunctional compound according to formula (I) has a structure according to formula (III):

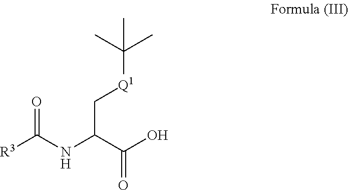

wherein Q¹ is a bond or —CH₂—; and
R³ is of Formula (IIIa), Formula (IIIb) or formula B;

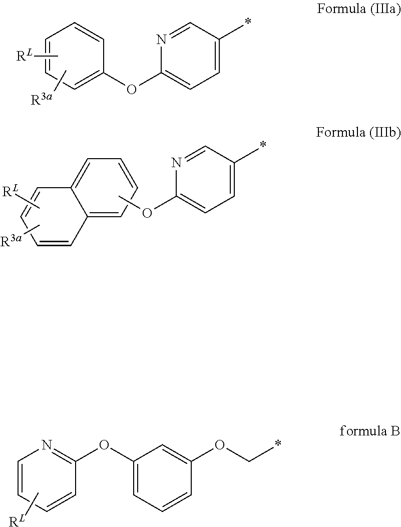

wherein
R^{3a} is selected from the group consisting of H, halogen, alkoxy, —CF₃, and an optionally substituted C₁-C₄ alkyl;
wherein * denotes the attachment with Formula (III) and R^{L} denotes the attachment with L_{I}.

As described herein, R_{L} denotes the point of attachment between L_{I} and S_{L} or the point of attachment between T_{L} and L_{I}.

In some embodiments, S_{L} is according to any one selected from the group consisting of formulas IIIc, IIId, IIIe, IIIf, IIIg and IIIh:

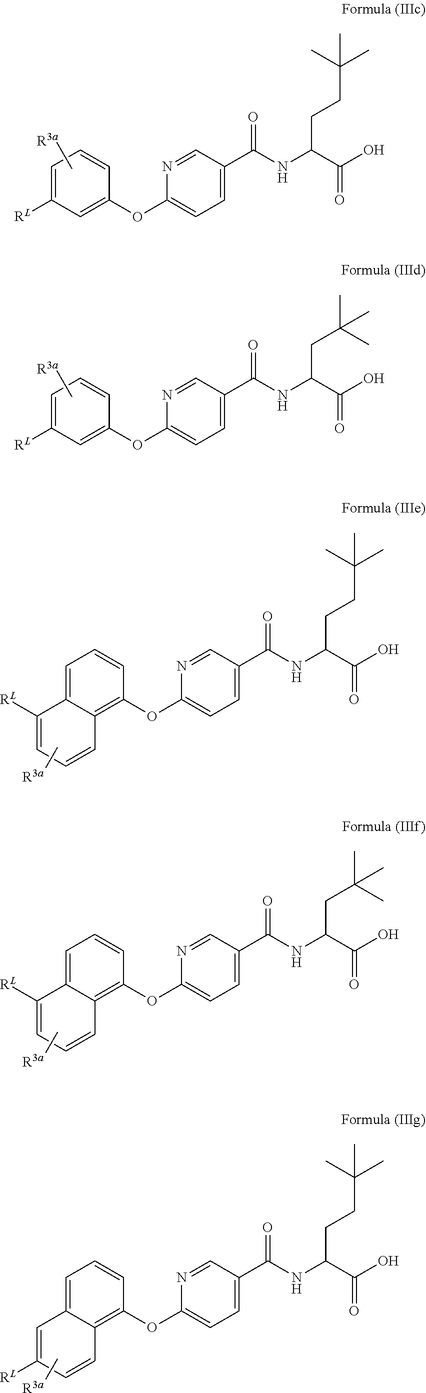

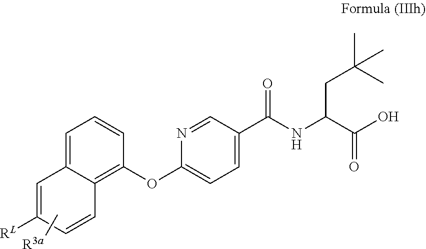

wherein R^{3a} is selected from the group consisting of H, halogen, alkoxy, —CF₃, and an optionally substituted C₁-₄ alkyl; and
R^{L} denotes the attachment with L_{I}.

In one embodiment, the bifunctional compound according to the present disclosure has S_{L} according to formula IIIc. In one embodiment, the bifunctional compound according to the present disclosure has S_{L} according to formula IIId. In one embodiment, the bifunctional compound according to the present disclosure has S_{L} according to formula IIIe. In one embodiment, the bifunctional compound according to the present disclosure has S_{L} according to formula IIIf. In one embodiment, the bifunctional compound according to the present disclosure has S_{L} according to formula IIIg. In one embodiment, the bifunctional compound according to the present disclosure has S_{L} according to formula IIIh. In formulas each of IIIc, IIId, IIIe, IIIf, IIIg and IIIh R^{3a} is selected from the group consisting of H, halogen, alkoxy, —CF₃, and an optionally substituted C₁-₄ alkyl; and R^{L} denotes attachment to L^{I} as described in formula (I).

In one embodiment, R^{3a} is H. In another embodiment, R^{3a} is halogen or —CF₃. In one embodiment, R^{3a} is alkoxy In yet another embodiment, R^{3a} is an optionally substituted C₁-₄ alkyl.

In some embodiments, S_{L} has a structure according to any one of formulas IIIi, IIIj, IIIk, IIIm, IIIn, and IIIo:

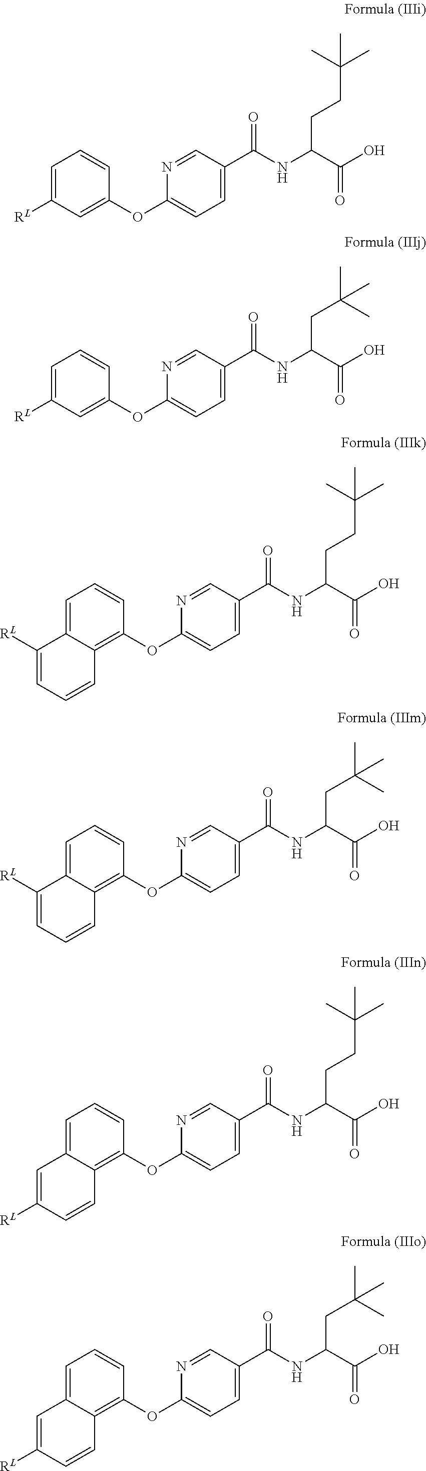

R^{L} denotes the attachment with L_{I}.

In one embodiment, the bifunctional compound according to the present disclosure has S_{L} according to formula IIIi. In one embodiment, the bifunctional compound according to the present disclosure has S_{L} according to formula IIIj. In one embodiment, the bifunctional compound according to the present disclosure has S_{L} according to formula IIIk. In one embodiment, the bifunctional compound according to the present disclosure has S_{L} according to formula IIIm. In one embodiment, the bifunctional compound according to the present disclosure has S_{L} according to formula IIIn. In one embodiment, the bifunctional compound according to the present disclosure has S_{L} according to formula IIIo. In formulas IIIi, IIIj, IIIk, IIIm, IIIn and IIIo R^{L} denotes attachment to L^{I} as described din formula (I).

In another embodiment, the sortilin binding moiety (S_{L}) in the bifunctional compound according to formula (I) has a structure according to formula (D-I):

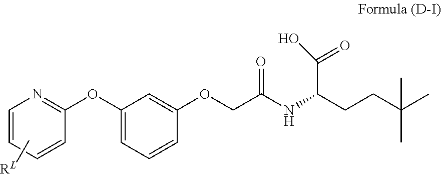

wherein R^{L} denotes attachment to L_{I}.

In another embodiment, the sortilin binding moiety (S_{L}) in the bifunctional compound according to formula (I) has a structure according to formula (D-II):

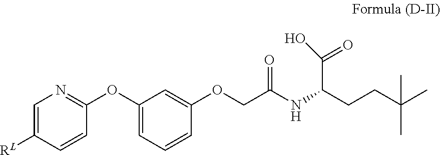

wherein R^{L} denotes attachment to L_{I}.

The structures according to formulas III and IIIa to IIIo as described herein possess a stereocentre at the α-position of the carboxylic acid group, as marked below by “a” in formula III:

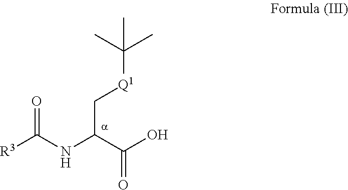

A stereocentre can exist in one of two configurations: R or S. These formulas III and IIIa to IIIo exist in either the S-stereoisomer configuration or R-stereoisomer configuration.

Thus, in one embodiment S_{L} is according to the S-stereoisomer of any one of formulas III or IIIa to IIIo as described herein.

In another embodiment, S_{L} is according to the R-stereoisomer of any one of formulas III or IIIa to IIIo as described herein.

In another embodiment, the sortilin binding moiety (S_{L}) in the bifunctional compound according to formula (I) has a structure according to formula (IV):

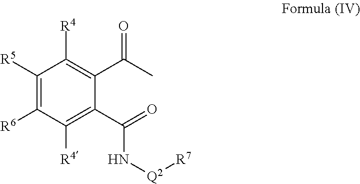

R⁴ is H or F;
R^{4′} is H;
R⁵ is halogen, H, C₁-C₆ alkyl, C₂-₆ alkenyl or C₁-C₆ haloalkyl;
R⁶ is halogen, H, C₁-C₆ alkyl or C₁-C₆ haloalkyl;
Q² is a bond or CH₂;
R⁷ is a 5-6 membered heteroaromatic monocyclic ring with one or two heteroatom(s), wherein the heteroaromatic ring is optionally substituted with one or two substituents individually selected from the group consisting of —CN; C₁-C₃ alkyl; halogenated C₁-C₃ alkyl; C₁-C₃alkoxy, halogen; —C(O)NH—C₁-C₃ alkyl; aryl optionally substituted with —C(O)NH—C₁-C₃ alkyl; and optionally substituted heteroaryl,
or a pharmaceutically acceptable salt thereof, wherein S_{L} is conjugated to L_{I} via R⁷.

In one embodiment, R⁵ is halogen, H, or C₁-C₆ haloalkyl; and R⁶ is halogen, H, C₁-C₆ alkyl or C₁-C₆ haloalkyl.

In a another embodiment, R⁴ is H, R⁵ is —CF₃ or H; R⁶ is —CF₃ or H; and R^{4′} is H.

In one embodiment, Q² is a bond. In another embodiment, Q² is —CH₂—.

In one embodiment, R⁷ is a 6 membered heteroaromatic monocyclic ring with one heteroatom substituted with a C₁ alkyl, wherein said heteroaromatic ring is optionally further substituted with an optionally substituted aryl, and S_{L} is conjugated to L_{I} via R⁷.

In one embodiment, Q² is a bond, R⁴ is H, R⁵ is —CF₃ or H; R⁶ is —CF₃ or H; R^{4′} is H, Q² is a bond, R⁷ is a 6 membered heteroaromatic monocyclic ring with one heteroatom substituted with a C₁ alkyl, wherein said heteroaromatic ring is optionally further substituted with an optionally substituted aryl, and wherein S_{L} is conjugated to L_{I} via R⁷.

In one embodiment, at least one of R⁵ or R⁶ is —CF₃. In one embodiment, at least one of R⁵ or R⁶ is —CF₃ while the other one is hydrogen.

In one embodiment, R⁷ has a structure according to formula IVa or formula IVb:

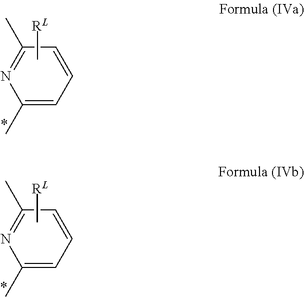

wherein * denotes attachment to formula (IV) and wherein R^{L} denotes the attachment to L_{I}.

In another embodiment, S_{L} is selected from any one of the group consisting of formulas IVc, IVd, IVe and IVf:

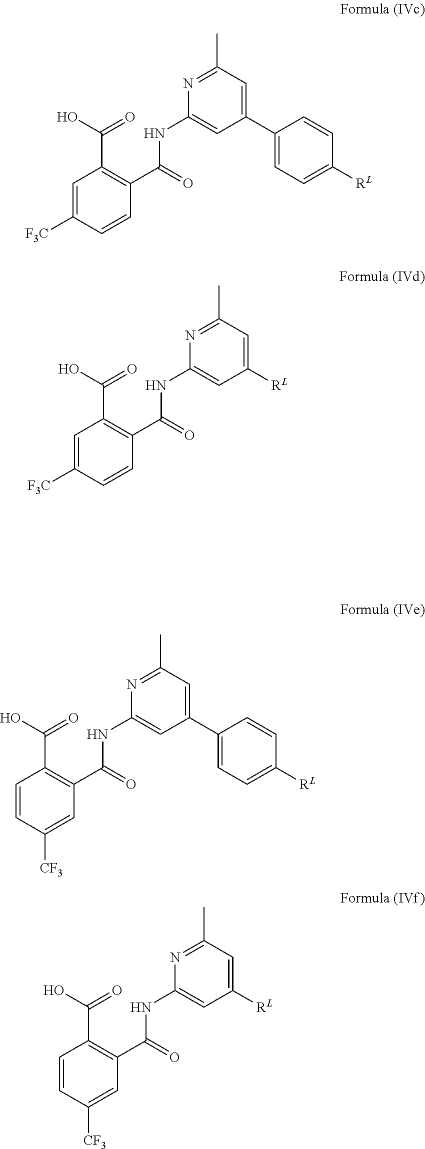

wherein R^{L} denotes the attachment to L_{I}.

In one embodiment, the bifunctional compound according to the present disclosure has S_{L} according to formula IVc. In one embodiment, the bifunctional compound according to the present disclosure has S_{L} according to formula IVd. In one embodiment, the bifunctional compound according to the present disclosure has S_{L} according to formula IVe. In one embodiment, the bifunctional compound according to the present disclosure has S_{L} according to formula IVf. In formulas IVc, IVd, IVe and IVf R_{L} denotes the attachment to L_{I}.

In another embodiment, the sortilin binding moiety (S_{L}) in the bifunctional compound according to formula (I) is a substituent derived from a compound of formula VI:

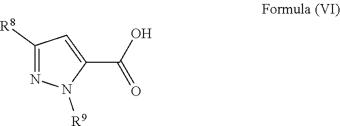

wherein
R⁸ is C₁-C₁₀ alkyl; and
R⁹ is selected from the group consisting of C₁-C₁₀ alkyl, aryl, and heteroaryl, each of which is optionally substituted with one or more, identical or different, substituents R^{VIa};
R^{VIa} is selected from the group consisting of aryl, heteroaryl, —O-aryl, —O-heteroaryl and halogen, wherein each of aryl, heteroaryl, —O-aryl and —O-heteroaryl is optionally substituted with one or more halogen(s).

In one embodiment, R⁸ is a C₁-C₅ alkyl. In one embodiment, R⁸ is tert-butyl. In another embodiment, R⁸—CH₂—C—(CH₃)₃.

In one embodiment, R⁹ is an optionally substituted aryl. In one embodiment, R⁹ is an optionally substituted benzyl. In one embodiment, R⁹ is —C₁-C₃-aryl.

In one embodiment, R⁹ is one selected from the group consisting of:

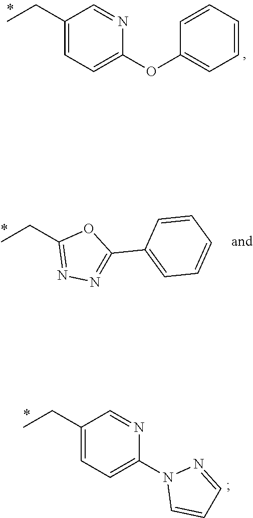

wherein * denotes the attachment with formula VI.

In one embodiment R⁹ is substituted with one or two substituents R^{VIa}. In a further embodiment, the one or two substituents R^{VIa} are halogen.

In one embodiment, formula VI is connected to the linker moiety (L_{I}) in formula (I) via R⁹.

In another embodiment, the sortilin binding moiety (S_{L}) in the bifunctional compound according to formula (I) is a substituent derived from a compound of formula VI:

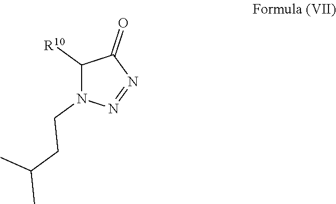

wherein
R¹⁰ is

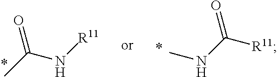

and
R¹¹ is an optionally substituted heteroaryl or an optionally substituted aryl.

In one embodiment, R¹¹ is a pyridyl group. In one embodiment, formula VII is connected to the linker moiety (L_{I}) in formula (I) via R¹¹.

In one embodiment, the sortilin binding moiety (S_{L}) in the bifunctional compound according to formula (I) is a substituent derived from the compound:

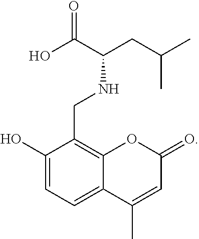 [SMILES: Cc1cc(=O)oc2c(CN[C@@H](CC(C)C)C(=O)O)c(O)ccc12]

In one embodiment, the sortilin binding moiety (S_{L}) in the bifunctional compound according to formula (I) is a substituent derived from the compound:

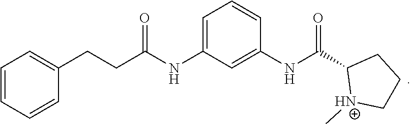 [SMILES: C[NH+]1CCC[C@H]1C(=O)Nc1cccc(NC(=O)CCc2ccccc2)c1]

In one embodiment, the sortilin binding moiety (S_{L}) in the bifunctional compound according to formula (I) is a substituent derived from the compound:

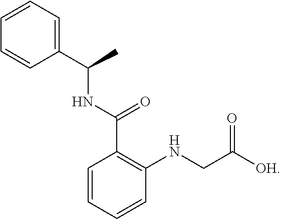 [SMILES: C[C@@H](NC(=O)c1ccccc1NCC(=O)O)c1ccccc1]

In another embodiment, the sortilin binding moiety (S_{L}) in the bifunctional compound according to formula (I) is a substituent derived from a compound of formula VIII or formula IX:

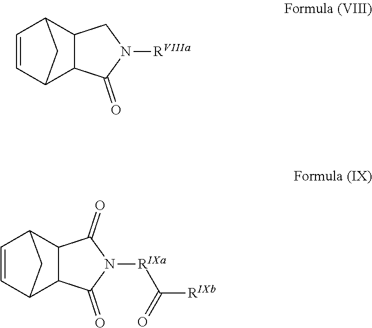

wherein,
R^{VIIIa} is a carboxylic acid or ester thereof and where any of the carbons of the norbornene ring can be substituted with a C₁₋₆ alkyl group or the carboxylic acid can be condensed as a C₁₋₅ ester.
wherein R^{IXa} is an optionally substituted bivalent C₁-C₅ alkyl, wherein one or more methylene groups is optionally replaced by —CO(NH)—,
R^{IXb} is a group selected from the group consisting of hydroxyl, alkoxy, amino or aminoalkyl.

In one embodiment, the sortilin binding moiety (S_{L}) in the bifunctional compound according to formula (I) is a substituent derived from the compound 2-methyl-3,5-dioxo-4-azatricyclo[5.2.1.0 (2,6)]dec-8-en-4-yl) acetic acid.

In one embodiment, the sortilin binding moiety (S_{L}) in the bifunctional compound according to formula (I) is a substituent derived from the compound methyl 2-(1,3-dioxo-1,3,3a,4,7,7a-hexahydro-2H-4,7-methanoisoindol-2-yl) propanoate.

In one embodiment, the sortilin binding moiety (S_{L}) in the bifunctional compound according to formula (I) is a substituent derived from the compound 2-(1,3-dioxo-1,3,3a,4,7,7a-hexahydro-2H-4,7-methanoisoindol-2-yl)-4-(methylthio) butanoic acid

In one embodiment, the sortilin binding moiety (S_{L}) in the bifunctional compound according to formula (I) is a substituent derived from the compound 2-(3,5-dioxo-4-azatricyclo[5.2.1.0 (2,6)]dec-8-en-4-yl)-4-methylpentanoic acid.

In another embodiment, the sortilin binding moiety (S_{L}) in the bifunctional compound according to formula (I) is a substituent derived from a compound of formula X:

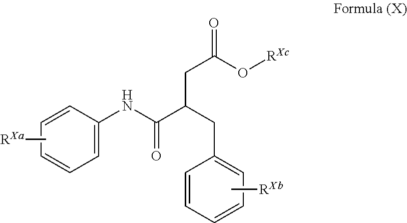

wherein
R^{Xa} and R^{Xb} are independently C₁-C₅ alkyl, acyl, amino, sulfono, chloro, bromo, iodo, or flouro; and
R^{Xc} is C₁-C₅ alkyl or the acid is replaced with a tetrazole.

In one embodiment, the sortilin binding moiety (S_{L}) in the bifunctional compound according to formula (I) is a substituent derived from the compound 4-[(3,4-dichlorophenyl)amino]-3-(3-methylbenzyl)-4-oxobutanoic acid or 3-benzyl-4-[(3-chloro-2-methylphenyl)amino]-4-oxobutanoic acid.

In another embodiment, the sortilin binding moiety (S_{L}) in the bifunctional compound according to formula (I) is a substituent derived from a compound of formula XI:

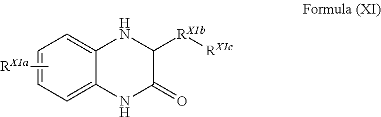

wherein
R^{XIa} is C₁₋₅ alkyl, acyl, amino, sulfono, chloro, bromo, iodo, or flouro;
R^{XIb} is a bond or a C₁-C₃ alkyl optionally substituted with a C₁-C₅ alkyl and
R^{XIc} is a carboxylic acid, a C₁-C₅ ester of a carboxylic acid or a tetrazole.

In one embodiment, the sortilin binding moiety (S_{L}) in the bifunctional compound according to formula (I) is a substituent derived from the compound 3-oxo-1,2,3,4-tetrahydro-2-quinoxalinyl) acetic acid.

Other sortilin binders are described in WO 2023/031440, which is hereby incorporated by reference, such as described by formula (A)

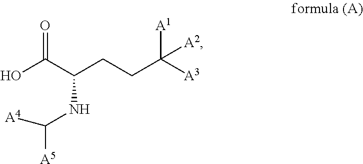

wherein
A¹, A² and A³ are each independently selected from the group consisting of halogen, H, C₁-C₄ alkyl, C₁-C₄ haloalkyl, C₂-C₅ alkenyl and C₂-C₅ haloalkenyl;
A⁴ is selected from the group consisting of H, C₁-C₃alkyl, C₁-C₃ haloalkyl, C₃-C₈ aryl, C₃-C₈ aryl with one or more halogen substituents, C₃-C₈ heteroaryl and C₃-C₈ heteroaryl with one or more halogen substituents;
A₅ is selected from the group consisting of C₃-C₂₀ aryl, C₃-C₂₀ heteroaryl and 3- to 12-membered-heterocyclic ring; wherein the aryl, heteroaryl or heterocyclic ring is optionally substituted with one or more substituents independently selected from halogen, —OH, cyano, carbonyl, C₁-C₄ alkyl, C₁-C₄ hydroxyalkyl, C₁-C₄ haloalkyl, acetyl, C₁-C₄ alkoxy, C₁-C₄ haloalkoxy, C₃-C₈ aryl and C₃-C₈ heteroaryl; or
A⁴ and A⁵ are taken together to form a 6- to 20-membered heterocyclic ring, wherein wherein the heterocyclic ring is monocyclic, bicyclic or tricyclic and is optionally substituted with one or more substituents independently selected from halo, —OH, cyano, carbonyl, C₁-C₄ alkyl, C₁-C₄ haloalkyl, acetyl, C₁-C₄ haloalkoxy, C₁-C₄ haloalkoxy.

For example, as the compounds below:

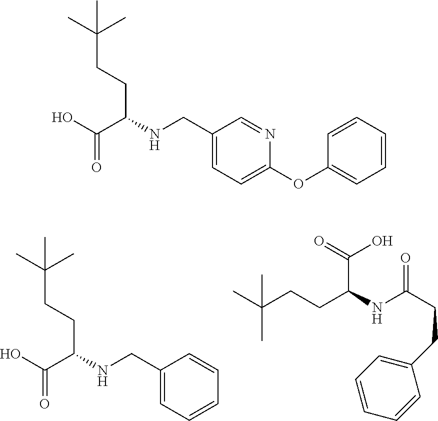

Thus, in one embodiment, the bifunctional compounds according to the present are able to bind sortilin through substituents derived from the above structures or derivatives thereof.

In one embodiment, the sortilin binding moiety (S_{L}) of the bifunctional compound is a substituent derived from a compound according to formula (A) above.

In one embodiment, the sortilin binding moiety (S_{L}) in the bifunctional compound according to formula (I) is according to any one of formulas A-I to A-III:

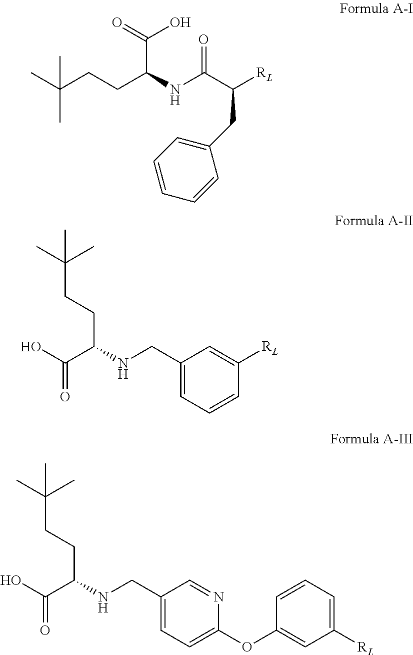

or a derivative thereof, wherein R^{L} denotes the attachment to L_{I}.

In one embodiment, the sortilin binding moiety (S_{L}) in the bifunctional compound is according to formula A-I. In one embodiment, the sortilin binding moiety (S_{L}) in the bifunctional compound is according to formula A-II. In one embodiment, the sortilin binding moiety (S_{L}) in the bifunctional compound is according to formula A-III.

Other sortilin binders are described in WO 2023/101595, the contents of which are hereby incorporated by reference, such as described by formula (E):

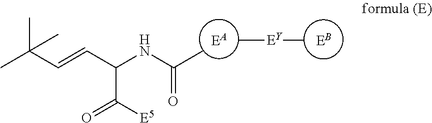

wherein
ring E^{A} is a 5 or 6 membered aromatic, heteroaromatic or heterocyclic ring with 0 to 2 heteroatom(s) selected among N, O and S, or a 8 to 10 membered bicyclic heterocyclic or heteroaromatic ring with 1 or 2 heteroatom(s) selected among N, O and S, the aromatic, heteroaromatic, heterocyclic or bicyclic ring A is optionally substituted with one or more substituents independently selected from the group consisting of C₁-C₄ alkyl, —N(E¹)(E²), —C(═O)C(E^{Z})₃, —OE⁴, halogen and ═O;
E^{Y} is absent, —O—, —OCH₂—, —CH₂—, —NE³-, or —CH(NH₂)—;
ring E^{B} is absent or a 5 or 6 membered aromatic or heteroaromatic ring with 0 to 4 heteroatom(s) selected among N, O and S, the aromatic or heteroaromatic ring E^{B} is optionally substituted with one or more substituents independently selected from the group consisting of C₁-C₄ alkyl, —N(E¹)(E²), —C(═O)C(E^{Z})₃, —OE⁴, halogen and ═O;
E¹, E², E³ and E⁴ are each independently selected from hydrogen or C₁-C₄ alkyl;
E^{Z} is halogen; and
E⁵ is hydroxyl or C₁-C₄ alkoxy.

In one embodiment, the sortilin binding moiety (S_{L}) of the bifunctional compound is a substituent derived from a compound according to formula (E) above.

In one aspect, the present invention provides novel sortilin ligands with high affinity to sortilin. Thus, one embodiment of the present disclosure provides compounds according to formula III, or pharmaceutically acceptable salts thereof:

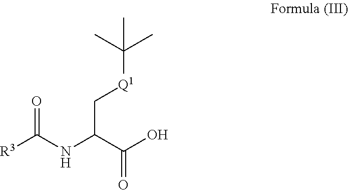

wherein Q¹ is a bond or —CH₂—; and
R³ is of Formula (IIIp) or Formula (IIIq) or formula B-II:

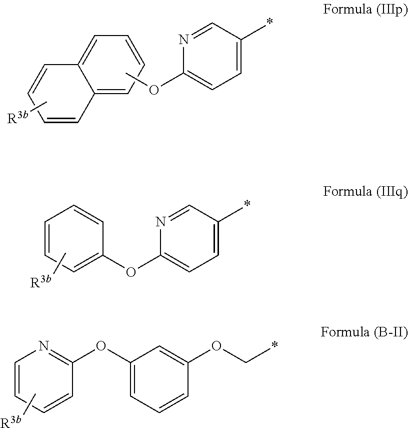

wherein R^{3b} is selected from H, halogen, alkoxy, —CF₃, and an optionally substituted C₁₋₅ alkyl, wherein one or more methylene group(s) of the C₁-C₅ alkyl are optionally individually replaced by one or more of the groups consisting of —O—, —NH—, —C(O)—, ester, amide, carbamate, thiourea, sulphonamide, urea,

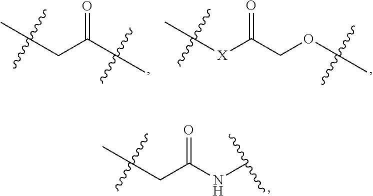

an optionally substituted carbocycle; an optionally substituted heterocycle and

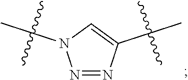

wherein X is NH or O; and
wherein * denotes the attachment with Formula (III).

In one embodiment, R^{3b} is not H when R³ is of formula (IIIq).

In one embodiment, Q¹ is —CH2-. In one embodiment, Q¹ is a bond.

In one embodiment, R³ is of formula IIIr:

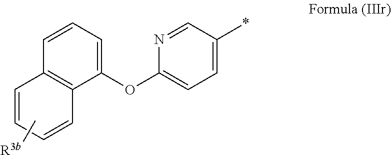

wherein R^{3b} is selected from H, halogen, alkoxy, —CF₃, and an optionally substituted C₁₋₅ alkyl, wherein one or more methylene group(s) of the C₁₋₅ alkyl are optionally individually replaced by one or more of the groups consisting of —O—, —NH—, —C(O)—, ester, amide, carbamate, thiourea or

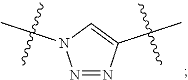 [SMILES: *c1cn(*)nn1]

and
wherein * denotes the attachment with Formula (III).

In one embodiment of the present disclosure the compound is selected from formula IIIs, formula IIIt or formula B-III:

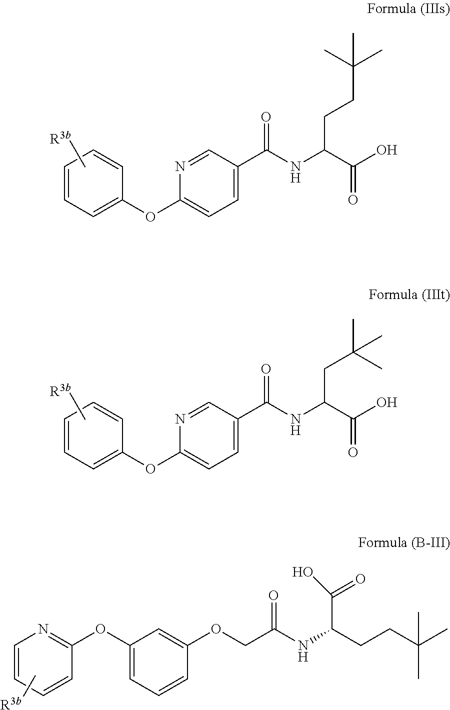

wherein R^{3b} is selected from H, halogen, alkoxy, —CF₃, and an optionally substituted C₁₋₅ alkyl, wherein one or more methylene group(s) of the C₁₋₅ alkyl are optionally individually replaced by one or more of the groups consisting of —O—, —NH—, —C(O)—, ester, amide, carbamate, thiourea, sulphonamide, urea,

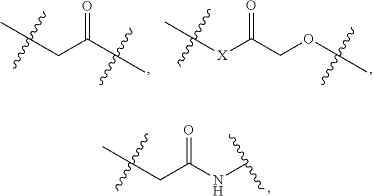

an optionally substituted carbocycle; an optionally substituted heterocycle and

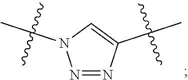

wherein X is NH or O.

In one embodiment, R^{3b} is H.

In one embodiment, R^{3b} is selected from halogen or —CF₃.

In one embodiment, R^{3b} is an optionally substituted C₁-C₅ alkyl, wherein one or more methylene group(s) of the C₁₋₅ alkyl are optionally individually replaced by one or more of the groups consisting of —O—, —NH—, —C(O)—, ester, amide, carbamate, thiourea and

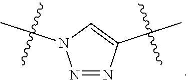 [SMILES: *c1cn(*)nn1]

In one embodiment, at least one of the methylene groups is replaced by one or more of the groups consisting of —O—, —NH—, —C(O)—, ester, amide, carbamate, thiourea and

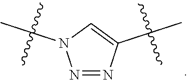 [SMILES: *c1cn(*)nn1]

In one embodiment, R^{3b} is C₁-C₅ alkyl, wherein one or more methylene group(s) of the C₁₋₅ alkyl are optionally individually replaced by one or more of the groups consisting of —O—, amide, and

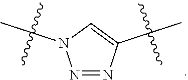 [SMILES: *c1cn(*)nn1]

In one embodiment, R^{3b} is C₁-C₅ alkyl, wherein one or more methylene group(s) of the C₁₋₅ alkyl are optionally individually replaced by —O—.

In one embodiment, R^{3b} is C₁-C₅ alkyl, wherein one or more methylene group of the C₁₋₅ alkyl is optionally individually replaced by an amide.

In one embodiment, R^{3b} is C₁-C₅ alkyl, wherein one or more methylene group of the C₁₋₅ alkyl is optionally individually replaced by

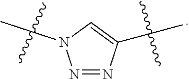 [SMILES: *c1cn(*)nn1]

In one embodiment, R^{3b} is C₁-C₅ alkyl, wherein one or more methylene group of the C₁₋₅ alkyl is optionally individually replaced by an optionally substituted carbocycle, such as a carbocycle according to

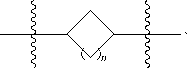

wherein n is an integer selected from 0, 1, 2 or 3.

In one embodiment, R^{3b} is C₁-C₅ alkyl, wherein one or more methylene group of the C₁₋₅ alkyl is optionally individually replaced by an optionally substituted heterocycle, such as an heterocycle according to

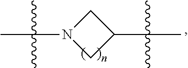

wherein n is an integer selected from 0, 1, 2 or 3; or an heterocycle according to

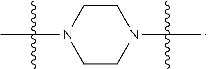 [SMILES: *N1CCN(*)CC1]

In one embodiment, R^{3b} is —O—CH₂—C(═O)N(H)R^{IIIa}, wherein R^{IIIa} is selected from H, and C₁-C₆ alkyl.

In one embodiment, R^{3b} is —O—CH₂—CO₂R^{IIIa}, wherein R^{IIIa} is selected from H, and C₁-C₆ alkyl.

In one embodiment, R^{3b} is C₁-C₅ alkyl, wherein one or more methylene group of the C₁₋₅ alkyl is optionally individually replaced by one of the groups shown in Table Z in the section “Linkers”. In one embodiment, the compound is the S-stereoisomer based on the configuration of the carbon in α-position from the carboxylic acid.

In one embodiment, the compound is the R-stereoisomer based on the configuration of the carbon in α-position from the carboxylic acid.

In one embodiment, the present disclosure provides the compounds:

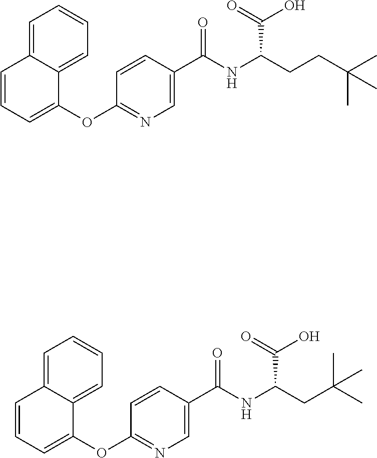 [SMILES: CC(C)(C)CC[C@H](NC(=O)c1ccc(Oc2cccc3ccccc23)nc1)C(=O)O ; CC(C)(C)C[C@H](NC(=O)c1ccc(Oc2cccc3ccccc23)nc1)C(=O)O]

In another embodiment, the present disclosure provides the following compounds:

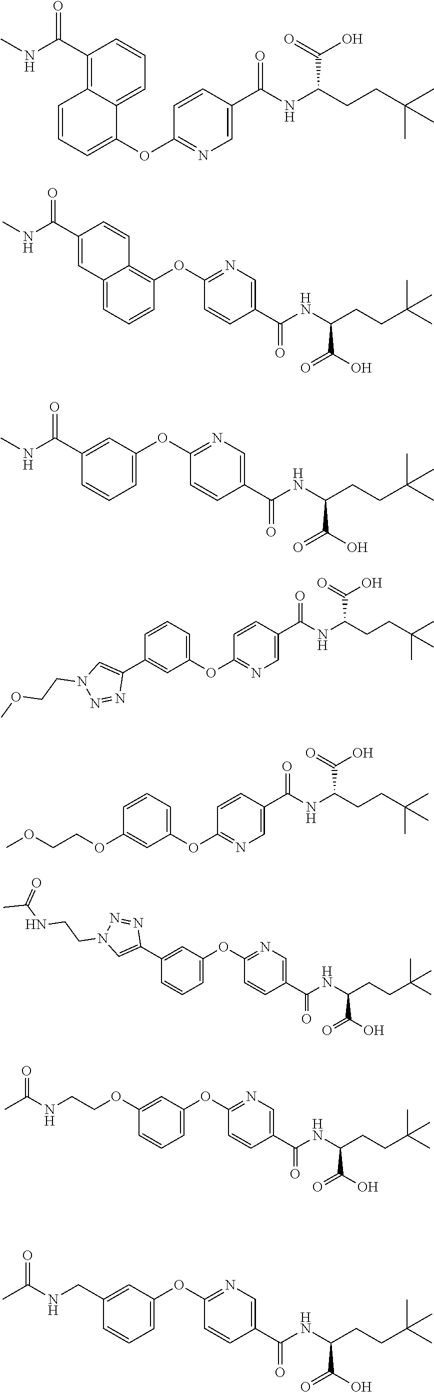

or a pharmaceutically acceptable salt thereof.

In one embodiment, the present disclosure provides the compounds:

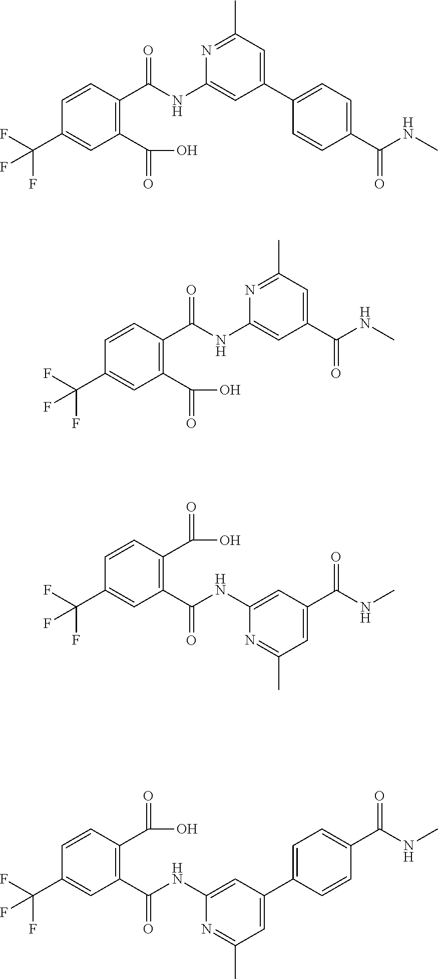 [SMILES: CNC(=O)c1ccc(-c2cc(C)nc(NC(=O)c3ccc(C(F)(F)F)cc3C(=O)O)c2)cc1 ; CNC(=O)c1cc(C)nc(NC(=O)c2ccc(C(F)(F)F)cc2C(=O)O)c1 ; CNC(=O)c1cc(C)nc(NC(=O)c2cc(C(F)(F)F)ccc2C(=O)O)c1 ; CNC(=O)c1ccc(-c2cc(C)nc(NC(=O)c3cc(C(F)(F)F)ccc3C(=O)O)c2)cc1]

or a pharmaceutically acceptable salt thereof.

In one aspect the present disclosure provides for a compound according to any one of:

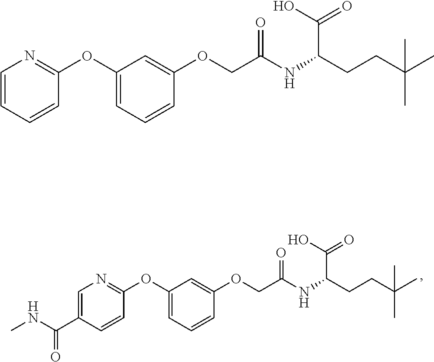

or a pharmaceutically acceptable salt thereof.

The present invention demonstrates several bifunctional compounds able to bind sortilin. As shown in the examples, these compounds have affinity for sortilin. As it will be understood to someone of skill in the art, fragments of the bifunctional compounds encompassing S_{L}, and a part or all of L_{I}, will also perform as sortilin binders. Thus, in one embodiment, the present disclosure provides for a sortilin binder as described by any combination of S_{L}-L_{I}, or a fragment thereof, as described herein below.

In one embodiment, the present invention provides for compounds according to any one of formulas IIIt or B-III, wherein R^{3b} is a fragment of any one of the structures shown in table Z-III of the section “Linkers”.

In addition, the present disclosure provides for a compound able to bind sortilin, wherein said compound is an intermediate in the preparation of a bifunctional compound as described herein in the section “Synthetic protocols”.

### Peptides

Peptides have also been shown to bind sortilin. For example, the neuropeptide neurotensin (SEQ ID NO.: 4) and fragments thereof, such as 4 amino acid C-terminal neurotensin fragment (SEQ ID NO.: 5) or the amidated 6 amino acid N-terminal neurotensin fragment (SEQ ID NO.: 6). Smaller peptide fragments are also able to bind sortilin, for instance, the C-terminal sequence of progranulin (SEQ ID NO.: 3), a natural sortilin binder, has been shown to be essential in the binding of progranulin to sortilin.

Other examples of synthetic peptides have been used for targeting sortilin, such as the peptides described in WO 2020/037434, for example:

(SEQ ID NO.: 7)
IKLSGGVQAKAGVINMDKSESM
(SEQ ID NO.: 8)
IKLSGGVQAKAGVINMFKSESY
(SEQ ID NO.: 9)
IKLSGGVQAKAGVINMFKSESYK
(SEQ ID NO.: 10)
GVQAKAGVINMFKSESY
(SEQ ID NO.: 11)
GVRAKAGVRNMFKSESY
(SEQ ID NO.: 12)
GVRAKAGVRN(Nle)FKSESY
(SEQ ID NO.: 13)
YKSLRRKAPRWDAPLRDPALRQLL
(SEQ ID NO.: 14)
YKSLRRKAPRWDAYLRDPALRQLL
(SEQ ID NO.: 15)
YKSLRRKAPRWDAYLRDPALRPLL

The sortilin binding group (S_{L}) in the bifunctional compounds according to the present invention may also be a peptide.

There exist several proteins with affinity to sortilin. Thus, in one embodiment of the present disclosure, the sortilin binding group (S_{L}) in the bifunctional compound comprises a peptide fragment of an amino acid sequence of such a protein.

In one embodiment, the sortilin binding group is comprises peptide fragment of an amino acid sequence of a protein selected form the group consisting of:
a) progranulin (SEQ ID NO.: 3),
b) neurotensin (SEQ ID NO.: 4),
c) brain derived neurotrophic factor (BDNF) (SEQ ID NO.: 177)
d) apolipoprotein B (ApoB) (SEQ ID NO.: 176)
e) nerve growth factor (NGF) (SEQ ID NO.: 178)
   or a variant of said fragment, having at least 60% sequence identity with the corresponding original protein fragment of any one of a) through e), such as at least 80%, such as at least 90%, such as at least 95% sequence identity.

In one embodiment, S_{L} comprises or consists of a peptide fragment of an amino acid sequence of progranulin (SEQ ID NO.: 3), or a variant of said fragment having at least 60% sequence identity with the corresponding original fragment of SEQ ID NO.: 3, such as at least 80% sequence identity, such as at least 90%, such as at least 95%, such as at least 99% sequence identity.

In one embodiment, S_{L} comprises or consists of a peptide fragment of an amino acid sequence of progranulin (SEQ ID NO.: 3), or a variant of said fragment having up to 5 amino acids substitutions compared to the corresponding original fragment of SEQ ID NO.: 3, such up to 4 amino acid substitutions, such as up to 3 amino acid substitutions, such as up to 2 amino acids substitution, such as up to 1 amino acid substitution.

In one embodiment, S_{L} comprises or consists of a peptide fragment of an amino acid sequence of neurotensin (SEQ ID NO.: 4), or a variant of said fragment having at least 60% sequence identity with the corresponding original fragment of SEQ ID NO.: 4, such as at least 80% sequence identity, such as at least 90%, such as at least 95%, such as at least 99% sequence identity.

In one embodiment, S_{L} comprises or consists of a peptide fragment of an amino acid sequence of neurotensin (SEQ ID NO.: 4), or a variant of said fragment having up to 5 amino acids substitutions compared to the corresponding original fragment of SEQ ID NO.: 4, such up to 4 amino acid substitutions, such as up to 3 amino acid substitutions, such as up to 2 amino acids substitution, such as up to 1 amino acid substitution.

In one embodiment, S_{L} comprises or consists of a peptide fragment of an amino acid sequence of ApoB (SEQ ID NO.: 176), or a variant of said fragment having at least 60% sequence identity with the corresponding original fragment of SEQ ID NO.: 176, such as at least 80% sequence identity, such as at least 90%, such as at least 95%, such as at least 99% sequence identity.

In one embodiment, S_{L} comprises or consists of a peptide fragment of an amino acid sequence of ApoB (SEQ ID NO.: 176), or a variant of said fragment having up to 5 amino acids substitutions compared to the corresponding original fragment of SEQ ID NO.: 176, such up to 4 amino acid substitutions, such as up to 3 amino acid substitutions, such as up to 2 amino acids substitution, such as up to 1 amino acid substitution.

In one embodiment, S_{L} comprises or consists of a peptide fragment of an amino acid sequence of BDNF (SEQ ID NO.: 177), or a variant of said fragment having at least 60% sequence identity with the corresponding original fragment of SEQ ID NO.: 177, such as at least 80% sequence identity, such as at least 90%, such as at least 95%, such as at least 99% sequence identity.

In one embodiment, S_{L} comprises or consists of a peptide fragment of an amino acid sequence of BDNF (SEQ ID NO.: 177), or a variant of said fragment having up to 5 amino acids substitutions compared to the corresponding original fragment of SEQ ID NO.: 177, such up to 4 amino acid substitutions, such as up to 3 amino acid substitutions, such as up to 2 amino acids substitution, such as up to 1 amino acid substitution.

In one embodiment, S_{L} comprises or consists of a peptide fragment of an amino acid sequence of NGF (SEQ ID NO.: 178), or a variant of said fragment having at least 60% sequence identity with the corresponding original fragment of SEQ ID NO.: 178, such as at least 80% sequence identity, such as at least 90%, such as at least 95%, such as at least 99% sequence identity.

In one embodiment, S_{L} comprises or consists of a peptide fragment of an amino acid sequence of NGF (SEQ ID NO.: 178), or a variant of said fragment having up to 5 amino acids substitutions compared to the corresponding original fragment of SEQ ID NO.: 178, such up to 4 amino acid substitutions, such as up to 3 amino acid substitutions, such as up to 2 amino acids substitution, such as up to 1 amino acid substitution.

In one embodiment, S_{L} is a peptide comprising a sequence from the group consisting of:

(SEQ ID NO.: 44)
H-PYMKLAPGELTIIL-OH,
(SEQ ID NO.: 45)
H-NEKLSQLQTYMI-OH,
(SEQ ID NO.: 46)
H-KDADLYTSRVMLSSQVP-OH
(SEQ ID NO.: 47)
H-RLFKKRRLRSPRVLF-NH2,
(SEQ ID NO.: 48)
H-ITVDPRLFKKRRLRSPRVLF-NH2,
(SEQ ID NO.: 49)
H-ITVDPRLFKKRRLRSPRVLFSTQPPR-OH,
(SEQ ID NO.: 50)
H-WSGPIGVSWGLRAAAAGGAFP-OH,
(SEQ ID NO.: 51)
H-WSGPIGVSWGLRAAAAGGAFPRGGRWRR-OH,
(SEQ ID NO.: 52)
H-GVSWGLR-OH
(SEQ ID NO.: 179)
WSGPIGVSWGLRAAAAGFQLL-OH.

When S_{L} comprises of consists of a peptide, L_{I} may be connected at any amino acid residue of the peptide. In one embodiment, when S_{L} comprises a peptide, L_{I} is connected at any free amino group of the peptide.

In a preferred embodiment, when S_{L} comprises or consists of a peptide, L_{I} is connected to the N-terminus of the peptide.

The inventors have shown that the C-terminal tetrapeptide of progranulin RQLL (SEQ ID NO.:) is sufficient to bind sortilin and promote lysosomal mediated degradation of progranulin. Furthermore, the inventors have shown that several peptide analogs with selected substitutions on the RQLL sequence are good binders of sortilin.

Thus in one embodiment, the S_{L} comprises or consists of a peptide comprising the following sequence SEQ ID NO.: 16

(SEQ ID NO.: 16)
X₅-X₁X₂X₃X₄

wherein,
X₅ is an optional amino acid residue or peptide comprising 2 to 30 amino acid residues or a bond,
X₁ is R, P, F, Y, L, K, G or H;
X₂ is Q, Y, L, E or G;
X₃ is Y, F, L, I, Q, E or N; and
X₄ is M, K, L or a conservative substitution of L.

In one embodiment, X₅ is an optional amino acid residue or peptide comprising at least 2 amino acid residues, such as at least 3, such as at least 4, such as at least 5, such as at least 6, such as at least 7, such as at least 8, such as at least 9, such as at least 10, such as at least 11, such as at least 12, such as at least 13, such as at least 14, such as at least 15, such as at least 20, such as at least 25 amino acid residues, such as at least 28 amino acid residues, such as at least 30 amino acid residues.

In one embodiment, X₅ is a peptide comprising no more than 32 amino acid residues, such as no more than 29, such as no more than 28, such as no more than 27, such as no more than 26, such as no more than 25, such as no more than 24, such as no more than 23, such as no more than 22, such as no more than 21, such as no more than 20, such as no more than 15, such as no more than 10, such as no more than 5 amino acid residues.

In one embodiment, X₅ is a peptide fragment of an amino acid sequence of a protein selected from the group consisting of:
a) progranulin (SEQ ID NO.: 3),
b) neurotensin (SEQ ID NO.: 4),
c) brain derived neurotrophic factor (BDNF) (SEQ ID NO.: 177)
d) apolipoprotein B (ApoB) (SEQ ID NO.: 176)
e) nerve growth factor (SEQ ID NO.: 178)
   or a variant of said fragment, having at least 60% sequence identity with the corresponding original protein fragment of any one of a) through e), such as at least 80%, such as at least 90%, such as at least 95% sequence identity.

In one embodiment, X₅ comprises or consists of a peptide fragment of an amino acid sequence of progranulin (SEQ ID NO.: 3), or a variant of said fragment having at least 60% sequence identity with the corresponding original fragment of SEQ ID NO.: 3, such as at least 80% sequence identity, such as at least 90%, such as at least 95%, such as at least 99% sequence identity.

In one embodiment, X₅ comprises or consists of a peptide fragment of an amino acid sequence of progranulin (SEQ ID NO.: 3), or a variant of said fragment having up to 5 amino acids substitutions compared to the corresponding original fragment of SEQ ID NO.: 3, such up to 4 amino acid substitutions, such as up to 3 amino acid substitutions, such as up to 2 amino acids substitution, such as up to 1 amino acid substitution.

In one embodiment, X₅ comprises or consists of a peptide fragment of an amino acid sequence of neurotensin (SEQ ID NO.: 4), or a variant of said fragment having at least 60% sequence identity with the corresponding original fragment of SEQ ID NO.: 4, such as at least 80% sequence identity, such as at least 90%, such as at least 95%, such as at least 99% sequence identity.

In one embodiment, X₅ comprises or consists of a peptide fragment of an amino acid sequence of neurotensin (SEQ ID NO.: 4), or a variant of said fragment having up to 5 amino acids substitutions compared to the corresponding original fragment of SEQ ID NO.: 4, such up to 4 amino acid substitutions, such as up to 3 amino acid substitutions, such as up to 2 amino acids substitution, such as up to 1 amino acid substitution.

In one embodiment, X₅ is selected from the group consisting of:
an amino acid residue selected from L, T or Y;

(SEQ ID NO.: 17)
REAPRWDAPLRDPAL;
(SEQ ID NO.: 18)
REALRWDAPLRDPAP;
(SEQ ID NO.: 19)
PYILKRQLYENKPRR;
(SEQ ID NO.: 20)
LYENKPR;
and
(SEQ ID NO.: 21)
APLRDPAP
(SEQ ID NO.: 180)
WSGPIGVSWGLRAAAAG
(SEQ ID NO.: 181)
CREAPRWDAPLRDPAL

or a variant thereof having 1 to 5 amino acids substitutions.

In one embodiment, X₅ is a bond.

In one embodiment, X₅ is APLRDPAP (SEQ ID NO.: 21). In one embodiment, X₅ is REAPRWDAPLRDPAL (SEQ ID NO.: 17). In one embodiment, X₅ is

(SEQ ID NO.: 18)
REALRWDAPLRDPAP.

In one embodiment, X⁵ is an amino acid residue selected from L, T or Y. In one embodiment, X⁵ PYILKRQLYENKPRR (SEQ ID NO.: 19). In one embodiment, X⁵ LYENKPR (SEQ ID NO.: 20).

In one embodiment, X¹ is F or R. In one embodiment, X² is Q. In another embodiment, X³ is L, in one embodiment X₄ is L.

In one embodiment,
X₁ is selected from Y, F, R, P, L or H,
X₂ is selected from Q, Y or L,
X₃ is selected from Y, F, L, I or Q,
X₄ is L.

In one embodiment,
X₁ is selected from Y, F, R or P,
X₂ is selected from Q or Y,
X₃ is selected from L, Y, F or I,
X₄ is L.

In one embodiment,
X₁ is selected from Y, F or R,
X₂ is selected from Q or Y,
X₃ is L,
X₄ is L.

In one embodiment,
X₁ is selected from F or R,
X₂ is Q,
X₃ is L,
X₄ is L.

In one embodiment, S_{L} comprises or consists of a sequence selected from any one of the group consisting of SEQ ID NOs: 22 to 65, 182 to 187, 196 and 197, wherein L_{I} is connected at the N-terminus.

In one embodiment, S_{L} is a peptide comprising any of the sortilin binding sequences described herein at the C-terminus.

In one embodiment, S_{L} is a peptide consisting of RQLL-OH (SEQ ID NO.: 22). In one embodiment S_{L}, is a peptide comprising RQLL-OH (SEQ ID NO.: 22) at the C-terminus.

In one embodiment, S_{L} is a peptide consisting of FQLL-OH (SEQ ID NO.: 23). In one embodiment S_{L}, is a peptide comprising FQLL-OH (SEQ ID NO.: 23) at the C-terminus.

In one embodiment, S_{L} is a peptide consisting of RYLL-OH (SEQ ID NO.: 27). In one embodiment S_{L}, is a peptide comprising RYLL-OH (SEQ ID NO.: 27) at the C-terminus.

In one embodiment, S_{L} is a peptide consisting of FYLL-OH (SEQ ID NO.: 28). In one embodiment S_{L}, is a peptide comprising FYYL-OH (SEQ ID NO.: 28) at the C-terminus.

In one embodiment, S_{L} is a peptide consisting of YQLL-OH (SEQ ID NO.: 29). In one embodiment S_{L}, is a peptide comprising YQLL-OH (SEQ ID NO.: 29) at the C-terminus.

In one embodiment, S_{L} is a peptide consisting of REAPRWDAPLRDPALRQLL-OH (SEQ ID NO.: 53). In one embodiment S_{L}, is a peptide comprising REAPRWDAPLRDPALRQLL-OH (SEQ ID NO.: 53) at the C-terminus.

In one embodiment, S_{L} is a peptide consisting of REALRWDAPLRDPAPRQLL-OH (SEQ ID NO.: 54). In one embodiment S_{L}, is a peptide comprising REALRWDAPLRDPAPRQLL-OH (SEQ ID NO.: 54) at the C-terminus.

In one embodiment, S_{L} is a peptide consisting of REAPRWDAPLRDPALFQLL-OH (SEQ ID NO.: 58). In one embodiment S_{L}, is a peptide comprising REAPRWDAPLRDPALFQLL-OH (SEQ ID NO.: 58) at the C-terminus.

In one embodiment, S_{L} is a peptide consisting of REAPRWDAPLRDPALRYLL-OH (SEQ ID NO.: 59). In one embodiment S_{L}, is a peptide comprising REAPRWDAPLRDPALRYLL-OH (SEQ ID NO.: 59) at the C-terminus.

In one embodiment, S_{L} is a peptide consisting of REAPRWDAPLRDPALRQYL-OH (SEQ ID NO.: 60). In embodiment S_{L}, is one a peptide comprising REAPRWDAPLRDPALRQYL-OH (SEQ ID NO.: 60) at the C-terminus.

In one embodiment, S_{L} is a peptide consisting of REAPRWDAPLRDPALRYYL-OH (SEQ ID NO.: 61). In one embodiment S_{L}, s a peptide comprising REAPRWDAPLRDPALRYYL-OH (SEQ ID NO.: 61) at the C-terminus.

In one embodiment, S_{L} is a peptide consisting of APLRDPAPRQLL-OH (SEQ ID NO.: 57). In one embodiment S_{L}, is a peptide comprising APLRDPAPRQLL-OH (SEQ ID NO.: 57) at the C-terminus.

In one embodiment, S_{L} is a peptide consisting of PYILKRQLYENKPRRPYIL-OH (SEQ ID NO.: 55). In one embodiment S_{L}, is a peptide comprising PYILKRQLYENKPRRPYIL-OH (SEQ ID NO.: 55) at the C-terminus.

In one embodiment, S_{L} is a peptide consisting of LYENKPRRPYIL-OH (SEQ ID NO.: 56). In one embodiment S_{L}, is a peptide comprising LYENKPRRPYIL-OH (SEQ ID NO.: 56) at the C-terminus.

In one embodiment, S_{L} is a peptide consisting of AARL-OH (SEQ ID NO.: 196). In one embodiment S_{L}, is a peptide comprising AARL-OH (SEQ ID NO.: 196) at the C-terminus.

In one embodiment, S_{L} is a peptide consisting of PIPLV-OH (SEQ ID NO.: 197). In one embodiment S_{L}, is a peptide comprising PIPLV-OH (SEQ ID NO.: 197) at the C-terminus.

In one preferred embodiment, L_{I} is connected to S_{L} at the N-terminus.

In one embodiment, 1 to 4 amino acids residues of S_{L} are substituted with other amino acids, such as one amino acid is substituted, such as 2, such as 3, such as 4 amino acids.

In one embodiment, 1 amino acid residue of St is substituted. In another embodiment, 2 amino acid residues of S_{L} are substituted.

In one embodiment, the substitution is a conservative amino acid substitution. In another embodiment, the substitution is with a non-naturally occurring amino acid. Thus, in one embodiment, S_{L} may have 1 substitution with 1 non-naturally occurring amino acid. In another embodiment, S_{L} may have two substitutions with a non-naturally occurring amino acids.

In one embodiment, 1 to 4 amino acid residues of S_{L} are chemically modified, such as 1 amino acid residue is modified, such as 2, such as 3, such as 4 amino acids.

In one embodiment, the chemical modification may be any chemical modification. In one embodiment, the chemical modification is selected from the group consisting of acylation, amidation, acetylation, esterification and/or alkylation. Thus, S_{L} may have 1 chemically modified amino acids. In another embodiment, S_{L} may have 2 chemically modified amino acids.

In one embodiment, S_{L} is a peptide that comprises no more than 50 amino acid residues, such as no more than 45, such as no more than 40, such as no more than 35, such as no more than 32, such as no more than 30, such as no more than 28, such as no more than 26, such as no more than 24, such as no more than 22, such as no more than 20, such as no more than 19, such as no more than 18, such as no more than 17, such as no more than 16, such as no more than 15, such as no more than 14, such as no more than 13, such as no more than 12, such as no more than 11, such as no more than 10, such as no more than 9, such as no more than 8, such as no more than 7, such as no more than 6, such as no more than 5, such as no more than 4 amino acid residues.

In one embodiment, S_{L} is a peptide that comprises no more than 28 amino acid residues. In another embodiment, S_{L} is a peptide that comprises no more than 4 amino acid residues.

In one embodiment, S_{L} comprises or consists of a peptide that is at least 4 amino acid residues, such as at least 5, such as at least 6, such as at least 7, such as at least 8, such as at least 9, such as at least 10, such as at least 12, such as at least 14, such as at least 16, such as at least 18, such as at least 20, such as at least 22, such as at least 24, such as at least 26, such as at least 28.

In one embodiment, S_{L} comprises or consists of a peptide with a length of 4 to 50 amino acids, such as 4 to 32 amino acids, for example 4 to 28 amino acids, such as 4 to 24 amino acids, such as 4 to 20 amino acids, such as 4 to 19 amino acids, such as 4 to 18, such as 4 to 17 amino acids, such as 4 to 16 amino acids, such as 4 to 15 amino acids, such as 4 to 14 amino acids, such as 4 to 13 amino acids, such as 4 to 12 amino acids, such as 4 to 11 amino acids, such as 4 to 10 amino acids, such as 4 to 9 amino acids, such as 4 to 8 amino acids, such as 4 to 7 amino acids, such as 4 to 6 amino acids, such as 4 to 5 amino acids.

In one embodiment, S_{L} comprises or consists of a peptide with a length of 4 amino acid residues. In one embodiment, S_{L} comprises or consists of a peptide with a length of 5 amino acid residues. In one embodiment, S_{L} comprises or consists of a peptide with a length of 6 amino acid residues. In one embodiment, S_{L} comprises or consists of a peptide with a length of 7 amino acid residues. In one embodiment, S_{L} comprises or consists of a peptide with a length of 8 amino acid residues. In one embodiment, S_{L} comprises or consists of a peptide with a length of 9 amino acid residues. In one embodiment, S_{L} comprises or consists of a peptide with a length of 10 amino acid residues. In one embodiment, S_{L} comprises or consists of a peptide with a length of 11 amino acid residues. In one embodiment, S_{L} comprises or consists of a peptide with a length of 12 amino acid residues.

In one embodiment, S_{L} comprises or consists of a peptide able to bind sortilin with a dissociation constant of less than 50 μM, such as less than 40 μM, such as less than 30 μM, such as less than 20 μM, such as less than 10 μM, such as less than 5 μM, such as less than 4 μM, such as less than 3 μM, such as less than 2 μM, such as less than 1 μM, such as less than 0.8 μM, such as less than 0.6 μM, such as less than 0.5 μM, such as less than 0.4 μM, such as less than 0.3 μM, such as less than 0.2 μM, such as less than 0.1 μM, such as less than 0.05 μM, such as less than 0.04 μM, such as less than 0.03 μM, such as less than 0.02 μM such as less than 0.01 μM.

It is one aspect of the present invention, to provide sortilin binding peptides. In one embodiment, such a peptide is according to St as disclosed herein.

It is one aspect of the present invention, to provide sortilin binding peptides. In one embodiment, the sortilin binding peptide comprises the following sequence:

(SEQ ID NO.: 16)
X₅-X₁X₂X₃X₄

wherein X₁, X₂, X₃, X₄ and X₅ are defined as described herein.

In one embodiment, the peptide is a peptide with a length of 4 to 50 amino acids, such as 4 to 32 amino acids, for example 4 to 28 amino acids, such as 4 to 24 amino acids, such as 4 to 20 amino acids, such as 4 to 18 amino acids, such as 4 to 14 amino acids, such as 4 to 12 amino acids, such as 4 to 11 amino acids, such as 4 to 10 amino acids, such as 4 to 9 amino acids, such as 4 to 8 amino acids, such as 4 to 7 amino acids, such as 4 to 6 amino acids, such as 4 to 5 amino acids.

In one embodiment, the peptide is a peptide consisting of RQLL-OH (SEQ ID NO.: 22). In one embodiment the peptide is a peptide comprising RQLL-OH (SEQ ID NO.: 22) at the C-terminus.

In one embodiment, the peptide is a peptide consisting of FQLL-OH (SEQ ID NO.: 23). In one embodiment the peptide is a peptide comprising FQLL-OH (SEQ ID NO.: 23) at the C-terminus.

In one embodiment, the peptide is a peptide consisting of RYLL-OH (SEQ ID NO.: 27). In one embodiment the peptide, is a peptide comprising RYLL-OH (SEQ ID NO.: 27) at the C-terminus.

In one embodiment, the peptide is a peptide consisting of FYLL-OH (SEQ ID NO.: 28). In one embodiment the peptide is a peptide comprising FYYL-OH (SEQ ID NO.: 28) at the C-terminus.

In one embodiment, the peptide is a peptide consisting of YQLL-OH (SEQ ID NO.: 29). In one embodiment the peptide is a peptide comprising YQLL-OH (SEQ ID NO.: 29) at the C-terminus.

In one embodiment, the peptide is a peptide consisting of REAPRWDAPLRDPALRQLL-OH (SEQ ID NO.: 53). In one embodiment the peptide is a peptide comprising REAPRWDAPLRDPALRQLL-OH (SEQ ID NO.: 53) at the C-terminus.

In one embodiment, the peptide is a peptide consisting of REALRWDAPLRDPAPRQLL-OH (SEQ ID NO.: 54). In one embodiment the peptide is a peptide comprising REALRWDAPLRDPAPRQLL-OH (SEQ ID NO.: 54) at the C-terminus.

In one embodiment, the peptide is a peptide consisting of REAPRWDAPLRDPALFQLL-OH (SEQ ID NO.: 58). In one embodiment the peptide, is a peptide comprising REAPRWDAPLRDPALFQLL-OH (SEQ ID NO.: 58) at the C-terminus.

In one embodiment, the peptide is a peptide consisting of REAPRWDAPLRDPALRYLL-OH (SEQ ID NO.: 59). In one embodiment the peptide, is a peptide comprising REAPRWDAPLRDPALRYLL-OH (SEQ ID NO.: 59) at the C-terminus.

In one embodiment, the peptide is a peptide consisting of REAPRWDAPLRDPALRQYL-OH (SEQ ID NO.: 60). In one embodiment the peptide is a peptide comprising REAPRWDAPLRDPALRQYL-OH (SEQ ID NO.: 60) at the C-terminus.

In one embodiment, the peptide is a peptide consisting of REAPRWDAPLRDPALRYYL-OH (SEQ ID NO.: 61). In one embodiment the peptide is a peptide comprising REAPRWDAPLRDPALRYYL-OH (SEQ ID NO.: 61) at the C-terminus.

In one embodiment, the peptide is a peptide consisting of APLRDPAPRQLL-OH (SEQ ID NO.: 57). In one embodiment the peptide is a peptide comprising APLRDPAPRQLL-OH (SEQ ID NO.: 57) at the C-terminus.

In one embodiment, the peptide is a peptide consisting of PYILKRQLYENKPRRPYIL-OH (SEQ ID NO.: 55). In one embodiment the peptide is a peptide comprising PYILKRQLYENKPRRPYIL-OH (SEQ ID NO.: 55) at the C-terminus.

In one embodiment, the peptide is a peptide consisting of LYENKPRRPYIL-OH (SEQ ID NO.: 56). In one embodiment the peptide, is a peptide comprising LYENKPRRPYIL-OH (SEQ ID NO.: 56) at the C-terminus.

In one embodiment, the peptide according to the present disclosure is able to bind sortilin with a dissociation constant (K_{D}) of less than 50 μM, such as less than 40 μM, such as less than 30 μM, such as less than 20 μM, such as less than 10 μM, such as less than 5 μM, such as less than 4 μM, such as less than 3 μM, such as less than 2 μM, such as less than 1 μM, such as less than 0.8 μM, such as less than 0.6 μM, such as less than 0.5 μM, such as less than 0.4 μM, such as less than 0.3 μM, such as less than 0.2 μM, such as less than 0.1 μM, such as less than 0.05 μM, such as less than 0.04 μM, such as less than 0.03 μM, such as less than 0.02 μM such as less than 0.01 μM.

In one aspect, the present disclosure provides for an isolated polynucleotide encoding for the peptide or the protein as described herein.

In one aspect, the present disclosure provides for a vector comprising the polynucleotide as described herein. In one embodiment, the vector according is an expression vector, such as a bacterial vector or a viral vector.

In one aspect, the present disclosure provides for a host cell comprising the polynucleotide and/or the vector described herein.

### Proteins

Bifunctional compounds according to the invention may be prepared by using any antibody or antibody fragment able to bind sortilin. Hence, according to the present disclosure, S_{L} may be selected from an antibody or an antibody fragment able to bind sortilin.

Antibodies or antibody fragments with the ability to bind sortilin have been disclosed, such as described in WO 2017/009327, WO 2019/016247, WO 2021/263279, WO 2021/116290, WO 2020/252066, WO 2020/014617, US 2017/0218058 or WO 2016/164637, the contents of which are hereby incorporated by reference.

Thus, in one embodiment, S_{L} is selected from one of the antibody or antibody fragments able to bind sortilin described in the above-mentioned disclosures. In one embodiment, S_{L} is selected from an antibody or antibody fragment as described in the above-mentioned disclosures, and L_{I} and/or T_{L} are selected as described herein.

In one embodiment, S_{L} is an antibody, an antibody fragment or a nanobody able to bind sortilin.

In one embodiment, S_{L} is an antibody, an antibody fragment or a nanobody able to bind sortilin with a dissociation constant of at least 50 μM, such as at least 2 μM, such as at least 0.1 μM.

As seen above, examples of sortilin binders suitable for preparing bifunctional compounds according to the present disclosure are many. Thus, in one embodiment the S_{L} group in the bifunctional compound is selected from any one of the above mentioned structures, peptides and proteins.

In one embodiment, the bifunctional compound according to the present disclosure is able to bind sortilin with a dissociation constant (K_{D}) of less than 50 μM, such as less than 40 μM, such as less than 30 μM, such as less than 20 μM, such as less than 10 μM, such as less than 5 μM, such as less than 4 μM, such as less than 3 μM, such as less than 2 μM, such as less than 1 μM, such as less than 0.8 μM, such as less than 0.6 μM, such as less than 0.5 μM, such as less than 0.4 μM, such as less than 0.3 μM, such as less than 0.2 μM, such as less than 0.1 μM, such as less than 0.05 μM, such as less than 0.04 μM, such as less than 0.03 μM, such as less than 0.02 μM such as less than 0.01 μM.

In one embodiment, the bifunctional compound according to the present disclosure is able to bind sortilin with a dissociation constant (K_{D}) between 50 μM and 0.001 μM, such as between 50 μM and 40 μM, such as between 40 μM and 30 μM, such as between 30 μM and 20 μM, such as between 20 μM and 10 μM, such as between 10 μM and 5 μM, such as between 5 μM and 4 μM, such as between 4 μM and 3 μM, such as between 3 μM and 2 μM, such as between 2 μM and 1 μM, such as between 1 μM and 0.9 μM, such as between 0.9 μM and 0.8 μM, such as between 0.8 μM and 0.7 μM, such as between 0.7 μM and 0.6 μM, such as between 0.5 μM and 0.4 μM, such as between 0.4 μM and 0.3 μM, such as between 0.3 μM and 0.2 μM, such as between 0.2 μM and 0.1 μM, such as between 0.1 μM and 0.05 μM, such as between 0.05 μM and 0.01 μM, such as between 0.01 μM and 0.001 μM.

### Targeting Warhead:

The bifunctional compounds according to the present disclosure comprise a moiety able of binding an extracellular target molecule or protein, such as a growth factor, a cytokine, a hormone, a lipoprotein, a neurotransmitter, a capsid, an extracellular secreted protein, an antibody.

In one embodiment, the extracellular target molecule is a protein. An extracellular protein, as described herein refers to proteins that are not fully enclosed inside a cell. This means for example, a protein that is completely outside of a cell, but also membrane-bound or membrane associated proteins with an extracellular domain.

The bifunctional compounds according to formula (I) bind the extracellular target molecule through the target ligand (T_{L}). In an embodiment, T_{L} is a substituent of a small organic molecule (i.e., a non-biologic) that adequately binds to the target molecule, or a substituent derived from a pharmaceutically active compound that binds to the target extracellular protein, or a peptide, protein or biologic or a binding fragment thereof that adequately binds to the extracellular target molecule.

The T_{L} moiety may be for example, but not limited to, a substituent derived from of an approved or clinical stage drug, or a substituent derived from of a compound that would be reviewed as a drug by a regulatory organization such as the FDA or EMA.

The extracellular target protein can be any amino acid sequence to which the bifunctional compound comprising a T_{L} can be bound which through degradation thereof, results in a beneficial therapeutic effect. In one embodiment, the target protein is a non-endogenous peptide such as that from a pathogen or toxin. In another embodiment, the Target Protein can be an endogenous protein that mediates a disorder. The endogenous protein can be either the normal form of the protein or an aberrant form. For example, the Target Protein can be an extracellular mutant protein, or a protein, for example, where a partial, or full, gain-of-function or loss-of-function is encoded by nucleotide polymorphisms. In some embodiments, the bifunctional compound targets the aberrant form of the protein and not the normal form of the protein.

The Targeting Ligand (T_{L}) moiety of the bifunctional compound according to this disclosure is a ligand which covalently or non-covalently binds to a Target Protein which has been selected for lysosomal degradation. A Targeting Ligand is a small molecule or moiety (for example a peptide, nucleotide, antibody fragment, aptamer, biomolecule, or other chemical structure) that binds to a Target Protein, and wherein the Target Protein is a mediator of disease in a host as described in detail below.

A number of exemplary extracellular proteins targeted for medical therapy described below have characterizing structural information in the well-known Protein Data Bank (“PDB”), which is a database for the three-dimensional structural information for large biological molecules such as proteins and nucleic acids. PDB includes x-ray crystallography and other information submitted by scientists around the world and is freely accessible. See for example www.rcsb.org: wwwrwwpdb.org and www.uniprot.org in connection with the codes provided below.

For example, the skilled artisan can use available visualization tools, including those available on the PDB website, to determine where T_{L} docks into to the Extracellular Protein. The skilled artisan can also import the crystal structure and the selected T_{L} of interest into modeling software (including for example PyMOL, Glide, Maestro, RasMol, Visual Molecular Dynamics, Jrnol, and AutoDock) to determine what portion of the Extracellular Protein Targeting Ligand is bound to the Extracellular Protein. The bifunctional compound T_{L} is then bound to the linker (L_{I}) or sortilin binding group (S_{L}) at a point that does not unduly adversely affect binding to the extracellular protein.

### Non-Limiting Examples of Extracellular Proteins:

In one embodiment the extracellular target protein is one selected from the group consisting of: PCSK9, TNF-α, ANGPTL-3, an antibody light chain, IgG, IgE, IgA IL-1, IL-2, IL-6, IFN-γ, VEGF, TFG-β1, IL-21, IL-22, IL-5, IL-10, IL-8, cholinestearase, human CCL2, carboxypeptidase B-2, neutrophil elastase, Factor Xa, Factor XI, Factor XIa, Factor XII, Factor XIII, prothrombin, coagulation factor VII, coagulation factor IX, fibroblast growth factor 1, FGF-2, fibronectin 1, kallikrein-1, lipoprotein lipase, human matrix metallopeptidase 1, macrophage migration inhibitory factor, transformin growth factor-p (TGF-p), thrombospondin-1 (TSP-T), CD40 ligand, urokinase-type plasminogen activator, plasminogen activator tissue type (TPA), Plasminogen (PLG), Plasminogen Activator Inhibitor-1, Placenta Growth Factor, Phospholipase A2 Group IB, Phospholipase A2 Group IIA, Complement factor B, Complement factor D, complement factor H, Complement Component 5 and complement C1s.

### Immunoglobulin G (IgG)

In some embodiments, the Target Protein is a human immunoglobulin G (IgG). IgG represents approximately 75% of serum antibodies in humans. IgG is the most common type of antibody found in blood circulation. IgG antibodies are large globular proteins with a molecular weight of about 150 kDa made of four peptide chains. It contains two identical g (gamma) heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding site. The various regions and domains of a typical IgG are depicted in the figure to the left. The Fc regions of IgGs bear a highly conserved N-glycosylation site at asparagine 297 in the constant region of the heavy chain. The N-glycans attached to this site are predominantly core-fucosy fated biantennary structures of the complex type. In addition, small amounts of these N-glycans also bear bisecting GlcNAc and a-2,6-iinked sialic acid residues. The N-glycan composition in IgG has been linked to several autoimmune, infectious, and metabolic diseases. In addition, overexpression of IgG4 has been associated with IG4-related diseases, which generally include multiple organs, and disorders include type 1 autoimmune pancreatitis, interstitial nephritis, Riedel's thyroiditis, Mikulicz's disease, Kuttner's tumor, inflammatory' pseudotumors (in various sites of the body), mediastinal fibrosis and some cases of retroperitoneal fibrosis, aortitis, retroperitoneal fibrosis, proximal biliary strictures, tubulointerstitial nephritis, pachymeningitis, pancreatic enlargement and pericarditis.

The Protein Data Bank website provides the crystal structure of IgG searchable by 1H3X (Krapp, S., et al., J. Mol. Biol., 2003, 325:979); and 5V43 (Lee, C. H., et al., Nat. Immunol., 2017, 18:889-898); as well as the crystal structure of IgG bound to various compounds searchable by 5YC5 (Kiyoshi M., et ah, Sei. Rep., 2018, 8:3955-3955); 5XJE (Sakae Y., et al., Sci. Rep., 2017, 7:13780-13780); 5GSQ (Chen, C. L., et al., ACS Chem Biol, 2017, 12:1335-1345); and 1HZH (Saphire E. ( ), et. al, Science, 2001, 293: 1155-1159). Additionally, Kiyoshi, M., et al, provides insight into the structural basis for binding of human IgGI to its high-affinity human receptor FcyRI. (Kiyosi M., et al, Nat Commun., 2015, 6, 6866).

### TNF-Alpha

In some embodiments, the Target Protein is human TNF-α (UniProtKB—PC) 1375 (TNFA_HUMAN)). TNF-α is a pro-inflammatory cytokine active in the bodily immune response and serious inflammatory diseases. TNF-α has been implicated in several of disorders, including but not limited to rheumatoid arthritis, inflammatory bowel disease, graft-vs-host disease, ankylosing spondylitis, psoriasis, hidradenilis suppurativa, refractory asthma, systemic lupis erthyematosus, diabetes, and the induction of cachexia. As used herein, TNF-alpha may be referred to as TNFa, TNF-a, TNF-α or TNFa or TNFalpha.

The Protein Data Bank website provides the crystal structure of TNF-α searchable by 6RMJ (Valentinis, B., et al, Int. J. Mol. Sci., 2019, 20), 5UUI (Carrington et al., Biophys J., 2017, 113 371-380); 600Y, 600Z and 60PO (O'Connell, J., et al., Nat. Commun., 2019, 10 5795-5795), and 5TSVV (Cha, S. S., J Biol Che., 1998, 273 2153-2160); as well as the crystal structure of TNF-x bound to various compounds searchable by 5YOY (Ono et al., Protein Sci., 2018, 27 1038-1046); 2AZ5 (He., M. VI. et af, Science, 2005, 310:1022-1025); 5WUX (Lee, J. U., Int J Mol Sci., 2017, 18); 5MU8 (Blevitt et al., J Med Chem., 2017, 60 3511-3517); 4Y60 (Feldman J. I., et al., Biochemistry, 2015, 543037-3050); 3WD5 (Hu, S., et al., J Biol Chem, 2013, 28827059-27067); and 4G3Y (Liang, S. Y., J Biol Chem., 2013, 288 13799-13807).

### Proprotem Convertase Suhtilisin/Kexm Type 9 (PCSK-9)

In some embodiments, the Target Protein is human proprotein convertase subtilisin/kexin type 9 (PCSK-9) (UniProtKB—Q8NBP7 (PCSK9_HUMAN)). PCSK-9 is a crucial player in the regulation of plasma cholesterol homeostasis. PCSK-9 binds to low-density lipid receptor family members: low density lipoprotein receptor (LDLR), very low-density lipoprotein receptor (VLDLR), apolipoprotein E receptor (LRP1/APOER) and apolipoprotein receptor 2 (LRP8/APOER2), and promotes their degradation in intracellular acidic compartments. It acts via a non-proteoiytic mechanism to enhance the degradation of the hepatic LDLR through a clathrin LDLRAPI/ARH-mediated pathway, and may prevent the recycling of LDLR from endosomes to the cell surface or direct it to lysosomes for degradation. PCSK-9 has been implicated in high blood cholesterol and the development of cardiovascular disease.

The Protein Data Bank website provides the crystal structure of PCSK-9 searchable by 2P4E (Cunningham, D., et al., Nat Struct Mol Biol., 2007, 14 413-419); as well as the crystal structure of PCSK-9 bound to various compounds searchable by 3BPS (Kwon, H. J, et al, Proc Natl Acad Sei USA, 2008, 105 1820-1825); 6U26, 6U2N, 6U2P, 6U36, 6U38, and 6U3X (Petrilli, W. L., et al., Ceil Chem Biol., 2019, 27 32-40. e3); 50CA (Gustafsen, C., et al., Nat Commun., 2017, 8 503-503); 4NE9 (Schroeder, C. L, et al., Chem Biol., 2014, 21 284-294); 40V6 (Mitchell, T., et. al., J Pharmacol Exp Ther., 2014, 350412-424); and 4NMX (Zhang, Y., et. al., J Biol Chem., 2014, 289942-955). Additionally, Piper et al., provides insight into the crystal structure of PCSK9 (Piper, D. E., et ah, Structure, 2007, 15 (5), 545-52).

### TNFalpha

In one embodiment, the present disclosure provides for a bifunctional compound as described herein able to target TNFa. Thus, in one embodiment, the target molecules is TNFa.

In one embodiment, the targeting moiety (T_{L}) is according to formula XVIIa:

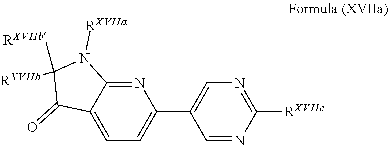

wherein, R^{XVIIa} has the formula

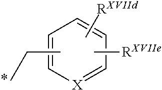

wherein R^{XVIId} and R^{XVIIe} are independently selected from the group consisting of C₁-C₅ alkyl, C₁-C₅ alkoxy, cyano, halogen and C₁ haloalkyl, each of which is optionally independently substituted; X is an atom selected from N or CH and * denotes the attachment with formula (XVIIa);
R^{XVIIb} and R^{XVIIb′} are each independently selected from H and a C₁-C₃ alkyl;
R^{XVIIc} is R_{L} or is selected from the group consisting of formula XVIIa-1 and formula XVIIa-2:

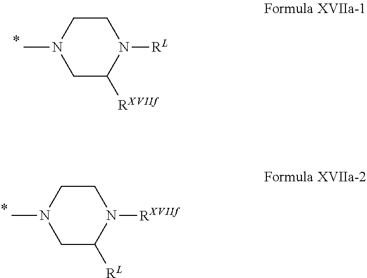

wherein R^{XVIIf} is selected from the group consisting of an optionally substituted C₁-C₅ alkyl wherein one or more of the methylene groups is substituted by a one selected from the group consisting of carbonyl, ester, amide, —NH— or —O—, and * denotes the attachment with formula XVIIa; and
R_{L} denotes the attachment with L_{I}.

In one embodiment, R^{XVIIb} and/or R^{XVIIb′} are —CH₃. In one embodiment, R^{XVIIb} and R^{XVIIb′} are —CH₃.

In one embodiment, R^{XVIIa} is selected from the group consisting of:

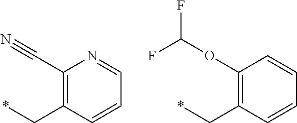

wherein * denotes the attachment with formula XVIIa.

In one embodiment, T_{L} is according to formula XVIIa-3:

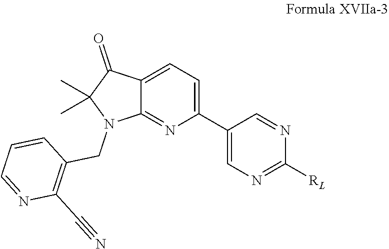

wherein R_{L} denotes attachment with L_{I}.

In one embodiment, T_{L} is according to formula XVIIa-4:

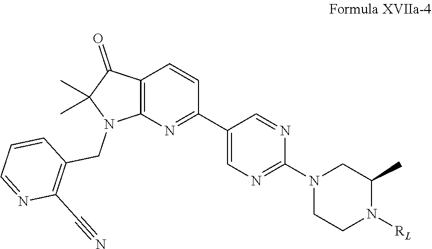

wherein R_{L} denotes attachment with L_{I}.

In one embodiment, T_{L} is according to formula XVIIa-5:

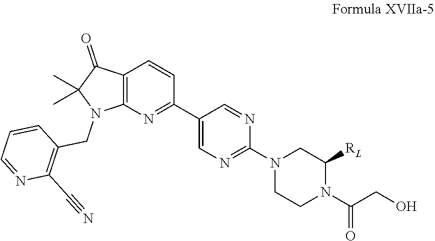

wherein R_{L} denotes attachment with L_{I}.

In one embodiment, T_{L} is according to formula XVIIb

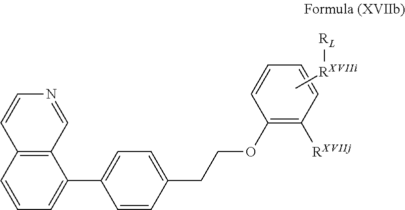

wherein R^{XVIIi} is selected from a bond and an optionally substituted piperazine group;
R^{XVIIj} is selected from the group consisting of

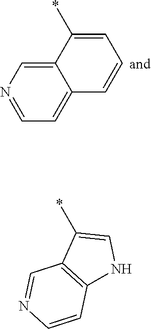 [SMILES: *c1cccc2ccncc12 ; *c1c[nH]c2ccncc12]

wherein * denotes attachment with formula XVIIb; and
wherein R_{L} denotes attachment with L_{I}.

In one embodiment, T_{L} is according to formula XVIIb-1:

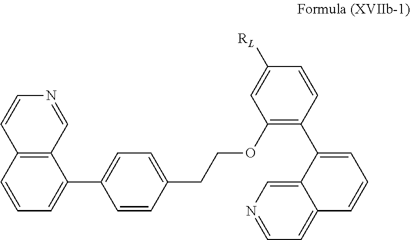

wherein R_{L} denotes attachment with L_{I}.

In one embodiment, T_{L} is according to formula XVIIb-2

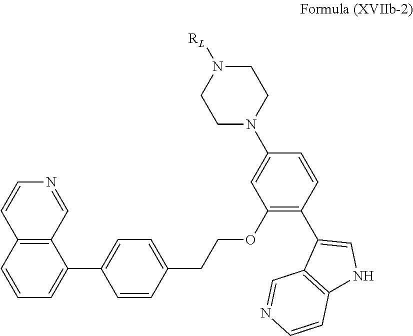

wherein R_{L} denotes attachment with L_{I}.

In one embodiment, T_{L} is according to formula XVIIc

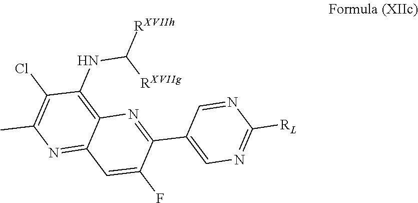

wherein R^{XVIIg} is a C₁-C₄ alkyl and R^{XVIIh} is an aromatic or heteroaromatic ring optionally substituted with one or more of the groups selected from halogen, haloalkyl, cyano, hydroxyl, amino, hydroxyl, alkoxy, C₃-C₆ cycloalkyl and C₃-C₆ heterocycloalkyl; and
wherein R_{L} denotes attachment with L_{I}.

In one embodiment, R^{XVIIg} is —CH₃.

In one embodiment, T_{L} is according to formula XVIIc-1

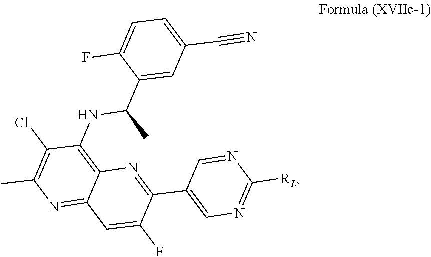

wherein R_{L} denotes attachment with L_{I}.

In one embodiment, T_{L} is according to formula XVIIc-2

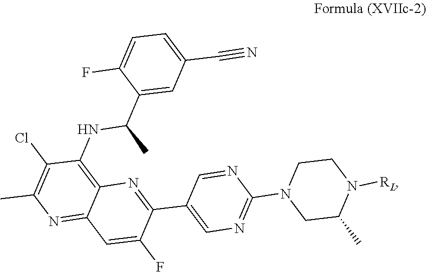

wherein R_{L} denotes attachment with L_{I}.

In one embodiment, T_{L} is according to formula XVIId

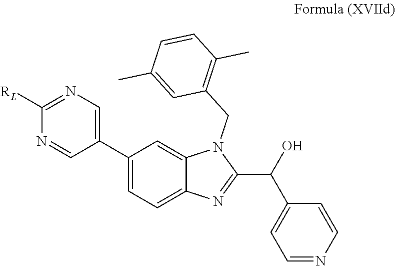

In aspect, the present disclosure provides for a compound selected from any one of the compounds TF001 to TF022 shown in Table C in the section “List of compounds”.

### TL Biotin and DNP

In one embodiment, the T_{L} moiety is a substituent derived from biotin. In another embodiment, T_{L} moiety has the following structure:

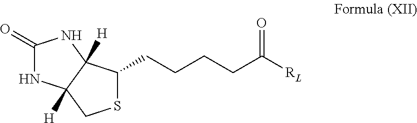

wherein R^{L} denotes the attachment with L_{I}.

A number of proteins bind strongly with biotin. Thus, in one embodiment, the extracellular target protein is a biotin-binding protein. For example, the biotin binding-protein may be selected from the group consisting of avidin, streptavidin or neutravidin or a derivative thereof. In some cases, the biotin binding protein is labelled with a tag moiety, such as a fluorophore, a chromophore, a radioactively labelled compound.

In one embodiment, the T_{L} moiety is a substituent derived from dinitrophenol (DNP). In another embodiment, the T_{L} moiety has the following structure:

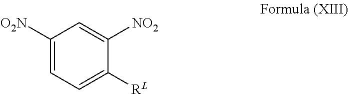

wherein R^{L} denotes the attachment with L_{I}.

In one embodiment, the extracellular target protein is a DNP binding-protein. For example, the extracellular target protein is an anti-DNP antibody, a fragment of an anti-DNP antibody able to bind DNP or a derivative thereof. In some cases, the anti-DNP antibody, or fragment thereof is labelled with a tag moiety, such as a fluorophore, a chromophore or a radioactively labelled compound.

### TL is a Protein

In some embodiments, T_{L} is an antibody, or an antigen-binding fragment of an antibody or a nanobody able to bind the extracellular target molecule. In one embodiment, T_{L} comprises an antibody, or an antigen-binding fragment of an antibody or a nanobody able to bind the extracellular target protein.

According to the present disclosure, T_{L} may comprise or consist of a full length antibody or an antibody fragment. In one embodiment T_{L} comprises or consist of an antibody fragment such as an antibody light chain (LC), an antibody heavy chain (HC), an antigen binding region (Fab), a variable region of a light chain (V_{L}) or a variable region of a heavy chain (V_{H}).

In one embodiment, T_{L} comprises or consist of an antibody heavy chain and/or an antibody light chain.

In one embodiment, T_{L} comprises or consists of a variable region of a light chain (V_{L}) of an antibody. In another embodiment, T_{L} comprises or consist of a variable region of a heavy chain (V_{H}) of an antibody.

In one embodiment T_{L} comprises or consists of a full length antibody. In one embodiment

T_{L} comprises or consists of a monoclonal antibody.

In one embodiment T_{L} comprises or consists of a fusion protein of two or more antibody fragments. Such as two antibody fragments, for example three antibody fragments, for example 4 antibody fragments, such as 5 antibody fragments, such as 6 antibody fragments, such as 7 antibody fragments, such as 8 antibody fragments.

For example, T_{L} comprises or consist of a single chain antigen binding region (scAb) or a single chain variable fragment (scFv). In one embodiment, T_{L} comprises a single chain variable fragment (scFv). A scFv is a fusion protein of the variable regions of the heavy chain (V_{H}) and of the variable region of the light chain (V_{L}) of an antibody in a single chain.

In one embodiment, T_{L} comprises or consists of a single chain antigen binding region (scAb). A scAb is a fusion protein of the variable regions of the light chain (V_{L}) and the heavy chain (V_{H}) together with one or more regions of the constant regions of either the heavy chain (C_{H}) or the light chain (C_{L}) of an antibody.

In one embodiment T_{L} comprises or consists of a nanobody. A nanobody (also known as single-domain antibody) is an antibody fragment consisting of a single monomeric variable heavy chain (V_{H}H) domain that selectively binds a specific antigen.

In one embodiment, T_{L} consists of an antibody or an antibody fragment or a fusion protein of antibody fragments as described herein.

Alirocumab is a monoclonal antibody that binds PCSK-9 approved for treatment of hypercholesterolemia. The heavy chain sequence of the alirocumab antibody is SEQ ID NO.: 66, the light chain is of SEQ ID NO.: 67.

In one embodiment, T_{L} comprises an antibody or an antigen-binding fragment thereof with binding specificity to PCSK-9 comprising:
a light chain region comprising SEQ ID NO.: 67 or an amino acid sequence having at least 70% sequence identity to SEQ ID NO.: 67, for example at least 80%, 85%, 90%, 95%, 98% or 99% sequence identity;
and/or
a heavy chain region comprising SEQ ID NO.: 66.
or an amino acid sequence having at least 70% sequence identity to SEQ ID NO.: 66, for example at least 80%, 85%, 90%, 95%, 98% or 99% sequence identity.

In one embodiment, T_{L} comprises or consists of an antibody or an antigen-binding fragment thereof with binding specificity to PCSK-9 comprising or consisting of:
a light chain region comprising SEQ ID NO.: 67
and/or
a heavy chain region comprising SEQ ID NO.: 66.

In one embodiment, T_{L} comprises of consists of the full length monoclonal antibody Alirocumab (antibody with light chain according to SEQ ID NO.: 67 and heavy chain according to SEQ ID NO.: 66)

Adalimumab is a monoclonal antibody that binds TNF-α The heavy chain sequence of Adalimumab is of SEQ ID NO.: 68, the light chain sequence is of SEQ ID NO.: 69

In one embodiment, T_{L} comprises an antibody or an antigen-binding fragment thereof with binding specificity to TNF-α comprising:
a light chain region comprising SEQ ID NO.: 69 or an amino acid sequence having at least 70% sequence identity to SEQ ID NO.: 69, for example at least 80%, 85%, 90%, 95%, 98% or 99% sequence identity;
and/or
a heavy chain region comprising SEQ ID NO.: 68.
or an amino acid sequence having at least 70% sequence identity to SEQ ID NO.: 68, for example at least 80%, 85%, 90%, 95%, 98% or 99% sequence identity.

In one embodiment, T_{L} comprises or consists of an antibody or an antigen-binding fragment thereof with binding specificity to TNF-α comprising or consisting of:
a light chain region comprising SEQ ID NO.: 69
and/or
a heavy chain region comprising SEQ ID NO.: 68.

In one embodiment, T_{L} comprises of consists of the full length monoclonal antibody Adalimumab.

In one embodiment, T_{L} comprises a nanobody with binding specificity to the kappa-light chain of a second antibody comprising the following sequence:

(SEQ ID NO.: 70)
MGGTHHHHHHENLYFQGQVQLQESGGGLVQPGGSLRLSCAASGRTISRY
AMSWFRQAPGKEREFVAVARRSGDGAFYADSVQGRFTVSRDDAKNTVYL
QMNSLKPEDTAVYYCAIDSDTFYSGSYDYWGQGTQVTVSSE

or an amino acid sequence having at least 70% sequence identity to SEQ ID NO.: 70, for example at least 80%, 85%, 90%, 95%, 98% or 99% sequence identity.

In one embodiment, T_{L} comprises or consists of the following sequence:

SEQ ID NO.: 70
MGGTHHHHHHENLYFQGQVQLQESGGGLVQPGGSLRLSCAASGRTISRY
AMSWFRQAPGKEREFVAVARRSGDGAFYADSVQGRFTVSRDDAKNTVYL
QMNSLKPEDTAVYYCAIDSDTFYSGSYDYWGQGTQVTVSSE

In one embodiment, the bifunctional compound according to the present disclosure is able to bind the target molecule with a dissociation constant (K_{D}) of less than 50 μM, such as less than 40 μM, such as less than 30 μM, such as less than 20 μM, such as less than 10 μM, such as less than 5 μM, such as less than 4 μM, such as less than 3 μM, such as less than 2 μM, such as less than 1 μM, such as less than 0.8 μM, such as less than 0.6 μM, such as less than 0.5 μM, such as less than 0.4 μM, such as less than 0.3 M, such as less than 0.2 μM, such as less than 0.1 μM.

The binding with the target protein may be measured through different methods as it is known to someone of skill in the art. For example, microscale thermophoresis (MST),

In one embodiment, the bifunctional compound is according to any one of formulas XVIIIa, XVIIIb, XVIIIb-1, XVIIIc, XVIIId, XVIIId-1, XVIIIe, XVIIIf, XVIIIf-1, XVIIIg, XVIIIh and XVIIIh-1:

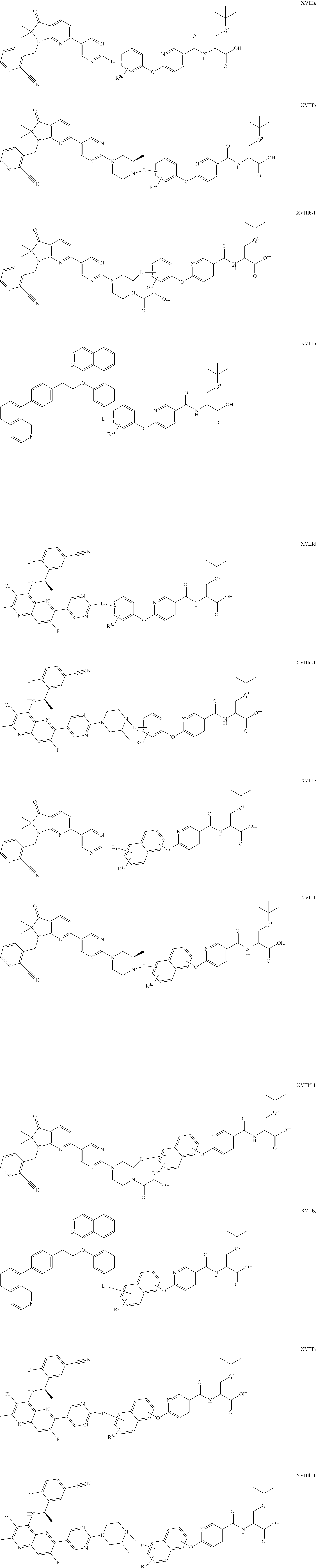

wherein
Q³ is a bond or —CH₂—;
R^{3a} is selected from the group consisting of H, halogen, alkoxy, —CF₃, and an optionally substituted C₁-₄ alkyl; and
L_{I} denotes the linker.

In one embodiment, R^{3a} is H.

In one embodiment, R^{3a} is halogen or —CF₃.

In one embodiment, R^{3a} is alkoxy.

In one embodiment, R^{3a} is an optionally substituted C₁-₄ alkyl.

In one embodiment, Q³ is a bond.

In one embodiment, Q³ is —CH₂—.

In one embodiment, the bifunctional compound is according to any one of formulas D-III, D-IV, D-V and D-VI:

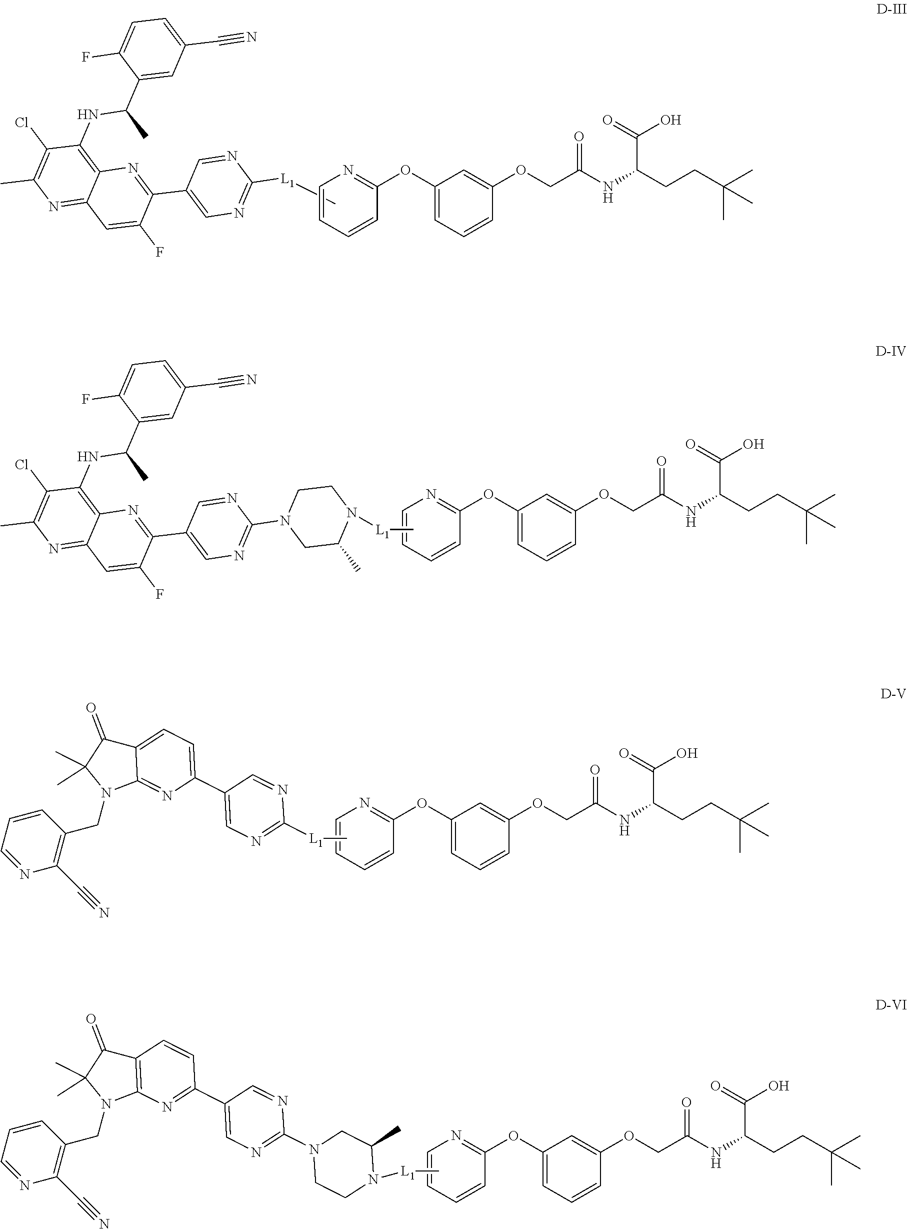

In one embodiment, the bifunctional compound is according to any one of formulas XVIIIm, XVIIIn, XVIIIn-1, XVIIIo, XVIIIp:

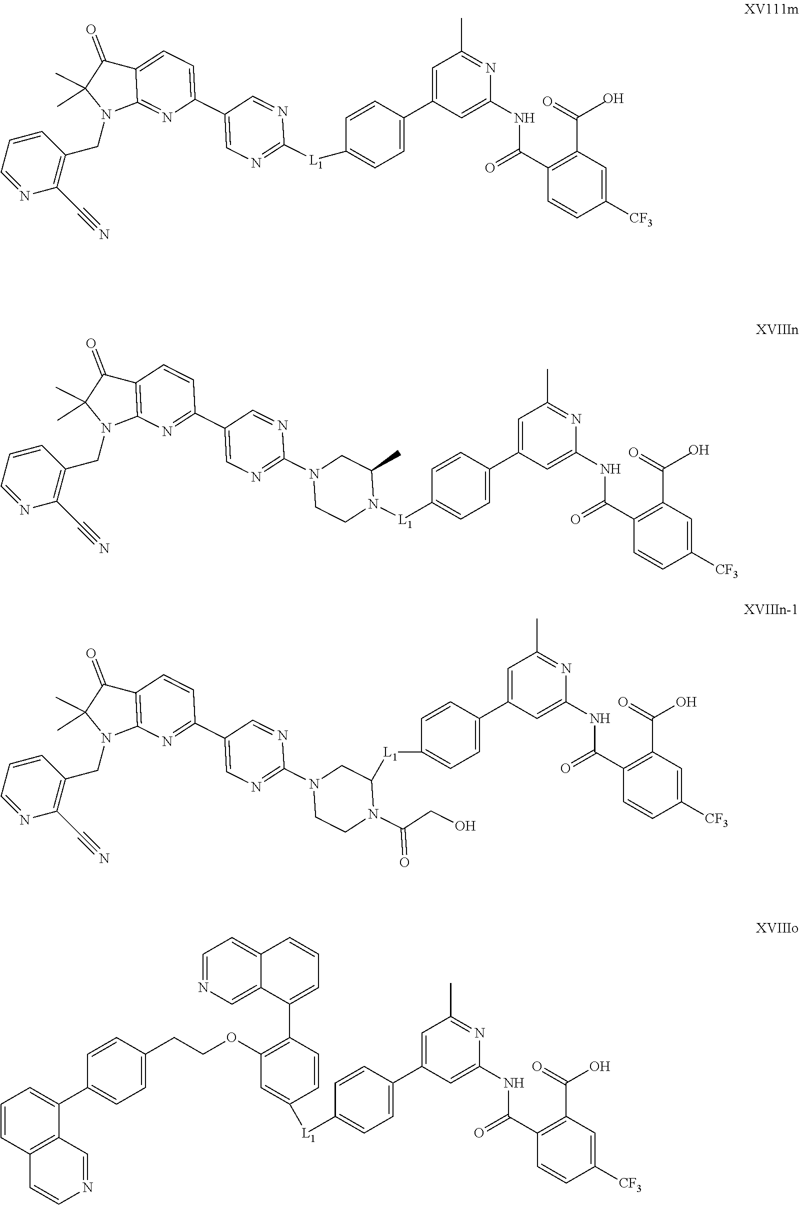

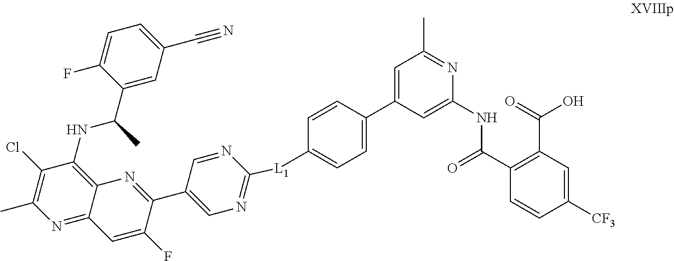

wherein L_{I} denotes the linker.

In one embodiment, the bifunctional compound is according to any one of formulas XIVa, XIVb, XIVc and XIVd:

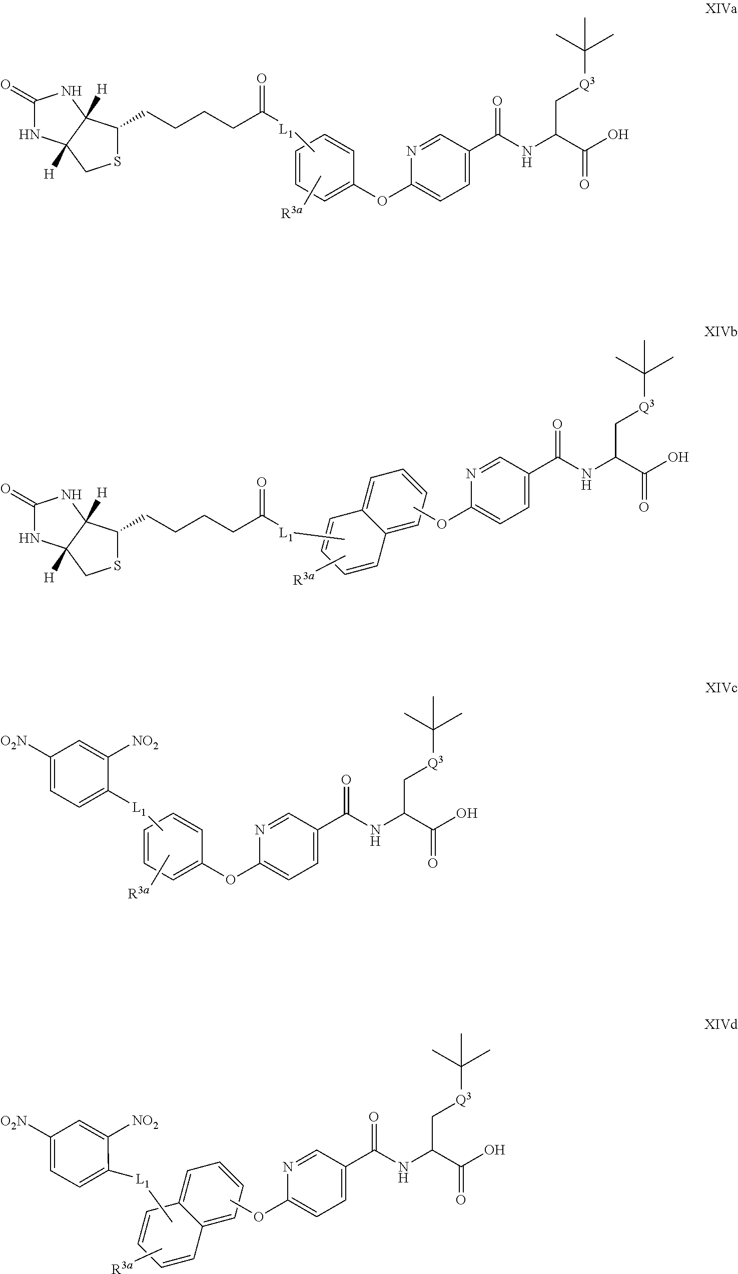

wherein
Q³ is a bond or —CH₂—;
R^{3a} is selected from the group consisting of H, halogen, alkoxy, —CF₃, and an optionally substituted C₁-₄ alkyl.

In one embodiment, in any one of formulas XIVa, XIVb, XIVc or XIVd R^{3a} is H.

In one embodiment, in any one of formulas XIVa, XIVb, XIVc or XIVd R^{3a} is halogen or —CF₃.

In one embodiment, in any one of formulas XIVa, XIVb, XIVc or XIVd R^{3a} is alkoxy.

In one embodiment, in any one of formulas XIVa, XIVb, XIVc or XIVd R^{3a} is an optionally substituted C₁-₄ alkyl.

In one embodiment, in any one of formulas XIVa, XIVb, XIVc or XIVd Q³ is a bond.

In one embodiment, in any one of formulas XIVa, XIVb, XIVc or XIVd Q³ is —CH₂—.

In one embodiment, the bifunctional compound is according to any one of formulas XVa, XVb, XVc and XVd:

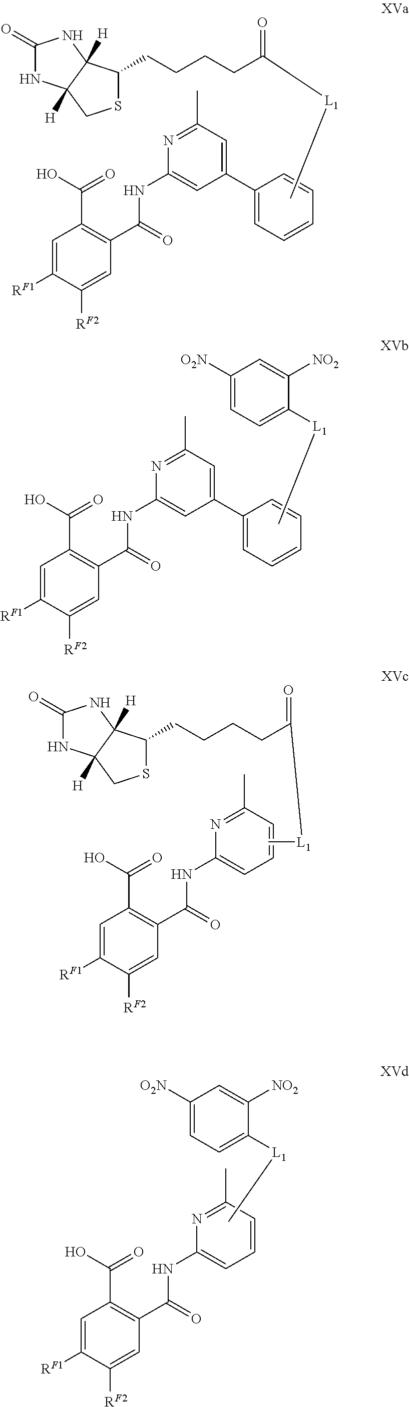

wherein,
R^{F1} and R^{F2} are independently selected from CF₃ or H, with the proviso that at least one of R^{F1} or R^{F2} is CF₃.

In one embodiment, in any one of formulas XIVa, XIVb, XIVc or XIVd R^{F1} is —CF₃ and R^{F2} is H.

In one embodiment, the bifunctional compound is according to one selected from the group consisting of:

(SEQ ID NO.: 72)
X_{T}-X_{L}-RQLL-OH,
(SEQ ID NO.: 73)
X_{T}-X_{L}-FQLL-OH,
(SEQ ID NO.: 74)
X_{T}-X_{L}-KQLL-OH,
(SEQ ID NO.: 75)
X_{T}-X_{L}-PQLL-OH,
(SEQ ID NO.: 76)
X_{T}-X_{L}-PYIL-OH,
(SEQ ID NO.: 77)
X_{T}-X_{L}-RYLL-OH,
(SEQ ID NO.: 78)
X_{T}-X_{L}-FYLL-OH,
(SEQ ID NO.: 79)
X_{T}-X_{L}-YQLL-OH,
(SEQ ID NO.: 80)
X_{T}-X_{L}-LLQL-OH,
(SEQ ID NO.: 81)
X_{T}-X_{L}-FYIL-OH,
(SEQ ID NO.: 82)
X_{T}-X_{L}-RYIL-OH,
(SEQ ID NO.: 83)
X_{T}-X_{L}-PYLL-OH,
(SEQ ID NO.: 84)
X_{T}-X_{L}-PYYL-OH,
(SEQ ID NO.: 85)
X_{T}-X_{L}-PYFL-OH,
(SEQ ID NO.: 86)
X_{T}-X_{L}-RYYL-OH,
(SEQ ID NO.: 87)
X_{T}-X_{L}-RYFL-OH,
(SEQ ID NO.: 88)
X_{T}-X_{L}-RQFL-OH,
(SEQ ID NO.: 89)
X_{T}-X_{L}-RQYL-OH,
(SEQ ID NO.: 90)
X_{T}-X_{L}-LQLL-OH,
(SEQ ID NO.: 91)
X_{T}-X_{L}-HQLL-OH,
(SEQ ID NO.: 92)
X_{T}-X_{L}-IQLL-OH,
(SEQ ID NO.: 93)
X_{T}-X_{L}-FYYL-OH,
(SEQ ID NO.: 94)
X_{T}-X_{L}-PYMKLAPGELTIIL-OH,
(SEQ ID NO.: 95)
X_{T}-X_{L}-NEKLSQLQTYMI-OH,
(SEQ ID NO.: 96)
X_{T}-X_{L}-KDADLYTSRVMLSSQVP-OH,
(SEQ ID NO.: 99)
X_{T}-X_{L}-ITVDPRLFKKRRLRSPRVLFSTQPPR-OH,
(SEQ ID NO.: 100)
X_{T}-X_{L}-WSGPIGVSWGLRAAAAGGAFP-OH,
(SEQ ID NO.: 101)
X_{T}-X_{L}-WSGPIGVSWGLRAAAAGGAFPRGGRWRR-OH,
(SEQ ID NO.: 102)
X_{T}-X_{L}-GVSWGLR-OH,
(SEQ ID NO.: 103)
X_{T}-X_{L}-REAPRWDAPLRDPALRQLL-OH,
(SEQ ID NO.: 104)
X_{T}-X_{L}-REALRWDAPLRDPAPRQLL-OH,
(SEQ ID NO.: 105)
X_{T}-X_{L}-PYILKRQLYENKPRRPYIL-OH,
(SEQ ID NO.: 106)
X_{T}-X_{L}-LYENKPRRPYIL-OH,
(SEQ ID NO.: 107)
X_{T}-X_{L}-APLRDPAPRQLL-OH,
(SEQ ID NO.: 108)
X_{T}-X_{L}-REAPRWDAPLRDPALFQLL-OH,
(SEQ ID NO.: 109)
X_{T}-X_{L}-REAPRWDAPLRDPALRYLL-OH,
(SEQ ID NO.: 110)
X_{T}-X_{L}-REAPRWDAPLRDPALRQYL-OH,
(SEQ ID NO.: 111)
X_{T}-X_{L}-REAPRWDAPLRDPALRYYL-OH,
(SEQ ID NO.: 112)
X_{T}-X_{L}-TGGFM-OH,
and
(SEQ ID NO.: 113)
X_{T}-X_{L}-YGGFL-OH;

wherein X_{L} is L_{I} and X_{T} is selected from the group consisting of:
a) a group comprising biotin;
b) a group comprising dinitrophenol;
c) an antibody with binding specificity to PCSK-9 comprising or consisting of:
   a light chain region comprising SEQ ID NO.: 67
   and/or
   a heavy chain region comprising SEQ ID NO.: 66
   or an antigen binding fragment thereof.
d) an antibody with binding specificity to TNF-α comprising or consisting of:
   a light chain region comprising SEQ ID NO.: 69
   and/or
   a heavy chain region comprising SEQ ID NO.: 68
   or an antigen binding fragment thereof.

In one embodiment, the bifunctional compound is according to:

(SEQ ID NO.: 116)
Biotin-X_{L}-X₅-X₁X₂X₃X₄

wherein,
X₅ is an optional amino acid residue or peptide comprising 2 to 30 amino acid residues or a bond,
X₁ is R, P, F, Y, L, K, G or H;
X₂ is Q, Y, L, E or G;
X₃ is Y, F, L, I, Q, E or N;
X₄ is M, K, L or a conservative substitution of L;
wherein X_{L} is L_{I}.

In one embodiment, X₅ is a bond.

In one embodiment, X₅ is X₅ is APLRDPAP (SEQ ID NO.: 21).

In one embodiment, X₅ is REAPRWDAPLRDPAL (SEQ ID NO.: 17).

In one embodiment, X₅ is REALRWDAPLRDPAP (SEQ ID NO.: 18).

In one embodiment,
X₁ is selected from Y, F or R,
X₂ is selected from Q or Y,
X₃ is L,
X₄ is L.

In one embodiment, X₁ is F or R.

In one embodiment, X₂ is Q.

In one embodiment, X₃ is Q.

In one embodiment, X₄ is L.

In one embodiment, the bifunctional compound is according to any one of the group consisting of:

(SEQ ID NO.: 117)
Biotin-X_{L}-RQLL-OH
(SEQ ID NO.: 118)
Biotin-X_{L}-FQLL-OH
(SEQ ID NO.: 119)
Biotin-X_{L}-RYLL-OH
(SEQ ID NO.: 120)
Biotin-X_{L}-FYLL-OH
(SEQ ID NO.: 121)
Biotin-X_{L}-YQLL-OH
(SEQ ID NO.: 122)
Biotin-X_{L}-REAPRWDAPLRDPALRQLL-OH
(SEQ ID NO.: 123)
Biotin-X_{L}-REALRWDAPLRDPAPRQLL-OH
(SEQ ID NO.: 124)
Biotin-X_{L}-REAPRWDAPLRDPALFQLL-OH
(SEQ ID NO.: 125)
Biotin-X_{L}-REAPRWDAPLRDPALRYLL-OH
(SEQ ID NO.: 126)
Biotin-X_{L}-REAPRWDAPLRDPALRQYL-OH
(SEQ ID NO.: 127)
Biotin-X_{L}-REAPRWDAPLRDPALRYYL-OH
(SEQ ID NO.: 128)
Biotin-X_{L}-APLRDPAPRQLL-OH
(SEQ ID NO.: 129)
Biotin-XL-PYILKRQLYENKPRRPYIL-OH
(SEQ ID NO.: 130)
Biotin-X_{L}-LYENKPRRPYIL-OH

wherein X_{L} is L_{I}.

### Antibody Conjugates

In one embodiment the bifunctional compound is prepared by forming a conjugate between a monoclonal antibody and a reactive precursor of the group-L_{I}-S_{L}. For example, the group-L_{I}-S_{L} may be conjugated to the free amino groups in the antibody sequence, such as the side chains of lysine amino acids or the N-terminal group. Multiple methods and conditions are well known in the art to conjugate groups to the free amino groups of an antibody. For example, an N-hydroxy succinimide (NHS) derivative of L_{I}-S_{L} may be prepared and reacted with the antibody.

Thus in one embodiment, the bifunctional compound is prepared by reacting a NHS derivative of -L_{I}-S_{L} with a monoclonal antibody.

As it will be understood by a person skilled in the art, reaction of an NHS derivative of -L_{I}-S_{L} with a monoclonal antibody will yield a product wherein more than one units of -L_{I}-S_{L} may be conjugated to the antibody.

Thus, in one embodiment, the bifunctional compound has a T_{L} group consisting of a monoclonal antibody conjugated to at least 1-L_{I}-S_{L} unit, for example to 2-L_{I}-S_{L} units, for example to 3-L_{I}-S_{L} units, for example to 4-L_{I}-S_{L} units, for example to 5-L_{I}-S_{L} units, for example to 6-L_{I}-S_{L} units, for example to 7-L_{I}-S_{L} units, for example to 8-L_{I}-S_{L} units, for example to 9-L_{I}-S_{L} units.

In one embodiment, the monoclonal antibody is alirocumab (antibody having heavy chain SEQ ID NO.: 66 and light chain SEQ ID NO.: 67) and S_{L} and/or L_{I} is as described herein.

In one embodiment, the monoclonal antibody is alirocumab (antibody having heavy chain SEQ ID NO.: 66 and light chain SEQ ID NO.: 67) and S_{L} a peptide comprising a sequence at the C-terminus selected from the group consisting of:

(SEQ ID NO.: 22)
RQLL-OH
(SEQ ID NO.: 23)
FQLL-OH
(SEQ ID NO.: 27)
RYLL-OH
(SEQ ID NO.: 28)
FYLL-OH
(SEQ ID NO.: 29)
YQLL-OH
and
(SEQ ID NO.: 55)
PYILKRQLYENKPRRPYIL-OH;

wherein L_{I} is attached at the N-terminus.

In one embodiment, the monoclonal antibody is alirocumab (antibody having heavy chain SEQ ID NO.: 66 and light chain SEQ ID NO.: 67) and S_{L} is a peptide comprising a the following sequence at the C-terminus: RQLL-OH (SEQ ID NO.: 22).

In one embodiment, the monoclonal antibody is alirocumab (antibody having heavy chain SEQ ID NO.: 66 and light chain SEQ ID NO.: 67) and S_{L} is a peptide comprising a the following sequence at the C-terminus: FQLL-OH (SEQ ID NO.: 23).

In one embodiment, the monoclonal antibody is adalimumab (antibody having heavy chain SEQ ID NO.: 68 and light chain SEQ ID NO.: 69) and S_{L} is as described herein.

In one embodiment, the monoclonal antibody is adalimumab (antibody having heavy chain SEQ ID NO.: 68 and light chain SEQ ID NO.: 69) and S_{L} a peptide comprising a sequence at the C-terminus selected from the group consisting of:
RQLL-OH (SEQ ID NO.: 22); FQLL-OH (SEQ ID NO.: 23); RYLL-OH (SEQ ID NO.: 27); FYYL-OH (SEQ ID NO.: 28); YQLL-OH (SEQ ID NO.: 29) and PYILKRQLYENKPRRPYIL-OH (SEQ ID NO.: 55);
wherein L_{I} is attached at the N-terminus.

In one embodiment, the monoclonal antibody is adalimumab (antibody having heavy chain SEQ ID NO.: 68 and light chain SEQ ID NO.: 69) and S_{L} a peptide comprising a the following sequence at the C-terminus: RQLL-OH (SEQ ID NO.: 22).

In one embodiment, the monoclonal antibody is adalimumab (antibody having heavy chain SEQ ID NO.: 68 and light chain SEQ ID NO.: 69) and S_{L} a peptide comprising a the following sequence at the C-terminus: FQLL-OH (SEQ ID NO.: 23).

### Linkers:

The bifunctional compounds according to the present disclosure comprise a linker that joins covalently the sortilin binding moiety (S_{L}) and the targeting moiety (T_{L}). Covalent conjugation of the S_{L} and T_{L} groups through a linker to form a bifunctional compound can be done through multiple strategies chemical strategies as known in the art.

In one embodiment, the bifunctional compound according to the present disclosure has L_{I} according to the following structure:

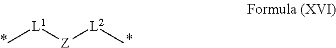

wherein
* denotes the point of attachment to either T_{L} or S_{L};
   L₁ and L₂ are each independently selected from the group consisting of a bond, —C(H₂)—, —O—, —N(H)—; a functional group selected from carbonyl, ester, amide, carbamate, thiourea, urea, sulphonamide and triazole; or a C₁-C₃ hydrocarbon chain wherein one or more methylene groups are individually and optionally replaced with a carbonyl, ester, amide, carbamate, thiourea, urea sulphonamide and triazole;
Z is selected from the group consisting of: a bivalent, saturated or unsaturated, straight or branched, C₁-C₃₀ hydrocarbon chain wherein one or more methylene groups are individually and optionally replaced by one or more of the groups selected from: —O—, —N(H)—, —N(R^{L1})—, —OC(═O)—, —C(═O)O—, —C(═O)—, —N(H)C(═O)—, —N(R^{L1})C(═O)—, —C(═O)N(H)—, —NHC(═O)NH—, —NHC(═O)O—C(═O)N(R^{L1})—, —S—, —S(═O)—, —S(═O)₂—, —N(R^{L1})S(═O)₂—, —S(═O)₂N(R^{L1})—; an optionally substituted bivalent aromatic group; an optionally substituted carbocycle; an optionally substituted heterocycle; an optionally substituted bivalent aromatic heterocycle;

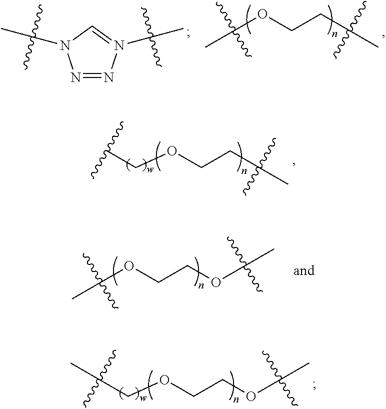

[symbol] denotes the attachment to L¹ or L²;
R^{L1} is selected from the group consisting of C₁₋₅ alkyl;
n and w each individually integers from 1 to 8.

In one embodiment, the bifunctional compound according to the present disclosure has L_{I} according to formula (XVI-2), wherein:

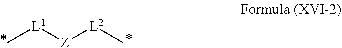

wherein
* denotes the point of attachment to either T_{L} or S_{L},
   L¹ and L² are each independently selected from the group consisting of —C(H₂)—, —O—, —N(H)—, and an amide;
Z is a bivalent, saturated or unsaturated, straight or branched, C₁-C₃₀ hydrocarbon chain wherein one or more methylene groups are individually and optionally replaced by one or more of the groups selected from: —O—, —N(H), —N(R^{L1})—, —N(H)C(═O)—, —N(R)C(═O)—, —C(═O)N(H)—, —C(═O)N(R^{L1})—,

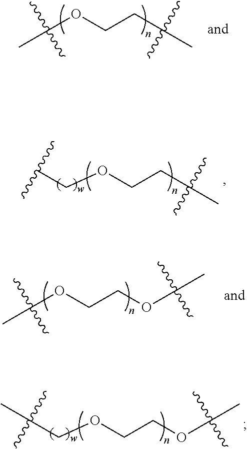

[symbol] denotes the attachment to L¹ or L²;
R^{L1} is selected from the group consisting of C₁₋₅ alkyl;
n and w each individually integers from 1 to 8.

In one embodiment, Z is a bivalent C₁-C₃₀ hydrocarbon chain, such as C₅-C₃₀ hydrocarbon chain, such as a C₈-C₃₀ hydrocarbon chain, such as a C₁₀-C₃₀ hydrocarbon chain, such as a C₁₂-C₃₀ hydrocarbon chain wherein one or more methylene groups are replaced as described in formula XVI or formula XVI-2.

In one embodiment, Z is a bivalent C₁₀-C₂₅ hydrocarbon chain, such as a C₁₀, C₁, C₁₂, C₁₃, C₁₄, C₁₅, C₁₆, C₁₇, C₁₈, C₁₉, C₂₀, C₂₁, C₂₂, C₂₃, C₂₄ or C₂₅ hydrocarbon chain, wherein wherein one or more methylene groups are replaced as described in formula XVI or formula XVI-2.

In one embodiment, Z is a bivalent C₁₄-C₂₀ hydrocarbon chain wherein one or more methylene groups are replaced as described in formula XVI or formula XVI-2. In one embodiment, Z is a bivalent C₇-C₁₃ hydrocarbon chain wherein one or more methylene groups are replaced as described in formula XVI or formula XVI-2.

In one embodiment, L¹ is a bond. In one embodiment L² is a bond. In one embodiment L¹ and L² are bonds.

In one embodiment, Z is a C₁-C₃₀ hydrocarbon chain wherein one or more methylene groups are individually and optionally replaced by one or more of the groups selected from: —O—, —N(H), —N(H)—C(═O)—, —C(═O)—N(H)—, urea, a triazole, an optionally substituted carbocycle; an optionally substituted heterocycle, and —CH₂—CH₂—O—.

In one embodiment, Z comprises a C₁-C₃₀ hydrocarbon chain, such as a C₁-C₂₀, such as a C₁-C₁₅, such as a C₁-C₁₀ hydrocarbon chain; wherein one or more methylene groups are individually and optionally replaced by one or more of the groups selected from: —O—, —N (H)—, —N(R^{L1})—, —OC(═O)—, —C(═O)O—, —C(═O)—, —N(H)C(═O)—, —NHC(═O)NH—, —NHC(═O)O—, —N(R^{L1})C(═O)—, —C(═O)N(H)—, —C(═O)N(R^{L1})—, —S—, —S(═O)—, —S(═O)₂—, —N(R^{L1})S(═O)₂—, —S(═O)₂N(R^{L1})—, —CH₂—CH₂—O—, an optionally substituted carbocycle; an optionally substituted heterocycle, and a triazole; R^{L1} is selected from the group consisting of C₁₋₅ alkyl.

In one embodiment, one or more methylene groups, such as 1, 2, 3, 4, 5, 6, 7, 8, 9 or 10 methylene groups of the hydrocarbon chain in Z are individually and optionally replaced by one or more of the groups selected from: —O—, —N(H)—, —N(R^{L1})—, —OC(═O)—, —C(═O)O—, —C(═O)—, —N(H)C(═O)—, —N(R^{L1})C(═O)—, —C(═O)N(H)—, —C(═O)N(R^{L1})—, —S—, —S(═O)—, —S(═O)₂—, —N(R^{L1})S(═O)₂—, —S(═O)₂N(R^{L1})—, —CH₂—CH₂—O—, an optionally substituted carbocycle; an optionally substituted heterocycle, and a triazole; R^{L1} is selected from the group consisting of C₁₋₅ alkyl.

In one embodiment, Z comprises one or more groups —NH—SO²-groups.

In one embodiment, Z comprises one or more triazole groups. In one embodiment, triazole refers to

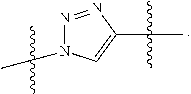 [SMILES: *c1cn(*)nn1]

In one embodiment, Z comprises one or more groups selected from: an optionally substituted carbocycle group(s) and an optionally substituted heterocycle group(s). In one embodiment, Z comprises two groups each individually selected from: a triazole, an optionally substituted carbocycle group(s) and an optionally substituted heterocycle group(s). In one embodiment, Z comprises three groups each individually selected from: a triazole, an optionally substituted carbocycle group(s) and an optionally substituted heterocycle group(s).

Non limiting examples of carbocycles, heterocycles are described herein below. In one embodiment, the one or more carbocyle or heterocycle is as described herein below.

In one embodiment, Z comprises a carbocyle according to

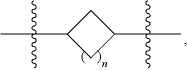

wherein n is an integer selected from 0, 1, 2 or 3.

In one embodiment, Z comprises one or more heterocycle groups. For example, the heterocycle group may be an optionally substituted 3 to 6 membered ring wherein one or two carbon atoms of the ring have been replaced by N. In one embodiment, Z comprises a heterocycle group is according to

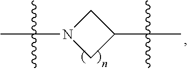

wherein n is an integer selected from 0, 1, 2 or 3.

In one embodiment, Z comprises one or more groups, each individually selected from the group consisting of shown in Table Z:

TABLE Z

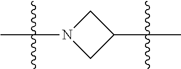 [SMILES: *C1CN(*)C1]

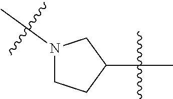 [SMILES: *C1CCN(*)C1]

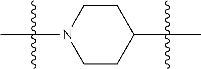 [SMILES: *C1CCN(*)CC1]

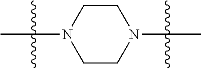 [SMILES: *N1CCN(*)CC1]

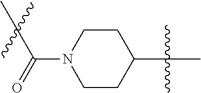 [SMILES: *C(=O)N1CCC(*)CC1]

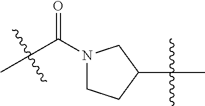 [SMILES: *C(=O)N1CCC(*)C1]

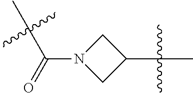 [SMILES: *C(=O)N1CC(*)C1]

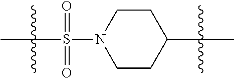 [SMILES: *C1CCN(S(*)(=O)=O)CC1]

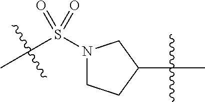 [SMILES: *C1CCN(S(*)(=O)=O)C1]

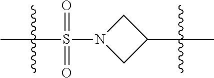 [SMILES: *C1CN(S(*)(=O)=O)C1]

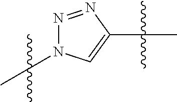 [SMILES: *c1cn(*)nn1]

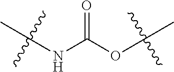 [SMILES: *NC(=O)O*]

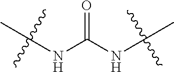 [SMILES: *NC(=O)N*]

In one embodiment, Z comprises two groups each individually selected from the groups shown in table Z. In one embodiment, Z comprises three groups each individually selected from the groups shown in table Z.

In one embodiment, Z comprises one or more groups each individually selected from the groups shown in table Z—I:

TABLE Z-I

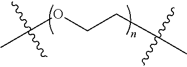

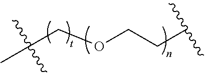

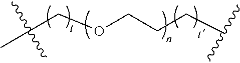

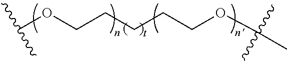

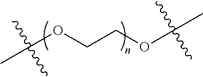

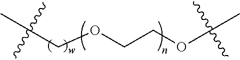

wherein n and/or n′ are each individually integers from 1 to 10 and t, t′ and/or w are each individually integers from 1 to 20.

In one embodiment, Z comprises

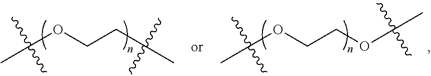

wherein n is an integer from 1 to 10.

In one embodiment, Z comprises

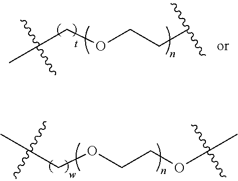

wherein n is an an integer from 1 to 10 and t or w is an integer from 1 to 20.

In one embodiment, Z comprises

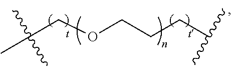

wherein n is an integer from 1 to 10 and each of t and t′ is individually an integer from 1 to 20.

In one embodiment, Z comprises

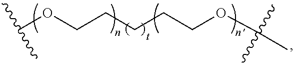

wherein each of n and n′ is individually an integer from 1 to 10 and t is an integer from 1 to 20.

In one embodiment, L¹ or L² are an amide group. In one embodiment, L¹ and L² are an amide group.

In one embodiment, L¹ or L² are a carbonyl group. In one embodiment, L¹ and L² are a carbonyl group.

In one embodiment, L¹ or L² are an ester group. In one embodiment, L¹ and L² are an ester group.

In one embodiment, L¹ or L² are a carbamate group. In one embodiment, L¹ and L² are a carbamate group.

In one embodiment, L¹ or L² are an urea group. In one embodiment, L¹ and L² are an urea group.

In one embodiment, L¹ or L² are —NH—S(═O)₂—. In one embodiment, L¹ and L² are —NH—S(═O)₂—.

In one embodiment, L¹ or L² are a triazole group. In one embodiment, L¹ and L² are a triazole group. A triazole group may be prepared via the reaction of an azide group with an alkyne group, optionally in the presence of a catalyst, for example using copper ions as catalyst.

In one embodiment, L¹ or L² are —O—. In one embodiment, L¹ and L² are —O—.

In one embodiment, L¹ or L² are —NH—. In one embodiment, L¹ and L² are —NH—.

In one embodiment, L¹ or L² are a —S(═O)₂—. In one embodiment, L¹ and L² are a —S(═O)₂—.

In one embodiment, L¹ or L² are

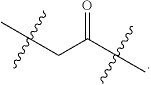 [SMILES: *CC(*)=O]

In one embodiment, L¹ and L² are

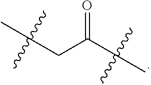 [SMILES: *CC(*)=O]

In one embodiment, L¹ or L² are

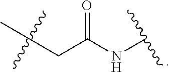 [SMILES: *CC(=O)N*]

In one embodiment, L¹ and L² are

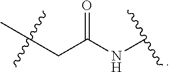 [SMILES: *CC(=O)N*]

In one embodiment, L¹ or L² are

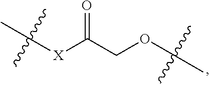

wherein X is NH or O. In one embodiment, L¹ and L² are

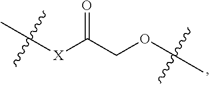

wherein X is NH or O. In one embodiment, L¹ or L² are

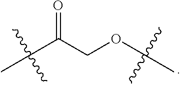 [SMILES: *OCC(*)=O]

In one embodiment, L¹ and L² are

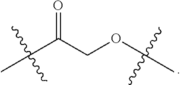 [SMILES: *OCC(*)=O]

In one embodiment, L¹ or L² are

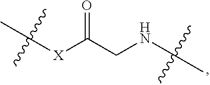

wherein X is NH or O. In one embodiment, L¹ and L² are

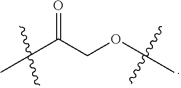

wherein X is NH or O. In one embodiment, L¹ or L² are

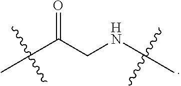 [SMILES: *NCC(*)=O]

In one embodiment, L¹ and L² are

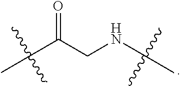 [SMILES: *NCC(*)=O]

In one embodiment, L¹ and L² are different groups. In one embodiment, L¹ and L² are identical.

In one embodiment, the bifunctional compound according to the present disclosure has L_{I} according to one selected from the group consisting of:

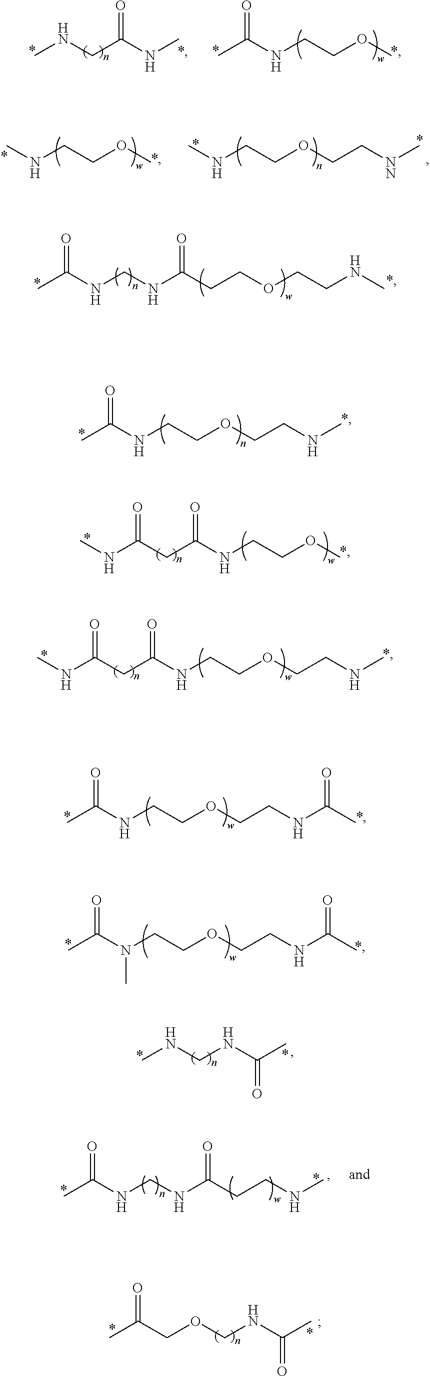

n and w are each individually integers from 1 to 9 and
* denotes the attachment to either T_{L} or S_{L}.

In one embodiment, L_{I} is according to

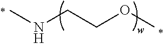

wherein w is an integer between 1 to 9, wherein * denotes the attachment to either T_{L} or S_{L}.

In one embodiment, L_{I} is according to

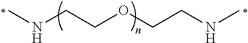

wherein n is an integer between 1 to 8, wherein * denotes the attachment to either T_{L} or S_{L}.

In one embodiment, L_{I} is according to

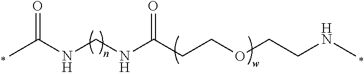

Wherein n and w are integers between 1 to 8, wherein * denotes the attachment to either T_{L} or S_{L}.

In one embodiment, L_{I} is according to

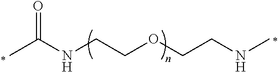

wherein n is an integer between 1 to 9, wherein * denotes the attachment to either T_{L} or S_{L}.

In one embodiment, L, is selected from one of the group consisting of formula (XVIa) to Formula (XVIae) in Table Z-II:

TABLE Z-II

| | |
|---|---|
| 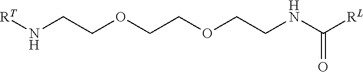 | XVIa |
| 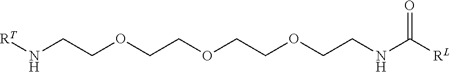 | XVIb |
| 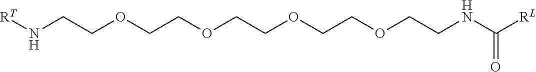 | XVIc |
| 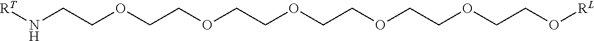 | XVId |
| 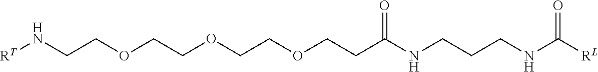 | XVIe |
| 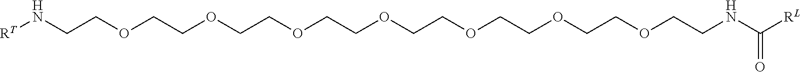 | XVIf |
| 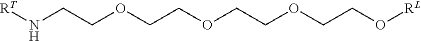 | XVIg |
| 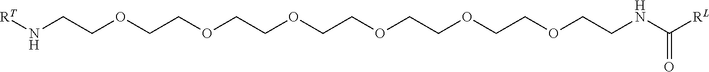 | XVIh |
| 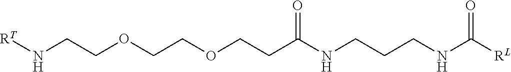 | XVIi |
| 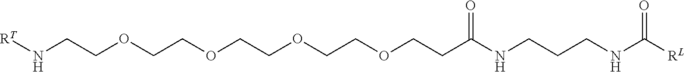 | XVIj |
| 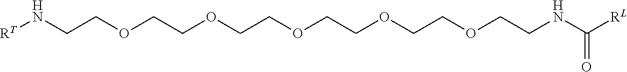 | XVIk |
| 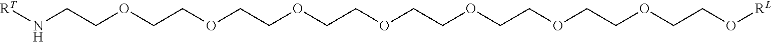 | XVIm |
| 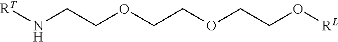 | XVIn |
| 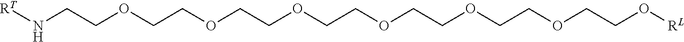 | XVIo |
| 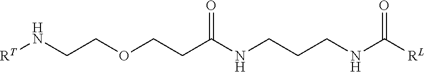 | XVIp |
| 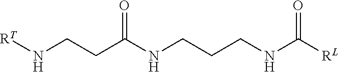 | XVIq |
| 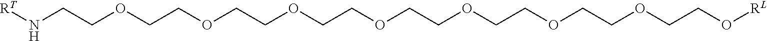 | XVIr |
| 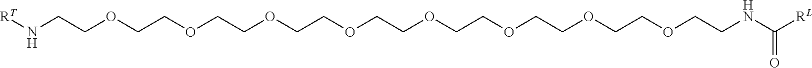 | XVIt |
| 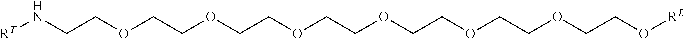 | XVIu |
| 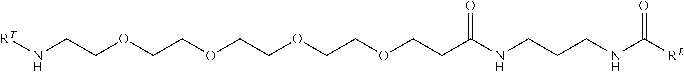 | XVIw |
| 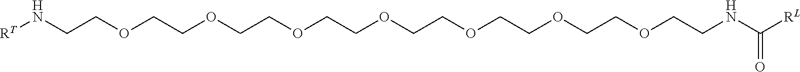 | XVIx |
| 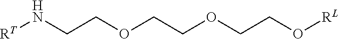 | XVIy |
| 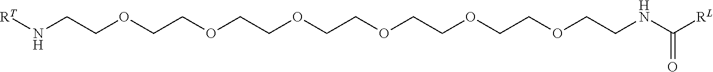 | XVIz |
| 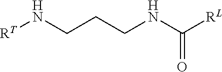 | XVIaa |
| 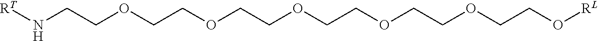 | XVIab |
| 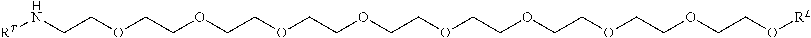 | XVIac |
| 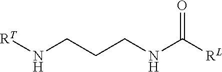 | XVIad |
| 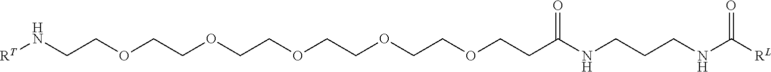 | XVIae |

wherein R^{L} denotes the point of attachment to S_{L} and R^{T} denotes the point of attachment to T_{L}.

In one embodiment, L, is selected from one of the group consisting of formulas XVIaf to XVIbw in Table Z-III:

TABLE Z-III

| | |
|---|---|
| 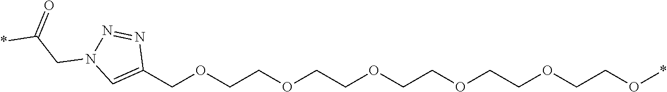 [SMILES: *OCCOCCOCCOCCOCCOCc1cn(CC(*)=O)nn1] | XVIaf |
| 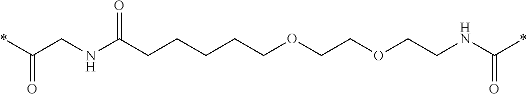 [SMILES: *C(=O)CNC(=O)CCCCCOCCOCCNC(*)=O] | XVIag |
| 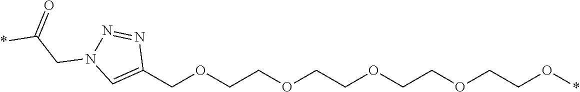 [SMILES: *OCCOCCOCCOCCOCc1cn(CC(*)=O)nn1] | XVIah |
| 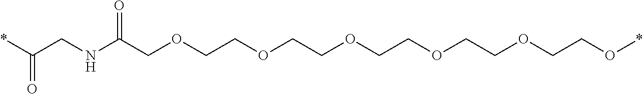 [SMILES: *OCCOCCOCCOCCOCCOCC(=O)NCC(*)=O] | XVIai |
| 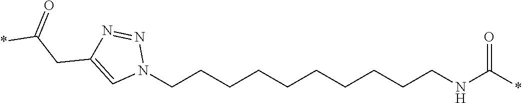 [SMILES: *C(=O)Cc1cn(CCCCCCCCCCNC(*)=O)nn1] | XVIaj |
| 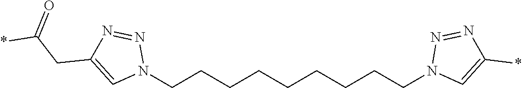 [SMILES: *C(=O)Cc1cn(CCCCCCCCCn2cc(*)nn2)nn1] | XVIak |
| 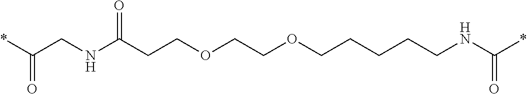 [SMILES: *C(=O)CNC(=O)CCOCCOCCCCCNC(*)=O] | XVIal |
| 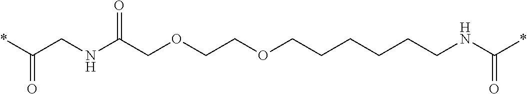 [SMILES: *C(=O)CNC(=O)COCCOCCCCCCNC(*)=O] | XVIam |
| 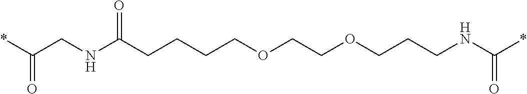 [SMILES: *C(=O)CNC(=O)CCCCOCCOCCCNC(*)=O] | XVIan |
| 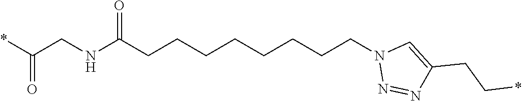 [SMILES: *CCc1cn(CCCCCCCCC(=O)NCC(*)=O)nn1] | XVIao |
| 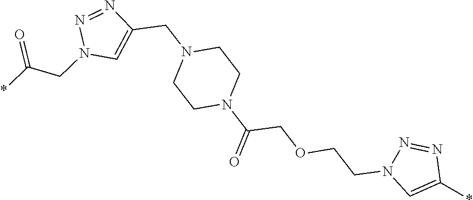 [SMILES: *C(=O)Cn1cc(CN2CCN(C(=O)COCCn3cc(*)nn3)CC2)nn1] | XVIap |
| 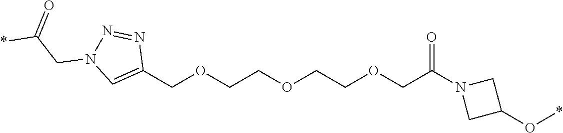 [SMILES: *OC1CN(C(=O)COCCOCCOCc2cn(CC(*)=O)nn2)C1] | XVIaq |
| 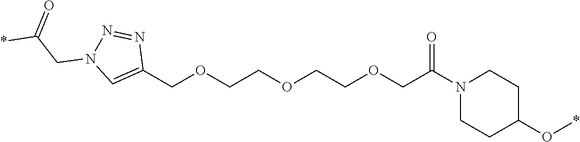 [SMILES: *OC1CCN(C(=O)COCCOCCOCc2cn(CC(*)=O)nn2)CC1] | XVIar |
| 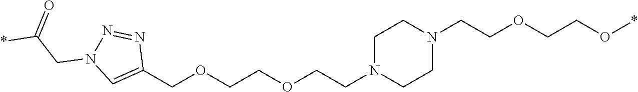 [SMILES: *OCCOCCN1CCN(CCOCCOCc2cn(CC(*)=O)nn2)CC1] | XVIas |
| 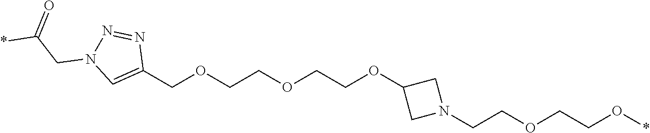 [SMILES: *OCCOCCN1CC(OCCOCCOCc2cn(CC(*)=O)nn2)C1] | XVIat |
| 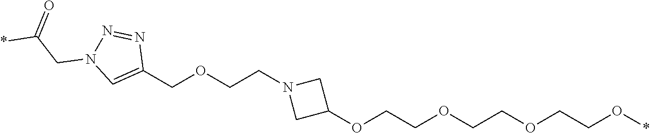 [SMILES: *OCCOCCOCCOC1CN(CCOCc2cn(CC(*)=O)nn2)C1] | XVIau |
| 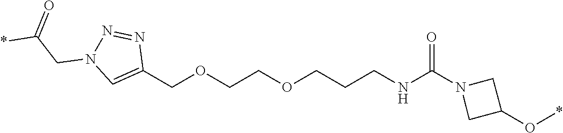 [SMILES: *OC1CN(C(=O)NCCCOCCOCc2cn(CC(*)=O)nn2)C1] | XVIaw |
| 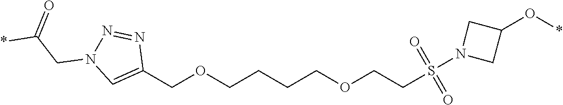 [SMILES: *OC1CN(S(=O)(=O)CCOCCCCOCc2cn(CC(*)=O)nn2)C1] | XVIax |
| 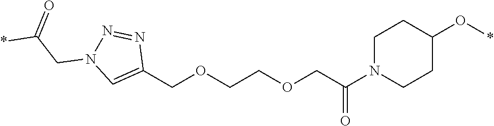 [SMILES: *OC1CCN(C(=O)COCCOCc2cn(CC(*)=O)nn2)CC1] | XVIay |
| 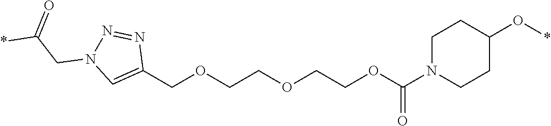 [SMILES: *OC1CCN(C(=O)OCCOCCOCc2cn(CC(*)=O)nn2)CC1] | XVIaz |
| 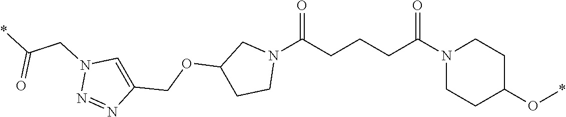 [SMILES: *OC1CCN(C(=O)CCCC(=O)N2CCC(OCc3cn(CC(*)=O)nn3)C2)CC1] | XVIba |
| 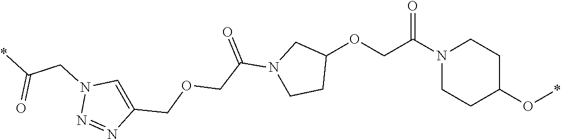 [SMILES: *OC1CCN(C(=O)COC2CCN(C(=O)COCc3cn(CC(*)=O)nn3)C2)CC1] | XVIbb |
| 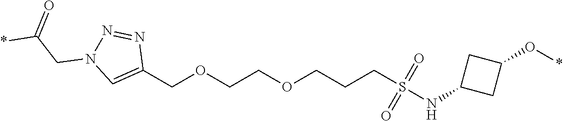 [SMILES: *O[C@H]1C[C@@H](NS(=O)(=O)CCCOCCOCc2cn(CC(*)=O)nn2)C1] | XVIbc |
| 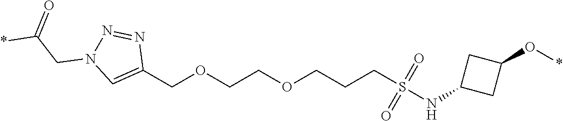 [SMILES: *O[C@H]1C[C@H](NS(=O)(=O)CCCOCCOCc2cn(CC(*)=O)nn2)C1] | XVIbd |
| 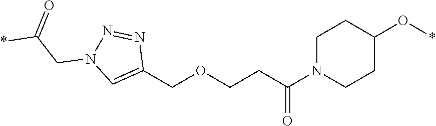 [SMILES: *OC1CCN(C(=O)CCOCc2cn(CC(*)=O)nn2)CC1] | XVIbe |
| 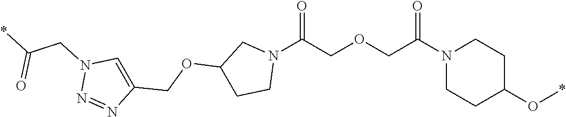 [SMILES: *OC1CCN(C(=O)COCC(=O)N2CCC(OCc3cn(CC(*)=O)nn3)C2)CC1] | XVIbf |
| 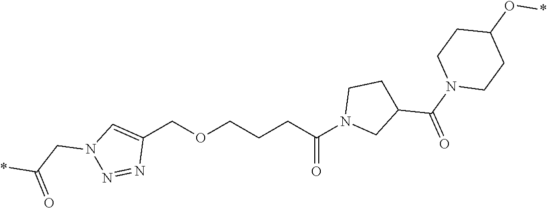 [SMILES: *OC1CCN(C(=O)C2CCN(C(=O)CCCOCc3cn(CC(*)=O)nn3)C2)CC1] | XVIbg |
| 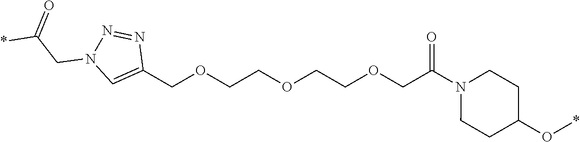 [SMILES: *OC1CCN(C(=O)COCCOCCOCc2cn(CC(*)=O)nn2)CC1] | XVIbh |
| 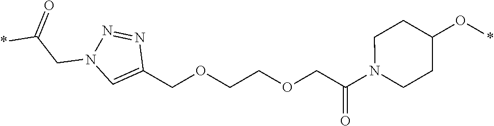 [SMILES: *OC1CCN(C(=O)COCCOCc2cn(CC(*)=O)nn2)CC1] | XVIbi |
| 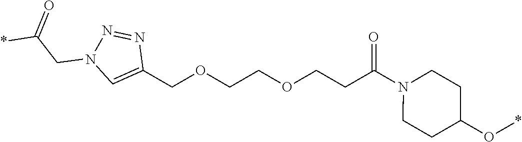 [SMILES: *OC1CCN(C(=O)CCOCCOCc2cn(CC(*)=O)nn2)CC1] | XVIbj |
| 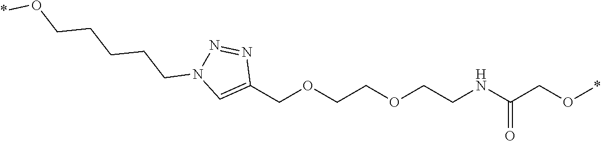 [SMILES: *OCCCCCn1cc(COCCOCCNC(=O)CO*)nn1] | XVIbk |
| 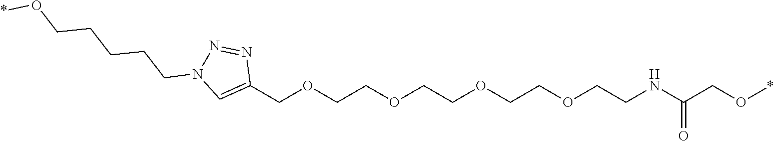 [SMILES: *OCCCCCn1cc(COCCOCCOCCOCCNC(=O)CO*)nn1] | XVIbl |
| 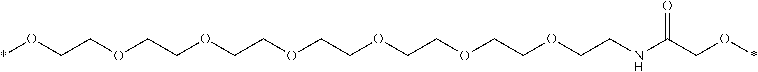 [SMILES: *OCCOCCOCCOCCOCCOCCOCCNC(=O)CO*] | XVIbm |
| 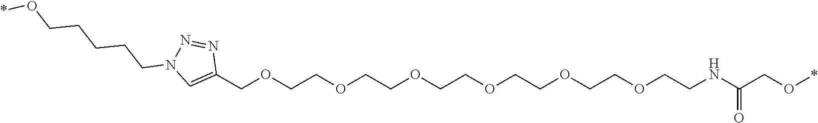 [SMILES: *OCCCCCn1cc(COCCOCCOCCOCCOCCOCCNC(=O)CO*)nn1] | XVIbn |
| 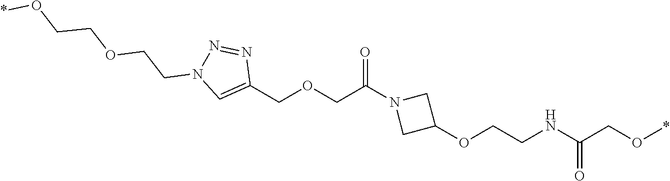 [SMILES: *OCCOCCn1cc(COCC(=O)N2CC(OCCNC(=O)CO*)C2)nn1] | XVIbo |
| 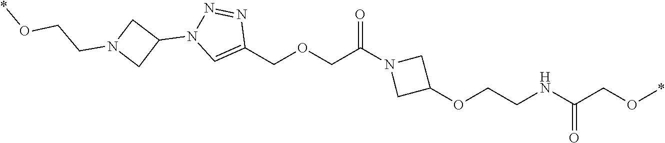 [SMILES: *OCCN1CC(n2cc(COCC(=O)N3CC(OCCNC(=O)CO*)C3)nn2)C1] | XVIbp |
| 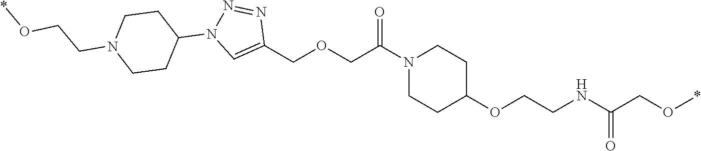 [SMILES: *OCCN1CCC(n2cc(COCC(=O)N3CCC(OCCNC(=O)CO*)CC3)nn2)CC1] | XVIbq |
| 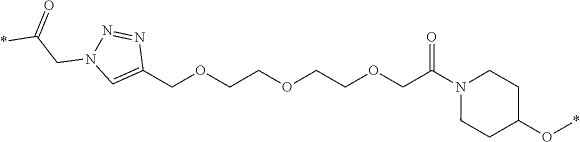 [SMILES: *OC1CCN(C(=O)COCCOCCOCc2cn(CC(*)=O)nn2)CC1] | XVIbr |
| 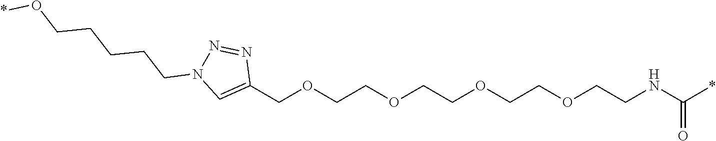 [SMILES: *OCCCCCn1cc(COCCOCCOCCOCCNC(*)=O)nn1] | XVIbs |
| 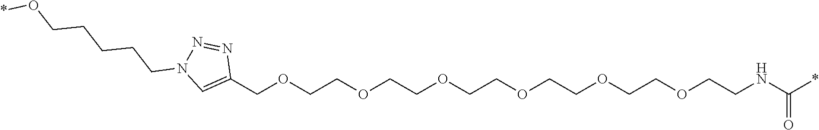 [SMILES: *OCCCCCn1cc(COCCOCCOCCOCCOCCOCCNC(*)=O)nn1] | XVIbt |
| 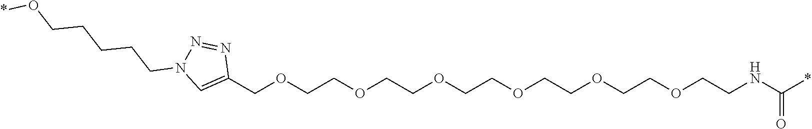 [SMILES: *OCCCCCn1cc(COCCOCCOCCOCCOCCOCCNC(*)=O)nn1] | XVIbu |
| 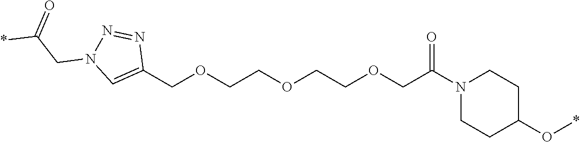 [SMILES: *OC1CCN(C(=O)COCCOCCOCc2cn(CC(*)=O)nn2)CC1] | XVIbw |

wherein * denotes the attachment either with S_{L} or T_{L}.

In one embodiment, the linker L_{I} is according to any one of formulas XVIaf to XVIbw in Table Z-III. The linker as shown in table Z-III, such as formulas XVIaf to XVIbw, may be connected to S_{L} and T_{L} at either position marked with *. For example, in one embodiment, the linkers shown in table Z-III are connected to T_{L} through the position marked with * that appears in the left of the formula shown in Table Z-III, and connected to S_{L} through the position marked with * in the right of the formula shown in Table Z-III.

In one embodiment the linker is a peptide. Thus, the linker may produce by methods to prepare peptides as known in the art, such as a peptide linker produce by solid-phase synthesis using protected amino acids or by expression of a suitable vector in an organism of choice.

In one embodiment, L_{I} is a peptide of a length between 1 to 30 amino acids, such as 3 to 20 amino acids.

In one embodiment, L, is a peptide of a length between 3 to 20 amino acids consisting of any combination of glycine, serine and cysteine.

In one embodiment, L_{I} is a peptide of a length between 3 to 20 amino acids consisting of any combination of glycine and serine.

In one embodiment, L_{I} is a peptide selected from the group consisting of:

(SEQ ID NO.: 131)
GGSGGGGSGGGGSGG
(SEQ ID NO.: 132)
GGSGGGG
(SEQ ID NO.: 133)
GGGGSGGGGSGGGGSGG
(SEQ ID NO.: 134)
GGSGGGGSGGGGS
(SEQ ID NO.: 135)
GGSGGGGSGGG
(SEQ ID NO.: 136)
GGSGGGGSG
(SEQ ID NO.: 137)
GGSGGGG
(SEQ ID NO.: 138)
GGSGG
(SEQ ID NO.: 139)
GGS
(SEQ ID NO.: 140)
CGGSGGGGSGGGGSGG
(SEQ ID NO.: 131)
In one embodiment, L_{I} is GGSGGGGSGGGGSGG.
(SEQ ID NO.: 132)
In one embodiment, L_{I} is GGSGGGG.
(SEQ ID NO.: 133)
In one embodiment, L_{I} is GGGGSGGGGSGGGGSGG.
(SEQ ID NO.: 134)
In one embodiment, L_{I} is GGSGGGGSGGGGS.
(SEQ ID NO.: 135)
In one embodiment, L_{I} is GGSGGGGSGGG.
(SEQ ID NO.: 136)
In one embodiment, L_{I} is GGSGGGGSG.
(SEQ ID NO.: 137)
In one embodiment, L_{I} is GGSGGGG.
(SEQ ID NO.: 138)
In one embodiment, L_{I} is GGSGG.
(SEQ ID NO.: 139)
In one embodiment, L_{I} is GGS.
(SEQ ID NO.: 140)
In one embodiment, L_{I} is CGGSGGGGSGGGGSGG

In one embodiment, L_{I} is connected to S_{L} via the C-terminus of L_{I} and L_{I} is connected to T_{L} via the N-terminus of L_{I}.

### Bifunctional Compounds:

### Compounds

In one embodiment, the bifunctional compound is selected from any one of compounds BF030 to BF149 as shown in Table A in the section “List of Compounds” or a pharmaceutically acceptable salt thereof.

In one embodiment, the bifunctional compound according to the present disclosure has:
a) S_{L} selected from any one from the group consisting of formulas any one of formulas IIIi, IIIj, IIIk, IIIm, IIIn, and IIIo; or selected from formula D-I as described in the section “Binding of Sortilin” above; and
b) is able to bind the target molecule with a dissociation constant (K_{D}) of less than 50 μM, such as less than 40 μM, such as less than 30 μM, such as less than 20 μM, such as less than 10 μM, such as less than 5 μM, such as less than 4 μM, such as less than 3 μM, such as less than 2 μM, such as less than 1 μM, such as less than 0.8 μM, such as less than 0.6 μM, such as less than 0.5 μM, such as less than 0.4 μM, such as less than 0.3 μM, such as less than 0.2 μM, such as less than 0.1 μM.

In one embodiment, the bifunctional compound according to the present disclosure has:
a) S_{L} selected from any one from the group consisting of formulas any one of formulas IIIi, IIIj, IIIk, IIIm, IIIn, and IIIo; or selected from formula D-I as described in the section “Binding of Sortilin” above; and
b) L, selected from any one of formulas any one of formulas XVIaf to XVIbw in Table Z-III in the section “Linkers” above; and
c) T_{L} according to any one of formulas XVIIa-3, XVIIa-4,XVIIc-1 and XVIIc-2 as described in the section “Targeting warhead” above.

In one embodiment, the bifunctional compound according to the present disclosure is selected from any one of compounds BF001 to BF028 as shown in Table A in the section “List of Compounds”.

### Peptides

In one embodiment, the bifunctional compound is selected from any one of the group consisting of:

(SEQ ID NO: 141)
Biotin-GGSGGGGSGRQLL-OH
(SEQ ID NO.: 142)
Biotin-GGSGGGGSGGGGSRQLL-OH
(SEQ ID NO.: 143)
Biotin-GGSGGGGFQLL-OH
(SEQ ID NO.: 144)
Biotin-GGSGGFQLL-OH
(SEQ ID NO.: 145)
Biotin-GGSGGGGSGFQLL-OH
(SEQ ID NO.: 146)
Biotin-GGSGGGGSGGGGSFQLL-OH
(SEQ ID NO.: 147)
Biotin-GGSGGGGSGGGGSGGRQLL-OH
(SEQ ID NO.: 148)
Biotin-GGSGGGGSGGGFQLL-OH
(SEQ ID NO.: 149)
Biotin-GGSGGGGSGGGRQLL-OH
(SEQ ID NO.: 150)
Biotin-GGSGGGGRQLL-OH
(SEQ ID NO.: 151)
Biotin-GGSGGRQLL-OH
(SEQ ID NO.: 152)
Biotin-GGSRQLL-OH
(SEQ ID NO.: 153)
Biotin-GGRQLL-OH
(SEQ ID NO.: 154)
Biotin-RQLL-OH
((SEQ ID NO.: 155)
Biotin-GGSGGGGSGGGGSGGFQLL-OH
(SEQ ID NO.: 188)
Biotin-REAPRWDAPLRDPALFQLL-NH2
(SEQ ID NO.: 189)
Biotin-REAPRWDAPLRDPALFQLL-OH
(SEQ ID NO.: 190)
Biotin-REAPRWDAPLRDPALRQLL-NH2,
and
(SEQ ID NO.: 191)
Biotin-REAPRWDAPLRDPALRQLL-OH.

### Protein

In one embodiment, the bifunctional compound is:

(SEQ ID NO.: 158)
MGGTHHHHHHENLYFQGQVQLQESGGGLVQPGGSLRLSCAASGRTISR
YAMSWFQAPGKEREFVAVARRSGDGAFYADSVQGRFTVSRDDAKNTVY
LQMNSLKPEDTAVYCAIDSDTFYSGSYDYWGQGTQVTVSSEGGGGSGG
GGSGGGGSGGRQLL

In one embodiment, the bifunctional compound has T_{L} consisting of the monoclonal monoclonal antibody alirocumab conjugated to at least 1 unit-L_{I}-S_{L} consisting of: CGGSGGGGSGGGGSGGRQLL (SEQ ID NO.: 175); wherein SEQ ID NO.: 175 is conjugated via its N-terminus. This conjugate of alirocumab is herein described as Alirocumab-CGGSGGGGSGGGGSGGRQLL (SEQ ID NO.: 159) or Alirocumab-link-RQLL or Alircoumab-linker-RQLL.

A variant conjugate without the sequence RQLL is also disclosed, and is herein referred to as:
Alirocumab-CGGSGGGGSGGGGSGG (SEQ ID NO.: 160) or Alirocumab-link or Alirocumab-linker.

The product described in SEQ ID NO.: 159 may have more than one unit of (SEQ ID NO.: 175) conjugated to the antibody, such as 2 units or more units, such as 3 or more units, such as 4 or more units.

In one embodiment, the bifunctional compound has T_{L} consisting of the monoclonal monoclonal antibody adalimumab conjugated to at least 1 unit-L_{I}-S_{L} consisting of: CGGSGGGGSGGGGSGGRQLL (SEQ ID NO.: 175), wherein SEQ ID NO.: 175 is conjugated via its N-terminus. This conjugate of adalimumab is herein referred to as:
Adalimumab-CGGSGGGGSGGGGSGGRQLL (SEQ ID NO.: 161) or
Adalimumab-link-RQLL or Adalimumab-linker-RQLL.

In one embodiment, the bifunctional compound has T_{L} consisting of the monoclonal monoclonal antibody adalimumab conjugated to at least 1 unit-L_{I}-S_{L} consisting of the group consisting of:

(SEQ ID NO.: 175)
CGGSGGGGSGGGGSGGRQLL,
(SEQ ID NO.: 184)
CREAPRWDAPLRDPALRQLL
(SEQ ID NO.: 185)
CREAPRWDAPLRDPALRQLL-NH2
(SEQ ID NO: 186)
CREAPRWDAPLRDPALFQLL-OH
and
(SEQ ID NO: 187)
CREAPRWDAPLRDPALFQLL-NH2

wherein SEQ ID NO.: 175 or SEQ ID NO.: 184 to 187 are conjugated via its N-terminus. These conjugates of adalimumab are herein referred to as:

(SEQ ID NO.: 161)
Adalimumab-CGGSGGGGGGGGSGGRQLL
(SEQ ID NO.: 192)
Adalimumab-CREAPRWDAPLRDPALRQLL
(SEQ ID NO.: 193)
Adalimumab-CREAPRWDAPLRDPALRQLL-NH2
(SEQ ID NO: 194)
Adalimumab-CREAPRWDAPLRDPALFQLL-OH
(SEQ ID NO.: 195)
Adalimumab-CREAPRWDAPLRDPALFQLL-NH2.

A variant conjugate without the sequence RQLL is also disclosed, and is herein referred to as:
Adalimumab-CGGSGGGGSGGGGSGG (SEQ ID NO.: 162) or Adalimumab-link or Adalimumab-linker.

The product described in SEQ ID NO.: 161 may have more than one unit of (SEQ ID NO.: 175) conjugated to the antibody, such as 2 units or more units, such as 3 or more units, such as 4 or more units. Similarly the products described as conjugates by SEQ ID NO.: 192 to 195 may have more than one unit of SEQ ID NO.: 184 to SEQ ID NO.: 187, such as 2 units or more units, such as 3 or more units, such as 4 or more units.

The number of -L_{I}-S_{L} conjugated per antibody may be expressed as an average number per antibody: Nₐ. In one embodiment, Nₐ is an integer or a decimal between 1 and 10. In one embodiment, the product described in sEQ ID NO.: 161 may have a number of -L_{I}-S_{L} units of SEQ ID NO.: 175 such that Nₐ is between 1 and 10. In one embodiment, the products described in any one of SEQ ID NO.: 192 to 195, may have a number of -L_{I}-S_{L} units of SEQ ID NO.: 184 to 187 such that Nₐ is between 1 and 10.

The bifunctional compounds according to the present invention are able to bind sortilin through the sortilin binding moiety (S_{L}) and a target protein through the protein targeting moiety (T_{L}).

Thus in one embodiment, the bifunctional compounds of the present invention are able to bind to sortilin and to the target protein at the same time. This means, that the bifunctional compounds according to the present disclosure form a ternary complexes with sortilin and the target protein. In a ternary complex, sortilin and the target protein are bound simultaneously to the bifunctional compound.

The formation of ternary complexes can be measured in different ways as known in the art. For example, ternary complex formation can be measure through Förster's resonance energy transfer (FRET) assays or through timer-resolved Förster's resonance energy transfer (TR-FRET) assays. As measured by these assays an increase in the homogeneous time-resolved fluorescence (HTRF) ratio indicates formation of ternary complexes.

As demonstrated in the examples, bifunctional compounds according to the present disclosure are able to bind both sortilin and target proteins, and form ternary complexes.

In one embodiment, the bifunctional compound according to the present disclosure is able to bind to sortilin at the cell surface. In another embodiment, the bifunctional compound is able to form a ternary complex with sortilin and the target molecule at the cell surface.

In one embodiment, upon binding of S_{L} to sortilin located on the cell surface and binding of T_{L} to the target protein, the target protein is internalized into said cell. In one embodiment, the target protein is degraded after internalization into said cell. In one embodiment, the degradation of the target protein happens in the lysosomal compartments.

In one embodiment, the bifunctional compound according to the present disclosure has a dissociation constant of binding to sortilin of less than 50 μM, such as less than 40 μM, such as less than 30 μM, such as less than 20 μM, such as less than 10 μM, such as less than 5 μM, such as less than 4 μM, such as less than 3 μM, such as less than 2 μM, such as less than 1 μM, such as less than 0.8 μM, such as less than 0.6 μM, such as less than 0.5 μM, such as less than 0.4 μM, such as less than 0.3 μM, such as less than 0.2 M, such as less than 0.1 μM, such as less than 0.05 μM, such as less than 0.01 μM and a dissociation constant of binding to the target protein of less than 50 μM, such as less than 40 μM, such as less than 30 μM, such as less than 20 μM, such as less than 10 μM, such as less than 5 μM, such as less than 4 μM, such as less than 3 μM, such as less than 2 μM, such as less than 1 M, such as less than 0.8 μM, such as less than 0.6 μM, such as less than 0.5 μM, such as less than 0.4 μM, such as less than 0.3 μM, such as less than 0.2 μM, such as less than 0.1 μM.

In one embodiment, the target protein is TNFa. Thus, in one embodiment, the bifunctional compound according to the present disclosure is able to form a ternary complex between sortilin and TNFa. In one embodiment, the bifunctional compound is able to bind to sortilin and TNFa at the same time.

In one embodiment, the bifunctional compound according to the present disclosure has a dissociation constant of the binding of S_{L} to sortilin is of less than 50 μM, such as less than 2 μM, such as less than 0.5 μM, preferably less than 0.1 μM and the dissociation constant of the binding of T_{L} to TNFa is of less than 100 μM, such as less than 0.5 μM, such as less than 0.1 μM.

In one embodiment, the bifunctional compound according to the present disclosure has a dissociation constant of the binding to sortilin is of less than 50 μM, such as less than 2 μM, such as less than 0.5 μM, preferably less than 0.1 μM and the dissociation constant of to TNFa is of less than 100 μM, such as less than 0.5 μM, such as less than 0.1 μM.

In one embodiment, the bifunctional compound provides that upon binding of S_{L} to sortilin located on the cell surface and binding of T_{L} TNFa, TNFa is internalized into said cell. To assess that the target proteins are internalized, methods like detection of the target protein in the supernatant of cell cultures may be used. Any suitable methods such as ELISA, detection of fluorescence of conjugates, HPLC, gel electrophoresis combined with staining, like SDS-PAGE or western blotting, or other well known techniques in the art may be used. The presence of the target protein or its fragments inside cells may also be assessed with similar methods, after lysis of the cells.

The example demonstrate that bifunctional compounds according to the present disclosure are able to promote internalization of target proteins into cells through sortilin mediated binding. The examples also demonstrate that the internalization leads to degradation of the target proteins.

In one embodiment, TNFa is degraded after internalization into the cell.

### Preparation of Bifunctional Compounds:

The following abbreviations refer respectively to the definitions below:
Ac (acetyl), aq (aqueous), h (hour), g (gram), L (liter), mg (milligram), MHz (Megahertz), μM (micromolar), min (minute), mm (millimeter), mmol (millimole), mM (millimolar), m.p. (melting point), equiv (equivalent), mL (milliliter), μL (microliter), ACN (acetonitrile), AcOH (acetic acid), BINAP (2,2′-bis(disphenylphosphino)-1,1′-binaphthalene, BOC (tert-butoxy-carbonyl), CBZ (carbobenzoxy), CDCl3 (deuterated chloroform), CD3OD (deuterated methanol), CH₃CN (acetonitrile), c-hex (cyclohexane), DCC (dicyclohexyl carbodiimide), DCM (dichloromethane), DHP (O-(2,4-dinitrophenyl)-hydroxylamine), dppf (1,1′-bis(diphenylphosphino) ferrocene), DIC (diisopropyl carbodiimide), DIEA (diisopropylethyl-amine), DMF (dimethylformamide), DMSO (dimethylsulfoxide), DMSO-d6 (deuterated dimethylsulfoxide), EDC (1-(3-dimethyl-amino-propyl)-3-ethylcarbodiimide), ESI (Electro-spray ionization), EtOAc (Ethyl acetate), Et2O (diethyl ether), EtOH (ethanol), FMOC (fluorenylmethyloxycarbonyl), HATU (dimethylamino-([1,2,3]triazolo[4,5-b]pyridin-3-yloxy)-methylene]-dimethyl-ammonium hexafluorophosphate), HPLC (High Performance Liquid Chromatography), i-PrOH (2-propanol), K2CO3 (potassium carbonate), LC (Liquid Chromatography), m-CPBA (3-chloroperbenzoic acid), MD Autoprep (Mass directed Autoprep), MeOH (methanol), MgSO4 (magnesium sulfate), MS (mass spectrometry), MSH (O-mesitylenesulfonylhydroxylamine), MTBE (Methyl tert-butyl ether), Mtr. (4-Methoxy-2, 3, 6-trimethylbenzensulfonyl), MW (microwave), NBS (N-bromo succinimide), NaHCO₃ (sodium bicarbonate), NaBH4 (sodium borohydride), NMM (N-methyl morpholine), NMR (Nuclear Magnetic Resonance), POA (phenoxyacetate), Py (pyridine), PyBOP®(benzotriazole-1-yl-oxy-tris-pyrrolidino-phosphonium hexafluorophosphate), RT (room temperature), Rt (retention time), SFC (supercritical fluid chromatography), SPE (solid phase extraction), T3P (propylphosphonic anhydride), TBAF (tetra-n-butylammonium fluoride), TBTU (2-(1-H-benzotriazole-1-yl)-1,1,3,3-tetramethyluromium tetrafluoro borate), TEA (triethylamine), TFA (trifluoroacetic acid), THF (tetrahydrofurane), TLC (Thin Layer Chromatography), UV (Ultraviolet).

In general, the compounds according to Formula (I) and related formulae of this invention may be prepared from readily available starting materials. If such starting materials are not commercially available, they may be prepared by standard synthetic techniques. In general, the synthesis pathways for any individual compound of Formula (I) and related formulae will depend on the specific substituents of each molecule, such factors being appreciated by those having ordinary skill in the art. The following general methods and procedures described hereinafter in the examples may be employed to prepare compounds of Formula (I) and related formulae. Reaction conditions depicted in the following schemes, such as temperatures, solvents, or co-reagents, are given as examples only and are not restrictive. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by a person skilled in the art, using routine optimisation procedures. For all the protection and deprotection methods, see Philip J. Kocienski, in “Protecting Groups”, Georg Thieme Verlag Stuttgart, New York, 1994 and, Theodora W. Greene and Peter G. M. Wuts in “Protective Groups in Organic Synthesis”, Wiley Interscience, 3rd Edition 1999.

Depending on the nature of T_{L}, L_{I} and S_{L}, different synthetic strategies may be selected for the synthesis of compounds of Formula (I). In the process illustrated in the following schemes, T_{L}, L_{I} and S_{L} are as above-defined in the description unless otherwise mentioned.

Compounds of Formula (I), wherein T_{L}, L_{I} and S_{L} are defined as above, can be prepared from alternative compounds of Formula (I), using suitable interconversion procedures such as those described hereinafter in the examples, or conventional interconversion procedures well known by one skilled in the art.

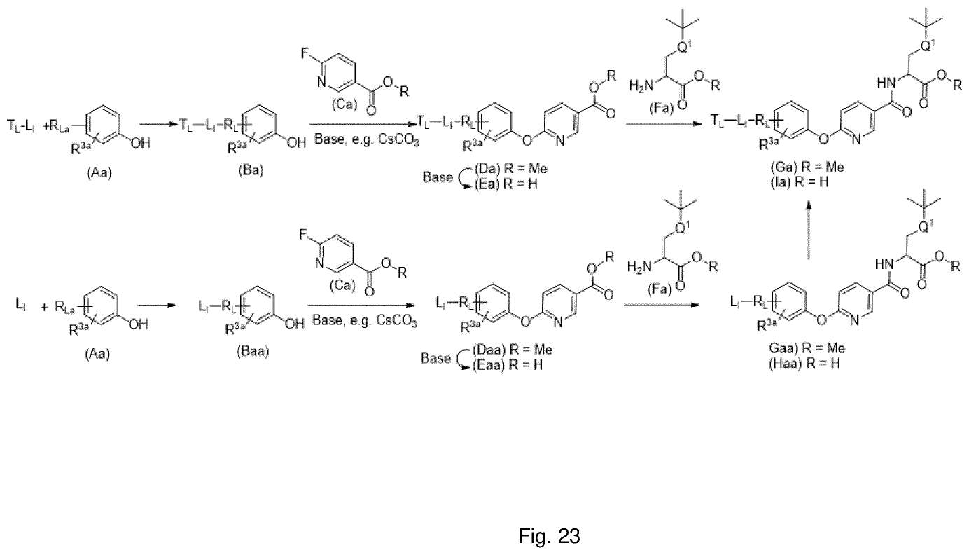

In FIG. 23, R denotes H or methyl and Q¹ denotes —CH₂— or a bond.

PG is a suitable protecting group, which is compatible with the chemistry described in Scheme 1 to 19. Preferred groups PG are the following: Carbobenzyloxy (Cbz), p-Methoxybenzyl carbonyl (Moz or MeOZ) group, tert-Butyloxycarbonyl (BOC) group, 9-Fluorenylmethyloxycarbonyl (FMOC) group, Alkanoyl group, such as the Acetyl (Ac) group, Benzoyl (Bz) group, Benzyl (Bn) group, Carbamate group, p-Methoxybenzyl (PMB), 4-Dimethoxybenzyl (DMPM), p-methoxyphenyl (PMP) group, Arylsulfonyl group such as the Tosyl (Ts) or benzolsulfonyl group.

Compounds of formula (I), wherein TL, LI and SL are defined as above, can be prepared from the reaction of compound composed with TL-LI moieties with SL or from the reaction of compound composed with LI-SL moieties with TL using methods and reactions known by a person skilled in the art. Such reactions can be but are not limited to amide bond formation, aromatic substitution, alkylation and metal catalysed cross-coupling reaction, using conditions known by a person skilled in the art and as described below in the examples (Scheme 1).

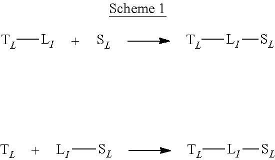

Alternatively, precursors of T_{L}, L_{I} or S_{L} can be used for the synthesis of compound of formula (I), by reacting with the other moieties composing compound of formula (I).

Further steps would yield compound of formula (I), using methods and reactions known by a person skilled in the art.

As example, but not limited to this case, when S_{L} corresponds to (IIIa), T_{L}-L_{I} or L_{I} moieties can react with phenol derivative (Aa), yielding intermediates (Ba) or (Baa) respectively, wherein R_{La} corresponds to the functional group reacting with L_{I} moiety yielding R_{L} as attachment with L_{I} (FIG. 23). The reaction of intermediate (Ba) or (Baa) with ester (Ca) via aromatic substitution would afford ester (Da) or (Daa) respectively. After saponification conditions, acids (Ea) or (Eaa) would be obtained respectively.

Alternatively, aromatic substitution with the corresponding acid (Ca, R═H), would yield directly acids (Ea) and (Eaa) respectively. Amide coupling with ester (Fa) would provide intermediates (Ga) and (Gaa) respectively, which after saponification would result into compound of formula (Ia) or intermediate (Haa). Further reaction of (Haa) with T_{L} moiety under conditions described in the examples below or known by the person skilled in the art would yield compound of formula (Ia).

### Medical Use:

The present invention focuses on the degradation of circulating extracellular proteins that mediate diseases, for example, involving immunity, inflammation, hematopoiesis/blood disorders (including those caused or exacerbated by blood vessel formation) and abnormal cellular proliferation such as tumors and cancer.

The bifunctional compounds of the present invention can he administered in any manner that allows the bifunctional compound to bind to the Extracellular Protein, typically in the blood stream, and carry it to the sortilin bearing cells for endocytosis and degradation. As such, examples of methods to deliver the degraders of the present invention include, but are not limited to, oral, intravenous, buccal, sublingual, subcutaneous and transnasal.

In one aspect, the invention provides bifunctional compounds for the use in the targeted sortilin-mediated lysosomal degradation of extracellular target molecules. In one embodiment, the target molecule is a disease or disorder associated protein.

In another aspect, the present disclosure relates to the use of bifunctional compounds as described herein for use as a medicament. The present disclosure is directed to pharmaceutical compositions comprising a bifunctional compound as described herein.

In yet another aspect, the present disclosure relates to a method of targeted lysosomal degradation of an extracellular target protein, comprising administering a bifunctional compound as described herein to a subject in need thereof.

In another aspect, the present disclosure relates to a method to reduce the plasma levels of a target molecule, comprising administering a bifunctional compound as described herein to a subject in need thereof.

In one aspect, the present disclosure provides bifunctional compounds as described herein for use in the treatment of a disorder or condition mediated by an extracellular protein in a subject in need thereof.

In one aspect, the present disclosure relates bifunctional compounds as described herein for use in the removal of an extracellular target molecule from the plasma of a subject.

In one embodiment, the disorder or disease is linked to abnormal levels of the extracellular protein. In one embodiment, the disorder or disease is linked to abnormally high levels of the extracellular target protein.

In one embodiment, the disorder or disease is linked to a mutated or misfolded extracellular protein.

In one embodiment, the extracellular target protein is selected from the group consisting of: TNF-α, PCSK9, ANGPTL-3, an antibody light chain, IgG, IgE, IgA IL-1, IL-2, IL-6, IFN-γ, VEGF, TFG-β1, IL-21, IL-22, IL-5, IL-10, IL-8, cholinestearase, human CCL2, carboxypeptidase B-2, neutrophil elastase, Factor Xa, Factor XI, Factor XIa, Factor XII, Factor XIII, prothrombin, coagulation factor VII, coagulation factor IX, fibroblast growth factor 1, FGF-2, fibronectin 1, kallikrein-1, lipoprotein lipase, human matrix metallopeptidase 1, macrophage migration inhibitory factor, transformin growth factor-p (TGF-p), thrombospondin-1 (TSP-T), CD40 ligand, urokinase-type plasminogen activator, plasminogen activator tissue type (TPA), Plasminogen (PLG), Plasminogen Activator Inhibitor-1, Placenta Growth Factor, Phospholipase A2 Group IB, Phospholipase A2 Group IIA, Complement factor B, Complement factor D, complement factor H, Complement Component 5 and complement C₁s.

In one embodiment, the extracellular protein is PCSK9. In a further embodiment, the extracellular target protein is PCSK9 and the disorder or condition is a disorder of lipoprotein metabolism. In a further embodiment, the disorder of lipoprotein metabolism is linked to abnormal PCSK9 levels. In another embodiment, the disorder is selected from dyslipidemia, hypercholesterolemia and coronary heart disease.

In one embodiment, the extracellular protein is TNF-x. In a further embodiment, the extracellular target protein is TNF-x and the disorder or condition is selected from the group consisting of rheumatoid arthritis, inflammatory bowel disease, graft-vs-host disease, ankylosing spondylitis, psoriasis, hidradenitis suppurativa, refractory asthma, systemic lupis erthyematosus, diabetes, and the induction of cachexia. In a further embodiment, the disorder is linked to abnormal TNF-α levels. In one embodiment, the disorder or condition is an inflammatory disease. In one embodiment, the disorder or condition is an autoimmune disease. In one embodiment, the disorder or condition is a cancer.

Thus, in one aspect the present disclosure provides for a method of targeted lysosomal degradation of TNFa, comprising administering an effective amount of the bifunctional compound as described herein.

In another aspect, the present disclosure provides for a method of removal of TNFa from the plasma of a patient or subject in need thereof, comprising administering a bifunctional compound as described herein.

In another aspect, the present disclosure provides for the use of a bifunctional compound according as described herein for the manufacture of a medicament for the treatment of a disease or condition.

In another aspect, the present disclosure provides for the use of a bifunctional compound according as described herein for the manufacture of a medicament for the treatment of a disorder or condition mediated by TNFa.

In one aspect, the present disclosure provides for a method of treatment of a disease or condition comprising administering a bifunctional compound as described herein to a subject in need thereof.

In one aspect, the present disclosure provides for a method of treatment of a disorder or condition mediated by TNFa, comprising administering a bifunctional compound according to the present disclosure to a subject in need thereof.

The examples demonstrate that bifunctional compounds according to the present disclosure that target TNFa promote removal of TNFa from the plasma of animals.

In one embodiment, the one embodiment, the extracellular protein is an antibody light chain or an IgG. In a further embodiment, the extracellular target protein is IgG and the disorder or condition is selected from the group consisting of type 1 autoimmune pancreatitis, interstitial nephritis, Riedel's thyroiditis, storiform fibrosis, Mikulicz's disease, Kuttner's tumor, inflammatory' pseudotumors, mediastinal fibrosis, retroperitoneal fibrosis (Ormond's disease), aortitis, periaortitis, proximal biliary strictures, idiopathic hypocomplementic tubulointerstitial nephritis, multifocal fibrosclerosis, pachymeningitis, pancreatic enlargement, tumefactive lesions, pericarditis, rheumatoid arthritis (RA), inflammatory bowel disease, multiple sclerosis, myasthenic gravis, thyroid eye disease, chronic inflammatory demyelinating polyneuropathy, warm autoimmune hemolytic anemia, ankylosing spondylitis, primary Sjogren's syndrome, psoriatic arthritis, and systemic lupus erythematosus (SLE), sclerosing cholangitis, and IgG monoclonal gammopathy, monoclonal gammopathy of undetermined significance (MGUS).

In one embodiment, the subject is a mammal. In one embodiment, the mammal is a human.

### EXAMPLES

### Example 1: Bifunctional Compound with Peptide Derived S_{L} Interact Directly with the Ectodomain of Sortilin

### Aim

In this example the equilibrium dissociation constant (K_{D}) describing the binary binding event between bifunctional compounds wherein S_{L} is derived from a peptide and the soluble ectodomain of sortilin-6his is measured by microscale thermophoresis (MST).

### Materials and Method

In these experiments a 12 point-titration series of peptide solution was prepared in TTP LVDS 384-well plates (SPT Labtech) in 1.2 uL total volume using a Mosquito pipetting robot (SPT Labtech) in a buffer composed of 16.7% DMSO (v/v) and 0.05% Tween20 (v/v). The plate was spun down before addition of 8.8 uL solution containing 114 nM sortilin-6his and 28.4 nM RED-tris-NTA (Nanotemper) in 57 mM Bis-Tris Propane pH 9, 57 mM NaCl, 0.05% Tween20 (v/v) to each well. At this point the plate was sealed with adhesive strip and placed on an Eppendorf thermomixer to shake at 600 rpm for 3 min before spinning on a Fisherbrand plate-spinner for 15 seconds and further incubation 1 hrs at 19° C. MST was measured on a Monolith NT. Automated (Nanotemper) in standard-treated capillaries at 5% LED-power and high MST-power. Dose-response was extracted from the raw thermographs at 20 sec hot-time and the dissociation constant was estimated by sigmoidal curve fitting in MO. Affinity analysis program (Nanotemper). All experiments were run in duplicates.

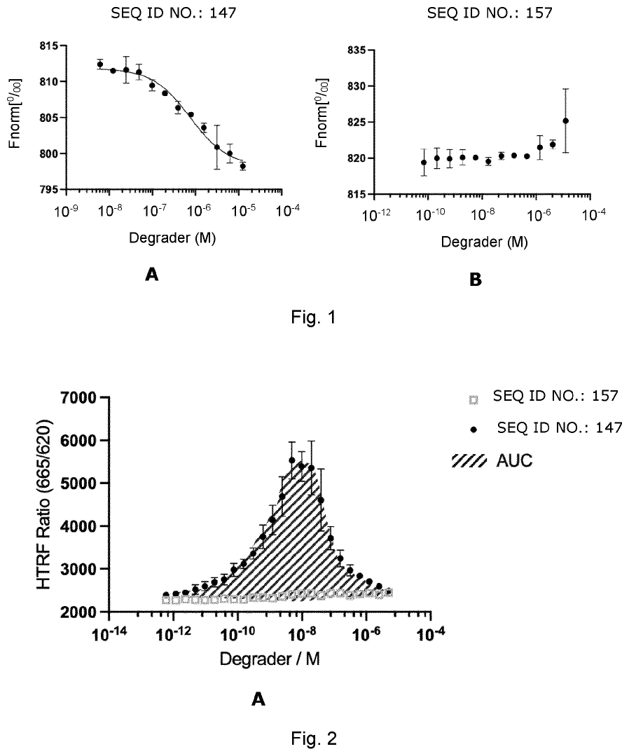

FIG. 1A shows an MST binding experiment using fluorescent sortilin-6his (100 nM) and compounds according to SEQ ID NO.: 147 resulting in a measured dissociation constant (K_{D}) of 703 nM (N=2). FIG. 1B shows the identical experimental setup as FIG A1 but titrating a compound according to SEQ ID NO.: 157 incapable of binding to fluorescent sortilin-6his due to specific amidation of the C-terminal.

### Conclusion

Compounds of the invention can bind sortilin.

TABLE 1
Binding to sortilin (Kd) (K_{D} < 100 nM: ++; K_{D} 100-2000 nM: +)

| SEQ ID NO.: | K_{D} score |
|---|---|
| 141 | ++ |
| 142 | ++ |
| 143 | ++ |
| 144 | ++ |
| 145 | ++ |
| 146 | + |
| 147 | + |
| 148 | + |
| 149 | + |
| 150 | ++ |
| 152 | + |
| 153 | ++ |
| 154 | ++ |
| 155 | + |
| 156 | no binding |
| 157 | no binding |

### Example 2: Bifunctional Compound with Peptide Derived Sy Facilitate Ternary Complex Formation Between Sortilin and a Target Protein

### Aim

Addressed in this example is the ability of bifunctional compounds compound wherein S_{L} is derived from a peptide to induce ternary complex formation composed of streptavidin, bifunctional compound and sortilin-6his. In this example, bifunctional molecules are composed at one end of a biotin molecule followed by peptide linkers of various length and composition and finally of a sortilin binding peptide sequence. Induction of ternary complex is measured by the proximity-based TR-FRET assay.

### Materials and Methods

In these experiments, the compounds of interest were titrated over 24-point in a 384-well TTP LVDS plate (SPT Labtech) to a final buffer composition of 50 mM Bis-Tris Propane pH 8, 50 mM NaCl, 0.05% Tween20, with or without 10% DMSO using a Mosquito LV pipetting robot (SPT Labtech).

This titration series (4 uL) was transferred to a black 384-well plate (Corning) and the following were added: 4 μL 10 nM MAb Anti-6HIS Tb (Cisbio) in 50 mM Bis-Tris Propane pH 8, 50 mM NaCl, 0.05% Tween20, 4 μL 250 nM Streptavidin-d2 (Cisbio) in 50 mM Bis-Tris Propane pH 8, 50 mM NaCl, 0.05% Tween20, 4 μL 200 nM Sortilin-6His in 50 mM Bis-Tris Propane pH 8, 50 mM NaCl, 0.05% Tween20, 4 μL 200 nM sortilin in 50 mM Bis-Tris Propane pH 8, 50 mM NaCl, 0.05% Tween20. The reaction was allowed to incubate for 2.5 hours at room temperature, at which point the HTRF signal ratio at 665/620 nm was determined using a plate reader (ClarioStar, BMG Labtech). All reactions were run in triplicates and data was represented as HTRF ratio as a function of bifunctional compound concentration.

FIG. 2A shows proximity induced HTRF ratio as function of compound concentration for two peptidic compounds. For a compound according to SEQ ID NO.: 147 a bell-shaped dose-response was observed, signifying relative low concentration of ternary complex formation at both low and high compound concentrations which is a common pharmacological profile observed for bifunctional molecules and is commonly termed the hook-effect. A compound according to SEQ ID NO.: 157 is identical to compound according to SEQ ID NO.: 147 in all aspect besides the addition of C-terminal amidation, and consequently this peptide did not induce dose-dependent increase in HTRF ratio, signifying that this peptide was not capable of mediating ternary complex formation between streptavidin and sortilin.

TABLE 2
AUC for TR-FRET measurements of ternary complex formation (AUC
< 5000: +; AUC > 5000: ++)

| SEQ ID NO.: | AUC score |
|---|---|
| 141 | + |
| 142 | + |
| 145 | + |
| 146 | ++ |
| 147 | ++ |
| 148 | ++ |
| 149 | + |
| 155 | ++ |

### Conclusion

Compounds of the invention can mediate ternary complex formation between sortilin and target proteins.

### Example 3: Internalization of Target Protein

Addressed in this example is the ability of sortilin to internalize an extracellular target of a bifunctional compounds wherein S_{L} is derived from a peptide. In this example, the peptidic degraders consist of a sortilin binder motif linked to a warhead of biotin supposed to target fluorescent NeutrAvidin-650 (NA650).

Cellular uptake of NA650 was investigated in HEK293 cells expressing full length sortilin receptor (Petersen et. al. [13]). Cells were seeded in poly-L-lysine coated 96-well plates (Perkin Elmer) (40K/well) in 50 μL culture medium (DMEM (Lonza)+10% FBS (SigmaAldrich)+1% penicillin-streptomycin (SigmaAldrich)+1% GlutaMAX (Gibco)+100 μg/mL Zeocin (Invitrogen)) and incubated overnight in cell incubator (37° C., humidified, 5% CO₂).

Culture medium was replaced with assay medium (DMEM (Lonza)+10% fetal bovine serum (SigmaAldrich)+1% penicillin-streptomycin (SigmaAldrich)+1% GlutaMAX (Gibco)) containing NA650 (Invitrogen) (100 nM) (Thermo Fischer) and peptidic bifunctional compounds (9.8 nM to 10 μM). Cells were incubated for 3 h in cell incubator before they were washed in dPBS (Bionordika). The DyLight650 signal in the cell layer was detected using a fluorescence plate reader (BMG Labtech Clariostar).

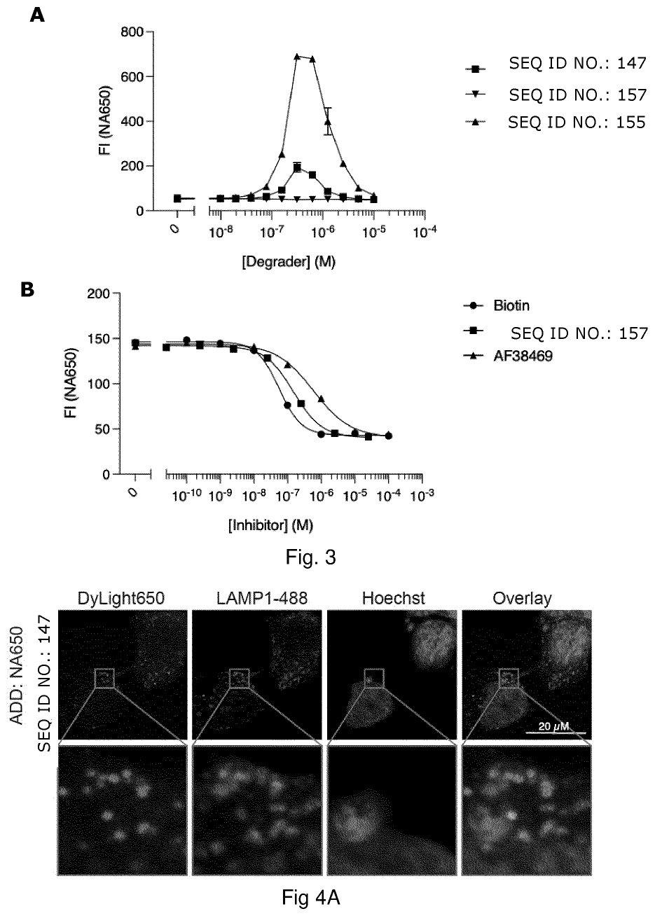

FIG. 3A shows cell associated FI signal following 3 h incubation of HEK293/sortilin with a concentration series of SEQ ID NO.: 147, SEQ ID NO.: 157 or SEQ ID NO.: 155 (9.8 nM to 10 μM) and NA650 (Invitrogen) (100 nM) (n=2). The bell-shaped response to increased concentration SEQ ID NO.: 147 and SEQ ID NO.: 157 is consistent with the hook effect commonly observed in systems involving ternary complex formation. Data points are shown as mean value+/−SEM.

FIG. 3B shows cell associated FI signal following 3 h incubation of HEK293/sortilin with SEQ ID NO.: 147 (300 nM) and NA650 (Invitrogen) (100 nM) in presence of small molecule sortilin binder AF38496 (Schrøder et al [8]) (0.1 nM-100 μM), SEQ ID NO.: 172, a peptide without biotin warhead (0.025 nM-25 μM), or biotin alone (0.1 nM-100 μM) (n=2). Data shows that degrader induced internalization of NA650 is inhibited by competitive binding to sortilin by AF38469 (IC50 589 nM) or RQLL peptide (IC50 149 nM) and competitive binding to target by biotin (IC50 56 nM). Data points are shown as mean value+/−SEM.

### Conclusion

This example shows that bifunctional compounds consisting of a sortilin binding motif linked to a biotin warhead mediates uptake of extracellular NA650. In addition, the provided data shows that internalization of target depends on the bifunctional compound binding to the sortilin receptor and target.

### Example 4: Target Molecule Sorting to Lysosomes for Degradation

### Aim

Addressed in this example is the ability of a bifunctional compound wherein S_{L} is derived from a peptide to mediate trafficking of an extracellular target via the endo-lysosomal pathway for degradation in the lysosomes. This was investigated by fluorescence microscopy evaluation of subcellular localization of internalized target and on-blot evaluation of intracellular target level following inhibition of lysosomal function.

### Materials and Methods

Cellular uptake of target NA650 was analyzed with fluorescence microscopy. For this assay, coverslips in 4 well plates were coated with poly-L lysine prior to seeding of HEK293/sortilin cells (25K cells/coverslip) in culture media (as described in Example 3)) and incubated overnight in cell incubator (37° C., humidified, 5% CO2). The culture media was replaced with assay media (as described in Example 3) containing NA650 (Invitrogen) (500 nM) (Thermo Fischer), a compound according to SEQ ID NO.: 147 (2 μM) and AF38469 (100 μM, MedChemExpress). The cells were incubated in a cell incubator for 4 hours before washing in dPBS (Bionordika) followed by fixation with dPBS+4% PFA (Sigma Aldrich) for 15 minutes. The cells were permeabilized in TBS+0.1% Triton X-100 (Sigma Aldrich) for 15 minutes before washing with dPBS and incubation with primary antibodies (LAMP-1 mAb H4A3, Abcam Ab25630) at 4° C. overnight. Incubation with secondary antibodies (Goat-anti-mouse Alexa-Fluor 488 (ThermoFisher)) was carried out for 2 hours at room temperature before washing in dPBS and incubation in Hoechst (1:10.000, Sigma Aldrich) for 10 minutes. The coverslips were dipped shortly in 70% ethanol (VWR) and mounted on a glass slide with fluorescent mounting medium (Agilent Dako). The fluorescent signal was analyzed using a laser scanning confocal microscope (LSM-800).

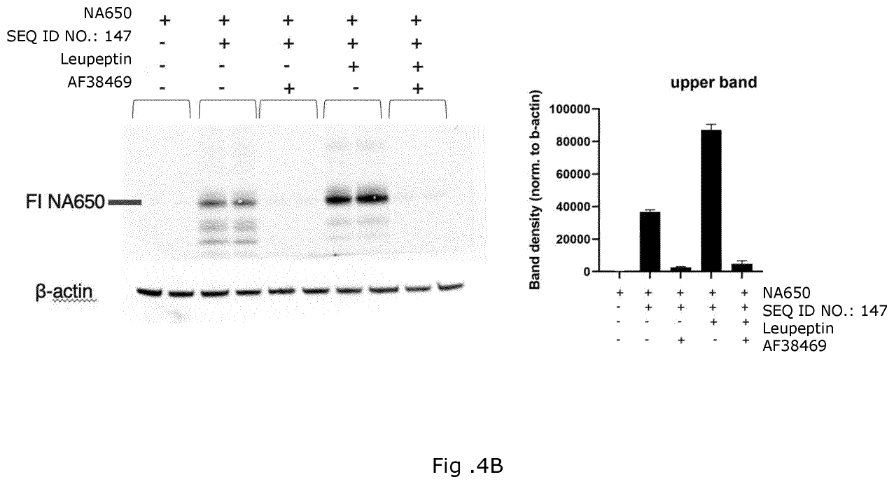

FIG. 4A provides fluorescence microscopy imaging data demonstrating that target NA650 (DyLight650) co-localizes with lysosomal marker in HEK293/sortilin cells fixed and immuno-stained with anti-LAMP1 (Alexa-488) following 2 h incubation with bifunctional molecule SEQ ID NO.: 147 (300 nM) and NA650 (100 nM). Nucleus stained with Hoechst. Scale bar is 20 μM

Intracellular levels of NA650 following inhibition of lysosomal function, were investigated in HEK293/sortilin cells. Cells were seeded in poly-L-lysine coated 4-well plates (Thermo Fisher) (250K/well) in 250 UL culture medium (as described in Example 3) and incubated overnight in cell incubator.

Culture medium was replaced with assay medium (DMEM (Lonza), 10% fetal bovine serum (Sigma Aldrich), 1% penicillin-streptomycin (Sigma Aldrich), 1% GlutaMAX (Gibco)) with or without NA650 (Invitrogen) (100 nM) (Thermo Fischer), peptidic bifunctional compound (300 nM) and leupeptin (80 μM) (Sigma Aldrich). Cells were incubated 24 h in cell incubator before they were washed with dPBS (Bionordika) and lysed in lysis buffer (STE buffer (Sigma Aldrich)+1% Nonidet™ P 40 Substitute (SigmaAldrich)+cOmplete (Roche)). Lysates were centrifugated and the supernatants were mixed with LDS sample buffer (Thermo Fisher) and DTT (Sigma Aldrich) and boiled for 5 minutes before proteins were separated on a 4-12% Bis-Tris gel (Thermo Fisher). Proteins were blotted onto a nitrocellulose membrane (Thermo Fisher), and the DyLight650 signal was detected using an iBright 1500 (Thermo Fisher).

Membranes were blocked for 1 h in blocking buffer (TBS (Fisher Scientific) with 1% Tween20 (Sigma Aldrich) and 5% milk powder) followed by incubation with primary anti-β actin antibody (1:3500) (SigmaAldrich) for 2 h at room temperature. The blot was washed 3 times in wash buffer (TBS (Fisher Scientific)+1% Tween20 (Sigma Aldrich)+0.5% milk powder) and incubated with secondary anti-mouse-HRP antibody (1:3500) (Abcam) for 1 h room temperature before it was washed 3 times in wash buffer and the blot was developed and analyzed using ECL reagents (Cytiva) and iBright 1500 (Thermo Fisher).

FIG. 4B provides data further demonstrating intracellular accumulation of target upon inhibition of lysosomal cysteine protease activity by addition of Leupeptin (80 μM). In the shown example, cells were incubated with 100 nM NA650 (Invitrogen), peptidic bifunctional compound (300 nM) and leupeptin (80 μM) (Sigma Aldrich) for 24 h prior harvest of cell-lysates and separation of proteins on SDS-PAGE gel. Bar graph shows quantification of NA650 upper band (mean value with std).

### Conclusion

This example shows that a target of a bifunctional compound is sorted form the extracellular space to lysosomal compartments for subsequent degradation.

### Example 5: Depletion of Target Molecules from Extracellular Media

### Aim

This example investigates if a bifunctional compound wherein S_{L} is derived from a peptide can deplete a target from the extracellular space as addressed by evaluation of fluorescent intensity (FI) in cell culture supernatant following incubation of cells with the bifunctional compound and NA650.

### Materials and Methods

HEK293/sortilin cells were cultured in culture media containing DMEM (Lonza) further supplemented with 10% fetal bovine serum (Sigma), 1% GlutaMax Supplement (Gibco), penicillin-streptomycin (Thermo Fischer) and selection antibiotics 100 μg/mL Zeocin (Invitrogen). 35k cells/well were seeded in poly-L-lysine coated 96-well plates (Perkin Elmer) and allowed to incubate for 16-24 h in cell incubator (37° C., humidified, 5% CO₂). Culture media were discarded, and the cells were washed with assay medium containing FluoroBright DMEM (Gibco) supplemented with 10% fetal bovine serum (Sigma) and 1% GlutaMax Supplement (Gibco). The media were discarded and replaced with assay media or assay medium containing 100 nM NA650 (Invitrogen). For wells evaluating the bifunctional compounds, the assay medium also contained concentrations ranging from 20 nM to 5 μM. After an incubation period of up to 72 h in the cell incubator the media were harvested and the presence of NA650 were evaluated by fluorescence intensity (FI) measurements of DyLight650 using a fluorescence plate reader (BMG Labtech Clariostar).

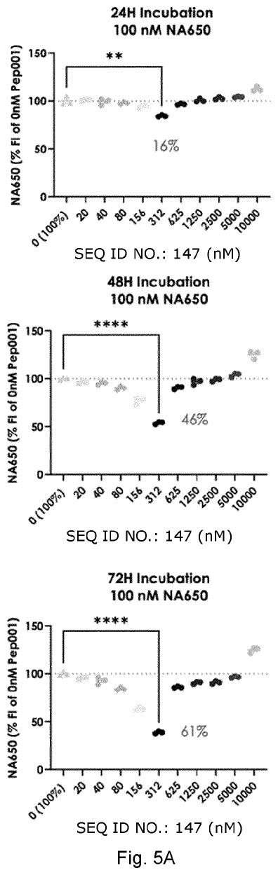

FIG. 5A shows NA650 signal in HEK293/sortilin cell culture supernatant following incubation with SEQ ID NO.: 147 (20 nM to 10 μM) and NA650 (Invitrogen) (100 nM) for up to 72 h. Single addition of the bifunctional compound results in a significant reduction in signal after 24 h (16%), 48 h (46%) and 72 h (61%) incubation. For all timepoints, the highest uptake of target is observed at 312 nM degrader.

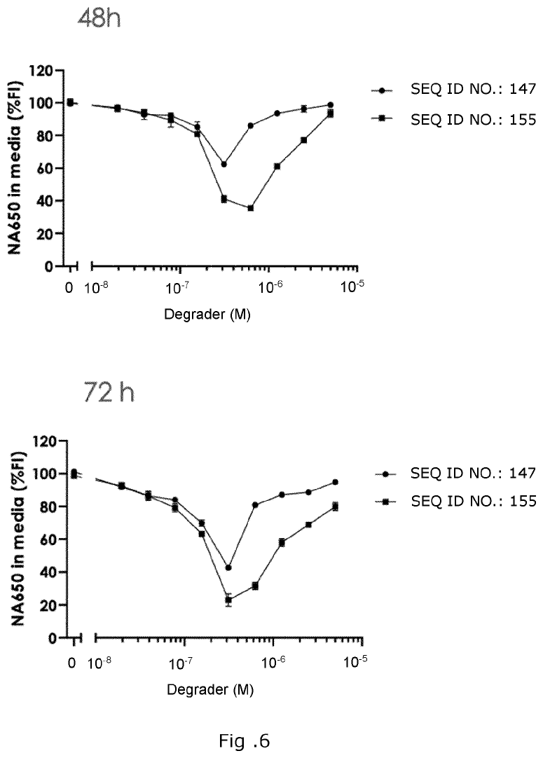

FIG. 6 shows NA650 FI signal in HEK293/sortilin cell culture supernatant following incubation with NA650 (Invitrogen) (100 nM) and SEQ ID NO.: 147 (20 nM-5 μM) or SEQ ID NO.: 155 (20 nM to 5 M), a bifunctional compound with improved affinity for sortilin, resulting in further reduction of target in cell culture supernatant compared to SEQ ID NO.: 147 after 48 and 72 hours.

### Conclusion

This example demonstrates that target concentration in media decreases over time showing bifunctional compound-mediated depletion of target and further suggesting a sustainable mechanism of action.

### Example 6: Binding of Peptides

### Aim

In this example the equilibrium dissociation constant (KD) describing the binary binding event between a peptide and the soluble ectodomain of sortilin-6his is measured by microscale thermophoresis (MST).

### Materials and Methods

MST was performed as described in example 1.

TABLE 3
Binding to sortilin (Kd) (K_{D} score: K_{D} <25 nM:
+++; K_{D} 25-250 nM: ++;
K_{D} 250-2500: +; K_{D} >2500 nM: 0).

| SEQ ID NO.: | Sequence | KD score |
|---|---|---|
| 22 | H-RQLL-OH | ++ |
| 163 | H-RQLI-OH | 0 |
| 164 | H-RQLF-OH | 0 |
| 165 | H-RQLM-OH | 0 |
| 166 | H-RQL-OH | 0 |
| 23 | H-FQLL-OH | ++ |
| 24 | H-KQLL-OH | ++ |
| 25 | H-PQLL-OH | ++ |
| 47 | H-RLFKKRRLRSPRVLF-NH2 | 0 |
| 48 | H-ITVDPRLFKKRRLRSPRVLF-NH2 | 0 |
| 167 | GGSGGGGSGGGGSGGRQLL-OH | ++ |
| 26 | H-PYIL-OH | ++ |
| 27 | H-RYLL-OH | ++ |
| 28 | H-FYLL-OH | ++ |
| 29 | H-YQLL-OH | ++ |
| 30 | H-LQLL-OH | ++ |
| 31 | H-FYIL-OH | + |
| 32 | H-RYIL-OH | + |
| 33 | H-PYLL-OH | + |
| 36 | H-RYYL-OH | ++ |
| 37 | H-RYFL-OH | + |
| 38 | H-RQFL-OH | + |
| 39 | H-RQYL-OH | + |
| 52 | H-GVSWGLR-OH | 0 |
| 53 | REAPRWDAPLRDPALRQLL | +++ |
| 54 | REALRWDAPLRDP APRQLL | +++ |
| 55 | PYILKRQLYENKPRRPYIL | +++ |
| 62 | TGGFM | 0 |
| 63 | YGGFL | 0 |
| 56 | LYENKPRRPYIL | ++ |
| 57 | APLRDPAPRQLL | ++ |
| 58 | REAPRWDAPLRDPALFQLL | +++ |
| 59 | REAPRWDAPLRDPALRYLL | +++ |
| 61 | REAPRWDAPLRDPALRYYL | ++ |
| 43 | FYYL | 0 |
| 64 | LYEN-NH2 | 0 |
| 65 | LYENK-NH2 | 0 |
| 40 | LQLL | + |
| 41 | HQLL | 0 |
| 42 | IQLL | + |
| 179 | WSGPIGVSWGLRAAAAGFQLL-OH | +++ |
| 196 | AARL-OH | + |
| 197 | PIPLV-OH | 0 |

### Conclusion

Assessed in this example is whether a monofunctional peptide binds directly to the soluble ectodomain of sortilin and quantification the binding event by measuring the dissociation constant at equilibrium.

### Example 7: Conjugation of Sortilin-Binding Peptides to Antibodies

### Aim

In this example the monoclonal antibody alirocumab (anti-PCSK9, light chain SEQ ID NO.: 67, heavy chain SEQ ID NO.: 66) or adalimumab (anti-TNFalpha, light chain SEQ ID NO.: 69, heavy chain SEQ ID NO.: 68) is covalently conjugated with a peptide, composed in one end of an NHS group linked to the C-terminal sortilin binding peptide sequence RQLL hereby generating the bifunctional alirocumab-link-RQLL (SEQ ID NO.: 159) or adalimumab-link-RQLL (SEQ ID NO.: 161). A negative control sample is generated by conjugating alirocumab with an identical peptide as described above in all aspects besides lacking the C-terminal peptide sequence RQLL thereby generating the sample alirocumab-link. (SEQ ID NO.: 160) or adalimumab-link (SEQ ID NO.: 162).

### Materials and Methods

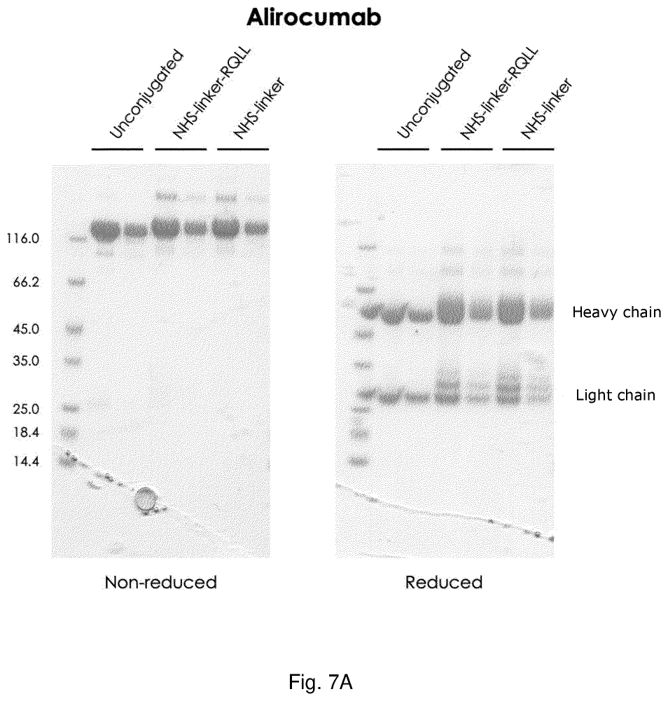

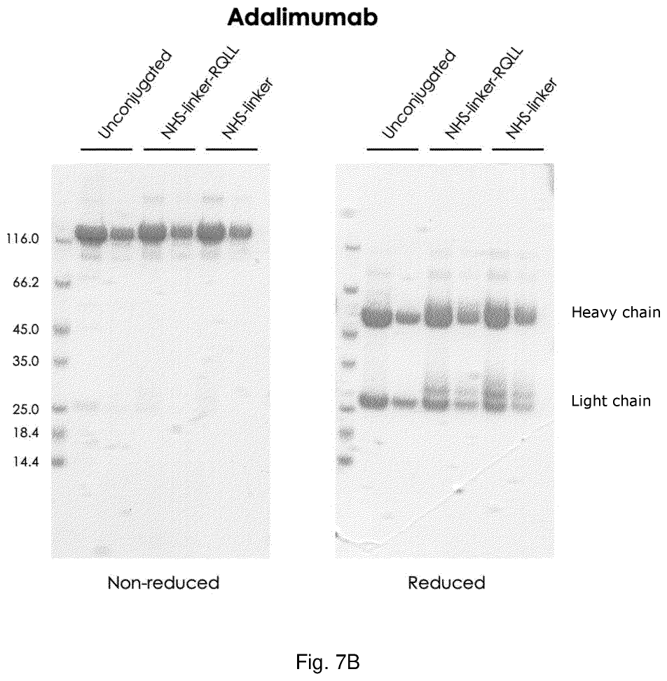

NHS containing peptides NHS-PEG4-Mal-CGGSGGGGSGGGGSGGRQLL (SEQ ID NO.: 168) and NHS-PEG4-Mal-CGGSGGGGSGGGGSGG SEQ ID NO.: 169 were solubilised to 5 mM in DMSO and added in 25 molar excess to a 4 mg/ml solution of antibody in PBS. The reactions were incubated at room temperature for 24 hours at which point unreacted NHS containing peptide was removed by applying the sample to a 2 mL Zeba spin desalting column. Following this protocol two separate samples was prepared i.e., antibody-link-RQLL and antibody-link. The reactions were followed by SDS-PAGE analysis as shown in FIG. 7A and FIG. 7B

FIG. 7A shows the SDS-PAGE analysis of conjugation of NHS-linker (SEQ ID NO.: 169) or NHS-linker-RQLL (SEQ ID NO.: 168) to Alirocumab under non-reduced or reduced (2 mM DTT+heating) conditions. Successful conjugation of NHS-linker and/or NHS-linker-RQLL for Alirocumab was observed, as can be seen from the mass shift when compared to unconjugated antibodies

FIG. 7B shows the SDS-PAGE analysis of conjugation of NHS-linker or NHS-linker-RQLL to Adalimumab under non-reduced or reduced (2 mM DTT+heating) conditions. Successful conjugation of NHS-linker (SEQ ID NO.: 169) and/or NHS-linker-RQLL (SEQ ID NO.: 168) for Adalimumab was observed, as can be seen from the mass shift when compared to unconjugated antibodies.

### Conclusion

Compounds of the invention can be covalently coupled to antibodies

### Example 8: Bifunctional Compounds Derived from Antibody Conjugates Bind Sortilin with nM Affinity

### Aim

In this example the equilibrium dissociation constant (KD) describing the binary binding event between a monoclonal antibody conjugated to a sortilin binder (SEQ ID NO.: 159) and the soluble ectodomain of sortilin-6his is measured by microscale thermophoresis (MST)

### Materials and Methods

MST binding experiments of alirocumab-link-RQLL (SEQ ID NO.: 159) or alirocumab-link (SEQ ID NO.: 160) solutions was performed in a protocol adapted from example 1.

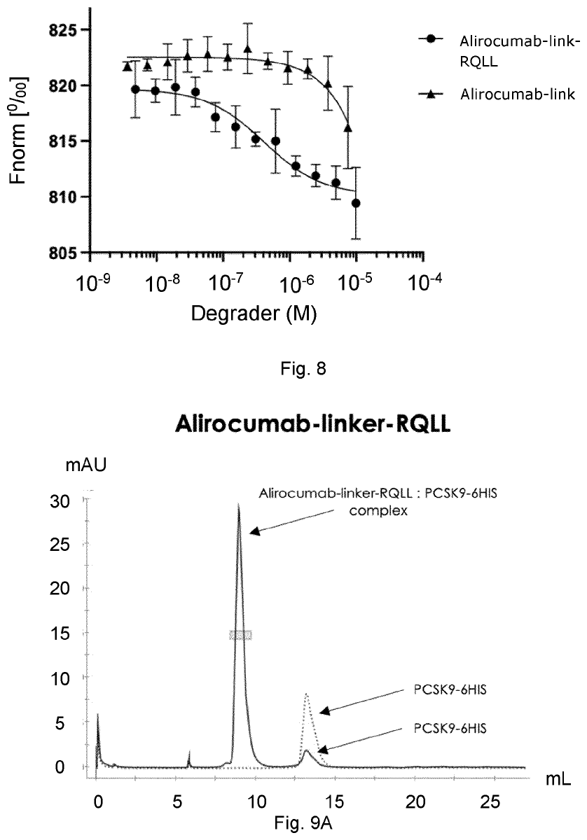

FIG. 8: shows MST binding response as function of antibody concentration. From this experiment a clear sigmoidal dose-response was observed for alirocumab conjugated with a peptide containing the sortilin binding sequence (SEQ ID NO.: 159 Alirocumab-link-RQLL) resulting in a dissociation constant of 540 nM while alirocumab conjugated with a peptide lacking the C-terminal sortilin binding sequence (Alirocumab-link, SEQ ID NO.: 160) did not produce a sigmoidal binding curve signifying that this antibody does not bind sortilin with measurable dissociation constant.

### Conclusion

Covalent coupling of compounds of the invention to antibodies results in sortilin binding.

### Example 9: Bifunctional Compounds Derived from Antibodies Retain Binding to Antigen

### Aim

In this example we further assessed if alirocumab-linker-RQLL and alirocumab-link retain binding capabilities to PCSK9-6HIS by gel-filtration.

### Materials and Methods

Alirocumab-link-RQLL (SEQ ID NO.: 159) or alirocumab-link (SEQ ID NO.: 160) was mixed with PCSK9-6HIS (SEQ ID NO.: 171) in a 1:2.5 molar ratio (1.67 μM Alirocumab and 4.175 μM PCSK9-6HIS) in a final volume of 200 uL and allowed to incubate 24 hrs at 4° C. Next day the samples were spun down 15 min at 13400 rpm at 4° C. to remove potential aggregates and the samples were applied to a Superdex200 increase 10/300 (Cytiva) connected to a Äkta Pure HPLC system (Cytiva). In each experiment, 0.5 mL fraction were collected and analyzed by SDS-PAGE (reducing and non-reducing). A negative control (PCSK9-6his alone) was produced following similar procedure as described above.

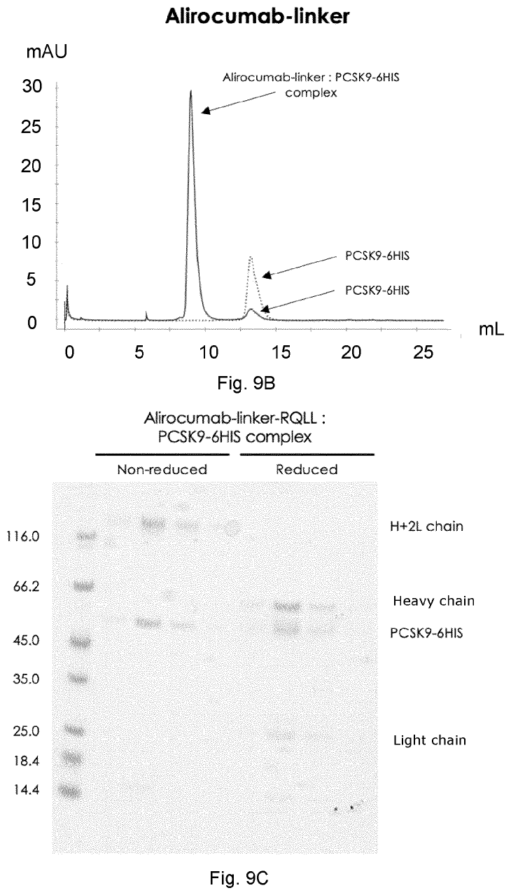

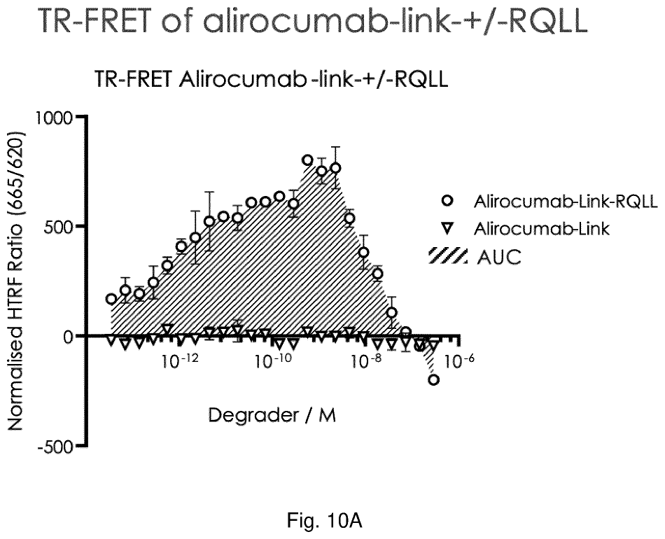

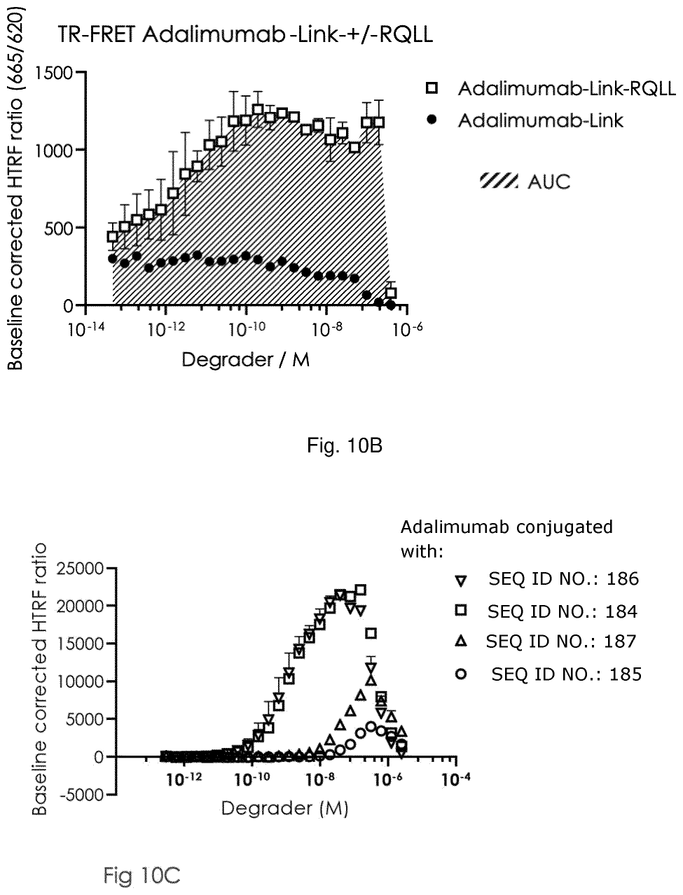

FIG. 9 shows that a complex between PCSK9-6HIS and alirocumab-linker-RQLL (FIG. 9A) or alirocumab-linker (FIG. 9B) was formed and could be purified by gel-filtration. This is evident as both a reduced peak size for PCSK-6HIS and co-elution with alirocumab-linker-RQLL is shown in the FIG. 9C SDS-PAGE analysis of peak fractions from the protein complex.

### Conclusion

Complex formation between target and a) alirocumab-linker-RQLL (SEQ ID NO.: 159) or b) alirocumab-linker (SEQ ID NO.: 160) was formed and could be purified by gel-filtration. This is evident as both a reduced peak size for PCSK-6HIS and co-elution with alirocumab-linker-RQLL is shown in the c) SDS-PAGE analysis of peak fractions from the protein complex.

### Example 10: Ternary Complex Formation with Bifunctional Compounds Derived from Antibodies

### Aim

Addressed in this example is the ability of monoclonal antibodies conjugated to sortilin binding peptides to mediate the formation of a ternary protein complex with the target protein and sortilin.

### Materials and Methods

In these experiments, bifunctional compounds alirocumab-link-RQLL (SEQ ID NO.: 159) and adalimumab-link-RQLL (SEQ ID NO.: 161) were titrated in 4 μL total volume over 24-point in a 384-well TTP LVDS plate (SPT Labtech) to a final buffer composition of 50 mM Bis-Tris Propane pH 8, 50 mM NaCl, 0.05% Tween20 using a Mosquito LV pipetting robot (SPT Labtech). The same test was performed with antibody conjugates without the binding motif (alirocumab-link SEQ ID NO.: 160 and adalimumab-link SEQ ID NO.: 162).

PCSK9 target: To a black 384-well plate (Corning) the following were added: 4 μL 10 nM mAb anti-6HIS Tb (Cisbio) in 50 mM Bis-Tris Propane pH 8, 50 mM NaCl, 0.05% Tween20, 4 μL 250 nM Streptavidin-d2 (Cisbio) in 50 mM Bis-Tris Propane pH 8, 50 mM NaCl, 0.05% Tween20, 4 μL 200 nM Sortilin-6His in 50 mM Bis-Tris Propane pH 8, 50 mM NaCl, 0.05% Tween20, 4 μL 200 nM Biotin-avi-PCSK9 in 50 mM Bis-Tris Propane pH 8, 50 mM NaCl, 0.05% Tween20 and 4 μL alirocumab-link-RQLL (SEQ ID NO.: 159) or alirocumab-link (SEQ ID NO.: 160) in 50 mM Bis-Tris Propane pH 8, 50 mM NaCl, 0.05% Tween20

TNF-α target: To a black 384-well plate (Corning) the following were added: 4 μL 10 nM mAb anti-6HIS Tb (Cisbio) in 50 mM Bis-Tris Propane pH 8, 50 mM NaCl, 0.05% Tween20, 4 μL 250 nM Streptavidin-d2 (Cisbio) in 50 mM Bis-Tris Propane pH 8, 50 mM NaCl, 0.05% Tween20, 4 μL 1000 nM Sortilin-6His in 50 mM Bis-Tris Propane pH 8, 50 mM NaCl, 0.05% Tween20, 4 μL 200 nM TNF-alpha-Biotin in 50 mM Bis-Tris Propane pH 8, 50 mM NaCl, 0.05% Tween20 and 4 μL adalimumab-link-RQLL (SEQ ID NO.: 161) or adalimumab-link (SEQ ID NO.: 162) in 50 mM Bis-Tris Propane pH 8, 50 mM NaCl, 0.05% Tween20

After incubation for 2.5 hours at room temperature the HTRF signal ratio 665/620 nm was determined on a ClarioStar plate reader (BMG Labtech). All reactions were run in duplicates and data was represented as HTRF ratio as a function of bifunctional compound concentration.

FIG. 10A shows the HTRF (665/620 nm) ratio normalised to alirocumab-Link plotted against a concentration gradient of alirocumab conjugated with linker and with or without RQLL (290 nM-3.5 fM) following 2.5 hrs incubation. This shows that ternary complex formation, as visualised by a bell-shaped curve, is dependent on the RQLL motif. (Error bars=S.D.; n=2)

FIG. 10B shows the resulting HTRF (665/620 nm) signal baseline corrected to the lowest global signal plotted against a concentration gradient of adalimumab conjugated with linker and with or without RQLL (400 nM-4.7 fM) after 2.5 hrs incubation. This shows that ternary complex formation, as visualised by a bell-shaped curve, is dependent on the RQLL motif. (Error bars=S.D.; n=3)

FIG. 10C shows the resulting HTRF (665/620 nm) signal baseline corrected to the lowest global signal plotted against a concentration gradient of adalimumab conjugated with sortilin binding peptides SEQ ID NO.: 184 to SEQ ID NO.: 187 (400 nM-4.7 fM) after 2.5 hrs incubation. This shows ternary complex formation, as visualised by a bell-shaped curve (Error bars=S.D.; n=3)

### Conclusion

Compounds of the invention mediate ternary complex formation between sortilin and target.

### Example 11: Bifunctional Compounds Derived from Antibodies Mediate Uptake of Extracellular Targets

### Aim

Addressed in this example is the ability of sortilin to facilitate internalization of an extracellular target of a monoclonal antibody conjugated with a sortilin binding peptide.

### Materials and Methods

In this example is presented bifunctional antibody conjugates targeting PCSK9 alirocumab-link-RQLL (SEQ ID NO.: 159) and TNF-α adalimumab-link-RQLL (SEQ ID NO.: 161).

Cellular uptake was measured as in Example 3 using Cy5 (Thermo Fisher) labelled target (100-200 nM).

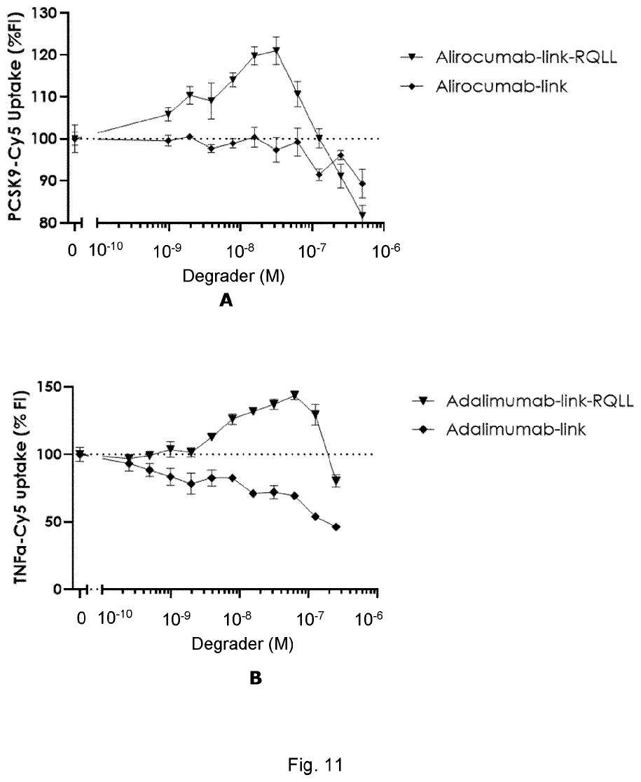

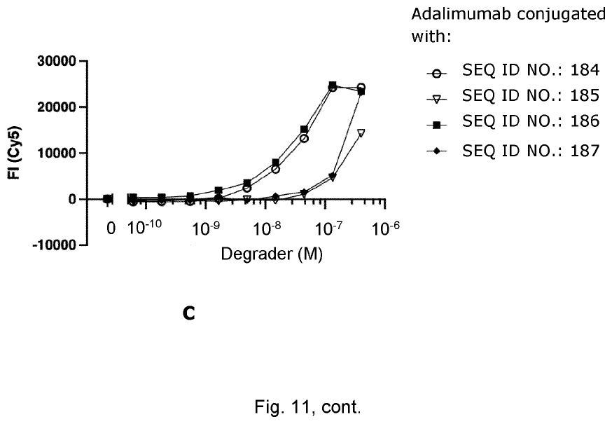

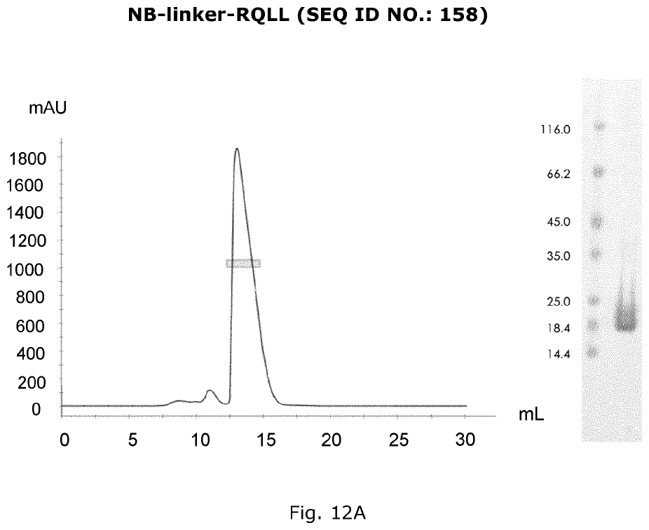

FIG. 11A shows cell associated FI signal following 3 h incubation of HEK293/sortilin with a concentration series of alirocumab-link-RQLL (SEQ ID NO.: 159) (1 nM to 0.5 M) and Cy5-PCSK9 (200 nM). In a control experiment, cells were incubated with alirocumab with a peptide lacking the C-terminal sortilin binding sequence alirocumab-link (SEQ ID NO.: 160).

FIG. 11B shows cell associated FI signal following 3 h incubation of HEK293/sortilin with a concentration series of adalimumab-link-RQLL (SEQ ID NO.: 161) (250 pM to 250 nM) and TNFalpha (100 nM). In a control experiment, cells were incubated with adalimumab with a peptide lacking the C-terminal sortilin binding sequence adalimumab-link (SEQ ID NO.: 162).

FIG. 11C shows cell associated FI signal following 3 h incubation of HEK293/sortilin with a concentration series of adalimumab conjugated with sortilin binding peptides of SEQ ID NO.: 184 to SEQ ID NO.: 187 (250 pM to 250 nM) and TNFalpha (100 nM).

### Conclusion

This example shows that a monoclonal antibody conjugated to a sortilin binding peptide, can mediate uptake of an extracellular disease protein e.g. PCSK9 or TNFalpha.

### Example 12: Production of Anti-LC VHH Production

### Aim

In this example a bifunctional nanobody conjugated to a sortilin binding peptide is produced, capable of binding to both kappa-light chain antibodies and sortilin (NB-linker-RQLL, SEQ ID NO.: 158). A control without the RQLL motif is also prepared (NB-linker, SEQ ID NO.: 71).

### Materials and Methods

The plasmids (Nanobody-pET11d: 28-B09 (SEQ ID NO.: 177); Nanobody-delRQLL:pET11d:23-F06, Genscript (SEQ ID NO.: 173)) expressing the bispecific nanobodies were transformed into the E. coli strain Shuffle-T7-Express-Lys-Y (NEB) and plated on plates containing 100 microgram/ml ampicillin following incubation at 37° C. for 18 hours. Overnight cultures were grown in 100 ml LB medium (Luria-Bertani) at 30° C. and 100 microgram/ml ampicillin and at 175 RPM. Cell pellets from overnight cultures were resuspended in 1 liter of 2×YT medium (Tryptone 16 gr/l, Yeast Extract 10 gr/l, NaCl 5 gr/l, 100 microgram/ml ampicillin) and incubated at 30° C. and at 200 RPM. At an OD600 of 0.75, IPTG (Isopropyl ß-D-1-thiogalactopyranoside) was added to final concentration 0.4 mM. Cultures were then incubated at 16° C. and at 200 RPM for 18 hours. Cell pellets were harvested at 6000 RPM for 15 min and kept at −20° C.

After harvesting, cells were resuspended in 20 mM Tris-HCl pH 8.0, 500 mM NaCl, 20 mM Imidazole, 1 mM PMSF and lysed by sonication. The lysate was clarified by centrifugation and supernatant was loaded onto a 5 mL HisTrap FF Crude column (Cytiva) equilibrated in 20 mM Tris-HCl pH 8.0, 500 mM NaCl, 20 mM Imidazole. The loaded column was washed to baseline and elution was performed with a linear gradient from 20-300 mM Imidazole. Protein was dialysed (MWCO: 6-8 kDa) against 20 mM Sodium acetate pH 5.5, 50 mM NaCl O/N at 4° C. followed by concentration in a centrifugal concentrator (MWCO: 5 kDa). The concentrated sample was subjected to size-exclusion chromatography on a Superdex75 Increase column (Cytiva) equilibrated in 25 mM Tris-HCl pH 7.4, 150 mM NaCl. Pure protein sample was concentrated, flash-frozen and stored at −80° C.

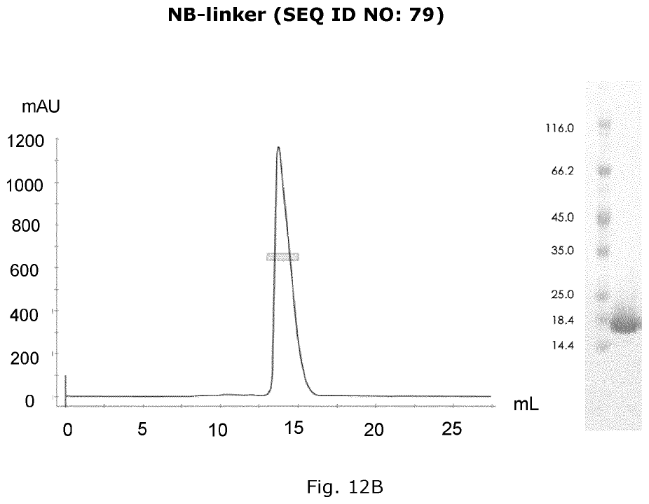

FIG. 12 shows the size-exclusion chromatography elution profile and final sample of A) NB-linker-RQLL, SEQ ID NO.: 158 and B) NB-linker, SEQ ID NO.: 71. Pooled and concentrated fractions from size-exclusion are indicated with a grey box.

### Example 13: Nanobody Derived Bifunctional Compound Binds Target Molecules

### Aim

In this example we used gel-filtration to assess binding capacities of NB-linker-RQLL, SEQ ID NO.: 158 and NB-linker, SEQ ID NO.: 71 to an antibody containing kappa light chains. Alirocumab is used as tool antibody with kappa light chains in the example.

### Materials and Methods

NB-linker-RQLL, SEQ ID NO.: 158 and NB-linker, SEQ ID NO.: 71 were mixed with tool antibody in a 2.2:1 molar ratio in a final volume of 250 uL and allowed to incubate 1 hr at RT. The samples were spun down 15 min at 13400 rpm at 4° C. to remove potential aggregates and the samples were applied to a Superdex75 Increase 10/300 (Cytiva) connected to a Äkta Pure HPLC system (Cytiva). In each experiment, 0.5 mL fractions were collected and analyzed by SDS-PAGE. A negative control (NB-linker alone) was produced following similar procedure as described above.

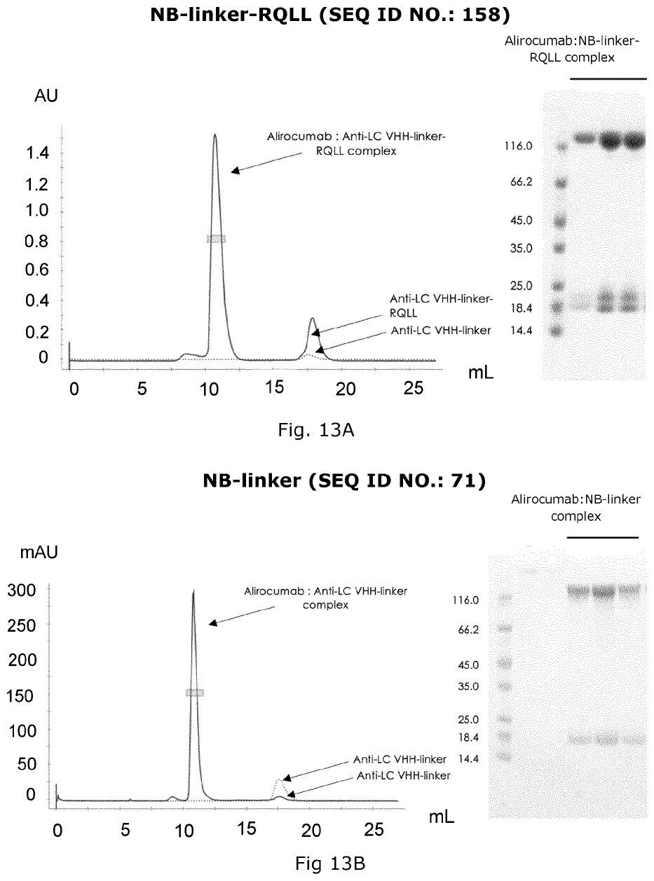

FIG. 13 shows that complex formation between IgG target and (FIG. 13A) NB-linker-RQLL, SEQ ID NO.: 158 or (FIG. 13B) NB-linker, SEQ ID NO.: 71 was formed and could be purified by gel-filtration. The SDS-PAGE analysis of the main peak confirms the presence of a complex with the target protein. Pooled and concentrated fractions from size-exclusion are indicated with a grey box.

### Conclusion

NB-linker-RQLL, SEQ ID NO.: 158 can bind and form complex with target IgG. The complex can be separated by size exclusion chromatography and confirmed by analysis in SDS-PAGE demonstrating co-elution of the target protein and the nanobody bifunctional compound.

### Example 14: Lysosomal Degradation of Extracellular Target IgG

### Aim

This example addresses cellular uptake and degradation of an extracellular target by a nanobody carrying the sortilin binding peptide sequence RQLL and a human IgG kappa-light chain binding sequence (NB-linker-RQLL, SEQ ID NO.: 158).

### Materials and Method

Cellular uptake of Cy5 conjugated IgG was investigated in HEK293 cells expressing sortilin receptor using an analogous methodology as described in Example 3 . . .

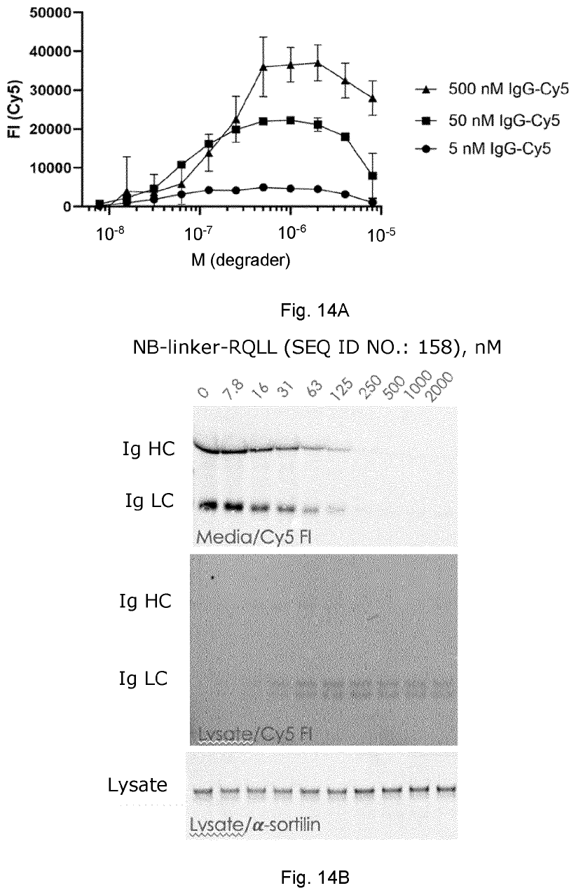

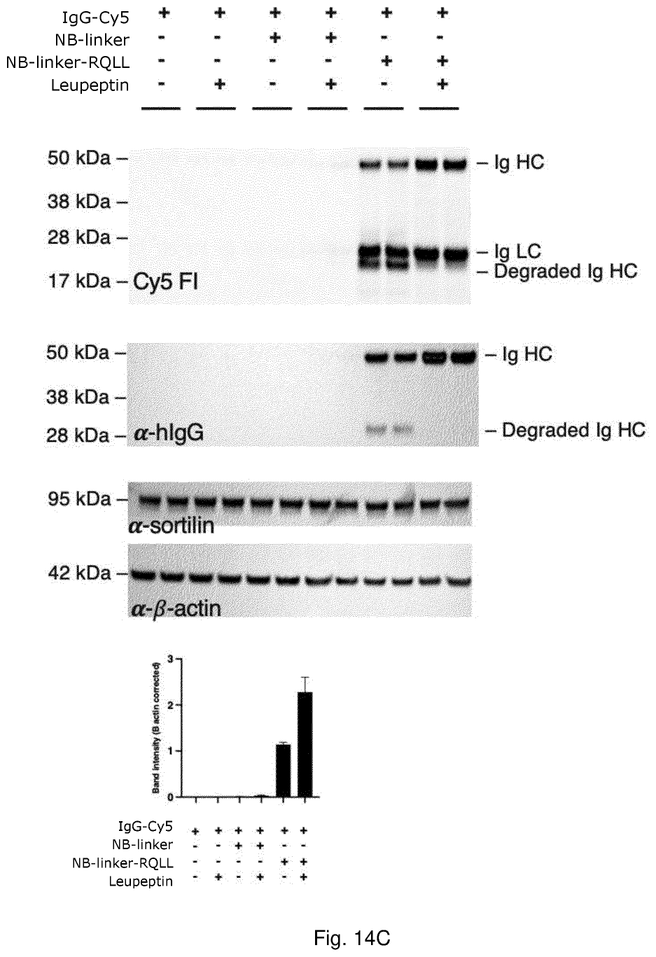

FIG. 14A shows cell associated FI signal following 3 h incubation of HEK293/sortilin with a concentration series of NB-linker-RQLL, SEQ ID NO.: 158 (8 nM to 8 μM) and Cy5 conjugated IgG (5, 50 or 500 nM).

In a different experiment, 35K or 250K cells were seeded in 4-well or 96 well plates (Thermo Fisher), in 100 or 250 μL culture medium (as described in Example 3) and incubated overnight in cell incubator (37° C., humidified, 5% CO2).

Culture medium was replaced with assay medium as described in Example 3)) containing SEQ ID NO.: 158 or control SEQ ID NO.: 71 (0 to 2 M) and Cy5 conjugated IgG (50 nM). A subset of samples were, in addition, added leupeptin (80 μM) to inhibit lysosomal proteases.

Cells were incubated in cell incubator (up to 72 h) before collection of cell supernatant and harvest and lysis of cells (as described in example 4). Lysates and media were subjected to SDS-PAGE (as described in Example 4). Proteins were blotted onto a nitrocellulose membrane (Thermo Fisher), and the Cy5 signal was detected using an iBright 1500 (Thermo Fisher). Alternatively, IgG was detected by Western blotting (as described in Example 4) using anti-human IgG antibody (l: 3500, Abcam) HRP conjugated anti-rabbit-IgG (1:3500, Abcam).

Following anti-human-IgG Western Blotting, the membrane was probed with mouse anti-human-β-Actin (1:3500, Sigma-Aldrich) HRP conjugated anti-mouse-IgG (1:3500, Abcam). β-Actin blot was used as loading control.

Subsequently, the membrane was stripped in Restore stripping buffer (Thermo Scientific) for 15 min at 37° C., and reprobed with anti-human-NTR3 (sortilin) Ab (1:3500, BD Bioscience) followed by incubation with HRP conjugated anti-mouse-IgG (1:3500, Abcam) and developed and analyzed using ECL reagents (Cytiva) and iBright 1500 (Thermo Fisher).

FIG. 14B shows Cy5 FI in cell culture supernatant (upper blot) and lysates (middle blot) of HEK293/sortilin cells following 72 h incubation with Cy5 conjugated IgG (kappa-light chain) (50 nM) and NB-linker-RQLL, SEQ ID NO.: 158 as indicated. The intensity of bands corresponding to IgG heavy chain (lg HC) and light chain (lg LC) decreases in media when target is co-incubated with increasing concentration of nanobody. IgG bands are undetectable in conditioned medium from cells incubated with highest nanobody concentration indicating complete ablation of target from media. In corresponding lysate samples are detected a weak Ig LC signal and a degradation product of lower weight. No Ig HC signal is observed in cell lysates, indicating that internalized IgG target is rapidly degraded after internalization. Lower blot confirms sortilin expression in cells by Western blotting.

FIG. 14C shows Cy5 FI (upper blot) and Western blot of IgG signal in lysates of HEK293/sortilin cells following 6:30 h incubation with Cy5 conjugated IgG (50 nM), NB-linker-RQLL, SEQ ID NO.: 158 or control nanobody (NB-linker, SEQ ID NO.: 71) (250 nM), and lysosomal protease inhibitor leupeptin (80 μM) as indicated (n=2). Upon incubation with leupeptin is observed increased intensity of bands corresponding to Ig HC and Ig LC. In addition, no degradation products are observed. All together indicating that IgG is degraded in lysosomes after internalization. The two lower blots confirm loading of gel (anti-β-actin) and sortilin expression in cells by Western blotting. Bar graph shows quantification of Ig HC as mean value+/−SEM.

### Conclusion

Anti-LC nanobody derived bifunctional compound mediates cellular uptake and protein degradation of extracellular IgG via lysosomal degradation.

### Example 15: Binding of Small Molecule Bifunctional Compounds to Sortilin

### Aim

In this example the equilibrium dissociation constant (KD) describing the binary binding event between different compounds and the soluble ectodomain of sortilin-6his is measured by microscale thermophoresis (MST).

### Materials and Methods

MST was performed as described in example 1.

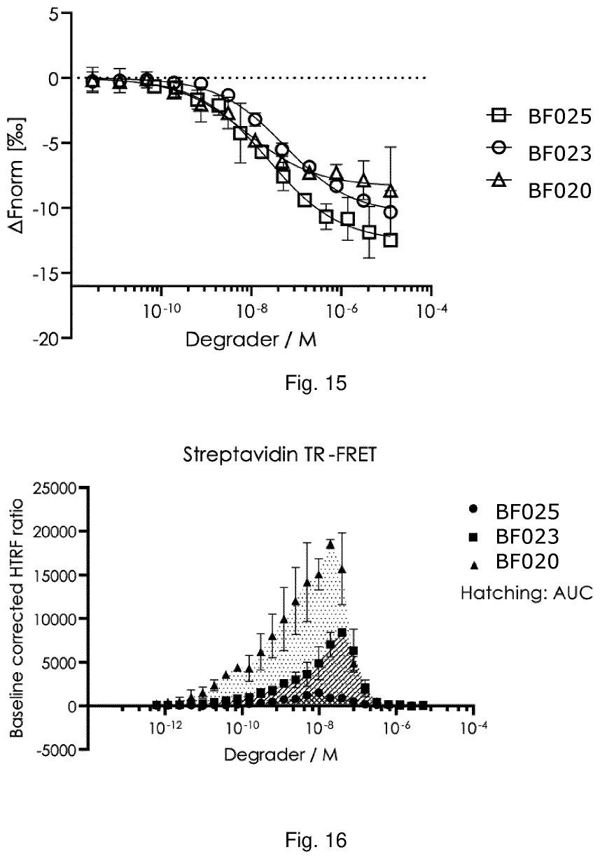

FIG. 15 shows of binding of BF025, BF023 and BF020 titrated (top concentration 12.5 μM) with constant NTA sortilin-6His (100 nM) in MST analysis. (Error bars=S.D.; n=2)

TABLE 4
Binding to sortilin (Kd) (K_{D} scores - K_{D} < 1 nM: ++++;
K_{D} 1-10 nM: +++; K_{D} 10-250 nM: ++; K_{D} > 250 nM: +; No binding: 0)

| Compound | K_{D} score | Compound | K_{D} score |
|---|---|---|---|
| BF001 | +++ | BF038 | + |
| BF002 | +++ | BF039 | +++ |
| BF003 | +++ | BF040 | + |
| BF004 | +++ | BF041 | ++++ |
| BF005 | +++ | BF042 | +++ |
| BF006 | +++ | BF043 | ++++ |
| BF007 | +++ | BF044 | ++ |
| BF008 | +++ | BF045 | +++ |
| BF009 | +++ | BF046 | ++++ |
| BF010 | +++ | BF047 | +++ |
| BF012 | +++ | BF048 | +++ |
| BF014 | ++ | BF049 | +++ |
| BF017 | +++ | BF051 | +++ |
| BF018 | +++ | BF052 | +++ |
| BF019 | +++ | BF053 | +++ |
| BF020 | ++ | BF054 | +++ |
| BF021 | ++ | BF056 | ++ |
| BF022 | ++ | BF058 | +++ |
| BF023 | ++ | BF059 | ++++ |
| BF024 | ++ | BF060 | +++ |
| BF025 | ++ | BF062 | +++ |
| BF026 | ++ | BF064 | +++ |
| BF028 | ++ | BF066 | +++ |
| BF030 | ++++ | BF067 | ++++ |
| BF031 | ++ | BF070 | +++ |
| BF033 | ++++ | BF080 | ++++ |
| BF044 | ++ | BF081 | +++ |
| BF035 | +++ | BF082 | +++ |
| BF036 | +++ | BF084 | +++ |
| BF037 | ++++ | BF085 | +++ |
| BF077 | ++ | BF088 | +++ |
| BF078 | ++ | BF089 | ++++ |
| BF098 | ++++ | BF090 | ++++ |
| BF100 | ++++ | BF094 | +++ |
| BF101 | +++ | BF076 | ++ |
| BF104 | ++++ | BF114 | ++++ |
| BF106 | +++ | BF137 | ++++ |
| BF108 | +++ | BF072 | ++++ |
| BF111 | ++++ | BF139 | +++ |
| BF112 | ++++ | BF143 | +++ |
| BF113 | ++++ | BF075 | 0 |
| | | BF146 | 0 |

### Conclusion

Small molecule compounds of the invention bind sortilin.

### Example 16: Formation of Ternary Complex Facilitated by Bifunctional Compounds Wherein S_{L} is Derived from a Small Molecule

### Aim

To demonstrate the ability of bifunctional compounds to form ternary complexes with sortilin and a target molecule.

### Materials and Methods

In these experiments, bifunctional compounds were titrated in 5 μL total volume over 24-point in a 384-well TTP LVDS plate (SPT Labtech) to a final buffer composition of 50 mM Bis-Tris Propane pH 8, 50 mM NaCl, 0.05% Tween20 and 8% DMSO using a Mosquito LV pipetting robot (SPT Labtech).

To a black 384-well plate (Corning) the following were added: 5 μL 8 nM MAb Anti-6HIS Tb (Cisbio) in 50 mM Bis-Tris Propane pH 8, 50 mM NaCl, 0.05% Tween20, 5 μL 200 nM Streptavidin-d2 (Cisbio) in 50 mM Bis-Tris Propane pH 8, 50 mM NaCl, 0.05% Tween20, 5 μL 400 nM Sortilin-6His in 50 mM Bis-Tris Propane pH 8, 50 mM NaCl, 0.05% Tween20, Tween20 and 5 μL bifunctional compound in 50 mM Bis-Tris Propane pH 8, 50 mM NaCl, 0.05% Tween20, with or without 8% DMSO

After 2.5 hours incubation at room temperature the HTRF signal ratio 665/620 nm was determined on a ClarioStar plate reader (BMG Labtech). All reactions were run in triplicates.

FIG. 16 shows the resulting HTRF (665/620 nm) signal baseline corrected to the lowest local signal plotted against bifunctional compound concentration (5 μM-0.6 pM) after 2.5 hrs incubation. Binary interaction with sortilin was exhibited by the bifunctional compounds as visualized by bell shaped curves.

TABLE 5
AUC of ternary complex formation (AUC scores - AUC <
25.000: +; AUC > 25.000: ++)

| Compound | AUC score |
|---|---|
| BF017 | + |
| BF018 | ++ |
| BF019 | + |
| BF020 | ++ |
| BF021 | ++ |
| BF023 | + |
| BF025 | + |
| BF026 | ++ |

### Conclusion

Compounds of the invention mediate ternary complex formation between sortilin and warhead binding target.

### Example 17: Internalization of Target Extracellular Protein in Cells Mediated by Bifunctional Compounds

### Aim

Addressed in this example is the ability of sortilin to facilitate internalization of an extracellular target by a bifunctional compound where S_{L} is derived from a small molecule.

### Materials and Methods

In this example, the small molecule degraders consist of a sortilin binder linked to warhead biotin (BF017 to BF028) to target fluorescent NeutrAvidin-650 (NA650).

Cellular uptake of NA650 was investigated in HEK293 cells expressing full length sortilin receptor using a methodology analogous to example 3.

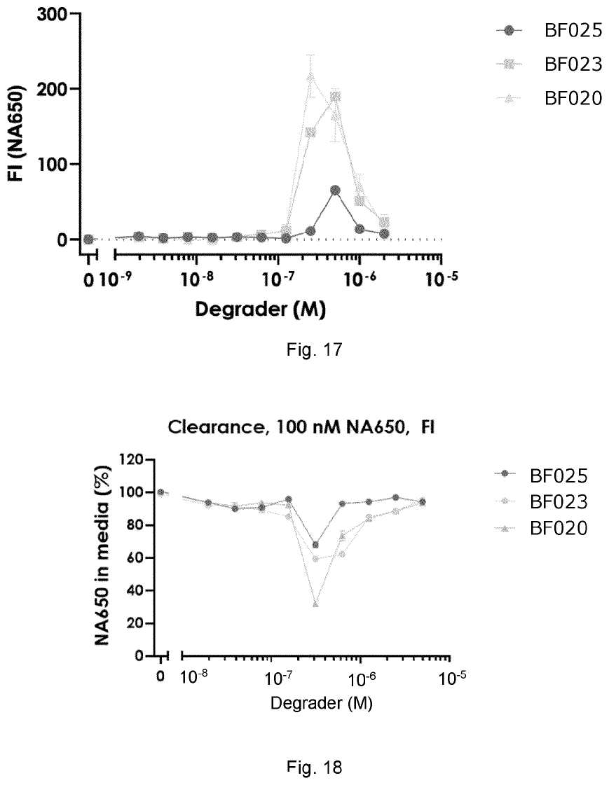

FIG. 17 shows cell associated FI signal following 3 h incubation of HEK293/sortilin with a concentration series (2 nM to 2 μM) of compounds B025, B023, or B020 together with NA650 (100 nM).

TABLE 6
AUC of target internalization (AUC scores - AUC <
100: +; AUC > 100: ++)

| Compound | AUC score |
|---|---|
| BF017 | ++ |
| BF018 | ++ |
| BF019 | + |
| BF020 | ++ |
| BF021 | ++ |
| BF022 | + |
| BF023 | + |
| BF024 | + |
| BF025 | + |
| BF026 | ++ |
| BF028 | ++ |

### Conclusion

Compounds of the invention mediate cellular internalisation of target.

### Example 18: Bifunctional Compounds Mediate Depletion of Target from Extracellular Cell Culture Medium

### Aim

Addressed in this example is the ability of a small molecule bifunctional compounds to deplete a target from the extracellular space as investigated by evaluation of fluorescent intensity (FI) in cell culture supernatant following co-incubation of cells with bifunctional compounds and NA650.

### Materials and Methods

HEK293/sortilin cells (35k cells/96-well) were seeded in culture media (as described in Example 3) and incubated for 24 h in cell incubator. Culture media were discarded, and the cells were washed with assay medium containing FluoroBright DMEM (Gibco) and supplements as described in Example 3, before incubation in assay medium containing 100 nM NA650 and small molecule bifunctional compounds (20 nM to 5 μM). After an incubation period of up to 72 h, the media were harvested and the presence of NA650 were evaluated by FI measurements of DyLight650 using a fluorescence plate reader (BMG Labtech Clariostar).

FIG. 18 shows NA650 FI signal in HEK293/sortilin cell culture supernatant following incubation with NA650 (100 nM) together with bifunctional compound BF025, BF023 or BF020 (20 nM to 5 μM) as indicated for 72 h.

### Conclusion

This example demonstrates that small molecule bifunctional compounds can induce depletion of target from cell culture supernatant.

### Example 19: Targets of Bifunctional Compounds are Sorted from the Extracellular Space to Lysosomes for Degradation

### Aim

This example investigates the ability of a small molecule bifunctional compounds to mediate degradation of a target in lysosomes. This was studied by evaluation of fluorescent target level in blotted cell lysates following inhibition of lysosomal function.

### Materials and Methods

Intracellular levels of NA650 following inhibition of lysosomal function, was investigated in HEK293/sortilin cells. Cells were seeded in 4-well plates (250k cells/well) in 250 μL culture medium as described in example 3 incubated overnight.

Culture medium was replaced with assay medium (as described in example 3) with or without: NA650 (100 nM) (Thermo Fischer), B025 (300 nM), sortilin binder AF38469 (10 μM) and leupeptin (80 μM) (Sigma Aldrich). Cells were incubated 24 h in cell incubator before they were washed and lysed in lysis buffer (as described in example 4). Lysates were centrifugated and the supernatants were mixed with LDS sample buffer (Thermo Fisher) and DTT (Sigma Aldrich) and boiled for 5 minutes before proteins were separated on a 4-12% Bis-Tris gel (Thermo Fisher). Proteins were blotted onto a nitrocellulose membrane (Thermo Fisher), and the DyLight650 signal was detected using an iBright 1500 (Thermo Fisher). Following evaluation of FI, membrane was blocked for 1 h in blocking buffer (TBS (Fisher Scientific) with 1% Tween20 (Sigma Aldrich) and 5% milk powder) and incubated with primary anti-β actin antibody (1:3500) (Sigma Aldrich) for 2 h at room temperature. The blot was washed 3 times in wash buffer (TBS (Fisher Scientific)+1% Tween20 (Sigma Aldrich)+0.5% milk powder) and incubated with secondary anti-mouse-HRP antibody (1:3500) (Abcam) for 1 h room temperature before it was washed 3× in wash buffer and the blot was developed and analyzed using ECL reagents (Cytiva) and iBright 1500 (Thermo Fisher).

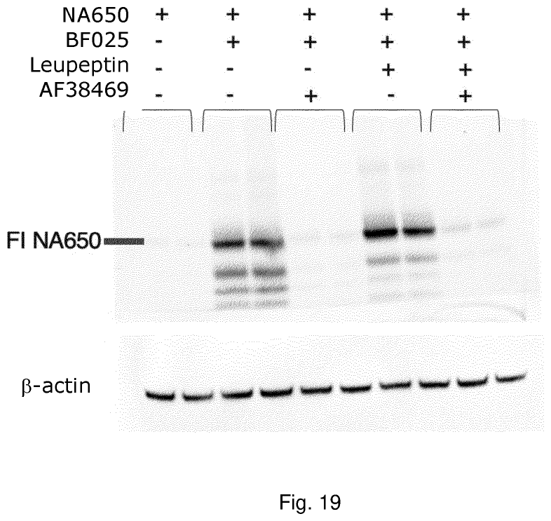

FIG. 19 shows FI in lysate of HEK293/sortilin harvested after 24 h incubation with NA650 (100 nM), B025 (300 nM), AF38469 (10 M) and leupeptin (80 μM) as indicated. A band corresponding to NA650 is visible in cells incubated with B025 alone, but absent when cells are co-incubated with competitive sortilin binder AF38469. In addition, the band increases in cells incubated with leupeptin. NA650 degradation products of lower molecular weight are most intense in cells where lysosomal activity is not inhibited with leupeptin. Anti-beta-actin Western blot is shown as control.

### Conclusion

This example provides data demonstrating intracellular accumulation of target upon inhibition of lysosomal cysteine protease activity by addition of leupeptin. This shows, that internalization mediated by small molecule bifunctional compounds results in lysosomal degradation of target. Furthermore, data shows that small molecule bifunctional compounds mediated internalization is potently inhibited by addition of a competitive sortilin binder, thus is sortilin facilitated.

### Example 20: Internalization of Extracellular Targets

### Aim

Addressed in this example is the ability of sortilin to mediate internalization of an extracellular target by bifunctional small molecules

### Materials and Methods

In this example, the small molecule degraders consist of a sortilin binder linked to warhead DNP to target anti-DNP antibodies (BF001 to BF016)

Cellular uptake of AlexaFluor488-anti-DNP antibody (Thermo Fisher) was investigated in HEK293 cells expressing full length sortilin receptor in a methodology analogous to example 3.

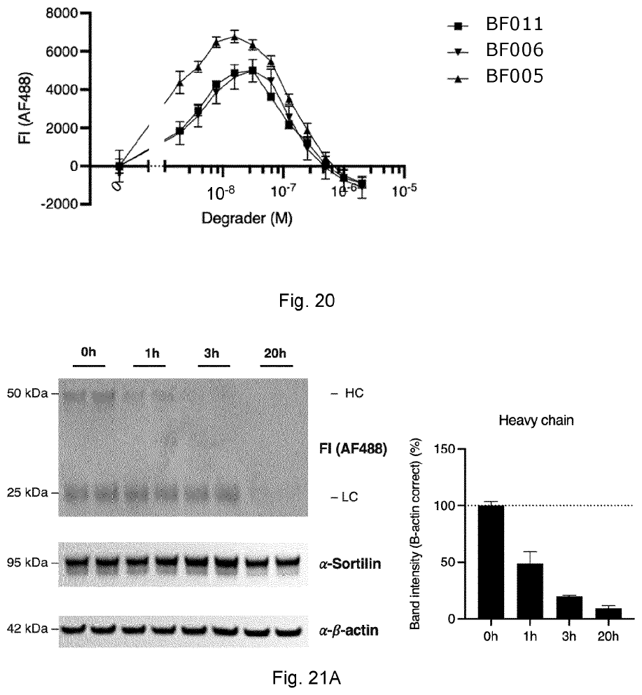

FIG. 20 shows cell associated FI signal following 3 h incubation of HEK293/sortilin with a concentration series (1.9 nM to 2 μM) of compounds BF011, BF006, or BF005 and AlexaFluor488-anti-DNP antibody (100 nM) (n=2). The bell-shaped response to increased bifunctional compound concentration is consistent with the hook effect observed in systems involving ternary complex formation.

TABLE 7
AUC of target internalization (AUC scores - AUC <
10.000: +; AUC > 10.000: ++)

| Compound | AUC score |
|---|---|
| BF004 | + |
| BF005 | ++ |
| BF006 | + |
| BF008 | + |
| BF010 | ++ |
| BF011 | + |
| BF014 | ++ |

### Conclusion

Compounds of the invention mediate cellular uptake of target.

### Example 21: Targets of Bifunctional Compounds are Sorted from the Extracellular Space to Lysosomes for Degradation

### Aim

Addressed in this example is the ability of a small molecule bifunctional compounds to mediate degradation of a target in lysosomes. This was investigated by evaluation of fluorescent target level in blotted cell lysates following inhibition of lysosomal function.

### Materials and Methods

Intracellular levels of AlexaFluor488-anti-DNP antibody (Thermo Fisher) following internalization was investigated in HEK293/sortilin cells. Cells (250k) were seeded in 4-well plates in 250 μL culture medium as described in Example 3 and incubated overnight.

Culture medium was replaced with assay medium (as described in Example 3) with AlexaFluor488-anti-DNP antibody (Thermo Fisher) (100 nM) and BF005 (30 nM). Selected samples were added sortilin inhibitor, AF38469 (10 μM) or a lysosomal protease inhibitor, Leupeptin (80 μM) (Sigma Aldrich). Cells were incubated 3-6: 30 h in cell incubator before they were washed with dPBS (Bionordika) and medium was either replaced with assay medium without BF005 and AlexaFluor488-anti-DNP antibody and incubated in cell incubator for up to 24 h or cells were lysed. Following incubation, cells were lysed in lysis buffer, and lysates were subjected to SDS-PAGE before transfer of proteins to a nitrocellulose membrane as described in Example 4. AlexaFluor488 FI signal was detected using an iBright 1500 (Thermo Fisher). Following evaluation of FI signal, membrane was blocked and incubated with primary anti-β-actin antibody (1:3500) (Sigma Aldrich) and secondary anti-mouse-HRP antibody (1:3500) (Abcam) and developed and analyzed using ECL reagents (Cytiva) and iBright 1500 (Thermo Fisher), as described in example 4. Subsequently, the membrane was stripped in Restore stripping buffer and probed with anti-human-NTR3 (sortilin) Ab (1:3500, BD Bioscience) and HRP conjugated anti-mouse-IgG (1:3500, Abcam.

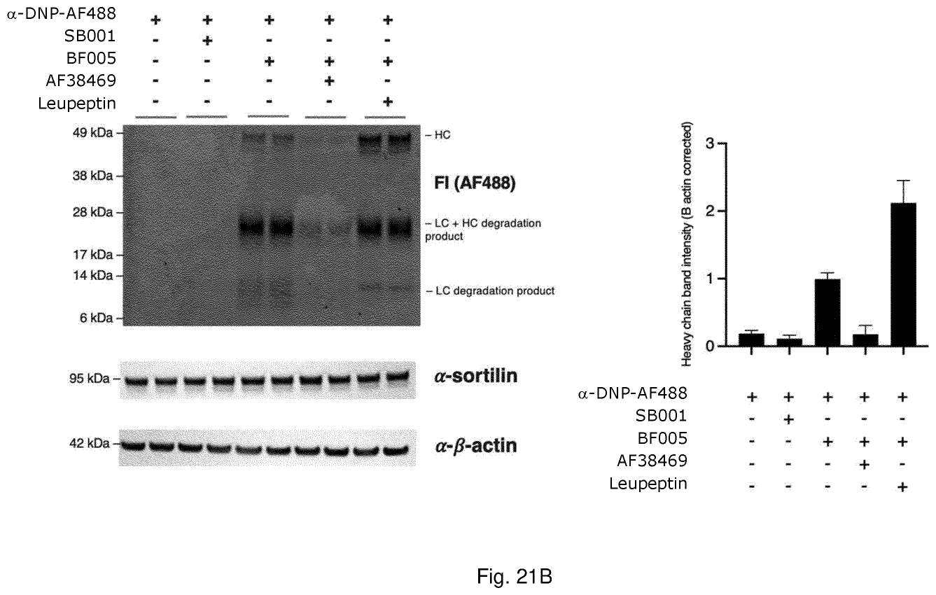

FIG. 21A shows FI in lysates of HEK293/sortilin incubated 3 h with AlexaFluor488-anti-DNP antibody (100 nM) and BF005 (30 nM) before removal of target and degrader by replacement of medium with assay medium further incubated for 0 h to 24 h before cells were lysed as described previously. A band corresponding to Ig heavy chain (HC) is visible at 0 h time point with band intensity decreasing with increasing incubation time. Anti-β-actin and anti-sortilin Western blots are shown as control. Bar graph shows quantification of FI signal of the HC band normalized to β-actin signal (mean value+/−SEM) (n=2).

In a different experiment, HEK293/sortilin cells were incubated with or without AlexaFluor488-anti-DNP antibody (100 nM), BF005 (30 nM), sortilin binder AF38469 (10 μM) and leupeptin (80 μM). Cells were incubated 6:30 h in cell incubator before they were washed with dPBS and lysed for evaluation of intracellularly accumulated target as described above.

FIG. 21B shows FI in lysates of HEK293/sortilin incubated 6: 30 h with AlexaFluor488-anti-DNP antibody (100 nM), BF005 (30 nM) and leupeptin (80 μM) as indicated. SB001 (30 nM) and AF38469 (10 μM) were included as control. A band corresponding to Ig heavy chain (HC) is visible in lysates from cells co-incubated with target and BF005 degrader. The intensity of the band is markedly increased upon inhibition of lysosomal degradation with leupeptin, and absent in cells where competitive sortilin binder (AF38469) or control degrader without DNP warhead (SB001) is added. Bar graph shows quantification of FI signal of the HC band normalized to β-actin signal (mean value+/−SEM) (n=2).

### Conclusion

This example provides data demonstrating a rapid degradation of internalized target. Further, data shows intracellular accumulation of target upon inhibition of lysosomal cysteine protease activity by addition of leupeptin, indicating lysosomal degradation of the desired target. In addition, data shows that small molecule bifunctional compounds mediated internalization is potently inhibited by competitive sortilin binding.

### Example 22: Compounds of the Invention Mediate Cellular Uptake of Target Proteins Via Sortilin Receptor

### Aim

This example investigates the role of sortilin in cellular internalization of an extracellular target mediated by bifunctional compounds of the invention.

### Materials and Methods

Cellular uptake of target was investigated in untransfected HEK239 cells and HEK293 cells expressing sortilin receptor in a methodology analogous to example 3.

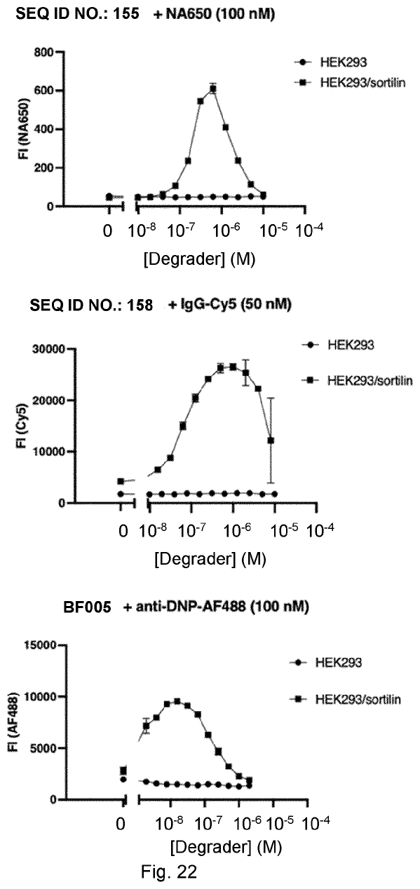

FIG. 22 shows cell associated FI signal following 3 h incubation of HEK293 or HEK293/sortilin with a concentration series (2 nM to 2 μM) of three different bifunctional compounds: SEQ ID NO.: 155, NB-link-RQLL (SEQ ID NO.: 158), or small molecule bifunctional compound (BF005) and corresponding targets as indicated. Data show a bell-shaped FI response to increasing degrader concentration in sortilin expressing cells across all bifunctional compound modalities. No response is observed in HEK293 cells.

### Conclusion

This example demonstrates that bifunctional compound mediated uptake of target requires cellular expression of sortilin.

### Example 23: Binding to TNFalpha

### Aim

To assess the binding to TNFa of compounds according to the present disclosure.

### Materials and Methods

MST was measure analogously to example 1 using TNFa-6his.

### Results

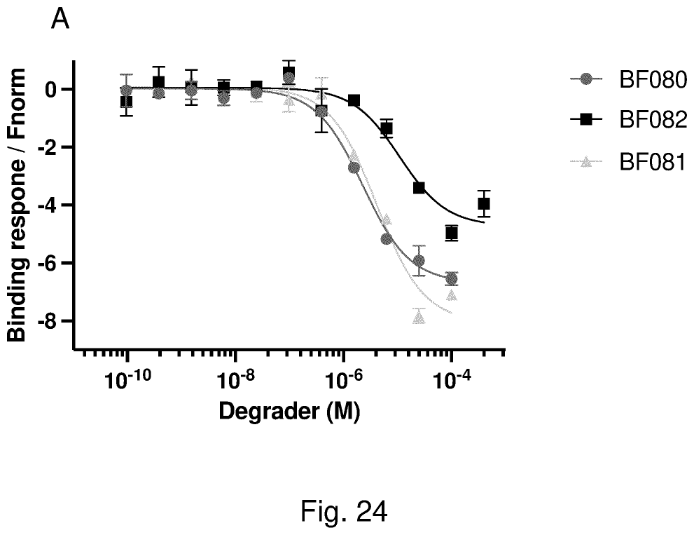

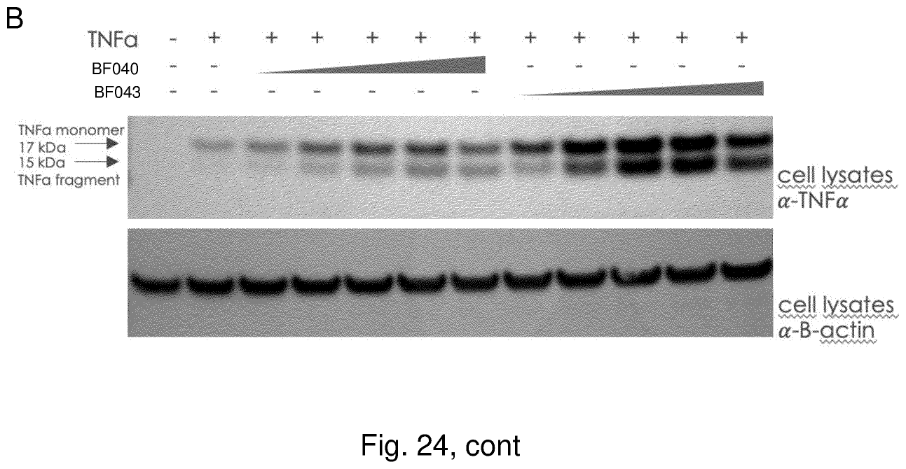

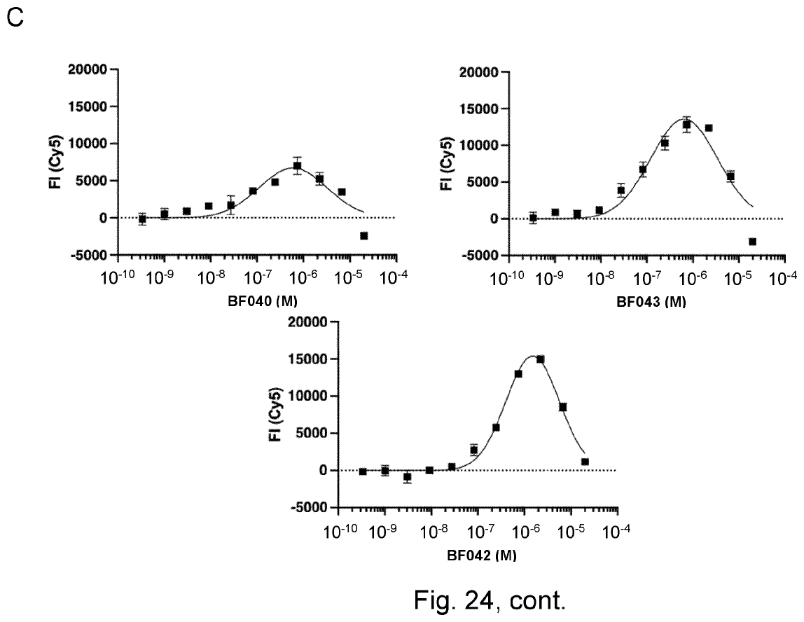

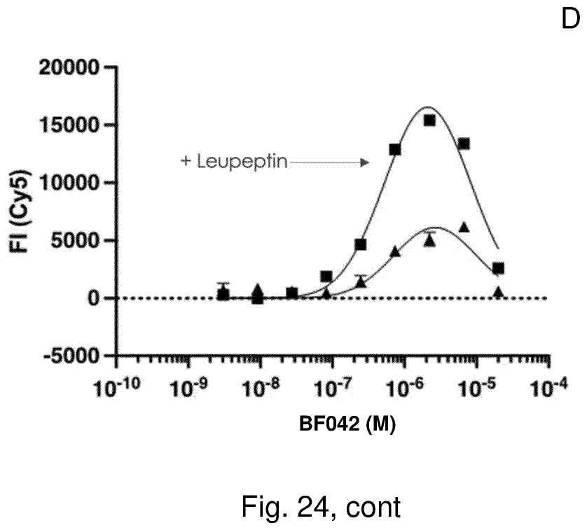

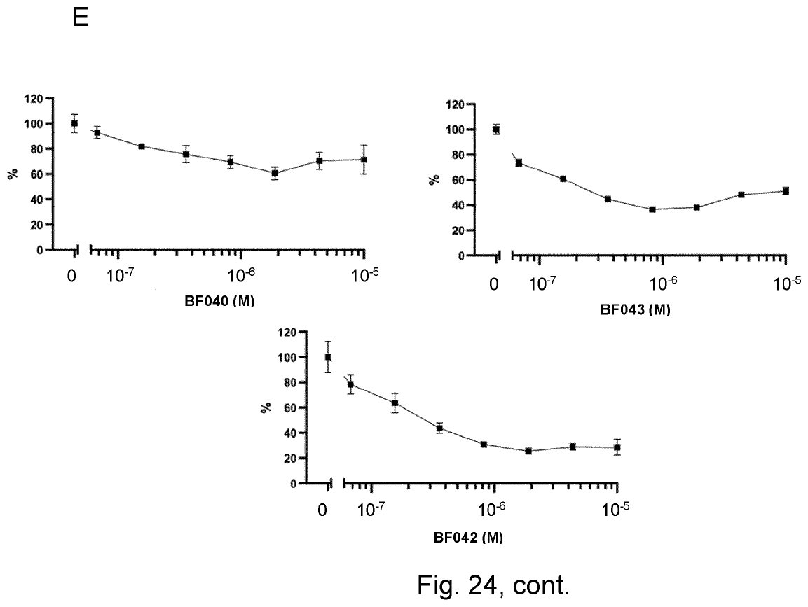

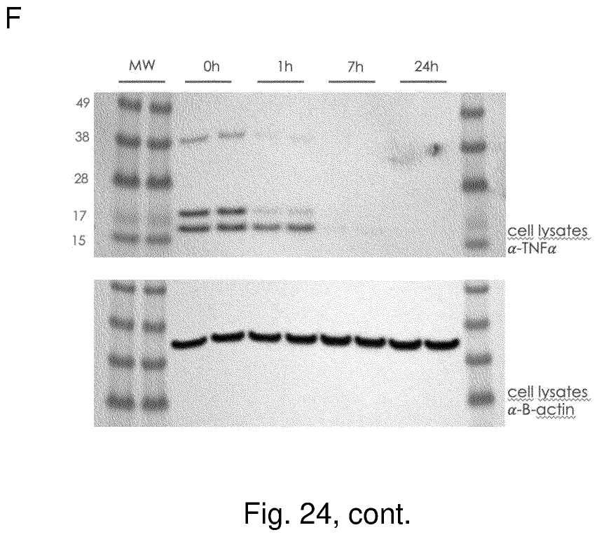

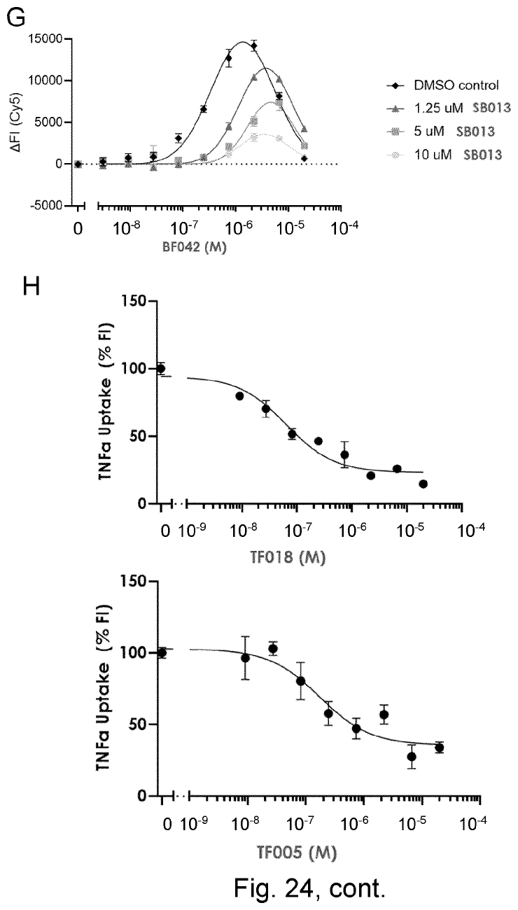

FIG. 24A Detection of binding by titrated BF080, BF081, and BF082 with constant NTA labeled TNFa-6His (100 nM) in MST analysis. (Error bars=S.D.; n=2)

TABLE 8
Kd of TNFalpha binding (Kd scores - K_{D} < 2 μM: ++;
K_{D} > 2 μM: +)

| Compound | K_{D} (score) | Compound | K_{D} (score) |
|---|---|---|---|
| BF040 | + | BF081 | ++ |
| BF080 | ++ | BF082 | + |
| BF079 | ++ | BF077 | ++ |
| BF076 | ++ | BF0100 | ++ |

### Example 24: Cellular Uptake of TNFalpha Mediated by Bifunctional Compounds

### Aim

To assess the binding of compounds according to the present disclosure to TNFalpha.

### Materials and Methods

Cellular uptake of TNFalpha was investigated in HEK293 cells expressing full length sortilin receptor using a methodology analogous to example 3 with TNFalpha target.

FIG. 24B shows mouse anti-TNFalpha (Invitrogen, MA5-23720) Western blot of cell lysates following 24 h incubation of HEK393/sortilin with a concentration series (100 nM-10 μM) of compounds BF040 and BF043 together with TNFalpha (100 nM). Control cells were incubated without addition of compounds and TNFalpha or with TNFalpha alone. Anti-β-actin (Sigma, A5441) Western blot is shown as control.

FIG. 24C shows cell associated FI signal following 24 h incubation of HEK293/sortilin with a concentration series (0.5 nM to 20 μM) of BF040, BF043 and BF042 and Cy5-TNFalpha (100 nM) in the presence of 80 μM leupeptin. Efficacy of individual bifunctional compounds was assessed as AUC of TNFalpha internalisation (Table 9)

TABLE 9
AUC of TNFalpha internalization (AUC scores -
AUC > 25.000: ++++; AUC 10.000-
25.000: +++; AUC 1000-10.000: ++; AUC < 1000: +)

| Compound | P2P AUC (score) | Compound | P2P AUC (score) |
|---|---|---|---|
| BF030 | + | BF081 | +++ |
| BF031 | ++ | BF082 | + |
| BF033 | ++ | BF083 | +++ |
| BF044 | ++ | BF084 | ++++ |
| BF035 | ++ | BF085 | +++ |
| BF036 | ++ | BF086 | ++++ |
| BF037 | ++ | BF088 | +++ |
| BF038 | ++ | BF090 | ++++ |
| BF039 | ++ | BF094 | ++++ |
| BF040 | +++ | BF076 | +++ |
| BF041 | ++ | BF077 | ++++ |
| BF042 | +++ | BF078 | ++ |
| BF043 | +++ | BF079 | + |
| BF044 | +++ | BF096 | ++++ |
| BF045 | ++ | BF098 | +++ |
| BF046 | ++ | BF099 | ++ |
| BF047 | ++ | BF100 | +++ |
| BF048 | ++ | BF101 | +++ |
| BF049 | ++ | BF102 | +++ |
| BF051 | + | BF104 | +++ |
| BF052 | + | BF106 | ++++ |
| BF053 | + | BF108 | +++ |
| BF054 | + | BF111 | ++++ |
| BF056 | + | BF112 | ++++ |
| BF058 | + | BF113 | +++ |
| BF059 | + | BF114 | +++ |
| BF060 | + | BF119 | ++++ |
| BF062 | + | BF121 | ++++ |
| BF064 | + | BF125 | ++++ |
| BF066 | + | BF126 | ++++ |
| BF067 | + | BF129 | ++++ |
| BF070 | + | BF050 | ++ |
| BF080 | + | BF131 | ++++ |
| BF063 | + | BF133 | +++++ |
| BF072 | + | BF137 | + |
| BF139 | + | BF146 | + |
| BF143 | + | BF147 | ++++ |
| BF074 | + | BF148 | ++++ |
| BF075 | + | BF149 | ++++ |
| BF145 | + | | |

FIG. 24D shows cell associated FI signal following 24 h incubation of HEK293/sortilin cells with BF042 (0.5 nM to 20 μM) and Cy5-TNFalpha with or without addition of leupeptin (80 μM). Inhibition of lysosomal degradation by addition of leupeptin results in a marked increases cell associated signal.

In different experiments, HEK293/sortilin cells were incubated with compound gradient series and 20 nM TNFalpha for up to 72 h. Following incubation, remaining TNFalpha in conditioned media was evaluated using human TNF-alpha Quantikine ELISA Kit (R&D systems, DTA00D).

FIG. 24E shows TNFalpha levels in conditioned media of HEK293/sortilin cells 72 h after addition of 20 nM TNFalpha and bifunctional molecule BF040, BF043 or BF042. TNFalpha is shown in % normalised to level measured in conditioned media from cells without addition of bifunctional molecule. Maximum clearance rates (calculated as added TNFalpha (100%) subtracted remaining TNFalpha (%)) after 72 h were 39.4% for BF040, 63.3% for BF043 and 74.4% BF042.

To monitor cellular degradation of target over time, a pulse-chase experiment was performed: HEK293/sortilin cells were pre-incubated with bifunctional compound (3 μM) and TNFalpha (100 nM) for 24 h before replacing culture supernatant with fresh media without target and bifunctional compound. Following replacement of media, cells were incubated for up to 24 h and TNFa levels was assessed at different timepoints by Western blotting of cell lysates.

FIG. 24F shows anti-TNFalpha Western blotting of HEK293/sortilin cell lysates harvested at indicated timepoints (0 h to 24 h) following 24 h pre-incubation with BF042 (3 μM) and TNFalpha (100 nM). Pre-incubation media was replaced at timepoint 0 h. Anti-beta-actin Western blotting is shown as control. The cell lysate bands representing TNFalpha are most intense at timepoint 0 h, markedly decreased at 1 h and barely detectable after 7 h incubation indicating rapid degradation of TNFalpha after internalisation.

FIG. 24G shows cell associated FI signal following 24 h incubation of HEK293/sortilin with a concentration series (0.5 nM to 20 μM) of BF042 and Cy5-TNFalpha (100 nM) with or without addition of sortilin binder SB013 (1.25, 5.0 and 10 μM) or DMSO . . .

FIG. 24H shows cell associated FI signal following 24 h incubation of HEK293/sortilin with or BF077 (1 μM) and Cy5-TNFalpha (100 nM) in the presence of increasing concentrations of TF018 or TF005 as competitor of TNFalpha binding.

Conclusion Compounds of the invention mediate cellular internalisation and lysosomal degradation of TNFalpha.

### Example 25: In Vivo Efficacy in Mouse Model of Acute Systemic Inflammation

### Aim

To assess the ability to remove TNFalpha from plasma in an animal model of systemic inflammation.

### Materials and Methods

Degrader in vivo efficacy was investigated in a mouse model of lipopolysaccaride (LPS) induced acute systemic inflammation. Female C57BL/6J mice, 8 weeks of age were used in this model, and LPS was dosed as IP injection at a final concentration of 0.5 mg/kg in PBS at 10 ml/kg. 30 min to 3 hours prior LPS administration, animals were dosed with test compound (100-0.3 mg/kg/IP), dexamethasone (Dex) control (0.5 mg/kg/PO) or vehicle control (IP). Untreated (normal) animals were included as control for LPS induced

TNFa expression. One hour post LPS administration, blood samples were collected and TNFa level was assessed by Cytometric Bead Array assay.

### Results

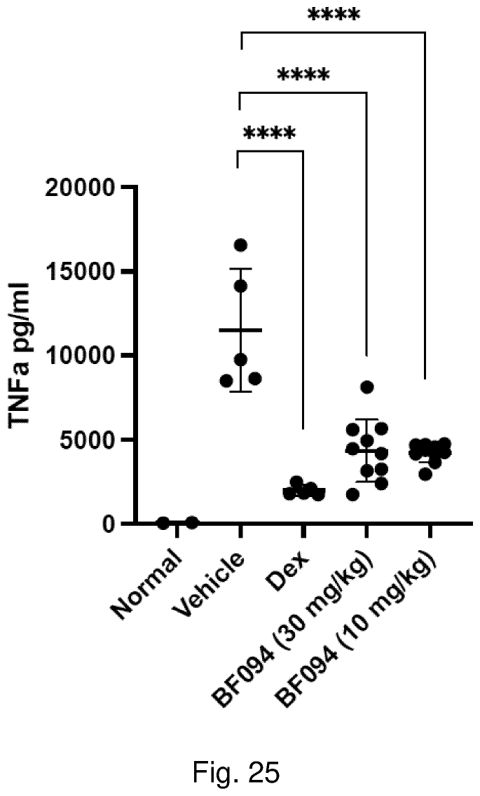

FIG. 25 shows TNFa plasma levels in animals treated with vehicle, Dex (0.5 mg/kg), and BF094 (10 or 30 mg/kg) 1 hour after administration of LPS. Normal animals were untreated controls. n=5 (normal/vehicle/Dex) or n=10 per group. A significant decrease in plasma TNFa is observed in animals after treatment with BF094 compared to vehicle group. n=5 (normal/vehicle/Dex) or n=10 animals per group. **** p<0.0001 (one-way ANOVA).

### Conclusion

The compounds according to the present invention can mediate targeted protein degradation in vivo. The bifunctional compounds can remove the target protein of interest from the plasma of animals. Specifically, the results show that TNFa bifunctional compounds reduce TNFalpha levels in the plasma of LPS stimulated animals. The reduction in TNFalpha is comparable to the effect produced by dexamethasone, a known anti-inflammatory agent.

### Example S1: Synthetic Protocols

### General Conditions

The compounds according to Formula (I) can be prepared from readily available starting materials by several synthetic approaches, using both solution-phase and solid-phase chemistry protocols or mixed solution and solid phase protocols. Examples of synthetic pathways are described below in the examples. All reported yields are non optimized yields. Unless otherwise stated, compounds of Formula (I) and related formulae obtained as a racemic mixture can be separated to provide an enantiomerically enriched mixture or a pure enantiomer.

The standard conditions are as follow:

All temperatures are in degrees Celsius (° C.) and are uncorrected.

Reagent grade chemicals and anhydrous solvent were purchased from commercial sources and unless otherwise mentioned, were used without further purification.

Silica gel chromatography was performed on Biotage instruments using pre-packaged disposable SiO2 stationary phase columns with eluent flow rate range of 15 to 200 mL/min, UV detection (254 and 280 nm).

Reverse phase preparative HPLC was carried out using C18 columns, UV detection (215, 220 and 254 nm) eluting with gradients of MeCN in water (10 mM NH4HCO3) or MeCN in water (0.04% HCl) or MeCN in water (0.2% HCOOH).

The analytical HPLC chromatograms were performed mainly using two methods:
using an Shimadzu 20AB (The gradient was 10-80% B in 3.00 min with a hold at 80% B for 1.0 min, 80-10% B in 0.01 min, and then held at 10% for 0.50 min (0.01-4.00 min: 0.5 ml/min flow rate; 4.01-4.50 min: 1.0 ml/min flow rate). Mobile phase A was 0.037% Trifluoroacetic Acid in water, mobile phase B was 0.018% Trifluoroacetic Acid in acetonitrile. The column used for chromatography was a Kinetex 5 um C18 100A 50*2.1 mm. Detection methods are diode array (DAD)
using Shimadzu 20AD (The gradient was 10-80% B in 3.00 min with a hold at 80% B for 0.9 min, 80-10% B in 0.03 min, and then held at 10% for 0.57 min (0.01-3.91 min: 0.8 ml/min flow rate; 3.92-4.50 min: 1.2 ml/min flow rate). Mobile phase A was 10 mM NH4HCO3 in water, mobile phase B was 100% acetonitrile. The column used for chromatography was a X-bridge Shield RP18 2.1*50 mm column (5 um particles). Detection methods are diode array (DAD).

Products were analyzed by LCMS mainly by two methods:
on Agilent 1200 & 6110B using (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) as well as positive electrospray ionization. MS range was 100-1000.
on Agilent 1200 & 6110B using (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H2O+10 mM NH4HCO3, mobile phase B was Acetonitrile. The column used for chromatography was a Xbridge Shield RP18 2.1*50 mm column (5 um particles). Detection methods are diode array (DAD) as well as positive electrospray ionization. MS range was 100-1000.

Intermediates were analyzed by LCMS mainly using two methods:
on Shimadzu LC-20AD&MS 2020 The column used for chromatography was a Luna-C18 2.0*30 mm, (3 um particles). Detection methods are diode array (DAD). MS mode was positive electrospray ionization. MS range was 100-1000. Mobile phase A was 0.037% Trifluoroacetic acid in water, and mobile phase B was 0.018% Trifluoroacetic acid in HPLC grade acetonitrile. The gradient was 10-80% B in 4.30 min. 10% B in 0.01 min, 10-80% B (0.01-3.50 min), 80-10% B (3.50-3.80 min), with a hold at 10% B for 0.50 min. The flow rate was 0.8 mL/min (0.01-3.80 min) and 1.2 mL/min (3.81-4.30 min).
on Agilent 1200 & 6110B using Agilent 1200 & 1956A column used for chromatography was Xbridge Shield RP18 2.1*50 mm, (5 um particles). Detection methods are diode array (DAD). MS mode was positive electrospray ionization. MS range was 100-1000. Mobile phase A was 10 mM Ammonium bicarbonate in water, and mobile phase B was HPLC grade acetonitrile. The gradient was 10-80% B in 3.00 min. 10% B in 0.00 min, 10-80% B (0.00-2.00 min) with a hold at 80% B for 0.48 min, 80-10% B (2.48-2.50 min) with a hold at 10% B for 0.5 min. The flow rate was 1.0 mL/min (0.00-2.48 min) and 1.2 mL/min (2.50-3.00 min).

The 1H NMR spectra were recorded from 11 instruments, the data were different, and shown as below:

NMR-A: Bruker AVANCE NEO 400 MHz/54 mm instrument (MRCA 400/54/ASC, 16971)

NMR-B: Bruker AVANCE III 400 MHz/54 mm UltraShield Plus, long hold time, instrument (BZH 439′400′701, D335/54-6776)

NMR-C: Bruker AVANCE III 400 MHZ/54 mm Ascend instrument (BZH 994′400′701, D315′54-9223)

NMR-D: Varian 400MR 400 MHz/54 mm instrument (MRCA 400/54/ASC, MRYOO20874)

NMR-E: Bruker AVANCE III 400 MHz/54 mm Ascend instrument (BZH 993′400′701, D315′54-9213)

NMR-F: Bruker AVANCE III 400 MHz/54 mm Ascend instrument (BZH 1157′400′701, D315′54-9574)

NMR-H: Bruker AVANCE III 400 MHz/54 mm Ascend instrument (BZH 1126′400′701, D315′54-9527)

NMR-J: Bruker AVANCE NEO 400 MHZ/54 mm Ascend instrument (BZH 1396′400′701, D315′54-10089)

NMR-K: Bruker AVANCE NEO 400 MHZ/54 mm Ascend instrument (BZH 1373′400′701, D315′54-10026)

NMR-S: Varian 400MR 400 MHz/54 mm instrument (MRCA 400/54/ASC, 20609) NMR-Y: Varian 400MR 400 MHz/54 mm instrument (MRCA 400/54/ASC, 20188) The chemical shifts are referenced to solvent peaks, which in 1H NMR appear at 7.27 ppm for CDCl3, 2.50 for DMSO-d6, 4.79 for D20 and 3.31 ppm for CD3OD.

### SB001

### (2S)-5,5-dimethyl-2-[[6-[[5-(methylcarbamoyl)-1-naphthyl]oxy]pyridine-3-carbonyl]amino]hexanoic acid

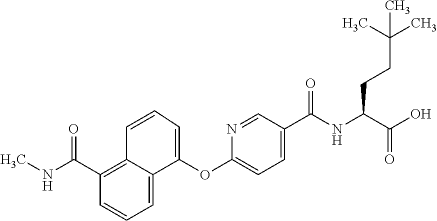 [SMILES: CNC(=O)c1cccc2c(Oc3ccc(C(=O)N[C@@H](CCC(C)(C)C)C(=O)O)cn3)cccc12]

### Step 1:5-hydroxy-N-methyl-naphthalene-1-carboxamide

To a solution of methanamine (2.67 g, 39.56 mmol, 8 eq, HCl) in DCE (40 mL) was added dropwise trimethylaluminium (2 M, 24.73 mL, 10 eq) at 0° C. After addition, the mixture was stirred at this temperature for 15 min, and then methyl-5-hydroxynaphthalene-1-carboxylate (1 g, 4.95 mmol, 1 eq) in DCE (10 mL) was added dropwise at 0° C. The resulting mixture was stirred at 80° C. for 16 hours. The reaction mixture was then cooled to 0° C., DCM (100 mL) was added followed by a 1N HCl solution until solubilization of aluminium salts (100 mL). The mixture was extracted with DCM (100 mL×3). The combined organic layers were dried with Na₂SO₄, filtered and concentrated in vacuo to give 5-hydroxy-N-methyl-naphthalene-1-carboxamide (500 mg, 50.24% yield, 100% purity) as a white solid.

### Step 2: Methyl 6-[[5-(methylcarbamoyl)-1-naphthyl]oxy]pyridine-3-carboxylate

A mixture of methyl 6-bromopyridine-3-carboxylate (697.35 mg, 3.23 mmol, 1.2 eq), 5-hydroxy-N-methyl-naphthalene-1-carboxamide (541 mg, 2.69 mmol, 1 eq), potassium phosphate (570.70 mg, 2.69 mmol, 1 eq) and CuI (512.04 mg, 2.69 mmol, 1 eq) in DMSO (10 mL) was degassed and purged with N₂ three times, and then the mixture was stirred at 120° C. for 16 hr under a N₂ atmosphere. The mixture was washed with H₂O (40 mL) and extracted with EA (20 mL×4). The organic layer was dried with Na₂SO₄, filtered and concentrated under reduce pressure.

The residue was purified by flash silica gel chromatography (ISCO®; 12 g SepaFlash® Silica Flash Column, Eluent of 0˜60% Ethyl acetate/Petroleum ether gradient at 25 mL/min). Methyl 6-[[5-(methylcarbamoyl)-1-naphthyl]oxy]pyridine-3-carboxylate (580 mg, 64.11% yield, 100% purity) was obtained as a white solid.

### Step 3: 6-[[5-(methylcarbamoyl)-1-naphthyl]oxy]pyridine-3-carboxylic acid

To a solution of methyl 6-[[5-(methylcarbamoyl)-1-naphthyl]oxy]pyridine-3-carboxylate (250 mg, 743.29 μmol, 1 eq) in a mixture of THF (15 mL) and H₂O (5 mL) was added LiOH·H₂O (62.38 mg, 1.49 mmol, 2 eq). The mixture was stirred at 25° C. for 2 hr. The mixture was concentrated under reduce pressure and the residue was acidified with HCl (1M) to pH 2. The residue was extracted with EA (20 mL×5). The combined organic layers were dried with Na₂SO₄, filtered and concentrated in vacuo. 6-[[5-(methylcarbamoyl)-1-naphthyl]oxy]pyridine-3-carboxylic acid (200 mg, 83.48% yield) was obtained as a white solid.

### Step 4:6-[[5-(methylcarbamoyl)-1-naphthyl]oxy]pyridine-3-carbonyl chloride

A mixture of 6-[[5-(methylcarbamoyl)-1-naphthyl]oxy]pyridine-3-carboxylic acid (200 mg, 620.51 μmol, 1 eq), oxalyl dichloride (157.52 mg, 1.24 mmol, 108.63 μL, 2 eq), DMF (4.54 mg, 62.05 μmol, 4.77 μL, 0.1 eq) in DCM (20 mL) was degassed at 0° C., and then the mixture was stirred at 25° C. for 5 hr under a N₂ atmosphere. The reaction mixture was concentrated under reduced pressure to give 6-[[5-(methylcarbamoyl)-1-naphthyl]oxy]pyridine-3-carbonyl chloride (211 mg, crude) as colourless gum.

### Step 5: (2S)-5,5-dimethyl-2-[[6-[[5-(methylcarbamoyl)-1-naphthyl]oxy]pyridine-3-carbonyl]amino]hexanoic acid

To a mixture of (2S)-2-amino-5,5-dimethyl-hexanoic acid (98.59 mg, 619.20 μmol, 1 eq) and TEA (93.98 mg, 928.81 μmol, 129.28 μL, 1.5 eq) in DCM (5 mL) was added 6-[[5-(methylcarbamoyl)-1-naphthyl]oxy]pyridine-3-carbonyl chloride (211 mg, 619.20 μmol, 1 eq) in DCM (5 mL). The mixture was stirred at 20° C. for 16 hr. The mixture was filtered and concentrated in vacuo. The residue was purified by prep-HPLC (HCl condition). (2S)-5,5-dimethyl-2-[[6-[[5-(methylcarbamoyl)-1-naphthyl]oxy]pyridine-3-carbonyl]amino]hexanoic acid (13.7 mg, 4.77% yield, 99.0% purity) was obtained as a white solid.

LCMS (ESI) [M+H]⁺ m/z: calc'd 464.21, found 464.1

¹H NMR (400 MHZ, DMSO-d₆) δ: ppm 0.85 (s, 10H) 1.14-1.36 (m, 2H) 1.63-1.87 (m, 2H) 2.87 (d, J=4.52 Hz, 3H) 4.21-4.38 (m, 1H) 7.27 (d, J=8.53 Hz, 1H) 7.38 (d, J=7.28 Hz, 1H) 7.52 (dd, J=8.41, 7.15 Hz, 1H) 7.58-7.67 (m, 2H) 7.88 (d, J=8.53 Hz, 1H) 8.10 (d, J=8.53 Hz, 1H) 8.33 (dd, J=8.78, 2.51 Hz, 1H) 8.45-8.58 (m, 2H) 8.64-8.74 (m, 1H)

### SB002

### 2-[[6-methyl-4-[4-(methylcarbamoyl)phenyl]-2-pyridyl]carbamoyl]-5-(trifluoromethyl)benzoic acid

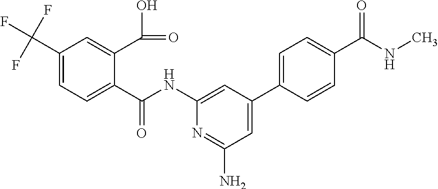 [SMILES: CNC(=O)c1ccc(-c2cc(N)nc(NC(=O)c3ccc(C(F)(F)F)cc3C(=O)O)c2)cc1]

### Step 1:4-(2-amino-6-methyl-4-pyridyl)-N-methyl-benzamide

To a mixture of 4-bromo-6-methyl-pyridin-2-amine (1 g, 5.35 mmol, 1 eq) and [4-(methylcarbamoyl)phenyl]boronic acid (1.05 g, 5.88 mmol, 1.1 eq) in dioxane/H₂O (18 mL) was added Na₂CO₃ (1.70 g, 16.04 mmol, 3.0 eq) and Pd(dppf)Cl₂ (391.21 mg, 534.65 μmol, 0.1 eq) in one portion at 25° C. under N₂. The mixture was heated to 90° C. and stirred for 4 hours. The reaction was filtered and the filtrate was concentrated. The residue was purified by silica gel chromatography eluted with (DCM:MeOH=91:9 to 90:10) to give a brown solid (100 mg, 7.75% yield, 100% purity).

### Step 2:4-[2-[1,3-dioxo-5-(trifluoromethyl) isoindolin-2-yl]-6-methyl-4-pyridyl]-N-methyl-benzamide

To a mixture of 5-(trifluoromethyl) isobenzofuran-1,3-dione (89.57 mg, 414.44 μmol, 1 eq) and 4-(2-amino-6-methyl-4-pyridyl)-N-methyl-benzamide (100 mg, 414.44 μmol, 1 eq) in AcOH (3 mL) was added at 25° C. under N₂. The mixture was heated to 120° C. and stirred at 120° C. for 2 hours, then concentrated in vacuo.

The residue was purified by column chromatography (SiO₂, PE:EtOAc=2:1 to 1:1) and concentrated. 4-[2-[1,3-dioxo-5-(trifluoromethyl) isoindolin-2-yl]-6-methyl-4-pyridyl]-N-methyl-benzamide (120 mg, 273.11 μmol, 65.90% yield, 100% purity) as a yellow oil was obtained.

### Step 3: 2-[[6-methyl-4-[4-(methylcarbamoyl)phenyl]-2-pyridyl]carbamoyl]-5-(trifluoromethyl)benzoic acid

To a mixture of 4-[2-[1,3-dioxo-5-(trifluoromethyl) isoindolin-2-yl]-6-methyl-4-pyridyl]-N-methyl-benzamide (120 mg, 273.11 μmol, 1 eq) in THF/water (3 mL) was added LiOH (19.62 mg, 819.33 μmol, 3 eq) in one portion at 25° C. under N₂ and stirred at 25° C. for 5 h. The mixture was acidified with 2M HCl to pH 2. DMF (0.5 mL) was added to help dissolve the product. The residue was purified by prep-HPLC (HCl condition, column: Boston Green ODS 150*30 mm*5 um; mobile phase: [water (0.05% HCl)-ACN]; B %: 18%-58%, 9 min) twice. After purification by prep-HPLC, and lyophilization, (2-[[6-methyl-4-[4-(methylcarbamoyl)phenyl]-2-pyridyl]carbamoyl]-5-(trifluoromethyl)benzoic acid (18.6 mg, 14.52% yield, 97.5% purity) as a white solid was obtained.

LCMS (ESI) [M+H]⁺ m/z: calc'd 458.12, found 458.0

¹H NMR (400 MHZ, DMSO-d6) δ: ppm 2.50 (br s, 3H) 2.81 (d, J=4.40 Hz, 3H) 7.44 (s, 1H) 7.76-7.87 (m, 3H) 7.97-8.06 (m, 3H) 8.14 (s, 1H) 8.33 (br s, 1H) 8.58 (br d, J=4.52 Hz, 1H) 11.17 (s, 1H)

### SB003

### (2S)-5,5-dimethyl-2-[[6-[[6-(methylcarbamoyl)-1-naphthyl]oxy]pyridine-3-carbonyl]amino]hexanoic acid

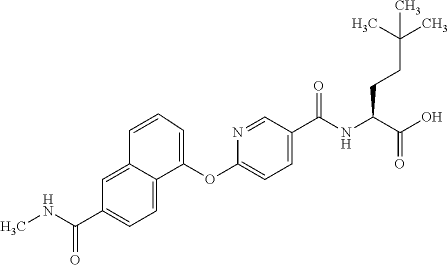 [SMILES: CNC(=O)c1ccc2c(Oc3ccc(C(=O)N[C@@H](CCC(C)(C)C)C(=O)O)cn3)cccc2c1]

### Step 1:5-hydroxynaphthalene-2-carboxylic acid

To a solution of 5-bromonaphthalene-2-carboxylic acid (5 g, 19.91 mmol, 1 eq) in dioxane (50 mL) was added KOH (4.47 g, 79.66 mmol, 4 eq) in H₂O (50 mL). Then tBuXphosPdG3 (395.94 mg, 497.86 μmol, 0.025 eq) was added under N₂, and the reaction was stirred at 110° C. under N₂ for 16 hr. LCMS showed the reaction was completed. The reaction mixture was acidified with 1M HCl until pH 4˜5 and filtered. To the filtrate was added water (50 mL), then extracted with EtOAc (2×50 mL). The combined organic layers were washed with brine (20 mL), dried over anhydrous Na₂SO₄, filtered and concentrated in vacuo. The residue was purified by silica gel chromatography eluted with (PE:EtOAc=100:1 to 1:1). 5-hydroxynaphthalene-2-carboxylic acid (3.2 g, 85.39% yield, 100% purity) was obtained as yellow solid.

¹H NMR (400 MHZ, DMSO-d₆) δ: ppm 13.01 (1H, br s) 10.32 (1H, br s) 8.49 (1H, d, J=1.5 Hz) 8.20 (1H, d, J=8.8 Hz) 7.90 (1H, dd, J=8.8, 1.6 Hz) 7.27-7.61 (2H, m) 7.00 (1H, dd, J=7.5, 0.8 Hz)

### Step 2:5-Hydroxy-N-methyl-naphthalene-2-carboxamide

5-hydroxynaphthalene-2-carboxylic acid (2.35 g, 12.49 mmol, 1 eq) in DCM (30 mL) was added DIEA (4.84 g, 37.46 mmol, 6.53 mL, 3 eq) EDCI (2.87 g, 14.99 mmol, 1.2 eq), HOBt (2.02 g, 14.99 mmol, 1.20 eq) and methanamine (843.17 mg, 12.49 mmol, 1 eq, HCl) at 20° C. under N₂. The reaction was stirred at 20° C. for 3 hrs. The reaction mixture was poured into water (100 mL). The aqueous layer was extracted with DCM (3×50 mL). The combined organic layers were washed with brine (50 mL), dried over anhydrous Na₂SO₄, filtered and concentrated in vacuo. The residue was purified by silica gel chromatography eluted with (PE:EtOAc=20:1 to 1:3).

5-hydroxy-N-methyl-naphthalene-2-carboxamide (2.4 g, 95.51% yield, 100% purity) as a red oil was obtained.

LCMS (ESI) [M+H]⁺ m/z: calc'd 202.0, found 202.2

¹H NMR (400 MHZ, CHLOROFORM-d) o: ppm 8.22-8.28 (2H, m) 7.80 (1H, dd, J=8.8, 1.5 Hz) 7.51 (1H, d, J=8.3 Hz) 7.37 (1H, t, J=7.9 Hz) 6.92 (1H, d, J=7.3 Hz) 6.31 (1H, br s) 3.10 (3H, d, J=5.0 Hz)

### Step 3: Methyl 6-[[6-(methylcarbamoyl)-1-naphthyl]oxy]pyridine-3-carboxylate

A mixture of 5-hydroxy-N-methyl-naphthalene-2-carboxamide (2.4 g, 11.93 mmol, 1 eq)methyl 6-bromopyridine-3-carboxylate (3.09 g, 14.31 mmol, 1.2 eq), copper (I) iodide (227.15 mg, 1.19 mmol, 0.1 eq) and potassium phosphate (5.06 g, 23.85 mmol, 2 eq) in DMSO (20 mL) was degassed and purged with N₂ three times, and then the mixture was stirred at 120° C. for 16 hr under an N₂ atmosphere. The reaction mixture was poured into water (50 mL), extracted with EA (3×30 mL). The combined organic layers were washed with brine (30 mL), dried over anhydrous Na₂SO₄, filtered and concentrated in vacuo. The residue was purified by silica gel chromatography eluted with (PE:EtOAc=100:1 to 1:2) to give methyl 6-[[6-(methylcarbamoyl)-1-naphthyl]oxy]pyridine-3-carboxylate (1.4 g, 33.96% yield, 97.3% purity) as a yellow solid.

LCMS (ESI) [M+H]⁺ m/z: calc'd 336, found 337

¹H NMR (400 MHZ, CHLOROFORM-d) δ: 8.70 (1H, d, J=1.9 Hz) 8.21-8.33 (2H, m) 7.87 (1H, d, J=8.8 Hz) 7.78 (1H, d, J=8.3 Hz) 7.71 (1H, dd, J=8.8, 1.6 Hz) 7.51 (1H, t, J=7.9 Hz) 7.29 (1H, dd, J=7.6, 0.8 Hz) 6.98 (1H, d, J=8.6 Hz) 6.21 (1H, br s) 3.84 (3H, s) 3.00 (3H, d, J=4.8 Hz)

### Step 4: 6-[[6-(methylcarbamoyl)-1-naphthyl]oxy]pyridine-3-carboxylic acid

To a solution of methyl 6-[[6-(methylcarbamoyl)-1-naphthyl]oxy]pyridine-3-carboxylate (1.57 g, 4.67 mmol, 1 eq) in a mixture of THF (45 mL) and H₂O (15 mL) was added LiOH·H₂O (391.76 mg, 9.34 mmol, 2 eq). The mixture was stirred at 25° C. for 2 hr. The mixture was concentrated under reduce pressure. The residue was acidified with HCl (1M) to pH 2. The residue was extracted with EA (20 mL×5). The combined organic layer were dried with Na₂SO₄, filtered and concentrated in vacuo. 6-[[6-(methylcarbamoyl)-1-naphthyl]oxy]pyridine-3-carboxylic acid (1.3 g, 82.09% yield, 95% purity) was obtained as a white solid.

LCMS (ESI) [M+H]⁺ m/z: calc'd 323.10, found 323.0

¹H NMR (400 MHz, METHANOL-d₄) δ: ppm 8.66 (1H, d, J=2.26 Hz) 8.44 (1H, d, J=1.51 Hz) 8.39 (1H, dd, J=8.66, 2.38 Hz) 7.90-7.96 (2H, m) 7.83-7.88 (1H, m) 7.62 (1H, t, J=7.91 Hz) 7.39 (1H, d, J=6.78 Hz) 7.16 (1H, d, J=8.53 Hz) 2.97 (3H, s)

### Step 5: 6-[[6-(Methylcarbamoyl)-1-naphthyl]oxy]pyridine-3-carbonyl chloride

A mixture of 6-[[6-(methylcarbamoyl)-1-naphthyl]oxy]pyridine-3-carboxylic acid (200 mg, 620.51 μmol, 1 eq), oxalyl chloride (118.14 mg, 930.77 μmol, 81.48 μL, 1.5 eq), DMF (4.54 mg, 62.05 μmol, 4.77 μL, 0.1 eq) in DCM (10 mL) was degassed at 0° C., and then the mixture was stirred at 25° C. for 2 hr under an N₂ atmosphere. The reaction mixture was concentrated under reduced pressure to give 6-[[6-(methylcarbamoyl)-1-naphthyl]oxy]pyridine-3-carbonyl chloride (215 mg, crude) as a yellow gum, which was used directly without further purification.

LCMS (ESI) [M+OMe]⁺ m/z: calc'd 337.11, found 337.1

### Step 6: (2S)-5,5-dimethyl-2-[[6-[[6-(methylcarbamoyl)-1-naphthyl]oxy]pyridine-3-carbonyl]amino]hexanoic acid

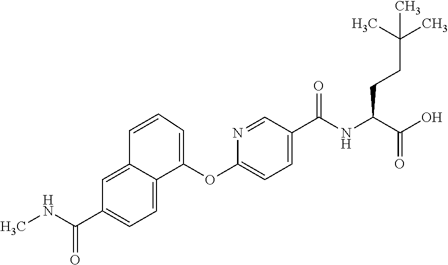 [SMILES: CNC(=O)c1ccc2c(Oc3ccc(C(=O)N[C@@H](CCC(C)(C)C)C(=O)O)cn3)cccc2c1]

To a mixture of (2S)-2-amino-5,5-dimethyl-hexanoic acid (100 mg, 628.04 μmol, 1 eq) and Na₂CO₃ (232.98 mg, 2.20 mmol, 3.5 eq) in H₂O (16 mL), dioxane (6 mL) was added 6-[[6-(methylcarbamoyl)-1-naphthyl]oxy]pyridine-3-carbonyl chloride (214.01 mg, 628.04 μmol, 1 eq). The mixture was stirred at 20° C. for 2 hr. The reaction mixture was poured into water (40 mL), extracted with EtOAc (40 mL×3). The combined water layers were lyophilized in vacuum to give the crude residue. The residue was purified by prep-HPLC (HCl). After purification, concentration and lyophilization, (2S)-5,5-dimethyl-2-[[6-[[6-(methylcarbamoyl)-1-naphthyl]oxy]pyridine-3-carbonyl]amino]hexanoic acid (92.3 mg, 31.55% yield, 99.5% purity) was obtained as a white solid.

LCMS (ESI) [M+H]⁺ m/z: calc'd 464.21, found 464.2

¹H NMR (400 MHZ, DMSO-d₆) δ: ppm 8.61-8.72 (2H, m) 8.48-8.56 (2H, m) 8.34 (1H, dd, J=8.53, 2.51 Hz) 7.96 (1H, d, J=8.28 Hz) 7.80-7.93 (2H, m) 7.64 (1H, t, J=7.91 Hz) 7.43 (1H, d, J=7.03 Hz) 7.29 (1H, d, J=8.53 Hz) 4.16-4.41 (1H, m) 2.83 (2H, d, J=4.52 Hz) 2.74-2.98 (1H, m) 1.62-1.90 (2H, m) 1.09-1.45 (2H, m) 0.86 (9H, s)

### SB004

### 2-((6-methyl-4-(methylcarbamoyl)pyridin-2-yl)carbamoyl)-5-(trifluoromethyl)benzoic acid

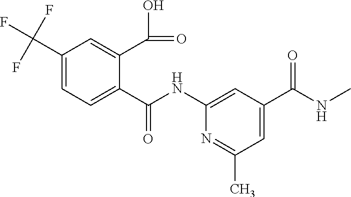 [SMILES: CNC(=O)c1cc(C)nc(NC(=O)c2ccc(C(F)(F)F)cc2C(=O)O)c1]

### Step 1:2-chloro-N, 6-Dimethylisonicotinamide

To 2-chloro-6-methyl-pyridine-4-carboxylic acid (5 g, 29.14 mmol, 1 eq) in DMF (200 mL) was added methanamine (9.84 g, 145.70 mmol, 5 eq, HCl), TEA (14.74 g, 145.70 mmol, 20.28 mL, 5 eq) and HATU (22.16 g, 58.28 mmol, 2 eq). The reaction was stirred at 20° C. for 2 h under N₂. The reaction was quenched by H₂O (800 mL). The aqueous layer was extracted with EtOAc (3×200 mL). The combined organic layers were washed with brine (2000 mL), dried over anhydrous Na₂SO₄, filtered and concentrated in vacuo. The residue was purified by silica gel chromatography eluted with (PE:EtOAc=20:1 to 1:1). 2-chloro-N, 6-dimethyl-pyridine-4-carboxamide (4.4 g, 66.90% yield, 81.8% purity) as a yellow solid was obtained.

LCMS (ESI) [M+H]⁺ m/z: calc'd 185.0, found 185.4

H NMR (400 MHZ, CHLOROFORM-d) □: ppm 7.47 (1H, s) 7.43 1H, s) 7.14 (1H, br s) 2.97 (3H, d, J=4.9 Hz) 2.54 (3H, s)

### Step 2: 2-((Diphenylmethylene)amino)-N,6-dimethylisonicotinamide

2-chloro-N,6-dimethyl-pyridine-4-carboxamide (3.4 g, 18.42 mmol, 1 eq) and diphenylmethanimine (4.34 g, 23.94 mmol, 4.02 mL, 1.3 eq) in toluene (85 mL) was added t-BuONa (4.42 g, 46.04 mmol, 2.5 eq) and [1-(2-diphenylphosphanyl-1-naphthyl)-2-naphthyl]-diphenyl-phosphane (344.01 mg, 552.48 μmol, 0.03 eq), Pd₂(dba)₃ (168.64 mg, 184.16 μmol, 0.01 eq). The reaction was stirred at 90° C. under N₂ for 16 h. The reaction was concentrated in vacuum. The residue was purified by silica gel chromatography eluted with (PE:EtOAc=10:1 to 1:3). 2-(benzhydrylideneamino)-N,6-dimethyl-pyridine-4-carboxamide (600 mg, 90% yield, 90% purity) as a yellow solid was obtained.

LCMS (ESI) [M+H]⁺ m/z: calc'd 330.1, found 330.1

### Step 3:2-Amino-N,6-dimethylisonicotinamide

2-(benzhydrylideneamino)-N,6-dimethyl-pyridine-4-carboxamide (600 mg, 1.82 mmol, 1 eq) in THF (5 mL) and HCl/H₂O (5 mL, 10% purity) was stirred at 20° C. for 16 hrs. The reaction was washed with EtOAc (10 mL). The residue was adjusted to pH 10 with saturated aqueous NaHCO₃ and the aqueous layer was concentrated in vacuo. DCM (10 mL) was added to the residue and stirred for 10 min. The suspension was filtered and the filter cake was washed with DCM (10 mL×3). The combined filtrates were concentrated to dryness to give 2-amino-N,6-dimethyl-pyridine-4-carboxamide (190 mg, 59.99% yield, 95% purity) as a yellow solid.

LCMS (ESI) [M+H]⁺ m/z: calc'd 166.2, found 166.1

### Step 4:2-(1,3-Dioxo-5-(trifluoromethyl) isoindolin-2-yl)-N,6-dimethylisonicotinamide

2-amino-N,6-dimethyl-pyridine-4-carboxamide (170 mg, 1.03 mmol, 1 eq) and 5-(trifluoromethyl) isobenzofuran-1,3-dione (222.40 mg, 1.03 mmol, 1 eq) in AcOH (4 mL) was stirred at 120° C. for 16 h under N₂. The reaction was concentrated and the residue was purified by silica gel chromatography eluted with (PE:EtOAc=5:1 to 1:3). 2-[1,3-dioxo-5-(trifluoromethyl) isoindolin-2-yl]-N,6-dimethyl-pyridine-4-carboxamide (188 mg, 41.23% yield, 82% purity) as a yellow solid was obtained.

LCMS (ESI) [M+H]⁺ m/z: calc'd 364.1, found 364.0

### Step 5: 2-((6-Methyl-4-(methylcarbamoyl)pyridin-2-yl)carbamoyl)-5-(trifluoromethyl)benzoic acid

To 2-[1,3-dioxo-5-(trifluoromethyl) isoindolin-2-yl]-N, 6-dimethyl-pyridine-4-carboxamide (210 mg, 578.05 μmol, 1 eq) in THF (4 mL) and H₂O (4 mL) was added LiOH·H₂O (72.77 mg, 1.73 mmol, 3 eq), and stirred at 20° C. for 1 hr under N₂. The reaction was concentrated and the residue was purified by prep. HPLC (column: Phenomenex Gemini-NX 150*30 mm*5 um; mobile phase: [water (0.05% ammonia hydroxide v/v)-ACN]; B %: 4%-44%, 14 min). After prep. HPLC purification, the eluent was lyophilized to give 2-[[6-methyl-4-(methylcarbamoyl)-2-pyridyl]carbamoyl]-5-(trifluoromethyl)benzoic acid (14 mg, 5.88% yield, 96.7% purity) as a white solid.

LCMS (ESI) [M+H]⁺ m/z: calc'd 382.1, found 382.0

¹H NMR (400 MHZ, DMSO-d₆) □: ppm 13.09 (1H, br s) 8.66 (1H, br d, J=4.3 Hz) 8.39 (1H, s) 7.88-8.01 (2H, m) 7.74 (1H, br d, J=8.0 Hz) 7.30 (3H, s) 2.79 (3H, d, J=4.4 Hz) 2.45 (3H, s)

### SB007

### (2S)-5,5-dimethyl-2-[[6-[3-(methylcarbamoyl)phenoxy]pyridine-3-carbonyl]amino]hexanoic acid

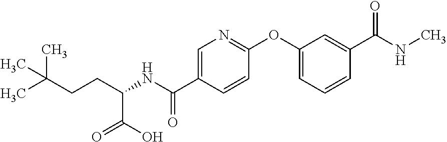 [SMILES: CNC(=O)c1cccc(Oc2ccc(C(=O)N[C@@H](CCC(C)(C)C)C(=O)O)cn2)c1]

### Step 1: Methyl 6-[3-(methylcarbamoyl)phenoxy]pyridine-3-carboxylate

A mixture of 3-hydroxy-N-methyl-benzamide (400 mg, 2.65 mmol, 1 eq), methyl 6-bromopyridine-3-carboxylate (571.66 mg, 2.65 mmol, 1 eq), potassium phosphate (561.70 mg, 2.65 mmol, 1 eq) and CuI (503.96 mg, 2.65 mmol, 1 eq) in DMSO (10 mL) was degassed and purged with N₂ three times, and then the mixture was stirred at 120° C. for 16 hr under an N₂ atmosphere. The mixture was washed with H₂O (40 mL) and extracted with EA (20 mL×4). The organic layer was dried with Na₂SO₄, filtered and concentrated under reduce pressure. The residue was purified by flash silica gel chromatography (ISCO®; 12 g SepaFlash® Silica Flash Column, eluent of 0˜100% ethyl acetate/petroleum ether gradient at 25 mL/min). Methyl 6-[3-(methylcarbamoyl)phenoxy]pyridine-3-carboxylate (73.92% yield, 100% purity) was obtained as a white solid.

### Step 2:6-[3-(Methylcarbamoyl)phenoxy]pyridine-3-carboxylic acid

To a solution of methyl 6-[3-(methylcarbamoyl)phenoxy]pyridine-3-carboxylate (460 mg, 1.61 mmol, 1 eq) in a mixture of THF (15 mL) and H₂O (5 mL) was added LiOH·H₂O (134.84 mg, 3.21 mmol, 2 eq). The mixture was stirred at 25° C. for 2 hr. The mixture was concentrated under reduced pressure. The residue was acidified with HCl (1M) to pH 2 and extracted with EA (20 mL×5). The combined organic layers were dried with Na₂SO₄, filtered and concentrated in vacuo. 6-[3-(methylcarbamoyl)phenoxy]pyridine-3-carboxylic acid (82.29% yield) was obtained as a white solid.

### Step 3:6-[3-(Methylcarbamoyl)phenoxy]pyridine-3-carbonyl chloride

A mixture of 6-[3-(methylcarbamoyl)phenoxy]pyridine-3-carboxylic acid (0.1 g, 367.30 μmol, 1 eq), oxalyl chloride (69.93 mg, 550.95 μmol, 48.23 μL, 1.5 eq), DMF (2.68 mg, 36.73 μmol, 2.83 μL, 0.1 eq) in DCM (5 mL) was degassed at 0° C., and then the mixture was stirred at 25° C. for 2 hr under an N₂ atmosphere. The reaction mixture was concentrated under reduced pressure. 6-[3-(methylcarbamoyl)phenoxy]pyridine-3-carbonyl chloride (91.29 mg, crude) was obtained as a yellow gum, which was used directly without further purification.

### Step 4: (2S)-5,5-Dimethyl-2-[[6-[3-(methylcarbamoyl)phenoxy]pyridine-3-carbonyl]amino]hexanoic acid

To a mixture of (2S)-2-amino-5,5-dimethyl-hexanoic acid (50 mg, 314.02 μmol, 1 eq) and Na₂CO₃ (116.49 mg, 1.10 mmol, 3.5 eq) in dioxane (8 mL), H₂O (3 mL) was added 6-[3-(methylcarbamoyl)phenoxy]pyridine-3-carbonyl chloride (91.29 mg, 314.02 μmol, 1 eq). The mixture was stirred at 20° C. for 2 hr. The reaction mixture was poured into water (40 mL) and extracted with EtOAc (40 mL×3). The combined water layers were lyophilized in vacuo to give the crude residue. After purification, concentration and lyophilization, (2S)-5,5-dimethyl-2-[[6-[3-(methylcarbamoyl)phenoxy]pyridine-3-carbonyl]amino]hexanoic acid (11 mg, 8.15% yield, 96.2% purity) was obtained as a white solid.

LCMS (ESI) [M+H]⁺ m/z: calc'd 413.20, found 414.1

¹H NMR (400 MHZ, DMSO-d₆) δ: ppm 0.87 (s, 9H) 1.17-1.35 (m, 2H) 1.65-1.85 (m, 2H) 2.77 (d, J=4.5 Hz, 3H) 4.31 (td, J=8.4, 5.3 Hz, 1H) 7.17 (d, J=8.5 Hz, 1H) 7.34 (dd, J=8.0, 1.8 Hz, 1H) 7.53 (t, J=7.9 Hz, 1H) 7.61 (t, J=1.9 Hz, 1H) 7.72 (d, J=7.8 Hz, 1H) 8.31 (dd, J=8.7, 2.4 Hz, 1H) 8.50 (br d, J=4.5 Hz, 1H) 8.63 (d, J=2.3 Hz, 1H) 8.71 (d, J=7.8 Hz, 1H)

### SB008

### (2S)-2-[[6-[3-[1-(2-methoxyethyl)triazol-4-yl]phenoxy]pyridine-3-carbonyl]amino]-5,5-dimethyl-hexanoic acid

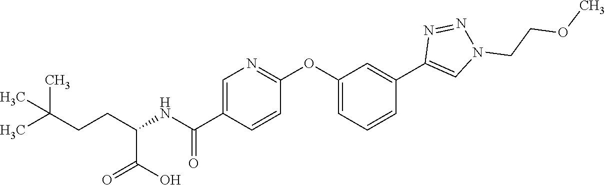 [SMILES: COCCn1cc(-c2cccc(Oc3ccc(C(=O)N[C@@H](CCC(C)(C)C)C(=O)O)cn3)c2)nn1]

### Step 1:3-[1-(2-Methoxyethyl)triazol-4-yl]phenol

To a solution of 3-ethynylphenol (576.00 mg, 4.88 mmol, 1.1 eq), 1-azido-2-methoxy-ethane (450 mg, 4.45 mmol, 1 eq), TEA (450.37 mg, 4.45 mmol, 619.48 μL, 1 eq) in tert-butanol (10 mL) and H₂O (10 mL) was added CuSO₄·5H₂O (277.82 mg, 1.11 mmol, 0.25 eq), sodium (2R)-2-[(1S)-1,2-dihydroxyethyl]-4-hydroxy-5-oxo-2H-furan-3-olate (440.86 mg, 2.23 mmol, 0.5 eq), and the mixture was stirred at 20° C. for 2 hrs. The reaction was filtered through celite and poured water (40 mL), extracted with EtOAc (40 mL×3). The combined organic layers were washed with brine (40 mL), dried over anhydrous Na₂SO₄, filtered and concentrated in vacuo to give the crude residue. The residue was purified by silica gel chromatography eluted with (PE:EtOAc=20:1 to 1:3). 3-[1-(2-methoxyethyl)triazol-4-yl]phenol (1.0 g, 97.36% yield, 95% purity) was obtained as a white solid.

¹H NMR (400 MHZ, CDCl₃) δ: ppm 7.87 (1H, s) 7.62 (1H, br s) 7.12-7.33 (2H, m) 6.82-6.98 (1H, m) 5.31 (1H, s) 4.57 (2H, t, J=4.89 Hz) 3.79 (2H, t, J=5.02 Hz) 3.37 (3H, s)

### Step 2:6-[3-[1-(2-Methoxyethyl)triazol-4-yl]phenoxy]pyridine-3-carboxylate

A mixture of 3-[1-(2-methoxyethyl)triazol-4-yl]phenol (800 mg, 3.65 mmol, 1 eq), methyl 6-bromopyridine-3-carboxylate (788.00 mg, 3.65 mmol, 1 eq) and CuI (69.49 mg, 364.90 μmol, 0.1 eq), K₃PO₄ (1.55 g, 7.30 mmol, 2 eq) in DMSO (10 mL) was degassed and purged with N₂ three times, and then the mixture was stirred at 120° C. for 16 hr under an N₂ atmosphere. The reaction was poured into water (40 mL) and extracted with EtOAc (40 mL×3). The combined organic layers were washed with brine (40 mL), dried over anhydrous Na₂SO₄, filtered and concentrated in vacuo. The residue was purified by silica gel chromatography eluted with (PE:EtOAc=20:1 to 1:3). Methyl 6-[3-[1-(2-methoxyethyl)triazol-4-yl]phenoxy]pyridine-3-carboxylate (660 mg, 51.04% yield) was obtained as a white solid.

¹H NMR (400 MHZ, CDCl₃) δ: ppm 8.83 (1H, d, J=2.01 Hz) 8.29 (1H, dd, J=8.66, 2.38 Hz) 7.91 (1H, s) 7.62-7.81 (2H, m) 7.49 (1H, t, J=7.91 Hz) 7.09-7.20 (1H, m) 6.98 (1H, d, J=8.78 Hz) 4.50-4.66 (2H, m) 3.92 (3H, s) 3.73-3.85 (2H, m) 3.37 (3H, s)

### Step 3:6-[3-[1-(2-Methoxyethyl)triazol-4-yl]phenoxy]pyridine-3-carboxylic acid

To a solution of methyl 6-[3-[1-(2-methoxyethyl)triazol-4-yl]phenoxy]pyridine-3-carboxylate (170 mg, 479.74 μmol, 1 eq) in a mixture of THF (3 mL) and H₂O (1 mL) was added LiOH·H₂O (40.26 mg, 959.48 μmol, 2 eq), The mixture was stirred at 25° C. for 2 hr. The mixture was concentrated under reduce pressure and the residue was acidified with HCl (1M) to pH 2. The residue was extracted with EA (20 mL×5). The combined organic layers were dried with Na₂SO₄, filtered and concentrated in vacuo. 6-[3-[1-(2-methoxyethyl)triazol-4-yl]phenoxy]pyridine-3-carboxylic acid (140 mg, 84.03% yield, 98% purity) was obtained as a yellow solid.

¹H NMR (400 MHZ, METHANOL-d₄) δ: ppm 8.74 (1H, d, J=2.01 Hz) 8.31-8.39 (2H, m) 7.71-7.78 (1H, m) 7.61-7.67 (1H, m) 7.52 (1H, t, J=7.91 Hz) 7.15 (1H, ddd, J=8.16, 2.38, 0.75 Hz) 7.07 (1H, d, J=8.53 Hz) 4.61 (2H, t, J=5.02 Hz) 3.76-3.99 (2H, m) 3.35 (3H, s)

### Step 4:6-[3-[1-(2-Methoxyethyl)triazol-4-yl]phenoxy]pyridine-3-carbonyl chloride

A mixture of 6-[3-[1-(2-methoxyethyl)triazol-4-yl]phenoxy]pyridine-3-carboxylic acid (140 mg, 411.36 μmol, 1 eq), oxalyl chloride (78.32 mg, 617.04 μmol, 54.01 μL, 1.5 eq), DMF (3.01 mg, 41.14 μmol, 3.17 μL, 0.1 eq) in DCM (5 mL) was degassed at 0° C., and then the mixture was stirred at 25° C. for 2 hr under an N₂ atmosphere. The reaction mixture was concentrated under reduced pressure to give 6-[3-[1-(2-methoxyethyl)triazol-4-yl]phenoxy]pyridine-3-carbonyl chloride (150 mg, crude) which was used directly without further purification.

### Step 5: (2S)-2-[[6-[3-[1-(2-Methoxyethyl)triazol-4-yl]phenoxy]pyridine-3-carbonyl]amino]-5,5-dimethyl-hexanoic acid

To a mixture of (2S)-2-amino-5,5-dimethyl-hexanoic acid (50 mg, 314.02 μmol, 0.75 eq) and Na₂CO₃ (155.09 mg, 1.46 mmol, 3.5 eq) in H₂O (8 mL) and dioxane (3 mL) was added 6-[3-[1-(2-methoxyethyl)triazol-4-yl]phenoxy]pyridine-3-carbonyl chloride (150 mg, 418.09 μmol, 1 eq). The mixture was stirred at 20° C. for 2 hr. The reaction mixture was poured into water (40 mL) and extracted with EtOAc (40 mL×3). The combined water layers were lyophilized in vacuo. The residue was purified by prep-HPLC (HCl) to give (2S)-2-[[6-[3-[1-(2-methoxyethyl)triazol-4-yl]phenoxy]pyridine-3-carbonyl]amino]-5,5-dimethyl-hexanoic acid (42 mg, 20.65% yield, 99% purity) as a white solid.

LCMS (ESI) [M+H]⁺ m/z: calc'd 482.2, found 482.2

¹H NMR (400 MHZ, DMSO-d₆) δ: ppm 8.70 (1H, d, J=7.83 Hz) 8.65 (1H, d, J=1.96 Hz) 8.61 (1H, s) 8.32 (1H, dd, J=8.62, 2.51 Hz) 7.73-7.80 (1H, m) 7.63-7.67 (1H, m) 7.52 (1H, t, J=7.89 Hz) 7.11-7.20 (2H, m) 4.57 (2H, t, J=5.20 Hz) 4.23-4.43 (1H, m) 3.77 (2H, t, J=5.20 Hz) 3.26 (3H, s) 1.64-1.92 (2H, m) 1.16-1.38 (2H, m) 0.87 (9H, s)

### SB009

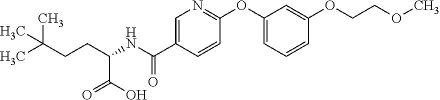 [SMILES: COCCOc1cccc(Oc2ccc(C(=O)N[C@@H](CCC(C)(C)C)C(=O)O)cn2)c1]

### Step 1: (3-acetoxyphenyl) acetate

To benzene-1,3-diol (1 g, 9.08 mmol, 1.52 mL, 1 eq) in Ac₂O (14.83 g, 145.31 mmol, 13.61 mL, 16 eq) was added p-toluenesulfonyl chloride (173.14 mg, 908.18 μmol, 0.1 eq) at 20° C. under N₂. The reaction was stirred at 20° C. for 1.5 h. The reaction was quenched by 10% NaOH/H₂O (45 mL) and extracted with DCM (3×30 mL). The combined organic layers were washed with brine (50 mL), dried over anhydrous Na₂SO₄, filtered and concentrated in vacuo. The residue was purified by silica gel chromatography eluted with (PE:EtOAc=50:1 to 10:1). (3-acetoxyphenyl) acetate (1.6 g, 90.73% yield) as a colourless oil was obtained.

LCMS (ESI) [M+H]⁺ m/z: calc'd 195.0, found 194.8

¹H NMR (400 MHZ, CHLOROFORM-d) δ: ppm 7.38 (1H, t, J=8.1 Hz) 6.99 (2H, dd, J=8.2, 2.2 Hz) 6.85-6.95 (1H, m) 2.22-2.36 (6H, m)

### Step 2: (3-hydroxyphenyl) acetate

To a mixture of (3-acetoxyphenyl) acetate (1.6 g, 8.24 mmol, 1 eq) and benzene-1,3-diol (907.27 mg, 8.24 mmol, 1.37 mL, 1 eq) in DMSO (10 mL) was added K₂CO₃ (1.14 g, 8.24 mmol, 1 eq) in one portion at 20° C. under N₂. The mixture was stirred at 20° C. for 4 h. TLC showed the reaction was completed. The mixture was poured into water (20 mL) and the aqueous phase was extracted with ethyl acetate (10 mL×6). The combined organic phase was washed with brine (20 mL), dried with anhydrous Na₂SO₄, filtered and concentrated in vacuo. The residue was purified by silica gel chromatography (petroleum ether/ethyl acetate=10/1 to 2/1). (3-hydroxyphenyl) acetate (1.9 g, 75.78% yield) as colorless oil was obtained.

¹H NMR (400 MHZ, CHLOROFORM-d) δ: ppm 7.21 (1H, t, J=8.1 Hz) 6.61-6.72 (2H, m) 6.56 (1H, t, J=2.3 Hz) 2.27-2.36 (3H, m)

### Step 3:3-(2-methoxyethoxy) phenol

To a mixture of (3-hydroxyphenyl) acetate (1.7 g, 11.17 mmol, 1 eq) and 2-methoxyethyl 4-methylbenzenesulfonate (2.32 g, 10.06 mmol, 0.9 eq) in DMF (30 mL) was added K₂CO₃ (3.40 g, 24.58 mmol, 2.2 eq) in one portion at 20° C. under N₂. The mixture was stirred at 80° C. for 4 hours. To the reaction mixture was added NaOH (20%, 32 mL) and the mixture was stirred at 0° C. for 15 minutes. The aqueous layer was basified with HCl/H₂O (1 M, 240 mL) until pH 5, and extracted with DCM (3×30 mL). The combined organic layers were washed with brine (100 mL), dried over anhydrous Na₂SO₄, filtered and concentrated in vacuo. The residue was combined with that of ES17843-53-P1A and purified by silica gel chromatography eluted with (PE:EA=100:1 to 3:1). 3-(2-methoxyethoxy) phenol (1.04 g, 55.34% yield) as a yellow oil was obtained.

¹H NMR (400 MHZ, CHLOROFORM-d) δ: ppm 7.12 (1H, t, J=8.0 Hz) 6.22-6.55 (3H, m) 5.24 (1H, br s) 4.10 (2H, dd, J=5.4, 3.9 Hz) 3.70-3.82 (2H, m) 3.41-3.51 (3H, m)

### Step 4: Methyl 6-[3-(2-methoxyethoxy)phenoxy]pyridine-3-carboxylate and 6-[3-(2-methoxyethoxy)phenoxy]pyridine-3-carboxylic acid

To a mixture of 3-(2-methoxyethoxy) phenol (916 mg, 5.45 mmol, 1 eq) and methyl 6-bromopyridine-3-carboxylate (1.18 g, 5.45 mmol, 1 eq) in DMSO (12 mL) was added CuI (103.72 mg, 544.62 μmol, 0.1 eq) and K₃PO₄ (2.31 g, 10.89 mmol, 2 eq) under N₂. The reaction was stirred at 120° C. for 16 hrs. To the reaction was added 100 mL of H₂O, then the mixture was filtered and the filter cake was washed with 30 mL of EtOAc. The filtrate was extracted with EtOAc (3×30 mL). The combined organic layers were washed with brine (20 mL), dried over anhydrous Na₂SO₄, filtered and concentrated in vacuo. The crude product was purified by silica gel chromatography eluted with (PE:EtOAc=20:1 to 1:1) to give methyl 6-[3-(2-methoxyethoxy)phenoxy]pyridine-3-carboxylate (260 mg, 857.21 μmol, 15.74% yield) as a yellow oil. The residual aqueous layer was adjusted with 1 M HCl/H₂O until pH 2, then extracted with EtOAc (3×30 mL). The combined organic layers were washed with brine (30 mL), dried over anhydrous Na₂SO₄, filtered and concentrated in vacuo to give crude 6-[3-(2-methoxyethoxy)phenoxy]pyridine-3-carboxylic acid (500 mg, 31.74% yield).

¹H NMR (400 MHZ, CHLOROFORM-d) δ: ppm 8.84 (1H, dd, J=2.3, 0.6 Hz) 8.27 (1H, dd, J=8.6, 2.4 Hz) 7.30-7.38 (1H, m) 7.05-7.19 (1H, m) 6.81-6.97 (1H, m) 6.70-6.78 (1H, m) 6.37-6.56 (1H, m) 4.05-4.19 (2H, m) 3.93 (2H, s) 3.75 (2H, td, J=4.7, 2.2 Hz) 3.46 (3H, d, J=1.7 Hz)

### Step 5:6-[3-(2-methoxyethoxy)phenoxy]pyridine-3-carboxylic acid

To a mixture of methyl 6-[3-(2-methoxyethoxy)phenoxy]pyridine-3-carboxylate (260 mg, 857.21 μmol, 1 eq) in THF (4.5 mL) and H₂O (1.5 mL) was added LiOH·H₂O (71.94 mg, 1.71 mmol, 2 eq). The reaction was stirred at 20° C. for 2 hrs. The mixture was concentrated under reduced pressure and the residue was acidified with HCl (1M) to pH 2. The residue was extracted with EtOAc (20 mL×5). The combined organic layers were dried with Na₂SO₄, filtered and concentrated in vacuo. The residue was purified by silica gel chromatography eluted with (DCM:MeOH=50:1 to 10:1) to give 6-[3-(2-methoxyethoxy)phenoxy]pyridine-3-carboxylic acid (247.9 mg, 99.97% yield) as a white oil.

LCMS (ESI) [M+H]⁺ m/z: calc'd 290.1, found 289.9

¹H NMR (400 MHZ, CHLOROFORM-d) δ: ppm 8.94 (1H, d, J=2.3 Hz) 8.34 (1H, dd, J=8.7, 2.4 Hz) 7.30-7.41 (1H, m) 6.97 (1H, d, J=8.7 Hz) 6.85-6.91 (1H, m) 6.71-6.83 (2H, m) 4.10-4.17 (2H, m) 3.78 (2H, dd, J=5.4, 4.0 Hz) 3.43-3.51 (3H, m)

### Step 6:6-[3-(2-methoxyethoxy)phenoxy]pyridine-3-carbonyl chloride

To 6-[3-(2-methoxyethoxy)phenoxy]pyridine-3-carboxylic acid (100 mg, 345.68 μmol, 1 eq) in DCM (4 mL) was added DMF (2.53 mg, 34.57 μmol, 2.66 μL, 0.1 eq) and oxalyl chloride (65.82 mg, 518.52 μmol, 45.39 μL, 1.5 eq) at 0° C. under N₂. The reaction was stirred at 20° C. for 2 h. The residue was concentrated in vacuo to give 6-[3-(2-methoxyethoxy)phenoxy]pyridine-3-carbonyl chloride (106 mg, 99.65% yield) as a colorless oil.

LCMS (ESI) [M−Cl+OMe]⁺ m/z: calc'd 304.0, found 304.0

### Step 7: (2S)-2-[[6-[3-(2-methoxyethoxy)phenoxy]pyridine-3-carbonyl]amino]-5,5-dimethyl-hexanoic acid

To a solution of (2S)-2-amino-5, 5-dimethyl-hexanoic acid (50.00 mg, 314.02 μmol, 0.912 eq) and Na₂CO₃ (127.78 mg, 1.21 mmol, 3.5 eq) in dioxane (3 mL) and H₂O (8 mL) was added 6-[3-(2-methoxyethoxy)phenoxy]pyridine-3-carbonyl chloride (106 mg, 344.46 μmol, 1 eq). The reaction was stirred at 20° C. for 2 h. The reaction mixture was poured into water (10 mL) and extracted with EtOAc (10 mL×3). The combined water layers were lyophilized in vacuo to give the crude residue. The crude product was combined with that of (ES17843-67-P1A), and purified by prep. HPLC (column: Boston Green ODS 150*30 mm*5 um; mobile phase: [water (0.05% HCl)-ACN]; B %: 29%-69%, 9 min). After prep. HPLC purification, the residue was lyophilized to give (2S)-2-[[6-[3-(2-methoxyethoxyphenoxy]pyridine-3-carbonyl]amino]-5, 5-dimethyl-hexanoic acid (12 mg, 8.00% yield, 98.8% purity) as a white solid.

LCMS (ESI) [M+H]⁺ m/z: calc'd 431.2, found 431.2

¹H NMR (400 MHZ, DMSO-d₆) δ: ppm 8.68 (1H, d, J=7.8 Hz) 8.64 (1H, d, J=2.3 Hz) 8.29 (1H, dd, J=8.6, 2.4 Hz) 7.32 (1H, t, J=8.2 Hz) 7.08 (1H, d, J=8.7 Hz) 6.83 (1H, dd, J=8.2, 2.1 Hz) 6.76 (1H, t, J=2.3 Hz) 6.72 (1H, dd, J=7.8, 1.8 Hz) 4.23-4.38 (1H, m) 4.23-4.38 (1H, m) 4.06-4.11 (2H, m) 3.64-3.66 (2H, m) 3.29 (3H, s) 1.62-1.85 (2H, m) 1.13-1.38 (2H, m) 0.87 (9H, s)

### SB010

### (2S)-2-[[6-[3-[1-(2-acetamidoethyl)triazol-4-yl]phenoxy]pyridine-3-carbonyl]amino]-5,5-dimethyl-hexanoic acid

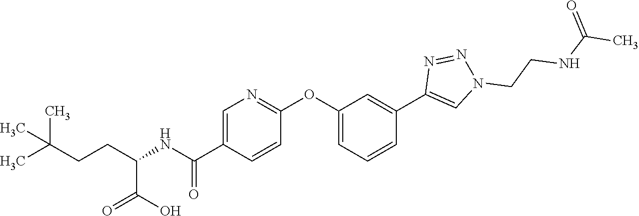 [SMILES: CC(=O)NCCn1cc(-c2cccc(Oc3ccc(C(=O)N[C@@H](CCC(C)(C)C)C(=O)O)cn3)c2)nn1]

### Step 1: Tert-butyl N-[2-[4-(3-hydroxyphenyl)triazol-1-yl]ethyl]carbamate

To a solution of 3-ethynylphenol (600 mg, 5.08 mmol, 1.1 eq), tert-butyl N-(2-azidoethyl) carbamate (859.80 mg, 4.62 mmol, 1 eq), TEA (467.22 mg, 4.62 mmol, 642.67 μL, 1 eq) in tert-butanol (10 mL) and H₂O (10 mL) was added CuSO₄·5H₂O (288.22 mg, 1.15 mmol, 0.25 eq), sodium (2R)-2-[(1S)-1,2-dihydroxyethyl]-4-hydroxy-5-oxo-2H-furan-3-olate (457.37 mg, 2.31 mmol, 0.5 eq), and the mixture was stirred at 20° C. for 12 hrs. The reaction was filtered through celite and poured into water (40 mL) and extracted with EtOAc (40 mL×3). The combined organic layers were washed with brine (40 mL), dried over anhydrous Na₂SO₄, filtered and concentrated in vacuo. The residue was purified by column chromatography (SiO₂, petroleum ether/ethyl acetate=20/1 to 3/1). Tert-butyl N-[2-[4-(3-hydroxyphenyl)triazol-1-yl]ethyl]carbamate (1.2 g, 85.39% yield) was obtained as a white solid.

LCMS (ESI) [M+H]⁺ m/z: calc'd 304.1, found 305.1

### Step 2:3-[1-(2-aminoethyl)triazol-4-yl]phenol

To a solution of tert-butyl N-[2-[4-(3-hydroxyphenyl)triazol-1-yl]ethyl]carbamate (1 g, 3.29 mmol, 1 eq) in DCM (10 mL) was added TFA (3.75 g, 32.86 mmol, 2.43 mL, 10 eq). The reaction mixture was stirred under nitrogen at 20° C. for 17 hr. The reaction mixture was concentrated under reduced pressure directly. 3-[1-(2-aminoethyl)triazol-4-yl]phenol was obtained as a white solid.

### Step 3: N-[2-[4-(3-hydroxyphenyl)triazol-1-yl]ethyl]acetamide

To a solution of 3-[1-(2-aminoethyl)triazol-4-yl]phenol (700 mg, 3.43 mmol, 1 eq) and TEA (1.73 g, 17.14 mmol, 2.39 mL, 5 eq) in DCM (10 mL) was added acetyl chloride (269.05 mg, 3.43 mmol, 244.59 μL, 1 eq) drop-wise at 0° C. under N₂. The reaction mixture was warmed to 20° C. and stirred for 2 hours. The reaction was poured into water (40 mL) and extracted with DCM (40 mL×3). The combined organic layers were washed with brine (40 mL), dried over anhydrous Na₂SO₄, filtered and concentrated in vacuo. The residue was purified by prep. HPLC (basic condition) and the residual aqueous solution was lyophilized to give N-[2-[4-(3-hydroxyphenyl)triazol-1-yl]ethyl]acetamide (150 mg, 17.77% yield) as a pale yellow solid.

1H NMR (400 MHZ, DMSO-d6) δ: ppm 8.26-8.67 (m, 1H) 8.05 (br t, J=5.5 Hz, 1H) 7.27 (d, J=1.0 Hz, 1H) 7.20-7.24 (m, 2H) 6.63-6.76 (m, 1H) 4.42 (t, J=5.9 Hz, 2H) 3.51 (q, J=5.8 Hz, 2H) 1.67-1.88 (m, 3H).

### Step 4: Methyl 6-[3-[1-(2-acetamidoethyl)triazol-4-yl]phenoxy]pyridine-3-carboxylate

To a mixture of methyl 6-bromopyridine-3-carboxylate (105.27 mg, 487.28 μmol, 1.2 eq), N-[2-[4-(3-hydroxyphenyl)triazol-1-yl]ethyl]acetamide (100 mg, 406.07 μmol, 1 eq), potassium phosphate (86.19 mg, 406.07 μmol, 1 eq) and CuI (77.34 mg, 406.07 μmol, 1 eq) in DMSO (4 mL) was degassed and purged with N₂ three times, and then the mixture was stirred at 120° C. for 16 hr under N₂ atmosphere. The reaction mixture was extracted with EA (20 mL×4). The organic layer was dried with Na₂SO₄, filtered and concentrated under reduced pressure. The residue was purified by flash silica gel chromatography (ISCO®; 12 g SepaFlash® Silica Flash Column, eluent of 0˜100% ethyl acetate/petroleum ether gradient at 25 mL/min) to give methyl 6-[3-[1-(2-acetamidoethyl)triazol-4-yl]phenoxy]pyridine-3-carboxylate (90.40% yield) as a white solid.

¹H NMR (400 MHZ, DMSO-d6) δ: ppm 8.71 (d, J=2.0 Hz, 1H) 8.61 (s, 1H) 8.34 (dd, J=8.5, 2.5 Hz, 1H) 8.04 (br t, J=5.8 Hz, 1H) 7.76 (d, J=7.8 Hz, 1H) 7.63-7.66 (m, 1H) 7.53 (t, J=7.9 Hz, 1H) 7.13-7.21 (m, 2H) 4.43 (t, J=6.0 Hz, 2H) 3.86 (s, 3H) 3.50 (q, J=6.0 Hz, 2H) 1.76-1.79 (m, 3H)

### Step 5: 6-[3-[1-(2-Acetamidoethyl)triazol-4-yl]phenoxy]pyridine-3-carboxylic acid

To a solution of methyl 6-[3-[1-(2-acetamidoethyl)triazol-4-yl]phenoxy]pyridine-3-carboxylate (100 mg, 262.20 μmol, 1 eq) in a mixture of THF (0.6 mL) and H₂O (0.2 mL) was added LiOH·H₂O (22.01 mg, 524.41 μmol, 2 eq) and the mixture was stirred at 25° C. for 2 hr. The mixture was concentrated under reduced pressure and acidified with HCl (1M) to pH 2. The residue was extracted with EA (20 mL×5). The combined organic layers were dried with Na₂SO₄, filtered and concentrated in vacuo to give 6-[3-[1-(2-acetamidoethyl)triazol-4-yl]phenoxy]pyridine-3-carboxylic acid (93.44% yield, 90% purity) as a white solid.

¹H NMR (400 MHZ, DMSO-d6) δ: ppm 8.68 (d, J=2.0 Hz, 1H) 8.61 (s, 1H) 8.31 (dd, J=8.7, 2.4 Hz, 1H) 8.05 (br s, 1H) 7.75 (d, J=8.0 Hz, 1H) 7.63 (s, 1H) 7.53 (t, J=7.9 Hz, 1H) 7.16 (br d, J=8.8 Hz, 2H) 4.43 (br t, J=5.8 Hz, 2H) 3.51 (q, J=5.8 Hz, 2H) 1.78 (s, 3H)

### Step 6: (2,5-Dioxopyrrolidin-1-yl) 6-[3-[1-(2-acetamidoethyl)triazol-4-yl]phenoxy]pyridine-3-carboxylate

To a solution of 6-[3-[1-(2-acetamidoethyl)triazol-4-yl]phenoxy]pyridine-3-carboxylic acid (80 mg, 217.77 μmol, 1 eq) in DMF (2 mL) was added 1-hydroxypyrrolidine-2,5-dione (62.66 mg, 544.43 μmol, 2.5 eq) and EDCI (62.62 mg, 326.66 μmol, 1.5 eq), and the mixture was degassed three times and stirred under N₂ at 20° C. for 3 h. Water (4 mL) and ethyl acetate (5 mL) were added. The organic layer was washed with water (3 ml), brine (5 mL×2), dried over anhydrous sodium sulfate, filtered and concentrated. (2,5-dioxopyrrolidin-1-yl) 6-[3-[1-(2-acetamidoethyl)triazol-4-yl]phenoxy]pyridine-3-carboxylate (100 mg, crude) was obtained as a yellow oil.

### Step 7: (2S)-2-[[6-[3-[1-(2-acetamidoethyl)triazol-4-yl]phenoxy]pyridine-3-carbonyl]amino]-5,5-dimethyl-hexanoic acid

A mixture of (2,5-dioxopyrrolidin-1-yl)-6-[3-[1-(2-acetamidoethyl)triazol-4-yl]phenoxy]pyridine-3-carboxylate (80 mg, 172.25 μmol, 1 eq), (2S)-2-amino-5,5-dimethyl-hexanoic acid (27.43 mg, 172.25 μmol, 1 eq), and DIEA (33.39 mg, 258.38 μmol, 45.01 μL, 1.5 eq) in DCM (0.4 mL), DMF (2.4 mL), H₂O (1.2 mL) was degassed and purged with N₂ three times, and then the mixture was stirred at 20° C. for 4 hrs under N₂ atmosphere. The reaction mixture was concentrated in vacuo to give the crude residue which was purified with prep. HPLC (HCl condition). The residual aqueous solution was lyophilized to give (2S)-2-[[6-[3-[1-(2-acetamidoethyl)triazol-4-yl]phenoxy]pyridine-3-carbonyl]amino]-5,5-dimethyl-hexanoic acid (22 mg, 25.01% yield, 99.6% purity) as a white solid.

LCMS (ESI) [M+H]⁺ m/z: calc'd 508.24, found 509.3

¹H NMR (400 MHZ, DMSOd6) δ: 8.71 (d, J=7.8 Hz, 1H) 8.65 (d, J=2.5 Hz, 1H) 8.62 (s, 1H) 8.32 (dd, J=8.5, 2.5 Hz, 1H) 8.07 (br t, J=5.6 Hz, 1H) 7.74 (d, J=8.0 Hz, 1H) 7.60-7.65 (m, 1H) 7.52 (t, J=7.9 Hz, 1H) 7.11-7.19 (m, 2H) 4.43 (t, J=5.9 Hz, 2H) 4.31 (td, J=8.4, 5.0 Hz, 3H) 3.50 (q, J=6.0 Hz, 2H) 1.78 (s, 3H) 1.16-1.36 (m, 2H) 0.86 (s, 9H)

### SB011

### (2S)-2-[[6-[3-(2-acetamidoethoxy)phenoxy]pyridine-3-carbonyl]amino]-5,5-dimethyl-hexanoic acid

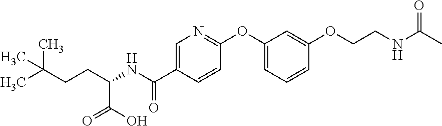 [SMILES: CC(=O)NCCOc1cccc(Oc2ccc(C(=O)N[C@@H](CCC(C)(C)C)C(=O)O)cn2)c1]

### Step 1: [3-[2-(tert-butoxycarbonylamino)ethoxy]phenyl]acetate

A mixture of (3-hydroxyphenyl) acetate (1.7 g, 11.17 mmol, 1.52 mL, 1 eq), tert-butyl N-(2-hydroxyethyl) carbamate (1.80 g, 11.18 mmol, 1.73 mL, 1 eq), PPh₃ (3.52 g, 13.41 mmol, 1.2 eq) in THF (30 mL) was degassed and purged with N₂ three times and DIAD (2.71 g, 13.41 mmol, 2.61 mL, 1.2 eq) at 0° C. was added, then the mixture was stirred at 20° C. for 16 hrs under an N₂ atmosphere. The reaction mixture was poured into water (40 mL) and extracted with EtOAc (40 mL×3). The combined organic layers were washed with brine (40 mL), dried over anhydrous Na₂SO₄, filtered and concentrated in vacuo. The residue was purified by silica gel chromatography eluted with (PE:EtOAc=20:1 to 3:1) to give [3-[2-(tert-butoxycarbonylamino)ethoxy]phenyl]acetate (1.9 g, 54.70% yield, 95% purity) as a colourless oil.

¹H NMR (400 MHZ, CDCl₃) δ: ppm 7.29 (1H, t, J=8.28 Hz) 6.78 (1H, dd, J=8.16, 2.13 Hz) 6.71 (1H, dd, J=8.16, 1.38 Hz) 6.65 (1H, t, J=2.26 Hz) 4.01 (2H, t, J=5.02 Hz) 3.54 (2H, br d, J=4.27 Hz) 2.31 (3H, s) 1.47 (9H, s)

### Step 2: Tert-butyl N-[2-(3-hydroxyphenoxy)ethyl]carbamate

To a mixture of [3-[2-(tert-butoxycarbonylamino)ethoxy]phenyl]acetate (1.7 g, 5.76 mmol, 1 eq) in MeOH (30 mL) was added K₂CO₃ (1.59 g, 11.51 mmol, 2 eq) and H₂O (6 mL), and the mixture was stirred at 20° C. for 2 hr. The reaction mixture was poured into 10% aq. HCl (80 mL) and extracted with EtOAc (40 mL×3). The combined organic layers were washed with brine (40 mL), dried over anhydrous Na₂SO₄, filtered and concentrated in vacuo. The residue was purified by column chromatography (SiO₂, petroleum ether/ethyl acetate=50/1 to 1/3) to give tert-butyl-N-[2-(3-hydroxyphenoxy)ethyl]carbamate (900 mg, 61.73% yield) as a colourless oil.

¹H NMR (400 MHZ, CDCl₃) δ: ppm 7.12 (1H, t, J=8.13 Hz) 6.40-6.54 (3H, m) 3.98 (2H, t, J=5.07 Hz) 3.52 (2H, br d, J=5.01 Hz) 1.47 (9H, s)

### Step 3: Methyl 6-[3-[2-(tert-butoxycarbonylamino)ethoxy]phenoxy]pyridine-3-carboxylate

A mixture of tert-butyl N-[2-(3-hydroxyphenoxy)ethyl]carbamate (100 mg, 394.80 μmol, 1 eq), methyl 6-fluoropyridine-3-carboxylate (74.00 mg, 477.03 μmol, 1.21 eq), Cs₂CO₃ (385.90 mg, 1.18 mmol, 3 eq) in DMF (3 mL) was degassed and purged with N₂ three times, and then the mixture was stirred at 20° C. for 16 hr under an N₂ atmosphere. The reaction mixture was poured into water (50 mL) and extracted with EA (3×30 mL). The combined organic layers were washed with brine (30 mL), dried over anhydrous Na₂SO₄, filtered and concentrated in vacuo. The residue was purified by silica gel chromatography eluted with (PE:EtOAc=100:1 to 1:2) to give methyl 6-[3-[2-(tert-butoxycarbonylamino)ethoxy]phenoxy]pyridine-3-carboxylate (120 mg, 70.43% yield, 90% purity) as a yellow solid.

LCMS (ESI) [M+H−100]⁺ m/z: calc'd 289.1, found 289.0

¹H NMR (400 MHZ, CDCl₃) δ: ppm 8.84 (1H, d, J=2.20 Hz) 8.29 (1H, dd, J=8.62, 2.38 Hz) 7.24-7.40 (1H, m) 6.95 (1H, d, J=8.68 Hz) 6.78 (2H, ddd, J=12.93, 8.22, 1.96 Hz) 6.69-6.73 (1H, m) 4.02 (2H, t, J=5.07 Hz) 3.93 (3H, s) 3.47-3.58 (2H, m) 1.46 (9H, s)

### Step 4: Methyl 6-[3-(2-aminoethoxy)phenoxy]pyridine-3-carboxylate

A mixture of methyl 6-[3-[2-(tert-butoxycarbonylamino)ethoxy]phenoxy]pyridine-3-carboxylate (120 mg, 308.95 μmol, 1 eq), HCl/dioxane (4 M, 772.37 μL, 10 eq) in dioxane (3 mL) was stirred at 20° C. for 2 hrs. The mixture was concentrated in vacuo to give methyl 6-[3-(2-aminoethoxy)phenoxy]pyridine-3-carboxylate (90 mg, 89.70% yield, HCl salt) as a white solid, which was used directly without purification.

¹H NMR (400 MHZ, DMSO-d₆) δ: ppm 8.70 (1H, d, J=2.26 Hz) 8.32 (4H, br dd, J=8.66, 2.38 Hz) 7.39 (1H, t, J=8.16 Hz) 7.14 (1H, d, J=8.78 Hz) 6.91 (1H, dd, J=8.16, 1.88 Hz) 6.77-6.86 (2H, m) 4.20 (2H, t, J=5.14 Hz) 3.86 (3H, s) 3.15-3.26 (2H, m)

### Step 5: Methyl 6-[3-(2-acetamidoethoxy)phenoxy]pyridine-3-carboxylate

To a mixture of methyl 6-[3-(2-aminoethoxy)phenoxy]pyridine-3-carboxylate (90 mg, 312.18 μmol, 1 eq) in THF (3 mL) was added Ac₂O (38.24 mg, 374.61 μmol, 35.09 μL, 1.2 eq) and Et₃N (94.77 mg, 936.53 μmol, 130.35 μL, 3 eq) and the mixture was stirred at 20° C. for 12 hrs. The reaction mixture was concentrated and poured into water (40 mL), then extracted with EtOAc (40 mL×3). The combined organic layers were washed with brine (40 mL), dried over anhydrous Na₂SO₄, filtered and concentrated in vacuo to give methyl 6-[3-(2-acetamidoethoxy)phenoxy]pyridine-3-carboxylate (80 mg, 77.58% yield) as a yellow oil, which was used directly without purification.

¹H NMR (400 MHZ, DMSO-d₆) δ: ppm 8.71 (1H, d, J=1.96 Hz) 8.31 (1H, dd, J=8.56, 2.45 Hz) 8.04-8.22 (1H, m) 7.35 (1H, t, J=8.13 Hz) 7.12 (1H, d, J=8.68 Hz) 6.86 (1H, dd, J=8.31, 1.83 Hz) 6.73-6.82 (2H, m) 3.98 (2H, t, J=5.69 Hz) 3.86 (3H, s) 3.39 (2H, q, J=5.62 Hz) 1.82 (3H, s)

### Step 6:6-[3-(2-acetamidoethoxy)phenoxy]pyridine-3-carboxylic acid

To a solution of methyl 6-[3-(2-acetamidoethoxy)phenoxy]pyridine-3-carboxylate (80 mg, 242.18 μmol, 1 eq) in a mixture of THF (3 mL) and H₂O (1 mL) was added LiOH·H₂O (20.32 mg, 484.36 μmol, 2 eq), The mixture was stirred at 25° C. for 12 hr. The mixture was concentrated under reduced pressure and the residue was acidified with HCl (1M) to pH 2. The residue was extracted with EA (20 mL×5) and the combined organic layers were dried with Na₂SO₄, filtered and concentrated in vacuo to give 6-[3-(2-acetamidoethoxy)phenoxy]pyridine-3-carboxylic acid (70 mg, 24% yield, 90% purity) as a yellow oil.

¹H NMR (400 MHZ, DMSO-d₆) δ: ppm 8.68 (1H, d, J=2.26 Hz) 8.29 (1H, dd, J=8.66, 2.38 Hz) 8.02-8.15 (1H, m) 7.35 (1H, t, J=8.16 Hz) 7.09 (1H, d, J=8.53 Hz) 6.85 (1H, dd, J=8.28, 2.01 Hz) 6.73-6.80 (2H, m) 3.96-4.01 (2H, m) 3.40-3.42 (2H, m) 1.82 (3H, s)

### Step 7: (2,5-dioxopyrrolidin-1-yl) 6-[3-(2-acetamidoethoxy)phenoxy]pyridine-3-carboxylate

To a solution of 1-hydroxypyrrolidine-2,5-dione (63.67 mg, 553.26 μmol, 2.5 eq) in DMF (5 mL), was added 6-[3-(2-acetamidoethoxy)phenoxy]pyridine-3-carboxylic acid (70 mg, 221.30 μmol, 1 eq) and EDCI (63.64 mg, 331.95 μmol, 1.5 eq) and the mixture was degassed three times and stirred under N₂ at 20° C. for 3 h. The reaction mixture was poured into water (40 mL) and extracted with EtOAc (40 mL×3). The combined organic layers were washed with brine (40 mL), dried over anhydrous Na₂SO₄, filtered and concentrated in vacuo to give (2,5-dioxopyrrolidin-1-yl)-6-[3-(2-acetamidoethoxy)phenoxy]pyridine-3-carboxylate (85 mg, 74.33% yield, 80% purity) as a yellow oil, which was used directly without purification.

LCMS (ESI) [M+H]⁺ m/z: calc'd 414.1, found 414.0

### Step 8: (2S)-2-[[6-[3-(2-acetamidoethoxy)phenoxy]pyridine-3-carbonyl]amino]-5,5-dimethyl-hexanoic acid

A mixture of (2,5-dioxopyrrolidin-1-yl)-6-[3-(2-acetamidoethoxy)phenoxy]pyridine-3-carboxylate (85 mg, 205.62 μmol, 1 eq), (2S)-2-amino-5,5-dimethyl-hexanoic acid (33.15 mg, 208.20 μmol, 1.01 eq) and DIEA (26.58 mg, 205.62 μmol, 35.82 uL, 1 eq) in DCM (0.5 mL), H2O (1.5 mL) and DMF (3 mL) were degassed and purged with N₂ three times, and then the mixture was stirred at 25° C. for 16 hrs under an N₂ atmosphere. The reaction was concentrated in vacuo and he crude product was purified by Prep. HPLC (HCl) column: Boston Green ODS 150*30 mm*5 um; mobile phase: [water (0.05% HCl)-ACN]; B %: 24%-64%,9 min. (2S)-2-[[6-[3-(2-acetamidoethoxy)phenoxy]pyridine-3-carbonyl]amino]-5,5-dimethyl-hexanoic acid (16.5 mg, 15.79% yield, 90% purity) was obtained as a white solid.

LCMS (ESI) [M+H]⁺ m/z: calc'd 458.22, found 458.2

¹H NMR (400 MHZ, DMSO-d₆) δ: ppm 8.68 (1H, d, J=7.83 Hz) 8.64 (1H, d, J=2.08 Hz) 8.29 (1H, dd, J=8.62, 2.51 Hz) 8.03-8.13 (1H, m) 7.33 (1H, t, J=8.13 Hz) 7.09 (1H, d, J=8.93 Hz) 6.80-6.92 (1H, m) 6.70-6.77 (2H, m) 4.31 (1H, td, J=8.47, 5.20 Hz) 3.97 (2H, t, J=5.69 Hz) 3.39 (2H, q, J=5.62 Hz) 1.81 (3H, s) 1.77 (1H, br t, J=5.14 Hz) 1.11-1.37 (3H, m) 0.87 (9H, s)

### SB012

### (2S)-2-[[6-[3-(acetamidomethyl)phenoxy]pyridine-3-carbonyl]amino]-5,5-dimethyl-hexanoic acid

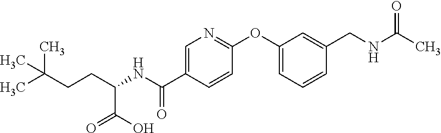 [SMILES: CC(=O)NCc1cccc(Oc2ccc(C(=O)N[C@@H](CCC(C)(C)C)C(=O)O)cn2)c1]

### Step 1: N-[(3-hydroxyphenyl)methyl] acetamide

To a solution of 3-(aminomethyl) phenol (800 mg, 6.50 mμuλ, 1 eq) and Et₃N (1.64 g, 16.2 mmol, 2.26 mL, 2.5 eq) in DCM (20 mL) was added acetyl chloride (509 mg, 6.50 mmol, 463 μL, 1.0 eq) drop-wise at 0° C. under N₂. The reaction mixture was warmed to 20° C. and stirred for 2 hours. The reaction mixture was filtered and the filter cake washed with 25 mL of ethyl acetate. The combined organic layers were concentrated in vacuo and the residue was purified by reversed-phase HPLC (water (0.05% ammonia hydroxide v/v)-ACN; Column: YMC Triart C18 250*50 mm*7 μm). The product was lyophilized to give N-[(3-hydroxyphenyl)methyl] acetamide (720 mg, 65% yield, 97% purity) as a colourless oil.

¹H NMR (400 MHZ, CDCl₃) δ: 7.10 (t, J=7.82 Hz, 1H), 6.76 (s, 1H), 6.66-6.74 (m, 2H), 5.96 (br s, 1H), 4.30 (d, J=6.00 Hz, 2H), 1.95 (s, 3H).

### Step 2: Methyl 6-[3-(acetamidomethyl)phenoxy]pyridine-3-carboxylate

A mixture of N-[(3-hydroxyphenyl)methyl]acetamide (620 mg, 3.75 mmol, 1 eq), methyl 6-bromopyridine-3-carboxylate (810 mg, 3.75 mmol, 1 eq), potassium phosphate (796.7 mg, 3.75 mmol, 1 eq) and CuI (714 mg, 3.75 mmol, 1.0 eq) in DMSO (15 mL) was degassed and purged with N₂ three times, and then the mixture was stirred at 120° C. for 16 hr under an N₂ atmosphere. The mixture was washed with H₂O (40 mL) and extracted with ethyl acetate (20 mL×4). The organic layer was dried with Na₂SO₄, filtered and concentrated under reduced pressure. The residue was purified by flash silica gel chromatography (ISCO®; 12 g SepaFlash® Silica Flash Column, eluent of 0˜100% ethyl acetate/petroleum ether gradient at 25 mL/min) to give methyl 6-[3-(acetamidomethyl)phenoxy]pyridine-3-carboxylate (54% yield, 86% purity) as a yellow oil.

¹H NMR (400 MHZ, CDCl₃) δ: 8.70-8.77 (m, 1H), 8.22 (dd, J=8.63, 2.38 Hz, 1H), 7.33 (t, J=7.75 Hz, 1H), 7.11 (d, J=7.63 Hz, 1H), 6.97-7.05 (m, 2H), 6.89 (d, J=8.75 Hz, 1H), 5.83 (br s, 1H), 4.40 (d, J=5.75 Hz, 2H), 3.85 (s, 3H), 2.89 (s, 2H), 2.81 (s, 2H) 1.97 (s, 3H).

### Step 3:6-[3-(acetamidomethyl)phenoxy]pyridine-3-carboxylic acid

To a solution of methyl 6-[3-(acetamidomethyl)phenoxy]pyridine-3-carboxylate (610 mg, 2.0 mmol, 1 eq) in a mixture of THF (15 mL) and H₂O (5 mL) was added LiOH·H₂O (170 mg, 4.0 mmol, 2.0 eq). The mixture was stirred at 25° C. for 2 hr. The mixture was concentrated under reduced pressure and the residue was acidified with HCl (1M) to pH 2. The residue was extracted with ethyl acetate (20 mL×5) and the combined organic layers were dried with Na₂SO₄, filtered and concentrated in vacuo to give 6-[3-(acetamidomethyl)phenoxy]pyridine-3-carboxylic acid (500 mg, 86% yield, 100% purity) as a yellow oil.

¹H NMR (400 MHZ, METHANOL-d4) δ: 8.84 (d, J=1.75 Hz, 1H), 8.31 (dd, J=8.63, 2.38 Hz, 1H), 7.40 (t, J=7.82 Hz, 1H), 7.18 (d, J=7.63 Hz, 1H), 7.04-7.11 (m, 2H), 6.98 (d, J=8.76 Hz, 1H), 5.95 (br s, 1H), 4.47 (d, J=5.75 Hz, 2H), 2.05 (s, 3H), 1.26 (t, J=7.13 Hz, 2H).

### Step 4: (2,5-dioxopyrrolidin-1-yl) 6-[3 (acetamidomethyl)phenoxy]pyridine-3-carboxylate

To a solution of 6-[3-(acetamidomethyl)phenoxy]pyridine-3-carboxylic acid (50 mg, 174 μmol, 1 eq) in DMF (2 mL) was added 1-hydroxypyrrolidine-2,5-dione (50 mg, 436 μmol, 2.5 eq) and EDCI (50 mg, 261 μmol, 1.5 eq), and the mixture was degassed three times and stirred under N₂ at 20° C. for 3 h. The reaction mixture was poured into water (4 mL) and extracted with ethyl acetate (5 mL). The organic layer washed with water (3 ml), brine (5 mL×2), dried over anhydrous sodium sulfate, filtered and concentrated in vacuo to give (2,5-dioxopyrrolidin-1-yl)-6-[3 (acetamidomethyl) phenoxy]pyridine-3-carboxylate (50 mg, 68% yield, 92% purity) as a yellow oil.

LCMS (ESI) [M+H]⁺ m/z: calc'd 383.1, found 384.0

### Step 5: (2S)-2-[[6-[3-(acetamidomethyl)phenoxy]pyridine-3 carbonyl]amino]-5,5-dimethyl-hexanoic acid

A mixture of (2,5-dioxopyrrolidin-1-yl) 6-[3-(acetamidomethyl)phenoxy]pyridine-3 carboxylate (50 mg, 130 μmol, 1 eq), (2S)-2-amino-5,5-dimethyl-hexanoic acid (21 mg, 130 μmol, 1 eq), and DIEA (25 mg, 195 μmol, 34 μL, 1.5 eq) in DCM (0.2 mL), DMF (1.2 mL), H₂O (0.6 mL) were degassed and purged with N₂ three times, and then the mixture was stirred at 20° C. for 4 hrs under N₂ atmosphere. The reaction mixture was concentrated in vacuo to give a residue that was purified with prep. HPLC (water (0.225% FA)-ACN, YMC Triart C18 250*50 mm*7 μm). The residual aqueous solution was lyophilized to give (2S)-2-[[6-[3-(acetamidomethyl)phenoxy]pyridine-3-carbonyl]amino]-5,5-dimethyl-hexanoic acid (41 mg, 71% yield, 96% purity)) as a white solid.

¹H NMR (400 MHZ, CDCl₃) δ: 8.59 (br s, 1H), 8.13 (br d, J=7.75 Hz, 1H), 7.32-7.41 (m, 1H), 7.21 (br s, 1H), 7.14 (br d, J=7.25 Hz, 1H), 6.99-7.09 (m, 2H), 6.90 (br d, J=8.00 Hz, 1H), 6.51 (br s, 1H), 4.70 (br d, J=5.00 Hz, 1H), 4.44 (br s, 2H), 1.91-2.10 (m, 4H), 1.79 (br d, J=6.25 Hz, 1H), 1.14-1.39 (m, 3H), 0.87 (s, 9H).

### BF021

### (2S)-2-[[6-[[6-[2-[5-[(3aS,4S,6aR)-2-oxo-1,3,3a,4,6,6a-hexahydrothieno[3,4-d]imidazole-4-yl]pentanoylamino]ethylcarbamoyl]-1-naphthyl]oxy]pyridine-3-carbonyl]amino]-5,5-dimethyl-hexanoic acid

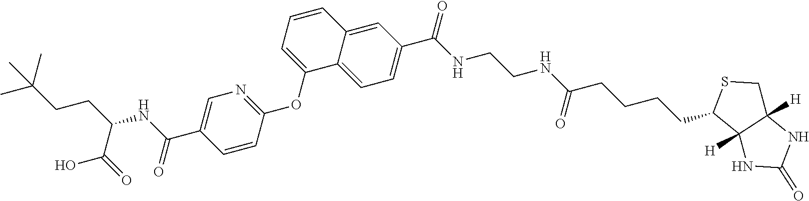 [SMILES: CC(C)(C)CC[C@H](NC(=O)c1ccc(Oc2cccc3cc(C(=O)NCCNC(=O)CCCC[C@@H]4SC[C@@H]5NC(=O)N[C@@H]54)ccc23)nc1)C(=O)O]

### Step 1: Synthesis of (2,5-dioxopyrrolidin-1-yl) 5-bromonaphthalene-2-carboxylate

To a solution of 5-bromonaphthalene-2-carboxylic acid (1.0 g, 3.98 mmol, 1 eq) in DMF (10 mL), 1-hydroxypyrrolidine-2,5-dione (1.15 g, 9.96 mmol, 2.5 eq) and EDCI (1.15 g, 5.97 mmol, 1.5 eq) were added and the mixture was degassed three times and stirred under N₂ at 20° C. for 3 h. The reaction was poured into H₂O (20 mL) and filtered. The filter cake was washed with H₂O (3×20 mL) and concentrated in vacuo to give (2,5-dioxopyrrolidin-1-yl) 5-bromonaphthalene-2-carboxylate (1.39 g, 100.00% yield, 100% purity) as a white solid.

¹H NMR (400 MHZ, DMSO) δ: ppm 8.93 (1H, d, J=0.8 Hz) 8.33 (1H, t, J=8.0 Hz) 8.22 (1H, d, J=0.8 Hz) 8.14 (1H, d, J=4.8 Hz) 7.62 (1H, t, J=8.0 Hz) 2.90 (4H, s)

### Step 2: Synthesis of N-[2-[5-[(3aS,4S,6aR)-2-oxo-1,3,3a,4,6,6a-hexahydrothieno[3,4-d]imidazol-4-yl]pentanoylamino]ethyl]-5-bromo-naphthalene-2-carboxamide

A mixture of 5-[(3aS,4S,6aR)-2-oxo-1,3,3a,4,6,6a-hexahydrothieno[3,4-d]imidazol-4-yl]-N-(2-aminoethyl) pentanamide (600 mg, 2.10 mmol, 1 eq), (2,5-dioxopyrrolidin-1-yl) 5-bromonaphthalene-2-carboxylate (729.38 mg, 2.10 mmol, 1 eq) and DIEA (812.28 mg, 6.29 mmol, 1.09 mL, 3 eq) in DMF (20 mL) was degassed and purged with N₂ three times, and then the mixture was stirred at 20° C. for 16 hrs under an N₂ atmosphere. The precipitate was filtered and dried under reduced pressure to give N-[2-[5-[(3aS,4S,6aR)-2-oxo-1,3,3a,4,6,6a-hexahydrothieno[3,4-d]imidazol-4-yl]pentanoylamino]ethyl]-5-bromo-naphthalene-2-carboxamide (1 g, crude) as a white solid, which was used directly without purification.

### Step 3: Synthesis of N-[2-[5-[(3aS,4S,6aR)-2-oxo-1,3,3a,4,6,6a-hexahydrothieno[3,4-d]imidazol-4-yl]pentanoylamino]ethyl]-5-hydroxy-naphthalene-2-carboxamide

To a mixture of N-[2-[5-[(3aS,4S,6aR)-2-oxo-1,3,3a,4,6,6a-hexahydrothieno[3,4-d]imidazol-4-yl]pentanoylamino]ethyl]-5-bromo-naphthalene-2-carboxamide (1 g, 1.93 mmol, 1 eq) in dioxane (30 mL), KOH (432.07 mg, 7.70 mmol, 4 eq) in H₂O (30 mL) was added. Then tBuXPhos Pd G3 (76.46 mg, 96.26 μμoλ, 0.05 eq) was added under N₂. The reaction was stirred at 110° C. under N₂ for 16 h. The reaction was extracted with EtOAc (50 mL×3). The aqueous layer was adjusted to pH 3-4 with 1M aq. HCl and extracted with EtOAc (150 mL×3). The organic layer was dried over Na₂SO₄ and concentrated to give N-[2-[5-[(3aS,4S,6aR)-2-oxo-1,3,3a, 4,6,6a-hexahydrothieno[3,4-d]imidazol-4-yl]pentanoylamino]ethyl]-5-hydroxy-naphthalene-2-carboxamide (220 mg, 385.49 μmol, 20.02% yield, 80% purity) as a yellow solid. The aqueous layer was concentrated and the crude product was triturated with MeOH (30 mL), filtered and concentrated to give N-[2-[5-[(3aS,4S,6aR)-2-oxo-1,3,3a,4,6,6a-hexahydrothieno[3,4-d]imidazol-4-yl]pentanoylamino]ethyl]-5-hydroxy-naphthalene-2-carboxamide (350 mg, crude) as a light-red solid.

### Step 4: Synthesis of 6-[[6-[2-[5-[(3aS, 4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylate

To a solution of N-[2-[5-[(3aS,4S,6aR)-2-oxo-1,3,3a,4,6,6a-hexahydrothieno[3,4-d]imidazol-4-yl]pentanoylamino]ethyl]-5-hydroxy-naphthalene-2-carboxamide (120 mg, 262.84 μmol, 1 eq) and Cs₂CO₃ (128.46 mg, 394.26 μmol, 1.5 eq) in DMF (3 mL) was added methyl 6-fluoropyridine-3-carboxylate (44.85 mg, 289.12 μmol, 1.1 eq). The reaction was stirred at 25° C. for 16 hr. H₂O (10 mL) was added to the solution and the solution was extracted with EtOAc (50 mL×8). The organic layer was dried over Na₂SO₄ and concentrated. The crude product was triturated with MeOH (5 mL), filtered and dried under reduced pressure to give methyl 6-[[6-[2-[5-[(3aS, 4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylate (160 mg, 47.47% yield, 92.28% purity) as an off-white solid.

LCMS (ESI) [M+H]⁺ m/z: calc'd 591.28, found 592.3

### Step 5: Synthesis of 6-[[6-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylic acid

To a solution of methyl 6-[[6-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylate (110 mg, 185.91 μmol, 1 eq) in THF (3 mL), MeOH (2 mL) and H₂O (1 mL) was added LiOH·H₂O (15.60 mg, 371.82 μmol, 2 eq). The reaction was stirred at 25° C. for 2 hr. The MeOH and THF were removed in vacuo and the aqueous layer was adjusted to pH 4-5 with 1N aq. HCl and extracted with EtOAc (20 mL×2). The organic layer was dried over Na₂SO₄ and concentrated to give 6-[[6-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylic acid (160 mg, 88.29% yield, 94.5% purity, HCl salt) as a brown solid, which was used directly without purification.

LCMS (ESI) [M+Na]⁺ m/z: calc'd 577.2, found 578.1

¹H NMR (400 MHZ, DMSO-d₆) δ: ppm 8.81 (1H, br t, J=5.3 Hz) 8.58 (2H, d, J=1.8 Hz) 8.34 (1H, dd, J=8.7, 2.4 Hz) 8.09 (1H, br t, J=5.0 Hz) 7.91-7.99 (2H, m) 7.83 (1H, d, J=8.8 Hz) 7.65 (1H, t, J=7.9 Hz) 7.46 (1H, d, J=7.5 Hz) 7.29 (1H, d, J=8.5 Hz) 6.42 (1H, br d, J=2.0 Hz) 4.23 (1H, dd, J=7.7, 5.1 Hz) 4.04 (1H, dd, J=7.7, 4.4 Hz) 3.36 (2H, br d, J=6.0 Hz) 3.24-3.29 (2H, m) 2.97-3.03 (1H, m) 2.71-2.77 (1H, m) 2.08 (2H, br t, J=7.4 Hz) 1.38-1.58 (4H, m) 1.21-1.35 (3H, m)

### Step 6: Synthesis of (2,5-dioxopyrrolidin-1-yl) 6-[[6-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylate

To a solution of 6-[[6-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylic acid (110 mg, 190.43 μmol, 1 eq) and EDCI (54.76 mg, 285.64 μmol, 1.5 eq) in DMF (5 mL) was added 1-hydroxypyrrolidine-2,5-dione (54.79 mg, 476.07 μmol, 2.5 eq). The reaction was stirred at 25° C. for 3 hr. The reaction was concentrated to give (2,5-dioxopyrrolidin-1-yl) 6-[[6-[2-[5-[(3aS,4S,6aR)-2-oxo-1,3,3a, 4,6, 6a-hexahydrothieno[3,4-d]imidazol-4-yl]pentanoylamino]ethylcarbamoyl]-1-naphthyl]oxy]pyridine-3-carboxylate (128.4 mg, crude), as a yellow oil which was used directly without purification.

### Step 7: (2S)-2-[[6-[[6-[2-[5-[(3aS,4S,6aR)-2-oxo-1,3,3a,4,6,6a-hexahydrothieno[3,4-d]imidazole-4-yl]pentanoylamino]ethylcarbamoyl]-1-naphthyl]oxy]pyridine-3-carbonyl]amino]-5,5-dimethyl-hexanoic acid

A mixture of (2,5-dioxopyrrolidin-1-yl) 6-[[6-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylate (128.4 mg, 190.30μμ ολ, 1 eq), (2S)-2-amino-5,5-dimethyl-hexanoic acid (30.30 mg, 190.30 μmol, 1 eq) and DIEA (36.89 mg, 285.45 μmol, 49.72 μL, 1.5 eq) in DCM (0.5 mL), DMF (3.0 mL) and H₂O (1.5 mL) were degassed and purged with N₂ three times, and then the mixture was stirred at 25° C. for 3 hrs under an N₂ atmosphere. The reaction was concentrated and the residue was purified by Prep. HPLC (column: Boston Green ODS 150*30 mm*5□m; mobile phase: [water (0.05% HCl)-ACN]; B %: 21%-61%,9 min) to give (2S)-2-[[6-[[6-[2-[5-[(3aS,4S,6aR)-2-oxo-1,3,3a,4,6,6a-hexahydrothieno[3,4-d]imidazole-4-yl]pentanoylamino]ethylcarbamoyl]-1-naphthyl]oxy]pyridine-3-carbonyl]amino]-5,5-dimethyl-hexanoic acid (18 mg, 13.16% yield, 100% purity) as a white solid.

LCMS (ESI) [M+H]⁺ m/z: calc'd 718.3, found 719.1

¹H NMR (400 MHZ, DMSO-d₆) δ: ppm 8.60-8.71 (2H, m) 8.47-8.55 (2H, m) 8.34 (1H, dd, J=8.6, 2.5 Hz) 7.81-7.99 (4H, m) 7.64 (1H, t, J=7.9 Hz) 7.43 (1H, dd, J=7.5, 0.8 Hz) 7.25-7.30 (1H, m) 6.39 (2H, br s) 4.30 (1H, td, J=8.5, 5.2 Hz) 4.24 (1H, dd, J=7.7, 4.5 Hz) 4.04 (1H, dd, J=7.6, 4.5 Hz) 3.36 (2H, br d, J=6.0 Hz) 3.25 (2H, q, J=6.0 Hz) 2.96-3.04 (1H, m) 2.74 (1H, dd, J=12.4, 5.1 Hz) 2.52-2.58 (1H, m) 2.07 (2H, t, J=7.3 Hz) 1.67-1.85 (2H, m) 1.40-1.61 (4H, m) 1.19-1.36 (4H, m) 0.83-0.87 (9H, m)

### BF027

### (2S)-2-[[6-[[5-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carbonyl]amino]-5,5-dimethyl-hexanoic acid

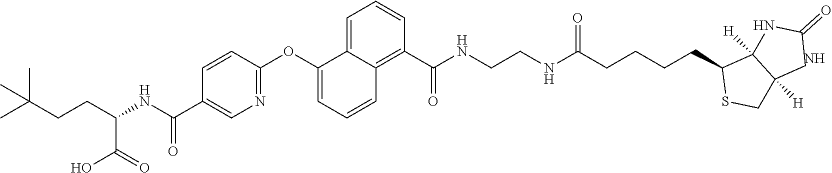

### Step 1: Synthesis of (2,5-dioxopyrrolidin-1-yl) 5-bromonaphthalene-1-carboxylate

To a solution of 5-bromonaphthalene-1-carboxylic acid (1 g, 3.98 mmol, 1 eq) in DMF (10 mL) was added 1-hydroxypyrrolidine-2,5-dione (1.15 g, 9.96 mmol, 2.5 eq) and EDCI (1.15 g, 5.97 mmol, 1.5 eq) and the mixture was degassed three times and stirred under N₂ at 20° C. for 3 h. The reaction was poured into H₂O (20 mL) and filtered. The filter cake was washed with H₂O (3×20 mL) and concentrated in vacuo to give (2,5-dioxopyrrolidin-1-yl) 5-bromonaphthalene-1-carboxylate (1.39 g, 100.00% yield) as a white solid.

¹H NMR (400 MHZ, DMSO-d₆) δ: ppm 8.64-8.60 (2H, m) 8.43 (1H, m) 8.10 (1H, m), 7.90 (1H, m), 7.70-7.65 (1H, m), 2.95 (4H, s).

### Step 2: N-[2-[5-[(3aS,4S,6aR)-2-oxo-1,3,3a,4,6,6a-hexahydrothieno[3,4-d]imidazol-4-yl]pentanoylamino]ethyl]-5-bromo-naphthalene-1-carboxamide

To a solution of 5-[(3aS,4S,6aR)-2-oxo-1,3,3a,4,6,6a-hexahydrothieno[3,4-d]imidazol-4-yl]-N-(2-aminoethyl) pentanamide (1 g, 3.49 mmol, 1 eq) in DMF (60 mL) was added DIPEA (902.54 mg, 6.98 mmol, 1.22 mL, 2 eq) and (2,5-dioxopyrrolidin-1-yl)-5-bromonaphthalene-1-carboxylate (1.22 g, 3.49 mmol, 1 eq). The mixture was stirred at 20° C. for 16 hr. The reaction was filtered and the filter cake was washed with H₂O (3×20 mL) and concentrated in vacuo to give N-[2-[5-[(3aS,4S,6aR)-2-oxo-1,3,3a,4,6,6a-hexahydrothieno[3,4-d]imidazol-4-yl]pentanoylamino]ethyl]-5-bromo-naphthalene-1-carboxamide (1.38 g, 76.08% yield) as a white solid.

LCMS (ESI) [M+H]⁺ m/z: calc'd 520.5, found 521.2

### Step 3: N-[2-[5-[(3aS,4S,6aR)-2-oxo-1,3,3a,4,6,6a-hexahydrothieno[3,4-d]imidazol-4-yl]pentanoylamino]ethyl]-5-hydroxy-naphthalene-1-carboxamide

To a solution of N-[2-[5-[(3aS,4S,6aR)-2-oxo-1,3,3a,4,6,6a-hexahydrothieno[3,4-d]imidazol-4-yl]pentanoylamino]ethyl]-5-bromo-naphthalene-1-carboxamide (1 g, 1.93 mmol, 1 eq) in dioxane (20 mL) and H₂O (20 mL) was added tBuXPhos Pd G3 (76.46 mg, 96.26 □mol, 0.05 eq) and KOH (432.04 mg, 7.70 mmol, 4 eq). The mixture was stirred at 110° C. for 16 hr. The reaction mixture was concentrated under reduced pressure and was washed with MTBE (30 mL×2). The solid was acidified to pH 5 with 1M HCl, collected and dried in vacuo to give N-[2-[5-[(3aS,4S,6aR)-2-oxo-1,3,3a,4,6,6a-hexahydrothieno[3,4-d]imidazol-4-yl]pentanoylamino]ethyl]-5-hydroxy-naphthalene-1-carboxamide (800 mg, crude) as a brown solid, which was used directly without further purification.

LCMS (ESI) [M+H]⁺ m/z: calc'd 457.5, found 457.3

### Step 4: Methyl-6-[[5-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylate

To a solution of N-[2-[5-[(3aS,4S,6aR)-2-oxo-1,3,3a,4,6,6a-hexahydrothieno[3,4-d]imidazol-4-yl]pentanoylamino]ethyl]-5-hydroxy-naphthalene-1-carboxamide (800 mg, 1.75 mmol, 1 eq) in DMF (10 mL) was added methyl 6-fluoropyridine-3-carboxylate (271.82 mg, 1.75 mmol, 1 eq) and Cs₂CO₃ (1.14 g, 3.50 mmol, 2 eq). The reaction mixture was stirred at 20° C. for 16 hrs. The reaction mixture was washed with H₂O (40 mL) and extracted with EA (40 mL×3). The organic layer was dried with Na₂SO₄, filtered and concentrated under reduced pressure to give methyl 6-[[5-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylate (800 mg, 38.58% yield) as a grey solid, which was used without purification.

LCMS (ESI) [M+H]⁺ m/z: calc'd 591.7, found 592.1

### Step 5: 6-[[5-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylic acid

To a solution of methyl 6-[[5-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylate (800 mg, 1.35 mmol, 1 eq) in a mixture of THF (12 mL) and H₂O (4 mL) was added LiOH·H₂O (113.48 mg, 2.70 mmol, 2 eq). The mixture was stirred at 25° C. for 4 hr. The mixture was acidified with HCl (1M) to pH 5 and concentrated under reduced pressure to give 6-[[5-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylic acid (100 mg, crude) as a yellow solid.

LCMS (ESI) [M+Na]⁺ m/z: calc'd 577.2, found 578.3

### Step 6: (2,5-dioxopyrrolidin-1-yl)-6-[[5-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylate

To a solution of 6-[5-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylic acid (100 mg, 173.12 μmol, 1 eq) in DMF (2 mL) was added 1-hydroxypyrrolidine-2,5-dione (49.81 mg, 432.79 μmol, 2.5 eq) and EDC (49.78 mg, 259.67 μmol, 1.5 eq) and the mixture was degassed three times and stirred under N₂ at 20° C. for 4 h. The reaction mixture was concentrated under reduced pressure to give (2,5-dioxopyrrolidin-1-yl) 6-[[5-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylate (100 mg, crude) as a pale yellow gum.

### Step 7: (2S)-2-[[6-[[5-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carbonyl]amino]-5,5-dimethyl-hexanoic acid

A mixture of (2,5-dioxopyrrolidin-1-yl)-6-[[5-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carboxylate (100 mg, 148.21 μmol, 1 eq), (2S)-2-amino-5,5-dimethyl-hexanoic acid (23.60 mg, 148.21 μmol, 1 eq) and DIEA (28.73 mg, 222.31 μμoλ, 38.72 uL, 1.5 eq) in DCM (0.4 mL), DMF (2.4 mL) and H₂O (1.2 mL) was degassed and purged with N₂ three times, and then the mixture was stirred at 20° C. for 4 hrs under an N₂ atmosphere. The reaction mixture was concentrated under reduced pressure and the residue was purified by prep. HPLC (HCl condition) to give (2S)-2-[[6-[[5-[2-[5-[(3aS,4S,6aR)-2-oxo-1,3,3a,4,6,6a-hexahydrothieno[3,4-d]imidazol-4-yl]pentanoylamino]ethylcarbamoyl]-1-naphthyl]oxy]pyridine-3-carbonyl]amino]-5,5-dimethyl-hexanoic acid (5 mg, 4.53% yield, 96.6% purity) as a white solid.

¹H NMR (400 MHZ, DMSO-d₆) δ: ppm 8.65-8.70 (1H, m) 8.58-8.64 (1H, m) 8.55 (1H, m) 8.33 (1H, dd, J=8.8, 2.4 Hz) 8.13 (1H, d, J=8.4 Hz) 7.90 (1H, m) 7.88 (1H, d, J=8. Hz) 7.61-7.68 (2H, m) 7.52-7.56 (1H, m) 7.38 (1H, d, J=6.8 Hz) 7.26 (1H, d, J=8.4 Hz) 6.40 (1H, br s) 6.33 (1H, br s) 4.24-4.31 (2H, m) 4.05-4.09 (1H, m) 3.25-3.35 (4H, m) 3.04-3.08 (1H, m) 2.75 (1H, dd, J=12.4, 5.1 Hz) 2.07 (2H, t, J=7.3 Hz) 1.32-1.81 (5H, m) 1.15-1.30 (5H, m) 0.85 (9H, s)

LCMS (ESI) [M+H]⁺ m/z: calc'd 718.31, found 719.2

### BF022

### (S)-5,5-dimethyl-2-(6-(3-(2-(2-(2-(5-((3aS,4S,6aR)-2-oxohexahydro-1H-thieno[3,4-d]imidazol-4-yl)pentanamido)ethoxy)ethoxy)ethoxy)phenoxy) nicotinamido) hexanoic acid

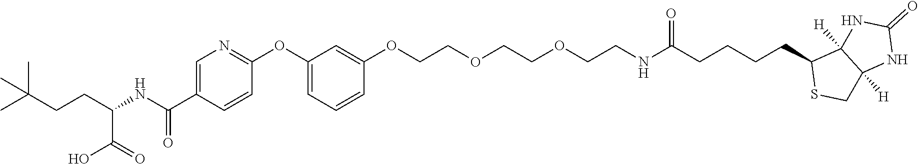 [SMILES: CC(C)(C)CC[C@H](NC(=O)c1ccc(Oc2cccc(OCCOCCOCCNC(=O)CCCC[C@@H]3SC[C@@H]4NC(=O)N[C@@H]43)c2)nc1)C(=O)O]

### Step 1:2-[2-[2-(Tert-butoxycarbonylamino)ethoxy]ethoxy]ethyl 4-methylbenzenesulfonate

To a solution of tert-butyl N-[2-[2-(2-hydroxyethoxy)ethoxy]ethyl]carbamate (1.5 g, 6.02 mmol, 1 eq) in DCM (30 mL) was added p-toluenesulfonyl chloride (2.29 g, 12.03 mmol, 2.0 eq) and Et₃N (1.83 g, 18.05 mmol, 2.51 mL, 3.0 eq). The mixture was stirred at 20° C. for 16 hr. The mixture was poured into water (100 mL) and the aqueous phase was extracted with DCM (20 mL×3). The combined organic phase was washed with brine (20 mL), dried with anhydrous Na₂SO₄, filtered and concentrated in vacuo. The residue was purified by silica gel chromatography (column height: 250 mm, diameter: 100 mm, 100-200 mesh silica gel, petroleum ether/ethyl acetate=20/1 to 5/1) to afford 2-[2-[2-(tert-butoxycarbonylamino)ethoxy]ethoxy]ethyl 4-methylbenzenesulfonate (2.3 g, 94.74% yield) as a brown oil.

LCMS (ESI) [M+Na]⁺ m/z: calc'd 426.1, found 426.1

¹H NMR (400 MHZ, CHLOROFORM-d) □: ppm 7.81 (2H, d, J=8.3 Hz) 7.35 (2H, d, J=8.0 Hz) 4.94 (1H, br s) 4.13-4.23 (2H, m) 3.68-3.73 (2H, m) 3.48-3.61 (6H, m) 3.30 (2H, br s) 2.36-2.55 (3H, m) 1.44 (9H, s)

### Step 2: Tert-butyl (2-(2-(2-(3-hydroxyphenoxy)ethoxy)ethoxy)ethyl) carbamate

To a solution of 2-[2-[2-(tert-butoxycarbonylamino)ethoxy]ethoxy]ethyl 4-methylbenzenesulfonate (2.0 g, 4.96 mmol, 1.0 eq) and (3-hydroxyphenyl) acetate (904.99 mg, 5.95 mmol, 1.2 eq) in DMF (40 mL) was added K₂CO₃ (2.06 g, 14.87 mmol, 3.0 eq). The mixture was stirred at 100° C. for 16 hr. The mixture was poured into water (100 mL) and extracted with EtOAc (50 mL×2). The combined organic phase was washed with brine (50 mL), dried with anhydrous Na₂SO₄, filtered and concentrated in vacuo. The residue was purified by silica gel chromatography (column height: 250 mm, diameter: 100 mm, 100-200 mesh silica gel, petroleum ether/ethyl acetate=10/1 to 1/1) to afford tert-butylN-[2-[2-[2-(3-hydroxyphenoxy)ethoxy]ethoxy]ethyl]carbamate (1.0 g, 59.1% yield) as a light-yellow oil.

LCMS (ESI) [M+Na]⁺ m/z: calc'd 341.2, found 364.2

### Step 3: Methyl 6-[3-[2-[2-[2-(Tert-butoxycarbonylamino)ethoxy]ethoxy]ethoxy]phenoxy]pyridine-3-carboxylate

To a mixture of tert-butyl N-[2-[2-[2-(3-hydroxyphenoxy)ethoxy]ethoxy]ethyl]carbamate (1.0 g, 2.93 mmol, 1 eq) and methyl 6-fluoropyridine-3-carboxylate (545.26 mg, 3.51 mmol, 1.2 eq) in DMF (20 mL) was added Cs₂CO₃ (2.86 g, 8.79 mmol, 3.0 eq). The mixture was stirred at 80° C. for 16 hours. The mixture was cooled to 20° C. and poured into water (100 mL). The aqueous phase was extracted with ethyl acetate (60 mL×3). The combined organic phase was washed with brine (60 mL), dried with anhydrous Na₂SO₄, filtered and concentrated in vacuo. The residue was purified by silica gel chromatography (column height: 250 mm, diameter: 100 mm, 100-200 mesh silica gel, petroleum ether/ethyl acetate=10/1 to 2/1) to afford methyl 6-[3-[2-[2-[2-(tert-butoxycarbonylamino)ethoxy]ethoxy]ethoxy]phenoxy]pyridine-3-carboxylate (900 mg, 64.4% yield) as a light-yellow oil.

LCMS (ESI) [M+Na]⁺ m/z: calc'd 477.2, found 499.3

### Step 4: Methyl 6-(3-(2-(2-(2-aminoethoxy)ethoxy)ethoxy)phenoxy) nicotinate

To a mixture of methyl 6-[3-[2-[2-[2-(tert-butoxycarbonylamino)ethoxy]ethoxy]ethoxy]phenoxy]pyridine-3-carboxylate (1.0 g, 2.10 mmol, 1 eq) in DCM (20 mL) was added TFA (11.96 g, 104.93 mmol, 7.77 mL, 50 eq). The mixture was stirred at 20° C. for 3 hours. The mixture was concentrated in vacuum and dissolved in DCM (200 mL), basified with saturated aqueous NaHCO₃ until pH 8, then extracted with DCM (3×50 mL). The combined organic layers were washed with brine (50 mL), dried over anhydrous Na₂SO₄, filtered and concentrated in vacuo to afford methyl 6-[3-[2-[2-(2-aminoethoxy)ethoxy]ethoxy]phenoxy]pyridine-3-carboxylate (900 mg, 94.9% yield) as a yellow oil.

LCMS (ESI) [M+H]⁺ m/z: calc'd 377.2, found 377.1

### Step 5: Methyl 6-(3-(2-(2-(2-(5-((3aS,4S,6aR)-2-oxohexahydro-1H-thieno[3,4-d]imidazol-4-yl)pentanamido)ethoxy)ethoxy)ethoxy)phenoxy) nicotinate

To a mixture of 5-[(3aS,4S,6aR)-2-oxo-1,3,3a,4,6,6a-hexahydrothieno[3,4-d]imidazol-4-yl] pentanoic acid (493.29 mg, 2.02 mmol, 0.95 eq) in DMF (15 mL) was added HATU (1.21 g, 3.19 mmol, 1.5 eq) and DIEA (824.07 mg, 6.38 mmol, 1.11 mL, 3.0 eq) under N₂. The mixture was stirred at 20° C. for 30 min, then methyl 6-[3-[2-[2-(2-aminoethoxy)ethoxy]ethoxy]phenoxy]pyridine-3-carboxylate (800 mg, 2.13 mmol, 1 eq) was added. The reaction was stirred for 1 hour then the mixture was poured into water (100 mL). The aqueous phase was extracted with ethyl acetate (50 mL×3). The combined organic phase was washed with brine (50 mL), dried with anhydrous Na₂SO₄, filtered and concentrated in vacuo. The residue was purified by silica gel chromatography (column height: 250 mm, diameter: 100 mm, 100-200 mesh silica gel, MeOH/ethyl acetate=0/1 to 1/10) to afford methyl 6-[3-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]phenoxy]pyridine-3-carboxylate (900 mg, 70.3% yield) as a yellow solid.

LCMS (ESI) [M+H]⁺ m/z: calc'd 603.2, found 603.4

1H NMR (400 MHZ, CHLOROFORM-d) δ: ppm 8.82 (1H, d, J=1.8 Hz) 8.28 (1H, dd, J=8.5, 2.3 Hz) 7.33 (1H, t, J=8.2 Hz) 6.94 (1H, d, J=8.3 Hz) 6.83 (1H, d, J=1.8 Hz) 6.69-6.80 (2H, m) 6.63 (1H, br t, J=5.4 Hz) 6.19 (1H, br s) 4.49 (1H, dd, J=7.7, 4.6 Hz) 4.29 (1H, dd, J=7.7, 4.6 Hz) 4.12-4.16 (3H, m) 3.93 (3H, s) 3.82-3.88 (2H, m) 3.69-3.74 (2H, m) 3.63-3.68 (2H, m) 3.54-3.61 (2H, m) 3.40-3.47 (2H, m) 3.08-3.14 (1H, m) 2.89 (1H, dd, J=12.8, 5.0 Hz) 2.73 (1H, d, J=12.8 Hz) 2.21 (2H, td, J=7.3, 2.5 Hz) 1.99 (1H, br s) 1.60-1.75 (4H, m) 1.34-1.50 (4H, m)

### Step 6: 6-(3-(2-(2-(2-(5-((3aS,4S,6aR)-2-oxohexahydro-1H-thieno[3,4-d]imidazol-4-yl)pentanamido)ethoxy)ethoxy)ethoxy)phenoxy) nicotinic acid

To a mixture of methyl 6-[3-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]phenoxy]pyridine-3-carboxylate (900 mg, 1.49 mmol, 1 eq) in THF (10 mL), MeOH (5 mL), H₂O (5 mL) was added LiOH·H₂O (187.99 mg, 4.48 mmol, 3.0 eq). The mixture was stirred at 20° C. for 2 hours. The mixture was concentrated in vacuo to remove MeOH then the aqueous layer was acidified with aqueous 1M HCl until pH 1˜2, and extracted with EtOAc (3×50 mL). The combined organic layers were washed with brine (50 mL), dried over anhydrous Na₂SO₄, filtered and concentrated in vacuo. The residue was purified by silica gel chromatography (column height: 250 mm, diameter: 100 mm, 100-200 mesh silica gel, MeOH/ethyl acetate=0/1 to 1/10) to afford 6-[3-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]phenoxy]pyridine-3-carboxylic acid (450 mg, 51.2% yield) as a white gum.

LCMS (ESI) [M+H]⁺ m/z: calc'd 589.2 found 589.1

### Step 7:2,5-dioxopyrrolidin-1-yl-6-(3-(2-(2-(2-(5-((3aS,4S,6aR)-2-oxohexahydro-1H-thieno[3,4-d]imidazol-4-yl)pentanamido)ethoxy)ethoxy)ethoxy)phenoxy) nicotinate

To a mixture of 6-[3-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]phenoxy]pyridine-3-carboxylic acid (500 mg, 849.37 μmol, 1 eq) and 1-hydroxypyrrolidine-2,5-dione (146.63 mg, 1.27 mmol, 1.5 eq) in DMF (10 mL) was added EDC (244.24 mg, 1.27 mmol, 1.5 eq) under N₂. The mixture was stirred at 20° C. for 2 hours. The mixture was poured into water (50 mL) and saturated aqueous NaHCO₃ (10 mL). The aqueous phase was extracted with ethyl acetate (30 mL×3). The combined organic phase was washed with brine (30 mL), dried with anhydrous Na₂SO₄, filtered and concentrated in vacuo to afford (2,5-dioxopyrrolidin-1-yl) 6-[3-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]phenoxy]pyridine-3-carboxylate (500 mg, 85.8% yield) as a light-yellow oil, which was used directly without further purification.

LCMS (ESI) [M+H]⁺ m/z: calc'd 686.3, found 686.5

### Step 8: (S)-5,5-dimethyl-2-(6-(3-(2-(2-(2-(5-((3aS,4S,6aR)-2-oxohexahydro-1H-thieno[3,4-d]imidazol-4-yl)pentanamido)ethoxy)ethoxy)ethoxy)phenoxy) nicotinamido) hexanoic acid

To a mixture of (2,5-dioxopyrrolidin-1-yl) 6-[3-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]phenoxy]pyridine-3-carboxylate (300 mg, 437.48 μmol, 1 eq) and (2S)-2-amino-5,5-dimethyl-hexanoic acid (69.66 mg, 437.48 μmol, 1 eq) in DMF (10 mL) was added DIEA (169.62 mg, 1.31 mmol, 228.60 μL, 3.0 eq) under N₂. The mixture was stirred at 20° C. for 16 hours. The mixture was poured into water (100 mL) and the aqueous phase was extracted with ethyl acetate (50 mL×3). The combined organic phase was washed with brine (50 mL), dried with anhydrous Na₂SO₄, filtered and concentrated in vacuo. The residue was purified by prep-HPLC (column: Boston Green ODS 150*30 mm*5 um; mobile phase: [water (0.05% HCl)-ACN]; B %: 22%-62%, 9 min) to afford (2S)-2-[[6-[3-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]phenoxy]pyridine-3-carbonyl]amino]-5,5-dimethyl-hexanoic acid (90 mg, 26.84% yield, HCl salt) as a white solid.

LCMS (ESI) [M+H]⁺ m/z: calc'd 730.3, found 730.3

¹H NMR (400 MHZ, DMSO-d₆) δ: ppm 8.69 (1H, d, J=7.8 Hz) 8.64 (1H, d, J=2.3 Hz) 8.28 (1H, dd, J=8.5, 2.5 Hz) 7.83 (1H, t, J=5.4 Hz) 7.33 (1H, t, J=8.2 Hz) 7.09 (1H, d, J=8.5 Hz) 6.84 (1H, dd, J=7.9, 2.1 Hz) 6.70-6.80 (2H, m) 6.41 (1H, br s) 4.26-4.38 (2H, m) 4.07-4.17 (3H, m) 3.71-3.77 (2H, m) 3.56-3.61 (2H, m) 3.50-3.54 (3H, m) 3.18 (2H, q, J=5.9 Hz) 3.06-3.11 (1H, m) 2.81 (1H, dd, J=12.4, 5.1 Hz) 2.51-2.56 (3H, m) 2.01-2.09 (3H, m) 1.68-1.83 (2H, m) 1.55-1.65 (1H, m) 1.39-1.53 (3H, m) 1.20-1.35 (4H, m) 0.87 (9H, s)

¹H NMR (400 MHZ, CHLOROFORM-d) δ: ppm 8.67 (1H, s) 8.27 (1H, br d, J=7.1 Hz) 7.78 (1H, br d, J=7.4 Hz) 7.30-7.38 (2H, m) 6.93-7.05 (1H, m) 6.80 (2H, br t, J=7.8 Hz) 6.74 (1H, br s) 6.41 (1H, br s) 4.73 (1H, br d, J=3.9 Hz) 4.54 (1H, br s) 4.36 (1H, br s) 4.13-4.21 (2H, m) 3.85 (2H, br s) 3.58-3.75 (4H, m) 3.50-3.57 (2H, m) 3.36 (2H, br s) 3.19 (1H, br s) 2.94 (1H, br d, J=9.8 Hz) 2.74 (1H, br d, J=12.6 Hz) 2.12 (3H, br d, J=7.1 Hz) 1.96-2.05 (5H, m) 1.32-1.45 (5H, m) 0.89 (9H, s)

### BF025

### 2-[[6-[3-[2-[2-[2-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]ethoxy]ethoxy]ethoxy phenoxy]pyridine-3-carbonyl]amino]-5,5-dimethyl-hexanoic acid

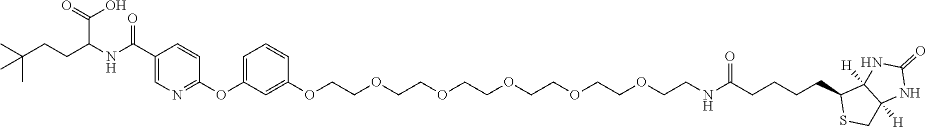 [SMILES: CC(C)(C)CCC(NC(=O)c1ccc(Oc2cccc(OCCOCCOCCOCCOCCOCCNC(=O)CCCC[C@@H]3SC[C@@H]4NC(=O)N[C@@H]43)c2)nc1)C(=O)O]

### Step 1:2-[2-[2-[2-[2-[2-(tert-butoxycarbonylamino)ethoxy]ethoxy]ethoxy]ethoxy]ethoxy]ethyl 4-methylbenzenesulfonate

tert-butyl N-[2-[2-[2-[2-[2-(2-hydroxyethoxy)ethoxy]ethoxy]ethoxy]ethoxy]ethyl]carbamate (100 mg, 262.15 μμoλ, 1 eq) was dissolved in THF (1 mL). NaOH (20.97 mg, 524.30 μmol, 2 eq) dissolved in H₂O (0.2 mL) was added. The mixture was cooled to 0° C. and 4-methylbenzenesulfonyl chloride (59.97 mg, 314.58 μmol, 1.2 eq) dissolved in THF (1 mL) was added dropwise. The reaction mixture was allowed to warm to 20° C. and was stirred 16 hr. TLC (KMnO₄, DCM:MeOH=10:1) showed the reaction was completed. THF was evaporated, 50 mL H₂O was added, followed by two extractions with 5 mL CH₂Cl₂ and washing with 5 mL brine. The product was dried over MgSO₄ and concentrated. The residue was purified by silica gel chromatography eluted with (DCM:MeOH=50:1 to 10:1) to give 2-[2-[2-[2-[2-[2-(tert-butoxycarbonylamino)ethoxy]ethoxy]ethoxy]ethoxy]ethoxy]ethyl 4-methylbenzenesulfonate (100 mg, 71.21% yield, 100% purity) as a colourless oil.

¹H NMR (400 MHZ, CHLOROFORM-d) δ: ppm 7.82 (2H, d, J=8.1 Hz) 7.36 (2H, d, J=8.0 Hz) 5.06 (1H, br s) 4.11-4.26 (2H, m) 3.50-3.80 (21H, m) 3.33 (2H, br s) 2.47 (3H, s) 1.46 (9H, s)

### Step 2: Tert-butyl N-[2-[2-[2-[2-[2-[2-(3-hydroxyphenoxy)ethoxy]ethoxy]ethoxy]ethoxy]ethoxy]ethyl]carbamate

To a solution of 2-[2-[2-[2-[2-[2-(tert-butoxycarbonylamino)ethoxy]ethoxy]ethoxy]ethoxy]ethoxy]ethyl 4-methylbenzenesulfonate (800 mg, 1.49 mmol, 1 eq) and (3-hydroxyphenyl) acetate (272.68 mg, 1.79 mmol, 1.2 eq) in DMF (10 mL) was added Cs₂CO₃ (1.46 g, 4.48 mmol, 3 eq). The reaction was stirred at 80° C. for 16 h under N₂. TLC (DCM:MeOH=10:1) showed the reaction was completed. The reaction mixture was concentrated to the residue, to which was added H₂O (20 mL), and extracted with EtOAc (20 mL×3). The combined organic layer was washed with brine (25 mL), dried over anhydrous Na₂SO₄, filtered and concentrated. The crude product was purified by silica gel chromatography eluted with (PE:EtOAc=100:1 to 1:3) to give tert-butyl N-[2-[2-[2-[2-[2-[2-(3-hydroxyphenoxy)ethoxy]ethoxy]ethoxy]ethoxy]ethoxy]ethyl]carbamate (360 mg, 45.56% yield, 89.5% purity) as a yellow oil.

LCMS (ESI) [M+Na]⁺ m/z: calc'd 496.2, found 496.2

### Step 3: Methyl 6-[3-[2-[2-[2-[2-[2-[2-(tert-butoxycarbonylamino)ethoxy]ethoxy]ethoxy]ethoxy]ethoxy]ethoxy]phenoxy]pyridine-3-carboxylate

To a solution of tert-butyl N-[2-[2-[2-[2-[2-[2-(3-hydroxyphenoxy)ethoxy]ethoxy]ethoxy]ethoxy]ethoxy]ethyl]carbamate (360 mg, 760.21 μmol, 1 eq) and methyl 6-fluoropyridine-3-carboxylate (141.51 mg, 912.25 μmol, 1.2 eq) in DMF (8 mL) was added Cs₂CO₃ (743.07 mg, 2.28 mmol, 3 eq). The reaction was stirred at 80° C. for 20 h. LCMS showed the reaction was completed. The reaction mixture was concentrated to the residue, to which was added H₂O (15 mL) and extracted with EtOAc (20 mL×3). The combined organic layer was washed with brine (20 mL), dried over anhydrous Na₂SO₄, filtered and concentrated. The crude product was purified by silica gel chromatography eluted with (DCM:MeOH=100:1 to 10:1) to give methyl 6-[3-[2-[2-[2-[2-[2-[2-(tert-butoxycarbonylamino)ethoxy]ethoxy]ethoxy]ethoxy]ethoxy]ethoxy]phenoxy]pyridine-3-carboxylate (247 mg, 48.03% yield, 89.98% purity) as a yellow oil.

LCMS (ESI) [M+Na]⁺ m/z: calc'd 631.3, found 631.1

### Step 4: Methyl 6-[3-[2-[2-[2-[2-[2-(2-aminoethoxy) ethoxy]ethoxy]ethoxy]ethoxy]ethoxy]phenoxy]pyridine-3-carboxylate

To a solution of methyl 6-[3-[2-[2-[2-[2-[2-[2-(tert-butoxycarbonylamino)ethoxy]ethoxy]ethoxy]ethoxy]ethoxy]ethoxy]phenoxy]pyridine-3-carboxylate (247 mg, 405.80 μmol, 1 eq) in DCM (10 mL) was added TFA (925.41 mg, 8.12 mmol, 600.91 μL, 20 eq). The reaction was stirred at 20° C. for 20 h. TLC showed the reaction was completed. The reaction was basified with saturated aqueous NaHCO₃ until pH 8, then extracted with DCM (3×10 mL). The combined organic layers were washed with brine (15 mL), dried over anhydrous Na₂SO₄, filtered and concentrated in vacuo to give methyl 6-[3-[2-[2-[2-[2-[2-(2-aminoethoxy)ethoxy]ethoxy]ethoxy]ethoxy]ethoxy]phenoxy]pyridine-3-carboxylate (206 mg, 84.95% yield, 85.1% purity) as a yellow oil, which was used directly without further purification.

LCMS (ESI) [M+Na]⁺ m/z: calc'd 509.2, found 509.3

### Step 5: Methyl 6-[3-[2-[2-[2-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]ethoxy]ethoxy]ethoxy]phenoxy]pyridine-3-carboxylate

To a solution of 5-[(3aS,4S,6aR)-2-oxo-1,3,3a,4,6,6a-hexahydrothieno[3,4-d]imidazol-4-yl]pentanoic acid (74.94 mg, 306.75 μmol, 1 eq) in DMF (1 mL) was added HATU (174.95 mg, 460.12 μmol, 1.5 eq) and DIPEA (118.94 mg, 920.25 μmol, 160.29 μL, 3 eq). After stirring at 20° C. for 30 mins, methyl 6-[3-[2-[2-[2-[2-[2-(2-aminoethoxy)ethoxy]ethoxy]ethoxy]ethoxy]ethoxy]phenoxy]pyridine-3-carboxylate (156 mg, 306.75 μmol, 1 eq) was added. The resulting mixture was stirred at 20° C. for 1 h, then concentrated to the residue. H₂O (20 mL) was added, followed by extraction with ethyl acetate (20 mL×3). The combined organic phase was washed with brine (30 mL), dried with anhydrous Na₂SO₄, filtered and concentrated in vacuo. The residue was purified by silica gel chromatography eluted with (EtOAc:MeOH=50:1 to 10:1) to give methyl 6-[3-[2-[2-[2-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]ethoxy]ethoxy]ethoxy]phenoxy]pyridine-3-carboxylate (88.73% yield) as a yellow oil.

LCMS (ESI) [M+H]⁺ m/z: calc'd 735.3, found 735.3

¹H NMR (400 MHZ, DMSO-d₆) δ: ppm 8.71 (1H, d, J=2.4 Hz) 8.31 (1H, dd, J=8.6, 2.5 Hz) 7.83 (1H, br t, J=5.6 Hz) 7.35 (1H, t, J=8.1 Hz) 7.12 (1H, d, J=8.8 Hz) 6.86 (1H, dd, J=8.1, 2.1 Hz) 6.80 (1H, t, J=2.3 Hz) 6.76 (1H, dd, J=7.9, 1.8 Hz) 6.42 (1H, s) 6.36 (1H, s) 4.25-4.33 (1H, m) 4.07-4.15 (3H, m) 3.86 (3H, s) 3.70-3.77 (2H, m) 3.56-3.61 (2H, m) 3.47-3.55 (13H, m) 3.36-3.43 (2H, m) 3.14-3.21 (4H, m) 3.05-3.12 (1H, m) 2.82 (1H, dd, J=12.4, 5.1 Hz) 2.06 (2H, t, J=7.4 Hz) 1.56-1.67 (1H, m) 1.38-1.55 (3H, m) 1.15-1.36 (2H, m)

### Step 6:6-[3-[2-[2-[2-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]ethoxy]ethoxy]ethoxy]phenoxy]pyridine-3-carboxylic acid

To a solution of methyl 6-[3-[2-[2-[2-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]ethoxy]ethoxy]ethoxy]phenoxy]pyridine-3-carboxylate (150 mg, 204.12 μmol, 1 eq) in THF (1 mL), MeOH (0.5 mL) and H₂O (0.5 mL) was added LiOH·H₂O (25.70 mg, 612.37 μmol, 3 eq). The mixture was stirred at 25° C. for 2 hr. The reaction mixture was concentrated to the residue and acidified with 1N HCl until pH 1, then extracted with EtOAc (3×10 mL). The combined organic layers were washed with brine (15 mL), dried over anhydrous Na₂SO₄, filtered and concentrated in vacuo to give 6-[3-[2-[2-[2-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]ethoxy]ethoxy]ethoxy]phenoxy]pyridine-3-carboxylic acid (121 mg, 77.69% yield, 94.47% purity) as a yellow oil.

LCMS (ESI) [M+H]⁺ m/z: calc'd 721.3, found 721.2

¹H NMR (400 MHZ, METHANOL-d₄) δ: ppm 8.64 (1H, d, J=1.9 Hz) 8.25 (1H, dd, J=8.8, 2.4 Hz) 7.25 (1H, t, J=8.2 Hz) 6.91 (1H, d, J=8.6 Hz) 6.77 (1H, dd, J=8.3, 1.7 Hz) 6.57-6.72 (2H, m) 4.34-4.43 (1H, m) 4.19 (1H, dd, J=7.9, 4.4 Hz) 4.01-4.08 (2H, m) 3.70-3.78 (2H, m) 3.58-3.66 (3H, m) 3.48-3.56 (13H, m) 3.37-3.44 (2H, m) 3.23-3.27 (3H, m) 3.00-3.14 (1H, m) 2.81 (1H, dd, J=12.8, 5.0 Hz) 2.60 (1H, d, J=12.6 Hz) 2.11 (2H, t, J=7.3 Hz) 1.73-1.80 (1H, m) 1.45-1.57 (3H, m) 1.31-1.38 (2H, m)

### Step 7: (2,5-dioxopyrrolidin-1-yl) 6-[3-[2-[2-[2-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]ethoxy]ethoxy]ethoxy]phenoxy]pyridine-3-carboxylate

To a solution of 6-[3-[2-[2-[2-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]ethoxy]ethoxy]ethoxy]phenoxy]pyridine-3-carboxylic acid (120 mg, 166.48 μmol, 1 eq) and 1-hydroxypyrrolidine-2,5-dione (47.90 mg, 416.19 μmol, 2.5 eq) in DMF (5 mL) was added EDCI (47.87 mg, 249.71 μmol, 1.5 eq). The reaction mixture was stirred at 20° C. under N₂ for 3 h. LCMS showed the reaction was completed. The reaction mixture was poured into water (10 mL) and extracted with EtOAc (10 mL×3). The combined organic layers were washed with brine (10 mL), dried over anhydrous Na₂SO₄, filtered and concentrated in vacuo to give crude (2,5-dioxopyrrolidin-1-yl) 6-[3-[2-[2-[2-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]ethoxy]ethoxy]ethoxy]phenoxy]pyridine-3-carboxylate (136.16 mg, 100.00% yield) as a yellow oil.

LCMS (ESI) [M+H]⁺ m/z: calc'd 818.3, found 818.3

### Step 8: 2-[[6-[3-[2-[2-[2-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]ethoxy]ethoxy]ethoxy]phenoxy]pyridine-3-carbonyl]amino]-5,5-dimethyl-hexanoic acid

To a solution of (2,5-dioxopyrrolidin-1-yl) 6-[3-[2-[2-[2-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]ethoxy]ethoxy]ethoxy]phenoxy]pyridine-3-carboxylate (192.89 mg, 235.84 μmol, 1 eq), (2S)-2-amino-5,5-dimethyl-hexanoic acid (37.93 mg, 238.19 μmol, 1.01 eq) and DIEA (60.96 mg, 471.67 μmol, 82.16 μL, 2 eq) in DMF (3 mL), DCM (0.5 mL) and H₂O (1.5 mL). The reaction was degassed and purged with N₂ three times, and then stirred at 25° C. for 5 hrs under an N₂ atmosphere. The reaction mixture was concentrated and purified by prep_HPLC (column: Boston Green ODS 150*30 mm*5 um; mobile phase: [water (0.05% HCl)-ACN]; B %: 22%-62%, 9 min). After purification, the eluent was concentrated to remove organic solvents and the residual aqueous solution was lyophilized to give 2-[[6-[3-[2-[2-[2-[2-[2-[2-[5-[(3aS,4S,6aR)-2-oxo-1,3,3a,4,6,6a-hexahydrothieno[3,4-d]imidazol-4-yl]pentanoylamino]ethoxy]ethoxy]ethoxy]ethoxy]ethoxy]ethoxy]phenoxy]pyridine-3-carbonyl]amino]-5,5-dimethyl-hexanoic acid (20 mg, 9.76% yield, 99.2% purity) as a white solid.

LCMS (ESI) [M+H]⁺ m/z: calc'd 862.4, found 862.5

¹H NMR (400 MHZ, CHLOROFORM-d) 8 ppm 8.80 (1H, br s) 8.32 (1H, br s) 8.08 (1H, br s) 7.31 (2H, br t, J=7.8 Hz) 6.88-7.15 (2H, m) 6.67-6.86 (3H, m) 4.20-4.79 (6H, m) 4.11 (2H, br s) 3.84 (2H, br s) 3.71 (2H, br s) 3.63 (13H, br s) 3.50 (2H, br s) 3.38 (2H, br s) 3.11 (1H, br s) 2.65-2.96 (2H, m) 2.20 (2H, br s) 1.77-1.99 (2H, m) 1.62 (4H, br d, J=7.3 Hz) 1.34 (4H, br d, J=5.8 Hz) 0.88 (9H, s)

¹H NMR (400 MHZ, DMSO-d₆) δ ppm 8.56-8.75 (2H, m) 8.28 (1H, dd, J=8.7, 2.4 Hz) 7.81 (1H, br t, J=5.6 Hz) 7.33 (1H, t, J=8.2 Hz) 7.08 (1H, d, J=8.7 Hz) 6.84 (1H, dd, J=8.1, 2.0 Hz) 6.64-6.78 (2H, m) 6.40 (1H, br s) 4.26-4.34 (2H, m) 4.05-4.15 (3H, m) 3.71-3.75 (4H, m) 3.66 (9H, br s) 3.58 (4H, br dd, J=5.8, 3.1 Hz) 3.52-3.55 (3H, m) 3.38 (2H, br t, J=5.9 Hz) 3.17 (3H, q, J=5.8 Hz) 3.04-3.11 (1H, m) 2.81 (1H, dd, J=12.4, 5.1 Hz) 2.57 (3H, br d, J=12.3 Hz) 1.55-1.86 (4H, m) 1.37-1.54 (4H, m) 1.15-1.31 (4H, m) 0.87 (8H, s)

### BF010

### Synthesis of (2S)-2-[[6-[[5-[3-[3-[2-[2-[2-[2-(2,4-dinitroanilino)ethoxy]ethoxy]ethoxy]ethoxy]propanoylamino]propylcarbamoyl]-1-naphthyl]oxy]pyridine-3-carbonyl]amino]-5,5-dimethyl-hexanoic acid

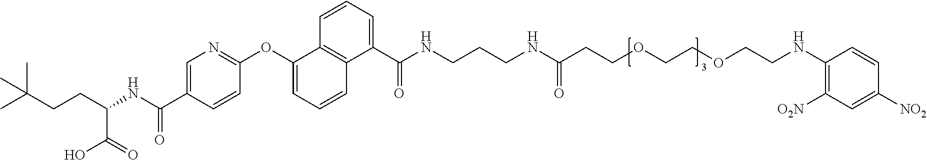

### Step 1: General Procedure for Preparation of (2,5-dioxopyrrolidin-1-yl) 5-bromonaphthalene-1-carboxylate

To a solution of 5-bromonaphthalene-1-carboxylic acid (2 g, 7.97 mmol, 1 eq) in DMF (20 mL) was added 1-hydroxypyrrolidine-2,5-dione (2.29 g, 19.91 mmol, 2.5 eq) and EDCI (2.29 g, 11.95 mmol, 1.5 eq), then the mixture was stirred at 15° C. for 2 hr. The reaction was poured into H₂O (40 mL) and filtered, the filter cake was washed with H₂O (60 mL) and dried in vacuum to give (2, 5-dioxopyrrolidin-1-yl) 5-bromonaphthalene-1-carboxylate (2.4 g, 6.89 mmol, 86.54% yield) as a white solid.

¹H NMR (400 MHZ, DMSO-d6) δ ppm 2.95 (s, 4H), 7.68 (dd, J=8.63, 7.63 Hz, 1H), 7.91 (dd, J=8.63, 7.38 Hz, 1H), 8.09 (dd, J=7.50, 0.75 Hz, 1H), 8.43 (dd, J=7.25, 1.00 Hz, 1H), 8.62 (dd, J=8.63, 5.50 Hz, 2H).

### Step 2: General Procedure for Preparation of tert-butyl N-[3-[(5-bromonaphthalene-1-carbonyl)amino]propyl]carbamate

To a solution of (2, 5-dioxopyrrolidin-1-yl) 5-bromonaphthalene-1-carboxylate (2.2 g, 6.32 mmol, 1 eq) in DMF (33 mL) was added tert-butyl N-(3-aminopropyl) carbamate (1.10 g, 6.32 mmol, 1.10 mL, 1 eq) and DIEA (2.45 g, 18.96 mmol, 3.30 mL, 3 eq). The mixture was stirred at 15° C. for 12 hr. The reaction was poured into H₂O (40 mL) and filtered, the filter cake was washed with H₂O (60 mL) and dried in vacuum to tert-butyl N-[3-[(5-bromonaphthalene-1-carbonyl)amino]propyl]carbamate (2.7 g, crude) as a white solid.

LCMS (ESI⁺): m/z 351.1 (M+H)

### Step 3: General Procedure for Preparation of tert-butyl N-[3-[(5-hydroxynaphthalene-1-carbonyl)amino]propyl]carbamate

A mixture of tert-butyl N-[3-[(5-bromonaphthalene-1-carbonyl)amino]propyl]carbamate (2.5 g, 6.14 mmol, 1 eq) and KOH (1.38 g, 24.55 mmol, 4 eq) in dioxane (25 mL) and H₂O (25 mL) was added tBuXPhos Pd G3 (243.79 mg, 306.90 μmol, 0.05 eq) was degassed and purged with N₂ for 3 times, and then the mixture was stirred at 110° C. for 12 hr under N₂ atmosphere. The insoluble was removed by filtration through celite, which was then washed with EA (100 ml). The mixture was diluted with water 100 mL, and then extracted with EtOAc 300 mL (100 mL*3). The combined organic layers were washed with brine 300 mL, dried over Na₂SO₄, filtered and concentrated under reduced pressure to give a residue. The mixture was purified by column chromatography (SiO₂, Petroleum ether/Ethyl acetate=3/1 to 1/2) to give tert-butyl N-[3-[(5-hydroxynaphthalene-1-carbonyl)amino]propyl]carbamate (2.1 g, 6.10 mmol, 99.34% yield) as a yellow solid.

### Data:

LCMS (ESI⁺): m/z 289.2 (M-55)

### Step 4: General Procedure for Preparation of methyl 6-[[5-[3-(tert-butoxycarbonylamino) propylcarbamoyl]-1-naphthyl]oxy]pyridine-3-carboxylate

A mixture of tert-butyl N-[3-[(5-hydroxynaphthalene-1-carbonyl)amino]propyl]carbamate (2.1 g, 6.10 mmol, 1 eq), methyl 6-fluoropyridine-3-carboxylate (1.14 g, 7.32 mmol, 1.2 eq) and Cs₂CO₃ (3.97 g, 12.19 mmol, 2 eq) in DMF (30 mL) was stirred at 80° C. for 12 hr. The mixture was diluted with water 30 mL, and then extracted with EtOAc 90 mL (30 mL*3). The combined organic layers were washed with brine 50 mL, dried over Na₂SO₄, filtered and concentrated under reduced pressure to give a residue. The residue was purified by column chromatography (SiO₂, Petroleum ether/Ethyl acetate=3/1 to 1/2) to afford methyl 6-[[5-[3-(tert-butoxycarbonylamino) propylcarbamoyl]-1-naphthyl]oxy]pyridine-3-carboxylate (1.1 g, 2.29 mmol, 37.62% yield) as a light yellow solid.

LCMS (ESI⁺): m/z 480.3 (M+H)

### Step 5: General Procedure for Preparation of methyl 6-[[5-(3-aminopropylcarbamoyl)-1-naphthyl]oxy]pyridine-3-carboxylate

Methyl 6-[[5-[3-(tert-butoxycarbonylamino) propyl carbamoyl]-1-naphthyl]oxy]pyridine-3-carboxylate (300 mg, 625.62 μmol, 1 eq) was dissolved in TFA (0.2 mL) and DCM (1 mL), and the reaction was stirred at 20° C. for 1 hr. The mixture was concentrated under reduced pressure to give methyl 6-[[5-(3-aminopropylcarbamoyl)-1-naphthyl]oxy]pyridine-3-carboxylate (300 mg, 607.99 μmol, 97.18% yield, TFA) as a colorless oil, which was used to next step without purification.

LCMS (ESI⁺): m/z 379.15 (M+H)

### Step 6: General Procedure for Preparation of methyl 6-[[5-[3-[3-[2-[2-[2-[2-(tert-butoxycarbonylamino)ethoxy]ethoxy]ethoxy]ethoxy]propanoylamino]propylcarbamoyl]-1-naphthyl]oxy]pyridine-3-carboxylate

To a combined solution of methyl 6-[[5-(3-aminopropylcarbamoyl)-1-naphthyl]oxy]pyridine-3-carboxylate (300 mg, 607.99 μmol, 1 eq, TFA), 3-[2-[2-[2-[2-(tert-butoxycarbonylamino)ethoxy]ethoxy]ethoxy]ethoxy]propanoic acid (222.17 mg, 607.99 μmol, 1 eq) and DIEA (235.73 mg, 1.82 mmol, 317.70 uL, 3 eq) in DMF (5 mL) was added HATU (346.76 mg, 911.98 μmol, 1.5 eq) at 0° C., then the reaction was stirred at 20° C. for 12 hr. Water (5 mL) was added, and the mixture was extracted with EtOAc (3*5 mL). The organic phase was washed with brine 30 mL (10 mL*3), dried over Na₂SO₄, filtered, concentrated and purified by prep-TLC (SiO₂, PE:EA=0:1) to give methyl 6-[[5-[3-[3-[2-[2-[2-[2-(tert-butoxycarbonylamino)ethoxy]ethoxy]ethoxy]ethoxy]propanoylamino]propylcarbamoyl]-1-naphthyl]oxy]pyridine-3-carboxylate (350 mg, 481.56 μmol, 79.20% yield) as a light yellow solid.

LCMS (ESI⁺): m/z 726.35 (M+H)

### Step 7: General Procedure for Preparation of methyl 6-[[5-[3-[3-[2-[2-[2-(2-aminoethoxy)ethoxy]ethoxy]ethoxy]propanoylamino]propylcarbamoyl]-1-naphthyl]oxy]pyridine-3-carboxylate

A solution of methyl 6-[[5-[3-[3-[2-[2-[2-[2-(tert-butoxycarbonylamino)ethoxy]ethoxy]ethoxy]ethoxy]propanoylamino]propylcarbamoyl]-1-naphthyl]oxy]pyridine-3-carboxylate (350 mg, 481.56 μmol, 1 eq) in TFA (0.2 mL) and DCM (2 mL) was stirred at 20° C. for 1 hr. The mixture was concentrated under reduced pressure to give methyl 6-[[5-[3-[3-[2-[2-[2-(2-aminoethoxy)ethoxy]ethoxy]ethoxy]propanoylamino]propylcarbamoyl]-1-naphthyl]oxy]pyridine-3-carboxylate (160 mg, 216.01 μmol, 44.86% yield, TFA) as a white solid, which was used to next step without purification.

LCMS (ESI⁺): m/z 626.3 (M+H)

### Step 8: General Procedure for Preparation of methyl 6-[[5-[3-[3-[2-[2-[2-[2-(2,4-dinitroanilino)ethoxy]ethoxy]ethoxy]ethoxy]propanoylamino]propylcarbamoyl]-1-naphthyl]oxy]pyridine-3-carboxylate

A mixture of methyl 6-[[5-[3-[3-[2-[2-[2-(2-aminoethoxy)ethoxy]ethoxy]ethoxy]propanoylamino]propylcarbamoyl]-1-naphthyl]oxy]pyridine-3-carboxylate (160 mg, 255.31 μmol, 1 eq), 1-fluoro-2,4-dinitro-benzene (71.27 mg, 382.96 μmol, 48.15 uL, 1.5 eq) and K₂CO₃ (105.86 mg, 765.92 μmol, 3 eq) in DMF (1 mL) was stirred at 20° C. for 2 hr. To the mixture was added of water (3 mL) and then extraction with ethyl acetate (3 mL). The organic phase was washed with brine 9 mL (3 mL*3), dried over Na₂SO₄, filtered and concentrated under reduced pressure to give a residue. The residue was purified by prep-HPLC (HCl condition; column: Phenomenex luna C18 80*40 mm*3 um; mobile phase: [water (HCl)-ACN]; B %: 40%-70%,7 min) to give methyl 6-[[5-[3-[3-[2-[2-[2-[2-(2,4-dinitroanilino)ethoxy]ethoxy]ethoxy]ethoxy]propanoylamino]propylcarbamoyl]-1-naphthyl]oxy]pyridine-3-carboxylate (130 mg, 156.77 μmol, 61.40% yield, HCl) as a yellow solid.

LCMS (ESI⁺): m/z 792.3 (M+H)

### Step 9: General Procedure for Preparation of 6-[[5-[3-[3-[2-[2-[2-[2-(2,4-dinitroanilino)ethoxy]ethoxy]ethoxy]ethoxy]propanoylamino]propylcarbamoyl]-1-naphthyl]oxy]pyridine-3-carboxylic acid

A solution of methyl 6-[[5-[3-[3-[2-[2-[2-[2-(2,4-dinitroanilino)ethoxy]ethoxy]ethoxy]ethoxy]propanoylamino]propylcarbamoyl]-1-naphthyl]oxy]pyridine-3-carboxylate (130 mg, 156.77 μmol, 1 eq, HCl) and LiOH·H₂O (13.16 mg, 313.54 μmol, 2 eq) in THF (1.5 mL) and H₂O (0.5 mL) was stirred at 20° C. for 12 hr. The resulting mixture was adjusted to pH=2 with 1M HCl, and extracted with EA 6 mL (2 mL*3), the organic phase was dried over Na₂SO₄, filtered and concentrated to give 6-[[5-[3-[3-[2-[2-[2-[2-(2,4-dinitroanilino)ethoxy]ethoxy]ethoxy]ethoxy]propanoylamino]propylcarbamoyl]-1-naphthyl]oxy]pyridine-3-carboxylic acid (120 mg, 147.20 μmol, 93.90% yield, HCl) as a yellow solid.

LCMS (ESI⁺): m/z 778.28 (M+H)

### Step 10: General Procedure for Preparation of (2,5-dioxopyrrolidin-1-yl) 6-[[5-[3-[3-[2-[2-[2-[2-(2,4-dinitroanilino)ethoxy]ethoxy]ethoxy]ethoxy]propanoylamino]propylcarbamoyl]-1-naphthyl]oxy]pyridine-3-carboxylate

To a solution of 1-hydroxypyrrolidine-2,5-dione (33.88 mg, 294.40 μmol, 2 eq) and 6-[[5-[3-[3-[2-[2-[2-[2-(2,4-dinitroanilino)ethoxy]ethoxy]ethoxy]ethoxy]propanoylamino]propylcarbamoyl]-1-naphthyl]oxy]pyridine-3-carboxylic acid (120 mg, 147.20 μmol, 1 eq, HCl) in DMF (1 mL) was added EDCI (42.33 mg, 220.80 μmol, 1.5 eq). The mixture was stirred at 20° C. for 1 hr. The reaction mixture was diluted with H₂O 3 mL and extracted with EtOAc 9 mL (3 mL*3). The combined organic layers were washed with brine (9 mL*3), dried over anhydrous Na₂SO₄, filtered and concentrated to give (2,5-dioxopyrrolidin-1-yl) 6-[[5-[3-[3-[2-[2-[2-[2-(2,4-dinitroanilino)ethoxy]ethoxy]ethoxy]ethoxy]propanoylamino]propylcarbamoyl]-1-naphthyl]oxy]pyridine-3-carboxylate (100 mg, 114.18 μmol, 77.57% yield) as a yellow oil, which was used to next step without purification.

LCMS (ESI⁺): m/z 875.3 (M+H)

### Step 11: General Procedure for Preparation of (2S)-2-[[6-[[5-[3-[3-[2-[2-[2-[2-(2,4-dinitroanilino)ethoxy]ethoxy]ethoxy]ethoxy]propanoylamino]propylcarbamoyl]-1-naphthyl]oxy]pyridine-3-carbonyl]amino]-5,5-dimethyl-hexanoic acid

To a solution of (2,5-dioxopyrrolidin-1-yl) 6-[[5-[3-[3-[2-[2-[2-[2-(2,4-dinitroanilino)ethoxy]ethoxy]ethoxy]ethoxy]propanoylamino]propylcarbamoyl]-1-naphthyl]oxy]pyridine-3-carboxylate (100 mg, 114.18 μmol, 1 eq) and (2S)-2-amino-5,5-dimethyl-hexanoic acid (18.18 mg, 114.18 μmol, 1 eq) in DMF (0.6 mL), DCM (0.1 mL) and H₂O (0.3 mL) was added DIEA (22.13 mg, 171.27 μmol, 29.83 uL, 1.5 eq), the reaction was degassed and purged with N₂ for 3 times, and stirred at 20° C. for 12 hr under N₂ atmosphere. The reaction was concentrated under reduced pressure and the resulting mixture was adjusted to pH=3, which was purified by prep-HPLC (HCl condition; column: Phenomenex Luna 80*30 mm*3 um; mobile phase: [water (HCl)-ACN]; B %: 25%-55%, 8 min) to give (2S)-2-[[6-[5-[3-[3-[2-[2-[2-[2-(2,4-dinitroanilino)ethoxy]ethoxy]ethoxy]ethoxy]propanoylamino]propylcarbamoyl]-1-naphthyl]oxy]pyridine-3-carbonyl]amino]-5,5-dimethyl-hexanoic acid (78.4 mg, 81.97 umol, 71.79% yield, 100% purity, HCl) as a yellow solid.

LCMS (ESI⁺): m/z 919.4 (M+H)

¹H NMR (400 MHZ, METHANOL-d4) δ=8.99 (d, J=2.8 Hz, 1H), 8.63-8.57 (m, 1H), 8.36-8.30 (m, 1H), 8.23 (dd, J=2.7, 9.6 Hz, 1H), 8.16 (d, J=8.5 Hz, 1H), 8.00 (d, J=8.5 Hz, 1H), 7.69 (dd, J=1.0, 7.0 Hz, 1H), 7.65-7.59 (m, 1H), 7.52 (dd, J=7.1, 8.4 Hz, 1H), 7.36 (dd, J=0.8, 7.5 Hz, 1H), 7.18-7.11 (m, 2H), 4.56-4.47 (m, 1H), 3.78-3.71 (m, 4H), 3.66-3.58 (m, 14H), 3.53 (t, J=6.8 Hz, 2H), 3.37 (t, J=6.8 Hz, 2H), 2.48 (t, J=6.1 Hz, 2H), 2.01-1.77 (m, 4H), 1.39-1.29 (m, 2H), 0.94-0.90 (m, 9H).

### BF031

### 1. General Procedure for Preparation of methyl 6-(3-tert-butoxycarbonylphenoxy)pyridine-3-carboxylate

To a solution of tert-butyl 3-hydroxybenzoate (2.8 g, 14.42 mmol, 1 eq), methyl 6-fluoropyridine-3-carboxylate (2.24 g, 14.42 mmol, 1 eq) in DMF (30 mL) was added Cs₂CO₃ (7.05 g, 21.62 mmol, 1.5 eq). The mixture was stirred at 80° C. for 2 hr. The reaction mixture was quenched by addition H₂O 30 mL, and extracted with EA 90 mL (30 mL*3). The combined organic layers were washed with brine 100 mL (20 mL*5), dried over Na₂SO₄, filtered and concentrated under reduced pressure. The residue was purified by column chromatography (SiO₂, Petroleum ether/Ethyl acetate=1/0 to 0/1) to give methyl 6-(3-tert-butoxycarbonylphenoxy) pyridine-3-carboxylate (4.5 g, 13.66 mmol, 94.78% yield) as a colorless oil.

### Data:

LCMS (ESI⁺): m/z 330.1 (M+H)

### 2. General Procedure for Preparation of 6-(3-tert-butoxycarbonylphenoxy)pyridine-3-carboxylic acid

3. To a stirred solution of methyl 6-(3-(tert-butoxycarbonyl)phenoxy) nicotinate (4.42 g,
THF:H₂O 0° C.-rt 5 h
   13.42 mmol) in THF (45.0 mL) was added lithium hydroxide (0.64 g, 26.72 mmol) in water (25.0 mL) dropwise at 0° C. Then reaction mixture was slowly warmed to room temperature and stirred for 5 h. After completion of reaction (via TLC), the pH of the reaction mass was adjusted to 5-6 using 1.0 M HCl solution at 0° C. and extracted with ethyl acetate. The organic layer was dried over anhydrous Na₂SO₄, filtered and concentrated to afford crude compound. The crude was purified by column chromatography (eluted with 100% ethyl acetate) to afford 6-(3-(tert-butoxycarbonyl)phenoxy) nicotinic acid (Int-2) (3.80 g, 89%) as a white solid.

LCMS (ESI⁺): m/z 316.1 (M+H)

### 4. General Procedure for Preparation of tert-butyl 3-[[5-[[(1S)-1-methoxycarbonyl-4,4-dimethyl-pentyl]carbamoyl]-2-pyridyl]oxy]benzoate

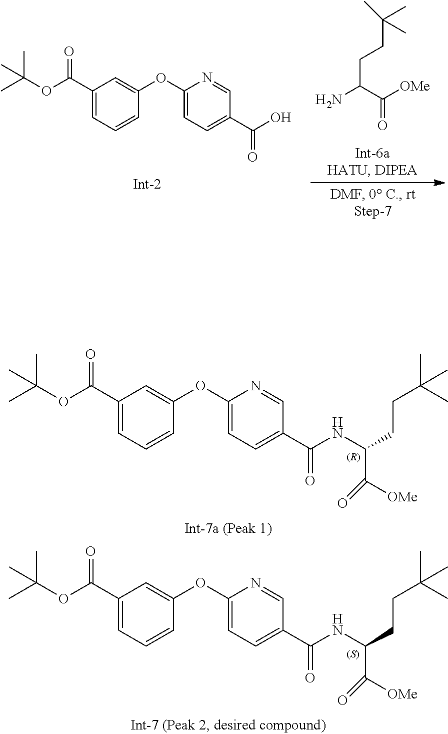

HATU (4.52 g, 11.89 mmol) was added to a solution of 6-(3-tert-butoxycarbonylphenoxy)pyridine-3-carboxylic acid (3.80 g, 12.05 mmol), methyl 2-amino-5,5-dimethylhexanoate (Int-6a) (2.51 g, 14.49 mmol) and DIPEA (3.49 mL, 36.36 mmol) in N,N-dimethylformamide (38.0 mL) at 0° C. The reaction mixture was stirred at room temperature for 3 h. After completion of reaction (via TLC), the reaction mass was quenched with ice water and extracted with ethyl acetate. The organic layer was dried over anhydrous Na₂SO₄, filtered and concentrated to afford crude compound. The crude was purified by column chromatography (eluted with 10-50% ethyl acetate in pet ether) to afford tert-butyl (rac)-3-((5-((1-methoxy-5, 5-dimethyl-1-oxohexan-2-yl)carbamoyl)pyridin-2-yl)oxy)benzoate (3.0 g). Isomers are separated by chiral SFC to afford tert-butyl(S)-3-((5-((1-methoxy-5,5-dimethyl-1-oxohexan-2-yl)carbamoyl)pyridin-2-yl)oxy)benzoate (desired compound) (1.3 g, 40%) as a white solid and tert-butyl (R)-3-((5-((1-methoxy-5,5-dimethyl-1-oxohexan-2-yl)carbamoyl)pyridin-2-yl)oxy)benzoate (1.2 g, 38%) as a white solid.

LCMS (m/z): 371.58 [M+H]+

### 5. General Procedure for Preparation of 3-[[5-[[(1S)-1-methoxycarbonyl-4,4-dimethyl-pentyl]carbamoyl]-2-pyridyl]oxy]benzoic acid

4.0M HCl in 1,4-dioxane (14.0 mL) was added to a stirred solution of tert-butyl(S)-3-((5-((1-methoxy-5,5-dimethyl-1-oxohexan-2-yl)carbamoyl)pyridin-2-yl)oxy)benzoate (1.40 g, 2.97 mmol) in dichloromethane (28.0 mL) at 0° C. The reaction mixture was slowly warmed to room temperature and stirred for 24 h. After completion of reaction (via TLC), the reaction mass was quenched with ice water, the pH of the aqueous was adjusted to 9-10 using saturated solution of NaHCO3 and extracted with dichloromethane. The organic layer was dried over anhydrous Na2SO4, filtered and concentrated to afford crude compound. The crude was purified by column chromatography (eluted with 100% ethyl acetate) to afford(S)-3-((5-((1-methoxy-5,5-dimethyl-1-oxohexan-2-yl)carbamoyl)pyridin-2-yl)oxy)benzoic acid (1.01 g, 82%) as white solid.

1H NMR (400 MHZ, DMSO) § 13.16 (s, 1H), 8.82 (d, J=7.5 Hz, 1H), 8.62 (d, J=2.1 Hz, 1H), 8.31 (dd, J=2.4, 8.4 Hz, 1H), 7.83 (d, J=7.7 Hz, 1H), 7.65 (t, J=1.8 Hz, 1H), 7.58 (t, J=7.9 Hz, 1H), 7.46 (dd, J=2.4, 7.2 Hz, 1H), 7.20 (d, J=8.6 Hz, 1H), 4.37 (q, J=7.3 Hz, 1H), 3.65 (s, 3H), 1.82-1.67 (m, 2H), 1.35-1.15 (m, 2H), 0.87 (s, 9H). LCMS (m/z): 415.36 [M+H]+

### 6. General Procedure for Preparation of tert-butyl N-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxoethoxy]ethoxy]ethoxy]ethoxy]ethyl]carbamate

To a solution of 3-[2,2-dimethyl-6-[2-[(3R)-3-methylpiperazin-1-yl]pyrimidin-5-yl]-3-oxo-pyrrolo[2,3-b]pyridin-1-yl]methyl]pyridine-2-carbonitrile (305 mg, 536.45 μmol, 1 eq, TFA), 2-[2-[2-[2-[2-(tert-butoxycarbonylamino)ethoxy]ethoxy]ethoxy]ethoxy]acetic acid (207.36 mg, 590.10 μmol, 1.1 eq) and DIEA (207.99 mg, 1.61 mmol, 280.31 uL, 3 eq) in DMF (3 mL) was added HATU (305.96 mg, 804.68 μmol, 1.5 eq) at 0° C., then the reaction was stirred for 1 hr at 20° C. The reaction mixture was partitioned between water 10 mL and EtOAc 10 mL. The organic phase washed with brine 30 mL (10 mL*3), dried over Na₂SO₄, filtered and concentrated under reduced pressure to give tert-butyl N-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethyl]carbamate (400 mg, 507.68 μmol, 94.64% yield) as a yellow oil.

LCMS (ESI⁺): m/z 788.4

### 7. General Procedure for Preparation of 3-[[6-[2-[(3R)-4-[2-[2-[2-[2-(2-aminoethoxy)ethoxy]ethoxy]ethoxy]acetyl]-3-methyl-piperazin-1-yl]pyrimidin-5-yl]-2,2-dimethyl-3-oxo-pyrrolo[2,3-b]pyridin-1-yl]methyl]pyridine-2-carbonitrile

A solution of tert-butyl N-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethyl]carbamate (0.4 g, 507.68 μmol, 1 eq) in TFA (1 mL) and DCM (3 mL) was stirred at 15° C. for 12 hr. The reaction was concentrated to give 3-[[6-[2-[(3R)-4-[2-[2-[2-[2-(2-aminoethoxy)ethoxy]ethoxy]ethoxy]acetyl]-3-methyl-piperazin-1-yl]pyrimidin-5-yl]-2,2-dimethyl-3-oxo-pyrrolo[2,3-b]pyridin-1-yl]methyl]pyridine-2-carbonitrile (400 mg, 498.87 μmol, 98.27% yield, TFA) as a yellow oil.

LCMS (ESI⁺): m/z 688.5

### 8. General Procedure for Preparation of (2S)-2-[[6-[3-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethylcarbamoyl]phenoxy]pyridine-3-carbonyl]amino]-5,5-dimethyl-hexanoate

To a solution of 3-[[6-[2-[(3R)-4-[2-[2-[2-[2-(2-aminoethoxy)ethoxy]ethoxy]ethoxy]acetyl]-3-methyl-piperazin-1-yl]pyrimidin-5-yl]-2,2-dimethyl-3-oxo-pyrrolo[2,3-b]pyridin-1-yl]methyl]pyridine-2-carbonitrile (200 mg, 249.44 umol, 1 eq, TFA), 3-[[5-[[(1S)-1-methoxycarbonyl-4,4-dimethyl-pentyl]carbamoyl]-2-pyridyl]oxy]benzoic acid (124.05 mg, 234.74 μmol, 9.41e-1 eq, TFA) and DIEA (96.71 mg, 748.31 μmol, 130.34 uL, 3 eq) in DMF (3 mL) was added HATU (142.26 mg, 374.15 μmol, 1.5 eq) at 0° C., then the reaction was stirred for 2 hr at 15° C. The reaction mixture was diluted with H₂O 3 mL, and extracted with EA 30 mL (3 mL*10). The combined organic layers washed with brine 15 mL (5 mL*3), dried over Na₂SO₄, filtered and concentrated under reduced pressure to give methyl (2S)-2-[[6-[3-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethylcarbamoyl]phenoxy]pyridine-3-carbonyl]amino]-5,5-dimethyl-hexanoate (300 mg, crude) as a yellow oil.

LCMS (ESI⁺): m/z 1084.6 (M+H)

### 9. General Procedure for Preparation of (2S)-2-[[6-[3-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethylcarbamoyl]phenoxy]pyridine-3-carbonyl]amino]-5,5-dimethyl-hexanoic acid

To a solution of methyl (2S)-2-[[6-[3-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethylcarbamoyl]phenoxy]pyridine-3-carbonyl]amino]-5,5-dimethyl-hexanoate (300 mg, 276.70 μmol, 1 eq) in THF (2 mL) and H₂O (1 mL) was added LiOH·H₂O (23.22 mg, 553.39 μmol, 2 eq). The mixture was stirred at 20° C. for 2 hr. The residue acid with HCl (1M) to pH=2 and purified by prep-HPLC (HCl condition: column: Phenomenex Luna 80*30 mm*3 um; mobile phase: [water (HCl)-ACN]; B %: 35%-65%, 8 min) to give (2S)-2-[[6-[3-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethylcarbamoyl]phenoxy]pyridine-3-carbonyl]amino]-5,5-dimethyl-hexanoic acid (57 mg, 49.44 μmol, 17.87% yield, 95.98% purity, HCl) as a yellow solid.

LCMS (ESI⁺): m/z 535.9 (M/2+1)

1H NMR (400 MHZ, METHANOL-d4) δ=8.95 (s, 2H), 8.63-8.59 (m, 2H), 8.26 (dd, J=2.4, 8.7 Hz, 1H), 8.02 (dd, J=1.3, 8.1 Hz, 1H), 7.95 (d, J=8.0 Hz, 1H), 7.71 (d, J=7.6 Hz, 1H), 7.63-7.61 (m, 1H), 7.61-7.58 (m, 1H), 7.51 (t, J=7.9 Hz, 1H), 7.34-7.28 (m, 2H), 7.06 (d, J=8.6 Hz, 1H), 5.11 (s, 2H), 4.74-4.55 (m, 2H), 4.51 (dd, J=5.1, 8.9 Hz, 1H), 4.40-4.13 (m, 3H), 3.91-3.82 (m, 1H), 3.67-3.58 (m, 14H), 3.57-3.53 (m, 2H), 3.47-3.36 (m, 2H), 3.12-2.98 (m, 1H), 2.02-1.90 (m, 1H), 1.88-1.75 (m, 1H), 1.38-1.31 (m, 8H), 1.25-1.13 (m, 3H), 0.91 (s, 9H)

The following compounds were prepared according to the general procedure. The starting materials are either commercially available or may be prepared from commercially available reagents using conventional reactions well known in the art.

| | | | LC/MS |
|---|---|---|---|
| ID. | Name | HNMR | [M + 1] |
| BF 051 | (2S)-2-[[6-[3-[2-[2-[2- | ¹H NMR (400 MHz, METHANOL-d4) | 982.5 |
| | [(2R)-4-[5-[1-[(2-cyano- | δ ppm 8.94 (s, 2 H), 8.57-8.65 (m, 2 | |
| | 3-pyridyl)methyl]-2,2- | H), 8.23 (dd, J = 8.63, 2.13 Hz, 1 H), | |
| | dimethyl-3-oxo-pyrrolo[2,3- | 8.00-8.06 (m, 1 H), 7.95 (d, J = 8.00 | |
| | b]pyridin-6-yl]pyrimidin- | Hz, 1 H), 7.72 (d, J = 7.75 Hz, 1 H), | |
| | 2-yl]-2-methyl-piperazin-1- | 7.58-7.65 (m, 2 H), 7.50 (br t, | |
| | yl]-2-oxo- | J = 7.69 Hz, 1 H), 7.30 (d, J = 8.00 Hz, | |
| | ethoxy]ethoxy]ethyl- | 2 H), 7.03 (br d, J = 8.50 Hz, 1 H), | |
| | carbamoyl]phenoxy]pyridine- | 5.12 (s, 2 H), 4.60-4.80 (m, 2 H), | |
| | 3-carbonyl]amino]-5,5- | 4.46-4.57 (m, 2 H), 4.16-4.34 (m, 2 | |
| | dimethyl-hexanoic acid | H), 3.63-3.74 (m, 6 H), 3.54-3.62 | |
| | | (m, 2 H), 3.36 (br d, J = 2.13 Hz, 2 H), | |
| | | 3.21-3.30 (m, 2 H), 1.89-2.01 (m, 1 | |
| | | H), 1.74-1.86 (m, 1 H), 1.30-1.41 | |
| | | (m, 8 H), 1.06-1.22 (m, 3 H), 0.91 | |
| | | (s, 9 H). | |
| BF056 | (2S)-2-[[6-[3-[2-[2-[2-[2- | 1H NMR (400 MHz, METHANOL-d4) | 1026.4 |
| | [(2R)-4-[5-[1-[(2-cyano- | δ ppm 8.94 (s, 2 H), 8.60 (td, J = 4.22, | |
| | 3-pyridyl)methyl]-2,2- | 1.81 Hz, 2 H), 8.25 (dd, J = 8.69, 2.44 | |
| | dimethyl-3-oxo-pyrrolo[2,3- | Hz, 1 H), 8.02 (d, J = 7.25 Hz, 1 H), | |
| | b]pyridin-6-yl]pyrimidin- | 7.94 (d, J = 8.00 Hz, 1 H), 7.70 (br d, | |
| | 2-yl]-2-methyl-piperazin-1- | J = 7.75 Hz, 1 H), 7.57-7.64 (m, 2 H), | |
| | yl]-2-oxo- | 7.50 (t, J = 7.94 Hz, 1 H), 7.26-7.34 | |
| | ethoxy]ethoxy]ethoxy]ethyl- | (m, 2 H), 7.05 (d, J = 8.63 Hz, 1 H), | |
| | carbamoyl]phenoxy] pyridine- | 5.11 (s, 2 H), 4.54-4.73 (m, 2 H), | |
| | 3-carbonyl]amino]-5,5- | 4.50 (dd, J = 8.82, 5.19 Hz, 1 H), 4.12- | |
| | dimethyl-hexanoic acid | 4.41 (m, 3 H), 3.81-3.91 (m, 0.5 | |
| | | H), 3.62-3.68 (m, 10 H), 3.54-3.58 | |
| | | (m, 2 H), 3.34-3.49 (m, 2 H), 3.21- | |
| | | 3.28 (m, 0.5 H), 2.96-3.10 (m, 1 H), | |
| | | 1.89-2.02 (m, 1 H), 1.74-1.87 (m, 1 | |
| | | H), 1.32-1.40 (m, 8 H), 1.11-1.23 | |
| | | (m, 3 H), 0.91 (s, 9 H). | |
| BF031 | (2S)-2-[[6-[3-[2-[2-[2-[2- | 1H NMR (400 MHz, METHANOL-d4) | 1070.6 |
| | [2-[(2R)-4-[5-[1-[(2-cyano- | δ = 8.95 (s, 2H), 8.63-8.59 (m, 2H), | |
| | 3-pyridyl)methyl]-2,2- | 8.26 (dd, J = 2.4, 8.7 Hz, 1H), 8.02 | |
| | dimethyl-3-oxo-pyrrolo[2,3- | (dd, J = 1.3, 8.1 Hz, 1H), 7.95 (d, J = | |
| | b]pyridin-6-yl]pyrimidin- | 8.0 Hz, 1H), 7.71 (d, J = 7.6 Hz, 1H), | |
| | 2-yl]-2-methyl-piperazin-1- | 7.63-7.61 (m, 1H), 7.61-7.58 (m, | |
| | yl]-2-oxo- | 1H), 7.51 (t, J = 7.9 Hz, 1H), 7.34- | |
| | ethoxy]ethoxy]ethoxy]ethoxy]ethyl- | 7.28 (m, 2H), 7.06 (d, J = 8.6 Hz, | |
| | carbamoyl]phenoxy]pyridine-3- | 1H), 5.11 (s, 2H), 4.74-4.55 (m, 2H), | |
| | carbonyl]amino]-5,5- | 4.51 (dd, J = 5.1, 8.9 Hz, 1H), 4.40- | |
| | dimethyl-hexanoic acid | 4.13 (m, 3H), 3.91-3.82 (m, 0.5H), | |
| | | 3.67-3.58 (m, 14H), 3.57-3.53 (m, | |
| | | 2H), 3.47-3.36 (m, 2.5H), 3.12- | |
| | | 2.98 (m, 1H), 2.02-1.90 (m, 1H), | |
| | | 1.88-1.75 (m, 1H), 1.38-1.31 (m, | |
| | | 8H), 1.25-1.13 (m, 3H), 0.91 (s, 9H). | |

### BF033

### 1. General Procedure for Preparation of methyl (2S)-2-amino-5,5-dimethyl-hexanoate

To a solution of (2S)-2-amino-5, 5-dimethyl-hexanoic acid (3 g, 18.84 mmol, 1 eq) in MeOH (40 mL) was added SOCl₂ (6.72 g, 56.52 mmol, 4.10 mL, 3 eq) dropwise at 0° C. After addition, the mixture was stirred at 15° C. for 16 hr. The reaction was concentrated to give methyl (2S)-2-amino-5, 5-dimethyl-hexanoate (3.26 g, crude) as a yellow solid.

### Data:

¹H NMR (400 MHZ, CHLOROFORM-d) δ ppm 0.91 (s, 9H), 1.27-1.51 (m, 2H), 2.03 (br d, J=3.75 Hz, 2H), 3.82 (s, 3H), 4.08 (br s, 1H).

### 2. General Procedure for Preparation of methyl 6-(3-tert-butoxycarbonylphenoxy)pyridine-3-carboxylate

To a solution of tert-butyl 3-hydroxybenzoate (2.8 g, 14.42 mmol, 1 eq), methyl 6-fluoropyridine-3-carboxylate (2.24 g, 14.42 mmol, 1 eq) in DMF (30 mL) was added Cs₂CO₃ (7.05 g, 21.62 mmol, 1.5 eq). The mixture was stirred at 80° C. for 2 hr. The reaction mixture was diluted with H₂O 30 mL, and extracted with EA 90 mL (30 mL*3). The combined organic layers were washed with brine 100 mL (20 mL*5), dried over Na₂SO₄, filtered and concentrated under reduced pressure. The residue was purified by column chromatography (SiO₂, Petroleum ether/Ethyl acetate=1/0 to 0/1) to afford methyl 6-(3-tert-butoxycarbonylphenoxy) pyridine-3-carboxylate (4.5 g, 13.66 mmol, 94.78% yield) as a colorless oil.

### Data:

LCMS (ESI⁺): m/z 330.1 (M+H)

### 3. 6-(3-tert-butoxycarbonylphenoxy)pyridine-3-carboxylic acid was Prepared as Described Above

### 4. tert-butyl 3-[[5-[[(1S)-1-methoxycarbonyl-4,4-dimethyl-pentyl]carbamoyl]-2-pyridyl]oxy]benzoate acid was Prepared as Described Above

### 5. General Procedure for Preparation of (2,5-dioxopyrrolidin-1-yl) 3-[[5-[[(1S)-1-methoxycarbonyl-4,4-dimethyl-pentyl]carbamoyl]-2-pyridyl]oxy]benzoate

To a solution of 3-[[5-[[(1S)-1-methoxycarbonyl-4,4-dimethyl-pentyl]carbamoyl]-2-pyridyl]oxy]benzoic acid (50 mg, 94.61 μmol, 1 eq, TFA) in DMF (1 mL) was added 1-hydroxypyrrolidine-2,5-dione (54.44 mg, 473.06 μmol, 5 eq) and EDCI (90.69 mg, 473.06 μmol, 5 eq). The mixture was stirred at 20° C. for 2 hr. The mixture was diluted with water 3 mL, and then extracted with Ethyl acetate 6 mL (2 mL*3). The combined organic layers were washed with brine 5 mL, dried over Na₂SO₄, filtered and concentrated under reduced pressure to give a residue which was purified by prep-TLC (SiO₂, Petroleum ether:Ethyl acetate=1:1) to afford (2,5-dioxopyrrolidin-1-yl) 3-[[5-[[(1S)-1-methoxycarbonyl-4,4-dimethyl-pentyl]carbamoyl]-2-pyridyl]oxy]benzoate (32 mg, 62.56 μmol, 66.12% yield) as a colorless oil.

### Data:

LCMS (ESI⁺): m/z 512.1 (M+H)

### 6. General Procedure for Preparation of 2-[2-[2-(tert-butoxycarbonylamino)ethoxy]ethoxy]ethyl 4-methylbenzenesulfonate

To a solution of tert-butyl N-[2-[2-(2-hydroxyethoxy)ethoxy]ethyl]carbamate (1 g, 4.01 mmol, 1 eq) in DCM (10 mL) was added TEA (811.78 mg, 8.02 mmol, 1.12 mL, 2 eq) and 4-methylbenzenesulfonyl chloride (1.53 g, 8.02 mmol, 2 eq) at 0° C. Then the mixture was stirred at 15° C. for 2 hr. The reaction mixture diluted with H₂O 10 mL, and extracted with Ethyl acetate 30 mL (10 mL*3). The combined organic layers dried over Na₂SO₄, filtered and concentrated under reduced pressure. The residue was purified by prep-TLC (SiO₂, Petroleum ether:Ethyl acetate=1:1) to give 2-[2-[2-(tert-butoxycarbonylamino)ethoxy]ethoxy]ethyl 4-methylbenzenesulfonate (1 g, 2.48 mmol, 61.79% yield) as a colorless oil.

### Data:

### 7. General Procedure for Preparation of tert-butyl N-[3-[(5-bromonaphthalene-1-carbonyl)amino]propyl]carbamate

To a solution of [4-(8-isoquinolyl)-3-[2-[4-(8-isoquinolyl)phenyl]ethoxy]phenyl]methanol (700 mg, 1.45 mmol, 1 eq) and 2-[2-[2-(tert-butoxycarbonylamino)ethoxy]ethoxy]ethyl 4-methylbenzenesulfonate (877.59 mg, 2.17 mmol, 1.5 eq) in DMF (13 mL) was added NaH (145.06 mg, 3.62 mmol, 60% purity, 2.5 eq) at 0° C. under N₂, then the reaction was stirred for 1 hr at 0° C. and 11 hr at 15° C. The mixture was quenched with saturated NH₄Cl solution (10 mL) at 0° C. and extracted with Ethyl acetate 30 mL (10 mL*3). The combined organic layers were washed with brine (20 mL*3), dried over anhydrous Na₂SO₄, filtered and concentrated under reduced pressure to give a residue. The residue was purified by prep-TLC (SiO₂, petroleum ether:ethyl acetate=0:1) to give tert-butyl N-[2-[2-[2-[[4-(8-isoquinolyl)-3-[2-[4-(8-isoquinolyl)phenyl]ethoxy]phenyl]methoxy]ethoxy]ethoxy]ethyl]carbamate as a yellow oil.

### Data:

LCMS (ESI⁺): m/z 714.2 (M+H)

### 10. General Procedure for Preparation of tert-butyl N-[3-[(5-hydroxynaphthalene-1-carbonyl)amino]propyl]carbamate

A solution of tert-butyl N-[2-[2-[2-[[4-(8-isoquinolyl)-3-[2-[4-(8-isoquinolyl)phenyl]ethoxy]phenyl]methoxy]ethoxy]ethoxy]ethyl]carbamate (550 mg, 770.46 μmol, 1 eq) in TFA (6 mL) was stirred for 1 hr at 15° C. The mixture was concentrated to get a crude product. 2-[2-[2-[4-(8-isoquinolyl)-3-[2-[4-(8-isoquinolyl)phenyl]ethoxy]phenyl]methoxy]ethoxy]ethoxylethanamine (560 mg, crude, TFA) was obtained as a yellow oil, which was used to next step without purification Data:

LCMS (ESI⁺): m/z 614.3 (M+H)

### 11. General Procedure for Preparation of methyl (2S)-2-[[6-[3-[2-[2-[2-[[4-(8-isoquinolyl)-3-[2-[4-(8-isoquinolyl)phenyl]ethoxy]phenyl]methoxy]ethoxy]ethoxy]ethylcarbamoyl]phenoxy]pyridine-3-carbonyl]amino]-5,5-dimethyl-hexanoate

To a solution of 2-[2-[2-[[4-(8-isoquinolyl)-3-[2-[4-(8-isoquinolyl)phenyl]ethoxy]phenyl]methoxy]ethoxy]ethoxy]ethanamine (39.84 mg, 54.74 umol, 1 eq, TFA) and (2,5-dioxopyrrolidin-1-yl) 3-[[5-[[(1S)-1-methoxycarbonyl-4,4-dimethyl-pentyl]carbamoyl]-2-pyridyl]oxy]benzoate (28 mg, 54.74 μmol, 1 eq) in DMSO (0.5 mL) was added DIEA (7.07 mg, 54.74 μmol, 9.53 uL, 1 eq) at 0° C. The mixture was stirred at 20° C. for 1 hr. The mixture was quenched by water 2 mL, and then extracted with DCM 3 mL (1 mL*3). The combined organic layers were washed with brine 2 mL, dried over Na₂SO₄, filtered and concentrated under reduced pressure to give a residue. The mixture was purified by prep-HPLC (HCl condition; column: Phenomenex Luna 80*30 mm*3 um; mobile phase: [water (HCl)-ACN]; B %: 30%-60%, 8 min) to give methyl (2S)-2-[[6-[3-[2-[2-[2-[[4-(8-isoquinolyl)-3-[2-[4-(8-isoquinolyl)phenyl]ethoxy]phenyl]methoxy]ethoxy]ethoxy]ethylcarbamoyl]phenoxy]pyridine-3-carbonyl]amino]-5,5-dimethyl-hexanoate (10 mg, 9.55 μmol, 17.45% yield, 100% purity, HCl) as a white solid.

### Data:

LCMS (ESI⁺): m/z 1010.2 (M+H)

### 12. General Procedure for Preparation of (2S)-2-[[6-[3-[2-[2-[2-[[4-(8-isoquinolyl)-3-[2-[4-(8-isoquinolyl)phenyl]ethoxy]phenyl]methoxy]ethoxy]ethoxy]ethylcarbamoyl]phenoxy]pyridine-3-carbonyl]amino]-5,5-dimethyl-hexanoic acid

To a solution of methyl (2S)-2-[[6-[3-[2-[2-[2-[[4-(8-isoquinolyl)-3-[2-[4-(8-isoquinolyl)phenyl]ethoxy]phenyl]methoxy]ethoxy]ethoxy]ethylcarbamoyl]phenoxy]pyridine-3-carbonyl]amino]-5,5-dimethyl-hexanoate (10 mg, 9.55 μmol, 1 eq, HCl) in THF (0.1 mL) and H₂O (0.1 mL) was added LiOH·H₂O (801.87 ug, 19.11 μmol, 2 eq). The mixture was stirred at 20° C. for 1 hr. The THF was removed under reduced pressure and the residue was acid with FA to pH=2. The mixture was purified by prep-HPLC (FA condition; column: Phenomenex Luna C18 200*40 mm*10 um; mobile phase: [water (FA)-ACN]; B %: 30%-70%, 8 min) to give (2S)-2-[[6-[3-[2-[2-[2-[[4-(8-isoquinolyl)-3-[2-[4-(8-isoquinolyl)phenyl]ethoxy]phenyl]methoxy]ethoxy]ethoxy]ethylcarbamoyl]phenoxy]pyridine-3-carbonyl]amino]-5,5-dimethyl-hexanoic acid (4.7 mg, 4.72 μmol, 49.38% yield, 100% purity) as a white solid.

### Data:

LCMS (ESI⁺): m/z 996.3 (M+H)

¹H NMR (400 MHZ, METHANOL-d4) δ ppm 9.02 (s, 1H), 8.85 (s, 1H), 8.56-8.60 (m, 1H), 8.43-8.48 (m, 1H), 8.36 (d, J=5.75 Hz, 1H), 8.22 (dd, J=8.63, 2.50 Hz, 1H), 7.94 (dd, J=8.25, 4.38 Hz, 2H), 7.89 (d, J=5.38 Hz, 1H), 7.79-7.86 (m, 3H), 7.71 (d, J=7.75 Hz, 1H), 7.63 (d, J=1.88 Hz, 1H), 7.48 (dt, J=11.35, 7.71 Hz, 3H), 7.24-7.29 (m, 2H), 7.17 (s, 1H), 7.05-7.10 (m, 3H), 7.00 (d, J=8.76 Hz, 1H), 6.79 (d, J=7.75 Hz, 2H), 4.62 (s, 2H), 4.50 (dd, J=8.63, 5.38 Hz, 1H), 4.22-4.29 (m, 1H), 4.11-4.18 (m, 1H), 3.64-3.72 (m, 10H), 3.54-3.57 (m, 2H), 2.71-2.84 (m, 2H), 1.90-2.01 (m, 1H), 1.72-1.86 (m, 1H), 1.29-1.36 (m, 2H), 0.89 (s, 9H).

The following compounds were prepared according to the general procedure.

The starting materials are either commercially available or may be prepared from commercially available reagents using conventional reactions well known in the art.

| Compound | | | LC/MS |
|---|---|---|---|
| No. | Name | HNMR | [M + 1] |
| BF033 | (2S)-2-[[6-[3-[2-[2-[2-[[4- | ¹H NMR (400 MHz, METHANOL- | 996.3 |
| | (8-isoquinolyl)-3-[2-[4-(8- | d4) δ ppm 9.02 (s, 1 H), 8.85 (s, | |
| | isoquinolyl)phenyl]ethoxy]phenyl]meth- | 1 H), 8.56-8.60 (m, 1 H), 8.43- | |
| | oxy]ethoxy]ethoxy]ethyl- | 8.48 (m, 1 H), 8.36 (d, J = 5.75 | |
| | carbamoyl]phenoxy]pyridine-3- | Hz, 1 H), 8.22 (dd, J = 8.63, 2.50 | |
| | carbonyl]amino]-5,5- | Hz, 1 H), 7.94 (dd, J = 8.25, 4.38 | |
| | dimethyl-hexanoic acid | Hz, 2 H), 7.89 (d, J = 5.38 Hz, 1 | |
| | | H), 7.79-7.86 (m, 3 H), 7.71 (d, | |
| | | J = 7.75 Hz, 1 H), 7.63 (d, J = 1.88 | |
| | | Hz, 1 H), 7.48 (dt, J = 11.35, 7.71 | |
| | | Hz, 3 H), 7.24-7.29 (m, 2 H), | |
| | | 7.17 (s, 1 H), 7.05-7.10 (m, 3 | |
| | | H), 7.00 (d, J = 8.76 Hz, 1 H), | |
| | | 6.79 (d, J = 7.75 Hz, 2 H), 4.62 (s, | |
| | | 2 H), 4.50 (dd, J = 8.63, 5.38 Hz, | |
| | | 1 H), 4.22-4.29 (m, 1 H), 4.11- | |
| | | 4.18 (m, 1 H), 3.64-3.72 (m, 10 | |
| | | H), 3.54-3.57 (m, 2 H), 2.71- | |
| | | 2.84 (m, 2 H), 1.90-2.01 (m, 1 | |
| | | H), 1.72-1.86 (m, 1 H), 1.29- | |
| | | 1.36 (m, 2 H), 0.89 (s, 9 H). | |
| BF035 | (2S)-2-[[6-[3-[2-[2-[2-[2- | ¹H NMR (400 MHz, METHANOL- | 1040.8 |
| | [[4-(8-isoquinolyl)-3-[2-[4-(8- | d4) δ ppm 9.03 (s, 1 H), 8.85 (s, | |
| | isoquinolyl)phenyl]ethoxy]phenyl]meth- | 1 H), 8.60 (d, J = 2.25 Hz, 1 H), | |
| | oxy]ethoxy]ethoxy]ethoxy]ethyl- | 8.46 (d, J = 5.88 Hz, 1 H), 8.36 (d, | |
| | carbamoyl]phenoxy]pyridine-3- | J = 5.75 Hz, 1 H), 8.24 (dd, | |
| | carbonyl]amino]-5,5- | J = 8.63, 2.38 Hz, 1 H), 7.94 (dd, | |
| | dimethyl-hexanoic acid | J = 7.82, 5.32 Hz, 2 H), 7.89 (d, | |
| | | J = 5.50 Hz, 1 H), 7.79-7.87 (m, | |
| | | 3 H), 7.71 (d, J = 7.50 Hz, 1 H), | |
| | | 7.61-7.64 (m, 1 H), 7.46-7.53 | |
| | | (m, 3 H), 7.25-7.31 (m, 2 H), | |
| | | 7.19 (s, 1 H), 7.05-7.12 (m, 3 | |
| | | H), 7.02 (d, J = 8.76 Hz, 1 H), 6.79 | |
| | | (d, J = 7.75 Hz, 2 H), 4.64 (s, 2 | |
| | | H), 4.50 (dd, J = 8.69, 5.19 Hz, 1 | |
| | | H), 4.24-4.31 (m, 1 H), 4.12- | |
| | | 4.19 (m, 1 H), 3.69 (s, 4 H), 3.61- | |
| | | 3.65 (m, 10 H), 3.51-3.55 (m, | |
| | | 2 H), 2.72-2.85 (m, 2 H), 1.90- | |
| | | 2.01 (m, 1 H), 1.75-1.86 (m, 1 | |
| | | H), 1.29-1.38 (m, 2 H), 0.89 (s, | |
| | | 9 H). | |
| BF037 | (2S)-2-[[6-[3-[2-[2-[2-[2- | ¹H NMR (400 MHz, METHANOL- | 1083.5 |
| | [2-[[4-(8-isoquinolyl)-3-[2-[4-(8- | d4) δ = 9.03 (s, 1H), 8.86 (s, 1H), | |
| | isoquinolyl)phenyl]ethoxy]phenyl]meth- | 8.60 (d, J = 2.4 Hz, 1H), 8.46 (d, | |
| | oxy]ethoxy]ethoxy]ethoxy]eth- | J = 5.8 Hz, 1H), 8.36 (d, J = 5.8 | |
| | oxy]ethylcarbamoyl]phenoxy]pyridine- | Hz, 1H), 8.24 (dd, J = 2.4, 8.6 | |
| | 3-carbonyl]amino]-5,5- | Hz, 1H), 7.94 (dd, J = 5.5, 8.1 | |
| | dimethyl-hexanoic acid | Hz, 2H), 7.90 (d, J = 5.8 Hz, 1H), | |
| | | 7.87-7.79 (m, 3H), 7.73-7.69 | |
| | | (m, 1H), 7.62 (t, J = 1.9 Hz, 1H), | |
| | | 7.53-7.46 (m, 3H), 7.31-7.25 | |
| | | (m, 2H), 7.21 (s, 1H), 7.13-7.01 | |
| | | (m, 4H), 6.79 (d, J = 7.9 Hz, 2H), | |
| | | 4.66 (s, 2H), 4.50 (dd, J = 5.1, | |
| | | 8.7 Hz, 1H), 4.28 (td, J = 5.7, 9.3 | |
| | | Hz, 1H), 4.17 (dt, J = 5.3, 8.2 Hz, | |
| | | 1H), 3.74-3.67 (m, 4H), 3.66- | |
| | | 3.55 (m, 14H), 3.54-3.50 (m, | |
| | | 2H), 2.86-2.70 (m, 2H), 2.01- | |
| | | 1.89 (m, 1H), 1.86-1.74 (m, | |
| | | 1H), 1.40-1.27 (m, 2H), 0.90 (s, | |
| | | 9H). | |
| BF044 | (2S)-2-[[6-[3-[2-[2-[2-[2- | ¹H NMR (400 MHz, METHANOL- | 1128.4 |
| | [2-[2-[[4-(8-isoquinolyl)-3-[2-[4-(8- | d4) δ ppm 9.03 (s, 1 H), 8.85 (s, | |
| | isoquinolyl)phenyl]ethoxy]phenyl]meth- | 1 H), 8.61 (d, J = 2.38 Hz, 1 H), | |
| | oxy]ethoxy]ethoxy]ethoxy]eth- | 8.46 (d, J = 5.96 Hz, 1 H), 8.36 (d, | |
| | oxy]ethoxy]ethylcarbamoyl]phen- | J = 5.48 Hz, 1 H), 8.22-8.27 (m, | |
| | oxy]pyridine-3-carbonyl]amino]-5,5- | 1 H), 7.91-7.97 (m, 2 H), 7.89 | |
| | dimethyl-hexanoic acid | (d, J = 6.20 Hz, 1 H), 7.84 (t, | |
| | | J = 7.03 Hz, 3 H), 7.72 (d, J = 8.46 | |
| | | Hz, 1 H), 7.63 (d, J = 1.67 Hz, 1 | |
| | | H), 7.50 (t, J = 7.99 Hz, 3 H), 7.27- | |
| | | 7.32 (m, 2 H), 7.21 (s, 1 H), | |
| | | 7.06-7.13 (m, 3 H), 7.04 (d, | |
| | | J = 8.58 Hz, 1 H), 6.80 (d, J = 8.11 | |
| | | Hz, 2 H), 4.67 (s, 2 H), 4.47- | |
| | | 4.53 (m, 1 H), 4.25-4.33 (m, 1 | |
| | | H), 4.13-4.22 (m, 1 H), 3.72 (s, | |
| | | 4 H), 3.52-3.67 (m, 20 H), 2.67- | |
| | | 2.91 (m, 2 H), 1.88-2.02 (m, 1 | |
| | | H), 1.72-1.87 (m, 1 H), 1.30- | |
| | | 1.37 (m, 2 H), 0.90 (s, 9 H). | |

### BF043

### 1. General Procedure for Preparation of tert-butyl N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]carbamate

To a solution of 3-[[2,2-dimethyl-6-[2-[(3R)-3-methylpiperazin-1-yl]pyrimidin-5-yl]-3-oxo-pyrrolo[2,3-b]pyridin-1-yl]methyl]pyridine-2-carbonitrile (500 mg, 879.43 μmol, 1 eq, TFA), 2-(tert-butoxycarbonylamino) acetic acid (184.87 mg, 1.06 mmol, 1.2 eq) and HATU (501.58 mg, 1.32 mmol, 1.5 eq) in DMF (3 mL) was added DIEA (340.98 mg, 2.64 mmol, 459.54 uL, 3 eq) dropwise at 0° C. Then the mixture was stirred at 20° C. for 2 hr. The mixture was diluted with water 20 mL, and extracted with Ethyl acetate 60 mL (20 mL*3). The combined organic layers were washed with brine 50 mL, dried over Na₂SO₄, filtered and concentrated under reduced pressure to give a residue which was purified by flash silica gel chromatography (ISCO®; 12 g SepaFlash® Silica Flash Column, Eluent of 0˜75% Ethyl acetate/Petroleum ether gradient @ 40 mL/min) to give tert-butyl N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]carbamate (450 mg, 735.66 umol, 83.65% yield) as a yellow oil.

### Data:

LCMS (ESI⁺): m/z 612.2 (M+H)

### 2. General Procedure for Preparation of 3-[[6-[2-[(3R)-4-(2-aminoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile

A solution of tert-butyl N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]carbamate (450 mg, 735.66 μmol, 1 eq) in DCM (5 mL) and TFA (1 mL) was stirred at 20° C. for 1 hr. The DCM and TFA was removed under reduced pressure to give 3-[[6-[2-[(3R)-4-(2-aminoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (460 mg, crude, TFA) as a yellow oil.

### Data:

LCMS (ESI⁺): m/z 512.2 (M+H)

### 3. General Procedure for Preparation of tert-butyl N-[10-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-10-oxo-decyl]carbamate

To a solution of 10-(tert-butoxycarbonylamino) decanoic acid (45.94 mg, 159.85 μmol, 1 eq), 3-[[6-[2-[(3R)-4-(2-aminoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (100 mg, 159.85 umol, 1 eq, TFA) in DMF (2 mL) was added DIEA (61.98 mg, 479.54 μmol, 83.53 uL, 3 eq) and HATU (91.17 mg, 239.77 μmol, 1.5 eq) at 0° C. The mixture was stirred at 20° C. for 1 hr. The mixture was diluted with water 5 mL, and then extracted with Ethyl acetate 15 mL (5 mL*3). The combined organic layers were washed with brine 10 mL, dried over Na₂SO₄, filtered and concentrated under reduced pressure to give a residue which was purified by prep-TLC (SiO₂, Ethyl acetate: Petroleum ether=10:1) to give tert-butyl N-[10-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-10-oxo-decyl]carbamate (70 mg, 89.63 μmol, 56.07% yield) as a yellow oil.

### Data:

LCMS (ESI⁺): m/z 781.5 (M+H)

### 4. General Procedure for Preparation of 10-amino-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]decanamide

A solution of tert-butyl N-[10-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-10-oxo-decyl]carbamate (70 mg, 89.63 μmol, 1 eq) in DCM (1 mL) and TFA (0.2 mL) was stirred at 20° C. for 1 hr. The DCM and TFA was removed under reduced pressure to give 10-amino-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]decanamide (70 mg, crude, TFA) as a yellow oil.

### Data:

LCMS (ESI⁺): m/z 681.5 (M+H)

### 5. General Procedure for Preparation of methyl (2S)-2-[[6-[3-[[10-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-10-oxo-decyl]carbamoyl]phenoxy]pyridine-3-carbonyl]amino]-5,5-dimethyl-hexanoate

To a solution of 10-amino-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]decanamide (70 mg, 88.07 μmol, 1 eq, TFA), 3-[[5-[[(1S)-1-methoxycarbonyl-4,4-dimethyl-pentyl]carbamoyl]-2-pyridyl]oxy]benzoic acid (36.50 mg, 69.06 μmol, 7.84e-1 eq, TFA) and DIEA (34.15 mg, 264.20 μmol, 46.02 uL, 3 eq) in DMF (2 mL) was added HATU (50.23 mg, 132.10 μmol, 1.5 eq) dropwise at 0° C. Then the mixture was stirred at 20° C. for 1 hr. The mixture was diluted with water 5 mL, and then extracted with Ethyl acetate 15 mL (5 mL*3). The combined organic layers were washed with brine 10 mL, dried over Na₂SO₄, filtered and concentrated under reduced pressure to give methyl (2S)-2-[[6-[3-[[10-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-10-oxo-decyl]carbamoyl]phenoxy]pyridine-3-carbonyl]amino]-5,5-dimethyl-hexanoate (90 mg, crude) as a yellow oil, which was used to next step without purification.

### Data:

LCMS (ESI⁺): m/z 1077.5 (M+H)

### 6. General Procedure for Preparation of (2S)-2-[[6-[3-[[[10-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-10-oxo-decyl]carbamoyl]phenoxy]pyridine-3-carbonyl]amino]-5,5-dimethyl-hexanoic acid

To a solution of methyl (2S)-2-[[6-[3-[[10-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-10-oxo-decyl]carbamoyl]phenoxy]pyridine-3-carbonyl]amino]-5,5-dimethyl-hexanoate (90 mg, 83.54 μmol, 1 eq) in THF (0.5 mL) and H₂O (0.5 mL) was added LiOH·H₂O (7.01 mg, 167.09 μmol, 2 eq). The mixture was stirred at 20° C. for 1 hr. The THF was removed under reduced pressure and the residue acid with FA to pH=2 and then purified by prep-HPLC (FA condition; column: Phenomenex Luna C18 200*40 mm*10 um; mobile phase: [water (FA)-ACN]; B %: 40%-80%, 8 min) to give (2S)-2-[[6-[3-[[10-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-10-oxo-decyl]carbamoyl]phenoxy]pyridine-3-carbonyl]amino]-5,5-dimethyl-hexanoic acid (30.8 mg, 28.97 μmol, 34.67% yield, 100% purity) as a light yellow solid.

### Data:

LCMS (ESI⁺): m/z 1063.5

¹H NMR (400 MHZ, METHANOL-d4) δ ppm 8.95 (s, 2H), 8.59-8.64 (m, 2H), 8.50 (br t, J=6.02 Hz, 1H), 8.27 (dd, J=8.70, 2.50 Hz, 1H), 8.03 (dd, J=8.17, 1.25 Hz, 1H), 7.94 (d, J=7.99 Hz, 1H), 7.71 (d, J=7.87 Hz, 1H), 7.59-7.64 (m, 2H), 7.53 (t, J=7.93 Hz, 1H), 7.28-7.35 (m, 2H), 7.07 (d, J=8.70 Hz, 1H), 5.12 (s, 2H), 4.61-4.78 (m, 3H), 4.51 (dd, J=8.82, 5.13 Hz, 1H), 4.21-4.42 (m, 1H), 3.97-4.20 (m, 2H), 3.74-3.84 (m, 0.5H) 3.42-3.51 (m, 0.5H), 3.34-3.39 (m, 2H), 3.15-3.28 (m, 1H), 3.01-3.15 (m, 1H), 2.27 (t, J=7.51 Hz, 2H), 1.91-2.01 (m, 1H), 1.75-1.87 (m, 1H), 1.55-1.68 (m, 4H), 1.33-1.40 (m, 16H), 1.21-1.33 (m, 3H), 1.10-1.20 (m, 2H), 0.92 (s, 9H).

The following compounds were prepared according to the general procedure.

The starting materials are either commercially available or may be prepared from commercially available reagents using conventional reactions well known in the art.

| | | | LC/MS |
|---|---|---|---|
| ID. | Name | HNMR | [M + 1] |
| BF042 | (2S)-2-[[6-[3-[[12-[[2-[(2R)-4-[5-[1-[(2- | ¹H NMR (400 MHz, | 1091.5 |
| | cyano-3-pyridyl)methyl]-2,2-dimethyl- | METHANOL-d4) δ | |
| | 3-oxo-pyrrolo[2,3-b]pyridin-6- | ppm 8.95 (s, 2 H), 8.60- | |
| | yl]pyrimidin-2-yl]-2-methyl-piperazin- | 8.64 (m, 2 H), 8.50 | |
| | 1-yl]-2-oxo-ethyl]amino]-12-oxo- | (br t, J = 5.66 Hz, 1 H), | |
| | dodecyl]carbamoyl]phenoxy]pyridine- | 8.27 (dd, J = 8.70, 2.50 | |
| | 3-carbonyl]amino]-5,5-dimethyl- | Hz, 1 H), 8.00-8.06 | |
| | hexanoic acid | (m, 1 H), 7.94 (d, | |
| | | J = 7.99 Hz, 1 H), 7.71 | |
| | | (d, J = 7.75 Hz, 1 H), | |
| | | 7.58-7.64 (m, 2 H), | |
| | | 7.53 (t, J = 7.93 Hz, 1 | |
| | | H), 7.28-7.36 (m, 2 | |
| | | H), 7.07 (d, J = 8.58 Hz, | |
| | | 1 H), 5.12 (s, 2 H), | |
| | | 4.58-4.78 (m, 3 H), | |
| | | 4.51 (dd, J = 8.94, 5.13 | |
| | | Hz, 1 H), 4.21-4.42 | |
| | | (m, 1 H), 3.96-4.21 | |
| | | (m, 2 H), 3.69-3.85 | |
| | | (m, 0.5 H), 3.48 (br dd, | |
| | | J = 3.16, 1.61 Hz, 0.5 | |
| | | H), 3.34-3.37 (m, 2 | |
| | | H), 3.17-3.28 (m, 1 | |
| | | H), 3.00-3.14 (m, 1 | |
| | | H), 2.27 (t, J = 7.57 Hz, | |
| | | 2 H), 1.90-2.02 (m, 1 | |
| | | H), 1.75-1.88 (m, 1 | |
| | | H), 1.58-1.65 (m, 4 | |
| | | H), 1.31 (br s, 20 H), | |
| | | 1.22-1.30 (m, 3 H), | |
| | | 1.10-1.19 (m, 2 H), | |
| | | 0.92 (s, 9 H). | |

### BF081

### 1. General Procedure for Preparation of tert-butyl 11-azidoundecanoate

To a solution of tert-butyl 11-bromoundecanoate (500 mg, 1.56 mmol, 1 eq) in DMF (8 mL) was added NaN₃ (150 mg, 2.31 mmol, 1.48 eq), then the reaction was stirred for 12 hr at 20° C. The reaction mixture was quenched by addition H₂O 20 mL, and then diluted with Na₂CO₃ 10 mL and extracted with EA 90 mL (30 mL*3). The combined organic layers were washed with brine 180 mL (90 mL*2), dried over Na₂SO₄, filtered and concentrated under reduced pressure to give tert-butyl 11-azidoundecanoate (440 mg, crude) as a light yellow oil, which was confirmed by H_NMR and used to next step without purification.

### Data:

¹H NMR (400 MHZ, CHLOROFORM-d) δ=3.41 (t, J=6.8 Hz, 2H), 2.20 (t, J=7.5 Hz, 2H), 1.86 (quin, J=7.2 Hz, 2H), 1.62-1.54 (m, 2H), 1.48-1.39 (m, 11H), 1.29 (s, 10H)

### 2. General Procedure for Preparation of methyl 6-(3-ethynylphenoxy)pyridine-3-carboxylate

To a solution of 3-ethynylphenol (950 mg, 8.04 mmol, 1 eq), methyl 6-fluoropyridine-3-carboxylate (1.25 g, 8.04 mmol, 1 eq) in DMF (15 mL) was added Cs₂CO₃ (3.93 g, 12.06 mmol, 1.5 eq). The mixture was stirred at 80° C. for 1 hr. The reaction mixture was partitioned between water 30 mL and EtOAc 30 mL. The organic phase was separated, washed with brine 70 mL (10 mL*7), dried over Na₂SO₄, filtered and concentrated under reduced pressure to give a residue. The residue was purified by flash silica gel chromatography (ISCO®; 40 g SepaFlash® Silica Flash Column, Eluent of 0˜10% Ethyl acetate/Petroleum ether gradient @ 120 mL/min) to give compound methyl 6-(3-ethynylphenoxy)pyridine-3-carboxylate (2 g, 7.90 mmol, 98.20% yield) as a white solid.

### Data:

LCMS (ESI⁺): m/z 254.1 (M+1)

### 3. General Procedure for Preparation of methyl 6-[3-[1-(11-tert-butoxy-11-oxo-undecyl)triazol-4-yl]phenoxy]pyridine-3-carboxylate

To a solution of the tert-butyl 11-azidoundecanoate (0.43 g, 1.52 mmol, 1 eq) and methyl 6-(3-ethynylphenoxy)pyridine-3-carboxylate (384.25 mg, 1.52 mmol, 1 eq) in DMSO (5 mL) was added cuprous; acetonitrile; hexafluorophosphate (1.58 g, 4.25 mmol, 2.8 eq). The reaction was stirred for 2 hr at 25° C. The reaction mixture was partitioned between water 20 mL and E^{A} 20 mL. The organic phase was separated, washed with brine 60 mL (10 mL*6), dried over Na₂SO₄, filtered and concentrated under reduced pressure to give a residue. The residue was purified by flash silica gel chromatography (ISCO®; 40 g SepaFlash® Silica Flash Column, Eluent of 0˜20% Ethyl acetate/Petroleum ether gradient @ 120 mL/min) to give compound methyl 6-[3-[1-(11-tert-butoxy-11-oxo-undecyl)triazol-4-yl]phenoxy]pyridine-3-carboxylate (0.8 g, 1.49 mmol, 98.25% yield) as a white solid.

### Data:

LCMS (ESI⁺): m/z 537.3 (M+1)

### 4. General Procedure for Preparation of 11-[4-[3-[(5-methoxycarbonyl-2-pyridyl)oxy]phenyl]triazol-1-yl]undecanoic acid

A mixture of methyl 6-[3-[1-(11-tert-butoxy-11-oxo-undecyl)triazol-4-yl]phenoxy]pyridine-3-carboxylate (0.8 g, 1.49 mmol, 1 eq) in TFA (0.2 mL) and DCM (1 mL) was stirred for 1 hr at 20° C. The mixture was concentrated under reduced pressure to give compound 11-[4-[3-[(5-methoxycarbonyl-2-pyridyl)oxy]phenyl]triazol-1-yl]undecanoic acid (0.8 g, 1.35 mmol, 90.26% yield, TFA) as a light yellow oil.

### Data:

LCMS (ESI⁺): m/z 481.3 (M+1)

### 5. General Procedure for Preparation of methyl 6-[3-[1-[11-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-11-oxo-undecyl]triazol-4-yl]phenoxy]pyridine-3-carboxylate

To a solution of 11-[4-[3-[(5-methoxycarbonyl-2-pyridyl)oxy]phenyl]triazol-1-yl]undecanoic acid (200 mg, 336.37 μmol, 1 eq, TFA), 3-[[6-[2-[(3R)-4-(2-aminoacetyl)-3-methyl-piperazin-1-yl]pyrimidin-5-yl]-2,2-dimethyl-3-oxo-pyrrolo[2,3-b]pyridin-1-yl]meth yl]pyridine-2-carbonitrile (210.44 mg, 336.37 μmol, 1 eq, TFA) in DMF (3 mL) was added DIEA (130.42 mg, 1.01 mmol, 175.77 uL, 3 eq) and HATU (191.85 mg, 504.56 μmol, 1.5 eq) at 0° C., then the reaction was stirred for 2 hr at 25° C. The reaction mixture was partitioned between water 10 mL and EA 10 mL. The organic phase was separated, washed with brine 30 mL (5 mL*6), dried over Na₂SO₄, filtered and concentrated under reduced pressure to give a residue. The residue was purified by flash silica gel chromatography (ISCO®; 12 g SepaFlash® Silica Flash Column, Eluent of 0˜90% Ethylacetate/Petroleum ether gradient @ 80 mL/min) to give compound methyl 6-[3-[1-[11-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-11-oxo-undecyl]triazol-4-yl]phenoxy]pyridine-3-carboxylate (170 mg, 174.52 μmol, 51.88% yield) as a yellow oil.

### Data:

LCMS (ESI⁺): m/z 974.5 (M+1)

### 6. General Procedure for Preparation of 6-[3-[1-[11-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-11-oxo-undecyl]triazol-4-yl]phenoxy]pyridine-3-carboxylic acid

To a solution of methyl 6-[3-[1-[11-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-11-oxo-undecyl]triazol-4-yl]phenoxy]pyridine-3-carboxylate (170 mg, 174.52 μmol, 1 eq) in THF (3 mL) and H₂O (1 mL) was added LiOH·H₂O (14.65 mg, 349.03 μmol, 2 eq). The mixture was stirred at 20° C. for 1 hr. The PH of residue was adjusted with HCl (1M) to 2. The reaction mixture was partitioned between water 5 mL and EA 5 mL. The organic phase was separated, washed with brine 15 mL (5 mL*3), dried over Na₂SO₄, filtered and concentrated under reduced pressure to give compound 6-[3-[1-[11-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-11-oxo-undecyl]triazol-4-yl]phenoxy]pyridine-3-carboxylic acid (0.2 g, crude) as a light yellow oil.

### Data:

LCMS (ESI⁺): m/z 960.4 (M+1)

### 7. General Procedure for Preparation of (2,5-dioxopyrrolidin-1-yl) 6-[3-[1-[11-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-11-oxo-undecyl]triazol-4-yl]phenoxy]pyridine-3-carboxylate

To a solution of 6-[3-[1-[11-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-11-oxo-undecyl]triazol-4-yl]phenoxy]pyridine-3-carboxylic acid (180 mg, 180.62 μmol, 1 eq, HCl), 1-hydroxypyrrolidine-2,5-dione (103.94 mg, 903.11 μmol, 5 eq) in DMF (2 mL) was added EDCI (173.13 mg, 903.11 μmol, 5 eq) at 0° C. The mixture was stirred at 25° C. for 1 hr. The reaction mixture was quenched by addition H₂O 4 mL, and extracted with EA 15 mL (5 mL*3), washed by brine 9 mL (3 mL*3). The combined organic layers were dried over Na₂SO₄, filtered and concentrated under reduced pressure to give compound (2,5-dioxopyrrolidin-1-yl) 6-[3-[1-[11-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-11-oxo-undecyl]triazol-4-yl]phenoxy]pyridine-3-carboxylate (190 mg, crude) as a yellow oil.

### Data:

LCMS (ESI⁺): m/z 1057.5 (M+1)

### 8. General Procedure for Preparation of General Procedure for Preparation of (2S)-2-[[6-[3-[1-[11-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-11-oxo-undecyl]triazol-4-yl]phenoxy]pyridine-3-carbonyl]amino]-5,5-dimethyl-hexanoic acid

To a solution of (2,5-dioxopyrrolidin-1-yl) 6-[3-[1-[11-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-11-oxo-undecyl]triazol-4-yl]phenoxy]pyridine-3-carboxylate (40 mg, 37.84 μmol, 1 eq), (2S)-2-amino-5,5-dimethyl-hexanoic acid (6.02 mg, 37.84 μmol, 1 eq) in DMF (0.6 mL),DCM (0.1 mL) and H₂O (0.3 mL) was added DIEA (9.78 mg, 75.67 μmol, 13.18 uL, 2 eq). The mixture was stirred at 25° C. for 1 hr. The mixture was filtered and the filtrate was purified by prep-HPLC. The residue was purified by prep-HPLC (FA condition column: column: Phenomenex Luna C18 200*40 mm*10 um; mobile phase: [water (FA)-ACN]; B %: 65%-90%, 8 min) to give compound (2S)-2-[[6-[3-[1-[11-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-11-oxo-undecyl]triazol-4-yl]phenoxy]pyridine-3-carbonyl]amino]-5,5-dimethyl-hexanoic acid (16.5 mg, 14.66 μmol, 7.75% yield, 97.85% purity) as a yellow solid.

### Data:

LCMS (ESI⁺): m/z 1101.5 (M+1)

¹H NMR (400 MHZ, METHANOL-d4) δ=8.94 (s, 2H), 8.64-8.61 (m, 2H), 8.37 (s, 1H), 8.26 (dd, J=2.5, 8.6 Hz, 1H), 8.03 (d, J=7.7 Hz, 1H), 7.94 (d, J=8.1 Hz, 1H), 7.72 (d, J=7.6 Hz, 1H), 7.65-7.59 (m, 2H), 7.51 (t, J=8.0 Hz, 1H), 7.29 (d, J=8.0 Hz, 1H), 7.14 (dd, J=2.2, 8.0 Hz, 1H), 7.07 (d, J=8.6 Hz, 1H), 5.11 (s, 2H), 4.75-4.60 (m, 3H), 4.51 (br d, J=3.7 Hz, 1H), 4.43 (t, J=7.0 Hz, 2H), 4.39-4.22 (m, 1H), 4.19-4.11 (m, 1H), 4.08-3.96 (m, 1H), 3.84-3.73 (m, 1H), 3.50-3.42 (m, 1H), 3.29-3.18 (m, 1H), 3.13-2.99 (m, 1H), 2.26 (t, J=7.5 Hz, 2H), 2.00-1.90 (m, 3H), 1.86-1.76 (m, 1H), 1.61 (br t, J=6.9 Hz, 2H), 1.37-1.29 (m, 20H), 1.26-1.11 (m, 3H), 0.92 (s, 9H)

### BF047, BF046, BF039, BF038

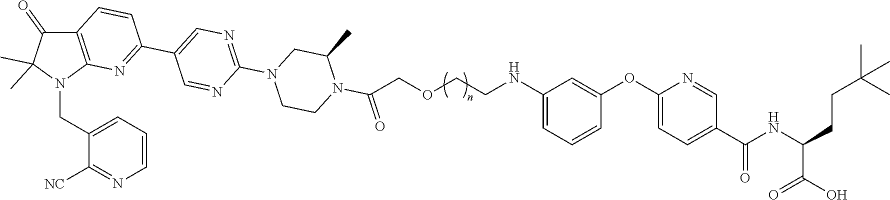

BF047 or ARG-DMX-C1 (n=1)
BF046 or ARG-DMX-C2 (n=3)
BF039 or ARG-DMX-C3 (n=5)
BF038 or ARG-DMX-C4 (n=7)

### General Information:

Reagents and solvents obtained from commercial suppliers were used without purification or drying unless otherwise noted. 1H NMR was recorded using AVNeo400-446284. TMS was used as an internal standard. LCMS analysis was performed on Acquity, HPLC with WATERS under the following conditions.

### LC-MS:

Column: Acquity UPLC BEH C18 1.7 μm, 50×2.1 mm; Mobile phase A: 0.1% Formic acid in water; Mobile phase B: 0.1% Formic acid in acetonitrile; Flow-0.6 mL/min; Temp: 40° C.

Time (min) % B: 0-5; 0.3-5; 2.5-95; 3.7-95; 4-5; 4.6-5

### HPLC:

Column: Acquity UPLC BEH C18, 1.7 μm, 50×2.1 mm; Mobile Phase A: 0.1% Formic Acid in water; Mobile Phase B: 100% Acetonitrile; Gradient (T % B): 0/3, 1.0/3, 7.0/95, 7.5/95, 9.0/3, 10/3 Flow Rate: 0.5 mL/min; Sample Diluent: Acetonitrile+water

### Prep HPLC Condition

Column/dimensions: X BRIDGE C18 5 μm, 19×250 mm; Mobile Phase A: 10 mM Ammonium Bicarbonate in water; Mobile Phase B: Acetonitrile; Gradient (Time/% B): 0/10, 2/10, 12/65, 15/65, 15.1/100, 17/100, 17.1/10, 20/10; Flow rate: 18 mL/min; Solubility: Acetonitrile+THF

### Chiral SFC Conditions

Analytical Column/dimensions: CHIRALPAK-AD 5 μm, 4.6×250 mm; % CO₂: 80%; % Co solvent: 20% (MeOH); Flow: 3.0 g/min; Back Pressure: 100 bar; Temperature: 30° C.; UV: 233 nm

Preparative SFC Conditions Column/dimensions: CHIRALPAK-AD-H 5 μm, 30×250 mm; % CO₂: 80%; % Co solvent: 20% (MeOH); Total Flow: 100 g/min; Back Pressure: 100 bar; Temperature: 30° C.; UV: 233 nm; Solubility: Acetonitrile+MeOH; No. of injections: 75; Total purification time: 6:00 h; Run time per injection: 06:00 min. Instrument details: Make/Model: SFC-150-I

### Experimental Procedure

### Step-1b

BF047 or ARG-DMX-1C (n=1): To a stirred solution of (R)-3-((6-(2-(4-(2-(2-aminoethoxy) acetyl)-3-methylpiperazin-1-yl)pyrimidin-5-yl)-2,2-dimethyl-3-oxo-2,3-dihydro-1H-pyrrolo[2,3-b]pyridin-1-yl)methyl) picolinonitrile (ARG-DMX-INT-1, n=1) (0.4 g, 0.72 mmol) and methyl(S)-2-(6-(3-iodophenoxy) nicotinamido)-5,5-dimethylhexanoate (ARG-DMX-BB—C) (0.32 g, 0.64 mmol) in 1,4-dioxane (8.0 mL) was added Cs₂CO₃ (0.47 g, 1.44 mmol). The reaction was de-gassed for 5 min with N₂ bubbling and Pd-PEPSI-iHeptCl (0.07 g, 0.072 mmol) was added. The reaction mixture was heated at 130° C. for 5 h. After completion of reaction (via TLC), the reaction mixture was allowed to cool at room temperature, quenched with ice water and extracted with ethyl acetate. The organic layer was dried over anhydrous Na₂SO₄, filtered and concentrated to afford crude compound. The resulting crude was purified by column chromatography (eluted with 70% ethyl acetate in pet ether) to afford methyl(S)-2-(6-(3-((2-(2-((R)-4-(5-(1-((2-cyanopyridin-3-yl)methyl)-2,2-dimethyl-3-oxo-2,3-dihydro-1H-pyrrolo[2,3-b]pyridin-6-yl)pyrimidin-2-yl)-2-methylpiperazin-1-yl)-2-oxoethoxy)ethyl)amino)phenoxy) nicotinamido)-5,5-dimethylhexanoate (ARG-DMX-1C, n=1) (0.25 g, 37%) as a pale yellow solid.

LCMS: m/z: 924.53 [M+H]⁺

BF046 or ARG-DMX-2C (n=3): To a stirred solution of (R)-3-((6-(2-(4-(2-(4-aminobutoxy) acetyl)-3-methylpiperazin-1-yl)pyrimidin-5-yl)-2,2-dimethyl-3-oxo-2,3-dihydro-1H-pyrrolo[2,3-b]pyridin-1-yl)methyl) picolinonitrile (ARG-DMX-INT-2, n=3) (0.18 g, 0.31 mmol) and methyl(S)-2-(6-(3-iodophenoxy) nicotinamido)-5,5-dimethylhexanoate (ARG-DMX-BB—C) (0.14 g, 0.28 mmol) in 1,4-dioxane (4.0 mL) was added Cs₂CO₃ (0.25 g, 0.77 mmol). The reaction was de-gassed for 5 min with N₂ bubbling and bis(tri-tert-butylphosphine) palladium (0.016 g, 0.031 mmol) was added. The reaction mixture was heated at 130° C. for 30 min. After completion of reaction (via TLC), the reaction mixture was allowed to cool up to room temperature, quenched with ice water and extracted with ethyl acetate. The organic layer was dried over anhydrous Na₂SO₄, filtered and concentrated to afford crude compound. The resulting crude was purified by column chromatography (eluted with 70% ethyl acetate in pet ether) to afford methyl (S)-2-(6-(3-((2-(2-((R)-4-(5-(1-((2-cyanopyridin-3-yl)methyl)-2,2-dimethyl-3-oxo-2,3-dihydro-1H-pyrrolo[2,3-b]pyridin-6-yl)pyrimidin-2-yl)-2-methylpiperazin-1-yl)-2-oxoethoxy)butyl)amino)phenoxy) nicotinamido)-5, 5-dimethylhexanoate (ARG-DMX-2C, n=3) (0.1 g, 34%) as pale yellow solid. LCMS: m/z: 952.96 [M+H]⁺

ARG-DMX-3C) (n=5) and ARG-DMX-4C) (n=7) were prepared in the same manner as described above for ARG-DMX-2C. The yields and analytical data are as follows,

ARG-DMX-3C (n=5) (0.05 g, 15%) as pale yellow solid. LCMS: m/z: 980.95 [M+H]⁺

ARG-DMX-4C (n=7) (0.05, 15% g) as a pale yellow solid. LCMS: m/z: 1009.15 [M+H]⁺

### Step-2a

ARG-DMX-C1 (n=1): Lithium hydroxide (0.032 g, 1.34 mmol) in water (0.5 mL) was dropwise added to a stirred solution of methyl(S)-2-(6-(3-((2-(2-((R)-4-(5-(1-((2-cyanopyridin-3-yl)methyl)-2,2-dimethyl-3-oxo-2,3-dihydro-1H-pyrrolo[2,3-b]pyridin-6-yl)pyrimidin-2-yl)-2-methylpiperazin-1-yl)-2-oxoethoxy)ethyl)amino)phenoxy) nicotinamido)-5,5-dimethylhexanoate (ARG-DMX-INT-1C, n=1) (0.25 g, 0.27 mmol) in THF (2.0 mL) at 0° C. The reaction was stirred at room temperature for 2 h. After completion of reaction (via TLC), the pH was adjusted between 5-6 using aq. 1.0 M HCl solution at 0° C. and extracted with ethyl acetate. The organic layer was dried over anhydrous Na₂SO₄, filtered and concentrated to afford crude compound. The crude was purified by prep HPLC to afford(S)-2-(6-(3-((2-(2-((R)-4-(5-(1-((2-cyanopyridin-3-yl)methyl)-2,2-dimethyl-3-oxo-2,3-dihydro-1H-pyrrolo[2,3-b]pyridin-6-yl)pyrimidin-2-yl)-2-methylpiperazin-1-yl)-2-oxoethoxy)ethyl)amino)phenoxy) nicotinamido)-5,5-dimethylhexanoic acid (ARG-DMX-C1) (n=1) (0.07 g, 28%) as a yellow solid

¹H NMR (400 MHZ, d6-DMSO) δ 8.94 (s, 2H), 8.65 (d, J=4.4 Hz, 1H), 8.61 (d, J=2.4 Hz, 1H) 8.46-8.36 (brs, 1H), 8.21 (dd, J=2.4, 8.8 Hz, 1H), 7.97 (d, J=8.4 Hz, 1H), 7.94 (d, J=8.0 Hz, 1H), 7.67 (dd, J=4.8, 8.0 Hz, 1H), 7.37 (d, J=8.0 Hz, 1H), 7.09 (t, J=8.0 Hz, 1H), 6.97 (d, J=8.6 Hz, 1H), 6.46 (d, J=8.1 Hz, 1H), 6.34 (t, J=2.0 Hz 1H), 6.27 (d, J=8.8 Hz, 1H), 5.93 (t, J=5.5 Hz, 1H), 5.04 (s, 2H), 4.77-4.40 (m, 3H), 4.36-4.08 (m, 4H), 3.66-3.54 (m, 2H), 3.26-3.18 (m, 2H), 3.00-2.85 (m, 1H), 1.83-1.60 (m, 2H), 1.31 (s, 6H), 1.28-0.97 (m, 6H), 0.83 (s, 9H). LCMS: m/z: 910.60 [M+H]⁺

Compounds BF046 (ARG-DMX-C2 (n=3)), BF039 (ARG-DMX-C3 (n=5)) and BF038 (ARG-DMX-C4 (n=7)) were prepared in the same manner as described above. The yields and analytical data are as follows,

BF046, ARG-DMX-C2 (n=3) (0.028 g, 38%) as a Yellow Solid

1H NMR (400 MHZ, d6-DMSO) δ 12.68 (s, 1H), 8.94 (s, 2H), 8.66 (d, J=3.6 Hz, 1H), 8.62 (d, J=2.0 Hz, 1H), 8.41 (brs, 1H), 8.22 (dd, J=2.4, 8.4 Hz, 1H), 7.96 (t, J=8.6 Hz, 2H), 7.67 (dd, J=4.4, 8.4 Hz, 1H), 7.37 (d, J=8.0 Hz, 1H), 7.09 (t, J=8.0 Hz, 1H), 6.97 (d, J=8.6 Hz, 1H), 6.43 (d, J=8.2 Hz, 1H), 6.30 (s, 1H), 6.24 (d, J=7.6 Hz, 1H), 5.80 (t, J=5.2 Hz, 1H), 5.04 (s, 2H), 4.78-3.67 (m, 7H), 3.50-3.40 (m, 2H), 3.05-2.96 (m, 2H), 1.88-1.51 (m, 6H), 1.31 (s, 6H), 1.28-0.98 (m, 7H), 0.84 (s, 9H). LCMS: m/z: 938.65 [M+H]⁺

BF039, ARG-DMX-C3 (n=5) (0.015 g, 30%) as a Yellow Solid

¹H NMR (400 MHZ, d6-DMSO) õ 8.94 (s, 2H), 8.66 (d, J=3.6 Hz, 1H), 8.62 (d, J=2.0 Hz, 1H) 8.28 (brs, 1H), 8.22 (dd, J=2.4, 8.4 Hz, 1H), 7.96 (t, J=8.9 Hz, 2H), 7.67 (dd, J=4.4, 8.4 Hz, 1H), 7.37 (d, J=8.0 Hz, 1H), 7.09 (t, J=8.0 Hz, 1H), 6.97 (d, J=8.6 Hz, 1H), 6.43 (d, J=8.2 Hz, 1H), 6.29 (s, 1H), 6.24 (d, J=7.6 Hz, 1H), 5.80 (brt, J=5.2 Hz, 1H), 5.04 (s, 2H), 4.76-3.68 (m, 7H), 3.48-3.37 (m, 2H), 3.00-2.89 (m, 2H), 1.85-1.60 (m, 2H), 1.57-1.44 (m, 4H), 1.40-0.98 (m, 15H), 0.83 (s, 9H). LCMS: m/z: 966.84 [M+H]⁺

BF038, ARG-DMX-C4 (n=7) (0.038 g, 76%) as a Yellow Solid

¹H NMR (400 MHZ, d6-DMSO) õ 12.71 (s, 1H), 8.94 (s, 2H), 8.66 (d, J=4.8 Hz, 1H), 8.62 (d, J=2.4 Hz, 1H), 8.47 (brs, 1H), 8.22 (dd, J=2.4, 8.8 Hz, 1H), 7.96 (t, J=8.8 Hz, 2H), 7.67 (dd, J=4.4, 8.0 Hz, 1H), 7.36 (d, J=8.0 Hz, 1H), 7.07 (t, J=8.0 Hz, 1H), 6.97 (d, J=8.6 Hz, 1H), 6.41 (d, J=8.2 Hz, 1H), 6.27 (s, 1H), 6.23 (d, J=8.4 Hz, 1H), 5.75 (t, J=5.2 Hz, 1H), 5.04 (s, 2H), 4.78-3.60 (m, 7H), 3.47-3.38 (m, 2H), 3.00-2.90 (m, 2H), 1.87-1.60 (m, 2H), 1.57-1.42 (m, 4H), 1.39-0.98 (m, 20H), 0.85 (s, 9H). LCMS (m/z: 994.75 [M+H]⁺

### BF064, BF048, BF036, BF040

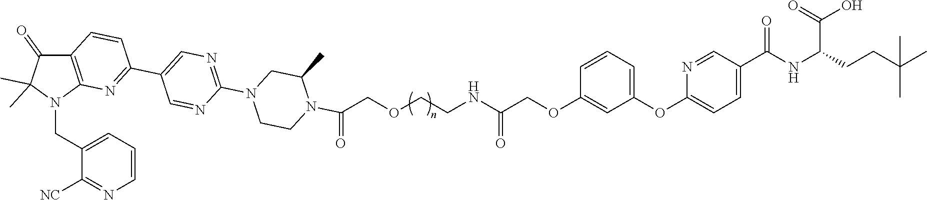

BF064 or ARG-DMX-B1 (n=1)
BF048 or ARG-DMX-B2 (n=3)
BF036 or ARG-DMX-B3 (n=5)
BF040 or ARG-DMX-B4 (n=7)

### General Information:

Reagents and solvents obtained from commercial suppliers were used without purification or drying unless otherwise noted. 1H NMR was recorded using AVNeo400-446284. TMS was used as an internal standard. LCMS analysis was performed on Acquity, HPLC with WATERS under the following conditions.

### LC-MS:

Column: Acquity UPLC BEH C18 1.7 μm, 50×2.1 mm; Mobile phase A: 0.1% Formic acid in water; Mobile phase B: 0.1% Formic acid in acetonitrile; Flow-0.6 mL/min; Temp: 40° C.

Time (min) % B: 0-5; 0.3-5; 2.5-95; 3.7-95; 4-5; 4.6-5

### HPLC:

Column: Acquity UPLC BEH C18, 1.7 μm, 50×2.1 mm; Mobile Phase A: 0.1% Formic Acid in water; Mobile Phase B: 100% Acetonitrile; Gradient (T % B): 0/3, 1.0/3, 7.0/95, 7.5/95, 9.0/3, 10/3 Flow Rate: 0.5 mL/min; Sample Diluent: Acetonitrile+water

### Prep HPLC Condition

Column/dimensions: X BRIDGE C18 5 μm, 19×250 mm; Mobile Phase A: 10 mM Ammonium Bicarbonate in water; Mobile Phase B: Acetonitrile; Gradient (Time/% B): 0/10, 2/10, 12/65, 15/65, 15.1/100, 17/100, 17.1/10, 20/10; Flow rate: 18 mL/min; Solubility: Acetonitrile+THF

### Chiral SFC Conditions

Analytical Column/dimensions: CHIRALPAK-AD 5 μm, 4.6×250 mm; % CO₂: 80%; % Co solvent: 20% (MeOH); Flow: 3.0 g/min; Back Pressure: 100 bar; Temperature: 30° C.; UV: 233 nm

Preparative SFC Conditions Column/dimensions: CHIRALPAK-AD-H 5 μm, 30×250 mm; % CO₂: 80%; % Co solvent: 20% (MeOH); Total Flow: 100 g/min; Back Pressure: 100 bar; Temperature: 30° C.; UV: 233 nm; Solubility: Acetonitrile+MeOH; No. of injections: 75; Total purification time: 6:00 h; Run time per injection: 06:00 min. Instrument details: Make/Model: SFC-150-I

### Synthetic Scheme for ARG-DMX-B1, B2, B3 & B4:

### Experimental Procedure

### Step-1a:

ARG-DMX-1B (n=1): HATU (0.04 mg, 0.11 mmol) was added to a stirred solution of (R)-3-((6-(2-(4-(2-(2-aminoethoxy) acetyl)-3-methylpiperazin-1-yl)pyrimidin-5-yl)-2,2-dimethyl-3-oxo-2,3-dihydro-1H-pyrrolo[2,3-b]pyridin-1-yl)methyl) picolinonitrile (ARG-DMX-INT-1, n=1) (0.04 g, 0.07 mmol), (S)-2-(3-((5-((1-methoxy-5,5-dimethyl-1-oxohexan-2-yl)carbamoyl)pyridin-2-yl)oxy)phenoxy) acetic acid (ARG-DMX-BB—B) (0.034 g, 0.08 mmol) and DIPEA (0.04 mL, 0.23 mmol) in N,N-dimethylformamide (1.0 mL) at 0° C. The reaction mixture was stirred at room temperature for 3 h. After completion of reaction (via TLC), the reaction mass was quenched with ice cold water and extracted with ethyl acetate. The organic layer was dried over anhydrous Na₂SO₄, filtered and concentrated to afford crude compound. The crude was purified by column chromatography (eluted with 70-100% ethyl acetate in pet ether) to afford methyl(S)-2-(6-(3-(2-((6-(2-((R)-4-(5-(1-((2-cyanopyridin-3-yl)methyl)-2,2-dimethyl-3-oxo-2,3-dihydro-1H-pyrrolo[2,3-b]pyridin-6-yl)pyrimidin-2-yl)-2-methylpiperazin-1-yl)-2-oxoethoxy)ethyl)amino)-2-oxoethoxy)phenoxy) nicotinamido)-5,5-dimethylhexanoate (ARG-DMX-1B) (n=1) (0.07 g, 71%) as a yellow gum.

LCMS: m/z: 982.56 [M+H]⁺

Compounds ARG-DMX-2B (n=3), ARG-DMX-3B (n=5) and ARG-DMX-4B (n=7) were prepared in the same manner as described above. The yields and analytical data are as follows,
ARG-DMX-2B (n=3) (0.07 g) as a yellow gum. LCMS: m/z: 1010.63 [M+H]⁺
ARG-DMX-3B (N=5) (0.06 g) as a yellow gum. LCMS: m/z: 1038.81 [M+H]⁺
ARG-DMX-4B (n=7) (0.04 g, 86%) as a yellow gum. LCMS: m/z: 1066.99 [M+H]⁺

### Step-2a

ARG-DMX-B1 (n=1): Lithium hydroxide (0.003 g, 0.13 mmol) in water (0.5 mL) was dropwise added to a stirred solution of methyl(S)-2-(6-(3-(2-((6-(2-((R)-4-(5-(1-((2-cyanopyridin-3-yl)methyl)-2,2-dimethyl-3-oxo-2,3-dihydro-1H-pyrrolo[2,3-b]pyridin-6-yl)pyrimidin-2-yl)-2-methylpiperazin-1-yl)-2-oxoethoxy)ethyl)amino)-2-oxoethoxy)phenoxy) nicotinamido)-5,5-dimethylhexanoate (ARG-DMX-1B) (n=1) (0.035 g, 0.06 mmol) in THF (2.0 mL) at 0° C. The reaction was stirred at room temperature for 2 h. After completion of reaction (via TLC), the pH was adjusted between 5-6 using aq. 1.0 M HCl solution at 0° C. and extracted with ethyl acetate. The organic layer was dried over anhydrous Na₂SO₄, filtered and concentrated to afford crude compound. The crude was purified by prep HPLC to afford(S)-2-(6-(3-(2-((8-(2-((R)-4-(5-(1-((2-cyanopyridin-3-yl)methyl)-2,2-dimethyl-3-oxo-2,3-dihydro-1H-pyrrolo[2,3-b]pyridin-6-yl)pyrimidin-2-yl)-2-methylpiperazin-1-yl)-2-oxoethoxy)methyl)amino)-2-oxoethoxy)phenoxy) nicotinamido)-5,5-dimethylhexanoic acid (ARG-DMX-B1) (n=1) (0.023 g, 58%) as a yellow solid.

1H NMR (400 MHZ, d6-DMSO) δ 12.62 (s, 1H), 8.93 (s, 2H), 8.70-8.61 (m, 3H), 8.40-8.26 (brs+dd, J=4.4, 8.8 Hz, 2H), 7.96 (t, J=8.5 Hz, 2H), 7.67 (dd, J=4.4, 8.0 Hz, 1H), 7.36 (d, J=8.0 Hz, 1H), 7.32 (d, J=8.0 Hz, 1H), 7.08 (d, J=8.6 Hz, 1H), 6.90-6.82 (m, 2H), 6.76 (d, J=7.9 Hz, 1H), 5.05 (s, 2H), 4.70-4.40 (m, 4H), 4.37-4.08 (m, 4H), 3.59-3.46 (m, 2H), 3.00-2.84 (m, 1H), 1.86-1.62 (m, 2H), 1.31 (s, 6H), 1.29-0.95 (m, 8H), 0.85 (s, 9H). LCMS: m/z: 968.56 [M+H]⁺

Compounds BF048 (ARG-DMX-B2 (n=3)), BF036 (ARG-DMX-B3 (n=5)) and BF040 (ARG-DMX-B4 (n=7)) were prepared in the same manner as described above. The yields and analytical data are as follows;

BF048, ARG-DMX-B2 (n=3) (0.018 g, 24%) as a Yellow Solid

1H NMR (400 MHZ, d6-DMSO) δ 12.58 (s, 1H), 8.94 (s, 2H), 8.67 (d, J=3.4 Hz, 1H), 8.59 (d, J=2.0 Hz, 1H), 8.35-8.20 (brs+dd, J=2.4, 8.8 Hz, 2H), 8.10 (t, J=5.8 Hz, 1H), 7.96 (t, J=9.1 Hz, 2H), 7.67 (dd, J=4.4, 8.0 Hz, 1H), 7.36 (d, J=8.0 Hz, 1H), 7.32 (t, J=8.0 Hz, 1H), 7.06 (d, J=8.6 Hz, 1H), 6.83 (dd, J=2.0, 8.4 Hz, 1H), 6.79-6.73 (m, 2H), 5.04 (s, 2H), 4.76-4.40 (m, 4H), 4.30-4.01 (m, 4H), 3.48-3.37 (m, 2H), 3.19-3.08 (m, 2H), 3.01-2.87 (m, 1H), 1.85-1.59 (m, 2H), 1.56-1.41 (m, 4H), 1.31 (s, 6H), 1.26-1.00 (m, 6H), 0.82 (s, 9H). LCMS: m/z: 996.68 [M+H]⁺

BF036, ARG-DMX-B3 (n=5) (0.025 g, 42%) as a Yellow Solid

¹H NMR (400 MHZ, d6-DMSO) δ 12.58 (s, 1H), 8.94 (s, 2H), 8.67 (d, J=3.4 Hz, 1H), 8.61 (d, J=2.0 Hz, 1H), 8.47-8.33 (brs, 1H), 8.25 (dd, J=2.4, 8.8 Hz, 1H), 8.06 (t, J=5.7 Hz, 1H), 7.96 (t, J=8.8 Hz, 2H), 7.67 (dd, J=4.4, 8.0 Hz, 1H), 7.36 (d, J=8.0 Hz, 1H), 7.32 (d, J=8.4 Hz, 1H), 7.07 (d, J=8.6 Hz, 1H), 6.84 (dd, J=2.0, 8.4 Hz, 1H), 6.77-6.70 (m, 2H), 5.04 (s, 2H), 4.74-4.09 (m, 8H), 3.42-3.37 (m, 2H), 3.13-3.05 (m, 2H), 1.86-1.60 (m, 2H), 1.53-1.34 (m, 4H), 1.27-1.06 (m, 16H), 0.84 (s, 9H). LCMS: m/z: 1022.72 [M+H]⁺

BF040, ARG-DMX-B4 (n=7) (0.022 g, 44%) as a Yellow Solid

1H NMR (400 MHZ, d6-DMSO) õ 8.94 (s, 2H), 8.67 (d, J=3.4 Hz, 1H), 8.59 (s, 1H), 8.30-8.12 (brs+d, J=4.2 Hz, 2H), 8.05 (t, J=5.6 Hz, 1H), 7.96 (t, J=8.8 Hz, 2H), 7.67 (dd, J=4.8, 8.0 Hz, 1H), 7.36 (d, J=8.0 Hz, 1H), 7.32 (t, J=8.4 Hz, 1H), 7.06 (d, J=8.6 Hz, 1H), 6.83 (d, J=8.8 Hz, 1H), 6.79-6.72 (m, 2H), 5.04 (s, 2H), 4.75-3.96 (m, 8H), 3.43-3.35 (m, 2H), 3.12-3.02 (m, 2H), 2.79-2.68 (m, 1H), 1.85-1.73 (m, 1H), 1.70-1.59 (m, 1H), 1.55-1.46 (m, 2H), 1.44-1.35 (m, 2H), 1.31 (s, 6H), 1.25-1.00 (m, 14H), 0.82 (s, 9H). LCMS: m/z: 1052.53 [M+H]⁺

### BF058, BF045, BF044, BF041

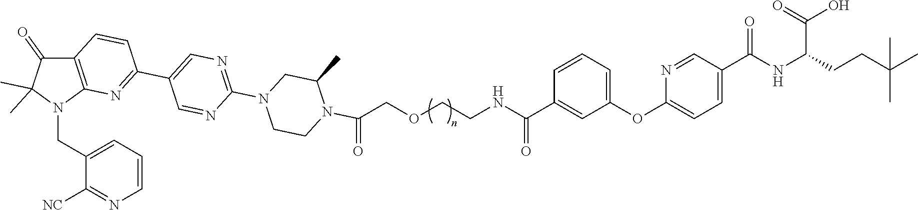

BF058 or ARG-DMX-A1 (n=1)
BF045 or ARG-DMX-A2 (n=3)
BF044 or ARG-DMX-A3 (n=5)
BF041 or ARG-DMX-A4 (n=7)

### General Information

Reagents and solvents obtained from commercial suppliers were used without purification or drying unless otherwise noted. 1H NMR was recorded using AVNeo400-446284. TMS was used as an internal standard. LCMS analysis was performed on Acquity, HPLC with WATERS under the following conditions.

### LC-MS:

Column: Acquity UPLC BEH C18 1.7 μm, 50×2.1 mm; Mobile phase A: 0.1% Formic acid in water; Mobile phase B: 0.1% Formic acid in acetonitrile; Flow-0.6 mL/min; Temp: 40° C.

Time (min) % B: 0-5; 0.3-5; 2.5-95; 3.7-95; 4-5; 4.6-5

### HPLC:

Column: Acquity UPLC BEH C18, 1.7 μm, 50×2.1 mm; Mobile Phase A: 0.1% Formic Acid in water; Mobile Phase B: 100% Acetonitrile; Gradient (T % B): 0/3, 1.0/3, 7.0/95, 7.5/95, 9.0/3, 10/3 Flow Rate: 0.5 mL/min; Sample Diluent: Acetonitrile+water

### Prep HPLC Condition

Column/dimensions: X BRIDGE C18 5 μm, 19×250 mm; Mobile Phase A: 10 mM Ammonium Bicarbonate in water; Mobile Phase B: Acetonitrile; Gradient (Time/% B): 0/10, 2/10, 12/65, 15/65, 15.1/100, 17/100, 17.1/10, 20/10; Flow rate: 18 mL/min; Solubility: Acetonitrile+THF

### Chiral SFC Conditions

Analytical Column/dimensions: CHIRALPAK-AD 5 μm, 4.6×250 mm; % CO₂: 80%; % Co solvent: 20% (MeOH); Flow: 3.0 g/min; Back Pressure: 100 bar; Temperature: 30° C.; UV: 233 nm

Preparative SFC Conditions Column/dimensions: CHIRALPAK-AD-H 5 μm, 30×250 mm; % CO₂: 80%; % Co solvent: 20% (MeOH); Total Flow: 100 g/min; Back Pressure: 100 bar; Temperature: 30° C.; UV: 233 nm; Solubility: Acetonitrile+MeOH; No. of injections: 75; Total purification time: 6:00 h; Run time per injection: 06:00 min. Instrument details: Make/Model: SFC-150-I

### Sideskift

### Synthetic Scheme for ARG-DMX-A1, A2, A3 & A4:

### Experimental Procedure

### Step-1

ARG-DMX-1A (n=1): To a stirred solution of (R)-3-((6-(2-(4-(2-(2-aminoethoxy) acetyl)-3-methylpiperazin-1-yl)pyrimidin-5-yl)-2,2-dimethyl-3-oxo-2,3-dihydro-1H-pyrrolo[2,3-b]pyridin-1-yl)methyl) picolinonitrile (ARG-DMX-INT-1, n=1) (0.04 g, 0.07 mmol), (S)-3-((5-((1-methoxy-5,5-dimethyl-1-oxohexan-2-yl)carbamoyl)pyridin-2-yl)oxy)benzoic acid (ARG-DMX-BB-A) (0.034 g, 0.08 mmol) and DIPEA (0.04 mL, 0.23 mmol) in N,N-dimethylformamide (1.0 mL) at 0° C. was added HATU (0.04 g, 0.11 mmol). The reaction mixture was stirred at room temperature for 3 h. After completion of reaction (via TLC), the reaction mixture was quenched with ice cold water and extracted with ethyl acetate. The organic layer was dried over anhydrous Na₂SO₄, filtered and concentrated to afford crude. The crude was purified by column chromatography (eluted with 70-100% ethyl acetate in pet ether) to afford(S)-2-(6-(3-((2-(2-((R)-4-(5-(1-((2-cyanopyridin-3-yl)methyl)-2,2-dimethyl-3-oxo-2,3-dihydro-1H-pyrrolo[2,3-b]pyridin-6-yl)pyrimidin-2-yl)-2-methylpiperazin-1-yl)-2-oxoethoxy)ethyl)carbamoyl)phenoxy) nicotinamido)-5,5-dimethylhexanoic acid (ARG-DMX-1A, n=1) (0.05 g, 70%) as a yellow gum.

LCMS: (m/z): 952.65 [M+H]⁺

Compounds ARG-DMX-2A (n=3), ARG-DMX-3A (n=5) and ARG-DMX-4A (n=7) were prepared in the same manner as described above. The yields and analytical data are as follows;
ARG-DMX-2A (n=3) (0.07 g, 42%) as a yellow gum. LCMS: m/z: 980.61 [M+H]⁺
ARG-DMX-3A (n=5) (0.06 g, 78%) as a yellow gum. LCMS: m/z: 1008.79 [M+H]⁺
ARG-DMX-4A (n=7) (0.04 g, 48%) as a yellow gum. LCMS: m/z: 1036.92 [M+H]⁺

### Step-2:

ARG-DMX-A1 (n=1): Lithium hydroxide (0.003 g, 0.13 mmol) in water (0.5 mL) was added dropwise to a stirred solution of methyl(S)-2-(6-(3-((2-(2-((R)-4-(5-(1-((2-cyanopyridin-3-yl)methyl)-2,2-dimethyl-3-oxo-2,3-dihydro-1H-pyrrolo[2,3-b]pyridin-6-yl)pyrimidin-2-yl)-2-methylpiperazin-1-yl)-2-oxoethoxy)ethyl)carbamoyl)phenoxy) nicotinamido)-5,5-dimethyl hexanoate (ARG-DMX-1A, n=1) (0.04 g, 0.04 mmol) in THF (2.0 mL) at 0° C. The reaction was stirred at room temperature for 2 h. After completion of reaction (via TLC), the pH was adjusted between 5-6 using 1M aq. HCl at 0° C. and extracted with ethyl acetate. The organic layer was dried over anhydrous Na₂SO₄, filtered and concentrated to afford crude compound. The crude was purified by prep HPLC to afford(S)-2-(6-(3-((2-(2-((R)-4-(5-(1-((2-cyanopyridin-3-yl)methyl)-2,2-dimethyl-3-oxo-2,3-dihydro-1H-pyrrolo[2,3-b]pyridin-6-yl)pyrimidin-2-yl)-2-methylpiperazin-1-yl)-2-oxoethoxy)ethyl)carbamoyl)phenoxy) nicotinamido)-5,5-dimethyl hexanoic acid (ARG-DMX-A1, n=1) (0.015 g, 38%) as a yellow solid.

1H NMR (400 MHZ, d6-DMSO) δ 12.51 (s, 1H), 8.94 (s, 2H), 8.77-8.72 (brs, 1H), 8.67 (d, J=3.2 Hz, 1H), 8.60 (d, J=2.1 Hz, 1H), 8.47-8.34 (brs, 1H), 8.27 (dd, J=2.4, 8.8 Hz, 1H), 7.96 (t, J=8.8 Hz, 2H), 7.77 (d, J=7.8 Hz, 1H), 7.67 (t, J=6.3 Hz, 2H), 7.52 (t, J=7.9 Hz, 1H), 7.40-7.30 (m, 2H), 7.14 (d, J=8.6 Hz, 1H), 5.05 (s, 2H), 4.74-4.09 (m, 7H), 3.65-3.57 (m, 2H), 3.49-3.40 (m, 2H), 3.00-2.83 (m, 1H), 1.85-1.60 (m, 2H), 1.31 (s, 6H), 1.28-0.92 (m, 6H), 0.83 (s, 9H). LCMS: m/z: 938.50 [M+H]⁺

Compounds BF045 (ARG-DMX-A2 (n=3)), BF044 (ARG-DMX-A3 (n=5)) and BF041 (ARG-DMX-A4 (n=7) were prepared in the same manner as described above. The yields and analytical data are as follows;

BF045, ARG-DMX-A2 (n=3) (0.027 g, 39%) as a Yellow Solid

1H NMR (400 MHZ, d6-DMSO) δ 8.94 (s, 2H), 8.67 (d, J=3.6 Hz, 1H), 8.59 (d, J=2.1 Hz, 1H), 8.50 (t, J=5.5 Hz, 1H), 8.44-8.33 (brs, 1H), 8.26 (dd, J=2.4, 8.4 Hz, 1H), 7.96 (t, J=8.9 Hz, 2H), 7.72 (d, J=8.0 Hz, 1H), 7.67 (dd, J=4.4, 8.0 Hz, 1H), 7.62 (s, 1H), 7.50 (t, J=8.9 Hz, 1H), 7.37 (d, J=8.0 Hz, 1H), 7.32 (dd, J=1.6, 8.0 Hz, 1H), 7.13 (d, J=8.6 Hz, 1H), 5.04 (s, 2H), 4.75-4.05 (m, 6H), 3.50-3.40 (m, 2H), 3.05-2.84 (m, 1H), 1.85-1.50 (m, 6H), 1.31 (s, 6H), 1.28-0.96 (m, 6H), 0.83 (s, 9H). LCMS: m/z: 966.71 [M+H]⁺

BF044, ARG-DMX-A3 (n=5) (0.025 g, 42%) as a Yellow Solid

1H NMR (400 MHZ, d6-DMSO) õ 8.94 (s, 2H), 8.67 (d, J=3.4 Hz, 1H), 8.59 (d, J=2.0 Hz, 1H), 8.48 (t, J=5.5 Hz, 1H), 8.45-8.36 (brs, 1H), 8.27 (dd, J=2.4, 8.4 Hz, 1H), 7.96 (t, J=9.1 Hz, 2H), 7.72 (d, J=7.8 Hz, 1H), 7.67 (dd, J=4.4, 8.0 Hz, 1H), 7.62 (s, 1H), 7.51 (t, J=7.9 Hz, 1H), 7.37 (d, J=8.0 Hz, 1H), 7.32 (d, J=1.6, 8.0 Hz, 1H), 7.14 (d, J=8.6 Hz, 1H), 5.04 (s, 2H), 4.66-4.13 (m, 6H), 3.47-3.39 (m, 2H), 3.27-3.20 (m, 2H), 3.02-2.82 (m, 1H), 1.85-1.43 (m, 6H), 1.31-1.03 (m, 16H), 0.84 (s, 9H). LCMS: m/z: 994.78 [M+H]⁺

BF041, ARG-DMX-A4 (n=7) (0.011 g, 25%) as a Yellow Solid

1H NMR (400 MHZ, d6-DMSO) δ 8.94 (s, 2H), 8.67 (d, J=3.4 Hz, 1H), 8.58 (d, J=1.6 Hz, 1H), 8.47 (t, J=5.5 Hz, 1H), 8.39-8.20 (brs+d, J=3.5 Hz, 2H), 7.95 (t, J=8.4 Hz, 2H), 7.72 (d, J=7.8 Hz, 1H), 7.67 (dd, J=4.8, 8.0 Hz, 1H), 7.61 (s, 1H), 7.51 (t, J=7.9 Hz, 1H), 7.37 (d, J=8.0 Hz, 1H), 7.32 (dd, J=1.6, 8.0 Hz, 1H), 7.13 (d, J=8.5 Hz, 1H), 5.04 (s, 2H), 4.70-4.02 (m, 6H), 3.45-3.36 (m, 2H), 3.28-3.20 (m, 2H), 3.00-2.70 (m, 3H), 1.88-1.39 (m, 6H), 1.36-0.95 (m, 20H), 0.83 (s, 9H). LCMS: m/z: 1022.81 [M+H]⁺

### BF083, BF084, BF086

### 1. General Procedure for Preparation of methyl (2S)-5,5-dimethyl-2-[[6-[3-[2-oxo-2-[2-(2-prop-2-ynoxyethoxy)ethylamino]ethoxy]phenoxy]pyridine-3-carbonyl]amino]hexanoate

To a solution of 2-[3-[[5-[[(1S)-1-methoxycarbonyl-4,4-dimethyl-pentyl]carbamoyl]-2-pyridyl]oxy]phenoxy]acetic acid (600 mg, 1.07 mmol, 1 eq, TFA) and 2-(2-prop-2-ynoxyethoxy) ethanamine (123.06 mg, 859.45 μmol, 0.8 eq) in DMF (6 mL) was added HATU (816.97 mg, 2.15 mmol, 2 eq) and DIEA (694.23 mg, 5.37 mmol, 935.62 μL, 5 eq) at 0° C. The mixture was stirred at 20° C. for 1 hr. The reaction mixture was quenched by addition H₂O 5 mL, and extracted with EtOAc 15 mL (5 mL*3). The combined organic layers were washed with brine 20 mL (10 mL*2), dried over Na₂SO₄, filtered and concentrated under reduced pressure. The residue was purified by flash silica gel chromatography (ISCO®; 40 g SepaFlash® Silica Flash Column, Eluent of 0˜87% Ethyl acetate/Petroleum ether gradient at 100 mL/min) to give compound methyl (2S)-5,5-dimethyl-2-[[6-[3-[2-oxo-2-[2-(2-prop-2-ynoxyethoxy)ethylamino]ethoxy]phenoxy]pyridine-3-carbonyl]amino]hexanoate (529 mg, 928.65 μmol, 86.44% yield) as a light brown oil.

### Data:

LCMS (ESI+): m/z 570.3 (M+H)

### 2. General Procedure for Preparation of methyl (2S)-2-[[6-[3-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethylamino]-2-oxo-ethoxy]phenoxy]pyridine-3-carbonyl]amino]-5,5-dimethyl-hexanoate

To a solution of methyl (2S)-5,5-dimethyl-2-[[6-[3-[2-oxo-2-[2-(2-prop-2-ynoxyethoxy)ethylamino]ethoxy]phenoxy]pyridine-3-carbonyl]amino]hexanoate (100 mg, 175.55 μmol, 1 eq) and 3-[(1R)-1-[[6-[2-(5-azidopentoxy)pyrimidin-5-yl]-3-chloro-7-fluoro-2-methyl-1,5-naphthyridin-4-yl]amino]ethyl]-4-fluoro-benzonitrile (99.01 mg, 175.55 μmol, 1 eq) in t-BuOH (1 mL) and H₂O (1 mL) was added copper; sulfate (14.01 mg, 87.77 μmol, 13.47 μL, 0.5 eq) and sodium; (2R)-2-[(1S)-1,2-dihydroxyethyl]-4-hydroxy-5-oxo-2H-furan-3-olate (34.78 mg, 175.55 μmol, 1 eq), then the reaction was stirred for 1 hr at 50° C. The reaction mixture was quenched by addition H₂O 2 mL, and extracted with DCM 16 mL (4 mL*4). The combined organic layers were dried over Na₂SO₄, filtered and concentrated under reduced pressure to give compound methyl (2S)-2-[[6-[3-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethylamino]-2-oxo-ethoxy]phenoxy]pyridine-3-carbonyl]amino]-5,5-dimethyl-hexanoate (250 mg, crude) as a yellow oil.

### Data:

LCMS (ESI+): m/z 1133.1 (M+H)

### 3. General Procedure for Preparation of (2S)-2-[[6-[3-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethylamino]-2-oxo-ethoxy]phenoxy]pyridine-3-carbonyl]amino]-5,5-dimethyl-hexanoic acid

To a solution of methyl (2S)-2-[[6-[3-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethylamino]-2-oxo-ethoxy]phenoxy]pyridine-3-carbonyl]amino]-5,5-dimethyl-hexanoate (250 mg, 220.53 μmol, 1 eq) in THF (2 mL) and H₂O (2 mL) was added LiOH·H₂O (18.51 mg, 441.06 μmol, 2 eq). The mixture was stirred at 20° C. for 20 min. THF was removed. The aqueous layer was adjusted pH to 5 with FA. The residue was purified by prep-HPLC (neutral condition; column: Waters Xbridge Prep OBD C18 150*40 mm*10 um; mobile phase: [H₂O (10 mM NH₄HCO₃)-ACN]; gradient: 30%-65% B over 8.0 min) to give compound (2S)-2-[[6-[3-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethylamino]-2-oxo-ethoxy]phenoxy]pyridine-3-carbonyl]amino]-5,5-dimethyl-hexanoic acid (26.7 mg, 23.85 μmol, 10.81% yield) as a white solid.

The following compounds were prepared according to the general procedure of BF083. The starting materials are either commercially available or may be prepared from commercially available reagents using conventional reactions well known in the art.

### BF083

### (2S)-2-[[6-[3-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethylamino]-2-oxo-ethoxy]phenoxy]pyridine-3-carbonyl]amino]-5,5-dimethyl-hexanoic acid

LCMS (ESI+): m/z 1119.2 (M+H)

¹H NMR (400 MHZ, METHANOL-d₄) δ=8.99 (br s, 1H), 8.61 (br s, 1H), 8.23 (dd, J=2.1, 8.6 Hz, 1H), 7.99 (br s, 1H), 7.91 (br d, J=11.1 Hz, 1H), 7.78 (dd, J=1.9, 6.9 Hz, 1H), 7.61 (ddd, J=2.1, 4.7, 8.5 Hz, 1H), 7.32 (t, J=8.2 Hz, 1H), 7.17 (dd, J=8.6, 10.2 Hz, 1H), 6.97 (d, J=8.7 Hz, 1H), 6.84 (dd, J=2.2, 8.2 Hz, 1H), 6.81-6.73 (m, 2H), 6.44 (q, J=6.8 Hz, 1H), 4.61 (s, 2H), 4.50 (s, 3H), 4.49-4.41 (m, 4H), 3.65-3.58 (m, 4H), 3.57-3.52 (m, 2H), 3.47-3.41 (m, 2H), 2.70 (s, 3H), 2.03-1.94 (m, 3H), 1.94-1.83 (m, 3H), 1.82-1.75 (m, 1H), 1.72 (d, J=6.9 Hz, 3H), 1.51 (quin, J=7.7 Hz, 2H), 1.36-1.28 (m, 2H), 0.89 (s, 9H)

### BF084

### (2S)-2-[[6-[3-[2-[2-[2-[2-[2-[[1-[5-[5-[7-chloro-8-[[(1R)-1-(5-cyano-2-fluoro- phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethylamino]-2-oxo-ethoxy]phenoxy]pyridine-3-carbonyl]amino]-5,5-dimethyl-hexanoic acid

LC/MS [M+1]: 1207.5

¹H NMR (400 MHZ, METHANOL-d₄)·=9.13-8.91 (m, 1H), 8.63 (br d, J=8.3 Hz, 1H), 8.23 (br d, J=7.3 Hz, 1H), 8.18-8.03 (m, 1H), 8.02-7.85 (m, 1H), 7.78 (dd, J=1.6, 6.9 Hz, 1H), 7.65-7.56 (m, 1H), 7.33 (t, J=8.2 Hz, 1H), 7.18 (dd, J=8.8, 10.0 Hz, 1H), 6.98 (br d, J=7.9 Hz, 1H), 6.86 (dd, J=2.1, 8.3 Hz, 1H), 6.83-6.73 (m, 2H), 6.43 (q, J=6.6 Hz, 1H), 4.61 (br s, 2H), 4.54-4.38 (m, 7H), 3.68-3.51 (m, 15H), 3.46-3.41 (m, 2H), 2.69 (br s, 3H), 2.02 (br s, 3H), 1.96-1.83 (m, 3H), 1.71 (d, J=6.8 Hz, 3H), 1.53 (br d, J=5.4 Hz, 2H), 1.31 (br d, J=1.7 Hz, 2H), 0.89 (s, 9H)

### BF086

### (2S)-2-[[6-[3-[2-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethoxy]ethoxy]ethylamino]-2-oxo-ethoxy]phenoxy]pyridine-3-carbonyl]amino]-5,5-dimethyl-hexanoic acid

LC/MS [M+1]: 1295.5

¹H NMR (400 MHZ, METHANOL-d₄) δ=8.98 (s, 2H), 8.62 (d, J=2.2 Hz, 1H), 8.24 (dd, J=2.5, 8.6 Hz, 1H), 8.00 (s, 1H), 7.90 (d, J=11.6 Hz, 1H), 7.78 (dd, J=2.0, 6.9 Hz, 1H), 7.61 (ddd, J=2.1, 4.8, 8.4 Hz, 1H), 7.34 (t, J=8.3 Hz, 1H), 7.18 (dd, J=8.7, 10.1 Hz, 1H), 6.99 (d, J=8.7 Hz, 1H), 6.87 (dd, J=1.9, 8.4 Hz, 1H), 6.81 (t, J=2.2 Hz, 1H), 6.77 (dd, J=1.5, 8.1 Hz, 1H), 6.44 (q, J=6.7 Hz, 1H), 4.62 (s, 2H), 4.54-4.43 (m, 7H), 3.66-3.51 (m, 22H), 3.47-3.42 (m, 2H), 2.70 (s, 3H), 2.08-1.85 (m, 5H), 1.85-1.75 (m, 1H), 1.72 (d, J=6.8 Hz, 3H), 1.58-1.48 (m, 2H), 1.40-1.26 (m, 2H), 0.90 (s, 9H)

### BF085

### 1. General Procedure for Preparation of tert-butyl N-[2-[2-[2-[2-[2-[2-[2-(5-bromopyrimidin-2-yl)oxyethoxy]ethoxy]ethoxy]ethoxy]ethoxy]ethoxy]ethyl]carbamate

To a solution of tert-butyl N-[2-[2-[2-[2-[2-[2-(2-hydroxyethoxy)ethoxy]ethoxy]ethoxy]ethoxy]ethoxy]ethyl]carbamate (1 g, 2.35 mmol, 1 eq) in DMF (10 mL) was added NaH (187.99 mg, 4.70 mmol, 60% purity, 2 eq) at 0° C. The mixture was stirred at 0° C. for 0.5 hr. To the mixture was added 5-bromo-2-chloro-pyrimidine (500.04 mg, 2.59 mmol, 1.1 eq) and stirred at 20° C. for another 0.5 hr. The reaction mixture was quenched by addition NH₄Cl 10 mL at 0° C., and extracted with EtOAc 30 mL (10 mL*3). The combined organic layers were washed with brine 60 mL (30 mL*2), dried over Na₂SO₄, filtered and concentrated under reduced pressure. The crude product was purified by reversed-phase HPLC (column: C18 20-35 um 100 A 330 g; mobile phase: [water-ACN]; B %: 0%-60% @ 100 mL/min) to give compound tert-butyl N-[2-[2-[2-[2-[2-[2-[2-(5-bromopyrimidin-2-yl)oxyethoxy]ethoxy]ethoxy]ethoxy]ethoxy]ethoxy]ethyl]carbamate (800 mg, 1.37 mmol, 58.44% yield) as a brown oil.

### Data:

LCMS (ESI⁺): m/z 584.3 (M+H)

### 2. General Procedure for Preparation of [2-[2-[2-[2-[2-[2-[2-[2-(tert-butoxycarbonylamino)ethoxy]ethoxy]ethoxy]ethoxy]ethoxy]ethoxy]ethoxy]pyrimidin-5-yl]boronic acid

A mixture of tert-butyl N-[2-[2-[2-[2-[2-[2-[2-(5-bromopyrimidin-2-yl)oxyethoxy]ethoxy]ethoxy]ethoxy]ethoxy]ethoxy]ethyl]carbamate (595 mg, 1.02 mmol, 1 eq), 4,4,5,5-tetramethyl-2-(4,4,5,5-tetramethyl-1,3,2-dioxaborolan-2-yl)-1,3,2-dioxaborolane (389.09 mg, 1.53 mmol, 1.5 eq), KOAc (300.75 mg, 3.06 mmol, 3 eq) and Pd(dppf)Cl₂ (74.74 mg, 102.15 μmol, 0.1 eq) in dioxane (6 mL) was degassed and purged with N₂ for 3 times, and then the mixture was stirred at 95° C. for 1 hr under N₂ atmosphere. The reaction mixture of [2-[2-[2-[2-[2-[2-[2-[2-(tertbutoxycarbonylamino) ethoxy]ethoxy]ethoxy]ethoxy]ethoxy]ethoxy]ethoxy]pyrimidin-5-yl]boronic acid (560 mg, crude) in dioxane (6 mL) was obtained as a black liquid, which was used to next step without purification.

### Data:

LCMS (ESI⁺): m/z 548.0 (M+H)

### 3. General Procedure for Preparation of tert-butyl N-[2-[2-[2-[2-[2-[2-[2-[5-(7,8-dichloro-3-fluoro-6-methyl-1,5-naphthyridin-2-yl)pyrimidin-2-yl]oxyethoxy]ethoxy]ethoxy]ethoxy]ethoxy]ethoxy]ethyl]carbamate

A mixture of [2-[2-[2-[2-[2-[2-[2-[2-(tertbutoxycarbonylamino)ethoxy]ethoxy]ethoxy]ethoxy]ethoxy]ethoxy]ethoxy]pyrimidin-5-yl]boronic acid (280 mg, 511.51 μmol, 1 eq), 6-bromo-3,4-dichloro-7-fluoro-2-methyl-1,5-naphthyridine (174.40 mg, 562.66 μmol, 1.1 eq), Cs₂CO₃ (499.98 mg, 1.53 mmol, 3 eq) and Pd(PPh₃)₂Cl₂ (35.90 mg, 51.15 μmol, 0.1 eq) in dioxane (3 mL) and H₂O (0.3 mL) was degassed and purged with N₂ for 3 times, and then the mixture was stirred at 85° C. for 1 hr under N₂ atmosphere. The reaction mixture was quenched by addition H₂O 5 mL, and extracted with DCM 15 mL (5 mL*3). The combined organic layers were dried over Na₂SO₄, filtered and concentrated under reduced pressure. The residue was purified by prep-TLC (SiO₂, Ethyl acetate:Methanol=10:1) to give compound tert-butyl N-[2-[2-[2-[2-[2-[2-[2-[5-(7,8-dichloro-3-fluoro-6-methyl-1,5-naphthyridin-2-yl)pyrimidin-2-yl]oxyethoxy]ethoxy]ethoxy]ethoxy]ethoxy]ethoxy]ethyl]carbamate (274 mg, 374.00 μmol, 73.12% yield) as a yellow oil.

### Data:

LCMS (ESI⁺): m/z 732.8 (M+H)

### 4 General Procedure for Preparation of tert-butyl N-[2-[2-[2-[2-[2-[2-[2-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxyethoxy]ethoxy]ethoxy]ethoxy]ethoxy]ethoxy]ethyl]carbamate

A mixture of tert-butyl N-[2-[2-[2-[2-[2-[2-[2-[5-(7,8-dichloro-3-fluoro-6-methyl-1,5-naphthyridin-2-yl)pyrimidin-2-yl]oxyethoxy]ethoxy]ethoxy]ethoxy]ethoxy]ethoxy]ethyl]carbamate (210 mg, 286.64 μmol, 1 eq), 3-[(1R)-1-aminoethyl]-4-fluoro-benzonitrile (47.06 mg, 286.64 μmol, 1 eq), Pd₂(dba)₃ (26.25 mg, 28.66 μmol, 0.1 eq), BINAP (35.70 mg, 57.33 μmol, 0.2 eq) and Cs₂CO₃ (186.79 mg, 573.28 μmol, 2 eq) in dioxane (2 mL) was degassed and purged with N₂ for 3 times, and then the mixture was stirred at 100° C. for 12 hr under N₂ atmosphere. The reaction mixture was quenched by addition H₂O 2 mL, and extracted with EtOAc 9 mL (3 mL*3). The combined organic layers were dried over Na₂SO₄, filtered and concentrated under reduced pressure. The residue was purified by prep-TLC (SiO₂, Ethyl acetate:Methanol=10:1) to give compound tert-butyl N-[2-[2-[2-[2-[2-[2-[2-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxyethoxy]ethoxy]ethoxy]ethoxy]ethoxy]ethoxy]ethyl]carbamate (148 mg, 172.02 μmol, 60.01% yield) as a yellow oil.

### Data:

LCMS (ESI⁺): m/z 860.3 (M+H)

### 5 General Procedure for Preparation of 3-[(1R)-1-[[6-[2-[2-[2-[2-[2-[2-[2-(2-aminoethoxy)ethoxy]ethoxy]ethoxy]ethoxy]ethoxy]ethoxy]pyrimidin-5-yl]-3-chloro-7-fluoro-2-methyl-1,5-naphthyridin-4-yl]amino]ethyl]-4-fluoro-benzonitrile

A mixture of tert-butyl N-[2-[2-[2-[2-[2-[2-[2-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxyethoxy]ethoxy]ethoxy]ethoxy]ethoxy]ethoxy]ethyl]carbamate (148 mg, 172.02 μmol, 1 eq) in DCM (1.5 mL) and TFA (0.3 mL) was stirred at 15° C. for 20 min. The reaction mixture was concentrated under reduced pressure to give compound 3-[(1R)-1-[[6-[2-[2-[2-[2-[2-[2-[2-(2-aminoethoxy)ethoxy]ethoxy]ethoxy]ethoxy]ethoxy]ethoxy]pyrimidin-5-yl]-3-chloro-7-fluoro-2-methyl-1,5-naphthyridin-4-yl]amino]ethyl]-4-fluoro-benzonitrile (260 mg, crude, TFA) as a yellow oil.

### Data:

LCMS (ESI⁺): m/z 760.4 (M+H)

### 6 General Procedure for Preparation of methyl (2S)-2-[[6-[3-[2-[2-[2-[2-[2-[2-[2-[2-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxyethoxy]ethoxy]ethoxy]ethoxy]ethoxy]ethoxy]ethylamino]-2-oxo-ethoxy]phenoxy]pyridine-3-carbonyl]amino]-5,5-dimethyl-hexanoate

To a solution of 3-[(1R)-1-[[6-[2-[2-[2-[2-[2-[2-[2-(2-aminoethoxy)ethoxy]ethoxy]ethoxy]ethoxy]ethoxy]ethoxy]pyrimidin-5-yl]-3-chloro-7-fluoro-2-methyl-1,5-naphthyridin-4-yl]amino]ethyl]-4-fluoro-benzonitrile (240 mg, 274.52 μmol, 1 eq, TFA) and 2-[3-[[5-[[(1S)-1-methoxycarbonyl-4,4-dimethyl-pentyl]carbamoyl]-2-pyridyl]oxy]phenoxy]acetic acid (137.99 mg, 247.07 μmol, 0.9 eq, TFA) in DMF (2.5 mL) was added HATU (208.76 mg, 549.04 μmol, 2 eq) and DIEA (141.92 mg, 1.10 mmol, 191.27 μL, 4 eq) at 0° C. The mixture was stirred at 15° C. for 1 hr. The reaction mixture was quenched by addition H₂O 3 mL, and extracted with DCM 15 mL (5 mL*3). The combined organic layers were washed with brine 40 mL (20 mL*2), dried over Na₂SO₄, filtered and concentrated under reduced pressure. The crude product was purified by reversed-phase HPLC (column: C18 20-35 μm 100 A 40 g; mobile phase: [water-ACN]; B %: 0%-80% @ 80 mL/min) to give compound methyl (2S)-2-[[6-[3-[2-[2-[2-[2-[2-[2-[2-[2-[5-[7-chloro-8-[[(1R)-1-(5-cyano-2-fluorophenyl)ethyl]amino]-3-fluoro-6-methyl-1,5-naphthyridin-2-yl]pyrimidin-2-yl]oxyethoxy]ethoxy]ethoxy]ethoxy]ethoxy]ethoxy]ethylamino]-2-oxo-ethoxy]phenoxy]pyridine-3-carbonyl]amino]-5,5-dimethyl-hexanoate (131 mg, 110.39 μmol, 40.21% yield) as a yellow oil.

### Data:

LCMS (ESI⁺): m/z 1186.1 (M+H)

### 7 General Procedure for Preparation of (2S)-2-[[6-[3-[2-[2-[2-[2-[2-[2-[2-[2-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxyethoxy]ethoxy]ethoxy]ethoxy]ethoxy]ethoxy]ethylamino]-2-oxo-ethoxy]phenoxy]pyridine-3-carbonyl]amino]-5,5-dimethyl-hexanoic acid

The deprotection of methyl (2S)-2-[[6-[3-[2-[2-[2-[2-[2-[2-[2-[2-[5-[7-chloro-8-[[(1R)-1-(5-cyano-2-fluorophenyl)ethyl]amino]-3-fluoro-6-methyl-1,5-naphthyridin-2-yl]pyrimidin-2-yl]oxyethoxy]ethoxy]ethoxy]ethoxy]ethoxy]ethoxy]ethylamino]-2-oxo-ethoxy]phenoxy]pyridine-3-carbonyl]amino]-5,5-dimethyl-hexanoate (131 mg, 110.39 μmol, 1 eq) in THF (1 mL) and H₂O (1 mL) was performed analogously to described above for BF083 to give the compound of the title (BF085) (56 mg, 47.62 μmol, 43.14% yield, 99.716% purity) as a white solid.

### Data:

LCMS (ESI⁺): m/z 1172.4 (M+H)

¹H NMR (400 MHZ, METHANOL-d₄) δ=8.98 (s, 1H), 8.61 (d, J=2.2 Hz, 1H), 8.23 (dd, J=2.5, 8.6 Hz, 1H), 7.91 (d, J=11.5 Hz, 1H), 7.79 (dd, J=2.1, 7.0 Hz, 1H), 7.61 (ddd, J=2.2, 4.8, 8.4 Hz, 1H), 7.34 (t, J=8.2 Hz, 1H), 7.19 (dd, J=8.6, 10.1 Hz, 1H), 6.99 (d, J=8.7 Hz, 1H), 6.87 (dd, J=2.1, 8.1 Hz, 1H), 6.81 (t, J=2.3 Hz, 1H), 6.77 (dd, J=1.5, 8.0 Hz, 1H), 6.44 (q, J=6.8 Hz, 1H), 4.65 (dd, J=3.9, 5.4 Hz, 2H), 4.52 (s, 2H), 4.51-4.46 (m, 1H), 3.94-3.89 (m, 2H), 3.74-3.69 (m, 2H), 3.68-3.63 (m, 2H), 3.63-3.53 (m, 19H), 3.45 (q, J=4.8 Hz, 2H), 2.71 (s, 3H), 2.02-1.90 (m, 1H), 1.87-1.75 (m, 1H), 1.72 (d, J=6.8 Hz, 3H), 1.38-1.28 (m, 2H), 0.91 (s, 9H)

### BF089, BF088

### 1. General Procedure for Preparation of tert-butyl 3-(cyanomethoxy)azetidine-1-carboxylate

To a solution of tert-butyl 3-hydroxyazetidine-1-carboxylate (100 mg, 577.34 μmol, 1 eq) in THF (2 mL) was added NaH (34.64 mg, 866.01 μmol, 60% purity, 1.5 eq) at 0° C., then the reaction was stirred for 30 min, then 2-chloroacetonitrile (43.59 mg, 577.34 μmol, 36.54 μL, 1 eq) and NaI (8.65 mg, 57.73 μmol, 0.1 eq) was added and the mixture was stirred at 0° C. for 2 h under N₂ atmosphere. The mixture was added slowly into saturated NH₄Cl solution 3 mL at 0° C. under N₂, then extracted with EtOAc (3*3 mL). The organic layer was dried over Na₂SO₄, filtered and filtrate was concentrated to give a residue which was purified by prep-TLC (SiO₂, petroleum ether:ethyl acetate=1:1) to give tert-butyl 3-(cyanomethoxy)azetidine-1-carboxylate (10 mg, 47.12 μmol, 8.16% yield) as a yellow oil.

### Data:

¹H NMR (400 MHZ, CHLOROFORM-d) δ=4.42 (tt, J=4.1, 6.4 Hz, 1H), 4.24 (s, 2H), 4.19-4.13 (m, 2H), 3.92 (dd, J=4.1, 10.4 Hz, 2H), 1.45 (s, 9H)

### 2. General Procedure for Preparation of tert-butyl 3-(2-aminoethoxy)azetidine-1-carboxylate

To a solution of tert-butyl 3-(cyanomethoxy)azetidine-1-carboxylate (2 g, 9.42 mmol, 1 eq) in EtOH (150 mL) and NH₃·H₂O (3.30 g, 28.27 mmol, 3.63 mL, 30% purity, 3 eq) was added Raney-Ni (434.78 mg, 5.07 mmol, 5.39e-1 eq) under N₂ atmosphere. The suspension was degassed and purged with H₂ for 3 times. The mixture was stirred under H₂ (50 Psi) at 30° C. for 12 h. The reaction mixture was filtered and filtrate was concentrated to give a residue. The residue was purified by column chromatography (SiO₂, EtOAc:MeOH=1/0 to 10/1) to give tert-butyl 3-(2-aminoethoxy)azetidine-1-carboxylate (1.2 g, 5.55 mmol, 58.88% yield) as colorless oil.

### 3. General Procedure for Preparation of tert-butyl 3-[2-[[2-[3-[[5-[[(1S)-1-methoxycarbonyl-4,4-dimethyl-pentyl]carbamoyl]-2-pyridyl]oxy]phenoxy]acetyl]amino]ethoxy]azetidine-1-carboxylate

To a solution of 2-[3-[[5-[[(1S)-1-methoxycarbonyl-4,4-dimethyl-pentyl]carbamoyl]-2-pyridyl]oxy]phenoxy]acetic acid (500 mg, 895.26 μmol, 1 eq, TFA) and tert-butyl 3-(2-aminoethoxy)azetidine-1-carboxylate (154.90 mg, 716.20 μmol, 0.8 eq) in DMF (5 mL) was added HATU (680.81 mg, 1.79 mmol, 2 eq) and DIEA (462.82 mg, 3.58 mmol, 623.75 μL, 4 eq), then the reaction was stirred at 0° C. for 1 h. The reaction mixture was diluted with water 5 mL, and then extracted with EtOAc 15 mL (5 mL*3). The combined organic layers were washed with brine 10 mL, dried over Na₂SO₄, filtered and concentrated under reduced pressure to give a residue which was purified by flash silica gel chromatography (ISCO®; 20 g SepaFlash® Silica Flash Column, Eluent of 50˜100% Ethyl acetate/Petroleum ether gradient @ 100 mL/min) to give tert-butyl 3-[2-[[2-[3-[[5-[[(1S)-1-methoxycarbonyl-4,4-dimethyl-pentyl]carbamoyl]-2-pyridyl]oxy]phenoxy]acetyl]amino]ethoxy]azetidine-1-carboxylate (482 mg, 749.92 μmol, 83.77% yield) as brown oil.

### Data: LCMS (ESI⁺): m/z 643.1 (M+H)

### 4. General Procedure for Preparation of methyl (2S)-2-[[6-[3-[2-[2-(azetidin-3-yloxy)ethylamino]-2-oxo-ethoxy]phenoxy]pyridine-3-carbonyl]amino]-5,5-dimethyl-hexanoate

To a solution of tert-butyl 3-[2-[[2-[3-[[5-[[(1S)-1-methoxycarbonyl-4,4-dimethyl-pentyl]carbamoyl]-2-pyridyl]oxy]phenoxy]acetyl]amino]ethoxy]azetidine-1-carboxylate (482 mg, 749.92 μmol, 1 eq) in DCM (5 mL) was added TFA (1.54 g, 13.46 mmol, 1 mL, 17.95 eq), the reaction was stirred at 20° C. for 1 h. The reaction mixture was concentrated by N₂ to give methyl (2S)-2-[[6-[3-[2-[2-(azetidin-3-yloxy)ethylamino]-2-oxo-ethoxy]phenoxy]pyridine-3-carbonyl]amino]-5,5-dimethyl-hexanoate (490 mg, crude, TFA) as yellow oil.

### Data: LCMS (ESI⁺): m/z 543.3 (M+H)

### 5. General Procedure for Preparation of methyl (2S)-5,5-dimethyl-2-[[6-[3-[2-oxo-2-[2-[1-(2-prop-2-ynoxyacetyl)azetidin-3-yl]oxyethylamino]ethoxy]phenoxy]pyridine-3-carbonyl]amino]hexanoate

To a solution of methyl (2S)-2-[[6-[3-[2-[2-(azetidin-3-yloxy)ethylamino]-2-oxo-ethoxy]phenoxy]pyridine-3-carbonyl]amino]-5,5-dimethyl-hexanoate (490 mg, 746.22 μmol, 1 eq, TFA) and 2-prop-2-ynoxyacetic acid (85.14 mg, 746.22 μmol, 1 eq) in DMF (5 mL) was added DIEA (482.22 mg, 3.73 mmol, 649.89 UL, 5 eq) and HATU (567.47 mg, 1.49 mmol, 2 eq), then the reaction was stirred at 20° C. for 1 h. The reaction mixture was diluted with water 5 mL, and then extracted with EtOAc 25 mL (5 mL*5). The combined organic layers were washed with brine 40 mL (20 mL*2), dried over Na₂SO₄, filtered and concentrated under reduced pressure to give a residue. The residue was purified by flash silica gel chromatography (ISCO®; 20 g SepaFlash® Silica Flash Column, Eluent of 5˜10% Methanol/Ethyl acetate gradient @ 100 mL/min) to give methyl (2S)-5,5-dimethyl-2-[[6-[3-[2-oxo-2-[2-[1-(2-prop-2-ynoxyacetyl)azetidin-3-yl]oxyethylamino]ethoxy]phenoxy]pyridine-3-carbonyl]amino]hexanoate (250 mg, 391.42 μmol, 52.45% yield) as a light yellow oil.

Data: LCMS (ESI⁺): m/z 639.4 (M+H)

¹H NMR (400 MHZ, METHANOL-d₄) δ=8.63 (d, J=2.3 Hz, 1H), 8.25 (dd, J=2.5, 8.6 Hz, 1H), 7.38 (t, J=8.2 Hz, 1H), 7.04 (d, J=8.7 Hz, 1H), 6.91 (dd, J=1.9, 8.3 Hz, 1H), 6.86-6.79 (m, 2H), 4.57-4.50 (m, 3H), 4.46-4.39 (m, 1H), 4.36-4.30 (m, 1H), 4.20 (d, J=2.4 Hz, 2H), 4.12-4.07 (m, 4H), 3.80 (dd, J=3.9, 10.6 Hz, 1H), 3.75 (s, 3H), 3.53-3.45 (m, 4H), 2.92 (t, J=2.4 Hz, 1H), 1.98-1.88 (m, 1H), 1.86-1.75 (m, 1H), 1.33 (ddd, J=4.9, 12.4, 18.9 Hz, 2H), 0.92 (s, 9H).

### 6. General Procedure for Preparation of tert-butyl 3-azidoazetidine-1-carboxylate

To a solution of tert-butyl 3-bromoazetidine-1-carboxylate (10 g, 42.35 mmol, 1 eq) in DMF (130 mL) was added NaN₃ (3.30 g, 50.82 mmol, 1.2 eq), then the reaction was stirred for 1 hr at 20° C. and 11 hr at 80° C. The reaction mixture was quenched by addition H₂O 100 mL and Na₂CO₃ 30 mL, then the reaction was extracted with EtOAc 300 mL (100 mL*3). The combined organic layers were washed with brine 400 mL (200 mL*2), dried over Na₂SO₄, filtered and concentrated under reduced pressure to give a residue. The residue was purified by column chromatography (SiO₂, Petroleum ether/Ethyl acetate=10/1 to 5/1) to give tert-butyl 3-azidoazetidine-1-carboxylate (8.4 g, crude) as a light yellow oil.

Data: ¹H NMR (400 MHZ, CHLOROFORM-d) õ=4.23-4.14 (m, 3H), 3.93-3.85 (m, 2H), 1.44 (s, 9H)

### 7. General Procedure for Preparation of 3-azidoazetidine

A solution of tert-butyl 3-azidoazetidine-1-carboxylate (7.9 g, 39.85 mmol, 1 eq) in DCM (80 mL) and TFA (24.56 g, 215.40 mmol, 16 mL, 5.40 eq) was stirred for 12 hr at 20° C. The mixture was concentrated by N₂ to give 3-azidoazetidine (8 g, crude, TFA) as colorless oil, which was used to next step without purification.

Data: ¹H NMR (400 MHZ, CHLOROFORM-d) δ=4.69 (quin, J=7.1 Hz, 1H), 4.39 (br d, J=2.7 Hz, 2H), 4.13 (br s, 2H)

### 8. General Procedure for Preparation of 2-(3-azidoazetidin-1-yl)ethoxy-tert-butyl-dimethyl-silane

TEA (1.19 g, 11.79 mmol, 1.64 mL, 1 eq) was added to a solution of 3-azidoazetidine (2.5 g, 11.79 mmol, 1 eq, TFA) in MeOH (30 mL) until pH=8˜9, then 2-[tert-butyl(dimethyl)silyl]oxyacetaldehyde (4.11 g, 23.57 mmol, 4.49 mL, 2 eq), AcOH (707.73 mg, 11.79 mmol, 674.67 μL, 1 eq) and NaBH₃CN (740.61 mg, 11.79 mmol, 1 eq) was added to above mixture. The reaction was stirred at 20° C. for 12 h. The reaction was concentrated to give a residue which was purified by reversed-phase HPLC (column: 80 g Agela C18; mobile phase: [water-ACN]; B %: 60-90% 30 min; 90% 10 min @ 100 mL/min) to give 2-(3-azidoazetidin-1-yl)ethoxy-tert-butyl-dimethyl-silane (2.34 g, 9.13 mmol, 77.43% yield) as colorless oil

### Data:

LCMS (ESI⁺): m/z 257.0 (M+H)

¹H NMR (400 MHZ, CHLOROFORM-d) δ=4.26 (br t, J=6.6 Hz, 1H), 3.96 (br t, J=7.6 Hz, 2H), 3.72 (t, J=5.4 Hz, 2H), 3.33 (br t, J=7.9 Hz, 2H), 2.78 (t, J=5.4 Hz, 2H), 0.90 (s, 9H), 0.07 (s, 6H)

### 9. General Procedure for Preparation of 2-(3-azidoazetidin-1-yl) ethanol

To a solution of 2-(3-azidoazetidin-1-yl)ethoxy-tert-butyl-dimethyl-silane (2.2 g, 8.58 mmol, 1 eq) in THF (30 mL) was added TBAF (1 M, 17.16 mL, 2 eq), then the reaction was stirred at 20° C. for 2 h. The reaction was concentrated to give a residue which was purified by column chromatography (SiO₂, Petroleum ether/Ethyl acetate (0.1% NH₃·H₂O)=1/1 to 0/1) to give 2-(3-azidoazetidin-1-yl) ethanol (1.18 g, 8.30 mmol, 96.75% yield) as yellow oil.

### Data:

LCMS (ESI⁺): m/z 142.9 (M+H)

¹H NMR (400 MHZ, CHLOROFORM-d) õ=4.10-4.01 (m, 1H), 3.67 (dd, J=6.8, 8.3 Hz, 2H), 3.59-3.50 (m, 2H), 3.11 (dd, J=6.4, 8.2 Hz, 2H), 2.68-2.60 (m, 2H), 2.32 (br s, 1H)

### 10. General Procedure for Preparation of 2-[2-(3-azidoazetidin-1-yl)ethoxy]-5-bromo-pyrimidine

To a solution of 2-(3-azidoazetidin-1-yl) ethanol (1.1 g, 7.74 mmol, 1 eq) in THF (15 mL) was added NaH (371.38 mg, 9.29 mmol, 60% purity, 1.2 eq) at 0° C. under N₂, the reaction was stirred at 0° C. for 0.5 h, 5-bromo-2-chloro-pyrimidine (1.50 g, 7.74 mmol, 1 eq) was added to above mixture and the reaction was stirred at 20° C. for 2 h. The reaction was quenched by addition ice water 10 mL and extracted with EtOAc 30 mL (10 mL*3). The combined organic layers were washed with brine 20 mL (10 mL*2), dried over Na₂SO₄, filtered and concentrated under reduced pressure to give a residue. The residue was purified by flash silica gel chromatography (ISCO®; 40 g SepaFlash® Silica Flash Column, Eluent of 50˜100% Ethyl acetate/Petroleum ether gradient @ 100 mL/min) to give 2-[2-(3-azidoazetidin-1-yl)ethoxy]-5-bromo-pyrimidine (2.09 g, 6.99 mmol, 90.30% yield) as yellow oil.

### Data:

LCMS (ESI⁺): m/z 299.0 (M+H)

¹H NMR (400 MHZ, CHLOROFORM-d) δ=8.52 (s, 2H), 4.35 (t, J=5.6 Hz, 2H), 4.04 (quin, J=6.3 Hz, 1H), 3.79-3.66 (m, 2H), 3.22-3.10 (m, 2H), 2.87 (t, J=5.6 Hz, 2H)

### 11. General Procedure for Preparation of [2-[2-(3-azidoazetidin-1-yl)ethoxy]pyrimidin-5-yl]boronic acid

A mixture of 2-[2-(3-azidoazetidin-1-yl)ethoxy]-5-bromo-pyrimidine (120 mg, 401.17 μmol, 1 eq), 4,4,5,5-tetramethyl-2-(4,4,5,5-tetramethyl-1,3,2-dioxaborolan-2-yl)-1,3,2-dioxaborolane (203.74 mg, 802.34 μmol, 2 eq), KOAc (78.74 mg, 802.34 μmol, 2.00 eq) and [2-(2-aminophenyl)phenyl]-methylsulfonyloxy-palladium; dichloromethane; tricyclohe ylphosphane (29.49 mg, 40.12 μmol, 0.1 eq) in toluene (2 mL) was degassed and purged with N₂ for 3 times, and then the mixture was stirred at 70° C. for 12 h under N₂ atmosphere. The reaction mixture was filtered and filtrate was concentrated to give a residue. The residue was purified by reversed-phase HPLC (column: C18 20-35 um 100 A 330 g; mobile phase: [water-ACN]; B %: 25%-35% @ 100 mL/min) to give [2-[2-(3-azidoazetidin-1-yl)ethoxy]pyrimidin-5-yl]boronic acid (500 mg, 1.89 mmol, 47.20% yield) as brown solid.

### Data:

LCMS (ESI⁺): m/z 264.9 (M+H)

### 12. General Procedure for Preparation of 6-[2-[2-(3-azidoazetidin-1-yl)ethoxy]pyrimidin-5-yl]-3,4-dichloro-7-fluoro-2-methyl-1,5-naphthyridine

The coupling of 6-bromo-3,4-dichloro-7-fluoro-2-methyl-1,5-naphthyridine was performed in analogous conditions as described in step 3 of compound BF085 to give 6-[2-[2-(3-azidoazetidin-1-yl)ethoxy]pyrimidin-5-yl]-3,4-dichloro-7-fluoro-2-methyl-1,5-naphthyridine (260 mg, 578.72 μmol, 76.40% yield) as a red solid.

### Data:

LCMS (ESI⁺): m/z 448.9 (M+H)

### 13. General Procedure for Preparation of 3-[(1R)-1-[[6-[2-[2-(3-azidoazetidin-1-yl)ethoxy]pyrimidin-5-yl]-3-chloro-7-fluoro-2-methyl-1,5-naphthyridin-4-yl]amino]ethyl]-4-fluoro-benzonitrile

The coupling of 3-[(1R)-1-aminoethyl]-4-fluoro-benzonitrile was done analogously as described in step 4 of the preparation of BF085 above to give 3-[(1R)-1-[[6-[2-[2-(3-azidoazetidin-1-yl)ethoxy]pyrimidin-5-yl]-3-chloro-7-fluoro-2-methyl-1,5-naphthyridin-4-yl]amino]ethyl]-4-fluoro-benzonitrile (60 mg, 103.99 μmol, 23.36% yield) as yellow solid.

### Data:

LCMS (ESI⁺): m/z 577.2 (M+H)

### 14. General Procedure for Preparation of methyl (2S)-2-[[6-[3-[2-[2-[1-[2-[[1-[1-[2-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxyethyl]azetidin-3-yl]triazol-4-yl]methoxy]acetyl]azetidin-3-yl]oxyethylamino]-2-oxo-ethoxy]phenoxy]pyridine-3-carbonyl]amino]-5,5-dimethyl-hexanoate

The click reaction of 3-[(1R)-1-[[6-[2-[2-(3-azidoazetidin-1-yl)ethoxy]pyrimidin-5-yl]-3-chloro-7-fluoro-2-methyl-1,5-naphthyridin-4-yl]amino]ethyl]-4-fluoro-benzonitrile (55 mg, 95.32 μmol, 1 eq) and methyl (2S)-5,5-dimethyl-2-[[6-[3-[2-oxo-2-[2-[1-(2-prop-2-ynoxyacetyl)azetidin-3-yl]oxyethylamino]ethoxy]phenoxy]pyridine-3-carbonyl]amino]hexanoate was performed analogously as step 2 of the preparation of BF083 described above. The residue was purified by reversed-phase HPLC to give methyl (2S)-2-[[6-[3-[2-[2-[1-[2-[[1-[1-[2-[5-[7-chloro-8-[[(1R)-1-(5-cyano-2-fluoro- phenyl)ethyl]amino]-3-fluoro-6-methyl-1,5-naphthyridin-2-yl]pyrimidin-2-yl]oxyethyl]azetidin-3-yl]triazol-4-yl]methoxy]acetyl]azetidin-3-yl]oxyethylamino]-2-oxo-ethoxy]phenoxy]pyridine-3-carbonyl]amino]-5,5-dimethyl-hexanoate (23 mg, 18.92 μmol, 19.85% yield) as white solid.

### Data:

LCMS (ESI⁺): m/z 1216.1 (M+H)

### 15. General Procedure for Preparation of (2S)-2-[[6-[3-[2-[2-[1-[2-[[1-[1-[2-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxyethyl]azetidin-3-yl]triazol-4-yl]methoxy]acetyl]azetidin-3-yl]oxyethylamino]-2-oxo-ethoxy]phenoxy]pyridine-3-carbonyl]amino]-5,5-dimethyl-hexanoic acid

The deprotection of methyl (2S)-2-[[6-[3-[2-[2-[1-[2-[[1-[1-[2-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxyethyl]azetidin-3-yl]triazol-4-yl]methoxy]acetyl]azetidin-3-yl]oxyethylamino]-2-oxo-ethoxy]phenoxy]pyridine-3-carbonyl]amino]-5,5-dimethyl-hexanoate (23 mg, 18.92 μmol, 1 eq) in THF (1 mL) and H₂O (1 mL) was performed analogously to the description above of BF083 to give the compound of the title BF089 as a white solid.

### Data:

LCMS (ESI⁺): m/z 1201.3 (M+H)

¹H NMR (400 MHZ, METHANOL-d₄) δ=9.02 (d, J=1.3 Hz, 2H), 8.61 (d, J=2.3 Hz, 1H), 8.26-8.18 (m, 2H), 7.93 (d, J=11.5 Hz, 1H), 7.78 (dd, J=2.1, 7.0 Hz, 1H), 7.61 (ddd, J=2.3, 4.6, 8.5 Hz, 1H), 7.33 (t, J=8.2 Hz, 1H), 7.18 (dd, J=8.7, 10.3 Hz, 1H), 6.98 (d, J=8.5 Hz, 1H), 6.86 (dd, J=1.9, 8.3 Hz, 1H), 6.82-6.73 (m, 2H), 6.45 (q, J=6.6 Hz, 1H), 5.39 (t, J=6.7 Hz, 1H), 4.67-4.63 (m, 2H), 4.62 (s, 2H), 4.53 (s, 2H), 4.48 (br dd, J=5.3, 7.9 Hz, 1H), 4.42-4.35 (m, 1H), 4.34-4.27 (m, 1H), 4.18-4.10 (m, 3H), 4.06 (s, 2H), 4.02 (br d, J=3.0 Hz, 0.5H), 3.94 (br t, J=7.3 Hz, 2H), 3.77 (dd, J=4.2, 11.1 Hz, 1H), 3.50-3.43 (m, 4H), 3.23 (br t, J=4.9 Hz, 2H), 3.17-3.10 (m, 0.5H), 2.71 (s, 3H), 2.02-1.89 (m, 1H), 1.86-1.76 (m, 1H), 1.72 (d, J=6.9 Hz, 3H), 1.33 (dt, J=5.6, 11.7 Hz, 2H), 0.90 (s, 9H)

The following compounds were prepared according to the general procedure of BF089. The starting materials are either commercially available or may be prepared from commercially available reagents using conventional reactions well known in the art.

### BF088

### (2S)-2-[[6-[3-[2-[2-[1-[2-[[1-[2-[2-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxyethoxy]ethyl]triazol-4-yl]methoxy]acetyl]azetidin-3-yl]oxyethylamino]-2-oxo-ethoxy]phenoxy]pyridine-3-carbonyl]amino]-5,5-dimethyl-hexanoic acid

LC/MS [M+1]: 1190.3

¹H NMR (400 MHZ, METHANOL-d4) δ=9.00 (s, 2H), 8.60 (d, J=2.4 Hz, 1H), 8.22 (dd, J=2.5, 8.6 Hz, 1H), 8.06 (s, 1H), 7.92 (d, J=11.6 Hz, 1H), 7.80 (dd, J=1.9, 6.9 Hz, 1H), 7.61 (ddd, J=2.1, 4.8, 8.4 Hz, 1H), 7.31 (t, J=8.2 Hz, 1H), 7.18 (dd, J=8.6, 10.1 Hz, 1H), 6.97 (d, J=8.6 Hz, 1H), 6.84 (dd, J=2.3, 8.3 Hz, 1H), 6.80-6.72 (m, 2H), 6.43 (d, J=6.8 Hz, 1H), 4.65-4.57 (m, 6H), 4.54-4.46 (m, 3H), 4.39-4.32 (m, 1H), 4.30-4.23 (m, 1H), 4.10 (br dd, J=6.5, 10.8 Hz, 1H), 4.04-3.94 (m, 5H), 3.92-3.86 (m, 2H), 3.73 (br d, J=10.8 Hz, 1H), 3.50-3.39 (m, 4H), 2.70 (s, 3H), 2.01-1.90 (m, 1H), 1.86-1.77 (m, 1H), 1.75-1.69 (m, 3H), 1.41-1.27 (m, 2H), 0.91 (s, 9H)

### BF090

### 1. General Procedure for Preparation of benzyl 4-[2-(tert-butoxycarbonylamino)ethoxy]piperidine-1-carboxylate

To a solution of benzyl 4-hydroxypiperidine-1-carboxylate (5 g, 21.25 mmol, 1 eq) in DMF (100 mL) was added NaH (1.02 g, 25.50 mmol, 60% purity, 1.2 eq) at 0° C., then the reaction was stirred for 1 h at 0° C., tert-butyl 2,2-dioxooxathiazolidine-3-carboxylate (5.69 g, 25.50 mmol, 1.2 eq) was added to above mixture and the reaction was stirred at 25° C. for 11 h. The reaction mixture was quenched with sat. NH₄Cl 50 mL and extracted with ethyl acetate 500 mL (100 mL*5). The combined organic layers were dried over anhydrous Na₂SO₄, filtered and concentrated under reduced pressure to give a residue. The residue was purified by flash silica gel chromatography (ISCO; 40 g SepaFlash Silica Flash Column, Eluent of 0˜15% Ethyl acetate/Petroleum ether gradient @ 120 mL/min, Petroleum ether:Ethyl acetate=1:1) to give compound benzyl 4-[2-(tert-butoxycarbonylamino)ethoxy]piperidine-1-carboxylate (1.4 g, 3.70 mmol, 17.41% yield) as a colorless oil.

### Data:

LCMS (ESI⁺): m/z 379.1 (M+H)

### 2. General Procedure for Preparation of tert-butyl N-[2-(4-piperidyloxy)ethyl]carbamate

To a solution of benzyl 4-[2-(tert-butoxycarbonylamino)ethoxy]piperidine-1-carboxylate (1.4 g, 3.70 mmol, 1 eq) in THF (15 mL) was added Pd/C (1.5 g, 3.70 mmol, 10% purity, 1.00 eq). Then the reaction was degassed and purged with H₂ for 3 times, and then stirred at 25° C. for 12 hr under H₂ atmosphere (15 psi). The catalyst was removed by filtration through celite, which was then washed with MeOH (30 mL). The filtrate was concentrated to give an oil. The crude was filtered and concentrated under the reduced pressure to give compound tert-butyl N-[2-(4-piperidyloxy)ethyl]carbamate (740 mg, 3.03 mmol, 92.50% yield) as a yellow oil.

### 3. General Procedure for Preparation of tert-butyl N-[2-[[1-(2-prop-2-ynoxyacetyl)-4-piperidyl]oxy]ethyl]carbamate

To a solution of tert-butyl N-[2-(4-piperidyloxy)ethyl]carbamate (690 mg, 2.82 mmol, 1 eq) and 2-prop-2-ynoxyacetic acid (322.22 mg, 2.82 mmol, 1 eq) in DMF (10 mL) was added DIEA (1.09 g, 8.47 mmol, 1.48 mL, 3 eq) and CMPI (1.08 g, 4.24 mmol, 1.5 eq). Then, it was stirred at 20° C. for 2 hr. The reaction was added into water (15 mL), then extracted with ethyl acetate (3*10 mL). The combined organic layers were washed with brine (3*10 mL) and dried over anhydrous sodium sulfate. It was filtered and concentrated under reduced pressure to give a residue. The residue was purified by flash chromatography (silica gel, petroleum ether/ethyl acetate=1/0 to 0/1) to give a crude product. The residue was purified by flash chromatography (silica gel, petroleum ether/ethyl acetate=1/0 to 0/1) to give compound tert-butyl N-[2-[[1-(2-prop-2-ynoxyacetyl)-4-piperidyl]oxy]ethyl]carbamate (126 mg, crude) as a yellow oil.

### Data:

¹H NMR (400 MHZ, CHLOROFORM-d) δ ppm 1.38 (s, 9H) 1.54 (m, 2H) 1.77 (m, 2H) 2.40 (m, 1H) 3.24 (m, 4H) 3.47 (m, 3H) 3.58 (m, 1H) 3.79 (m, 1H) 4.19 (s, 2H) 4.22 (d, J=2.20 Hz, 2H) 4.77 (m, 1H)

### 4. General Procedure for Preparation of 1-[4-(2-aminoethoxy)-1-piperidyl]-2-prop-2-ynoxy-ethanone

A mixture of tert-butyl N-[2-[[1-(2-prop-2-ynoxyacetyl)-4-piperidyl]oxy]ethyl]carbamate (116 mg, 340.76 μmol, 1 eq) in DCM (1 mL) and TFA (0.2 mL) was stirred at 20° C. for 1 hr. The crude product was concentrated under the reduced pressure to give compound 1-[4-(2-aminoethoxy)-1-piperidyl]-2-prop-2-ynoxy-ethanone (120 mg, 338.68 μmol, 99.39% yield, TFA) as a yellow oil.

### 5. General Procedure for Preparation of methyl (2S)-5,5-dimethyl-2-[[6-[3-[2-oxo-2-[2-[[1-(2-prop-2-ynoxyacetyl)-4-piperidyl]oxy]ethylamino]ethoxy]phenoxy]pyridine-3-carbonyl]amino]hexanoate

To a solution of 1-[4-(2-aminoethoxy)-1-piperidyl]-2-prop-2-ynoxy-ethanone (120 mg, 338.68 μmol, 1 eq, TFA) and 2-[3-[[5-[[(1S)-1-methoxycarbonyl-4,4-dimethyl-pentyl]carbamoyl]-2-pyridyl]oxy]phenoxy]acetic acid (189.15 mg, 338.68 μmol, 1 eq, TFA) in DMF (3 mL) was added HATU (193.16 mg, 508.01 μmol, 1.5 eq) and DIEA (131.31 mg, 1.02 mmol, 176.97 μL, 3 eq) at 0° C. Then, it was stirred at 20° C. for 1 hr. The residue was filtered. The crude product was purified by reversed-phase HPLC flash C18 gel chromatography (ISCO; 80 g SepaFlash C18 Flash Column, eluent of 0-100% MeCN/H₂O 60 ml/min) to give compound methyl (2S)-5,5-dimethyl-2-[[6-[3-[2-oxo-2-[2-[[1-(2-prop-2-ynoxyacetyl)-4-piperidyl]oxy]ethylamino]ethoxy]phenoxy]pyridine-3-carbonyl]amino]hexanoate (115 mg, 172.48 μmol, 50.93% yield) as yellow solid.

### Data:

LCMS (ESI⁺): m/z 667.4 (M+H)

### 6. General Procedure for Preparation of methyl (2S)-2-[[6-[3-[2-[2-[[1-[2-[[1-[1-[2-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxyethyl]-4-piperidyl]triazol-4-yl]methoxy]acetyl]-4-piperidyl]oxy]ethylamino]-2-oxo-ethoxy]phenoxy]pyridine-3-carbonyl]amino]-5,5-dimethyl-hexanoate

To a solution of methyl (2S)-5,5-dimethyl-2-[[6-[3-[2-oxo-2-[2-[[1-(2-prop-2-ynoxyacetyl)-4-piperidyl]oxy]ethylamino]ethoxy]phenoxy]pyridine-3-carbonyl]amino]hexanoate (55 mg, 82.49 μmol, 1 eq) and 3-[(1R)-1-[[6-[2-[2-(4-azido-1-piperidyl)ethoxy]pyrimidin-5-yl]-3-chloro-7-fluoro-2-methyl-1,5-naphthyridin-4-yl]amino]ethyl]-4-fluoro-benzonitrile (52.40 mg, 86.61 μmol, 1.05 eq) in t-BuOH (1 mL) and H₂O (1 mL) was added CuSO₄·5H₂O (10.30 mg, 41.24 μmol, 0.5 eq) and sodium; (2R)-2-[(1S)-1,2-dihydroxyethyl]-4-hydroxy-5-oxo-2H-furan-3-olate (16.34 mg, 82.49 μmol, 1 eq). Then, it was stirred at 50° C. for 1 hr. The solution was filtered to obtain the crude product. The crude product was purified by reversed-phase HPLC flash C18 gel chromatography (ISCO; 40 g SepaFlash C18 Flash Column, eluent of 0-100% MeCN/H₂O 50 ml/min) to give compound methyl (2S)-2-[[6-[3-[2-[2-[[1-[2-[[1-[1-[2-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxyethyl]-4-piperidyl]triazol-4-yl]methoxy]acetyl]-4-piperidyl]oxy]ethylamino]-2-oxo-ethoxy]phenoxy]pyridine-3-carbonyl]amino]-5,5-dimethyl-hexanoate (50 mg, 39.31 μmol, 47.66% yield) as a white solid.

### Data:

LCMS (ESI⁺): m/z 1272.7 (M+H)

### 7. General Procedure for Preparation of (2S)-2-[[6-[3-[2-[2-[[1-[2-[[1-[1-[2-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxyethyl]-4-piperidyl]triazol-4-yl]methoxy]acetyl]-4-piperidyl]oxy]ethylamino]-2-oxo-ethoxy]phenoxy]pyridine-3-carbonyl]amino]-5,5-dimethyl-hexanoic acid

the deprotection of methyl (2S)-2-[[6-[3-[2-[2-[[1-[2-[[1-[1-[2-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxyethyl]-4-piperidyl]triazol-4-yl]methoxy]acetyl]-4-piperidyl]oxy]ethylamino]-2-oxo-ethoxy]phenoxy]pyridine-3-carbonyl]amino]-5,5-dimethyl-hexanoate (50 mg, 39.31 μmol, 1 eq) in THF (2 mL) and H₂O (1 mL) was performed analogously to described above for BF083 to give the compound of the title (BF090) (19.5 mg, 15.50 μmol, 39.43% yield, 100% purity) as a white solid.

### Data:

LCMS (ESI⁺): m/z 1257.3 (M+H)

¹H NMR (400 MHZ, METHANOL-d₄) δ=9.10 (m, 2H) 8.62 (br d, J=1.22 Hz, 1H) 8.23 (m, 1H) 8.14 (s, 1H) 7.93 (d, J=11.62 Hz, 1H) 7.80 (m, 1H) 7.66 (s, 1H) 7.34 (m, 1H) 7.19 (m, 1H) 7.03 (m, 1H) 6.87 (m, 1H) 6.79 (m, 2H) 6.45 (m, 1H) 4.82 (br d, J=4.65 Hz, 4H) 4.71 (m, 1H) 4.63 (s, 2H) 4.55 (s, 2H) 4.44 (m, 1H) 4.23 (m, 2H) 3.69 (m, 1H) 3.51 (m, 9H) 3.19 (m, 2H) 2.85 (m, 2H) 2.72 (m, 3H) 2.31 (m, 4H) 1.88 (m, 4H) 1.72 (d, J=6.85 Hz, 3H) 1.48 (m, 2H) 1.34 (m, 2H) 0.89 (s, 9H)

### BF094

### 1. General Procedure for Preparation of methyl (2S)-5,5-dimethyl-2-[[6-[3-[[1-[2-[2-(2-prop-2-ynoxyethoxy)ethoxy]acetyl]-4-piperidyl]oxy]phenoxy]pyridine-3-carbonyl]amino]hexanoate

To a solution of methyl (2S)-5,5-dimethyl-2-[[6-[3-(4-piperidyloxy)phenoxy]pyridine-3-carbonyl]amino]hexanoate (0.3 g, 514.06 μmol, 1 eq, TFA), 2-[2-(2-prop-2-ynoxyethoxy) ethoxy]acetic acid (155.92 mg, 771.08 μmol, 1.5 eq) in DMF (6 mL) was added HATU (293.19 mg, 771.08 μmol, 1.5 eq) and DIEA (199.31 mg, 1.54 mmol, 268.62 μL, 3 eq) at 0° C., then the reaction was stirred for 0.5 hr at 20° C. The reaction mixture was partitioned between water 10 mL and EtOAc 10 mL. The organic phase was separated, washed with brine 15 mL (5 mL*3), dried over Na₂SO₄, filtered and concentrated under reduced pressure to give a residue. The residue was purified by flash silica gel chromatography (ISCO®; 40 g SepaFlash® Silica Flash Column, Eluent of 0˜35% Ethylacetate/Petroleum ether gradient @ 70 mL/min) to give compound methyl (2S)-5,5-dimethyl-2-[[6-[3-[[1-[2-[2-(2-prop-2-ynoxyethoxy)ethoxy]acetyl]-4-piperidyl]oxy]phenoxy]pyridine-3-carbonyl]amino]hexanoate (0.3 g, 458.88 μmol, 89.27% yield) as a yellow oil.

### Data:

LCMS (ESI⁺): m/z 654.5 (M+H)

### 2. General Procedure for Preparation of methyl (2S)-2-[[6-[3-[[1-[2-[2-[2-[[1-[2-[(2R)-4-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]-2-methyl-piperazin-1-yl]-2-oxo-ethyl]triazol-4-yl]methoxy]ethoxy]ethoxy]acetyl]-4-piperidyl]oxy]phenoxy]pyridine-3-carbonyl]amino]-5,5-dimethyl-hexanoate

The click of methyl (2S)-5,5-dimethyl-2-[[6-[3-[[1-[2-[2-(2-prop-2-ynoxyethoxy) ethoxy]acetyl]-4-piperidyl]oxy]phenoxy]pyridine-3-carbonyl]amino]hexanoate (58.18 mg, 88.99 μmol, 1.1 eq) and 3-[(1R)-1-[[6-[2-[(3R)-4-(2-azidoacetyl)-3-methyl-piperazin-1-yl]pyrimidin-5-yl]-3-chloro-7-fluoro-2-methyl-1, 5-naphthyridin-4-yl]amino]ethyl]-4-fluoro-benzonitrile (50 mg, 80.90 μmol, 1 eq) in t-BuOH (0.5 mL) was performed analogously to step 2 described in the preparation of BF083 to give compound methyl (2S)-2-[[6-[3-[[1-[2-[2-[2-[1-[2-[(2R)-4-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]-2-methyl-piperazin-1-yl]-2-oxo-ethyl]triazol-4-yl]methoxy]ethoxy]ethoxy]acetyl]-4-piperidyl]oxy]phenoxy]pyridine-3-carbonyl]amino]-5,5-dimethyl-hexanoate (60 mg, 47.18 μmol, 58.31% yield) as a white solid.

### Data:

LCMS (ESI⁺): m/z 1271.7 (M+H)

### 3. General Procedure for Preparation of (2S)-2-[[6-[3-[[1-[2-[2-[2-[[1-[2-[(2R)-4-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]-2-methyl-piperazin-1-yl]-2-oxo-ethyl]triazol-4-yl]methoxy]ethoxy]ethoxy]acetyl]-4-piperidyl]oxy]phenoxy]pyridine-3-carbonyl]amino]-5,5-dimethyl-hexanoic acid

The deprotection of methyl (2S)-2-[[6-[3-[[1-[2-[2-[2-[[1-[2-[(2R)-4-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]-2-methyl-piperazin-1-yl]-2-oxo-ethyl]triazol-4-yl]methoxy]ethoxy]ethoxy]acetyl]-4-piperidyl]oxy]phenoxy]pyridine-3-carbonyl]amino]-5,5-dimethyl-hexanoate (50 mg, 39.31 μmol, 1 eq) in THF (0.5 mL) and H₂O (0.5 mL) was performed analogously to described above for BF083 to give the compound of the title (BF094) (2.2 mg, 1.69 μmol, 21.23% yield, 96.522% purity) as a white solid.

### Data:

LCMS (ESI⁺): m/z 1257.5 (M+H)

¹H NMR (400 MHZ, METHANOL-d₄) δ=8.90 (d, J=1.4 Hz, 2H), 8.62 (d, J=1.9 Hz, 1H), 8.23 (dd, J=2.5, 8.6 Hz, 1H), 7.98 (br s, 1H), 7.86 (d, J=11.8 Hz, 1H), 7.78 (dd, J=2.1, 6.7 Hz, 1H), 7.66-7.57 (m, 1H), 7.33 (t, J=8.3 Hz, 1H), 7.25-7.14 (m, 1H), 6.98 (d, J=8.6 Hz, 1H), 6.87 (br d, J=8.0 Hz, 1H), 6.81 (s, 1H), 6.72 (br d, J=7.9 Hz, 1H), 6.46 (d, J=6.9 Hz, 1H), 5.70-5.54 (m, 1H), 5.51-5.35 (m, 1H), 4.66 (s, 2H), 4.60 (br s, 3H), 4.48 (dd, J=5.1, 8.3 Hz, 1H), 4.26 (d, J=2.0 Hz, 2H), 3.80-3.64 (m, 11H), 3.54-3.42 (m, 4H), 3.19-3.09 (m, 1H), 2.69 (s, 3H), 2.04-1.91 (m, 3H), 1.82-1.71 (m, 6H), 1.36-1.29 (m, 4H), 1.21 (br d, J=6.0 Hz, 2H), 0.89 (s, 9H)

### BF078, BF077, BF076

### 1. General Procedure for Preparation of methyl (2S)-5,5-dimethyl-2-[[6-[3-[2-oxo-2-[2-(2-prop-2-ynoxyethoxy)ethylamino]ethoxy]phenoxy]pyridine-3-carbonyl]amino]hexanoate

To a solution of 2-[3-[[5-[[(1S)-1-methoxycarbonyl-4,4-dimethyl-pentyl]carbamoyl]-2-pyridyl]oxy]phenoxy]acetic acid (100.00 mg, 179.05 μmol, 1 eq, TFA), 2-(2-prop-2-ynoxyethoxy) ethanamine (30.76 mg, 214.86 μmol, 1.2 eq) in DMF (3 mL) was added HATU (102.12 mg, 268.58 μmol, 1.5 eq) at 0° C., then DIEA (69.42 mg, 537.15 μmol, 93.56 uL, 3 eq) was added. The mixture was stirred at 20° C. for 2 hr. The reaction mixture was diluted with H₂O 3 mL, and extracted with Ethyl acetate 9 mL (3 mL*3). The combined organic layers were washed with brine 9 mL (3 mL*3), dried over Na₂SO₄, filtered and concentrated under reduced pressure. The residue was purified by prep-TLC (SiO₂, Petroleum ether:Ethyl acetate=0:1) to give methyl (2S)-5, 5-dimethyl-2-[[6-[3-[2-oxo-2-[2-(2-prop-2-ynoxyethoxy)ethylamino]ethoxy]phenoxy]pyridine-3-carbonyl]amino]hexanoate (110 mg, 193.10 μmol, 53.92% yield) as a colorless oil.

### Data:

LCMS (ESI⁺): m/z 570.3 (M+H)

### 2. General Procedure for Preparation of methyl (2S)-2-[[6-[3-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethylamino]-2-oxo-ethoxy]phenoxy]pyridine-3-carbonyl]amino]-5,5-dimethyl-hexanoate

of the click reaction of methyl (2S)-5,5-dimethyl-2-[[6-[3-[2-oxo-2-[2-(2-prop-2-ynoxyethoxy)ethylamino]ethoxy]phenoxy]pyridine-3-carbonyl]amino]hexanoate (100 mg, 175.55 μmol, 1.1 eq) and 3-[(1R)-1-[[6-[2-(5-azidopentoxy)pyrimidin-5-yl]-3-chloro-7-fluoro-2-methyl-1,5-naphthyridin-4-yl]amino]ethyl]-4-fluoro-benzonitrile (90 mg, 159.59 umol, 1 eq) in DMSO (2 mL) was performed analogously as step 2 of the preparation of BF083 described above. The residue was purified by prep-TLC (SiO₂, Ethyl acetate:Methanol=10:1) to give methyl (2S)-2-[[6-[3-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethylamino]-2-oxo-ethoxy]phenoxy]pyridine-3-carbonyl]amino]-5,5-dimethyl-hexanoate (120 mg, 105.85 μmol, 66.33% yield) as a yellow oil.

### Data:

LCMS (ESI⁺): m/z 1133.4 (M+H)

### 3. General Procedure for Preparation of (2S)-2-[[6-[3-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethylamino]-2-oxo-ethoxy]phenoxy]pyridine-3-carbonyl]amino]-5,5-dimethyl-hexanoic acid

The deprotection of methyl (2S)-2-[[6-[3-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethylamino]-2-oxo-ethoxy]phenoxy]pyridine-3-carbonyl]amino]-5,5-dimethyl-hexanoate (120 mg, 105.85 μmol, 1 eq) was performed analogously to the description above of BF083 to give the compound of the title BF078 (30.3 mg, 27.06 μmol, 25.57% yield, 100% purity) as a yellow solid

### Data:

LCMS (ESI⁺): m/z 1119.5 (M+H)

¹H NMR (400 MHZ, METHANOL-d4) δ ppm 8.99 (d, J=1.10 Hz, 2H), 8.55 (d, J=2.57 Hz, 1H), 8.16 (dd, J=8.62, 2.51 Hz, 1H), 7.99 (s, 1H), 7.92 (d, J=11.49 Hz, 1H), 7.78 (dd, J=6.97, 2.08 Hz, 1H), 7.61 (ddd, J=8.50, 4.77, 2.14 Hz, 1H), 7.34 (t, J=8.19 Hz, 1H), 7.17 (dd, J=10.15, 8.56 Hz, 1H), 6.94 (d, J=8.56 Hz, 1H), 6.88 (dd, J=8.19, 2.20 Hz, 1H), 6.81 (t, J=2.26 Hz, 1H), 6.76 (dd, J=7.83, 1.83 Hz, 1H), 6.45 (d, J=6.97 Hz, 1H), 4.63 (s, 2H), 4.59 (s, 2H), 4.49 (t, J=6.36 Hz, 2H), 4.39-4.46 (m, 3H), 3.61-3.70 (m, 6H), 3.52-3.57 (m, 2H), 2.71 (s, 3H), 1.97-2.05 (m, 2H), 1.85-1.93 (m, 3H), 1.68-1.76 (m, 4H), 1.47-1.56 (m, 2H), 1.14-1.22 (m, 2H), 0.85 (s, 9H).

The following compounds were prepared according to the general procedure. The starting materials are either commercially available or may be prepared from commercially available reagents using conventional reactions well known in the art.

### BF077

### (2S)-2-[[6-[3-[2-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethoxy]ethoxy]ethylamino]-2-oxo-ethoxy]phenoxy]pyridine-3-carbonyl]amino]-5,5-dimethyl-hexanoic acid

LC/MS [M+1]: 1295.6

¹H NMR (400 MHZ, METHANOL-d4) δ ppm 8.99 (s, 2H), 8.58 (d, J=2.38 Hz, 1H), 8.20 (dd, J=8.70, 2.50 Hz, 1H), 8.00 (s, 1H), 7.91 (d, J=11.44 Hz, 1H), 7.78 (dd, J=6.85, 1.97 Hz, 1H), 7.61 (ddd, J=8.43, 4.74, 2.09 Hz, 1H), 7.34 (t, J=8.17 Hz, 1H), 7.18 (dd, J=10.07, 8.76 Hz, 1H), 6.97 (d, J=8.58 Hz, 1H), 6.88 (dd, J=8.34, 2.38 Hz, 1H), 6.81 (t, J=2.21 Hz, 1H), 6.76 (dd, J=8.05, 1.97 Hz, 1H), 6.44 (q, J=7.07 Hz, 1H), 4.62 (s, 2H), 4.59 (s, 2H), 4.44-4.52 (m, 4H), 4.42 (dd, J=8.29, 5.07 Hz, 1H), 3.60-3.66 (m, 10 H), 3.52-3.60 (m, 14H), 2.70 (s, 3H), 2.02 (quin, J=7.36 Hz, 2H), 1.84-1.95 (m, 3H), 1.66-1.77 (m, 4H), 1.48-1.57 (m, 2H), 1.19 (dd, J=9.72, 7.57 Hz, 2H), 0.85 (s, 9H).

### BF076

### (2S)-2-[[6-[3-[2-[2-[2-[2-[2-[[1-[5-[5-[7-chloro-8-[[(1R)-1-(5-cyano-2-fluoro- phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethylamino]-2-oxo-ethoxy]phenoxy]pyridine-3-carbonyl]amino]-5,5-dimethyl-hexanoic acid

LC/MS [M+1]: 1207.6

¹H NMR (400 MHZ, METHANOL-d4) δ ppm 8.97 (d, J=1.07 Hz, 2H), 8.58 (d, J=2.15 Hz, 1H), 8.19 (dd, J=8.70, 2.50 Hz, 1H), 7.99 (s, 1H), 7.88 (d, J=11.56 Hz, 1H), 7.78 (dd, J=6.91, 2.03 Hz, 1H), 7.61 (ddd, J=8.49, 4.74, 2.15 Hz, 1H), 7.33 (t, J=8.23 Hz, 1H), 7.18 (dd, J=10.13, 8.58 Hz, 1H), 6.95 (d, J=8.70 Hz, 1H), 6.89 (dd, J=8.23, 2.03 Hz, 1H), 6.82 (t, J=2.26 Hz, 1H), 6.76 (dd, J=8.11, 1.55 Hz, 1H), 6.42 (q, J=6.83 Hz, 1H), 4.60 (s, 2H), 4.56 (s, 2H), 4.42-4.50 (m, 4H), 4.36 (dd, J=7.39, 4.89 Hz, 1H), 3.57-3.65 (m, 14H), 3.51-3.55 (m, 2H), 2.68 (s, 3H), 2.01 (quin, J=7.36 Hz, 2H), 1.83-1.94 (m, 3H), 1.71 (d, J=6.79 Hz, 4H), 1.47-1.57 (m, 2H), 1.17 (dd, J=9.66, 7.51 Hz, 2H), 0.83 (s, 9H).

### BF101, BF098

### 1. General Procedure for Preparation of 3-[[6-[2-[(3R)-4-(2-azidoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile

To a solution of 3-[[2,2-dimethyl-6-[2-[(3R)-3-methylpiperazin-1-yl]pyrimidin-5-yl]-3-oxo-pyrrolo[2,3-b]pyridin-1-yl]methyl]pyridine-2-carbonitrile (250 mg, 439.72 μmol, 1 eq, TFA), 2-azidoacetic acid (53.33 mg, 527.66 μmol, 1.2 eq) in DMF (5 mL) was added HATU (334.39 mg, 879.43 μmol, 2 eq) and DIEA (113.66 mg, 879.43 μmol, 153.18 uL, 2 eq) at 0° C. The mixture was stirred at 25° C. for 2 hr. The reaction mixture diluted with H₂O 10 mL, and extracted with EA 30 mL (10 mL*3). The combined organic layers were washed with brine 30 mL (10 mL*3), dried over Na₂SO₄, filtered and concentrated under reduced pressure. The residue was purified by prep-TLC (SiO₂, PE:EA=0:1) to give compound 3-[[6-[2-[(3R)-4-(2-azidoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (230 mg, 427.85 μmol, 97.30% yield) as a yellow oil.

### Data:

LCMS (ESI⁺): m/z 538.2 (M+1)

### 2. General Procedure for Preparation of methyl 6-(3-hydroxyphenoxy)pyridine-3-carboxylate

To a solution of methyl 6-(3-benzyloxyphenoxy)pyridine-3-carboxylate (3.3 g, 9.84 mmol, N/A purity, 1 eq) in THF (50 mL) was added Pd/C (3 g, 196.81 mmol, 10% purity, 20 eq) under N₂ atmosphere, then the reaction was degassed and purged with H₂ (15 psi) for 3 times. The mixture was stirred at 15° C. for 2 hr under H₂ (15 psi) atmosphere. The reaction mixture was filtered and filtrate was concentrated to give a residue which was purified by column chromatography (SiO₂, Petroleum ether/Ethyl acetate=1/0 to 0/1) to give compound methyl 6-(3-hydroxyphenoxy)pyridine-3-carboxylate (1.95 g, 7.95 mmol, 80.81% yield, N/A purity) as a white solid.

### 3. General Procedure for Preparation of 2-[2-[2-(2-prop-2-ynoxyethoxy)ethoxy]ethoxy]ethyl 4-methylbenzenesulfonate

To a solution of 2-[2-[2-(2-prop-2-ynoxyethoxy)ethoxy]ethoxy]ethanol (500 mg, 2.15 mmol, 1 eq) in DCM (5 mL) was added TosCl (820.79 mg, 4.31 mmol, 2 eq) and TEA (435.65 mg, 4.31 mmol, 599.24 uL, 2 eq) at 0° C. The mixture was stirred at 25° C. for 2 hr. The reaction mixture was quenched by addition H₂O 5 mL, and extracted with EA 15 mL (5 mL*3). The combined organic layers dried over Na₂SO₄, filtered and concentrated under reduced pressure to give a residue which was purified by column chromatography (SiO₂, Petroleum ether/Ethyl acetate=1/0 to 0/1) to give compound 2-[2-[2-(2-prop-2-ynoxyethoxy)ethoxy]ethoxy]ethyl 4-methylbenzenesulfonate (570 mg, 1.44 mmol, 66.69% yield, 97.33% purity) as a colorless oil.

### Data:

LCMS (ESI⁺): m/z 387.1 (M+1)

### 4. General Procedure for Preparation of methyl 6-[3-[2-[2-[2-(2-prop-2-ynoxyethoxy)ethoxy]ethoxy]ethoxy]phenoxy]pyridine-3-carboxylate

To a solution of methyl 6-(3-hydroxyphenoxy)pyridine-3-carboxylate (200 mg, 815.56 umol, 1 eq), 2-[2-[2-(2-prop-2-ynoxyethoxy)ethoxy]ethoxy]ethyl 4-methylbenzenesulfonate (315.18 mg, 815.56 umol, 1 eq) in DMF (4 mL) was added Cs₂CO₃ (797.17 mg, 2.45 mmol, 3 eq). The mixture was stirred at 80° C. for 2 hr. The reaction mixture was diluted with H₂O 10 mL and extracted with EA 30 mL (10 mL*3). The combined organic layers were washed with brine 30 mL (10 mL*3), dried over Na₂SO₄, filtered and concentrated under reduced pressure to give a residue which was purified by prep-TLC (SiO2, PE:EA=1:1) to give methyl 6-[3-[2-[2-[2-(2-prop-2-ynoxyethoxy)ethoxy]ethoxy]ethoxy]phenoxy]pyridine-3-carboxylate (350 mg, 761.72 umol, 93.40% yield) as a colorless oil.

### Data:

LCMS (ESI⁺): m/z 460.2 (M+1)

### 5. General Procedure for Preparation of methyl 6-[3-[2-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]ethoxy]ethoxy]phenoxy]pyridine-3-carboxylate

Sodium; (2R)-2-[(1S)-1,2-dihydroxyethyl]-4-hydroxy-5-oxo-2H-furan-3-olate (43.11 mg, 217.63 umol, 1 eq) and CuSO₄·5H₂O (21.17 mg, 108.82 umol, 16.70 uL, 0.5 eq) was added to a solution of 3-[[6-[2-[(3R)-4-(2-azidoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (116.99 mg, 217.63 umol, 1 eq) and methyl 6-[3-[2-[2-[2-(2-prop-2-ynoxyethoxy)ethoxy]ethoxy]ethoxy]phenoxy]pyridine-3-carboxylate (100 mg, 217.63 umol, 1 eq) in t-BuOH (1 mL) and H₂O (1 mL). The mixture was stirred at 50° C. for 1 hr. The reaction mixture was diluted with H₂O 5 mL, and extracted with EA 15 mL (5 mL*3). The combined organic layers were washed with brine 30 mL (10 mL*3), dried over Na₂SO₄, filtered and concentrated under reduced pressure to give a residue which was purified by prep-TLC (SiO₂, Ethyl acetate:Methanol=5:1) to give compound methyl 6-[3-[2-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]ethoxy]ethoxy]phenoxy]pyridine-3-carboxylate (150 mg, 150.44 umol, 69.13% yield) as a yellow oil.

### Data:

LCMS (ESI⁺): m/z 997.4 (M+1)

### 6. General Procedure for Preparation of 6-[3-[2-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]ethoxy]ethoxy]phenoxy]pyridine-3-carboxylic acid

To a solution of methyl 6-[3-[2-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]ethoxy]ethoxy]phenoxy]pyridine-3-carboxylate (130 mg, 130.38 umol, 1 eq) in THF (1.5 mL) and H₂O (0.5 mL) was added LiOH·H₂O (10.94 mg, 260.77 umol, 2 eq). The mixture was stirred at 25° C. for 12 hr. 1M HCl was added to the reaction until pH=2. The reaction mixture was extracted with EA 50 mL (5 mL*10). The combined organic layers were dried over Na₂SO₄, filtered and concentrated under reduced pressure to give compound 6-[3-[2-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]ethoxy]ethoxy]phenoxy]pyridine-3-carboxylic acid (130 mg, crude, HCl) as a yellow oil, which was used to next step without purification.

### Data:

LCMS (ESI⁺): m/z 983.4 (M+1)

### 7. General Procedure for Preparation of (2,5-dioxopyrrolidin-1-yl) 6-[3-[2-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]ethoxy]ethoxy]phenoxy]pyridine-3-carboxylate

To a solution of 6-[3-[2-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]ethoxy]ethoxy]phenoxy]pyridine-3-carboxylic acid (25 mg, 25.43 umol, 1 eq), 1-hydroxypyrrolidine-2,5-dione (14.63 mg, 127.16 umol, 5 eq) in DMF (1 mL) was added EDCI (14.63 mg, 76.29 umol, 3 eq) at 0° C. The mixture was stirred at 25° C. for 1 hr. The reaction mixture was diluted with H₂O 1 mL, and extracted with EA 3 mL (1 mL*3). The combined organic layers were washed with brine 3 mL (1 mL*3), dried over Na₂SO₄, filtered and concentrated under reduced pressure to give compound (2,5-dioxopyrrolidin-1-yl) 6-[3-[2-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]ethoxy]ethoxy]phenoxy]pyridine-3-carboxylate (25 mg, crude) as a yellow oil, which was used to next step without purification.

### Data:

LCMS (ESI⁺): m/z 1080.4 (M+1)

### 8. General Procedure for Preparation of (2S)-2-[[6-[3-[2-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]ethoxy]ethoxy]phenoxy]pyridine-3-carbonyl]amino]-5,5-dimethyl-hexanoic acid

To a solution of (2,5-dioxopyrrolidin-1-yl) 6-[3-[2-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]ethoxy]ethoxy]phenoxy]pyridine-3-carboxylate (8 mg, 7.41 umol, 1 eq), (2S)-2-amino-5,5-dimethyl-hexanoic acid (1.18 mg, 7.41 umol, 1 eq) in DMF (0.6 mL), DCM (0.1 mL) and H₂O (0.3 mL) was added DIEA (1.91 mg, 14.81 umol, 2.58 uL, 2 eq). The mixture was stirred at 25° C. for 1 hr. The mixture was filtered and the filtrate was purified by prep-HPLC (basic condition column: Waters Xbridge Prep OBD C18 150*40 mm*10 um; mobile phase: [water (NH₃H₂O+NH₄HCO₃)-ACN]; B %: 30%-60%, 8 min) to give the compound of the title BF101 (16 mg, 14.23 umol, 64.05% yield, 100% purity) as a yellow solid.

### Data:

LCMS (ESI⁺): m/z 1124.5 (M+1)

¹H NMR (400 MHz, METHANOL-d4) δ=8.95 (s, 2H), 8.62 (d, J=2.8 Hz, 2H), 8.23 (dd, J=2.5, 8.6 Hz, 1H), 8.03 (d, J=7.4 Hz, 1H), 7.95 (d, J=8.0 Hz, 2H), 7.62 (dd, J=4.7, 8.1 Hz, 1H), 7.34-7.29 (m, 2H), 6.97 (d, J=8.5 Hz, 1H), 6.84 (dd, J=1.7, 8.3 Hz, 1H), 6.77-6.70 (m, 2H), 5.66-5.36 (m, 2H), 5.12 (s, 2H), 4.75-4.69 (m, 2H), 4.65 (s, 2H), 4.60-4.54 (m, 2H), 4.49 (dd, J=5.3, 8.5 Hz, 1H), 4.35 (br dd, J=3.5, 8.3 Hz, 1H), 4.15-4.06 (m, 2H), 3.84-3.80 (m, 2H), 3.73-3.57 (m, 14H), 3.16-3.07 (m, 1H), 2.02-1.90 (m, 1H), 1.85-1.76 (m, 1H), 1.42-1.26 (m, 11H), 0.90 (s, 9H)

The following compounds were prepared according to the general procedure.

The starting materials are either commercially available or may be prepared from commercially available reagents using conventional reactions well known in the art.

### BF098

### (2S)-2-[[6-[3-[2-[2-[2-[2-[2-[[1-[2-[(2R)-4-[5-[1-[(2-cyano-3-pyridyl)methyl]-2,2- dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]ethoxy]ethoxy]ethoxy]phenoxy]pyridine-3-carbonyl]amino]-5,5-dimethyl-hexanoic acid

LC/MS [M+1] 1168.5

¹H NMR (400 MHz, METHANOL-d4) δ=8.95 (s, 2H), 8.64-8.59 (m, 2H), 8.22 (dd, J=2.5, 8.7 Hz, 1H), 8.03 (dd, J=1.4, 8.1 Hz, 1H), 7.97 (br s, 1H), 7.95 (d, J=8.1 Hz, 1H), 7.61 (dd, J=4.7, 8.2 Hz, 1H), 7.35-7.28 (m, 2H), 6.97 (d, J=8.7 Hz, 1H), 6.87-6.82 (m, 1H), 6.76 (t, J=2.3 Hz, 1H), 6.72 (td, J=1.1, 8.0 Hz, 1H), 5.69-5.53 (m, 1H), 5.49-5.36 (m, 1H), 5.12 (s, 2H), 4.76-4.68 (m, 2H), 4.65 (s, 2H), 4.50 (dd, J=5.1, 8.8 Hz, 1H), 4.42-4.29 (m, 1H), 4.11 (dd, J=3.8, 5.4 Hz, 2H), 3.82 (dd, J=3.8, 5.4 Hz, 2H), 3.71-3.53 (m, 18H), 3.42-3.34 (m, 1H), 3.17-3.04 (m, 1H), 2.01-1.90 (m, 1H), 1.86-1.75 (m, 1H), 1.42-1.25 (m, 11H), 0.91 (s, 9H)

### BF100

### 1. General Procedure for Preparation of methyl 6-[2-[2-(tert-butoxycarbonylamino)ethoxy]ethoxy]hexanoate

To a solution of tert-butyl N-[2-(2-hydroxyethoxy)ethyl]carbamate (400 mg, 1.95 mmol) in DMF (8 mL) was added NaH (116.92 mg, 2.92 mmol, 60% purity) at 0° C. and the mixture was stirred for 1 hr. The mixture was add methyl 6-bromohexanoate (448.21 mg, 2.14 mmol) and NaI (29.21 mg, 194.88 umol) and stirred at 55° C. for 11 hr. TLC indicated Reactant 1 was remained, and three major new spot with lower polarity were detected. The reaction mixture was quenched with NH₄Cl 15 mL and extracted with EA 30 mL (10 mL*3). The combined organic layers were dried over anhydrous Na₂SO₄, filtered and concentrated under reduced pressure to give a residue. The residue was purified by prep-TLC (SiO₂, Petroleum ether: Ethyl acetate=1:1) to give methyl 6-[2-[2-(tert-butoxycarbonylamino)ethoxy]ethoxy]hexanoate (70 mg, 209.95 umol, 10.77% yield) as a yellow oil.

### Data:

¹H NMR (400 MHz, CHLOROFORM-d) δ ppm 3.67 (s, 3H) 3.54-3.62 (m, 6H) 3.44-3.49 (m, 2H) 3.32 (br d, J=5.14 Hz, 2H) 2.27-2.38 (m, 2H) 1.64 (dt, J=14.86, 7.37 Hz, 6H) 1.45 (s, 9H)

### 2. General Procedure for Preparation of 3-[[6-[2-[(3R)-4-(2-aminoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile

To a solution of methyl 6-[2-[2-(tert-butoxycarbonylamino)ethoxy]ethoxy]hexanoate (140 mg, 419.89 umol) in THF (0.5 mL) and H₂O (0.5 mL) was added LiOH·H₂O (35.24 mg, 839.78 umol). The mixture was stirred at 20° C. for 1 hr. The mixture was diluted with water 10 mL, and then extracted with Ethyl acetate 45 mL (15 mL*3). The mixture was added HCl (1M) to pH=2, and then extracted with Ethyl acetate 15 mL (5 mL*3). The combined organic layers were washed with sat. NaCl 10 mL, dried over Na₂SO₄, filtered and concentrated under reduced pressure to give compound 6-[2-[2-(tert-butoxycarbonylamino)ethoxy]ethoxy]hexanoic acid (120 mg, crude) as a yellow oil.

### 3. General Procedure for Preparation of tert-butyl N-[2-[2-[6-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-6-oxo-hexoxy]ethoxy]ethyl]carbamate

To a solution of 6-[2-[2-(tert-butoxycarbonylamino)ethoxy]ethoxy]hexanoic acid (112.32 mg, 351.66 umol) and 3-[[6-[2-[(3R)-4-(2-aminoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (220 mg, 351.66 umol, TFA) in DMF (3 mL) was added HATU (200.57 mg, 527.49 umol) and DIEA (90.90 mg, 703.32 umol, 122.51 uL) at 0° C. The mixture was stirred at 20° C. for 1 hr. The mixture was diluted with water 5 mL, and then extracted with Ethyl acetate 15 mL (5 mL*3). The combined organic layers were washed with sat·NaCl 10 mL, dried over Na₂SO₄, filtered and concentrated under reduced pressure to give a residue. The residue was purified by prep-TLC (SiO₂, Ethyl acetate:Methanol=10:1) to give compound tert-butyl N-[2-[2-[6-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-6-oxo-hexoxy]ethoxy]ethyl]carbamate (120 mg, 147.61 umol, 41.97% yield) as a yellow oil

### Data:

LCMS (ESI⁺): m/z 813.4 (M+H)

### 4. General Procedure for Preparation of 6-[2-(2-aminoethoxy)ethoxy]-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]hexanamide

A solution of tert-butyl N-[2-[2-[6-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-6-oxo-hexoxy]ethoxy]ethyl]carbamate (120 mg, 147.61 umol) in DCM (1 mL) and TFA (0.2 mL) was stirred at 20° C. for 1 hr. The DCM and TFA was removed under reduced pressure to give compound 6-[2-(2-aminoethoxy)ethoxy]-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]hexanamide (120 mg, crude, TFA) as a yellow oil.

### Data:

LCMS (ESI+): m/z 713.3 (M+H)

### 5. General Procedure for Preparation of methyl (2S)-2-[[6-[3-[2-[2-[6-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-6-oxo-hexoxy]ethoxy]ethylcarbamoyl]phenoxy]pyridine-3-carbonyl]amino]-5,5-dimethyl-hexanoate

To a solution of 6-[2-(2-aminoethoxy)ethoxy]-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]hexanamide (110 mg, 133.03 umol, TFA) and 3-[[5-[[(1S)-1-methoxycarbonyl-4,4-dimethyl-pentyl]carbamoyl]-2-pyridyl]oxy]benzoic acid (84.37 mg, 159.64 umol, TFA) in DMF (2 mL) was added DIEA (34.39 mg, 266.07 umol, 46.34 uL) and HATU (75.87 mg, 199.55 umol) at 0° C. The mixture was stirred at 20° C. for 1 hr. The mixture was diluted with water 5 mL, and then extracted with Ethyl acetate 15 mL (5 mL*3). The combined organic layers were washed with sat·NaCl 10 mL, dried over Na₂SO₄, filtered and concentrated under reduced pressure to give a residue. The residue was purified by prep-TLC (SiO2,Ethyl acetate:Methanol=10:1) to give compound methyl (2S)-2-[[6-[3-[2-[2-[6-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-6-oxo-hexoxy]ethoxy]ethylcarbamoyl]phenoxy]pyridine-3-carbonyl]amino]-5,5-dimethyl-hexanoate (50 mg, 45.07 umol, 33.88% yield) as a yellow oil.

### Data:

LCMS (ESI⁺): m/z 1109.5 (M+H)

### 6. General Procedure for Preparation of (2S)-2-[[6-[3-[2-[2-[6-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-6-oxo-hexoxy]ethoxy]ethylcarbamoyl]phenoxy]pyridine-3-carbonyl]amino]-5,5-dimethyl-hexanoic acid

To a solution of methyl (2S)-2-[[6-[3-[2-[2-[6-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-6-oxo-hexoxy]ethoxy]ethylcarbamoyl]phenoxy]pyridine-3-carbonyl]amino]-5,5-dimethyl-hexanoate (50 mg, 45.07 umol) in THF (0.5 mL) and H₂O (0.5 mL) was added LiOH·H₂O (3.78 mg, 90.15 umol). The mixture was stirred at 20° C. for 1 hr. The THF was removed under reduced pressure and the residue was acid with FA to pH=2. The mixture was filtered and the filtrate was purified by prep-HPLC (F A condition; column: Phenomenex Luna C18 200*40 mm*10 um; mobile phase: [water (FA)-ACN]; B %: 40%-75%, 8 min) to give compound (2S)-2-[[6-[3-[2-[2-[6-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-6-oxo-hexoxy]ethoxy]ethylcarbamoyl]phenoxy]pyridine-3-carbonyl]amino]-5,5-dimethyl-hexanoic acid (14.4 mg, 12.94 umol, 28.70% yield, 98.395% purity) as a yellow solid.

### Data:

LCMS (ESI⁺): m/z 1095.5 (M+H)

¹H NMR (400 MHz, METHANOL-d4) δ ppm 8.94 (s, 2H) 8.60-8.65 (m, 2H) 8.27 (dd, J=8.58, 2.50 Hz, 1H) 8.03 (d, J=8.11 Hz, 1H) 7.94 (d, J=7.99 Hz, 1H) 7.74 (d, J=7.75 Hz, 1H) 7.64 (t, J=1.85 Hz, 1H) 7.61 (dd, J=8.11, 4.77 Hz, 1H) 7.54 (t, J=7.93 Hz, 1H) 7.34 (dd, J=7.75, 1.91 Hz, 1H) 7.30 (d, J=8.11 Hz, 1H) 7.07 (d, J=8.70 Hz, 1H) 5.12 (s, 2H) 4.60-4.71 (m, 3H) 4.51 (br dd, J=8.46, 5.36 Hz, 1H) 4.21-4.41 (m, 1H) 4.10-4.19 (m, 1H) 3.96-4.09 (m, 1H) 3.60-3.68 (m, 4H) 3.54-3.60 (m, 4H) 3.47 (t, J=6.50 Hz, 2H) 3.34 (br s, 2H) 3.01-3.15 (m, 1H) 2.26 (t, J=7.45 Hz, 2H) 1.90-2.02 (m, 1H) 1.75-1.88 (m, 1H) 1.52-1.66 (m, 4H) 1.30-1.41 (m, 10H) 1.10-1.27 (m, 3H) 0.91 (s, 9H)

### BF106

### 1. General Procedure for Preparation of 6-(tert-butoxycarbonylamino)hexyl 4-methylbenzenesulfonate

To a solution of tert-butyl N-(6-hydroxyhexyl) carbamate (1 g, 4.60 mmol) in DCM (10 mL) was added TEA (931.31 mg, 9.20 mmol, 1.28 mL) and TosCl (1.75 g, 9.20 mmol) at 0° C. The mixture was stirred at 20° C. for 2 hr. The mixture was diluted with water 10 mL, and then extracted with Ethyl acetate 60 mL (20 mL*3). The combined organic layers were washed with sat. NaCl 10 mL*2, dried over Na₂SO₄, filtered and concentrated under reduced pressure to give a residue. The residue was purified by flash silica gel chromatography (ISCO®; 40 g SepaFlash® Silica Flash Column, Eluent of 0˜50% Ethyl acetate/Petroleum ether gradient @ 80 mL/min) to give compound 6-(tert-butoxycarbonylamino)hexyl 4-methylbenzenesulfonate (1.7 g, 4.58 mmol, 99.44% yield) as a white solid.

### 2. General Procedure for Preparation of tert-butyl N-[6-(2-hydroxyethoxy)hexyl]carbamate

A solution of NaH (516.84 mg, 12.92 mmol, 60% purity) in ethane-1,2-diolate (12 mL) was stirred at 0° C. for 0.5 hr. 6-(tert-butoxycarbonylamino)hexyl 4-methylbenzenesulfonate (1.2 g, 3.23 mmol) was added to the above solution, the mixture was stirred at 65° C. for 1.5 hr. The mixture was quenched with saturated NH₄Cl solution (20 mL) at 0° C. and extracted with Ethyl acetate 60 mL (20 mL*3). The organic layer was dried over anhydrous Na₂SO₄, filtered and concentrated under reduced pressure to give a residue. The residue was purified by flash silica gel chromatography (ISCO®; 40 g SepaFlash® Silica Flash Column, Eluent of 0˜50% Ethyl acetate/Petroleum ether gradient @ 80 mL/min) to give compound tert-butyl N-[6-(2-hydroxyethoxy)hexyl]carbamate (840 mg, 3.21 mmol, 99.50% yield) as a colorless oil.

### 3. General Procedure for Preparation of methyl 2-[2-[6-(tert-butoxycarbonylamino) hexoxy]ethoxy]acetate

To a stirred solution of tert-butyl N-[6-(2-hydroxyethoxy)hexyl]carbamate (500 mg, 1.91 mmol) in DMF (9 mL) was added NaH (114.77 mg, 2.87 mmol, 60% purity) at 0° C., then the reaction was stirred for 0.5 hr at 0° C., then methyl 2-bromoacetate (292.65 mg, 1.91 mmol, 180.65 uL) and NaI (28.68 mg, 191.31 umol) was added at 0° C., then the reaction was stirred for 11.5 hr at 55° C. The mixture was quenched with saturated NH₄Cl solution (20 mL) at 0° C. and extracted with Ethyl acetate 60 mL (20 mL*3). The combined organic layers were washed with sat·NaCl 15 mL*2, dried over Na₂SO₄, filtered and concentrated under reduced pressure to give a residue. The residue was purified by prep-TLC (SiO₂, Petroleum ether: Ethyl acetate=1:1) to give compound methyl 2-[2-[6-(tert-butoxycarbonylamino) hexoxy]ethoxy]acetate (180 mg, 539.86 μmol, 28.22% yield) as a colorless oil.

### 4. General Procedure Preparation of 2-[2-[6-(tert-butoxycarbonylamino)hexoxy]ethoxy]acetic acid

To a solution of methyl 2-[2-[6-(tert-butoxycarbonylamino) hexoxy]ethoxy]acetate (153.54 mg, 460.50 umol) in THF (1 mL) and H₂O (1 mL) was added LiOH·H₂O (38.65 mg, 921.01 umol). The mixture was stirred at 20° C. for 1 hr. The aqueous layer was adjusted pH to 3-4 with 1M aq·HCl, and then extracted with Ethyl acetate 15 mL (5 mL*3). The combined organic layers were washed with sat·NaCl 10 mL, dried over Na₂SO₄, filtered and concentrated under reduced pressure to give compound 2-[2-[6-(tert-butoxycarbonylamino) hexoxy]ethoxy]acetic acid (110 mg, 344.40 μmol, 74.79% yield) as a colorless oil.

### 5. General Procedure for Preparation of tert-butyl N-[6-[2-[2-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-2-oxo-ethoxy]ethoxy]hexyl]carbamate

To a solution of 3-[[6-[2-[(3R)-4-(2-aminoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (184.12 mg, 294.31 μmol, TFA) and 2-[2-[6-(tert-butoxycarbonylamino) hexoxy]ethoxy]acetic acid (94.00 mg, 294.31 μmol) in DMF (3 mL) was added HATU (223.81 mg, 588.62 μmol) and DIEA (57.05 mg, 441.46 μmol, 76.89 μL) at 0° C. The mixture was stirred at 20° C. for 1 hr. The mixture was diluted with water 5 mL, and then extracted with Ethyl acetate 15 mL (5 mL*3). The combined organic layers were washed with sat. NaCl 10 mL, dried over Na₂SO₄, filtered and concentrated under reduced pressure to give a residue. The residue was purified by prep-TLC (SiO₂, Ethyl acetate:Methanol=10:1) to give compound tert-butyl N-[6-[2-[2-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-2-oxo-ethoxy]ethoxy]hexyl]carbamate (101 mg, 124.24 μmol, 42.21% yield) as a yellow oil.

### Data:

LCMS (ESI⁺): m/z 813.4 (M+H)

### 6. General Procedure for Preparation of 2-[2-(6-aminohexoxy)ethoxy]-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]acetamide

A mixture of tert-butyl N-[6-[2-[2-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-2-oxo-ethoxy]ethoxy]hexyl]carbamate (138 mg, 169.75 μmol) in DCM (1 mL) and TFA (1 mL) was stirred at 20° C. for 1 hr. The DCM and TFA was removed under reduced pressure to give compound 2-[2-(6-aminohexoxy)ethoxy]-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]acetamide (120 mg, 145.13 μmol, 85.49% yield, TFA) as a yellow solid.

### Data:

LCMS (ESI⁺): m/z 713.4 (M+H)

### 7. General Procedure for Preparation of methyl (2S)-2-[[6-[3-[6-[2-[2-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-2-oxo-ethoxy]ethoxy]hexylcarbamoyl]phenoxy]pyridine-3-carbonyl]amino]-5,5-dimethyl-hexanoate

To a solution of 3-[[5-[[(1S)-1-methoxycarbonyl-4,4-dimethyl-pentyl]carbamoyl]-2-pyridyl]oxy]benzoic acid (70.30 mg, 133.03 μmol, TFA) and 2-[2-(6-aminohexoxy)ethoxy]-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]acetamide (110 mg, 133.03 μmol, TFA) in DMF (2 mL) was added HATU (75.87 mg, 199.55 μmol) and DIEA (34.39 mg, 266.07 μmol, 46.34 μL) at 0° C., then the reaction was stirred for 1 hr at 25° C. The mixture was diluted with water 5 mL, and then extracted with Ethyl acetate 15 mL (5 mL*3). The combined organic layers were washed with sat·NaCl 10 mL, dried over Na₂SO₄, filtered and concentrated under reduced pressure to give a residue. The residue was purified by prep-TLC (SiO₂, Ethyl acetate:Methanol=10:1) to give compound methyl (2S)-2-[[6-[3-[6-[2-[2-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-2-oxo-ethoxy]ethoxy]hexylcarbamoyl]phenoxy]pyridine-3-carbonyl]amino]-5,5-dimethyl-hexanoate (57 mg, 51.38 μmol, 38.63% yield) as a yellow solid.

### Data:

LCMS (ESI⁺): m/z 1110.6 (M+H)

### 8. General Procedure for Preparation of (2S)-2-[[6-[3-[2-[2-[2-[3-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-3-oxo-propoxy]ethoxy]ethoxy]ethylcarbamoyl]phenoxy]pyridine-3-carbonyl]amino]-5,5-dimethyl-hexanoic acid

of the deprotection of methyl (2S)-2-[[6-[3-[6-[2-[2-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-2-oxo-ethoxy]ethoxy]hexylcarbamoyl]phenoxy]pyridine-3-carbonyl]amino]-5,5-dimethyl-hexanoate (57 mg, 51.38 μmol) was performed analogously to the description above of BF083 to give the compound of the title BF106 (16.3 mg, 14.63 μmol, 28.47% yield, 98.492% purity) as a yellow solid.

### Data:

LCMS (ESI⁺): m/z 1095.5 (M+H)

¹H NMR (400 MHz, METHANOL-d₄) δ=8.94 (s, 2H), 8.65-8.56 (m, 2H), 8.25 (dd, J=2.5, 8.6 Hz, 1H), 8.03 (dd, J=1.3, 8.1 Hz, 1H), 7.94 (d, J=8.0 Hz, 1H), 7.70 (d, J=8.0 Hz, 1H), 7.65-7.57 (m, 2H), 7.51 (t, J=7.9 Hz, 1H), 7.34-7.26 (m, 2H), 7.05 (d, J=8.5 Hz, 1H), 5.12 (s, 2H), 4.79-4.55 (m, 3H), 4.51 (dd, J=5.2, 8.8 Hz, 1H), 4.41-4.16 (m, 2H), 4.15-4.08 (m, 1H), 4.06 (s, 2H), 3.83-3.74 (m, 1H), 3.74-3.69 (m, 2H), 3.65-3.60 (m, 2H), 3.50 (t, J=6.4 Hz, 2H), 3.38-3.33 (m, 3H), 3.29-3.16 (m, 1H), 3.15-2.98 (m, 1H), 2.07-1.90 (m, 1H), 1.87-1.73 (m, 1H), 1.67-1.54 (m, 4H), 1.47-1.39 (m, 4H), 1.38-1.30 (m, 8H), 1.28-1.09 (m, 3H), 0.91 (s, 9H)

### BF108

### 1. General Procedure for Preparation of 3-[tert-butyl(dimethyl)silyl]oxybenzaldehyde

To a solution of 3-hydroxybenzaldehyde (5 g, 40.94 mmol, 1 eq), tert-butyl-chloro-dimethyl-silane (9.26 g, 61.41 mmol, 7.53 mL, 1.5 eq) in DCM (70 mL) was added DMAP (1.50 g, 12.28 mmol, 0.3 eq). The mixture was stirred at 25° C. for 12 hr. The reaction mixture was partitioned between water 100 mL and EtOAc 100 mL. The organic phase was separated, washed with brine 210 mL (70 mL*3), dried over Na₂SO₄, filtered and concentrated under reduced pressure to give a residue. The residue was purified by flash silica gel chromatography (ISCO®; 20 g SepaFlash® Silica Flash Column, Eluent of 0˜10% Ethyl acetate/Petroleum ether gradient @ 80 mL/min) to give compound 3-[tert-butyl(dimethyl)silyl]oxybenzaldehyde (1.65 g, 6.98 mmol, 17.05% yield) as a light yellow oil.

### Data:

LCMS (ESI+): m/z 237.1 (M+H)

### 2. General Procedure for Preparation of 1-[3-[tert-butyl(dimethyl)silyl]oxyphenyl]but-3-yn-1-ol

In a three necked round bottom flask, Aluminum (1.29 g, 47.81 mmol, 477.78 μL, 7.29 eq) was suspended in anhydrous THF (50 mL). To this slurry catalytic HgCl₂ (450 mg, 1.66 mmol, 82.72 μL, 2.53e-1 eq) was added and mixture was stirred at 25° C. for 30 min. 3-bromoprop-1-yne (5.46 g, 45.90 mmol, 3.96 mL, 7 eq) was added dropwise at 0° C. The mixture was heated to 70° C. and stirred for 1 h at 70° C. After cooling to 25° C., a solution of 3-[tert-butyl(dimethyl)silyl]oxybenzaldehyde (1.55 g, 6.56 mmol, 1 eq) dissolved in anhydrous THF (5 mL) was added dropwise to the mixture at 0° C. and the mixture was stirred for an additional 1 h at 25° C. The crude mixture was quenched with 1N aqueous HCl (40 mL) at 0° C. The reaction mixture extracted with EA 120 mL (40 mL*3). The combined organic layers were washed with brine 50 mL*3, dried over anhydrous Na₂SO₄, filtered and concentrated under reduced pressure to give a residue. The residue was purified by column chromatography (SiO₂, Petroleum ether/Ethyl acetate=40/1 to 15/1) to give compound 1-[3-[tert-butyl(dimethyl)silyl]oxyphenyl]but-3-yn-1-ol (500 mg, 1.81 mmol, 27.58% yield) as a yellow liquid.

### Data:

¹H NMR (400 MHz, CHLOROFORM-d) δ=7.22 (t, J=7.9 Hz, 1H), 6.98 (d, J=7.6 Hz, 1H), 6.89 (t, J=1.9 Hz, 1H), 6.78 (dd, J=1.6, 8.0 Hz, 1H), 4.84 (t, J=6.3 Hz, 1H), 2.66-2.60 (m, 2H), 2.08 (t, J=2.6 Hz, 1H), 0.99 (s, 9H), 0.21 (s, 6H)

### 3. General Procedure for Preparation of 3-but-3-ynylphenol

To a solution of 1-[3-[tert-butyl(dimethyl)silyl]oxyphenyl]but-3-yn-1-ol (500 mg, 1.81 mmol, 1 eq) in DCM (8 mL) was added Et₃SiH (420.62 mg, 3.62 mmol, 577.77 μL, 2 eq), then the reaction was cooled to 0° C. and BF3·Et₂O (513.40 mg, 3.62 mmol, 444.89 UL, 2 eq) was added, then the reaction was stirred for 1 hr at 0° C., another BF3·Et₂O (1.03 g, 7.23 mmol, 889.78 μL, 4 eq) was added, and the reaction was stirred for 1 hr at 0° C. and 10 hr at 20° C. To the reaction mixture was added H₂O 2 mL and the reaction was extracted with DCM 6 mL (2 mL*3). The combined organic layers were dried over anhydrous Na₂SO₄, filtered and concentrated under reduced pressure to give a residue which was then purified by prep TLC (SiO₂:PE:EA=3:1) to give compound 3-but-3-ynylphenol (20 mg, 136.81 μmol, 7.56% yield) as a light yellow oil.

### 4. General Procedure for Preparation of (methyl 6-(3-but-3-ynylphenoxy)pyridine-3-carboxylate (39 mg, crude) as a Yellow Oil

A solution of 3-but-3-ynylphenol (20 mg, 136.81 μmol, 1 eq), methyl 6-fluoropyridine-3-carboxylate (23.35 mg, 150.49 μmol, 1.1 eq) and Cs₂CO₃ (66.86 mg, 205.22 μmol, 1.5 eq) in DMF (1 mL) was stirred for 1 hr at 80° C. The reaction mixture was diluted with H₂O 2 mL and extracted with EA 6 mL (2 mL*3). The combined organic layers were washed with brine (5 mL*3), dried over anhydrous Na₂SO₄, filtered and concentrated under reduced pressure to give compound methyl 6-(3-but-3-ynylphenoxy)pyridine-3-carboxylate (39 mg, crude) as a yellow oil, which was used to next step without purification

### Data:

LCMS (ESI+): m/z 281.9 (M+H)

### 5. General Procedure for Preparation of 9-bromo-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]nonanamide

To a solution of 3-[[6-[2-[(3R)-4-(2-aminoacetyl)-3-methyl-piperazin-1-yl]pyrimidin-5-yl]-2,2-dimethyl-3-oxopyrrolo[2,3-b]pyridin-1-yl]methyl]pyridine-2-carbonitrile (200 mg, 319.69 μmol, 1 eq, TFA) and 9-bromononanoic acid (75.81 mg, 319.69 μmol, 1 eq) in DMF (0.5 mL) was added DIEA (82.63 mg, 639.39 μmol, 111.37 μL, 2 eq), HOBt (64.80 mg, 479.54 μmol, 1.5 eq) and EDCI (122.57 mg, 639.39 μmol, 2 eq) at 0° C. The mixture was stirred at 20° C. for 1 hr. The mixture was diluted with water 5 mL, and then extracted with EtOAc 15 mL (5 mL*3). The combined organic layers were washed with brine 10 mL, dried over Na₂SO₄, filtered and concentrated under reduced pressure to give a residue. The residue was purified by prep-TLC (SiO₂, Ethyl acetate:Methanol=10:1) to give compound 9-bromo-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]nonanamide (95 mg, 130.01 μmol, 40.67% yield) as a yellow oil.

### Data:

LCMS (ESI+): m/z 730.0 (M+H)

### 6. General Procedure for Preparation of 9-azido-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]nonanamide

To a solution of 9-bromo-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]nonanamide (95 mg, 130.01 μmol, 1 eq) in DMF (1 mL) was added NaN₃ (10 mg, 153.82 μmol, 1.18 eq), then the reaction was stirred for 2 hr at 20° C. The reaction mixture was quenched by addition H₂O 5 mL, Na₂CO₃ 5 mL and then extracted with EtOAc 15 mL (5 mL*3). The combined organic layers were washed with brine 10 mL (5 mL*2), dried over Na₂SO₄, filtered and concentrated under reduced pressure to give compound 9-azido-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]nonanamide (90 mg, crude) as a yellow oil.

### Data:

LCMS (ESI+): m/z 693.5 (M+H)

### 7. General Procedure for Preparation of methyl 6-[3-[2-[1-[9-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-9-oxo-nonyl]triazol-4-yl]ethyl]phenoxy]pyridine-3-carboxylate

To a solution of methyl 6-(3-but-3-ynylphenoxy)pyridine-3-carboxylate (25 mg, 88.87 μmol, 1 eq) and 9-azido-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]nonanamide (61.57 mg, 88.87 μmol, 1 eq) in t-BuOH (1 mL) and H₂O (1 mL) was added sodium; (2R)-2-[(1S)-1,2-dihydroxyethyl]-4-hydroxy-5-oxo-2H-furan-3-olate (17.61 mg, 88.87 μmol, 1 eq) and CuSO₄·5H₂O (11.1 mg, 44.44 μmol, 6.82 μL, 0.5 eq), then the reaction was stirred for 1 hr at 50° C. The reaction mixture was diluted with H₂O 2 mL, extracted with EA 6 mL (2 mL*3). The combined organic layers were dried over anhydrous Na₂SO₄, filtered and concentrated under reduced pressure to give a residue. The residue was purified by prep-TLC (SiO₂, Ethyl acetate/MeOH=10/1) to give compound methyl 6-[3-[2-[1-[9-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-9-oxo-nonyl]triazol-4-ylethyl]phenoxy]pyridine-3-carboxylate (30 mg, 30.80 μmol, 34.65% yield) as a yellow oil.

### Data:

LCMS (ESI+): m/z 974.3 (M+H)

### 8. General Procedure for Preparation of 6-[3-[2-[1-[9-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-9-oxo-nonyl]triazol-4-yl]ethyl]phenoxy]pyridine-3-carboxylic acid

To a solution of methyl 6-[3-[2-[1-[9-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-9-oxo-nonyl]triazol-4-yl]ethyl]phenoxy]pyridine-3-carboxylate (30 mg, 30.80 μmol, 1 eq) in THF (0.5 mL) and H₂O (0.5 mL) was added LiOH·H₂O (2.58 mg, 61.59 μmol, 2 eq), then the reaction was stirred for 1 hr at 25° C. THF was removed. The aqueous layer was adjusted pH to 3-4 with 1M aq·HCl and then extracted with EA 6 mL (2 mL*3). The combined organic layers were dried over anhydrous Na₂SO₄, filtered and concentrated under reduced pressure to give compound 6-[3-[2-[1-[9-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-9-oxo-nonyl]triazol-4-yl]ethyl]phenoxy]pyridine-3-carboxylic acid (30 mg, crude, HCl) as a yellow oil

### Data:

LCMS (ESI+): m/z 960.6 (M+H)

### 9. General Procedure for Preparation of 3-[[6-[2-[(3R)-4-[2-[1-(10-aminodecyl)triazol-4-yl]acetyl]-3-methyl-piperazin-1-yl]pyrimidin-5-yl]-2,2-dimethyl-3-oxo-pyrrolo[2,3-b]pyridin-1-yl]methyl]pyridine-2-carbonitrile

To a mixture of 6-[3-[2-[1-[9-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-9-oxo-nonyl]triazol-4-yl]ethyl]phenoxy]pyridine-3-carboxylic acid (30 mg, 30.10 μmol, 1 eq, HCl) in DMF (2 mL) was added HOSu (5.20 mg, 45.16 μmol, 1.5 eq) and EDCI (11.54 mg, 60.21 μmol, 2 eq), then the mixture was stirred for 0.5 hr at 25° C. The reaction mixture was diluted with H₂O 2 mL and extracted with EA 9 mL (3 mL*3). The combined organic layers were washed with brine (10 mL*3), dried over anhydrous Na₂SO₄, filtered and concentrated under reduced pressure to give compound (2,5-dioxopyrrolidin-1-yl) 6-[3-[2-[1-[9-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-9-oxo-nonyl]triazol-4-yl]ethyl]phenoxy]pyridine-3-carboxylate (32 mg, crude) as a yellow oil.

### Data:

LCMS (ESI+): m/z 1057.3 (M+H)

### 10. General Procedure for Preparation of (2S)-2-[[6-[3-[2-[1-[9-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-9-oxo-nonyl]triazol-4-yl]ethyl]phenoxy]pyridine-3-carbonyl]amino]-5,5-dimethyl-hexanoic acid

To a solution of (2S)-2-amino-5,5-dimethyl-hexanoic acid (4.82 mg, 30.27 μmol, 1 eq) and (2,5-dioxopyrrolidin-1-yl) 6-[3-[2-[1-[9-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-9-oxo-nonyl]triazol-4-yl]ethyl]phenoxy]pyridine-3-carboxylate (32 mg, 30.27 μmol, 1 eq) in DMF (0.5 mL), DCM (0.08 mL) and H₂O (0.25 mL) was added DIEA (7.82 mg, 60.54 μmol, 10.54 μL, 2 eq). The mixture was stirred at 25° C. for 2 hr. FA was added to the reaction until pH=7. The mixture was filtered and the filtrate was purified by prep-HPLC (neutral condition; column: Waters Xbridge Prep OBD C18 150*40 mm*10 um; mobile phase: [water (NH₄HCO₃)-ACN]; B %: 30%-60%, 8 min) o give compound (2S)-2-[[6-[3-[2-[1-[9-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-9-oxo-nonyl]triazol-4-yl]ethyl]phenoxy]pyridine-3-carbonyl]amino]-5,5-dimethyl-hexanoic acid (5.7 mg, 4.97 μmol, 16.43% yield, 96.084% purity) as a yellow solid.

### Data:

LCMS (ESI+): m/z 1101.5 (M+H)

¹H NMR (400 MHz, METHANOL-d4) δ=8.95 (s, 2H), 8.65-8.60 (m, 2H), 8.24 (dd, J=2.5, 8.7 Hz, 1H), 8.03 (dd, J=1.4, 8.0 Hz, 1H), 7.95 (d, J=8.0 Hz, 1H), 7.64-7.57 (m, 2H), 7.37-7.26 (m, 2H), 7.07 (d, J=7.6 Hz, 1H), 7.02-6.89 (m, 3H), 5.12 (s, 2H), 4.76-4.66 (m, 2H), 4.63-4.55 (m, 2H), 4.54-4.47 (m, 1H), 4.31 (t, J=7.0 Hz, 3H), 4.21-4.11 (m, 1H), 4.10-3.99 (m, 1H), 3.84-3.74 (m, 0.5H), 3.47-3.42 (m, 0.5H), 3.11-2.96 (m, 5H), 2.27 (t, J=7.5 Hz, 2H), 2.00-1.92 (m, 1H), 1.87-1.76 (m, 3H), 1.64-1.57 (m, 2H), 1.37-1.29 (m, 14H), 1.26-1.11 (m, 5H), 0.91 (s, 9H).

### BF111

### 1. General Procedure for Preparation of methyl 6-[3-[1-[2-[2-(2-prop-2-ynoxyethoxy)ethoxy]acetyl]azetidin-3-yl]oxyphenoxy]pyridine-3-carboxylate

To a solution of methyl 6-[3-(azetidin-3-yloxy)phenoxy]pyridine-3-carboxylate (1 g, 2.41 mmol, 1 eq, TFA) and 2-[2-(2-prop-2-ynoxyethoxy)ethoxy]acetic acid (500 mg, 2.47 mmol, 1.02 eq) in DMF (20 mL) was added DIEA (935.77 mg, 7.24 mmol, 1.26 mL, 3 eq) and HATU (1.84 g, 4.83 mmol, 2 eq) at 0° C., then the reaction was stirred for 1 hr at 25° C. The reaction mixture was diluted with H₂O 20 mL and extracted with EA 60 mL (20 mL*3). The combined organic layers were washed with brine (50 mL*3), dried over anhydrous Na₂SO₄, filtered and concentrated under reduced pressure to give a residue. The residue was purified by column chromatography (SiO₂, Petroleum ether/Ethyl acetate=10/1 to 0/1) to give methyl 6-[3-[1-[2-[2-(2-prop-2-ynoxyethoxy) ethoxy]acetyl]azetidin-3-yl]oxyphenoxy]pyridine-3-carboxylate (1.08 g, 2.23 mmol, 92.36% yield) as a yellow oil.

### Data:

LCMS (ESI⁺): m/z 485.2 (M+1)

### 2. General Procedure for Preparation of methyl 6-[3-[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]azetidin-3-yl]oxyphenoxy]pyridine-3-carboxylate

To a solution of methyl 6-[3-[1-[2-[2-(2-prop-2-ynoxyethoxy)ethoxy]acetyl]azetidin-3-yl]oxyphenoxy]pyridine-3-carboxylate (270.38 mg, 558.06 μmol, 1.2 eq) and 3-[[6-[2-[(3R)-4-(2-azidoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (250 mg, 465.05 μmol, 1 eq) in t-BuOH (3 mL) and H₂O (3 mL) was added sodium; (2R)-2-[(1S)-1,2-dihydroxyethyl]-4-hydroxy-5-oxo-2H-furan-3-olate (92.13 mg, 465.05 μmol, 1 eq) and CuSO₄·5H₂O (58.04 mg, 232.53 μmol, 35.69 μL, 0.5 eq), then the reaction was stirred for 0.5 hr at 50° C. The reaction mixture was diluted with DMF 3 mL, filtered and the filter was purified by reversed-phase HPLC (column: C18 20-35 um 100 A 120 g; mobile phase: [water-ACN]; B %: 0%-55% @ 80 mL/min) to give compound methyl 6-[3-[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]azetidin-3-yl]oxyphenoxy]pyridine-3-carboxylate (270 mg, 264.17 μmol, 56.80% yield) was obtained as a yellow solid.

### Data:

LCMS (ESI⁺): m/z 1044.5 (M+23)

### 3. General Procedure for Preparation of 6-[3-[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]azetidin-3-yl]oxyphenoxy]pyridine-3-carboxylic acid

To a solution of methyl 6-[3-[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]azetidin-3-yl]oxyphenoxy]pyridine-3-carboxylate (270 mg, 264.17 μmol, 1 eq) in THF (1.5 mL) and H₂O (1.5 mL) was added LiOH·H₂O (22.17 mg, 528.34 μmol, 2 eq), then the reaction was stirred for 1 hr at 25° C. THF was removed. 1M FA added to the reaction until pH to 3-4, then the reaction was extracted with DCM 15 mL (5 mL*3). The combined organic layers were dried over anhydrous Na₂SO₄, filtered and concentrated under reduced pressure to give a residue which was purified by reversed-phase HPLC (column: C18 20-35 um 100 A 330 g; mobile phase: [water-ACN]; B %: 0%-60% @ 80 mL/min) to give compound 6-[3-[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]azetidin-3-yl]oxyphenoxy]pyridine-3-carboxylic acid (50 mg, 49.60 μmol, 18.78% yield) as a yellow solid.

### Data:

LCMS (ESI⁺): m/z 1030.5 (M+23)

### 4. General Procedure for Preparation of (2,5-dioxopyrrolidin-1-yl) 6-[3-[1-[2-[2-[2-[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]azetidin-3-yl]oxyphenoxy]pyridine-3-carboxylate

To a mixture of 6-[3-[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]azetidin-3-yl]oxyphenoxy]pyridine-3-carboxylic acid (50 mg, 49.60 μmol, 1 eq) in DMF (1 mL) was added HOSu (8.56 mg, 74.40 μmol, 1.5 eq) and EDCI (19.02 mg, 99.20 μmol, 2 eq), then the mixture was stirred for 0.5 hr at 15° C. The reaction mixture was diluted with H₂O 2 mL and extracted with DCM 9 mL (3 mL*3). The combined organic layers were washed with brine (10 mL*3), dried over anhydrous Na₂SO₄, filtered and concentrated under reduced pressure to give compound (2,5-dioxopyrrolidin-1-yl) 6-[3-[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]azetidin-3-yl]oxyphenoxy]pyridine-3-carboxylate (55 mg, crude) as a yellow oil, which was used to next step without purification.

### Data:

LCMS (ESI⁺): m/z 1105.1 (M+1)

### 5. General Procedure for Preparation of (2S)-2-[[6-[3-[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]azetidin-3-yl]oxyphenoxy]pyridine-3-carbonyl]amino]-5,5-dimethyl-hexanoic acid

To a solution of (2,5-dioxopyrrolidin-1-yl) 6-[3-[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]azetidin-3-yl]oxyphenoxy]pyridine-3-carboxylate (55 mg, 49.77 μmol, 5.31e-1 eq) and (2S)-2-amino-5,5-dimethyl-hexanoic acid (14.92 mg, 93.71 umol, 1 eq) in DMF (0.6 mL), DCM (0.1 mL) and H₂O (0.3 mL) was added DIEA (24.22 mg, 187.41 umol, 32.64 uL, 2 eq). The reaction was degassed and purged with N₂ for 3 times, and then stirred at 20° C. for 1 hrs under N₂ atmosphere. THF was removed. 1M FA added to the reaction until pH to 6-7. Then the mixture was purified by prep-HPLC (FA condition; column: Waters Xbridge Prep OBD C18 150*40 mm*10 um; mobile phase: [H₂O (10 mM NH₄HCO₃)-ACN]; gradient: 20%-50% B over 8.0 min) to give compound (2S)-2-[[6-[3-[[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]azetidin-3-yl]oxyphenoxy]pyridine-3-carbonyl]amino]-5,5-dimethyl-hexanoic acid (29 mg, 25.23 μmol, 26.93% yield, 100% purity) as a yellow solid.

### Data:

LCMS (ESI⁺): m/z 1149.2 (M+1)

¹H NMR (400 MHz, METHANOL-d4) δ=8.95 (s, 2H), 8.65-8.58 (m, 2H), 8.23 (dd, J=2.4, 8.7 Hz, 1H), 8.03 (dd, J=1.3, 8.1 Hz, 1H), 7.95 (d, J=7.9 Hz, 2H), 7.62 (dd, J=4.6, 8.2 Hz, 1H), 7.40-7.28 (m, 2H), 7.00 (d, J=8.7 Hz, 1H), 6.78 (dd, J=1.8, 8.0 Hz, 1H), 6.73 (dd, J=2.0, 8.3 Hz, 1H), 6.69-6.63 (m, 1H), 5.64-5.48 (m, 1H), 5.43-5.31 (m, 1H), 5.12 (s, 2H), 5.00 (br dd, J=3.8, 6.0 Hz, 1H), 4.80-4.67 (m, 3H), 4.63 (s, 3H), 4.49 (dd, J=5.1, 8.5 Hz, 1H), 4.41 (dd, J=6.5, 11.0 Hz, 1H), 4.32 (br dd, J=3.5, 10.6 Hz, 1.5H), 4.11 (s, 2H), 3.98 (dd, J=3.6, 10.6 Hz, 1H), 3.88-3.81 (m, 0.5H), 3.68-3.51 (m, 9H), 3.42-3.34 (m, 1H), 3.15-3.03 (m, 1H), 2.00-1.91 (m, 1H), 1.86-1.74 (m, 1H), 1.40-1.32 (m, 8H), 1.30-1.14 (m, 3H), 0.90 (s, 9H).

### BF112

### 1. General Procedure for Preparation of methyl 6-[3-[(1-tert-butoxycarbonyl-4-piperidyl)oxy]phenoxy]pyridine-3-carboxylate

To a solution of tert-butyl 4-(p-tolylsulfonyloxy) piperidine-1-carboxylate (869.67 mg, 2.45 mmol, 1 eq) and methyl 6-(3-hydroxyphenoxy)pyridine-3-carboxylate (0.6 g, 2.45 mmol, 1 eq) in DMF (30 mL) was added Cs₂CO₃ (1.59 g, 4.89 mmol, 2 eq). The mixture was stirred at 80° C. for 1 hr. The mixture was diluted with water 10 mL, and then extracted with Ethyl acetate 60 mL (20 mL*3). The combined organic layers were washed with sat. NaCl 30 mL, dried over Na₂SO₄, filtered and concentrated under reduced pressure to give a residue which was purified by column chromatography (SiO₂, Petroleum ether/Ethyl acetate=1/0 to 3/1) to give compound methyl 6-[3-[(1-tert-butoxycarbonyl-4-piperidyl)oxy]phenoxy]pyridine-3-carboxylate (1.9 g, 4.43 mmol, 90.62% yield) as a white solid.

### Data:

LCMS (ESI⁺): m/z 429.0 (M+H)

### 2. General Procedure for Preparation of methyl 6-[3-(4-piperidyloxy)phenoxy]pyridine-3-carboxylate

A mixture of methyl 6-[3-[(1-tert-butoxycarbonyl-4-piperidyl)oxy]phenoxy]pyridine-3-carboxylate (1.9 g, 4.43 mmol, 1 eq) in TFA (4 mL) and DCM (20 mL) was stirred at 20° C. for 1 hr. The DCM and TFA was removed under reduced pressure to give compound methyl 6-[3-(4-piperidyloxy)phenoxy]pyridine-3-carboxylate (1.9 g, 4.29 mmol, 96.86% yield, TFA) as a yellow oil.

### Data:

LCMS (ESI⁺): m/z 329.0 (M+H)

### 3. General Procedure for Preparation of methyl 6-[3-[[1-[2-[2-(2-prop-2-ynoxyethoxy)ethoxy]acetyl]-4-piperidyl]oxy]phenoxy]pyridine-3-carboxylate

To a solution of methyl 6-[3-(4-piperidyloxy)phenoxy]pyridine-3-carboxylate (1.9 g, 4.29 mmol, 1 eq, TFA) and 2-[2-(2-prop-2-ynoxyethoxy)ethoxy]acetic acid (868.45 mg, 4.29 mmol, 1 eq) in DMF (0.5 mL) was added HATU (2.45 g, 6.44 mmol, 1.5 eq) and DIEA (1.11 g, 8.59 mmol, 1.50 mL, 2 eq) at 0° C. The mixture was stirred at 20° C. for 1 hr. The mixture was diluted with water 30 mL, and then extracted with Ethyl acetate 120 mL (40 mL*3). The combined organic layers were washed with sat·NaCl 80 mL, dried over Na₂SO₄, filtered and concentrated under reduced pressure to give a residue which was purified by column chromatography (SiO₂, Petroleum ether/Ethyl acetate=1/0 to 0/1) to give compound methyl 6-[3-[[1-[2-[2-(2-prop-2-ynoxyethoxy)ethoxy]acetyl]-4-piperidyl]oxy]phenoxy]pyridine-3-carboxylate (2 g, 3.90 mmol, 90.85% yield) as a yellow oil.

### Data:

LCMS (ESI⁺): m/z 513.0 (M+H)

### 4. General Procedure for Preparation of methyl 6-[3-[[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]-4-piperidyl]oxy]phenoxy]pyridine-3-carboxylate

To a solution of methyl 6-[3-[[1-[2-[2-(2-prop-2-ynoxyethoxy)ethoxy]acetyl]-4-piperidyl]oxy]phenoxy]pyridine-3-carboxylate (286.03 mg, 558.06 μmol, 1.2 eq) and 3-[[6-[2-[(3R)-4-(2-azidoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (250 mg, 465.05 μmol, 1 eq) in t-BuOH (2.5 mL) and H₂O (2.5 mL) was added copper; sulfate (37.11 mg, 232.53 μmol, 35.69 μL, 0.5 eq) and sodium; (2R)-2-[(1S)-1,2-dihydroxyethyl]-4-hydroxy-5-oxo-2H-furan-3-olate (92.13 mg, 465.05 μmol, 1 eq). The mixture was stirred at 50° C. for 1 hr. The mixture was diluted with water 5 mL, and then extracted with Ethyl acetate 15 mL (5 mL*3). The combined organic layers were washed with sat·NaCl 10 mL, dried over Na₂SO₄, filtered and concentrated under reduced pressure to give a residue which was purified by reversed-phase HPLC (column: C18 20-35 um 100 A 120 g; mobile phase: [water-ACN]; B %: 0%-55% @ 100 mL/min) to give compound methyl 6-[3-[[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]-4-piperidyl]oxy]phenoxy]pyridine-3-carboxylate (300 mg, 285.68 μmol, 61.43% yield) as a yellow solid.

### Data:

LCMS (ESI⁺): m/z 1072.4 (M+H)

### 5. General Procedure for Preparation of 6-[3-[[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]-4-piperidyl]oxy]phenoxy]pyridine-3-carboxylic acid

To a solution of methyl 6-[3-[[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]-4-piperidyl]oxy]phenoxy]pyridine-3-carboxylate (300 mg, 285.68 μmol, 1 eq) in THF (1.5 mL) and H₂O (1.5 mL) was added LiOH·H₂O (23.98 mg, 571.36 μmol, 2 eq). The mixture was stirred at 20° C. for 1 hr. The mixture was acid with HCl (1M) to pH=2. The mixture was diluted with water 5 mL, and then extracted with Ethyl acetate 15 mL (5 mL*3). The combined organic layers were washed with sat·NaCl 10 mL, dried over Na₂SO₄, filtered and concentrated under reduced pressure to give compound 6-[3-[[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]-4-piperidyl]oxy]phenoxy]pyridine-3-carboxylic acid (280 mg, 261.06 μmol, 91.38% yield, HCl) as a yellow oil.

### Data:

LCMS (ESI⁺): m/z 1036.1 (M+H)

### 6. General Procedure for Preparation of (2,5-dioxopyrrolidin-1-yl) 6-[3-[[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]-4-piperidyl]oxy]phenoxy]pyridine-3-carboxylate

To a solution of 6-[3-[[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]-4-piperidyl]oxy]phenoxy]pyridine-3-carboxylic acid (260 mg, 242.41 μmol, 1 eq, HCl), HOSU (41.85 mg, 363.62 μmol, 1.5 eq) in DMF (3 mL) was added EDCI (92.94 mg, 484.82 μmol, 2 eq) at 0° C. The mixture was stirred at 20° C. for 0.5 hr. The mixture was diluted with water 5 mL, and then extracted with Ethyl acetate 15 mL (5 mL*3). The combined organic layers were washed with brine 5 mL*2, dried over Na₂SO₄, filtered and concentrated under reduced pressure to give a residue to give compound (2,5-dioxopyrrolidin-1-yl) 6-[3-[[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]-4-piperidyl]oxy]phenoxy]pyridine-3-carboxylate (275 mg, crude) as a light yellow oil.

### Data:

LCMS (ESI⁺): m/z 567.6 (M/2+H)

### 7. General Procedure for Preparation of (2S)-2-[[6-[3-[[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]-4-piperidyl]oxy]phenoxy]pyridine-3-carbonyl]amino]-5,5-dimethyl-hexanoic acid

To a solution of (2,5-dioxopyrrolidin-1-yl) 6-[3-[[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]-4-piperidyl]oxy]phenoxy]pyridine-3-carboxylate (275 mg, 242.68 μmol, 1 eq) and (2S)-2-amino-5,5-dimethyl-hexanoic acid (38.64 mg, 242.68 μmol, 1 eq) in DMF (1.8 mL), DCM (0.3 mL) and H₂O (0.9 mL) was added DIEA (62.73 mg, 485.36 μmol, 84.54 μL, 2 eq) at 0° C. The mixture was stirred at 20° C. for 0.5 hr. The residue was adjusted with FA to pH=7. The mixture was filtered and the filtrate was purified by prep-HPLC (neutral condition; column: Waters Xbridge Prep OBD C18 150*40 mm*10 um; mobile phase: [H₂O (10 mM NH₄HCO₃)-ACN]; gradient: 25%-55% B over 8.0 min) to give compound (2S)-2-[[6-[3-[[1-[2-[2-[2-[[1-[2-[(2R)-4-[5-[1-[(2-cyano-3-pyridyl)methyl]-2,2-dimethyl-3-oxo- pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]-4-piperidyl]oxy]phenoxy]pyridine-3-carbonyl]amino]-5,5-dimethyl-hexanoic acid (100 mg, 84.94 μmol, 35.00% yield, 100% purity) as a yellow solid.

### Data:

LCMS (ESI⁺): m/z 1177.8 (M+H)

¹H NMR (400 MHz, METHANOL-d4) δ ppm 8.95 (s, 2H) 8.60-8.65 (m, 2H) 8.23 (dd, J=8.64, 2.44 Hz, 1H) 8.03 (d, J=8.11 Hz, 1H) 7.92-7.99 (m, 2H) 7.61 (dd, J=8.17, 4.71 Hz, 1H) 7.28-7.36 (m, 2H) 6.98 (d, J=8.70 Hz, 1H) 6.87 (dd, J=8.28, 2.09 Hz, 1H) 6.81 (s, 1H) 6.72 (dd, J=8.11, 1.67 Hz, 1H) 5.32-5.68 (m, 2H) 5.12 (s, 2H) 4.56-4.80 (m, 4H) 4.49 (dd, J=8.40, 5.07 Hz, 1H) 4.32-4.37 (m, 0.5H) 4.25 (d, J=2.38 Hz, 2H) 3.77-3.88 (m, 1.5H) 3.61-3.75 (m, 9H) 3.46-3.59 (m, 2H) 3.35-3.45 (m, 2H) 3.03-3.17 (m, 1H) 1.90-2.05 (m, 3H) 1.67-1.86 (m, 3H) 1.31-1.39 (m, 8H) 1.12-1.31 (m, 3H) 0.90 (s, 9H)

### BF113

### 1. General Procedure for Preparation of methyl 6-[3-[2-(2-bromoethoxy)ethoxy]phenoxy]pyridine-3-carboxylate

To a solution of methyl 6-(3-hydroxyphenoxy)pyridine-3-carboxylate (500 mg, 2.04 mmol, 1 eq) and 1-bromo-2-(2-bromoethoxy) ethane (472.85 mg, 2.04 mmol, 256.29 μL, 1 eq) in DMF (8 mL) was added K₂CO₃ (563.57 mg, 4.08 mmol, 2 eq). The mixture was stirred at 80° C. for 12 hr. The mixture was diluted with water 10 mL, and then extracted with Ethyl acetate 30 mL (10 mL*3). The combined organic layers were washed with sat·NaCl 15 mL, dried over Na₂SO₄, filtered and concentrated under reduced pressure to give a residue. The residue was purified by column chromatography (SiO₂, Petroleum ether/Ethyl acetate=1/0 to 3/1) to give compound methyl 6-[3-[2-(2-bromoethoxy)ethoxy]phenoxy]pyridine-3-carboxylate (410 mg, 1.03 mmol, 50.75% yield) as a light yellow oil.

### Data:

LCMS (ESI⁺): m/z 658.3 (M+H)

### 2. General Procedure for Preparation of methyl 6-[3-[2-[2-[4-[2-(2-prop-2-ynoxyethoxy)ethyl]piperazin-1-yl]ethoxy]ethoxy]phenoxy]pyridine-3-carboxylate

To a solution of 1-[2-(2-prop-2-ynoxyethoxy)ethyl]piperazine (130 mg, 398.39 μmol, 1 eq, TFA) and methyl 6-[3-[2-(2-bromoethoxy)ethoxy]phenoxy]pyridine-3-carboxylate (157.86 mg, 398.39 μmol, 1 eq) in ACN (4 mL) was added K₂CO₃ (110.12 mg, 796.79 μmol, 2 eq). The mixture was stirred at 60° C. for 12 hr. The mixture was diluted with water 5 mL, and then extracted with EtOAc 15 mL (5 mL*3). The combined organic layers were washed with sat. NaCl 10 mL, dried over Na₂SO₄, filtered and concentrated under reduced. The crude product was purified by reversed-phase HPLC (column: C18 20-35 um 100 A 80 g; mobile phase: [water-ACN]; B %: 0%-50% @ 80 mL/min) to give compound methyl 6-[3-[2-[2-[4-[2-(2-prop-2-ynoxyethoxy)ethyl]piperazin-1-yl]ethoxy]ethoxy]phenoxy]pyridine-3-carboxylate (180 mg, 341.16 μmol, 85.63% yield) as a yellow oil.

### Data:

LCMS (ESI⁺): m/z 528.1 (M+H)

### 3. General Procedure for Preparation of methyl 6-[3-[2-[2-[4-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethyl]piperazin-1-yl]ethoxy]ethoxy]phenoxy]pyridine-3-carboxylate

To a solution of methyl 6-[3-[2-[2-[4-[2-(2-prop-2-ynoxyethoxy)ethyl]piperazin-1-yl]ethoxy]ethoxy]phenoxy]pyridine-3-carboxylate (180 mg, 341.16 μmol, 1 eq) and 3-[[6-[2-[(3R)-4-(2-azidoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (183.40 mg, 341.16 μmol, 1 eq) in t-BuOH (2 mL) and H₂O (2 mL) was added copper; sulfate (27.23 mg, 170.58 μmol, 26.18 μL, 0.5 eq) and sodium; (2R)-2-[(1S)-1,2-dihydroxyethyl]-4-hydroxy-5-oxo-2H-furan-3-olate (67.59 mg, 341.16 μmol, 1 eq). The mixture was stirred at 50° C. for 0.5 hr. The mixture was filtered and the filtrate was purified by reversed-phase HPLC (column: C18 20-35 um 100 A 120 g; mobile phase: [water-ACN]; B %: 0%-45%@ 100 mL/min) to give compound methyl 6-[3-[2-[2-[4-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethyl]piperazin-1-yl]ethoxy]ethoxy]phenoxy]pyridine-3-carboxylate (170 mg, 159.60 μmol, 46.78% yield) as a yellow oil

### Data:

LCMS (ESI⁺): m/z 1065.4 (M+H)

### 4. General Procedure for Preparation of 6-[3-[2-[2-[4-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethyl]piperazin-1-yl]ethoxy]ethoxy]phenoxy]pyridine-3-carboxylic acid

To a solution of methyl 6-[3-[2-[2-[4-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethyl]piperazin-1-yl]ethoxy]ethoxy]phenoxy]pyridine-3-carboxylate (170 mg, 159.60 μmol, 1 eq) in THF (1 mL) and H₂O (1 mL) was added LiOH·H₂O (13.39 mg, 319.19 μmol, 2 eq). The mixture was stirred at 15° C. for 0.5 hr. The mixture was acid with HCl (1M) to pH=2. The mixture was diluted with water 5 mL, and then extracted with Ethyl acetate 15 mL (5 mL*3). The combined organic layers were washed with sat·NaCl 10 mL, dried over Na₂SO₄, filtered and concentrated under reduced pressure to give compound 6-[3-[2-[2-[4-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethyl]piperazin-1-yl]ethoxy]ethoxy]phenoxy]pyridine-3-carboxylic acid (130 mg, crude, HCl) as a yellow oil.

### Data:

LCMS (ESI⁺): m/z 1051.1 (M+H)

### 5. General Procedure for Preparation of (2,5-dioxopyrrolidin-1-yl) 6-[3-[2-[2-[4-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethyl]piperazin-1-yl]ethoxy]ethoxy]phenoxy]pyridine-3-carboxylate

To a solution of 6-[3-[2-[2-[4-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethyl]piperazin-1-yl]ethoxy]ethoxy]phenoxy]pyridine-3-carboxylic acid (130 mg, 119.53 μmol, 1 eq, HCl), 1-hydroxypyrrolidine-2,5-dione (20.63 mg, 179.29 μmol, 1.5 eq) in DMF (2 mL) was added EDCI (45.83 mg, 239.05 μmol, 2 eq) at 0° C. The mixture was stirred at 15° C. for 0.5 hr. The mixture was diluted with water 5 mL, and then extracted with DCM 15 mL (5 mL*3). The combined organic layers were washed with sat·NaCl 10 mL, dried over Na₂SO₄, filtered and concentrated under reduced pressure to give compound (2,5-dioxopyrrolidin-1-yl) 6-[3-[2-[2-[4-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethyl]piperazin-1-yl]ethoxy]ethoxy]phenoxy]pyridine-3-carboxylate (130 mg, crude) as a light yellow oil.

### Data:

LCMS (ESI⁺): m/z 1148.1 (M+H)

### 6. General Procedure for Preparation of (2S)-2-[[6-[3-[2-[2-[4-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethyl]piperazin-1-yl]ethoxy]ethoxy]phenoxy]pyridine-3-carbonyl]amino]-5,5-dimethyl-hexanoic acid

To a solution of (2,5-dioxopyrrolidin-1-yl) 6-[3-[2-[2-[4-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethyl]piperazin-1-yl]ethoxy]ethoxy]phenoxy]pyridine-3-carboxylate (130 mg, 113.22 μmol, 1 eq) and (2S)-2-amino-5,5-dimethyl-hexanoic acid (18.03 mg, 113.22 μmol, 1 eq) in DMF (1.8 mL), DCM (0.3 mL) and H₂O (0.9 mL) was added DIEA (29.27 mg, 226.44 μmol, 39.44 μL, 2 eq) at 0° C. The mixture was stirred at 15° C. for 0.5 hr. The mixture was filtered and the filtrate was purified by prep-HPLC (neutral condition; column: Waters Xbridge Prep OBD C18 150*40 mm*10 um; mobile phase: [H₂O (10 mM NH₄HCO₃)-ACN]; gradient: 30%-60% B over 8.0 min) to give compound (2S)-2-[[6-[3-[2-[2-[4-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethyl]piperazin-1-yl]ethoxy]ethoxy]phenoxy]pyridine-3-carbonyl]amino]-5,5-dimethyl-hexanoic acid (30 mg, 24.77 μmol, 21.87% yield, 98.431% purity) as a yellow solid.

### Data:

LCMS (ESI⁺): m/z 1192.3 (M+H)

¹H NMR (400 MHz, METHANOL-d4) δ ppm 8.94 (s, 2H) 8.59-8.65 (m, 2H) 8.24 (dd, J=8.64, 2.44 Hz, 1H) 8.01-8.05 (m, 1H) 7.92-8.00 (m, 2H) 7.61 (dd, J=8.11, 4.65 Hz, 1H) 7.27-7.35 (m, 2H) 6.97 (d, J=8.70 Hz, 1H) 6.80-6.85 (m, 1H) 6.69-6.78 (m, 2H) 5.35-5.76 (m, 2H) 5.11 (s, 2H) 4.68-4.77 (m, 2H) 4.64 (s, 3H) 4.55-4.61 (m, 1H) 4.45 (dd, J=7.39, 4.89 Hz, 1H) 4.32-4.40 (m, 1H) 4.11-4.19 (m, 2H) 3.85-3.92 (m, 1H) 3.79-3.84 (m, 2H) 3.67-3.77 (m, 6H) 3.63-3.66 (m, 2H) 3.52-3.62 (m, 1H) 3.35-3.43 (m, 1H) 2.82-3.05 (m, 11H) 1.91-2.02 (m, 1H) 1.73-1.84 (m, 1H) 1.37 (s, 6H) 1.28-1.33 (m, 3.5H) 1.16 (br d, J=6.68 Hz, 1.5H) 0.88 (s, 9H).

### BF114

### 1. General Procedure for Preparation of methyl 6-[3-[2-(2-oxoethoxy)ethoxy]phenoxy]pyridine-3-carboxylate

To a solution of methyl 6-[3-[2-(2-hydroxyethoxy)ethoxy]phenoxy]pyridine-3-carboxylate (800 mg, 2.40 mmol, 1 eq) in DCM (65 mL) was added NaHCO₃ (705.68 mg, 8.40 mmol, 326.86 μL, 3.5 eq). Then DMP (2.54 g, 6.00 mmol, 1.86 mL, 2.5 eq) was added at 0° C., then the reaction was stirred for 1 hr at 15° C. To the reaction was added saturated NaHCO₃ solution (65 mL) and saturated Na₂S₂O₃ solution (65 mL), then the mixture was stirred for 0.5 hr and extracted with DCM (100 mL*3). The combined organic layers were dried over anhydrous Na₂SO₄, filtered and concentrated under reduced pressure to give a residue. The residue was purified by column chromatography (SiO₂, Petroleum ether/Ethyl acetate=10/1 to 3/1) to give compound methyl 6-[3-[2-(2-oxoethoxy)ethoxy]phenoxy]pyridine-3-carboxylate (615 mg, 1.86 mmol, 77.34% yield) as a colorless oil.

### 2. General Procedure for Preparation of methyl 6-[3-[2-[2-[3-[2-(2-prop-2-ynoxyethoxy)ethoxy]azetidin-1-yl]ethoxy]ethoxy]phenoxy]pyridine-3-carboxylate

TEA (161.51 mg, 1.60 mmol, 222.15 μL, 1 eq) was added to a solution of 3-[2-(2-prop-2-ynoxyethoxy)ethoxy]azetidine (500 mg, 1.60 mmol, 1 eq, TFA) in MeOH (10 mL) until pH=8˜9, then the reaction was stirred for 10 min at 20° C., then methyl 6-[3-[2-(2-oxoethoxy)ethoxy]phenoxy]pyridine-3-carboxylate (615 mg, 1.86 mmol, 1.16 eq) was added, AcOH (95.84 mg, 1.60 mmol, 91.37 μL, 1 eq) was added to adjust pH <5, then the reaction was stirred for 10 min at 20° C., NaBH3CN (200.59 mg, 3.19 mmol, 2 eq) were added to the reaction, then the reaction was stirred for 1 hr at 20° C. The mixture was filtered and the filtrate was purified by reversed-phase HPLC (column: C18 20-35 um 100 A 80 g; mobile phase: [water-ACN]; B %: 0%-20% @ 70 mL/min) to give compound methyl 6-[3-[2-[2-[3-[2-(2-prop-2-ynoxyethoxy)ethoxy]azetidin-1-yl]ethoxy]ethoxy]phenoxy]pyridine-3-carboxylate (250 mg, 485.85 μmol, 30.44% yield) as colorless oil.

### Data:

LCMS (ESI⁺): m/z 515.3 (M+H)

### 3. General Procedure for Preparation of methyl 6-[3-[2-[2-[3-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]azetidin-1-yl]ethoxy]ethoxy]phenoxy]pyridine-3-carboxylate

To a solution of methyl 6-[3-[2-[2-[3-[2-(2-prop-2-ynoxyethoxy)ethoxy]azetidin-1-yl]ethoxy]ethoxy]phenoxy]pyridine-3-carboxylate (110 mg, 213.77 μmol, 1 eq) and 3-[[6-[2-[(3R)-4-(2-azidoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (149.39 mg, 277.90 μmol, 1.3 eq) in t-BuOH (1 mL) and H₂O (1 mL) was added sodium; (2R)-2-[(1S)-1,2-dihydroxyethyl]-4-hydroxy-5-oxo-2H-furan-3-olate (42.35 mg, 213.77 μmol, 1 eq) and copper; sulfate (17.06 mg, 106.89 μmol, 16.40 μL, 0.5 eq), then the reaction was stirred for 0.5 hr at 50° C. The reaction mixture was diluted with DMF 3 mL, filtered and the filtrate was purified by reversed-phase HPLC (column: C18 20-35 um 100 A 120 g; mobile phase: [water-ACN]; B %: 0%-60% @ 80 mL/min) to give compound methyl 6-[3-[2-[2-[3-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]azetidin-1-yl]ethoxy]ethoxy]phenoxy]pyridine-3-carboxylate (150 mg, 142.57 μmol, 33.35% yield) as a yellow solid

### Data:

LCMS (ESI⁺): m/z 1052.6 (M+H)

### 4. General Procedure for Preparation of 6-[3-[2-[2-[3-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]azetidin-1-yl]ethoxy]ethoxy]phenoxy]pyridine-3-carboxylic acid

To a solution of methyl 6-[3-[2-[2-[3-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]azetidin-1-yl]ethoxy]ethoxy]phenoxy]pyridine-3-carboxylate (150 mg, 142.57 μmol, 1 eq) in THF (1 mL) and H₂O (1 mL) was added LiOH·H₂O (11.96 mg, 285.13 μmol, 2 eq) at 0° C., then the reaction was stirred for 1 hr at 0° C. THF was removed under reduced pressure. The aqueous layer was adjusted pH to 3-4 with FA. And extracted with DCM 15 mL (5 mL*3). The combined organic layers were dried over anhydrous Na₂SO₄, filtered and concentrated under reduced pressure to give compound 6-[3-[2-[2-[3-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]azetidin-1-yl]ethoxy]ethoxy]phenoxy]pyridine-3-carboxylic acid (150 mg, crude, HCl) as a yellow solid.

### Data:

LCMS (ESI⁺): m/z 1038.1 (M+H)

### 5. General Procedure for Preparation of (2,5-dioxopyrrolidin-1-yl) 6-[3-[2-[2-[3-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]azetidin-1-yl]ethoxy]ethoxy]phenoxy]pyridine-3-carboxylate

To a mixture of 6-[3-[2-[2-[3-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]azetidin-1-yl]ethoxy]ethoxy]phenoxy]pyridine-3-carboxylic acid (130 mg, 120.98 μmol, 1 eq, HCl) in DMF (2 mL) was added HOSu (20.88 mg, 181.47 μmol, 1.5 eq) and EDCI (46.38 mg, 241.96 μmol, 2 eq), then the mixture was stirred for 0.5 hr at 15° C. The reaction mixture was diluted with H₂O 2 mL and extracted with DCM 9 mL (3 mL*3). The combined organic layers were washed with brine (10 mL*3), dried over anhydrous Na₂SO₄, filtered and concentrated under reduced pressure to give compound (2,5-dioxopyrrolidin-1-yl) 6-[3-[2-[2-[3-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]azetidin-1-yl]ethoxy]ethoxy]phenoxy]pyridine-3-carboxylate (137 mg, crude) as a yellow oil.

### 6. General Procedure for Preparation of (2S)-2-[[6-[3-[2-[2-[3-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]azetidin-1-yl]ethoxy]ethoxy]phenoxy]pyridine-3-carbonyl]amino]-5,5-dimethyl-hexanoic acid

To a solution of (2,5-dioxopyrrolidin-1-yl) 6-[3-[2-[2-[3-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]azetidin-1-yl]ethoxy]ethoxy]phenoxy]pyridine-3-carboxylate (137 mg, 120.69 μmol, 1.29 eq) and (2S)-2-amino-5,5-dimethyl-hexanoic acid (14.92 mg, 93.71 umol, 1 eq) in DMF (1.2 mL), DCM (0.2 mL) and H₂O (0.6 mL) was added DIEA (24.22 mg, 187.41 μmol, 32.64 uL, 2 eq). The reaction was degassed and purged with N₂ for 3 times, and then stirred at 20° C. for 1 hr under N₂ atmosphere. DCM was removed under reduced pressure. The aqueous layer was adjusted pH to 6-7 with 1 M aq·FA. The mixture was purified by prep-HPLC (neutral condition; column: Waters Xbridge Prep OBD C18 150*40 mm*10 um; mobile phase: [H₂O (10 mM NH₄HCO₃)-ACN]; gradient: 20%-55% B over 8.0 min) to give compound (2S)-2-[[6-[3-[2-[2-[3-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]azetidin-1-yl]ethoxy]ethoxy]phenoxy]pyridine-3-carbonyl]amino]-5,5-dimethyl-hexanoic acid (46 mg, 39.01 μmol, 41.62% yield, 100% purity) as a yellow solid.

### Data:

LCMS (ESI⁺): m/z 1179.3 (M+H)

¹H NMR (400 MHz, METHANOL-d₄) δ=8.95 (s, 2H), 8.66-8.59 (m, 2H), 8.24 (dd, J=2.4, 8.7 Hz, 1H), 8.03 (dd, J=1.3, 8.3 Hz, 1H), 8.00-7.93 (m, 2H), 7.62 (dd, J=4.7, 8.1 Hz, 1H), 7.37-7.28 (m, 2H), 6.97 (d, J=8.7 Hz, 1H), 6.83 (dd, J=1.9, 8.3 Hz, 1H), 6.77-6.70 (m, 2H), 5.71-5.54 (m, 1H), 5.49-5.36 (m, 1H), 5.11 (s, 2H), 4.81-4.68 (m, 2H), 4.63 (s, 3H), 4.45 (dd, J=5.0, 7.3 Hz, 1H), 4.42-4.25 (m, 4H), 4.16-4.11 (m, 2H), 3.98-3.89 (m, 2H), 3.87-3.77 (m, 2.5H), 3.75-3.70 (m, 2H), 3.69-3.65 (m, 2H), 3.65-3.51 (m, 7H), 3.50-3.47 (m, 0.5H), 3.41-3.34 (m, 2H), 3.15-3.04 (m, 1H), 2.02-1.91 (m, 1H), 1.83-1.73 (m, 1H), 1.37 (s, 6H), 1.33-1.29 (m, 3.5H), 1.18-1.13 (m, 1.5H), 0.88 (s, 9H)

### BF119

### 1. General Procedure for Preparation of 3-(2-prop-2-ynoxyethoxy)propanenitrile

To a solution of 2-prop-2-ynoxyethanol (10 g, 99.88 mmol, 1 eq) in prop-2-enenitrile (53.00 g, 998.85 mmol, 66.25 mL, 10 eq) was added DBU (7.60 g, 49.94 mmol, 7.53 mL, 0.5 eq) at 0° C., then the reaction was stirred for 30 min, then the mixture was stirred at 70° C. for 11.5 hr. The reaction solution was adjusted to pH=7 with 6N HCl. The reaction mixture was partitioned between water 100 mL and Ethyl acetate 100 mL. The organic phase was separated, washed with brine 150 mL (50 mL*3), dried over [Na₂SO₄], filtered and concentrated under reduced pressure to give a residue. The residue was purified by flash silica gel chromatography (ISCO®; 80 g SepaFlash® Silica Flash Column, Eluent of 0˜40% Ethyl acetate/Petroleum ether gradient @ 90 mL/min) to give compound 3-(2-prop-2-ynoxyethoxy)propanenitrile (15 g, 97.93 mmol, 98.04% yield) as a white solid.

### Data:

¹H NMR (400 MHz, CHLOROFORM-d) δ=4.20 (d, J=2.3 Hz, 2H), 3.76-3.66 (m, 6H), 2.63 (t, J=6.5 Hz, 2H), 2.45 (t, J=2.4 Hz, 1H)

### 2. General Procedure for Preparation of 3-(2-prop-2-ynoxyethoxy) propan-1-amine

To a solution of tris(2,3,4,5,6-pentafluorophenyl) borane (1.67 g, 3.26 mmol, 0.5 eq) in CHCl₃ (50 mL) was added diethylsilane (1.73 g, 19.59 mmol, 3 eq) and 3-(2-prop-2-ynoxyethoxy)propanenitrile (1 g, 6.53 mmol, 1 eq) at 0° C., then the reaction was stirred for 30 min, then the mixture was stirred at 25° C. for 11.5 hr. Filtered and concentrated under reduced pressure to give a residue. The residue was purified by prep-TLC to give compound 3-(2-prop-2-ynoxyethoxy) propan-1-amine (1.5 g, 9.54 mmol, 24.36% yield) as a yellow oil.

### Data:

¹H NMR (400 MHz, CHLOROFORM-d) δ=4.18 (d, J=2.2 Hz, 2H), 3.69-3.63 (m, 2H), 3.62-3.53 (m, 4H), 2.85 (t, J=6.6 Hz, 2H), 2.44 (t, J=2.3 Hz, 1H), 1.78 (quin, J=6.3 Hz, 2H)

### 3. General Procedure for Preparation of 3-(2-prop-2-ynoxyethoxy) propyl cyanate

To a solution of bis(trichloromethyl) carbonate (151.01 mg, 508.87 μmol, 0.4 eq) in DCM (1 mL) was added 3-(2-prop-2-ynoxyethoxy) propan-1-amine (0.2 g, 1.27 mmol, 1 eq) and TEA (257.46 mg, 2.54 mmol, 354.14 μL, 2.00 eq). The mixture was stirred at 0° C. for 0.5 hr. The reaction mixture was quenched by addition Sat. NaHCO₃ 2 mL, and then extracted with Ethyl acetate 3 mL (1 mL*3). The combined organic layers were washed with brine 2 mL (1 mL*2), dried over Na₂SO₄, filtered and concentrated under reduced pressure to give compound 3-(2-prop-2-ynoxyethoxy) propyl cyanate (0.35 g, 1.91 mmol, 75.08% yield) as a yellow oil.

### 4. General Procedure for Preparation of methyl (2S)-5,5-dimethyl-2-[[6-[3-[1-[3-(2-prop-2-ynoxyethoxy) propylcarbamoyl]azetidin-3-yl]oxyphenoxy]pyridine-3-carbonyl]amino]hexanoate

To a stirred solution of methyl (2S)-2-[[6-[3-(azetidin-3-yloxy)phenoxy]pyridine-3-carbonyl]amino]-5,5-dimethyl-hexanoate (0.3 g, 540.01 μmol, 1 eq, TFA) in THF (3 mL) was added TEA (163.93 mg, 1.62 mmol, 225.49 μL, 3 eq) at 0° C., then 3-(2-prop-2-ynoxyethoxy) propyl cyanate (197.87 mg, 1.08 mmol, 2 eq) was added at 0° C., then the reaction was stirred for 0.5 hr at 20° C. The reaction mixture was partitioned between water 3 mL and Ethyl acetate 3 mL. The organic phase was separated, washed with brine 3 mL (1 mL*3), dried over [Na₂SO₄], filtered and concentrated under reduced pressure to give a residue. The residue was purified by prep-TLC to give compound methyl (2S)-5, 5-dimethyl-2-[[6-[3-[1-[3-(2-prop-2-ynoxyethoxy) propylcarbamoyl]azetidin-3-yl]oxyphenoxy]pyridine-3-carbonyl]amino]hexanoate (80 mg, 128.06 μmol, 23.71% yield) as a white solid.

### Data:

LCMS (ESI⁺): m/z 625.32 (M+H)

### 5. General Procedure for Preparation of methyl (2S)-2-[[6-[3-[1-[3-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]propylcarbamoyl]azetidin-3-yl]oxyphenoxy]pyridine-3-carbonyl]amino]-5,5-dimethyl-hexanoate

To a solution of themethyl (2S)-5,5-dimethyl-2-[[6-[3-[1-[3-(2-prop-2-ynoxyethoxy) propylcarbamoyl]azetidin-3-yl]oxyphenoxy]pyridine-3-carbonyl]amino]hexanoate (70 mg, 112.05 μmol, 1 eq) and 3-[[6-[2-[(3R)-4-(2-azidoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (78.31 mg, 145.66 μmol, 1.3 eq) in t-BuOH (0.5 mL) and H₂O (0.5 mL) was added copper; sulfate (17.88 mg, 112.05 μmol, 17.20 μL, 1 eq), sodium; (2R)-2-[(1S)-1,2-dihydroxyethyl]-4-hydroxy-5-oxo-2H-furan-3-olate (22.20 mg, 112.05 μmol, 1 eq). The reaction was stirred for 1 hr at 50° C. The reaction mixture was partitioned between water 2 mL and Ethyl acetate 2 mL. The organic phase was separated, washed with brine 3 mL (1 mL*3), dried over [Na₂SO₄], filtered and concentrated under reduced pressure to give a residue. The residue was purified by prep-TLC to give compound methyl (2S)-2-[[6-[3-[1-[3-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]propylcarbamoyl]azetidin-3-yl]oxyphenoxy]pyridine-3-carbonyl]amino]-5,5-dimethyl-hexanoate (50 mg, 43.02 μmol, 38.39% yield) as a white solid.

### Data:

LCMS (ESI⁺): m/z 1162.6 (M+H)

### 6. General Procedure for Preparation of (2S)-2-[[6-[3-[1-[3-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]propylcarbamoyl]azetidin-3-yl]oxyphenoxy]pyridine-3-carbonyl]amino]-5,5-dimethyl-hexanoic acid

The deprotection of methyl (2S)-2-[[6-[3-[1-[3-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]propylcarbamoyl]azetidin-3-yl]oxyphenoxy]pyridine-3-carbonyl]amino]-5,5-dimethyl-hexanoate (45 mg, 38.72 μmol, 1 eq) was performed analogously to the description above of BF083 to give the compound of the title BF119 (13 mg, 10.75 μmol, 27.77% yield, 98.756% purity, FA) as a yellow solid.

### Data;

LCMS (ESI⁺): m/z 1148.3 (M+H)

¹H NMR (400 MHz, METHANOL-d4) δ=8.95 (s, 2H), 8.64-8.59 (m, 2H), 8.23 (dd, J=2.4, 8.7 Hz, 1H), 8.03 (d, J=8.1 Hz, 1H), 7.99-7.92 (m, 2H), 7.61 (dd, J=4.8, 8.1 Hz, 1H), 7.38-7.27 (m, 2H), 6.99 (d, J=8.6 Hz, 1H), 6.74 (ddd, J=1.8, 8.3, 14.9 Hz, 2H), 6.66 (t, J=2.2 Hz, 1H), 5.69-5.52 (m, 1H), 5.49-5.34 (m, 1H), 5.12 (s, 2H), 4.97 (tdd, J=3.4, 6.6, 9.9 Hz, 2H), 4.80-4.69 (m, 2H), 4.65 (s, 3H), 4.50 (dd, J=5.1, 8.9 Hz, 1H), 4.35 (br d, J=11.5 Hz, 1H), 4.29 (dd, J=6.4, 9.3 Hz, 2H), 3.88 (dd, J=3.8, 9.1 Hz, 2H), 3.70-3.65 (m, 2H), 3.62-3.58 (m, 2H), 3.52 (t, J=6.0 Hz, 2H), 3.42-3.34 (m, 1H), 3.21 (t, J=6.6 Hz, 2H), 3.16-3.00 (m, 1H), 2.01-1.89 (m, 1H), 1.86-1.78 (m, 1H), 1.77-1.69 (m, 2H), 1.36 (s, 8H), 1.31 (br d, J=2.0 Hz, 1.5H), 1.15 (br d, J=6.9 Hz, 1.5H), 0.91 (s, 9H)

### BF121

### 1. General Procedure for Preparation of 2-[2-(4-benzyloxybutoxy)ethoxy]tetrahydropyran

To a solution of 2-(2-bromoethoxy)tetrahydropyran (13.22 g, 63.25 mmol, 9.55 mL) and 4-benzyloxybutan-1-ol (9.5 g, 52.71 mmol, 9.24 mL) in NaOH (250 mL) was added Bu₄NHSO₄ (2.68 g, 7.91 mmol) at 15° C. The mixture was stirred at 70° C. for 4 hr. The mixture was diluted with water 200 mL, and then extracted with Ethyl acetate 600 mL (200 mL*3). The combined organic layers were washed with sat·NaCl 300 mL, dried over Na₂SO₄, filtered and concentrated under reduced pressure to give a residue. The residue was purified by flash silica gel chromatography (ISCO®; 80 g SepaFlash® Silica Flash Column, Eluent of 0˜12% Ethyl acetate/Petroleum ether gradient @ 100 mL/min) to give compound 2-[2-(4-benzyloxybutoxy)ethoxy]tetrahydropyran (9.3 g, 30.15 mmol, 57.21% yield) as a yellow oil.

### 2. General Procedure for Preparation of 4-(2-tetrahydropyran-2-yloxyethoxy)butan-1-ol

To a solution of 2-[2-(4-benzyloxybutoxy)ethoxy]tetrahydropyran (8 g, 25.94 mmol) in THF (200 mL) was added Pd/C (5 g, 10% purity) under N₂. The suspension was degassed under vacuum and purged with H₂ several times. The mixture was stirred under H₂ (15 psi) at 20° C. for 1 hour. The catalyst was removed by filtration through celite, which was then washed with Ethyl acetate (30 ml). The filtrate was concentrated to an oil. The residue was purified by flash silica gel chromatography (ISCO®; 100 g SepaFlash® Silica Flash Column, Eluent of 0˜80% Ethylacetate/Petroleum ether gradient @ 100 mL/min) to give compound 4-(2-tetrahydropyran-2-yloxyethoxy)butan-1-ol (5.2 g, 23.82 mmol, 91.84% yield) as a colorless oil.

### 3. General Procedure for Preparation of 2-[2-(4-prop-2-ynoxybutoxy)ethoxy]tetrahydropyran

To a stirred solution of 4-(2-tetrahydropyran-2-yloxyethoxy)butan-1-ol (5.7 g, 26.11 mmol) in THF (100 mL) was added NaH (2.09 g, 52.22 mmol, 60% purity) at 0° C., then the reaction was stirred for 0.5 hr at 0° C., then 3-bromoprop-1-yne (3.11 g, 26.11 mmol, 2.25 mL) was added at 0° C., then the reaction was stirred for 0.5 hr at 15° C. The mixture was quenched with saturated NH₄Cl solution (100 mL) at 0° C., and then extracted with Ethyl acetate 450 (150 mL*3). The combined organic layers were washed with sat·NaCl 200 mL, dried over Na₂SO₄, filtered and concentrated under reduced pressure to give a residue. The residue was purified by column chromatography (SiO₂, Petroleum ether/Ethyl acetate=1/0 to 10/1) to give compound 2-[2-(4-prop-2-ynoxybutoxy)ethoxy]tetrahydropyran (5 g, 19.51 mmol, 74.70% yield) as a colorless oil.

### 4. General Procedure for Preparation of 4-(2-tetrahydropyran-2-yloxyethoxy)butan-1-ol

To a solution of 2-[2-(4-prop-2-ynoxybutoxy)ethoxy]tetrahydropyran (3.2 g, 12.48 mmol) in MeOH (32 mL) was added 4-methylbenzenesulfonic acid; hydrate (237.46 mg, 1.25 mmol). The mixture was stirred at 15° C. for 12 hr. The mixture was diluted with water 20 mL, and then extracted with Ethyl acetate 120 mL (40 mL*3). The combined organic layers were washed with sat·NaCl 30 mL, dried over Na₂SO₄, filtered and concentrated under reduced pressure to give a residue. The residue was purified by column chromatography (SiO₂, Petroleum ether/Ethyl acetate=1/0 to 10/1) to give compound 2-(4-prop-2-ynoxybutoxy) ethanol (1.45 g, 8.42 mmol, 67.44% yield) as a colorless oil.

### Data:

¹H NMR (400 MHz, CHLOROFORM-d) 0 ppm 4.11-4.14 (m, 2H) 3.76-3.67 (m, 2H)

### 3.58-3.43 (m, 6H) 2.42 (t, J=2.38 Hz, 1H) 2.25 (s, 1H) 1.70-1.62 (m, 4H)

### 5. General Procedure for Preparation of 1-(2-iodoethoxy)-4-prop-2-ynoxy-butane

A mixture of 2-(4-prop-2-ynoxybutoxy) ethanol (500 mg, 2.90 mmol), PPh₃ (913.78 mg, 3.48 mmol), imidazole (237.17 mg, 3.48 mmol) in DCM (8 mL) was degassed and purged with N₂ for 3 times, and then the mixture was stirred at 0° C. for 30 min under N₂ atmosphere. Then 12 (884.24 mg, 3.48 mmol, 701.78 μL) was added to the mixture at 0° C. The mixture was stirred at 25° C. for 2 h. The mixture was diluted with water 8 mL, and then extracted with DCM 30 mL (10 mL*3). The combined organic layers were washed with sat·NaCl 10 mL, dried over Na₂SO₄, filtered and concentrated under reduced pressure to give a residue. The residue was purified by prep-TLC (SiO₂, Petroleum ether: Ethyl acetate=3:1) to give compound 1-(2-iodoethoxy)-4-prop-2-ynoxy-butane (720 mg, 2.55 mmol, 87.91% yield) as a yellow oil.

### Data:

¹H NMR (400 MHz, CHLOROFORM-d) 0 ppm 4.13 (d, J=2.38 Hz, 2H) 3.68 (t, J=6.75 Hz, 2H) 3.46-3.57 (m, 4H) 3.24 (t, J=6.82 Hz, 2H) 2.42 (t, J=2.38 Hz, 1H) 1.60-1.73 (m, 4H)

### 6. General Procedure for Preparation of 2-(4-prop-2-ynoxybutoxy)ethanesulfonyl chloride

1-(2-iodoethoxy)-4-prop-2-ynoxy-butane (526.16 mg, 1.87 mmol), thiourea (212.95 mg, 2.80 mmol) in EtOH (5 mL) was stirred for 1 hr at 80° C., TLC showed R1 was consumed and two new spots were detected, then the reaction was concentrated to give a residue, then the residue in ACN (1 mL) was added to a solution of NCS (1.25 g, 9.33 mmol) in HCl (2 M, 652.76 μL) and ACN (5 mL) at 0° C., then the reaction was stirred for 0.5 hr at 15° C. The reaction mixture was diluted with H₂O 5 mL and extracted with DCM 15 mL (5 mL*3). The combined organic layers were dried over anhydrous Na₂SO₄, filtered and concentrated under reduced pressure to give compound 2-(4-prop-2-ynoxybutoxy) ethanesulfonyl chloride (475 mg, crude) as a black oil.

### 7. General Procedure for Preparation of methyl (2S)-5,5-dimethyl-2-[[6-[3-[1-[2-(4-prop-2-ynoxybutoxy)ethylsulfonyl]azetidin-3-yl]oxyphenoxy]pyridine-3-carbonyl]amino]hexanoate

To a solution of methyl (2S)-2-[[6-[3-(azetidin-3-yloxy)phenoxy]pyridine-3-carbonyl]amino]-5,5-dimethyl-hexanoate (400 mg, 720.02 μmol, TFA) in DCM (8 mL) was added TEA (218.58 mg, 2.16 mmol, 300.65 μL), then 2-(4-prop-2-ynoxybutoxy)ethanesulfonyl chloride (366.82 mg, 1.44 mmol) in DCM (1 mL) was added at 0° C. and the reaction was stirred for 0.5 hr at 15° C. The reaction mixture was diluted with H₂O 10 mL and extracted with DCM 30 mL (10 mL*3). The combined organic layers were dried over anhydrous Na₂SO₄, filtered and concentrated under reduced pressure to give compound methyl (2S)-5,5-dimethyl-2-[[6-[3-[1-[2-(4-prop-2-ynoxybutoxy)ethylsulfonyl]azetidin-3-yl]oxyphenoxy]pyridine-3-carbonyl]amino]hexanoate (475 mg, crude) as a brown oil.

### Data:

LCMS (ESI⁺): m/z 660.0 (M+H)

### 8. General Procedure for Preparation of: methyl (2S)-2-[[6-[3-[1-[2-[4-[[1-[2-[(2S)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]butoxy]ethylsulfonyl]azetidin-3-yl]oxyphenoxy]pyridine-3-carbonyl]amino]-5,5-dimethyl-hexanoate

To a solution of methyl (2S)-5,5-dimethyl-2-[[6-[3-[1-[2-(4-prop-2-ynoxybutoxy)ethylsulfonyl]azetidin-3-yl]oxyphenoxy]pyridine-3-carbonyl]amino]hexanoate (110 mg, 166.72 μmol) and 3-[[6-[2-[(3R)-4-(2-azidoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (71.70 mg, 133.38 μmol) in t-BuOH (1 mL) and H₂O (1 mL) was added copper; sulfate (13.30 mg, 83.36 μmol, 12.79 μL) and sodium; (2R)-2-[(1S)-1,2-dihydroxyethyl]-4-hydroxy-5-oxo-2H-furan-3-olate (33.03 mg, 166.72 μmol, 1 eq). The mixture was stirred at 50° C. for 1 hr. The mixture was diluted with water 5 mL, and then extracted with Ethyl acetate 15 mL (5 mL*3). The combined organic layers were washed with sat·NaCl 10 mL, dried over Na₂SO₄, filtered and concentrated under reduced pressure to give a residue. The residue was purified by prep-TLC (SiO₂, Petroleum ether: Ethyl acetate=0:1) to give compound methyl (2S)-2-[[6-[3-[1-[2-[4-[[1-[2-[(2S)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]butoxy]ethylsulfonyl]azetidin-3-yl]oxyphenoxy]pyridine-3-carbonyl]amino]-5,5-dimethyl-hexanoate (32 mg, 26.73 μmol, 16.03% yield) as a yellow oil.

### Data:

LCMS (ESI⁺): m/z 1197.6 (M+H)

### 9. General Procedure for Preparation of: (2S)-2-[[6-[3-[1-[2-[4-[[1-[2-[(2S)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]butoxy]ethylsulfonyl]azetidin-3-yl]oxyphenoxy]pyridine-3-carbonyl]amino]-5,5-dimethyl-hexanoic acid

The deprotection of methyl (2S)-2-[[6-[3-[1-[2-[4-[[1-[2-[(2S)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]butoxy]ethylsulfonyl]azetidin-3-yl]oxyphenoxy]pyridine-3-carbonyl]amino]-5,5-dimethyl-hexanoate (32 mg, 26.73 μmol) in THF (0.5 mL) and H₂O (0.5 mL) was performed analogously to the description above of BF083 to give the compound of the title BF121 (14 mg, 11.83 μmol, 44.27% yield, 100% purity) as a yellow solid.

### Data:

LCMS (ESI⁺): m/z 1183.4 (M+H)

¹H NMR (400 MHz, METHANOL-d₄) δ=8.95 (s, 2H), 8.66-8.58 (m, 2H), 8.23 (dd, J=2.4, 8.7 Hz, 1H), 8.06-8.00 (m, 1H), 7.98-7.88 (m, 2H), 7.61 (dd, J=4.8, 8.1 Hz, 1H), 7.40-7.27 (m, 2H), 7.00 (d, J=8.7 Hz, 1H), 6.78 (dd, J=1.8, 8.3 Hz, 1H), 6.72 (dd, J=2.0, 8.4 Hz, 1H), 6.67 (t, J=2.2 Hz, 1H), 5.66-5.49 (m, 1H), 5.46-5.34 (m, 1H), 5.12 (s, 2H), 5.04-4.94 (m, 1H), 4.83-4.68 (m, 4H), 4.68-4.60 (m, 1H), 4.58 (s, 2H), 4.50 (dd, J=5.0, 8.7 Hz, 1H), 4.41-4.27 (m, 3H), 4.04 (dd, J=4.5, 9.2 Hz, 2H), 3.78 (t, J=5.7 Hz, 2H), 3.53-3.45 (m, 4H), 3.38 (t, J=5.7 Hz, 2H), 3.18-3.00 (m, 1H), 2.04-1.89 (m, 1H), 1.88-1.72 (m, 1H), 1.66-1.60 (m, 4H), 1.42-1.32 (m, 8H), 1.32-1.28 (m, 2H), 1.16 (br d, J=6.1 Hz, 2H), 0.91 (s, 9H)

### BF125

### 1. General Procedure for Preparation of benzyl 3-(2-tert-butoxy-2-oxo-ethoxy)pyrrolidine-1-carboxylate

To a solution of benzyl 3-hydroxypyrrolidine-1-carboxylate (1 g, 4.52 mmol) in THF (10 mL) was added NaH (361.54 mg, 9.04 mmol, 60% purity) at 0° C. for 0.5 h. Then tert-butyl 2-bromoacetate (969.75 mg, 4.97 mmol, 734.10 μL) was added to the mixture. The mixture was stirred at 15° C. for 0.5 h. The mixture was quenched with saturated NH₄Cl solution (10 mL) at 0° C. and extracted with EA 30 mL (10 mL*3). The organic layers were washed with sat. NaCl 30 mL, dried over anhydrous Na₂SO₄, filtered and concentrated under reduced pressure to give a residue. The residue was purified by column chromatography (SiO₂, Petroleum ether/Ethyl acetate=1/0 to 2/1) to give compound benzyl 3-(2-tert-butoxy-2-oxo-ethoxy)pyrrolidine-1-carboxylate (1.1 g, 3.28 mmol, 72.56% yield) as off-white liquid.

### Data:

LCMS (ESI⁺): m/z 280.1 (M-56+H)

¹H NMR (400 MHz, CHLOROFORM-d) δ=7.41-7.28 (m, 5H), 5.19-5.09 (m, 2H), 4.22-4.16 (m, 1H), 4.04-3.90 (m, 2H), 3.64-3.47 (m, 4H), 2.16-2.08 (m, 1H), 2.03-1.88 (m, 1H), 1.48 (s, 9H)

### 2. General Procedure for Preparation of t tert-butyl 2-pyrrolidin-3-yloxyacetate

A mixture of benzyl 3-(2-tert-butoxy-2-oxo-ethoxy)pyrrolidine-1-carboxylate (1 g, 2.98 mmol), Pd/C (3.17 g, 10% purity) in EtOH (50 mL) was degassed and purged with H₂ for 3 times, and then the mixture was stirred at 15° C. for 2 hr under H₂ (15 Psi) atmosphere. The catalyst was removed by filtration through celite, which was then washed with MeOH (30 ml). The filtrate was concentrated to give compound tert-butyl 2-pyrrolidin-3-yloxyacetate (436 mg, 2.17 mmol, 72.66% yield) as yellow oil.

### Data:

LCMS (ESI⁺): m/z 202.1 (M+H)

¹H NMR (400 MHz, CHLOROFORM-d) δ=4.20-4.12 (m, 1H), 3.31 (br s, 2H), 3.23-3.11 (m, 2H), 3.00-2.88 (m, 2H), 1.93 (dt, J=4.0, 7.3 Hz, 2H), 1.52-1.46 (m, 9H)

### 3. General Procedure for Preparation of tert-butyl 2-[1-(2-prop-2-ynoxyacetyl) pyrrolidin-3-yl]oxy acetate

To a solution of tert-butyl 2-pyrrolidin-3-yloxyacetate (200 mg, 993.73 μmol) and 2-prop-2-ynoxyacetic acid (113.38 mg, 993.73 μmol) in DMF (4 mL) was added DIEA (256.87 mg, 1.99 mmol, 346.18 μL) and HATU (566.77 mg, 1.49 mmol) at 0° C. The mixture was stirred at 25° C. for 2 hr. The crude product was purified by reversed-phase HPLC (column: C18 20-35 um 100 A 80 g; mobile phase: [water-ACN]; B %: 0%-36% @ 60 mL/min) to give compound tert-butyl 2-[1-(2-prop-2-ynoxyacetyl)pyrrolidin-3-yl]oxy acetate (310 mg, 1.04 mmol, 52.46% yield) as a white solid.

### Data:

LCMS (ESI⁺): m/z 242.2 (M-56+H)

### 4. General Procedure for Preparation of 2-[1-(2-prop-2-ynoxyacetyl)pyrrolidin-3-yl]oxyacetic acid

To a solution of tert-butyl 2-[1-(2-prop-2-ynoxyacetyl)pyrrolidin-3-yl]oxyacetate (310 mg, 1.04 mmol) in DCM (3 mL) was added TFA (921.00 mg, 8.08 mmol, 0.6 mL). The mixture was stirred at 25° C. for 1 hr. The mixture concentrated under reduced pressure to give compound 2-[1-(2-prop-2-ynoxyacetyl)pyrrolidin-3-yl]oxyacetic acid (200 mg, 829.05 μmol, 79.52% yield, TFA) as yellow oil.

### Data:

LCMS (ESI⁺): m/z 242.0 (M+H)

### 5. General Procedure for Preparation of methyl (2S)-5,5-dimethyl-2-[[6-[3-[[1-[2-[1-(2-prop-2-ynoxyacetyl)pyrrolidin-3-yl]oxyacetyl]-4-piperidyl]oxy]phenoxy]pyridine-3-carbonyl]amino]hexanoate

To a solution of methyl (2S)-5,5-dimethyl-2-[[6-[3-(4-piperidyloxy)phenoxy]pyridine-3-carbonyl]amino]hexanoate (200 mg, 342.70 μmol, TFA) and 2-[1-(2-prop-2-ynoxyacetyl)pyrrolidin-3-yl]oxyacetic acid (82.67 mg, 342.70 μmol) in DMF (2 mL) was added DIEA (132.88 mg, 1.03 mmol, 179.08 μL) and HATU (260.61 mg, 685.41 μmol) at 0° C. The mixture was stirred at 15° C. for 1 hr. The crude product was purified by reversed-phase HPLC (column: C18 20-35 um 100 A 80 g; mobile phase: [water-ACN]; B %: 0%-65% @ 60 mL/min) to give the compound methyl (2S)-5,5-dimethyl-2-[[6-[3-[[1-[2-[1-(2-prop-2-ynoxyacetyl)pyrrolidin-3-yl]oxyacetyl]-4-piperidyl]oxy]phenoxy]pyridine-3-carbonyl]amino]hexanoate (60 mg, 86.61 μmol, 25.27% yield) as a white solid.

### Data:

LCMS (ESI⁺): m/z 693.4 (M+H)

### 6. General Procedure for Preparation of methyl (2S)-2-[[6-[3-[[1-[2-[1-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]acetyl]pyrrolidin-3-yl]oxyacetyl]-4-piperidyl]oxy]phenoxy]pyridine-3-carbonyl]amino]-5,5-dimethyl-hexanoate

To a solution of methyl (2S)-5,5-dimethyl-2-[[6-[3-[[1-[2-[1-(2-prop-2-ynoxyacetyl)pyrrolidin-3-yl]oxyacetyl]-4-piperidyl]oxy]phenoxy]pyridine-3-carbonyl]amino]hexanoate (55 mg, 79.39 μmol) and 3-[[6-[2-[(3R)-4-(2-azidoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (55.48 mg, 103.20 μmol) in H₂O (0.5 mL) and t-BuOH (0.5 mL) was added copper; sulfate (6.34 mg, 39.69 μmol, 6.09 μL) and sodium; (2R)-2-[(1S)-1,2-dihydroxyethyl]-4-hydroxy-5-oxo-2H-furan-3-olate (15.73 mg, 79.39 μmol). The mixture was stirred at 50° C. for 1 hr. The crude product was purified by reversed-phase HPLC (column: C18 20-35 um 100 A 80 g; mobile phase: [water-ACN]; B %: 0%-57% @ 60 mL/min) to give compound methyl (2S)-2-[[6-[3-[[1-[2-[1-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]acetyl]pyrrolidin-3-yl]oxyacetyl]-4-piperidyl]oxy]phenoxy]pyridine-3-carbonyl]amino]-5,5-dimethyl-hexanoate (90 mg, 73.15 μmol, 92.14% yield) as a white solid.

### Data:

LCMS (ESI⁺): m/z 616.4 (M/2+H)

### 7. General Procedure for Preparation of (2S)-2-[[6-[3-[[1-[2-[1-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]acetyl]pyrrolidin-3-yl]oxyacetyl]-4-piperidyl]oxy]phenoxy]pyridine-3-carbonyl]amino]-5,5-dimethyl-hexanoic acid

The deprotection of methyl (2S)-2-[[6-[3-[[1-[2-[1-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]acetyl]pyrrolidin-3-yl]oxyacetyl]-4-piperidyl]oxy]phenoxy]pyridine-3-carbonyl]amino]-5,5-dimethyl-hexanoate (45 mg, 36.57 μmol) was performed analogously to the description above of BF083 to give the compound of the title BF125 (6.5 mg, 5.24 μmol, 14.32% yield, 98% purity) as a yellow solid.

### Data:

LCMS (ESI⁺): m/z 608.8 (M/2+H)

¹H NMR (400 MHz, METHANOL-d4) δ=8.95 (d, J=1.3 Hz, 2H), 8.64-8.60 (m, 2H), 8.23 (td, J=2.3, 8.7 Hz, 1H), 8.05-7.99 (m, 2H), 7.95 (d, J=8.0 Hz, 1H), 7.61 (dd, J=4.8, 7.8 Hz, 1H), 7.36-7.28 (m, 2H), 6.99 (dd, J=3.5, 8.8 Hz, 1H), 6.87 (br d, J=8.3 Hz, 1H), 6.80 (d, J=2.0 Hz, 1H), 6.72 (br dd, J=2.1, 8.2 Hz, 1H), 5.70-5.53 (m, 1H), 5.50-5.38 (m, 1H), 5.12 (s, 2H), 4.85-4.68 (m, 5H), 4.64 (br s, 1H), 4.50 (br dd, J=4.8, 8.0 Hz, 1H), 4.40-4.30 (m, 1H), 4.27-4.15 (m, 5H), 3.91-3.64 (m, 4H), 3.62-3.49 (m, 4H), 3.47-3.36 (m, 3H), 3.18-3.04 (m, 1H), 2.26-2.08 (m, 1H), 2.07-1.89 (m, 4H), 1.84-1.69 (m, 3H), 1.39-1.28 (m, 10H), 1.15 (br d, J=5.6 Hz, 2H), 0.91 (s, 9H)

### BF126

### 1. General Procedure for Preparation of 2-prop-2-ynoxyethyl 4-methylbenzenesulfonate

To a solution of 2-prop-2-ynoxyethanol (2 g, 19.98 mmol, 1 eq) and TosCl (5.71 g, 29.97 mmol, 1.5 eq) in DCM (100 mL) was added TEA (4.04 g, 39.95 mmol, 5.56 mL, 2 eq) at 0° C. The mixture was stirred at 15° C. for 1 hr. The reaction mixture was quenched by addition H₂O 100 mL, and extracted with DCM 300 mL (100 mL*3). The combined organic layers were dried over Na₂SO₄, filtered and concentrated under reduced pressure. The residue was purified by flash silica gel chromatography (ISCO®; 220 g SepaFlash® Silica Flash Column, Eluent of 0˜25% Ethyl acetate/Petroleum ether gradient @ 120 mL/min) to give compound 2-prop-2-ynoxyethyl 4-methylbenzenesulfonate (21.5 g, 84.55 mmol, 84.64% yield) as a colorless oil.

### Data:

¹H NMR (400 MHz, CHLOROFORM-d) δ=7.81 (d, J=8.3 Hz, 2H), 7.35 (d, J=7.9 Hz, 2H), 4.22-4.17 (m, 2H), 4.14-4.11 (m, 2H), 3.76-3.70 (m, 2H), 2.45-2.45 (m, 1H), 2.45 (s, 2H), 2.43 (t, J=2.4 Hz, 1H)

### 2. General Procedure for Preparation of 3-(2-prop-2-ynoxyethoxy) propan-1-ol

To a solution of propane-1,3-diol (2 g, 26.28 mmol, 1.90 mL, 1 eq) in THF (20 mL) was added NaH (1.26 g, 31.54 mmol, 60% purity, 1.2 eq) at 0° C. The mixture was stirred at 0° C. for 0.5 hr. Then 2-prop-2-ynoxyethyl 4-methylbenzenesulfonate (5.35 g, 21.03 mmol, 0.8 eq) was added to the above mixture, the mixture was stirred at 60° C. for 12 hr. The reaction mixture was quenched by addition aq·NH₄Cl 20 mL, and extracted with Ethyl acetate 60 mL (20 mL*3). The combined organic layers were dried over Na₂SO₄, filtered and concentrated under reduced pressure. The residue was purified by flash silica gel chromatography (ISCO®; 80 g SepaFlash® Silica Flash Column, Eluent of 0˜50% Ethyl acetate/Petroleum ether gradient @ 80 mL/min) to give compound 3-(2-prop-2-ynoxyethoxy) propan-1-ol (540 mg, 3.41 mmol, 12.99% yield) as a yellow oil.

### Data:

¹H NMR (400 MHz, CHLOROFORM-d) δ=4.20 (d, J=2.4 Hz, 2H), 3.77 (t, J=5.6 Hz, 2H), 3.73-3.61 (m, 6H), 2.44 (t, J=2.4 Hz, 1H), 1.84 (quin, J=5.7 Hz, 2H)

### 3. General Procedure for Preparation of 1-iodo-3-(2-prop-2-ynoxyethoxy)propane

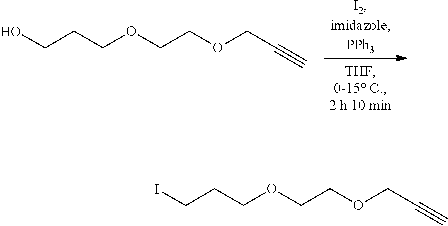

A mixture of 3-(2-prop-2-ynoxyethoxy) propan-1-ol (540 mg, 3.41 mmol, 1 eq), PPh₃ (1.07 g, 4.10 mmol, 1.2 eq) and IMIDAZOLE (278.86 mg, 4.10 mmol, 1.2 eq) in DCM (5 mL) was degassed and purged with N₂ for 3 times, and then the mixture was stirred at 0° C. for 10 min under N₂ atmosphere. Then 12 (1.04 g, 4.10 mmol, 825.12 UL, 1.2 eq) was added to the mixture at 0° C. The mixture was stirred at 15° C. for 2 h. The reaction mixture was quenched by addition H₂O 5 mL, and extracted with DCM 15 mL (5 mL*3). The combined organic layers were dried over Na₂SO₄, filtered and concentrated under reduced pressure. The residue was purified by flash silica gel chromatography (ISCO®; 40 g SepaFlash® Silica Flash Column, Eluent of 0˜10% Ethyl acetate/Petroleum ether gradient @ 70 mL/min) to give compound 1-iodo-3-(2-prop-2-ynoxyethoxy)propane (623 mg, 2.32 mmol, 68.08% yield) as a yellow oil.

### Data:

¹H NMR (400 MHz, CHLOROFORM-d) δ=4.22 (d, J=2.4 Hz, 2H), 3.74-3.61 (m, 4H), 3.55 (t, J=5.8 Hz, 2H), 3.29 (t, J=6.8 Hz, 2H), 2.45 (t, J=2.3 Hz, 1H), 2.12-2.04 (m, 2H)

### 4. General Procedure for Preparation of 3-(2-prop-2-ynoxyethoxy)propane-1-sulfonyl chloride

1-iodo-3-(2-prop-2-ynoxyethoxy)propane (300 mg, 1.12 mmol, 1 eq), thiourea (127.77 mg, 1.68 mmol, 1.5 eq) in EtOH (5 mL) was stirred for 1 hr at 80° C. TLC showed Reactant 1 was consumed and one new spot were detected, then the reaction was concentrated to give a residue, then the residue in ACN (1 mL) was added to a solution of NCS (747.13 mg, 5.60 mmol, 5 eq) in HCl (2 M, 391.66 μL, 0.7 eq) and ACN (5 mL) at 0° C., then the reaction was stirred for 1 hr at 15° C. The reaction mixture was diluted with H₂O 5 mL and extracted with DCM 15 mL (5 mL*3). The combined organic layers were dried over anhydrous Na₂SO₄, filtered and concentrated under reduced pressure to give compound 3-(2-prop-2-ynoxyethoxy)propane-1-sulfonyl chloride (270 mg, crude) as a black oil.

### 5. General Procedure for Preparation of methyl (2S)-5,5-dimethyl-2-[[6-[3-[3-[3-(2-prop-2-ynoxyethoxy) propylsulfonylamino]cyclobutoxy]phenoxy]pyridine-3-carbonyl]amino]hexanoate

To a solution of methyl (2S)-2-[[6-[3-(3-aminocyclobutoxy)phenoxy]pyridine-3-carbonyl]amino]-5,5-dimethyl-hexanoate (400.00 mg, 702.29 μmol, 1 eq, TFA) in DCM (5 mL) was added TEA (213.19 mg, 2.11 mmol, 293.25 μL, 3 eq), then 3-(2-prop-2-ynoxyethoxy)propane-1-sulfonyl chloride (253.56 mg, 1.05 mmol, 1.5 eq) in DCM (2 mL) was added at 0° C. and the reaction was stirred for 0.5 hr at 15° C. The reaction mixture was diluted with H₂O 3 mL and extracted with DCM 6 mL (2 mL*3). The combined organic layers were dried over anhydrous Na₂SO₄, filtered and concentrated under reduced pressure to give a residue. The residue was purified by prep-TLC (SiO₂, Petroleum ether: Ethyl acetate=1:2) to give compound methyl (2S)-5,5-dimethyl-2-[[6-[3-[3-[3-(2-prop-2-ynoxyethoxy) propylsulfonylamino]cyclobutoxy]phenoxy]pyridine-3-carbonyl]amino]hexanoate (140 mg, 212.19 μmol, 30.21% yield) as a yellow oil.

### Data:

LCMS (ESI+): m/z 660.4 (M+H)

### 6. General Procedure for Preparation of methyl (2S)-2-[[6-[3-[3-[3-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]propylsulfonylamino]cyclobutoxy]phenoxy]pyridine-3-carbonyl]amino]-5,5-dimethyl-hexanoate

To a solution of methyl (2S)-5,5-dimethyl-2-[[6-[3-[3-[3-(2-prop-2-ynoxyethoxy) propylsulfonylamino]cyclobutoxy]phenoxy]pyridine-3-carbonyl]amino]hexanoate (130 mg, 197.03 μmol, 1 eq) and 3-[[6-[2-[(3R)-4-(2-azidoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (127.10 mg, 236.44 μmol, 1.2 eq) in t-BuOH (1 mL) and H₂O (1 mL) was added copper; sulfate (31.45 mg, 197.03 μmol, 30.24 μL, 1 eq) and sodium; (2R)-2-[(1S)-1,2-dihydroxyethyl]-4-hydroxy-5-oxo-2H-furan-3-olate (39.03 mg, 197.03 μmol, 1 eq). The mixture was stirred at 50° C. for 0.5 hr. The mixture was concentrated under reduced pressure to give a residue. The mixture was diluted with water 5 mL, and then extracted with Ethyl acetate 15 mL (5 mL*3). The combined organic layers were washed with sat·NaCl 10 mL, dried over Na₂SO₄, filtered and concentrated under reduced pressure to give a residue. The residue was purified by prep-TLC (SiO₂, Ethyl acetate:Methanol=5:1) to give compound methyl (2S)-2-[[6-[3-[3-[3-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]propylsulfonylamino]cyclobutoxy]phenoxy]pyridine-3-carbonyl]amino]-5,5-dimethyl-hexanoate (114 mg, 95.21 μmol, 48.32% yield) as a yellow oil.

### Data:

LCMS (ESI+): m/z 1197.7 (M+H)

### 7. General Procedure for Preparation of (2S)-2-[[6-[3-[3-[3-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]propylsulfonylamino]cyclobutoxy]phenoxy]pyridine-3-carbonyl]amino]-5,5-dimethyl-hexanoic acid

The deprotection of methyl (2S)-2-[[6-[3-[3-[3-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]propylsulfonylamino]cyclobutoxy]phenoxy]pyridine-3-carbonyl]amino]-5,5-dimethyl-hexanoate (114 mg, 95.21 μmol, 1 eq) was performed analogously to the description above of BF083 to give the compound of the title BF126 (57.4 mg, 48.09 μmol, 50.51% yield, 99.134% purity) as a yellow solid.

### Data:

LCMS (ESI⁺): m/z 1183.3 (M+H)

¹H NMR (400 MHz, METHANOL-d4) δ ppm 8.95 (s, 2H) 8.57-8.65 (m, 2H) 8.22 (dd, J=8.70, 2.50 Hz, 1H) 8.00-8.06 (m, 1H) 7.90-7.99 (m, 2H) 7.61 (dd, J=8.11, 4.77 Hz, 1H) 7.25-7.35 (m, 2H) 6.97 (d, J=8.58 Hz, 1H) 6.72 (ddd, J=13.71, 8.17, 1.73 Hz, 2H) 6.66 (t, J=2.21 Hz, 1H) 5.49-5.67 (m, 1H) 5.35-5.47 (m, 1H) 5.12 (s, 2H) 4.59-4.83 (m, 6H) 4.50 (dd, J=8.82, 5.13 Hz, 1H) 4.28-4.45 (m, 2H) 3.81-3.90 (m, 0.5H) 3.64-3.69 (m, 2H) 3.50-3.63 (m, 6H) 3.36-3.40 (m, 0.5H) 3.06-3.11 (m, 2H) 2.85-2.97 (m, 2H) 2.04-2.14 (m, 2H) 1.89-2.02 (m, 3H) 1.75-1.87 (m, 1H) 1.32-1.42 (m, 8H) 1.28-1.32 (m, 2H) 1.15 (br d, J=6.20 Hz, 2H) 0.91 (s, 9H).

### BF131

### 1. General Procedure for Preparation of tert-butyl 2-(3-hydroxyphenoxy)acetate

To a solution of tert-butyl 2-bromoacetate (17.71 g, 90.82 mmol, 13.41 mL, 1 eq) and benzene-1,3-diol (10 g, 90.82 mmol, 15.15 mL, 1 eq) in ACN (200 mL) was added K₂CO₃ (12.55 g, 90.82 mmol, 1 eq). The mixture was stirred at 80° C. for 12 hr. The reaction mixture was quenched by addition H₂O 100 mL, and extracted with Ethyl acetate 300 mL (100 mL*3). The combined organic layers were washed with brine 200 mL (100 mL*2) dried over Na₂SO₄, filtered and concentrated under reduced pressure. The residue was purified by column chromatography (SiO₂, Petroleum ether/Ethyl acetate=50/1 to 3/1) to give compound tert-butyl 2-(3-hydroxyphenoxy)acetate (29.54 g, 131.73 mmol, 48.35% yield) as a yellow oil.

### Data:

LCMS (ESI⁺): m/z 225.0 (M+H)

### 2. General Procedure for Preparation of tert-butyl 2-[3-[(5-bromo-2-pyridyl)oxy]phenoxy]acetate

To a solution of tert-butyl 2-(3-hydroxyphenoxy)acetate (15 g, 66.89 mmol, 1 eq) in ACN (150 mL) was added 5-bromo-2-fluoro-pyridine (14.13 g, 80.27 mmol, 8.26 mL, 1.2 eq) and Cs₂CO₃ (43.59 g, 133.78 mmol, 2 eq). The mixture was stirred at 80° C. for 2 hr. The mixture was diluted with water 100 mL, and then extracted with Ethyl acetate 300 mL (100 mL*3), dried over Na₂SO₄, filtered and concentrated under reduced pressure to give a residue. The residue was purified by flash silica gel chromatography (ISCO®; 80 g SepaFlash® Silica Flash Column, Eluent of 0˜10% Ethyl acetate/Petroleum ether gradient @ 60 mL/min) to give compound tert-butyl 2-[3-[(5-bromo-2-pyridyl)oxy]phenoxy]acetate (18.52 g, 48.71 mmol, 72.82% yield) as a colorless liquid.

### Data:

LCMS (ESI⁺): m/z 380.1 (M+H)

### 3. General Procedure for Preparation of tert-butyl 2-[3-[[5-(4,4,5,5-tetramethyl-1,3,2-dioxaborolan-2-yl)-2-pyridyl]oxy]phenoxy]acetate

To a solution of tert-butyl 2-[3-[(5-bromo-2-pyridyl)oxy]phenoxy]acetate (1 g, 2.63 mmol, 1 eq) in dioxane (10 mL) was added 4,4,5,5-tetramethyl-2-(4,4,5,5-tetramethyl-1,3,2-dioxaborolan-2-yl)-1,3,2-dioxaborolane (1.00 g, 3.94 mmol, 1.5 eq) and KOAc (774.33 mg, 7.89 mmol, 3 eq) and Pd(dppf)Cl₂ (192.44 mg, 263.00 μmol, 0.1 eq). The mixture was stirred at 95° C. for 2 hr. The mixture was diluted with water 100 mL, and then extracted with Ethyl acetate 300 mL (100 mL*3), dried over Na₂SO₄, filtered and concentrated under reduced pressure to give compound tert-butyl 2-[3-[5-(4,4,5,5-tetramethyl-1,3,2-dioxaborolan-2-yl)-2-pyridyl]oxy]phenoxylacetate (1.5 g, crude) as a colorless liquid.

### Data:

LCMS (ESI⁺): m/z 428.2 (M+H)

### 4. General Procedure for Preparation of tert-butyl 2-[3-[(5-hydroxy-2-pyridyl)oxy]phenoxy]acetate

To a solution of tert-butyl 2-[3-[5-(4,4,5,5-tetramethyl-1,3,2-dioxaborolan-2-yl)-2-pyridyl]oxy]phenoxylacetate (1.5 g, 3.51 mmol, 1 eq) in THF (1 mL) added sodium; 3-oxidodioxaborirane; tetrahydrate (1.62 g, 10.53 mmol, 2.03 mL, 3 eq) in H₂O (0.5 mL). The mixture was stirred at 20° C. for 2 hr. The mixture was diluted with water 100 mL, and then extracted with Ethyl acetate 300 mL (100 mL*3), dried over Na₂SO₄, filtered and concentrated under reduced pressure to give a residue. The residue was purified by flash silica gel chromatography (ISCO®; 80 g SepaFlash® Silica Flash Column, Eluent of 0˜10% Ethyl acetate/Petroleum ether gradient @ 60 mL/min) to give compound tert-butyl 2-[3-[(5-hydroxy-2-pyridyl)oxy]phenoxy]acetate (0.69 g, 2.17 mmol, 61.94% yield) as a colorless liquid.

### Data:

LCMS (ESI⁺): m/z 318.1 (M+H)

### 5. General Procedure for Preparation of benzyl 4-[[6-[3-(2-tert-butoxy-2-oxo-ethoxy)phenoxy]-3-pyridyl]oxy]piperidine-1-carboxylate

To a mixture of tert-butyl 2-[3-[(5-hydroxy-2-pyridyl)oxy]phenoxy]acetate (600 mg, 1.89 mmol, 1 eq) and benzyl 4-(p-tolylsulfonyloxy) piperidine-1-carboxylate (883.65 mg, 2.27 mmol, 1.2 eq) in DMF (15 mL) was add Cs₂CO₃ (1.23 g, 3.78 mmol, 2 eq), and then the mixture was stirred at 80° C. for 12 hr. The mixture was diluted with water 15 mL, and then extracted with Ethyl acetate 45 mL (15 mL*3). The combined organic layers were washed with sat·NaCl 20 mL*2, dried over Na₂SO₄, filtered and concentrated under reduced pressure to give a residue. The residue was purified by column chromatography (SiO2, Petroleum ether/Ethyl acetate=1/to 3/1) to give compound benzyl 4-[[6-[3-(2-tert-butoxy-2-oxo-ethoxy)phenoxy]-3-pyridyl]oxy]piperidine-1-carboxylate (660 mg, 1.23 mmol, 65.30% yield) as a yellow oil.

### Data:

LCMS (ESI⁺): m/z 535.4 (M+H)

### 6. General Procedure for Preparation of 2-[3-[[5-[(1-benzyloxycarbonyl-4-piperidyl)oxy]-2-pyridyl]oxy]phenoxy]acetic acid

A solution of benzyl 4-[[6-[3-(2-tert-butoxy-2-oxo-ethoxy)phenoxy]-3-pyridyl]oxy]piperidine-1-carboxylate (660 mg, 1.23 mmol, 1 eq) in DCM (6 mL) and TFA (1.2 mL) was stirred at 15° C. for 8 hr. The DCM and TFA was removed under reduced pressure to give compound 2-[3-[[5-[(1-benzyloxycarbonyl-4-piperidyl)oxy]-2-pyridyl]oxy]phenoxy]acetic acid (730 mg, crude, TFA) as a yellow oil

### Data:

LCMS (ESI⁺): m/z 479.3 (M+H)

### 7. General Procedure for Preparation of benzyl 4-[[6-[3-[2-[[(1S)-1-methoxycarbonyl-4,4-dimethyl-pentyl]amino]-2-oxo-ethoxy]phenoxy]-3-pyridyl]oxy]piperidine-1-carboxylate

To a solution of 2-[3-[[5-[(1-benzyloxycarbonyl-4-piperidyl)oxy]-2-pyridyl]oxy]phenoxy]acetic acid (710 mg, 1.20 mmol, 1 eq, TFA) and methyl (2S)-2-amino-5,5-dimethyl-hexanoate (207.60 mg, 1.20 mmol, 1 eq) in DMF (10 mL) was added DIEA (309.74 mg, 2.40 mmol, 417.44 UL, 2 eq) and HATU (683.43 mg, 1.80 mmol, 921.07 μL, 1.5 eq) at 0° C. The mixture was stirred at 15° C. for 1 hr. The mixture was diluted with water 10 mL, and then extracted with Ethyl acetate 30 mL (10 mL*3). The combined organic layers were washed with sat. NaCl 10 mL*2, dried over Na₂SO₄, filtered and concentrated under reduced pressure to give a residue. The residue was purified by prep-TLC (SiO₂, Petroleum ether: Ethyl acetate=1:1) to give compound methyl benzyl 4-[[6-[3-[2-[[(1S)-1-methoxycarbonyl-4,4-dimethyl-pentyl]amino]-2-oxo-ethoxy]phenoxy]-3-pyridyl]oxy]piperidine-1-carboxylate (640 mg, 1.01 mmol, 84.28% yield) as a yellow oil.

### Data:

LCMS (ESI⁺): m/z 634.5 (M+H)

### 8. General Procedure for Preparation of methyl (2S)-5,5-dimethyl-2-[[2-[3-[[5-(4-piperidyloxy)-2-pyridyl]oxy]phenoxy]acetyl]amino]hexanoate

To a solution of benzyl 4-[[6-[3-[2-[[(1S)-1-methoxycarbonyl-4,4-dimethyl-pentyl]amino]-2-oxo-ethoxy]phenoxy]-3-pyridyl]oxy]piperidine-1-carboxylate (640 mg, 1.01 mmol, 1 eq) in THF (10 mL) was added Pd/C (10% purity, 1.00 eq) under N₂ atmosphere. The suspension was degassed and purged with H₂ for 3 times. The mixture was stirred under H₂ (15 Psi) at 15° C. for 1 hr. The catalyst was removed by filtration through celite, which was then washed with Ethyl acetate (150 ml). The filtrate was concentrated to give compound methyl (2S)-5,5-dimethyl-2-[[2-[3-[[5-(4-piperidyloxy)-2-pyridyl]oxy]phenoxy]acetyl]amino]hexanoate (500 mg, crude) as a yellow oil.

### Data:

LCMS (ESI⁺): m/z 500.3 (M+H)

¹H NMR (400 MHz, CHLOROFORM-d) 0 ppm 7.90 (d, J=2.88 Hz, 1H) 7.28-7.34 (m, 2H) 7.00 (br d, J=8.13 Hz, 1H) 6.89 (d, J=8.88 Hz, 1H) 6.70-6.80 (m, 3H) 4.67 (td, J=7.69, 5.25 Hz, 1H) 4.51 (d, J=1.63 Hz, 2H) 4.31 (td, J=7.75, 3.88 Hz, 1H) 3.76 (s, 3H) 3.10-3.22 (m, 2H) 2.71-2.80 (m, 2H) 2.04 (br s, 2H) 1.84-1.93 (m, 1H) 1.63-1.75 (m, 3H) 1.09-1.21 (m, 2H) 0.86 (s, 9H)

### 9. General Procedure for Preparation of methyl (2S)-5,5-dimethyl-2-[[2-[3-[[5-[[1-[2-[2-(2-prop-2-ynoxyethoxy)ethoxy]acetyl]-4-piperidyl]oxy]-2-pyridyl]oxy]phenoxy]acetyl]amino]hexanoate

To a solution of methyl (2S)-5,5-dimethyl-2-[[2-[3-[[5-(4-piperidyloxy)-2-pyridyl]oxy]phenoxy]acetyl]amino]hexanoate (250 mg, 500.40 μmol, 1 eq) and 2-[2-(2-prop-2-ynoxyethoxy)ethoxy]acetic acid (101.18 mg, 500.40 μmol, 1 eq) in DMF (4 mL) was added DIEA (129.35 mg, 1.00 mmol, 174.32 μL, 2 eq) and HATU (285.40 mg, 750.60 μmol, 185.93 μL, 1.5 eq) at 0° C. The mixture was stirred at 15° C. for 0.5 hr. The mixture was diluted with water 5 mL, and then extracted with Ethyl acetate 15 mL (5 mL*3). The combined organic layers were washed with sat. NaCl 10 mL, dried over Na₂SO₄, filtered and concentrated under reduced pressure to give a residue. The residue was purified by prep-TLC (SiO₂, Petroleum ether: Ethyl acetate=1:1) to give (2S)-5,5-dimethyl-2-[[2-[3-[[5-[[1-[2-[2-(2-prop-2-compound methyl ynoxyethoxy)ethoxy]acetyl]-4-piperidyl]oxy]-2-pyridyl]oxy]phenoxy]acetyl]amino]hexanoate (270 mg, 394.86 μmol, 78.91% yield) as a yellow oil.

### Data:

LCMS (ESI⁺): m/z 684.5 (M+H)

### 10. General Procedure for Preparation of methyl (2S)-2-[[2-[3-[[5-[[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]-4-piperidyl]oxy]-2-pyridyl]oxy]phenoxy]acetyl]amino]-5,5-dimethyl-hexanoate

To a solution of methyl (2S)-5,5-dimethyl-2-[[2-[3-[[5-[[1-[2-[2-(2-prop-2-ynoxyethoxy)ethoxy]acetyl]-4-piperidyl]oxy]-2-pyridyl]oxy]phenoxy]acetyl]amino]hexanoate (120 mg, 175.49 μmol, 1 eq) and 3-[[6-[2-[(3R)-4-(2-azidoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (148.25 mg, 193.04 μmol, 1.1 eq) in t-BuOH (1 mL) and H₂O (1 mL) was added copper; sulfate (14.01 mg, 87.75 μmol, 13.47 μL, 0.5 eq) and sodium; (2R)-2-[(1S)-1,2-dihydroxyethyl]-4-hydroxy-5-oxo-2H-furan-3-olate (34.77 mg, 175.49 μmol, 1 eq). The mixture was stirred at 50° C. for 0.5 hr. The mixture was filtered and the filtrate was purified by reversed-phase HPLC (column: C18 20-35 um 100 A 80 g; mobile phase: [water-ACN]; B %: 0%-60% @ 80 mL/min) to give compound methyl (2S)-2-[[2-[3-[[5-[[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]-4-piperidyl]oxy]-2-pyridyl]oxy]phenoxy]acetyl]amino]-5,5-dimethyl-hexanoate (140 mg, 114.63 μmol, 65.32% yield) as a yellow solid.

### Data:

LCMS (ESI⁺): m/z 1221.7 (M+H)

### 11. General Procedure for Preparation of (2S)-2-[[2-[3-[[5-[[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]-4-piperidyl]oxy]-2-pyridyl]oxy]phenoxy]acetyl]amino]-5,5-dimethyl-hexanoic acid

The deprotection of methyl (2S)-2-[[2-[3-[[5-[[1-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]acetyl]-4-piperidyl]oxy]-2-pyridyl]oxy]phenoxy]acetyl]amino]-5,5-dimethyl-hexanoate (140 mg, 114.63 μmol, 1 eq) was performed analogously to the description above of BF083 to give the compound of the title BF131 (81.4 mg, 67.11 μmol, 58.55% yield, 99.54% purity) as a yellow solid.

### Data:

LCMS (ESI⁺): m/z 1207.3 (M+H)

¹H NMR (400 MHz, METHANOL-d4) δ ppm 8.96 (s, 2H) 8.60-8.65 (m, 1H) 8.00-8.05 (m, 1H) 7.91-8.00 (m, 2H) 7.87 (d, J=2.93 Hz, 1H) 7.61 (dd, J=8.13, 4.71 Hz, 1H) 7.51 (dd, J=8.86, 3.00 Hz, 1H) 7.24-7.33 (m, 2H) 6.91 (d, J=8.93 Hz, 1H) 6.79 (dd, J=8.31, 1.96 Hz, 1H) 6.72 (t, J=2.20 Hz, 1H) 6.67 (dd, J=8.07, 1.59 Hz, 1H) 5.51-5.71 (m, 1H) 5.36-5.50 (m, 1H) 5.12 (s, 2H) 4.68-4.83 (m, 2H) 4.66 (s, 2H) 4.57-4.63 (m, 1H) 4.56 (s, 2H) 4.38-4.45 (m, 1H) 4.31-4.38 (m, 1H) 4.21-4.31 (m, 2H) 3.63-3.90 (m, 11H) 3.33-3.60 (m, 4H) 2.99-3.19 (m, 1H) 1.82-2.06 (m, 3H) 1.63-1.81 (m, 3H) 1.36 (s, 6H) 1.30 (br d, J=5.01 Hz, 1H) 1.09-1.24 (m, 4H) 0.85 (s, 9H)

### BF129

### 1. General Procedure for Preparation of tert-butyl 3-prop-2-ynoxypyrrolidine-1-carboxylate

To a solution of tert-butyl 3-hydroxypyrrolidine-1-carboxylate (3 g, 16.02 mmol, 1 eq) in THF (30 mL) was added NaH (961.26 mg, 24.03 mmol, 60% purity, 1.5 eq) at 0° C. Then 3-bromoprop-1-yne (2.86 g, 19.23 mmol, 2.07 mL, 1.2 eq) was added to the mixture after stirred for 30 min. The mixture was stirred at 20° C. for 0.5 h. The reaction mixture was quenched by addition NH₄Cl 30 mL, and extracted with EtOAc 90 mL (30 mL*3). The combined organic layers were dried over Na₂SO₄, filtered and concentrated under reduced pressure. The residue was purified by column chromatography (SiO₂, Petroleum ether/Ethyl acetate=50/1 to 10/1) to give compound tert-butyl 3-prop-2-ynoxypyrrolidine-1-carboxylate (3.6 g, 15.98 mmol, 99.73% yield) as yellow oil.

### Data:

LCMS (ESI⁺): m/z 170.1 (M-56+H)

¹H NMR (400 MHz, CHLOROFORM-d) δ=4.27 (br s, 1H), 4.16 (br d, J=2.2 Hz, 2H), 3.53-3.31 (m, 4H), 2.47-2.39 (m, 1H), 2.07-1.89 (m, 2H), 1.45 (s, 9H)

### 2. General Procedure for Preparation of 3-prop-2-ynoxypyrrolidine

To a solution of tert-butyl 3-prop-2-ynoxypyrrolidine-1-carboxylate (0.7 g, 3.11 mmol, 1 eq) in DCM (10 mL) was added TFA (2 mL). The mixture was stirred at 20° C. for 1 h. 5 reactions were combined for monitor, work-up and purification. The reaction mixture was concentrated under reduced pressure to give compound 3-prop-2-ynoxypyrrolidine (6.17 g, crude, TFA) as yellow oil.

### Data:

¹H NMR (400 MHz, CHLOROFORM-d) δ=4.53 (t, J=4.2 Hz, 1H), 4.19 (d, J=2.4 Hz, 2H), 3.61-3.45 (m, 3H), 3.44-3.32 (m, 1H), 2.49 (t, J=2.3 Hz, 1H), 2.36-2.25 (m, 1H), 2.19-2.07 (m, 1H)

### 3. General Procedure for Preparation of 2-[2-oxo-2-(3-prop-2-ynoxypyrrolidin-1-yl)ethoxy]acetic acid

To a solution of 3-prop-2-ynoxypyrrolidine (1 g, 4.18 mmol, 1 eq, TFA) in THF (10 mL) was added TEA (846.09 mg, 8.36 mmol, 1.16 mL, 2 eq) and 1,4-dioxane-2,6-dione (485.27 mg, 4.18 mmol, 1 eq). The mixture was stirred at 20° C. for 1 h. The mixture was acidified by HCl (1 M) till pH=3-4, filtered and the filter cake was dried in vacuum to give the crude product. The residue was purified by prep-HPLC (HCl Condition; column: Phenomenex luna C18 250*50 mm*10 um; mobile phase: [H₂O (0.05% HCl)-ACN]; gradient: 1%-40% B over 10.0 min) to give compound 2-[2-oxo-2-(3-prop-2-ynoxypyrrolidin-1-yl)ethoxy]acetic acid (1 g, 4.15 mmol, 99.15% yield) was obtained as yellow oil.

### Data:

LCMS (ESI⁺): m/z 242.1 (M+H)

¹H NMR (400 MHz, CHLOROFORM-d) δ=4.39-4.29 (m, 3H), 4.25-4.16 (m, 4H), 3.74 (ddd, J=2.9, 9.0, 11.9 Hz, 1H), 3.66-3.56 (m, 1H), 3.55-3.40 (m, 3H), 2.48 (q, J=2.5 Hz, 1H), 2.35-2.23 (m, 1H), 2.20-2.03 (m, 1H), 1.96 (dtd, J=4.6, 9.2, 13.8 Hz, 1H)

### 4. General Procedure for Preparation of methyl (2S)-5,5-dimethyl-2-[[6-[3-[[1-[2-[2-oxo-2-(3-prop-2-ynoxypyrrolidin-1-yl)ethoxy]acetyl]-4-piperidyl]oxy]phenoxy]pyridine-3-carbonyl]amino]hexanoate

To a solution of methyl (2S)-5,5-dimethyl-2-[[6-[3-(4-piperidyloxy)phenoxy]pyridine-3-carbonyl]amino]hexanoate (150 mg, 319.44 μmol, 1 eq) in DMF (2 mL) was added 2-[2-oxo-2-(3-prop-2-ynoxypyrrolidin-1-yl)ethoxy]acetic acid (115.59 mg, 479.16 μmol, 1.5 eq) and DIEA (123.86 mg, 958.32 μmol, 166.92 μL, 3 eq). The mixture was cooled to 0° C. To the mixture was added HATU (182.19 mg, 479.16 μmol, 1.5 eq) and stirred at 20° C. for 1 h. The mixture was filtered and the filtrate was purified by prep-HPLC. The crude product was purified by reversed-phase HPLC flash C18 gel chromatography (ISCO; 40 g SepaFlash C18 Flash Column, eluent of 0-100% MeCN/H₂O 60 ml/min) to give compound methyl (2S)-5,5-dimethyl-2-[[6-[3-[[1-[2-[2-oxo-2-(3-prop-2-ynoxypyrrolidin-1-yl)ethoxy]acetyl]-4-piperidyl]oxy]phenoxy]pyridine-3-carbonyl]amino]hexanoate (51.9 mg, 74.91 μmol, 23.45% yield) as a white solid.

### Data:

LCMS (ESI⁺): m/z 693.4 (M+H)

### 5. General Procedure for Preparation of methyl (2S)-2-[[6-[3-[[1-[2-[2-[3-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]pyrrolidin-1-yl]-2-oxo-ethoxy]acetyl]-4-piperidyl]oxy]phenoxy]pyridine-3-carbonyl]amino]-5,5-dimethyl-hexanoate

To a solution of methyl (2S)-5,5-dimethyl-2-[[6-[3-[[1-[2-[2-oxo-2-(3-prop-2-ynoxypyrrolidin-1-yl)ethoxy]acetyl]-4-piperidyl]oxy]phenoxy]pyridine-3-carbonyl]amino]hexanoate (35 mg, 50.52 μmol, 1 eq) and 3-[[6-[2-[(3R)-4-(2-azidoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (27.16 mg, 50.52 μmol, 1 eq) in t-BuOH (1 mL) and H₂O (1 mL) was added copper; sulfate (4.03 mg, 25.26 μmol, 3.88 μL, 0.5 eq) and sodium; (2R)-2-[(1S)-1,2-dihydroxyethyl]-4-hydroxy-5-oxo-2H-furan-3-olate (10.01 mg, 50.52 μmol, 1 eq). The mixture was stirred at 50° C. for 1 h. The mixture was filtered and the filtrate was purified by prep-HPLC. The crude product was purified by reversed-phase HPLC flash C18 gel chromatography (ISCO; 40 g SepaFlash C18 Flash Column, eluent of 0-100% ACN/H₂O 60 ml/min) to give compound methyl (2S)-2-[[6-[3-[[1-[2-[2-[3-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]pyrrolidin-1-yl]-2-oxo-ethoxy]acetyl]-4-piperidyl]oxy]phenoxy]pyridine-3-carbonyl]amino]-5,5-dimethyl-hexanoate (40.9 mg, 33.24 μmol, 65.80% yield) as yellow oil.

### Data:

LCMS (ESI⁺): m/z 616.1 (M/2+H)

### 6. General Procedure for Preparation of (2S)-2-[[6-[3-[[1-[2-[2-[3-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]pyrrolidin-1-yl]-2-oxo-ethoxy]acetyl]-4-piperidyl]oxy]phenoxy]pyridine-3-carbonyl]amino]-5,5-dimethyl-hexanoic acid

The deprotection of methyl (2S)-2-[[6-[3-[[1-[2-[2-[3-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]pyrrolidin-1-yl]-2-oxo-ethoxy]acetyl]-4-piperidyl]oxy]phenoxy]pyridine-3-carbonyl]amino]-5,5-dimethyl-hexanoate (35 mg, 28.45 μmol, 1 eq) was performed analogously to the description above of BF083 to give the compound of the title BF129 (16.1 mg, 12.81 μmol, 45.04% yield, 96.801% purity) as a yellow solid.

### Data:

LCMS (ESI⁺): m/z 1216.3 (M+H)

¹H NMR (400 MHz, METHANOL-d4) δ=8.96 (s, 2H), 8.62 (d, J=2.7 Hz, 2H), 8.27-8.20 (m, 1H), 8.03 (d, J=8.2 Hz, 1H), 7.95 (d, J=7.9 Hz, 2H), 7.61 (dd, J=4.7, 8.0 Hz, 1H), 7.36-7.28 (m, 2H), 6.99 (br d, J=8.7 Hz, 1H), 6.88 (dd, J=2.1, 8.5 Hz, 1H), 6.79 (s, 1H), 6.72 (br d, J=8.3 Hz, 1H), 5.71-5.51 (m, 1H), 5.48-5.37 (m, 1H), 5.12 (s, 2H), 4.83-4.59 (m, 6H), 4.50 (dd, J=5.2, 8.7 Hz, 1H), 4.40-4.31 (m, 3H), 4.29-4.20 (m, 2H), 3.91-3.36 (m, 11H), 3.19-3.01 (m, 1H), 2.27-2.07 (m, 1H), 2.06-1.90 (m, 4H), 1.86-1.71 (m, 3H), 1.42-1.27 (m, 10H), 1.16 (br d, J=5.5 Hz, 2H), 0.91 (s, 9H)

### BF133

### 1. General Procedure for Preparation of methyl (2S)-5,5-dimethyl-2-[[2-[3-[[5-[[1-[3-(2-prop-2-ynoxyethoxy) propanoyl]-4-piperidyl]oxy]-2-pyridyl]oxy]phenoxy]acetyl]amino]hexanoate

To a solution of methyl (2S)-5,5-dimethyl-2-[[2-[3-[[5-(4-piperidyloxy)-2-pyridyl]oxy]phenoxy]acetyl]amino]hexanoate (80 mg, 160.13 μmol, 1 eq), 3-(2-prop-2-ynoxyethoxy) propanoic acid (41.36 mg, 240.19 μmol, 1.5 eq) in DMF (2 mL) was added HATU (91.33 mg, 240.19 μmol, 1.5 eq) and DIEA (62.09 mg, 480.39 μmol, 83.67 μL, 3 eq) at 0° C., then the reaction was stirred for 0.5 hr at 20° C. The reaction mixture was partitioned between water 2 mL and Ethyl acetate 6 mL (2 mL*3). The organic phase was separated, washed with brine 9 mL (3 mL*3), dried over [Na₂SO₄], filtered and concentrated under reduced pressure to give a residue. The residue was purified by flash silica gel chromatography (ISCO®; 12 g SepaFlash® Silica Flash Column, Eluent of 0˜35% Ethyl acetate/Petroleum ether gradient @ 50 mL/min) to give compound methyl (2S)-5, 5-dimethyl-2-[[2-[3-[[5-[[1-[3-(2-prop-2-ynoxyethoxy) propanoyl]-4-piperidyl]oxy]-2-pyridyl]oxy]phenoxy]acetyl]amino]hexanoate (0.1 g, 152.96 μmol, 95.52% yield) as a yellow oil.

### Data:

LCMS (ESI+): m/z 654.5 (M+H)

### 2. General Procedure for Preparation of methyl (2S)-2-[[2-[3-[[5-[[1-[3-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]propanoyl]-4-piperidyl]oxy]-2-pyridyl]oxy]phenoxy]acetyl]amino]-5,5-dimethyl-hexanoate

To a solution of themethyl (2S)-5,5-dimethyl-2-[[2-[3-[[5-[[1-[3-(2-prop-2-ynoxyethoxy) propanoyl]-4-piperidyl]oxy]-2-pyridyl]oxy]phenoxy]acetyl]amino]hexanoate (90 mg, 137.67 μmol, 1 eq) and 3-[[6-[2-[(3R)-4-(2-azidoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (96.21 mg, 178.96 μmol, 1.3 eq) in t-BuOH (0.5 mL) and H₂O (0.5 mL) was added copper; sulfate (21.97 mg, 137.67 μmol, 21.13 μL, 1 eq) and sodium; (2R)-2-[(1S)-1,2-dihydroxyethyl]-4-hydroxy-5-oxo-2H-furan-3-olate (27.27 mg, 137.67 μmol, 1 eq). The reaction was stirred for 1 hr at 50° C. The reaction mixture was partitioned between water 2 mL and Ethyl acetate 2 mL the organic phase was separated, washed with brine 30 mL (10 mL*3), dried over [Na₂SO₄], filtered and concentrated under reduced pressure to give a residue. The crude product was purified by reversed-phase HPLC (ISCO; 80 g SepaFlash C18 Flash Column, eluent of 0-100% MeCN/H₂O 80 ml/min) to give compound methyl (2S)-2-[[2-[3-[[5-[[1-[3-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]propanoyl]-4-piperidyl]oxy]-2-pyridyl]oxy]phenoxy]acetyl]amino]-5,5-dimethyl-hexanoate (0.1 g, 83.94 μmol, 60.97% yield) as a yellow oil.

### Data:

LCMS (ESI⁺): m/z 1191.7 (M+H)

### 3. General Procedure for Preparation of (2S)-2-[[2-[3-[[5-[[1-[3-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]propanoyl]-4-piperidyl]oxy]-2-pyridyl]oxy]phenoxy]acetyl]amino]-5,5-dimethyl-hexanoic acid

The deprotection of methyl (2S)-2-[[2-[3-[[5-[[1-[3-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]propanoyl]-4-piperidyl]oxy]-2-pyridyl]oxy]phenoxy]acetyl]amino]-5,5-dimethyl-hexanoate (90 mg, 75.55 μmol, 1 eq) was performed analogously to the description above of BF083 to give the compound of the title BF133 (85.3 mg, 72.45 μmol, 95.91% yield, 100% purity) as a yellow solid.

### Data:

LCMS (ESI⁺): m/z 1177.7 (M+H)

¹H NMR (400 MHz, METHANOL-d4) δ=8.96 (s, 2H), 8.63 (dd, J=1.2, 4.6 Hz, 1H), 8.06-8.01 (m, 1H), 7.95 (d, J=7.9 Hz, 2H), 7.88 (d, J=2.8 Hz, 1H), 7.62 (dd, J=4.8, 8.1 Hz, 1H), 7.51 (dd, J=3.1, 8.9 Hz, 1H), 7.33-7.24 (m, 2H), 6.92 (d, J=8.9 Hz, 1H), 6.81-6.75 (m, 1H), 6.72 (t, J=2.1 Hz, 1H), 6.67 (dd, J=1.4, 8.1 Hz, 1H), 5.71-5.53 (m, 1H), 5.51-5.36 (m, 1H), 5.12 (s, 2H), 4.84-4.69 (m, 2H), 4.69-4.51 (m, 6H), 4.42 (dd, J=4.8, 8.2 Hz, 1H), 4.38-4.28 (m, 1H), 3.87-3.79 (m, 2H), 3.76 (t, J=6.1 Hz, 2H), 3.71-3.66 (m, 2H), 3.65-3.60 (m, 2H), 3.60-3.41 (m, 3H), 3.38 (br d, J=5.5 Hz, 1H), 3.23-3.00 (m, 1H), 2.81-2.69 (m, 1H), 2.67-2.56 (m, 1H), 2.04-1.98 (m, 1H), 1.96-1.83 (m, 2H), 1.80-1.64 (m, 3H), 1.36 (s, 6H), 1.30 (br d, J=5.5 Hz, 1H), 1.23-1.08 (m, 4H), 0.85 (s, 9H)

### BF137

### 1. General Procedure for Preparation of [2-[2-[2-[2-[2-(tert-butoxycarbonylamino)ethoxy]ethoxy]ethoxy]ethylamino]pyrimidin-5-yl]boronic acid

To a solution of tert-butyl N-[2-[2-[2-(2-aminoethoxy)ethoxy]ethoxy]ethyl]carbamate (1 g, 3.42 mmol, 1 eq) and 2-chloro-5-(4,4,5,5-tetramethyl-1,3,2-dioxaborolan-2-yl)pyrimidine (904.82 mg, 3.76 mmol, 1.1 eq) in EtOH (10 mL) was added TEA (1.04 g, 10.26 mmol, 1.43 mL, 3 eq). The mixture was stirred at 80° C. for 1 h. The mixture was diluted with water 5 mL, and then extracted with Ethyl acetate 15 mL (5 mL*3). The combined organic layers were washed with sat·NaCl 10 mL, dried over Na₂SO₄, filtered and concentrated under reduced pressure to give compound [2-[2-[2-[2-[2-(tert-butoxycarbonylamino)ethoxy]ethoxy]ethoxy]ethylamino]pyrimidin-5-yl]boronic acid (1 g, crude) as yellow oil.

### Data:

LCMS (ESI⁺): m/z 414.9 (M+H)

### 2. General Procedure for Preparation of tert-butyl N-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethoxy]ethyl]carbamate

A mixture of [2-[2-[2-[2-[2-(tert-butoxycarbonylamino)ethoxy]ethoxy]ethoxy]ethylamino]pyrimidin-5-yl]boronic acid (200 mg, 482.79 μmol, 1 eq), 3-[(6-bromo-2,2-dimethyl-3-oxo-pyrrolo[2,3-b]pyridin-1-yl)methyl]pyridine-2-carbonitrile (172.45 mg, 482.79 μmol, 1 eq), CS₂CO₃ (393.25 mg, 1.21 mmol, 2.5 eq) and Pd(PPh₃)₂Cl₂ (33.89 mg, 48.28 μmol, 0.1 eq) in dioxane (4 mL) and H₂O (0.4 mL) was degassed and purged with N₂ for 3 times, and then the mixture was stirred at 80° C. for 2 h under N₂ atmosphere. The mixture was diluted with water 5 mL, and then extracted with Ethyl acetate 15 mL (5 mL*3). The combined organic layers were washed with sat. NaCl 10 mL, dried over Na₂SO₄, filtered and concentrated under reduced pressure to give a residue. The residue was purified by prep-TLC (SiO₂, Ethyl acetate:Methanol=10:1) to give compound tert-butyl N-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethoxy]ethyl]carbamate (150 mg, 231.93 μmol, 48.04% yield) as yellow oil.

### Data:

LCMS (ESI⁺): m/z 647.4 (M+H)

### 3. General Procedure for Preparation of 3-[[6-[2-[2-[2-[2-(2-aminoethoxy)ethoxy]ethoxy]ethylamino]pyrimidin-5-yl]-2,2-dimethyl-3-oxo-pyrrolo[2,3-b]pyridin-1-yl]methyl]pyridine-2-carbonitrile

A solution of tert-butyl N-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethoxy]ethyl]carbamate (150 mg, 231.93 μmol, 1 eq) in DCM (1.5 mL) and TFA (0.5 mL) was stirred at 15° C. for 1 hr. The DCM and TFA was removed under reduced pressure to give compound 3-[[6-[2-[2-[2-[2-(2-aminoethoxy)ethoxy]ethoxy]ethylamino]pyrimidin-5-yl]-2,2-dimethyl-3-oxo-pyrrolo[2,3-b]pyridin-1-yl]methyl]pyridine-2-carbonitrile (150 mg, crude, TFA) as yellow oil.

### Data:

LCMS (ESI⁺): m/z 547.3 (M+H)

### 4. General Procedure for Preparation of methyl (2S)-2-[[6-[3-[2-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethoxy]ethylamino]-2-oxo-ethoxy]phenoxy]pyridine-3-carbonyl]amino]-5,5-dimethyl-hexanoate

To a solution of 3-[[6-[2-[2-[2-[2-(2-aminoethoxy)ethoxy]ethoxy]ethylamino]pyrimidin-5-yl]-2,2-dimethyl-3-oxo-pyrrolo[2,3-b]pyridin-1-yl]methyl]pyridine-2-carbonitrile (150 mg, 227.05 μmol, 1 eq, TFA) and 2-[3-[[5-[[[(1S)-1-methoxycarbonyl-4,4-dimethyl-pentyl]carbamoyl]-2-pyridyl]oxy]phenoxy]acetic acid (126.81 mg, 227.05 μmol, 1 eq, TFA) in DMF (2 mL) was added DIEA (58.69 mg, 454.10 μmol, 79.10 μL, 2 eq) and HATU (129.50 mg, 340.58 μmol, 1.5 eq) at 0° C. The mixture was stirred at 15° C. for 1 hr. The mixture was diluted with water 5 mL, and then extracted with Ethyl acetate 15 mL (5 mL*3). The combined organic layers were washed with sat. NaCl 10 mL, dried over Na₂SO₄, filtered and concentrated under reduced pressure to give a residue. The residue was purified by prep-TLC (SiO₂, Ethyl acetate:Methanol=10:1) to give compound methyl (2S)-2-[[6-[3-[2-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethoxy]ethylamino]-2-oxo-ethoxy]phenoxy]pyridine-3-carbonyl]amino]-5,5-dimethyl-hexanoate (200 mg, 205.53 μmol, 90.52% yield) as yellow oil.

### Data:

LCMS (ESI⁺): m/z 973.4 (M+H)

### 5. General Procedure for Preparation of (2S)-2-[[6-[3-[2-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethoxy]ethylamino]-2-oxo-ethoxy]phenoxy]pyridine-3-carbonyl]amino]-5,5-dimethyl-hexanoic acid

The deprotection of methyl (2S)-2-[[6-[3-[2-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethoxy]ethylamino]-2-oxo-ethoxy]phenoxy]pyridine-3-carbonyl]amino]-5,5-dimethyl-hexanoate (200 mg, 205.53 μmol, 1 eq) was performed analogously to the description above of BF083 to give the compound of the title BF126 (105.7 mg, 108.69 μmol, 98.62% yield, 98.617% purity).

### Data:

LCMS (ESI⁺): m/z 959.5 (M+H)

¹H NMR (400 MHz, METHANOL-d4) δ=8.89 (s, 2H), 8.62 (d, J=2.9 Hz, 2H), 8.26-8.21 (m, 1H), 8.03 (dd, J=1.3, 8.1 Hz, 1H), 7.94 (d, J=8.1 Hz, 1H), 7.61 (dd, J=4.6, 8.2 Hz, 1H), 7.34 (t, J=8.2 Hz, 1H), 7.27 (d, J=8.1 Hz, 1H), 6.98 (d, J=8.7 Hz, 1H), 6.87 (dd, J=2.4, 8.3 Hz, 1H), 6.81-6.75 (m, 2H), 5.12 (s, 2H), 4.56-4.50 (m, 3H), 3.68-3.56 (m, 14H), 3.48 (q, J=5.0 Hz, 2H), 2.04-1.91 (m, 1H), 1.90-1.76 (m, 1H), 1.45-1.28 (m, 8H), 0.93 (s, 9H)

### BF139, BF143

### 1. General Procedure for Preparation of tert-butyl N-[2-[2-[2-[2-(5-bromopyrimidin-2-yl)oxyethoxy]ethoxy]ethoxy]ethyl]carbamate

To a stirred solution of tert-butyl N-[2-[2-[2-(2-hydroxyethoxy)ethoxy]ethoxy]ethyl]carbamate (1 g, 3.41 mmol, 1 eq) in THF (20 mL) was added NaH (409.02 mg, 10.23 mmol, 60% purity, 3 eq) at 0° C., then the reaction was stirred for 30 min at 0° C., then 5-bromo-2-chloro-pyrimidine (725.30 mg, 3.75 mmol, 1.1 eq) in THF (6 mL) was added at 0° C., then the reaction was stirred for 1.5 hr at 0° C. The mixture was quenched with saturated NH₄Cl solution (60 mL) at 0° C. and extracted with EtOAc 300 mL (100 mL*3). The combined organic layers were washed with brine 200 mL (100 mL*2), dried over anhydrous Na₂SO₄, filtered and concentrated under reduced pressure to give a residue. The residue was purified by flash silica gel chromatography (ISCO® 40 g SepaFlash® Silica Flash Column, Eluent of 0˜30% Ethyl acetate/Petroleum ether gradient @ 80 mL/min) to give compound tert-butyl N-[2-[2-[2-[2-(5-bromopyrimidin-2-yl)oxyethoxy]ethoxy]ethoxy]ethyl]carbamate (1.2 g, 2.66 mmol, 78.17% yield) as a yellow oil.

### Data:

LCMS (ESI+): m/z 450.2 (M+H)

### 2. General Procedure for Preparation of [2-[2-[2-[2-[2-(tert-butoxycarbonylamino)ethoxy]ethoxy]ethoxy]ethoxy]pyrimidin-5-yl]boronic acid

A mixture of tert-butyl N-[2-[2-[2-[2-(5-bromopyrimidin-2-yl)oxyethoxy]ethoxy]ethoxy]ethyl]carbamate (100.00 mg, 222.06 μmol, 1 eq), 4,4,5,5-tetramethyl-2-(4,4,5,5-tetramethyl-1,3,2-dioxaborolan-2-yl)-1,3,2-dioxaborolane (84.59 mg, 333.09 μmol, 1.5 eq), KOAc (65.38 mg, 666.19 μmol, 3 eq), Pd(dppf)Cl₂ (16.25 mg, 22.21 μmol, 0.1 eq) in dioxane (2 mL) was degassed and purged with N₂ for 3 times, and then the mixture was stirred at 90° C. for 12 hr under N₂ atmosphere. The reaction mixture of [2-[2-[2-[2-[2-(tert-butoxycarbonylamino)ethoxy]ethoxy]ethoxy]ethoxy]pyrimidin-5-yl]boronic acid (90 mg, crude) in dioxane (2 mL) was obtained as a black liquid, which was used to next step without purification.

### Data:

LCMS (ESI+): m/z 416.3 (M+H)

### 3. General Procedure for Preparation of tert-butyl N-[2-[2-[2-[2-[5-[1-[(2-cyano-3-pyridyl)methyl]-2,2-dimethyl-3-oxo-pyrrolo[2,3-b]pyridin-6-yl]pyrimidin-2-yl]oxyethoxy]ethoxy]ethoxy]ethyl]carbamate

A mixture of [2-[2-[2-[2-[2-(tert-butoxycarbonylamino)ethoxy]ethoxy]ethoxy]ethoxy]pyrimidin-5-yl]boronic acid (93 mg, 223.96 μmol, 1 eq), 3-[(6-bromo-2,2-dimethyl-3-oxo-pyrrolo[2,3-b]pyridin-1-yl)methyl]pyridine-2-carbonitrile (80.00 mg, 223.96 μmol, 1 eq), Cs₂CO₃ (182.43 mg, 559.91 μmol, 2.5 eq) and Pd(PPh₃)₂Cl₂ (15.72 mg, 22.40 μmol, 0.1 eq) in dioxane (2 mL) and H₂O (0.2 mL) was degassed and purged with N₂ for 3 times, and then the mixture was stirred at 85° C. for 2 hr under N₂ atmosphere. The mixture was diluted with water 5 mL, and then extracted with Ethyl acetate 15 mL (5 mL*3). The combined organic layers were washed with sat·NaCl 10 mL, dried over Na₂SO₄, filtered and concentrated under reduced pressure to give a residue. The residue was purified by prep-TLC (SiO₂, Ethyl acetate:Methanol=10:1) to give compound tert-butyl N-[2-[2-[2-[2-[5-[1-[(2-cyano-3-pyridyl)methyl]-2,2-dimethyl-3-oxo-pyrrolo[2,3-b]pyridin-6-yl]pyrimidin-2-yl]oxyethoxy]ethoxy]ethoxy]ethyl]carbamate (80 mg, 123.51 μmol, 55.15% yield) as a yellow oil.

### Data:

LCMS (ESI+): m/z 648.4 (M+H)

### 4. General Procedure for Preparation of 3-[[6-[2-[2-[2-[2-(2-aminoethoxy) ethoxy]ethoxy]ethoxy]pyrimidin-5-yl]-2,2-dimethyl-3-oxo-pyrrolo[2,3-b]pyridin-1-yl]methyl]pyridine-2-carbonitrile

A solution of tert-butyl N-[2-[2-[2-[2-[5-[1-[(2-cyano-3-pyridyl)methyl]-2,2-dimethyl-3-oxo-pyrrolo[2,3-b]pyridin-6-yl]pyrimidin-2-yl]oxyethoxy]ethoxy]ethoxy]ethyl]carbamate (160 mg, 247.02 μmol, 1 eq) in DCM (1.5 mL) and TFA (0.5 mL) was stirred at 15° C. for 1 hr. The DCM and TFA was removed under reduced pressure to give compound 3-[[6-[2-[2-[2-[2-(2-aminoethoxy)ethoxy]ethoxy]ethoxy]pyrimidin-5-yl]-2,2-dimethyl-3-oxo-pyrrolo[2,3-b]pyridin-1-yl]methyl]pyridine-2-carbonitrile (160 mg, crude, TFA) as a yellow oil.

### Data:

LCMS (ESI+): m/z 548.1 (M+H)

### 5. General Procedure for Preparation of methyl (2S)-2-[[6-[3-[2-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]oxyethoxy]ethoxy]ethoxy]ethylamino]-2-oxo-ethoxy]phenoxy]pyridine-3-carbonyl]amino]-5,5-dimethyl-hexanoate

To a solution of 3-[[6-[2-[2-[2-[2-(2-aminoethoxy)ethoxy]ethoxy]ethoxy]pyrimidin-5-yl]-2,2-dimethyl-3-oxo-pyrrolo[2,3-b]pyridin-1-yl]methyl]pyridine-2-carbonitrile (160 mg, 241.83 μmol, 1 eq, TFA), 2-[3-[[5-[[(1S)-1-methoxycarbonyl-4,4-dimethyl-pentyl]carbamoyl]-2-pyridyl]oxy]phenoxy]acetic acid (135.06 mg, 241.83 μmol, 1 eq, TFA) in DMF (3 mL) was added HATU (137.93 mg, 362.74 μmol, 1.5 eq) and DIEA (156.27 mg, 1.21 mmol, 210.61 μL, 5 eq) at 0° C. The mixture was stirred at 15° C. for 1 hr. The reaction mixture was diluted with H₂O 2 mL and extracted with DCM 6 mL (2 mL*3). The combined organic layers were washed with brine 10 mL (5 mL*2), dried over Na₂SO₄, filtered and concentrated under reduced pressure to give a residue. The residue was purified by prep-TLC (SiO₂, Ethyl acetate:MeOH=10:1) to give compound methyl (2S)-2-[[6-[3-[2-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]oxyethoxy]ethoxy]ethoxy]ethylamino]-2-oxo-ethoxy]phenoxy]pyridine-3-carbonyl]amino]-5,5-dimethyl-hexanoate (190 mg, 195.06 μmol, 80.66% yield) as a yellow oil

### Data:

LCMS (ESI+): m/z 974.6 (M+H)

### 6. General Procedure for Preparation of (2S)-2-[[6-[3-[2-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]oxyethoxy]ethoxy]ethoxy]ethylamino]-2-oxo-ethoxy]phenoxy]pyridine-3-carbonyl]amino]-5,5-dimethyl-hexanoic acid

The deprotection of methyl (2S)-2-[[6-[3-[2-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]oxyethoxy]ethoxy]ethoxy]ethylamino]-2-oxo-ethoxy]phenoxy]pyridine-3-carbonyl]amino]-5,5-dimethyl-hexanoate (190 mg, 195.06 μmol, 1 eq) was performed analogously to the description above of BF083 to give the compound of the title BF139 (74 mg, 76.66 μmol, 39.30% yield, 99.45% purity) as a yellow solid.

### Data:

LCMS (ESI+): m/z 960.2 (M+H)

¹H NMR (400 MHz, METHANOL-d₄) δ=9.08 (s, 2H), 8.62-8.58 (m, 2H), 8.22 (dd, J=2.4, 8.7 Hz, 1H), 8.04-7.97 (m, 2H), 7.60 (dd, J=4.7, 8.1 Hz, 1H), 7.37-7.29 (m, 2H), 6.97 (d, J=8.6 Hz, 1H), 6.86 (dd, J=2.3, 8.3 Hz, 1H), 6.79 (t, J=2.2 Hz, 1H), 6.75 (dd, J=1.9, 8.1 Hz, 1H), 5.12 (s, 2H), 4.58-4.54 (m, 2H), 4.53-4.47 (m, 3H), 3.86-3.81 (m, 2H), 3.68-3.61 (m, 4H), 3.60-3.53 (m, 6H), 3.44 (q, J=5.4 Hz, 2H), 2.00-1.91 (m, 1H), 1.86-1.75 (m, 1H), 1.40-1.31 (m, 8H), 0.91 (s, 9H)

The following compounds were prepared according to the general procedure.

The starting materials are either commercially available or may be prepared from commercially available reagents using conventional reactions well known in the art.

### BF143

### (S)-2-(6-(3-((20-((5-(1-((2-cyanopyridin-3-yl)methyl)-2,2-dimethyl-3-oxo-2,3-dihydro-1H-pyrrolo[2,3-b]pyridin-6-yl)pyrimidin-2-yl)oxy)-2-oxo-6,9, 12, 15, 18-pentaoxa-3-azaicosyl)oxy)phenoxy)nicotinamido)-5,5-dimethylhexanoic acid

LC/MS [M+1] 1048.5

¹H NMR (400 MHz, METHANOL-d4) δ=9.11 (s, 2H) 8.61 (m, 2H) 8.23 (dd, J=8.63, 2.50 Hz, 1H) 8.01 (m, 2H) 7.60 (dd, J=8.07, 4.69 Hz, 1H) 7.36 (m, 2H) 6.99 (d, J=8.63 Hz, 1H) 6.87 (dd, J=8.32, 2.19 Hz, 1H) 6.79 (m, 2H) 5.13 (s, 2H) 4.58 (dd, J=5.38, 4.00 Hz, 2H) 4.51 (m, 3H) 3.86 (m, 2H) 3.66 (m, 2H) 3.58 (m, 16H) 3.45 (q, J=5.21 Hz, 2H) 1.95 (m, 1H) 1.81 (m, 1H) 1.36 (m, 8H) 0.91 (s, 9H)

### BF075

### 1. General Procedure for Preparation of 5-azidopentan-1-ol

To a solution of 5-bromopentan-1-ol (1 g, 5.99 mmol, 1 eq) in DMF (15 mL) was added NaN₃ (450 mg, 6.92 mmol, 1.16 eq), then the reaction was stirred for 12 hr at 20° C. The reaction mixture was quenched by addition H₂O 30 mL, and then diluted with Na₂CO₃ 10 mL and extracted with Ethyl acetate 90 mL (30 mL*3). The combined organic layers were washed with brine 150 mL (75 mL*2), dried over Na₂SO₄, filtered and concentrated under reduced pressure to give a residue. The residue was not purified to give compound 5-azidopentan-1-ol (770 mg, crude) as a colorless oil.

### Data:

HNMR: ¹H NMR (400 MHz, CHLOROFORM-d) δ=3.65 (t, J=6.4 Hz, 2H), 3.28 (t, J=6.9 Hz, 2H), 1.68-1.54 (m, 4H), 1.49-1.40 (m, 2H)

### 2. General Procedure for Preparation of 2-(5-azidopentoxy)-5-bromo-pyrimidine

To a stirred solution of 5-azidopentan-1-ol (0.75 g, 5.81 mmol, 1.01 mL, 1 eq) in THF (17 mL) was added NaH (464.54 mg, 11.61 mmol, 60% purity, 2 eq) at 0° C., then the reaction was stirred for 30 min at 0° C., then 5-bromo-2-chloro-pyrimidine (1.24 g, 6.39 mmol, 1.1 eq) in THF (5 mL) was added at 0° C., then the reaction was stirred for 1.5 hr at 0° C. The mixture was quenched with saturated NH₄Cl solution (50 mL) at 0° C. and extracted with Ethyl acetate 150 mL (50 mL*3). The combined organic layers were dried over anhydrous Na₂SO₄, filtered and concentrated under reduced pressure to give a residue. The residue was purified by column chromatography (SiO₂, Petroleum ether/Ethyl acetate=1/0 to 10/1) to give compound 2-(5-azidopentoxy)-5-bromo-pyrimidine (1.5 g, 5.24 mmol, 90.28% yield) as a colourless oil.

### Data:

LCMS (ESI⁺): m/z 286.2 (M+H)

### 3. General Procedure for Preparation of [2-(5-azidopentoxy)pyrimidin-5-yl]boronic acid

A mixture of 2-(5-azidopentoxy)-5-bromo-pyrimidine (0.4 g, 1.40 mmol, 1 eq), 4,4,5,5-tetramethyl-2-(4,4,5,5-tetramethyl-1,3,2-dioxaborolan-2-yl)-1,3,2-dioxaborolane (532.50 mg, 2.10 mmol, 1.5 eq), KOAc (411.60 mg, 4.19 mmol, 3 eq), Pd(dppf)Cl₂ (102.29 mg, 139.80 umol, 0.1 eq) in dioxane (24 mL) was degassed and purged with N₂ for 3 times, and then the mixture was stirred at 95° C. for 2 hr under N₂ atmosphere. The residue was not purified to give compound [2-(5-azidopentoxy)pyrimidin-5-yl]boronic acid (1.05 g, crude) in dioxane (24 mL) as a black liquid.

### Data:

LCMS (ESI⁺): m/z 252.1 (M+H)

### 4. General Procedure for Preparation of 3-[[6-[2-(5-azidopentoxy)pyrimidin-5-yl]-2,2-dimethyl-3-oxo-pyrrolo[2,3-b]pyridin-1-yl]methyl]pyridine-2-carbonitrile

A mixture of [2-(5-azidopentoxy)pyrimidin-5-yl]boronic acid (350 mg, 1.39 mmol, 1 eq), 3-[(6-bromo-2,2-dimethyl-3-oxo-pyrrolo[2,3-b]pyridin-1-yl)methyl]pyridine-2-carbonitrile (298.80 mg, 836.49 umol, 0.6 eq), Cs₂CO₃ (1.14 g, 3.49 mmol, 2.5 eq) and Pd(PPh₃)₂Cl₂ (97.85 mg, 139.41 umol, 0.1 eq) in dioxane (10 mL) and H₂O (1 mL) was degassed and purged with N₂ for 3 times, and then the mixture was stirred at 85° C. for 2 hr under N₂ atmosphere. The insoluble was removed by filtration through celite, which was then washed with Ethyl acetate (10 ml). The mixture was diluted with water 5 mL, and then extracted with Ethyl acetate 15 mL (5 mL*3). The combined organic layers were washed with sat. NaCl 10 mL, dried over Na₂SO₄, filtered and concentrated under reduced pressure to give a residue. The residue was purified by flash silica gel chromatography (ISCO®; 20 g SepaFlash® Silica Flash Column, Eluent of 0˜35% Ethyl acetate/Petroleum ether gradient @ 50 mL/min) to give compound 3-[[6-[2-(5-azidopentoxy)pyrimidin-5-yl]-2,2-dimethyl-3-oxo-pyrrolo[2,3-b]pyridin-1-yl]methyl]pyridine-2-carbonitrile (550 mg, 1.14 mmol, 81.59% yield) as a yellow solid.

### Data:

LCMS (ESI⁺): m/z 484.2 (M+H)

### 5. General Procedure for Preparation of methyl (2S)-2-[[2-[3-[[5-[2-[2-[2-[2-[[1-[5-[5-[1-[(2-cyano-3-pyridyl)methyl]-2,2-dimethyl-3-oxo-pyrrolo[2,3-b]pyridin-6-yl]pyrimidin-2-yl]oxypentyl]triazol-4-yl]methoxy]ethoxy]ethoxy]ethoxy]ethylcarbamoyl]-2-pyridyl]oxy]phenoxy]acetyl]amino]-5,5-dimethyl-hexanoate

To a solution of 3-[[6-[2-(5-azidopentoxy)pyrimidin-5-yl]-2,2-dimethyl-3-oxo-pyrrolo[2,3-b]pyridin-1-yl]methyl]pyridine-2-carbonitrile (88.21 mg, 182.44 umol, 1.5 eq) and methyl (2S)-5,5-dimethyl-2-[[2-[3-[[5-[2-[2-[2-(2-prop-2-ynoxyethoxy)ethoxy]ethoxy]ethylcarbamoyl]-2-pyridyl]oxy]phenoxy]acetyl]amino]hexanoate (80.00 mg, 121.63 umol, 1 eq) in DMSO (1.5 mL) was added cuprous; acetonitrile; hexafluorophosphate (126.93 mg, 340.56 umol, 2.8 eq). The mixture was stirred at 20° C. for 1 hr. The mixture was diluted with water 3 mL, and then extracted with Ethyl acetate 15 mL (3 mL*3). The combined organic layers were washed with sat. NaCl 5 mL, dried over Na₂SO₄, filtered and concentrated under reduced pressure to give a residue. The residue was purified by prep-TLC (SiO₂, Ethyl acetate:Methanol=10:1) to give compound methyl (2S)-2-[[2-[3-[[5-[2-[2-[2-[2-[[1-[5-[5-[1-[(2-cyano-3-pyridyl)methyl]-2,2-dimethyl-3-oxo- pyrrolo[2,3-b]pyridin-6-yl]pyrimidin-2-yl]oxypentyl]triazol-4-yl]methoxy]ethoxy]ethoxy]ethoxy]ethylcarbamoyl]-2-pyridyl]oxy]phenoxy]acetyl]amino]-5,5-dimethyl-hexanoate (30 mg, 26.29 μmol, 21.61% yield) as a yellow oil.

### Data:

LCMS (ESI⁺): m/z 571.6 (M/2+H)

### 6. General Procedure for Preparation of methyl (2S)-2-[[2-[3-[[5-[2-[2-[2-[2-[[1-[5-[5-[1-[(2-cyano-3-pyridyl)methyl]-2,2-dimethyl-3-oxo-pyrrolo[2,3-b]pyridin-6-yl]pyrimidin-2-yl]oxypentyl]triazol-4-yl]methoxy]ethoxy]ethoxy]ethoxy]ethylcarbamoyl]-2-pyridyl]oxy]phenoxy]acetyl]amino]-5,5-dimethyl-hexanoic acid

The deprotection of methyl (2S)-2-[[2-[3-[[5-[2-[2-[2-[2-[[1-[5-[5-[1-[(2-cyano-3-pyridyl)methyl]-2,2-dimethyl-3-oxo-pyrrolo[2,3-b]pyridin-6-yl]pyrimidin-2-yl]oxypentyl]triazol-4-yl]methoxy]ethoxy]ethoxy]ethoxy]ethylcarbamoyl]-2-pyridyl]oxy]phenoxy]acetyl]amino]-5,5-dimethyl-hexanoate (30.00 mg, 26.29 umol, 1 eq) was performed analogously to the description above of BF083 to give the compound of the title BF075 (6.6 mg, 5.85 μmol, 22.27% yield, 100% purity) as a yellow solid.

### Data:

LCMS (ESI⁺): m/z 1127.5 (M+H)

¹H NMR (400 MHz, METHANOL-d4) δ ppm 9.12 (s, 2H) 8.64 (d, J=4.53 Hz, 1H) 8.60 (d, J=2.38 Hz, 1H) 8.22 (dd, J=8.58, 2.50 Hz, 1H) 8.01-8.07 (m, 2H) 7.98 (s, 1H) 7.63 (dd, J=8.11, 4.65 Hz, 1H) 7.33-7.43 (m, 2H) 6.99 (d, J=8.58 Hz, 1H) 6.91 (dd, J=8.23, 2.26 Hz, 1H) 6.84 (t, J=2.15 Hz, 1H) 6.79 (dd, J=7.81, 1.61 Hz, 1H) 5.15 (s, 2H) 4.60 (d, J=1.67 Hz, 5H) 4.42-4.46 (m, 4H) 3.59-3.68 (m, 14H) 3.54-3.58 (m, 2H) 1.97-2.06 (m, 2H) 1.82-1.94 (m, 3H) 1.67-1.80 (m, 1H) 1.45-1.54 (m, 2H) 1.38 (s, 6H)

### 1.20 (dd, J=9.48, 7.69 Hz, 2H) 0.87 (s, 9H)

### BF145

### 1. General Procedure for Preparation of tert-butyl N-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethyl]carbamate

To a solution of 3-[[2,2-dimethyl-6-[2-[(3R)-3-methylpiperazin-1-yl]pyrimidin-5-yl]-3-oxo-pyrrolo[2,3-b]pyridin-1-yl]methyl]pyridine-2-carbonitrile (150 mg, 263.83 umol, 1 eq, TFA), 2-[2-[2-[2-[2-(tert-butoxycarbonylamino)ethoxy]ethoxy]ethoxy]ethoxy]acetic acid (92.71 mg, 263.83 umol, 1 eq) and DIEA (102.29 mg, 791.49 umol, 137.86 uL, 3 eq) in DMF (2 mL) was added HATU (150.47 mg, 395.74 umol, 1.5 eq) at 0° C., then the reaction was stirred for 1 hr at 20° C. The reaction mixture was partitioned between water 5 mL and EtOAc 5 mL, the organic phase was separated, washed with brine 35 mL (5 mL*7), dried over [Na₂SO₄], filtered and concentrated under reduced pressure to give a residue. The residue was purified by prep-TLC to give compound tert-butyl N-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethyl]carbamate (150 mg, 190.38 umol, 72.16% yield) as a yellow oil.

### Data:

LCMS (ESI+): m/z 788.4 (M+H)

### 2. General Procedure for Preparation of 3-[[6-[2-[(3R)-4-[2-[2-[2-[2-(2-aminoethoxy)ethoxy]ethoxy]ethoxy]acetyl]-3-methyl-piperazin-1-yl]pyrimidin-5-yl]-2,2-dimethyl-3-oxo-pyrrolo[2,3-b]pyridin-1-yl]methyl]pyridine-2-carbonitrile

A solution of tert-butyl N-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethyl]carbamate (140 mg, 177.69 umol, 1 eq) in TFA (0.4 mL) and DCM (2 mL) was stirred at 20° C. for 1 hr. The mixture was concentrated under reduced pressure to give compound 3-[[6-[2-[(3R)-4-[2-[2-[2-[2-(2-aminoethoxy)ethoxy]ethoxy]ethoxy]acetyl]-3-methyl-piperazin-1-yl]pyrimidin-5-yl]-2,2-dimethyl-3-oxo-pyrrolo[2,3-b]pyridin-1-yl]methyl]pyridine-2-carbonitrile (140 mg, 174.61 umol, 98.27% yield, TFA) as a yellow oil.

### Data:

LCMS (ESI+): m/z 688.4 (M+H)

### 3. General Procedure for Preparation of methyl 2-[[4-[4-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethylcarbamoyl]phenyl]-6-methyl-2-pyridyl]carbamoyl]-5-(trifluoromethyl)benzoated

To a solution of 4-[2-[[2-methoxycarbonyl-4-(trifluoromethyl)benzoyl]amino]-6-methyl-4-pyridyl]benzoic acid (50 mg, 87.35 umol, 1 eq, TFA), 3-[[6-[2-[(3R)-4-[2-[2-[2-[2-(2-aminoethoxy)ethoxy]ethoxy]ethoxy]acetyl]-3-methyl-piperazin-1-yl]pyrimidin-5-yl]-2,2-dimethyl-3-oxo-pyrrolo[2,3-b]pyridin-1-yl]methyl]pyridine-2-carbonitrile (70.04 mg, 87.35 umol, 1 eq, TFA) in DMF (1 mL) was added CMPI (33.47 mg, 131.03 umol, 1.5 eq) and DIEA (33.87 mg, 262.05 umol, 45.64 uL, 3 eq) at 0° C., then the reaction was stirred for 1 hr at 25° C. The residue was diluted with water 2 mL and extracted with EtOAc 3 mL (1 mL*3). The combined organic layers were washed with brin 3 mL (1 mL*3), dried over [Na₂SO₄], filtered and concentrated under reduced pressure to give compound methyl 2-[[4-[4-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethylcarbamoyl]phenyl]-6-methyl-2-pyridyl]carbamoyl]-5-(trifluoromethyl)benzoate (100 mg, crude) as a yellow oil.

### Data:

LCMS (ESI⁺): m/z 1128.4 (M+H)

### 4. General Procedure for Preparation of 2-[[4-[4-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethylcarbamoyl]phenyl]-6-methyl-2-pyridyl]carbamoyl]-5-(trifluoromethyl)benzoic acid

The deprotection of methyl 2-[[4-[4-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethylcarbamoyl]phenyl]-6-methyl-2-pyridyl]carbamoyl]-5-(trifluoromethyl)benzoate (100 mg, 88.64 μmol, 1 eq) was performed analogously to the description above of BF083 to give the compound of the title BF145 (9 mg, 7.73 μmol, 8.72% yield, 95.736% purity) as a yellow solid.

### Data:

LCMS (ESI⁺): m/z 1114.4 (M+H)

¹H NMR (400 MHz, METHANOL-d4) δ=8.90 (s, 2H), 8.61 (dd, J=1.2, 4.6 Hz, 1H), 8.57 (br s, 1H), 8.20 (d, J=7.9 Hz, 1H), 8.03-7.98 (m, 1H), 7.96-7.86 (m, 5H), 7.80 (br d, J=8.1 Hz, 2H), 7.60 (dd, J=4.6, 8.1 Hz, 1H), 7.32 (s, 1H), 7.24 (d, J=8.1 Hz, 1H), 5.09 (s, 2H), 4.86-4.74 (m, 1H), 4.71-4.53 (m, 2H), 4.39-4.13 (m, 3H), 3.84-3.77 (m, 0.5H), 3.70-3.56 (m, 16.5H), 3.45-3.33 (m, 1H), 3.25-3.12 (m, 1H), 3.07-2.92 (m, 1H), 2.50 (s, 3H), 1.34 (s, 6H), 1.22-1.09 (m, 3H)

### BF146

### 1. General Procedure for Preparation of 5-(trifluoromethyl) isobenzofuran-1,3-dione

A mixture of 4-(trifluoromethyl) phthalic acid (10 g, 42.71 mmol, 1 eq) in SOCl₂ (100 mL) was stirred for 2 hr at 50° C. The mixture was concentrated to give 5-(trifluoromethyl) isobenzofuran-1,3-dione (9.2 g, crude) as a white solid, which was used to next step without purification.

### 2. General Procedure for Preparation of ethyl 4-(2-methoxyethoxymethyl)pyridine-2-carboxylate

A mixture of 4-chloro-6-methyl-pyridin-2-amine (10 g, 70.13 mmol, 1 eq), (4-tert-butoxycarbonylphenyl) boronic acid (15.57 g, 70.13 mmol, 1 eq), K₂CO₃ (2 M, 35.07 mL, 1 eq), Pd(OAc)₂ (787.27 mg, 3.51 mmol, 0.05 eq) and S-Phos (2.88 g, 7.01 mmol, 0.1 eq) in dioxane (20 mL) was degassed and purged with N₂ for 3 times, then the mixture was stirred at 100° C. for 1 hr under N₂ atmosphere. The reaction mixture was diluted with H₂O 30 mL and extracted with EA 90 mL (30 mL*3). The combined organic layers were washed with brine 60 mL (20 mL*3), dried over Na₂SO₄, filtered and concentrated under reduced pressure, and then purified by column chromatography (SiO₂, Petroleum ether/Ethyl acetate=1/0 to 1/1) to give tert-butyl 4-(2-amino-6-methyl-4-pyridyl)benzoate (7.5 g, 26.38 mmol, 37.61% yield) as a light yellow solid.

### Data:

LCMS (ESI⁺): m/z 285.1 (M+1)

### 3. General Procedure for Preparation of tert-butyl 4-[2-[1,3-dioxo-5-(trifluoromethyl) isoindolin-2-yl]-6-methyl-4-pyridyl]benzoate

A mixture of 5-(trifluoromethyl) isobenzofuran-1,3-dione (8 g, 37.02 mmol, 1 eq) and tert-butyl 4-(2-amino-6-methyl-4-pyridyl)benzoate (5.26 g, 18.51 mmol, 0.5 eq) in AcOH (120 mL) was stirred for 2 hr at 120° C. under N₂. The mixture was concentrated to give a residue, then the residue was purified by column chromatography (SiO₂, Petroleum ether/Ethyl acetate=1/0 to 10/1) to give tert-butyl 4-[2-[1,3-dioxo-5-(trifluoromethyl) isoindolin-2-yl]-6-methyl-4-pyridyl]benzoate (4.6 g, 9.53 mmol, 25.76% yield) as a yellow solid.

### 4. General Procedure for Preparation of 2-[[4-(4-tert-butoxycarbonylphenyl)-6-methyl-2-pyridyl]carbamoyl]-5-(trifluoromethyl)benzoic acid

To a solution of tert-butyl 4-[2-[1, 3-dioxo-5-(trifluoromethyl) isoindolin-2-yl]-6-methyl-4-pyridyl]benzoate (1.45 g, 3.01 mmol, 1 eq) in THF (7 mL) and H₂O (7 mL) was added LiOH·H₂O (378.36 mg, 9.02 mmol, 3 eq), then the mixture was stirred for 1 hr at 25° C. THF was removed and the aqueous layer was adjusted pH to 6-7 with 1M aq. HCl at 0° C., then the mixture was filtered and the filtrate was purified by prep-HPLC (HCl condition; column: Phenomenex luna C18 250*50 mm*10 um; mobile phase: [water (HCl)-ACN]; B %: 50%-70%, 10 min) to give 2-[[4-(4-tert-butoxycarbonylphenyl)-6-methyl-2-pyridyl]carbamoyl]-5-(trifluoromethyl)benzoic acid (200 mg, 399.63 umol, 13.30% yield) as a light yellow solid.

### Data:

LCMS (ESI⁺): m/z 501.2 (M+1)

¹H NMR (400 MHz, CHLOROFORM-d) δ=8.58 (s, 1H), 8.18-8.10 (m, 3H), 7.95-7.85 (m, 2H), 7.77 (d, J=8.3 Hz, 2H), 7.16 (s, 1H), 2.33 (s, 3H), 1.64 (s, 9H)

### 5. General Procedure for Preparation of methyl 2-[[4-(4-tert-butoxycarbonylphenyl)-6-methyl-2-pyridyl]carbamoyl]-5-(trifluoromethyl)benzoate

To a solution of 2-[[4-(4-tert-butoxycarbonylphenyl)-6-methyl-2-pyridyl]carbamoyl]-5-(trifluoromethyl)benzoic acid (400 mg, 799.26 umol, 1 eq) in MeOH (0.8 mL) and THF (3.2 mL) was added TMSCHN₂ (2 M, 239.78 uL, 1.2 eq) dropwise at 0° C., then the reaction was stirred for 0.5 hr at 0° C. and 11.5 hr at 25° C. The reaction mixture was diluted with H₂O 5 mL and extracted with EA 15 mL (5 mL*3). The combined organic layers were dried over anhydrous Na₂SO₄, filtered and concentrated under reduced pressure to give a residue, which was purified by prep-TLC (SiO₂, petroleum ether: ethyl acetate=5:1) to give methyl 2-[[4-(4-tert-butoxycarbonylphenyl)-6-methyl-2-pyridyl]carbamoyl]-5-(trifluoromethyl)benzoate (350 mg, 680.28 μmol, 85.11% yield) was obtained as a light yellow oil.

### Data:

LCMS (ESI+): m/z 515.1 (M+1)

### 6. General Procedure for Preparation of 4-[2-[[2-methoxycarbonyl-4-(trifluoromethyl)benzoyl]amino]-6-methyl-4-pyridyl]benzoic acid

A mixture of methyl 2-[[4-(4-tert-butoxycarbonylphenyl)-6-methyl-2-pyridyl]carbamoyl]-5-(trifluoromethyl)benzoate (130 mg, 252.68 umol, 1 eq) in DCM (1 mL) and TFA (0.2 mL) was stirred for 1 hr at 20° C. Concentrated under reduced pressure to give 4-[2-[[2-methoxycarbonyl-4-(trifluoromethyl)benzoyl]amino]-6-methyl-4-pyridyl]benzoic acid (0.2 g, crude, TFA) as a light yellow oil.

### Data:

LCMS (ESI+): m/z 459.1 (M+1)

### 7. General Procedure for Preparation of tert-butyl N-[12-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-12-oxo-dodecyl]carbamate

To a solution of 12-(tert-butoxycarbonylamino) dodecanoic acid (75.63 mg, 239.77 μmol, 1 eq) and 3-[[6-[2-[(3R)-4-(2-aminoacetyl)-3-methyl-piperazin-1-yl]pyrimidin-5-yl]-2,2-dimethyl-3-oxo-pyrrolo[2,3-b]pyridin-1-yl]methyl]pyridine-2-carbonitrile (150 mg, 239.77 μmol, 1 eq, TFA) in DMF (3 mL) was added HATU (136.75 mg, 359.65 μmol, 1.5 eq) and DIEA (92.96 mg, 719.31 μmol, 125.29 μL, 3 eq) at 0° C. The mixture was stirred at 25° C. for 2 hr. The mixture was diluted with water 10 mL, and then extracted with EtOAc 30 mL (10 mL*3). The combined organic layers were washed with brine 10 mL, dried over Na₂SO₄, filtered and concentrated under reduced pressure to give a residue, which was purified by prep-TLC (SiO2, Petroleum ether: Ethyl acetate=0:1) to give tert-butyl N-[12-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-12-oxo-dodecyl]carbamate (93 mg, 114.96 μmol, 47.94% yield) as a yellow solid

### Data:

LCMS (ESI+): m/z 809.4 (M+1)

### 8. General Procedure for Preparation of 12-amino-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]dodecanamide

A solution of tert-butyl N-[12-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-12-oxo-dodecyl]carbamate (60 mg, 74.16 umol, 1 eq) in DCM (1 mL) and TFA (0.2 mL) was stirred at 20° C. for 1 hr. Concentrated under reduced pressure to give 12-amino-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]dodecanamide (52 mg, 63.19 μmol, 85.20% yield, TFA) was obtained as a yellow oil, which was used to next step without purification.

### Data:

LCMS (ESI+): m/z 709.5 (M+1)

### 9. General Procedure for Preparation of methyl 2-[[4-[4-[[12-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-12-oxo-dodecyl]carbamoyl]phenyl]-6-methyl-2-pyridyl]carbamoyl]-5-(trifluoromethyl)benzoate

To a solution of 12-amino-N-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]dodecanamide (52 mg, 63.19 μmol, 1 eq, TFA), 4-[2-[[2-methoxycarbonyl-4-(trifluoromethyl)benzoyl]amino]-6-methyl-4-pyridyl]benzoic acid (28.97 mg, 50.60 μmol, 8.01e-1 eq, TFA) in DMF (1.5 mL) was added DIEA (24.50 mg, 189.57 μmol, 33.02 μL, 3 eq) and HATU (36.04 mg, 94.78 μmol, 1.5 eq) at 0° C. The mixture was stirred at 25° C. for 1 hr. The mixture was diluted with water 10 mL, and then extracted with EtOAc 30 mL (10 mL*3). The combined organic layers were washed with brine 10 mL, dried over Na₂SO₄, filtered and concentrated under reduced pressure to give methyl 2-[[4-[4-[[12-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-12-oxo-dodecyl]carbamoyl]phenyl]-6-methyl-2-pyridyl]carbamoyl]-5-(trifluoromethyl)benzoate (72 mg, crude) as a yellow oil, which was used to next step without purification.

### Data:

LCMS (ESI+): m/z 1149.5 (M+1)

### 10. General Procedure for Preparation of 2-[[4-[4-[[12-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-12-oxo-dodecyl]carbamoyl]phenyl]-6-methyl-2-pyridyl]carbamoyl]-5-(trifluoromethyl)benzoic acid

The deprotection of methyl 2-[[4-[4-[[12-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-12-oxo-dodecyl]carbamoyl]phenyl]-6-methyl-2-pyridyl]carbamoyl]-5-(trifluoromethyl)benzoate (50 mg, 43.51 μmol, 1 eq) was performed analogously to the description above of BF083 to give the compound of the title BF146 (7 mg, 6.03 μmol, 13.86% yield, 97.79% purity) as a yellow solid.

### Data:

LCMS (ESI+): m/z 1135.5 (M+1)

¹H NMR (400 MHz, METHANOL-d4) δ=8.94 (s, 2H), 8.62 (dd, J=1.4, 4.6 Hz, 1H), 8.59-8.54 (m, 1H), 8.37-8.33 (m, 1H), 8.17-8.12 (m, 1H), 8.04-8.01 (m, 1H), 7.94 (dd, J=4.4, 8.3 Hz, 3H), 7.91-7.87 (m, 1H), 7.84 (d, J=8.6 Hz, 2H), 7.64-7.58 (m, 1H), 7.35 (s, 1H), 7.29 (d, J=8.2 Hz, 1H), 5.11 (s, 2H), 4.94 (br d, J=6.6 Hz, 2H), 4.86-4.55 (m, 2H), 4.43-3.92 (m, 2H), 3.55-3.38 (m, 3H), 3.26-3.02 (m, 2H), 2.52 (s, 3H), 2.27 (t, J=7.5 Hz, 2H), 1.69-1.59 (m, 4H), 1.44-1.32 (m, 20H), 1.24-1.11 (m, 3H).

### BF104

### 1. General Procedure for Preparation of methyl 6-[3-[1-(9-hydroxynonyl)triazol-4-yl]phenoxy]pyridine-3-carboxylate

To a solution of the methyl 6-(3-ethynylphenoxy)pyridine-3-carboxylate (500 mg, 1.97 mmol, 1 eq) and d 9-azidononan-1-ol (365.77 mg, 1.97 mmol, 1 eq) in DMSO (10 mL) was added cuprous; acetonitrile; hexafluorophosphate (2.06 g, 5.53 mmol, 2.8 eq). The reaction was stirred for 12 hr at 25° C. The mixture was filtered and the filtrate was diluted with H₂O 20 mL and extracted with EA 60 mL (20 mL*3). The combined organic layers were dried over anhydrous Na₂SO₄, filtered and concentrated under reduced pressure to give a residue. The residue was purified by column chromatography (SiO₂, Petroleum ether/Ethyl acetate=1/0 to 0/1) to give methyl 6-[3-[1-(9-hydroxynonyl)triazol-4-yl]phenoxy]pyridine-3-carboxylate (720 mg, 1.64 mmol, 83.16% yield) as a white solid.

### Data:

LCMS (ESI⁺): m/z 439.2 (M+H)

### 2. General Procedure for Preparation of methyl 6-[3-[1-[9-(p-tolylsulfonyloxy) nonyl]triazol-4-yl]phenoxy]pyridine-3-carboxylate

To a solution of methyl 6-[3-[1-(9-hydroxynonyl)triazol-4-yl]phenoxy]pyridine-3-carboxylate (720 mg, 1.64 mmol, 1 eq) in DCM (15 mL) was added TEA (332.29 mg, 3.28 mmol, 457.07 uL, 2 eq) and TosCl (626.05 mg, 3.28 mmol, 2 eq) at 0° C., then the reaction was stirred for 12 hr at 25° C. The reaction mixture was diluted with H₂O 20 mL and extracted with EA 60 mL (20 mL*3). The combined organic layers were dried over anhydrous Na₂SO₄, filtered and concentrated under reduced pressure to give a residue. The residue was purified by column chromatography (SiO₂, Petroleum ether/Ethyl acetate=1/0 to 1/1) to give methyl 6-[3-[1-[9-(p-tolylsulfonyloxy) nonyl]triazol-4-yl]phenoxy]pyridine-3-carboxylate (600 mg, 1.01 mmol, 61.65% yield) as a white solid.

### Data:

LCMS (ESI⁺): m/z 593.3 (M+H)

### 3. General Procedure for Preparation of methyl 6-[3-[1-(9-azidononyl)triazol-4-yl]phenoxy]pyridine-3-carboxylate

To a solution of methyl 6-[3-[1-[9-(p-tolylsulfonyloxy) nonyl]triazol-4-yl]phenoxy]pyridine-3-carboxylate (600 mg, 1.01 mmol, 1 eq) in DMF (70 mL) was added NaN₃ (90 mg, 1.38 mmol, 1.37 eq), then the reaction was stirred for 3 hr at 60° C. TLC showed R¹ was consumed and one new spot with lower polarity was detected. The reaction mixture was quenched by addition H₂O 20 mL, and then diluted with Na₂CO_{3 10} mL and extracted with EtOAc 90 mL (30 mL*3). The combined organic layers were washed with brine 180 mL (90 mL*2), dried over Na₂SO₄, filtered and concentrated under reduced pressure to give methyl 6-[3-[1-(9-azidononyl)triazol-4-yl]phenoxy]pyridine-3-carboxylate (940 mg, crude) as a white solid.

### Data:

LCMS (ESI⁺): m/z 464.2 (M+H)

### 4. General Procedure for Preparation of 2-[1-[9-[4-[3-[(5-methoxycarbonyl-2-pyridyl)oxy]phenyl]triazol-1-yl]nonyl]triazol-4-yl]acetic acid

To a solution of methyl 6-[3-[1-(9-azidononyl)triazol-4-yl]phenoxy]pyridine-3-carboxylate (551.34 mg, 1.19 mmol, 1 eq) and but-3-ynoic acid (100 mg, 1.19 mmol, 1 eq) in t-BuOH (2 mL) and H₂O (2 mL) was added sodium; (2R)-2-[(1S)-1,2-dihydroxyethyl]-4-hydroxy-5-oxo-2H-furan-3-olate (235.64 mg, 1.19 mmol, 1 eq) and copper; sulfate (94.92 mg, 594.72 umol, 91.27 uL, 0.5 eq) then the reaction was stirred for 12 hr at 50° C. The reaction mixture was diluted with H₂O 5 mL, acidified with HCl (1M) till pH=2, filtered and the filter was extracted with EA 15 mL (5 mL*3). The combined organic layers were dried over anhydrous Na₂SO₄, filtered and concentrated under reduced pressure to give 2-[1-[9-[4-[3-[(5-methoxycarbonyl-2-pyridyl)oxy]phenyl]triazol-1-yl]nonyl]triazol-4-yl]acetic acid (300 mg, crude) was obtained as a yellow oil.

### Data:

LCMS (ESI⁺): m/z 548.2 (M+H)

### 5. General Procedure for Preparation of methyl 6-[3-[1-[9-[4-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-1-yl]nonyl]triazol-4-yl]phenoxy]pyridine-3-carboxylate

To a solution of 2-[1-[9-[4-[3-[(5-methoxycarbonyl-2-pyridyl)oxy]phenyl]triazol-1-yl]nonyl]triazol-4-yl]acetic acid (90 mg, 164.35 umol, 1 eq) and 3-[[2,2-dimethyl-6-[2-[(3R)-3-methylpiperazin-1-yl]pyrimidin-5-yl]-3-oxo-pyrrolo[2,3-b]pyridin-1-yl]methyl]pyridine-2-carbonitrile (93.44 mg, 164.35 umol, 1 eq, TFA) in DMF (2 mL) was added DIEA (106.20 mg, 821.76 umol, 143.13 uL, 5 eq) and HATU (124.98 mg, 328.70 umol, 2 eq) at 0° C., then the reaction was stirred for 1 hr at 25° C. The reaction mixture was diluted with H₂O 2 mL and extracted with EA 6 mL (2 mL*3). The combined organic layers were washed with brine (5 mL*3), dried over anhydrous Na₂SO₄, filtered and concentrated under reduced pressure to give a residue. The residue was purified by prep-TLC (SiO₂, ethyl acetate:MeOH=10:1) to give methyl 6-[3-[1-[9-[4-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-1-yl]nonyl]triazol-4-yl]phenoxy]pyridine-3-carboxylate (100 mg, 101.61 umol, 61.83% yield) as a yellow oil.

### Data:

LCMS (ESI⁺): m/z 984.4 (M+H)

### 6. General Procedure for Preparation of 6-[3-[1-[9-[4-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-1-yl]nonyl]triazol-4-yl]phenoxy]pyridine-3-carboxylic acid

To a solution of methyl 6-[3-[1-[9-[4-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-1-yl]nonyl]triazol-4-yl]phenoxy]pyridine-3-carboxylate (100 mg, 101.61 umol, 1 eq) in THF (1 mL) and H₂O (0.5 mL) was added LiOH·H₂O (8.53 mg, 203.23 umol, 2 eq), then the reaction was stirred for 1 hr at 25° C. THF was removed under reduced pressure. The aqueous layer was adjusted pH to 3-4 with 1M aq·HCl, and extracted with EA 9 mL (3 mL*3). The combined organic layers were dried over anhydrous Na₂SO₄, filtered and concentrated under reduced pressure to give 6-[3-[1-[9-[4-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-1-yl]nonyl]triazol-4-yl]phenoxy]pyridine-3-carboxylic acid (98 mg, crude, HCl) as yellow oil.

### Data:

LCMS (ESI⁺): m/z 970.4 (M+H)

### 7. General Procedure for Preparation of (2,5-dioxopyrrolidin-1-yl) 6-[3-[1-[9-[4-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-1-yl]nonyl]triazol-4-yl]phenoxy]pyridine-3-carboxylate

To a mixture of 6-[3-[1-[9-[4-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-1-yl]nonyl]triazol-4-yl]phenoxy]pyridine-3-carboxylic acid (95 mg, 94.38 umol, 1 eq, HCl) in DMF (2 mL) was added HOSu (16.29 mg, 141.57 umol, 1.5 eq) and EDCI (36.19 mg, 188.76 umol, 2 eq), then the mixture was stirred for 0.5 hr at 25° C. The reaction mixture was diluted with H₂O 2 mL and extracted with EA 9 mL (3 mL*3). The combined organic layers were washed with brine (10 mL*3), dried over anhydrous Na₂SO₄, filtered and concentrated under reduced pressure to (2,5-dioxopyrrolidin-1-yl)

### 6-[3-[1-[9-[4-[2-[(2R)-4-[5-[1-[[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-1-yl]nonyl]triazol-4-yl]phenoxy]pyridine-3-carboxylate (100 mg, crude) as yellow oil

### Data:

LCMS (ESI⁺): m/z 1067.5 (M+H)

### 8. General Procedure for Preparation of (2S)-2-[[6-[3-[1-[9-[4-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-1-yl]nonyl]triazol-4-yl]phenoxy]pyridine-3-carbonyl]amino]-5,5-dimethyl-hexanoic acid

To a solution of (2S)-2-amino-5,5-dimethyl-hexanoic acid (14.92 mg, 93.71 umol, 1 eq) and (2,5-dioxopyrrolidin-1-yl) 6-[3-[1-[9-[4-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-1-yl]nonyl]triazol-4-yl]phenoxy]pyridine-3-carboxylate (100 mg, 93.71 umol, 1 eq) in DMF (0.6 mL), DCM (0.1 mL) and H₂O (0.3 mL) was added DIEA (24.22 mg, 187.41 umol, 32.64 uL, 2 eq). The reaction was degassed and purged with N₂ for 3 times, and then stirred at 20° C. for 1 hr under N₂ atmosphere. THF was removed under reduced pressure. The aqueous layer was adjusted pH to 3-4 with 1M aq. FA. The mixture was purified by prep-HPLC (FA condition; column: Phenomenex Luna C18 75*30 mm*3 um; mobile phase: [water (FA)-ACN]; B %: 50%-90%, 8 min) to give (2S)-2-[[6-[3-[1-[9-[4-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-1-yl]nonyl]triazol-4-yl]phenoxy]pyridine-3-carbonyl]amino]-5,5-dimethyl-hexanoic acid (20 mg, 16.96 μmol, 18.10% yield, 94.230% purity) as a light yellow solid.

### Data:

LCMS (ESI⁺): m/z 1111.6 (M+H)

¹H NMR (400 MHz, METHANOL-d4) δ=8.94 (s, 2H), 8.64-8.62 (m, 2H), 8.35 (s, 1H), 8.28 (dd, J=2.5, 8.6 Hz, 1H), 8.03 (d, J=7.9 Hz, 1H), 7.95 (d, J=8.0 Hz, 1H), 7.87 (s, 1H), 7.74-7.69 (m, 1H), 7.66-7.60 (m, 2H), 7.52 (t, J=7.9 Hz, 1H), 7.30 (d, J=8.0 Hz, 1H), 7.17-7.12 (m, 1H), 7.08 (d, J=8.7 Hz, 1H), 5.12 (s, 2H), 4.67-4.62 (m, 3H), 4.53 (br dd, J=5.0, 8.7 Hz, 2H), 4.45-4.37 (m, 4H), 4.06-3.86 (m, 3H), 3.18-3.01 (m, 2H), 2.01-1.83 (m, 6H), 1.38-1.24 (m, 19H), 1.14 (br d, J=6.6 Hz, 2H), 0.93 (s, 9H)

### BF096

### 1. General Procedure for Preparation of methyl (2S)-5,5-dimethyl-2-[[2-[3-[[5-[[1-[2-[2-(2-prop-2-ynoxyethoxy)ethoxy]acetyl]-4-piperidyl]oxy]-2-pyridyl]oxy]phenoxy]acetyl]amino]hexanoate

To a solution of methyl (2S)-5,5-dimethyl-2-[[2-[3-[[5-(4-piperidyloxy)-2-pyridyl]oxy]phenoxy]acetyl]amino]hexanoate (250 mg, 500.40 μmol, 1 eq) and 2-[2-(2-prop-2-ynoxyethoxy)ethoxy]acetic acid (101.18 mg, 500.40 μmol, 1 eq) in DMF (4 mL) was added DIEA (129.35 mg, 1.00 mmol, 174.32 μL, 2 eq) and HATU (285.40 mg, 750.60 μmol, 185.93 μL, 1.5 eq) at 0° C. The mixture was stirred at 15° C. for 0.5 hr. The mixture was diluted with water 5 mL, and extracted with ethyl acetate 15 mL (5 mL*3). The combined organic layers were washed with brine 10 mL, dried over Na₂SO₄, filtered and concentrated under reduced pressure to give a residue. The residue was purified by prep-TLC (SiO₂, Petroleum ether: Ethyl acetate=1:1) to give compound methyl (2S)-5,5-dimethyl-2-[[2-[3-[[5-[[1-[2-[2-(2-prop-2-ynoxyethoxy)ethoxy]acetyl]-4-piperidyl]oxy]-2-pyridyl]oxy]phenoxy]acetyl]amino]hexanoate (270 mg, 394.86 μmol, 78.91% yield) as a yellow oil.

### Data:

LCMS (ESI⁺): m/z 684.5 (M+H)

### 2. General Procedure for Preparation of methyl (2S)-2-[[2-[3-[[5-[[1-[2-[2-[2-[[1-[2-[(2R)-4-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]-2-methyl-piperazin-1-yl]-2-oxo-ethyl]triazol-4-yl]methoxy]ethoxy]ethoxy]acetyl]-4-piperidyl]oxy]-2-pyridyl]oxy]phenoxy]acetyl]amino]-5,5-dimethyl-hexanoate

To a solution of methyl (2S)-5,5-dimethyl-2-[[2-[3-[[5-[[1-[2-[2-(2-prop-2-ynoxyethoxy)ethoxy]acetyl]-4-piperidyl]oxy]-2-pyridyl]oxy]phenoxy]acetyl]amino]hexanoate (60.85 mg, 88.99 μmol, 1.1 eq) and 3-[(1R)-1-[[6-[2-[(3R)-4-(2-azidoacetyl)-3-methyl-piperazin-1-yl]pyrimidin-5-yl]-3-chloro-7-fluoro-2-methyl-1,5-naphthyridin-4-yl]amino]ethyl]-4-fluoro-benzonitrile (50 mg, 80.90 μmol, 1 eq) in t-BuOH (0.5 mL) and H₂O (0.5 mL) was added CuSO₄·5H₂O (10.14 mg, 40.45 μmol, 6.21 μL, 0.5 eq) and sodium; (2R)-2-[(1S)-1,2-dihydroxyethyl]-4-hydroxy-5-oxo-2H-furan-3-olate (16.03 mg, 80.90 μmol, 1 eq). The mixture was stirred at 50° C. for 0.5 hr. The mixture was diluted with water 3 mL, and then extracted with Ethyl acetate 9 mL (3 mL*3). The combined organic layers were washed with brine 10 mL, dried over Na₂SO₄, filtered and concentrated under reduced pressure to give a residue. The residue was purified by prep-TLC (SiO₂, Ethyl acetate:Methanol=5:1) to give compound methyl (2S)-2-[[2-[3-[[5-[[1-[2-[2-[2-[[1-[2-[(2R)-4-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]-2-methyl-piperazin-1-yl]-2-oxo-ethyl]triazol-4-yl]methoxy]ethoxy]ethoxy]acetyl]-4-piperidyl]oxy]-2-pyridyl]oxy]phenoxy]acetyl]amino]-5,5-dimethyl-hexanoate (80 mg, 61.45 μmol, 75.96% yield) as a yellow solid.

### Data:

LCMS (ESI⁺): m/z 1301.7 (M+H)

### 3. General Procedure for Preparation of (2S)-2-[[2-[3-[[5-[[1-[2-[2-[2-[[1-[2-[(2R)-4-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]-2-methyl-piperazin-1-yl]-2-oxo-ethyl]triazol-4-yl]methoxy]ethoxy]ethoxy]acetyl]-4-piperidyl]oxy]-2-pyridyl]oxy]phenoxy]acetyl]amino]-5,5-dimethyl-hexanoic acid

To a solution of methyl (2S)-2-[[2-[3-[[5-[[1-[2-[2-[2-[[1-[2-[(2R)-4-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]-2-methyl-piperazin-1-yl]-2-oxo-ethyl]triazol-4-yl]methoxy]ethoxy]ethoxy]acetyl]-4-piperidyl]oxy]-2-pyridyl]oxy]phenoxy]acetyl]amino]-5,5-dimethyl-hexanoate (80 mg, 61.45 μmol, 1 eq) in THF (0.5 mL) and H₂O (0.5 mL) was added LiOH·H₂O (5.16 mg, 122.90 μmol, 2 eq). The mixture was stirred at 15° C. for 0.5 hr. The THF was removed under reduced pressure and FA was added to the residue until pH=2. The mixture was filtered and the filtrate was purified by prep-HPLC (FA condition; column: Phenomenex luna C18 100*40 mm*3 um; mobile phase: [H₂O (0.2% FA)-ACN]; gradient: 50%-90% B over 8.0 min) to give compound (2S)-2-[[2-[3-[[5-[[1-[2-[2-[2-[[1-[2-[(2R)-4-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]-2-methyl-piperazin-1-yl]-2-oxo-ethyl]triazol-4-yl]methoxy]ethoxy]ethoxy]acetyl]-4-piperidyl]oxy]-2-pyridyl]oxy]phenoxy]acetyl]amino]-5,5-dimethyl-hexanoic acid (31.8 mg, 24.25 μmol, 39.46% yield, 98.203% purity) as a yellow solid.

### Data:

LCMS (ESI⁺): m/z 1287.5 (M+H)

¹H NMR (400 MHz, METHANOL-d4) δ ppm 8.86 (s, 2H) 8.00 (br d, J=5.87 Hz, 1H) 7.85-7.94 (m, 2H) 7.81 (dd, J=6.97, 1.83 Hz, 1H) 7.65 (ddd, J=8.44, 4.71, 2.02 Hz, 1H) 7.51 (dd, J=8.80, 3.06 Hz, 1H) 7.17-7.33 (m, 2H) 6.91 (d, J=8.93 Hz, 1H) 6.78 (dd, J=8.19, 2.20 Hz, 1H) 6.71 (t, J=2.14 Hz, 1H) 6.59-6.68 (m, 2H) 5.54-5.73 (m, 1H) 5.37-5.50 (m, 1H) 4.70-4.86 (m, 3H) 4.67 (s, 2H) 4.57-4.63 (m, 1H) 4.56 (s, 2H) 4.34-4.47 (m, 2H) 4.27 (d, J=3.30 Hz, 2H) 3.74-3.96 (m, 2H) 3.63-3.74 (m, 9H) 3.36-3.63 (m, 4H) 3.07-3.21 (m, 1H) 2.75 (s, 3H) 1.83-2.07 (m, 3H) 1.64-1.81 (m, 6H) 1.33-1.39 (m, 1H) 1.12-1.28 (m, 4H) 0.85 (s, 9H)

### BF147

### 1. General Procedure for Preparation of 2-[2-[2-[2-[2-(2-prop-2-ynoxyethoxy)ethoxy]ethoxy]ethoxy]ethoxy]ethyl 4-methylbenzenesulfonate

To a solution of 2-[2-[2-[2-[2-(2-prop-2-ynoxyethoxy)ethoxy]ethoxy]ethoxy]ethoxy]ethanol (0.5 g, 1.56 mmol, 1 eq) in DCM (10 mL) was added TEA (315.84 mg, 3.12 mmol, 434.45 UL, 2 eq), 4-methylbenzenesulfonyl chloride (595.07 mg, 3.12 mmol, 2 eq) and DMAP (19.07 mg, 156.07 μmol, 0.1 eq) at 0° C., then the reaction was stirred for 12 hr at 20° C. The reaction mixture was partitioned between water 20 mL and EtOAc 20 mL. The organic phase was separated, washed with brine 30 mL (10 mL*3), dried over Na₂SO₄, filtered and concentrated under reduced pressure to give a residue. The residue was purified by prep-TLC to give compound 2-[2-[2-[2-[2-(2-prop-2-ynoxyethoxy)ethoxy]ethoxy]ethoxy]ethoxy]ethyl 4-methylbenzenesulfonate (0.5 g, 1.05 mmol, 67.51% yield) as a light yellow oil.

### Data:

¹H NMR (400 MHz, CHLOROFORM-d) δ=7.80 (d, J=8.3 Hz, 2H), 7.34 (d, J=8.1 Hz, 2H), 4.20 (d, J=2.3 Hz, 2H), 3.72-3.60 (m, 20H), 3.58 (s, 4H), 2.45 (s, 3H), 2.43 (t, J=2.4 Hz, 1H)

### 2. General Procedure for Preparation of 3-[2-[2-[2-[2-[2-(2-prop-2-ynoxyethoxy)ethoxy]ethoxy]ethoxy]ethoxy]ethoxy]benzaldehyde

To a solution of 2-[2-[2-[2-[2-(2-prop-2-ynoxyethoxy)ethoxy]ethoxy]ethoxy]ethoxy]ethyl 4-methylbenzenesulfonate (0.4 g, 842.88 μmol, 1 eq), 3-hydroxybenzaldehyde (102.93 mg, 842.88 μmol, 1 eq) in ACN (5 mL) was added Cs₂CO₃ (411.94 mg, 1.26 mmol, 1.5 eq) at 0° C., then the reaction was stirred for 1 hr at 80° C. The reaction mixture was partitioned between water 20 mL and EtOAc 20 mL. The organic phase washed with brine 30 mL (10 mL*3), dried over Na₂SO₄, filtered and concentrated under reduced pressure to give a residue. The residue was purified by prep-TLC to give compound 3-[2-[2-[2-[2-[2-(2-prop-2-ynoxyethoxy)ethoxy]ethoxy]ethoxy]ethoxy]ethoxy]benzaldehyde (160 mg, 376.93 μmol, 44.72% yield) as a light yellow solid.

### Data:

¹H NMR (400 MHz, CHLOROFORM-d) δ=9.97 (s, 1H), 7.47-7.46 (m, 1H), 7.41 (d, J=1.8 Hz, 1H), 7.27 (s, 1H), 7.21 (td, J=2.4, 7.2 Hz, 1H), 4.22-4.18 (m, 5H), 3.90-3.87 (m, 2H), 3.76-3.66 (m, 19H), 2.43 (t, J=2.3 Hz, 1H)

### 3. General Procedure for Preparation of methyl (2S)-5,5-dimethyl-2-[[3-[2-[2-[2-[2-[2-(2-prop-2-ynoxyethoxy)ethoxy]ethoxy]ethoxy]ethoxy]ethoxy]phenyl]methylamino]hexanoate

TEA (35.76 mg, 353.37 μmol, 49.19 μL, 1 eq) was added to a solution of methyl (2S)-2-amino-5,5-dimethyl-hexanoate (61.22 mg, 353.37 μmol, 1 eq) in EtOH (2 mL) until pH=8˜9, the reaction was stirred for 15 min at 0° C., then 3-[2-[2-[2-[2-[2-(2-prop-2-ynoxyethoxy)ethoxy]ethoxy]ethoxy]ethoxy]ethoxy]benzaldehyde (150 mg, 353.37 μmol, 1 eq) was added, and AcOH (42.44 mg, 706.74 μmol, 40.46 μL, 2 eq) was added to adjust pH=5, the reaction was stirred for 15 min at 25° C. Then NaBH3CN (44.41 mg, 706.74 μmol, 2 eq) in EtOH (2 mL) was added, and the reaction was stirred for 0.5 hr at 25° C. The reaction mixture was partitioned between water 15 mL and EtOAc 15 mL. The organic phase was washed with brine 30 mL (10 mL*3), dried over Na₂SO₄, filtered and concentrated under reduced pressure to give a residue. The residue was purified by flash silica gel chromatography (ISCO®; 20 g SepaFlash® Silica Flash Column, Eluent of 0˜35% Ethylacetate/Petroleum ether gradient @ 50 mL/min) to give compound methyl (2S)-5,5-dimethyl-2-[[3-[2-[2-[2-[2-[2-(2-prop-2-ynoxyethoxy)ethoxy]ethoxy]ethoxy]ethoxy]ethoxy]phenyl]methylamino]hexanoate (70 mg, 111.50 μmol, 31.55% yield, 92.662% purity) as a yellow oil.

### Data:

LCMS (ESI⁺): m/z 582.6 (M+H)

### 4. General Procedure for Preparation of methyl (2S)-2-[[3-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethoxy]ethoxy]ethoxy]phenyl]methylamino]-5,5-dimethyl-hexanoate

To a solution of the methyl (2S)-5,5-dimethyl-2-[[3-[2-[2-[2-[2-[2-(2-prop-2-ynoxyethoxy)ethoxy]ethoxy]ethoxy]ethoxy]ethoxy]phenyl]methylamino]hexanoate (50 mg, 85.95 μmol, 1 eq) and 3-[(1R)-1-[[6-[2-(5-azidopentoxy)pyrimidin-5-yl]-3-chloro-7-fluoro-2-methyl-1,5-naphthyridin-4-yl]amino]ethyl]-4-fluoro-benzonitrile (48.47 mg, 85.95 μmol, 1 eq) in t-BuOH (0.5 mL) and H₂O (0.5 mL) was added CuSO₄·5H₂O (21.54 mg, 85.95 μmol, 13.19 μL, 1 eq), sodium; (2R)-2-[(1S)-1,2-dihydroxyethyl]-4-hydroxy-5-oxo-2H-furan-3-olate (17.03 mg, 85.95 μmol, 1 eq). The reaction was stirred for 0.5 hr at 50° C. The crude product was filtered and the filtrate was purified by reversed-phase HPLC flash C18 gel chromatography (ISCO; 80 g SepaFlash C18 Flash Column, eluent of 0-65% MeCN/H₂O 70 ml/min) to give compound methyl (2S)-2-[[3-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethoxy]ethoxy]ethoxy]phenyl]methylamino]-5,5-dimethyl-hexanoate (60 mg, 52.37 μmol, 60.93% yield) as a yellow oil.

### Data:

LCMS (ESI⁺): m/z 1145.7 (M+H)

### 5. General Procedure for Preparation of (2S)-2-[[3-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethoxy]ethoxy]ethoxy]phenyl]methylamino]-5,5-dimethyl-hexanoic acid

To a solution of methyl (2S)-2-[[3-[2-[2-[2-[2-[2-[2-[[1-[5-[5-[7-chloro-8-[[(1R)-1-(5-cyano- 2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethoxy]ethoxy]ethoxy]phenyl]methylamino]-5,5-dimethyl-hexanoate (50 mg, 43.64 μmol, 1 eq) in H₂O (0.5 mL) and THF (0.5 mL) was added LiOH·H₂O (3.66 mg, 87.28 μmol, 2 eq). The mixture was stirred at 25° C. for 12 hr. The mixture was concentrated under reduced pressure to give a residue. The crude product was filtered and the filtrate was purified by reversed-phase HPLC (column: C18 20-35 um 100 A 40 g; mobile phase: [water-ACN]; B %: 0%-63% @ 75 mL/min) to give compound (2S)-2-[[3-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethoxy]ethoxy]ethoxy]phenyl]methylamino]-5,5-dimethyl-hexanoic acid (36.9 mg, 32.44 μmol, 74.33% yield, 99.487% purity) as a white solid.

### Data:

LCMS (ESI⁺): m/z 1131.5 (M+H)

¹H NMR (400 MHz, METHANOL-d₄) δ=9.01 (d, J=1.2 Hz, 2H), 8.03 (s, 1H), 7.93 (d, J=11.6 Hz, 1H), 7.80 (dd, J=2.1, 7.0 Hz, 1H), 7.64 (ddd, J=2.1, 4.7, 8.5 Hz, 1H), 7.38-7.30 (m, 1H), 7.20 (dd, J=8.6, 10.3 Hz, 1H), 7.10 (d, J=1.9 Hz, 1H), 7.06 (d, J=7.6 Hz, 1H), 7.00 (dd, J=2.1, 8.1 Hz, 1H), 6.47 (q, J=6.7 Hz, 1H), 4.64 (s, 2H), 4.55-4.46 (m, 4H), 4.23-4.07 (m, 4H), 3.84 (dd, J=3.8, 5.4 Hz, 2H), 3.71-3.60 (m, 20H), 3.50 (dd, J=5.4, 6.6 Hz, 1H), 2.72 (s, 3H), 2.05 (quin, J=7.4 Hz, 2H), 1.97-1.79 (m, 4H), 1.74 (d, J=6.9 Hz, 3H), 1.62-1.51 (m, 2H), 1.45-1.26 (m, 2H), 0.91 (s, 9H)

### BF148

### 1. General Procedure for Preparation of tert-butyl 2-(3-hydroxyphenoxy)acetate

To a solution of tert-butyl 2-bromoacetate (8 g, 41.01 mmol, 6.06 mL, 1 eq), benzene-1,3-diol (4.52 g, 41.01 mmol, 6.84 mL, 1 eq) in ACN (500 mL) was added K₂CO₃ (5.67 g, 41.01 mmol, 1 eq). The mixture was stirred at 80° C. for 3 hr. The mixture was diluted with water 200 mL, and then extracted with EtOAc 1000 mL (250 mL*4). The combined organic layers were washed with sat·NaCl 100 mL, dried over Na₂SO₄, filtered and concentrated under reduced pressure to give a residue. The residue was purified by column chromatography (SiO₂, Petroleum ether/Ethyl acetate=1/0 to 10/1) to give compound tert-butyl 2-(3-hydroxyphenoxy)acetate (18 g, 80.27 mmol, 39.14% yield) as a colorless oil.

### Data:

¹H NMR (400 MHz, CHLOROFORM-d) δ=7.18-7.08 (m, 1H), 6.50-6.41 (m, 3H), 4.50 (s, 2H), 1.50 (s, 9H)

### 2. General Procedure for Preparation of tert-butyl 2-[3-[(5-formyl-2-pyridyl)oxy]phenoxy]acetate

To a solution of tert-butyl 2-(3-hydroxyphenoxy)acetate (3.59 g, 15.99 mmol, 1 eq), 6-fluoropyridine-3-carbaldehyde (2 g, 15.99 mmol, 1 eq) in ACN (40 mL) was added K₂CO₃ (6.63 g, 47.96 mmol, 3 eq). The mixture was stirred at 15° C. for 12 hr. The mixture was diluted with water 10 mL, and then extracted with EtOAc 100 mL (20 mL*5). The combined organic layers were washed with sat·NaCl 10 mL, dried over Na₂SO₄, filtered and concentrated under reduced pressure to give a residue. The residue was purified by flash silica gel chromatography (ISCO; 40 g SepaFlash Silica Flash Column, Eluent of 0˜16% Ethyl acetate/Petroleum ether gradient @ 100 mL/min) to give compound tert-butyl 2-[3-[(5-formyl-2-pyridyl)oxy]phenoxy]acetate (4.9 g, 14.88 mmol, 93.06% yield) as a colorless oil.

### 3. General Procedure for Preparation of methyl (2S)-2-[[6-[3-(2-tert-butoxy-2-oxo-ethoxy)phenoxy]-3-pyridyl]methylamino]-5,5-dimethyl-hexanoate

TEA (122.90 mg, 1.21 mmol, 169.05 μL, 1 eq) was added to a solution of methyl (2S)-2-amino-5,5-dimethyl-hexanoate (210.42 mg, 1.21 mmol, 1 eq) in MeOH (4 mL) until pH=8˜9, then the reaction was stirred for 10 min at 15° C., then tert-butyl 2-[3-[(5-formyl-2-pyridyl)oxy]phenoxy]acetate (400 mg, 1.21 mmol, 1 eq), AcOH (72.93 mg, 1.21 mmol, 69.53 μL, 1 eq) was added to adjust pH <5. Then the reaction was stirred for 10 min at 15° C., and NaBH3CN (76.32 mg, 1.21 mmol, 1 eq) were added to the reaction mixture, and the reaction was stirred for 1 hr 40 min at 15° C. The mixture was diluted with water 10 mL, and then extracted with EtOAc 30 mL (10 mL*3). The combined organic layers were washed with sat·NaCl 10 mL, dried over Na₂SO₄, filtered and concentrated under reduced pressure to give a residue. The residue was purified by prep-TLC (SiO₂, Petroleum ether: Ethyl acetate=1:1) to give compound methyl (2S)-2-[[6-[3-(2-tert-butoxy-2-oxo-ethoxy)phenoxy]-3-pyridyl]methylamino]-5,5-dimethyl-hexanoate (375 mg, 760.67 μmol, 62.63% yield, 98.704% purity) as a colorless oil.

### Data:

¹H NMR (400 MHz, CHLOROFORM-d) δ=8.14 (d, J=2.0 Hz, 1H), 7.70 (dd, J=2.4, 8.4 Hz, 1H), 7.34-7.29 (m, 1H), 6.87 (d, J=8.3 Hz, 1H), 6.79-6.72 (m, 2H), 6.71-6.68 (m, 1H), 4.50 (s, 2H), 3.81-3.74 (m, 4H), 3.58 (d, J=13.2 Hz, 1H), 3.21 (t, J=6.6 Hz, 1H), 1.70-1.53 (m, 2H), 1.49 (s, 9H), 1.30 (s, 1H), 1.15 (dt, J=5.2, 12.7 Hz, 1H), 0.87 (s, 9H)

### 4. General Procedure for Preparation of 2-[3-[[5-[[[(1S)-1-methoxycarbonyl-4,4-dimethyl-pentyl]amino]methyl]-2-pyridyl]oxy]phenoxy]acetic acid

A stirred solution of methyl (2S)-2-[[6-[3-(2-tert-butoxy-2-oxo-ethoxy)phenoxy]-3-pyridyl]methylamino]-5,5-dimethyl-hexanoate (365 mg, 750.10 μmol, 1 eq) in DCM (2 mL) and TFA (1 mL) was stirred for 1 hr at 25° C. The DCM and TFA was removed under reduced pressure to give compound 2-[3-[[5-[[[(1S)-1-methoxycarbonyl-4,4-dimethyl-pentyl]amino]methyl]-2-pyridyl]oxy]phenoxy]acetic acid (408 mg, crude, TFA) as a colorless oil.

### 5. General Procedure for Preparation of methyl (2S)-5,5-dimethyl-2-[[6-[3-[2-oxo-2-[2-[2-[2-[2-[2-(2-prop-2-ynoxyethoxy)ethoxy]ethoxy]ethoxy]ethoxy]ethylamino]ethoxy]phenoxy]-3-pyridyl]methylamino]hexanoate

To a solution of 2-[3-[[5-[[[(1S)-1-methoxycarbonyl-4,4-dimethyl-pentyl]amino]methyl]-2-pyridyl]oxy]phenoxy]acetic acid (390 mg, 716.23 μmol, 1 eq, TFA),2-[2-[2-[2-[2-(2-prop-2-ynoxyethoxy)ethoxy]ethoxy]ethoxy]ethoxylethanamine (228.76 mg, 716.23 μmol, 1 eq) in DMF (4 mL) was added DIEA (277.70 mg, 2.15 mmol, 374.26 μL, 3 eq) and CMPI (274.48 mg, 1.07 mmol, 1.5 eq) at 0° C. The mixture was stirred at 25° C. for 4 hr. The mixture was filtered and the filtrate was purified by reversed-phase HPLC (column: C18 20-35 um 100 A 120 g; mobile phase: [water-ACN]; B %: 0%-60% @ 120 mL/min) to give compound methyl (2S)-5,5-dimethyl-2-[[6-[3-[2-oxo-2-[2-[2-[2-[2-[2-(2-prop-2-ynoxyethoxy)ethoxy]ethoxy]ethoxy]ethoxy]ethylamino]ethoxy]phenoxy]-3-pyridyl]methylamino]hexanoate (290 mg, 396.25 μmol, 55.32% yield) as a colorless oil.

### Data:

LCMS (ESI+): m/z 732.6 (M+H)

### 6. General Procedure for Preparation of methyl (2S)-2-[[6-[3-[2-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethoxy]ethoxy]ethylamino]-2-oxo-ethoxy]phenoxy]-3-pyridyl]methylamino]-5,5-dimethyl-hexanoate

To a solution of methyl (2S)-5,5-dimethyl-2-[[6-[3-[2-oxo-2-[2-[2-[2-[2-[2-(2-prop-2-ynoxyethoxy)ethoxy]ethoxy]ethoxy]ethoxy]ethylamino]ethoxy]phenoxy]-3-pyridyl]methylamino]hexanoate (80 mg, 109.31 μmol, 1 eq),3-[(1R)-1-[[6-[2-(5-azidopentoxy)pyrimidin-5-yl]-3-chloro-7-fluoro-2-methyl-1,5-naphthyridin-4-yl]amino]ethyl]-4-fluoro-benzonitrile (61.65 mg, 109.31 μmol, 1 eq) in t-BuOH (0.5 mL) and H₂O (0.5 mL) was added copper; sulfate (8.72 mg, 54.65 μmol, 8.39 μL, 0.5 eq) and sodium; (2R)-2-[(1S)-1,2-dihydroxyethyl]-4-hydroxy-5-oxo-2H-furan-3-olate (21.65 mg, 109.31 μmol, 1 eq). The mixture was stirred at 50° C. for 0.5 hr. The mixture was filtered and the filtrate was purified by prep-HPLC. The product was purified by reversed-phase HPLC (column: C18 20-35 um 100 A 40 g; mobile phase: [water-ACN]; B %: 0%-99% @ 65 mL/min) to give compound methyl (2S)-2-[[6-[3-[2-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethoxy]ethoxy]ethylamino]-2-oxo-ethoxy]phenoxy]-3-pyridyl]methylamino]-5,5-dimethyl-hexanoate (75 mg, 57.88 μmol, 52.95% yield) as a colorless oil.

### Data:

LCMS (ESI+): m/z 1295.5 (M+H)

### 7. General Procedure for Preparation of (2S)-2-[[6-[3-[2-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethoxy]ethoxy]ethylamino]-2-oxo-ethoxy]phenoxy]-3-pyridyl]methylamino]-5,5-dimethyl-hexanoic acid

To a solution of methyl (2S)-2-[[6-[3-[2-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethoxy]ethoxy]ethylamino]-2-oxo-ethoxy]phenoxy]-3-pyridyl]methylamino]-5,5-dimethyl-hexanoate (70 mg, 54.02 μmol, 1 eq) in H₂O (0.3 mL) and THF (0.3 mL) was added LiOH·H₂O (4.53 mg, 108.04 μmol, 2 eq). The mixture was stirred at 15° C. for 4 hr. The residue was acidified with HCl (1M) to pH=5. The mixture was filtered and the filtrate was purified by prep-HPLC. The residue was purified by prep-HPLC (neutral condition; column: Waters Xbridge BEH C18 100*30 mm*10 um; mobile phase: [H₂O (10 mM NH₄HCO₃)-ACN]; gradient: 30%-75% B over 8.0 min) to give compound (2S)-2-[[6-[3-[2-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethoxy]ethoxy]ethylamino]-2-oxo-ethoxy]phenoxy]-3-pyridyl]methylamino]-5,5-dimethyl-hexanoic acid (35.9 mg, 27.86 μmol, 51.57% yield, 99.472% purity) as a pale yellow solid.

### Data:

¹H NMR (400 MHz, METHANOL-d4) δ=8.99 (d, J=1.2 Hz, 2H), 8.24 (d, J=2.3 Hz, 1H), 8.01 (s, 1H), 7.98-7.88 (m, 2H), 7.78 (dd, J=2.1, 6.9 Hz, 1H), 7.66-7.57 (m, 1H), 7.32 (t, J=8.2 Hz, 1H), 7.18 (dd, J=8.6, 10.2 Hz, 1H), 7.00 (d, J=8.6 Hz, 1H), 6.86 (dd, J=2.1, 8.1 Hz, 1H), 6.80-6.70 (m, 2H), 6.44 (d, J=6.8 Hz, 1H), 4.62 (s, 2H), 4.54-4.44 (m, 6H), 4.26-4.10 (m, 2H), 3.67-3.52 (m, 23H), 3.47-3.42 (m, 2H), 2.70 (s, 3H), 2.03 (quin, J=7.3 Hz, 2H), 1.94-1.78 (m, 4H), 1.72 (d, J=6.8 Hz, 3H), 1.57-1.48 (m, 2H), 1.46-1.24 (m, 2H), 0.90 (s, 9H)

LCMS (ESI+): m/z 1281.5 (M+H)

### BF149

### 1. General Procedure for Preparation of tert-butyl 2-[2-[2-[2-[2-[2-(2-prop-2-ynoxyethoxy)ethoxy]ethoxy]ethoxy]ethoxy]ethoxy]acetate

To a solution of tert-butyl 2-[2-[2-[2-[2-[2-(2-hydroxyethoxy)ethoxy]ethoxy]ethoxy]ethoxy]ethoxy]acetate (2 g, 5.04 mmol, 1 eq) and 3-bromoprop-1-yne (600.09 mg, 5.04 mmol, 434.85 μL, 1 eq) in DMF (25 mL) was added NaH (201.76 mg, 5.04 mmol, 60% purity, 1 eq) at 0° C. The mixture was stirred at 0° C. for 1 hr. The reaction mixture was added to sat·NH₄Cl (40 mL), extracted with ethyl acetate 90 mL (30 mL*3), washed with brine 150 mL (50 mL*3). The organic layer was dried over Na₂SO₄, concentrated to give a residue. The residue was purified by column chromatography (SiO₂, Petroleum ether/Ethyl acetate=5/1 to 0/1) to give compound tert-butyl 2-[2-[2-[2-[2-[2-(2-prop-2-ynoxyethoxy)ethoxy]ethoxy]ethoxy]ethoxy]ethoxy]acetate (1 g, 2.30 mmol, 45.62% yield) as a yellow liquid.

### Data:

¹H NMR (400 MHz, CHLOROFORM-d) δ=4.20 (d, J=2.4 Hz, 2H), 4.01 (s, 2H), 3.70-3.66 (m, 8H), 3.64 (d, J=3.3 Hz, 16H), 2.43 (t, J=2.3 Hz, 1H), 1.46 (s, 9H)

### 2. General Procedure for Preparation of 2-[2-[2-[2-[2-[2-(2-prop-2-ynoxyethoxy)ethoxy]ethoxy]ethoxy]ethoxy]ethoxy]acetic acid

A mixture of tert-butyl 2-[2-[2-[2-[2-[2-(2-prop-2-ynoxyethoxy)ethoxy]ethoxy]ethoxy]ethoxy]ethoxy]acetate (1 g, 2.30 mmol, 1 eq) in TFA (2 mL) and DCM (10 mL) was stirred at 25° C. for 1 hr. The reaction mixture was concentrated to give compound 2-[2-[2-[2-[2-[2-(2-prop-2-ynoxyethoxy)ethoxy]ethoxy]ethoxy]ethoxy]ethoxy]acetic acid (900 mg, crude) as a brown oil.

### 3. General Procedure for Preparation of tert-butyl(2S)-3-phenyl-2-[[2-[2-[2-[2-[2-[2-(2-prop-2-ynoxyethoxy)ethoxy]ethoxy]ethoxy]ethoxy]ethoxy]acetyl]amino]propanoate

To a solution of 2-[2-[2-[2-[2-[2-(2-prop-2-ynoxyethoxy)ethoxy]ethoxy]ethoxy]ethoxy]ethoxy]acetic acid (900 mg, 2.38 mmol, 1 eq) and tert-butyl(2S)-2-amino-3-phenyl-propanoate (526.32 mg, 2.38 mmol, 1 eq) in DMF (10 mL) was added HATU (2.71 g, 7.14 mmol, 3 eq) and DIEA (922.16 mg, 7.14 mmol, 1.24 mL, 3 eq) at 0° C. The mixture was stirred at 25° C. for 1 hr. The reaction mixture was filtered and the filtrate was purified by reversed-phase HPLC (column: C18 20-35 um 100 A 40 g; mobile phase: [water-ACN]; B %: 0%-75% @ 80 mL/min) to give compound (2S)-3-phenyl-2-[[2-[2-[2-[2-[2-[2-(2-prop-2-tert-butyl ynoxyethoxy)ethoxy]ethoxy]ethoxy]ethoxy]ethoxy]acetyl]amino]propanoate (300 mg, 429.39 μmol, 18.05% yield, 83.258% purity) as a brown oil.

### Data:

LCMS (ESI⁺): m/z 582.3 (M+H)

### 4. General Procedure for Preparation of (2S)-3-phenyl-2-[[2-[2-[2-[2-[2-[2-(2-prop-2-ynoxyethoxy)ethoxy]ethoxy]ethoxy]ethoxy]ethoxy]acetyl]amino]propanoic acid

A mixture of tert-butyl(2S)-3-phenyl-2-[[2-[2-[2-[2-[2-[2-(2-prop-2-ynoxyethoxy)ethoxy]ethoxy]ethoxy]ethoxy]ethoxy]acetyl]amino]propanoate (280 mg, 400.77 μmol, 1 eq) in TFA (0.5 mL) and DCM (2.5 mL) was stirred at 25° C. for 2 hr. The reaction mixture was concentrated to give compound (2S)-3-phenyl-2-[[2-[2-[2-[2-[2-[2-(2-prop-2-ynoxyethoxy)ethoxy]ethoxy]ethoxy]ethoxy]ethoxy]acetyl]amino]propanoic acid (250 mg, crude) was obtained as brown oil.

### Data:

LCMS (ESI⁺): m/z 526.2 (M+H)

### 5. General Procedure for Preparation of methyl (2S)-5,5-dimethyl-2-[[(2S)-3-phenyl-2-[[2-[2-[2-[2-[2-[2-(2-prop-2-ynoxyethoxy)ethoxy]ethoxy]ethoxy]ethoxy]ethoxy]acetyl]amino]propanoyl]amino]hexanoate

To a solution of (2S)-3-phenyl-2-[[2-[2-[2-[2-[2-[2-(2-prop-2-ynoxyethoxy)ethoxy]ethoxy]ethoxy]ethoxy]ethoxy]acetyl]amino]propanoic acid (225 mg, 428.09 μmol, 1 eq) and methyl (2S)-2-amino-5,5-dimethyl-hexanoate (74.17 mg, 428.09 μmol, 1 eq) in DMF (3 mL) was added HATU (488.32 mg, 1.28 mmol, 3 eq) and DIEA (165.98 mg, 1.28 mmol, 223.70 μL, 3 eq) at 0° C. The mixture was stirred at 25° C. for 1 hr. LCMS showed Reactant 1 was consumed completely and one main peak with desired mass was detected. The reaction mixture was filtered and the filtrate was purified by prep-HPLC (basic condition: column: Phenomenex Gemini NX-C18 (75*30 mm*3 um); mobile phase: [H₂O (0.05% NH₃H₂O+10 mM NH₄HCO₃)-ACN]; gradient: 35%-65% B over 8.0 min) to give compound methyl (2S)-5,5-dimethyl-2-[[(2S)-3-phenyl-2-[[2-[2-[2-[2-[2-[2-(2-prop-2-ynoxyethoxy)ethoxy]ethoxy]ethoxy]ethoxy]ethoxy]acetyl]amino]propanoyl]amino]hexan oate (126 mg, 185.07 μmol, 43.23% yield) as a brown oil

### Data:

LCMS (ESI⁺): m/z 681.3 (M+H)

¹H NMR (400 MHz, METHANOL-d4) δ=7.28 (d, J=4.4 Hz, 4H), 7.22 (qd, J=4.2, 8.5 Hz, 1H), 4.76 (dd, J=5.4, 9.0 Hz, 1H), 4.35 (dd, J=5.3, 8.4 Hz, 1H), 4.18 (d, J=2.4 Hz, 2H), 4.01-3.82 (m, 2H), 3.71 (s, 3H), 3.69-3.57 (m, 24H), 3.19 (dd, J=5.4, 13.9 Hz, 1H), 2.97 (dd, J=9.0, 13.9 Hz, 1H), 2.84 (t, J=2.4 Hz, 1H), 1.89-1.76 (m, 1H), 1.74-1.63 (m, 1H), 1.33-1.16 (m, 2H), 0.90 (s, 9H)

### 6. General Procedure for Preparation of methyl (2S)-2-[[(2S)-2-[[2-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]meth-oxy]ethoxy]ethoxy]ethoxy]ethoxy]ethoxy]ethoxy]acetyl]amino]-3-phenyl-propanoyl]amino]-5,5-dimethyl-hexanoate

To a solution of methyl (2S)-5,5-dimethyl-2-[[(2S)-3-phenyl-2-[[2-[2-[2-[2-[2-[2-(2-prop-2-ynoxyethoxy)ethoxy]ethoxy]ethoxy]ethoxy]ethoxy]acetyl]amino]propanoyl]amino]hexan oate (55 mg, 80.78 μmol, 1 eq) and 3-[(1R)-1-[[6-[2-(5-azidopentoxy)pyrimidin-5-yl]-3-chloro-7-fluoro-2-methyl-1,5-naphthyridin-4-yl]amino]ethyl]-4-fluoro-benzonitrile (45.56 mg, 80.78 μmol, 1 eq) in H₂O (0.2 mL) and t-BuOH (0.2 mL) was added CuSO₄·5H₂O (20.17 mg, 80.78 μmol, 1 eq) and sodium; (2R)-2-[(1S)-1,2-dihydroxyethyl]-4-hydroxy-5-oxo-2H-furan-3-olate (16.00 mg, 80.78 μmol, 1 eq). The mixture was stirred at 50° C. for 1 hr. The reaction mixture was filtered and the filtrate was purified by reversed-phase HPLC (column: C18 20-35 um 100 A 40 g; mobile phase: [water-ACN]; B %: 0%-75% @ 80 mL/min) to give compound methyl (2S)-2-[[(2S)-2-[[2-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethoxy]ethoxy]ethoxy]acetyl]amino]-3-phenyl-propanoyl]amino]-5,5-dimethyl-hexanoate (80 mg, 64.27 μmol, 79.55% yield) as a colorless oil.

### Data:

LCMS (ESI⁺): m/z 1244.5 (M+H)

### 7. General Procedure for Preparation of (2S)-2-[[(2S)-2-[[2-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]meth-oxy]ethoxy]ethoxy]ethoxy]ethoxy]ethoxy]ethoxy]acetyl]amino]-3-phenyl-propanoyl]amino]-5,5-dimethyl-hexanoic acid

To a solution of methyl (2S)-2-[[(2S)-2-[[2-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethoxy]ethoxy]ethoxy]acetyl]amino]-3-phenyl-propanoyl]amino]-5,5-dimethyl-hexanoate (115 mg, 92.38 μmol, 1 eq) in THF (1 mL) and H₂O (1 mL) was added LiOH·H₂O (15.51 mg, 369.53 μmol, 4 eq) at 0° C. The mixture was stirred at 0° C. for 1 hr. The mixture was acidified with FA until pH=3. The reaction mixture was filtered and the filtrate was purified by prep-HPLC (FA condition: column: column: Phenomenex Luna C18 100*30 mm*3 um; mobile phase: [H₂O (0.2% FA)-ACN]; gradient: 55%-90% B over 8.0 min) to give compound (2S)-2-[[(2S)-2-[[2-[2-[2-[2-[2-[2-[2-[[1-[5-[5-[7-chloro-8-[[(1R)-1-(5-cyano-2-fluoro-phenyl)ethyl]amino]-3-fluoro-6-methyl-1,5-naphthyridin-2-yl]pyrimidin-2-yl]oxypentyl]triazol-4-yl]methoxy]ethoxy]ethoxy]ethoxy]ethoxy]ethoxy]ethoxy]acetyl]amino]-3-phenyl-propanoyl]amino]-5,5-dimethyl-hexanoic acid (23.5 mg, 18.90 μmol, 20.46% yield, 99.005% purity) as a yellow gum.

### Data:

LCMS (ESI⁺): m/z 1230.5 (M+H)

¹H NMR (400 MHz, METHANOL-d4) δ=8.97 (d, J=0.8 Hz, 2H), 8.01 (s, 1H), 7.87 (d, J=11.6 Hz, 1H), 7.78 (dd, J=1.9, 6.9 Hz, 1H), 7.61 (ddd, J=2.1, 4.8, 8.5 Hz, 1H), 7.29-7.13 (m, 6H), 6.41 (q, J=6.8 Hz, 1H), 4.76 (dd, J=5.0, 9.3 Hz, 1H), 4.63 (s, 2H), 4.48 (td, J=6.7, 9.6 Hz, 4H), 4.33 (dd, J=5.1, 8.1 Hz, 1H), 4.01-3.77 (m, 2H), 3.66-3.55 (m, 23H), 3.52-3.46 (m, 1H), 3.20 (dd, J=5.0, 13.9 Hz, 1H), 2.96 (dd, J=9.3, 14.0 Hz, 1H), 2.68 (s, 3H), 2.03 (quin, J=7.4 Hz, 2H), 1.96-1.79 (m, 3H), 1.76-1.63 (m, 4H), 1.59-1.48 (m, 2H), 1.30-1.20 (m, 2H), 0.88 (s, 9H)

### Example S2: Characterization of Compounds

The following compounds were prepared according similar procedures as described herein above. A person of skill in the art will readily be aware of the multiple reactions that enable connection of different groups and find the guidance in the above-described methods to produce compounds according to the present disclosure. The starting materials are either commercially available or may be prepared from commercially available reagents using conventional reactions well known in the art.

As proof of disclosure, below are included the NMR and LC/MS characterization of the compounds prepared:

| | | LC/MS |
|---|---|---|
| ID | ¹H NMR | [M + 1] |
| BF001 | 1H NMR (400 MHz, METHANOL-d4) δ = 8.97 (d, J = 2.6 Hz, | 697.3 |
| | 1H), 8.59 (d, J = 2.0 Hz, 1H), 8.24 (ddd, J = 2.6, 6.3, 9.0 Hz, | |
| | 2H), 7.69 (td, J = 1.3, 7.8 Hz, 1H), 7.59 (t, J = 1.9 Hz, 1H), 7.48 | |
| | (t, J = 7.9 Hz, 1H), 7.28 (ddd, J = 0.9, 2.4, 8.1 Hz, 1H), 7.12- | |
| | 7.03 (m, 2H), 4.50 (dd, J = 5.2, 8.8 Hz, 1H), 3.80 (t, J = 5.3 Hz, | |
| | 2H), 3.72-3.64 (m, 6H), 3.57 (q, J = 5.8 Hz, 4H), 2.05-1.89 (m, | |
| | 1H), 1.88-1.74 (m, 1H), 1.44-1.27 (m, 2H), 0.92 (s, 9H) | |
| BF002 | 1H NMR (400 MHz, METHANOL-d4) δ = 8.99 (d, J = 2.6 Hz, | 741.3 |
| | 1H), 8.60 (d, J = 2.5 Hz, 1H), 8.25 (ddd, J = 2.6, 6.5, 9.1 Hz, | |
| | 2H), 7.70 (dd, J = 1.1, 7.7 Hz, 1H), 7.60 (t, J = 1.9 Hz, 1H), 7.50 | |
| | (t, J = 7.9 Hz, 1H), 7.32-7.28 (m, 1H), 7.15 (d, J = 9.6 Hz, 1H), | |
| | 7.06 (d, J = 8.8 Hz, 1H), 4.51 (dd, J = 5.2, 8.8 Hz, 1H), 3.78- | |
| | 3.70 (m, 2H), 3.69-3.51 (m, 14H), 2.09-1.87 (m, 1H), 1.80 (s, | |
| | 1H), 1.35 (ddd, J = 5.1, 9.1, 11.8 Hz, 2H), 0.92 (s, 9H) | |
| BF003 | 1H NMR (400 MHz, METHANOL-d4) δ = 9.01 (d, J = 2.6 Hz, | 785.3 |
| | 1H), 8.61 (d, J = 2.4 Hz, 1H), 8.29-8.24 (m, 2H), 7.72 (d, J = | |
| | 7.9 Hz, 1H), 7.62 (s, 1H), 7.52 (s, 1H), 7.33 (br d, J = 2.0 Hz, | |
| | 1H), 7.18 (d, J = 9.6 Hz, 1H), 7.07 (d, J = 8.6 Hz, 1H), 4.49 (dd, | |
| | J = 5.2, 8.3 Hz, 1H), 3.79-3.73 (m, 2H), 3.66-3.54 (m, 18H), | |
| | 2.01-1.90 (m, 1H), 1.86-1.72 (m, 1H), 1.36-1.29 (m, 2H), | |
| | 0.91 (s, 9H) | |
| BF004 | 1H NMR (400 MHz, METHANOL-d4) δ = 9.01 (d, J = 2.5 Hz, | 802.4 |
| | 1H), 8.64 (d, J = 2.4 Hz, 1H), 8.27 (td, J = 2.8, 9.0 Hz, 2H), 7.32 | |
| | (t, J = 8.3 Hz, 1H), 7.19 (d, J = 9.5 Hz, 1H), 7.00 (d, J = 8.6 Hz, | |
| | 1H), 6.88-6.82 (m, 1H), 6.78-6.69 (m, 2H), 4.51 (dd, J = 5.1, | |
| | 8.7 Hz, 1H), 4.14-4.08 (m, 2H), 3.80 (td, J = 4.9, 17.2 Hz, 4H), | |
| | 3.69-3.58 (m, 18H), 2.02-1.90 (m, 1H), 1.87-1.76 (m, 1H), | |
| | 1.40-1.29 (m, 2H), 0.92 (s, 9H). | |
| BF005 | 1H NMR (400 MHz, METHANOL-d4) δ = 9.00 (d, J = 2.8 Hz, | 876.2 |
| | 1H), 8.55 (d, J = 2.0 Hz, 1H), 8.28 (dd, J = 2.6, 8.7 Hz, 1H), 8.23 | |
| | (dd, J = 2.8, 9.6 Hz, 1H), 8.13 (d, J = 8.6 Hz, 1H), 7.98 (d, J = | |
| | 8.5 Hz, 1H), 7.67 (dd, J = 1.1, 7.1 Hz, 1H), 7.63-7.56 (m, 1H), | |
| | 7.50 (dd, J = 7.1, 8.5 Hz, 1H), 7.32 (d, J = 7.0 Hz, 1H), 7.13 (dd, | |
| | J = 2.8, 9.1 Hz, 2H), 4.50 (dd, J = 5.2, 8.8 Hz, 1H), 3.77-3.71 | |
| | (m, 4H), 3.67-3.58 (m, 10H), 3.52 (t, J = 6.8 Hz, 2H), 3.34 (br d, | |
| | J = 6.9 Hz, 2H), 2.46 (t, J = 6.0 Hz, 2H), 2.00-1.78 (m, 4H), | |
| | 1.40-1.26 (m, 2H), 0.91 (s, 9H) | |
| BF006 | 1H NMR (400 MHz, METHANOL-d4) δ = 9.01 (br d, J = 2.6 Hz, | 917.5 |
| | 1H), 8.70-8.58 (m, 1H), 8.36-8.17 (m, 2H), 7.74 (br d, J = 7.8 | |
| | Hz, 1H), 7.64 (br d, J = 1.8 Hz, 1H), 7.54 (br t, J = 8.0 Hz, 1H), | |
| | 7.37-7.30 (m, 1H), 7.20 (br d, J = 9.6 Hz, 1H), 7.09 (br d, J = | |
| | 8.6 Hz, 1H), 4.51 (br dd, J = 5.1, 8.6 Hz, 1H), 3.81-3.77 (m, | |
| | 3H), 3.71-3.53 (m, 29H), 2.02-1.93 (m, 1H), 1.87-1.75 (m, | |
| | 1H), 1.41-1.30 (m, 2H), 0.92 (s, 9H). | |
| BF007 | 1H NMR (400 MHz, METHANOL-d4) δ = 8.97 (d, J = 2.7 Hz, | 714.3 |
| | 1H), 8.62 (d, J = 2.3 Hz, 1H), 8.28-8.19 (m, 2H), 7.29 (t, J = 8.1 | |
| | Hz, 1H), 7.16 (d, J = 9.5 Hz, 1H), 6.98 (d, J = 8.8 Hz, 1H), 6.81 | |
| | (dd, J = 2.1, 8.3 Hz, 1H), 6.74-6.65 (m, 2H), 4.51 (dd, J = 5.1, | |
| | 8.8 Hz, 1H), 4.14-4.05 (m, 2H), 3.80 (td, J = 4.9, 13.4 Hz, 4H), | |
| | 3.71-3.58 (m, 10H), 2.01-1.89 (m, 1H), 1.87-1.74 (m, 1H), | |
| | 1.43-1.26 (m, 2H), 0.92 (s, 9H) | |
| BF008 | 1H NMR (400 MHz, METHANOL-d4) δ = 9.02 (d, J = 2.6 Hz, | 873.2 |
| | 1H), 8.63 (d, J = 2.3 Hz, 1H), 8.28 (dt, J = 2.5, 8.8 Hz, 2H), 7.74 | |
| | (d, J = 7.9 Hz, 1H), 7.64 (t, J = 1.9 Hz, 1H), 7.54 (t, J = 7.9 Hz, | |
| | 1H), 7.34 (dd, J = 1.6, 8.1 Hz, 1H), 7.20 (d, J = 9.6 Hz, 1H), 7.09 | |
| | (d, J = 8.8 Hz, 1H), 4.51 (dd, J = 5.2, 8.7 Hz, 1H), 3.82-3.77 (m, | |
| | 2H), 3.69-3.54 (m, 26H), 2.05-1.91 (m, 1H), 1.87-1.76 (m, | |
| | 1H), 1.41-1.28 (m, 2H), 0.92 (s, 9H) | |
| BF009 | 1H NMR (400 MHz, METHANOL-d4) δ ppm 9.00 (t, J = 2.25 Hz, | 832.3 |
| | 1 H), 8.57 (d, J = 2.38 Hz, 1 H), 8.30 (dd, J = 8.63, 2.50 Hz, 1 H), | |
| | 8.18-8.25 (m, 1 H), 8.14 (d, J = 8.63 Hz, 1 H), 7.99 (d, J = 8.50 | |
| | Hz, 1 H), 7.68 (dd, J = 7.07, 1.06 Hz, 1 H), 7.57-7.64 (m, 1 H), | |
| | 7.51 (dd, J = 8.50, 7.13 Hz, 1 H), 7.29-7.36 (m, 1 H), 7.08-7.17 | |
| | (m, 2 H), 4.52 (dd, J = 8.88, 5.13 Hz, 1 H), 3.75-3.82 (m, 4 H), | |
| | 3.64-3.73 (m, 4 H), 3.62 (t, J = 5.25 Hz, 2 H), 3.53 (t, J = 6.69 Hz, | |
| | 2 H), 3.35-3.40 (m, 2 H), 2.49 (t, J = 6.07 Hz, 2 H), 1.77-2.02 | |
| | (m, 4 H), 1.27-1.45 (m, 2 H), 0.93 (s, 9 H). | |
| BF010 | 1H NMR (400 MHz, METHANOL-d4) δ = 8.99 (d, J = 2.8 Hz, | 920.4 |
| | 1H), 8.63-8.57 (m, 1H), 8.36-8.30 (m, 1H), 8.23 (dd, J = 2.7, | |
| | 9.6 Hz, 1H), 8.16 (d, J = 8.5 Hz, 1H), 8.00 (d, J = 8.5 Hz, 1H), | |
| | 7.69 (dd, J = 1.0, 7.0 Hz, 1H), 7.65-7.59 (m, 1H), 7.52 (dd, J = | |
| | 7.1, 8.4 Hz, 1H), 7.36 (dd, J = 0.8, 7.5 Hz, 1H), 7.18-7.11 (m, | |
| | 2H), 4.56-4.47 (m, 1H), 3.78-3.71 (m, 4H), 3.66-3.58 (m, | |
| | 14H), 3.53 (t, J = 6.8 Hz, 2H), 3.37 (t, J = 6.8 Hz, 2H), 2.48 (t, J = | |
| | 6.1 Hz, 2H), 2.01-1.77 (m, 4H), 1.39-1.29 (m, 2H), 0.94- | |
| | 0.90 (m, 9H). | |
| BF011 | 1H NMR (400 MHz, METHANOL-d4) δ = 8.99 (d, J = 2.6 Hz, | 829.2 |
| | 1H), 8.61 (d, J = 2.4 Hz, 1H), 8.26 (ddd, J = 2.6, 9.1, 11.5 Hz, | |
| | 2H), 7.73 (d, J = 7.8 Hz, 1H), 7.63 (t, J = 1.9 Hz, 1H), 7.52 (t, J = | |
| | 7.9 Hz, 1H), 7.33 (dd, J = 1.7, 8.1 Hz, 1H), 7.17 (d, J = 9.6 Hz, | |
| | 1H), 7.07 (d, J = 8.6 Hz, 1H), 4.51 (dd, J = 5.3, 8.8 Hz, 1H), 3.79- | |
| | 3.74 (m, 2H), 3.68-3.61 (m, 12H), 3.60-3.53 (m, 10H), 2.01- | |
| | 1.90 (m, 1H), 1.87-1.75 (m, 1H), 1.42-1.27 (m, 2H), 0.92 (s, | |
| | 9H) | |
| BF012 | 1H NMR (400 MHz, METHANOL-d4) δ = 9.00 (d, J = 2.6 Hz, | 890.4 |
| | 1H), 8.63 (d, J = 2.5 Hz, 1H), 8.26 (ddd, J = 2.6, 6.8, 9.2 Hz, | |
| | 2H), 7.32 (t, J = 8.2 Hz, 1H), 7.20 (d, J = 9.6 Hz, 1H), 6.99 (d, J = | |
| | 8.6 Hz, 1H), 6.85 (dd, J = 2.4, 8.3 Hz, 1H), 6.77-6.69 (m, 2H), | |
| | 4.51 (dd, J = 5.3, 8.8 Hz, 1H), 4.16-4.10 (m, 2H), 3.85-3.82 | |
| | (m, 2H), 3.81-3.77 (m, 2H), 3.71-3.62 (m, 12H), 3.61-3.57 | |
| | (m, 14H), 2.01-1.91 (m, 1H), 1.87-1.76 (m, 1H), 1.35 (ddd, J = | |
| | 5.0, 9.4, 11.7 Hz, 2H), 0.92 (s, 9H) | |
| BF013 | 1H NMR (400 MHz, METHANOL-d4) δ = 8.97 (d, J = 2.8 Hz, | 670.3 |
| | 1H), 8.62 (d, J = 2.0 Hz, 1H), 8.24 (dd, J = 2.6, 9.6 Hz, 2H), 7.29 | |
| | (t, J = 8.1 Hz, 1H), 7.17 (d, J = 9.6 Hz, 1H), 6.98 (d, J = 8.8 Hz, | |
| | 1H), 6.81 (dd, J = 1.8, 8.3 Hz, 1H), 6.73-6.66 (m, 2H), 4.51 (dd, | |
| | J = 5.2, 8.8 Hz, 1H), 4.11 (dd, J = 3.8, 5.4 Hz, 2H), 3.86-3.79 | |
| | (m, 4H), 3.75-3.69 (m, 4H), 3.66-3.61 (m, 2H), 1.96 (s, 1H), | |
| | 1.88-1.76 (m, 1H), 1.36 (ddd, J = 4.9, 9.6, 11.8 Hz, 2H), 0.92 | |
| | (s, 9H) | |
| BF014 | 1H NMR (400 MHz, METHANOL-d4)δ ppm 8.99-9.04 (m, 1 H), | 846.3 |
| | 8.65-8.72 (m, 1 H), 8.24-8.34 (m, 2 H), 7.35 (t, J = 8.19 Hz, 1 | |
| | H), 7.21 (d, J = 9.63 Hz, 1 H), 7.04 (d, J = 8.63 Hz, 1 H), 6.88 (dd, | |
| | J = 8.38, 1.75 Hz, 1 H), 6.73-6.82 (m, 2 H), 4.54 (ddd, J = 8.60, | |
| | 5.41, 3.00 Hz, 1 H), 4.10-4.19 (m, 2 H), 3.83-3.86 (m, 2 H), | |
| | 3.79-3.82 (m, 2 H), 3.64-3.72 (m, 10 H), 3.59-3.63 (m, 12 H), | |
| | 1.89-2.03 (m, 1 H), 1.77-1.89 (m, 1 H), 1.26-1.44 (m, 2 H), | |
| | 0.93 (s, 9 H). | |
| BF015 | 1H NMR (400 MHz, METHANOL-d4) δ = 8.98-8.94 (m, 1H), | 788.3 |
| | 8.55 (d, J = 2.4 Hz, 1H), 8.30-8.27 (m, 1H), 8.22-8.17 (m, 1H), | |
| | 8.11 (d, J = 8.6 Hz, 1H), 7.98 (d, J = 8.5 Hz, 1H), 7.65 (d, J = 7.0 | |
| | Hz, 1H), 7.58 (t, J = 8.1 Hz, 1H), 7.49 (dd, J = 7.1, 8.4 Hz, 1H), | |
| | 7.31 (d, J = 7.5 Hz, 1H), 7.15-7.09 (m, 2H), 4.55-4.46 (m, 1H), | |
| | 3.84-3.74 (m, 4H), 3.69-3.62 (m, 2H), 3.49 (t, J = 6.7 Hz, 2H), | |
| | 3.37-3.33 (m, 2H), 2.50 (t, J = 5.9 Hz, 2H), 2.05-1.75 (m, 4H), | |
| | 1.45-1.27 (m, 2H), 0.91 (s, 9H) | |
| BF016 | 1H NMR (400 MHz, METHANOL-d4) δ = 9.02 (d, J = 2.6 Hz, | 744.3 |
| | 1H), 8.56 (d, J = 2.4 Hz, 1H), 8.32-8.25 (m, 2H), 8.12 (d, J = | |
| | 8.6 Hz, 1H), 8.00 (d, J = 8.4 Hz, 1H), 7.67 (dd, J = 1.0, 7.0 Hz, | |
| | 1H), 7.64-7.58 (m, 1H), 7.51 (dd, J = 7.1, 8.4 Hz, 1H), 7.33 (d, | |
| | J = 7.5 Hz, 1H), 7.21 (d, J = 9.6 Hz, 1H), 7.12 (d, J = 8.6 Hz, | |
| | 1H), 4.51 (dd, J = 5.1, 8.8 Hz, 1H), 3.79 (t, J = 6.4 Hz, 2H), 3.48 | |
| | (t, J = 6.8 Hz, 2H), 3.36 (br t, J = 6.8 Hz, 2H), 2.65 (t, J = 6.5 Hz, | |
| | 2H), 2.00-1.78 (m, 4H), 1.34 (ddd, J = 5.1, 8.9, 11.7 Hz, 2H), | |
| | 0.91 (s, 9H) | |
| BF017 | 1H NMR (400 MHz, METHANOL-d4) δ = 8.72 (d, J = 2.3 Hz, | 950.5 |
| | 1H), 8.39 (dd, J = 2.4, 8.8 Hz, 1H), 7.38 (t, J = 8.3 Hz, 1H), 7.07 | |
| | (d, J = 8.8 Hz, 1H), 6.91 (dd, J = 1.9, 8.3 Hz, 1H), 6.85-6.82 (m, | |
| | 1H), 6.79 (dd, J = 2.0, 7.9 Hz, 1H), 4.54 (ddd, J = 4.9, 8.3, 18.4 | |
| | Hz, 2H), 4.37 (dd, J = 4.4, 7.9 Hz, 1H), 4.17-4.12 (m, 2H), 3.86- | |
| | 3.83 (m, 2H), 3.71-3.60 (m, 24H), 3.54 (t, J = 5.4 Hz, 2H), | |
| | 3.40-3.34 (m, 2H), 3.27-3.19 (m, 1H), 2.95 (dd, J = 4.9, 12.9 | |
| | Hz, 1H), 2.74 (d, J = 12.9 Hz, 1H), 2.24 (t, J = 7.3 Hz, 2H), 2.02- | |
| | 1.92 (m, 1H), 1.88-1.78 (m, 1H), 1.74-1.55 (m, 4H), 1.49- | |
| | 1.32 (m, 4H), 0.96-0.89 (m, 9H). | |
| BF018 | 1H NMR (400 MHz, METHANOL-d4) δ = 8.70-8.65 (m, 1H), | 1051.5 |
| | 8.37-8.31 (m, 1H), 7.77 (d, J = 7.8 Hz, 1H), 7.68 (s, 1H), 7.57 | |
| | (t, J = 7.9 Hz, 1H), 7.38 (dd, J = 1.6, 8.1 Hz, 1H), 7.12 (d, J = 8.6 | |
| | Hz, 1H), 4.59 (dd, J = 4.9, 7.9 Hz, 1H), 4.52 (dd, J = 5.1, 8.8 Hz, | |
| | 1H), 4.40 (dd, J = 4.4, 7.9 Hz, 1H), 3.67-3.53 (m, 34H), 3.39- | |
| | 3.35 (m, 2H), 3.28-3.22 (m, 1H), 2.96 (dd, J = 4.9, 12.9 Hz, | |
| | 1H), 2.75 (d, J = 12.9 Hz, 1H), 2.25 (t, J = 7.4 Hz, 2H), 2.01- | |
| | 1.90 (m, 1H), 1.86-1.57 (m, 5H), 1.49-1.30 (m, 4H), 0.92 (s, | |
| | 9H) | |
| BF019 | 1H NMR (400 MHz, METHANOL-d4) δ ppm 8.65 (d, J = 2.38 Hz, | 906.4 |
| | 1 H), 8.26 (dd, J = 8.57, 2.31 Hz, 1 H), 7.34 (t, J = 8.19 Hz, 1 H), | |
| | 7.00 (d, J = 8.75 Hz, 1 H), 6.86 (dd, J = 8.44, 2.31 Hz, 1 H), 6.69- | |
| | 6.80 (m, 2 H), 4.49 (td, J = 8.35, 5.07 Hz, 2 H), 4.29 (dd, J = 7.88, | |
| | 4.50 Hz, 1 H), 4.10-4.17 (m, 2 H), 3.80-3.88 (m, 2 H), 3.68- | |
| | 3.71 (m, 2 H), 3.60-3.67 (m, 16 H), 3.53 (t, J = 5.38 Hz, 2 H), | |
| | 3.33-3.37 (m, 4 H), 3.16-3.23 (m, 1 H), 2.91 (dd, J = 12.82, | |
| | 4.94 Hz, 1 H), 2.69 (d, J = 12.76 Hz, 1 H), 2.21 (t, J = 7.38 Hz, 2 | |
| | H), 1.91-2.02 (m, 1 H), 1.77-1.86 (m, 1 H), 1.54-1.74 (m, 4 | |
| | H), 1.31-1.47 (m, 4 H), 0.92 (s, 9 H). | |
| BF020 | 1H NMR (400 MHz, METHANOL-d4) δ ppm 8.59-8.64 (m, 1 H), | 980.6 |
| | 8.30-8.38 (m, 1 H), 8.17 (d, J = 8.63 Hz, 1 H), 8.03 (d, J = 8.38 | |
| | Hz, 1 H), 7.70 (d, J = 7.00 Hz, 1 H), 7.64 (t, J = 8.07 Hz, 1 H), 7.51- | |
| | 7.57 (m, 1 H), 7.38 (d, J = 7.50 Hz, 1 H), 7.14 (d, J = 8.76 Hz, 1 | |
| | H), 4.57 (dd, J = 7.88, 4.75 Hz, 1 H), 4.51 (dd, J = 8.82, 5.07 Hz, 1 | |
| | H), 4.37 (dd, J = 7.94, 4.44 Hz, 1 H), 3.75 (t, J = 6.07 Hz, 2 H), | |
| | 3.62 (s, 8 H), 3.57-3.61 (m, 4 H), 3.50-3.56 (m, 4 H), 3.34- | |
| | 3.41 (m, 4 H), 3.19-3.26 (m, 1 H), 2.94 (dd, J = 12.94, 4.94 Hz, 1 | |
| | H), 2.73 (d, J = 12.88 Hz, 1 H), 2.50 (t, J = 6.07 Hz, 2 H), 2.22 (t, | |
| | J = 7.38 Hz, 2 H), 1.83-2.04 (m, 4 H), 1.55-1.74 (m, 4 H), 1.32- | |
| | 1.47 (m, 4 H), 0.91 (m, 9 H). | |
| BF021 | 1H NMR (400 MHz, METHANOL-d4) δ = 8.65 (d, J = 2.0 Hz, | 977.3 |
| | 1H), 8.31 (dd, J = 2.4, 8.7 Hz, 1H), 7.76 (d, J = 7.8 Hz, 1H), 7.66 | |
| | (t, J = 1.9 Hz, 1H), 7.56 (t, J = 7.9 Hz, 1H), 7.38-7.34 (m, 1H), | |
| | 7.10 (d, J = 8.8 Hz, 1H), 4.52 (td, J = 5.2, 8.4 Hz, 2H), 4.34 (dd, | |
| | J = 4.4, 7.9 Hz, 1H), 3.68-3.56 (m, 28H), 3.55-3.51 (m, 2H), | |
| | 3.38-3.33 (m, 2H), 3.25-3.19 (m, 1H), 2.94 (dd, J = 5.0, 12.9 | |
| | Hz, 1H), 2.72 (d, J = 12.8 Hz, 1H), 2.22 (t, J = 7.4 Hz, 2H), 2.02- | |
| | 1.91 (m, 1H), 1.88-1.77 (m, 1H), 1.77-1.52 (m, 4H), 1.48- | |
| | 1.29 (m, 4H), 0.93 (s, 9H) | |
| BF023 | 1HNMR (400 MHz, METHANOL-d4)δ ppm 8.66 (d, J = 2.25 Hz, 1 | 933.6 |
| | H) 8.33(dd, J = 8.69, 2.44 Hz, 1H) 7.77 (d, J = 7.75 Hz, 1H) 7.67 (t, | |
| | J = 1.88 Hz, 1H) 7.56 (t, J = 7.94 Hz, 1H) 7.37 (dd, J = 8.13, 1.63 | |
| | Hz, 1 H) 7.11 (d, J = 8.63 Hz, 1 H) 4.54 (ddd, J = 18.07, 8.32, 5.00 | |
| | Hz, 2 H) 4.37 (dd, J = 7.94, 4.57 Hz, 1 H) 3.58-3.69 (m, 24 H) | |
| | 3.53 (t, J = 5.44 Hz, 2 H) 3.34-3.39 (m, 2 H) 3.18-3.27 (m, 1 H) | |
| | 2.95 (dd, J = 12.88, 5.00 Hz, 1 H) 2.74 (d, J = 12.88 Hz, 1 H) 2.23 | |
| | (t, J = 7.38 Hz, 2 H) 1.92-2.02 (m, 1 H) 1.75-1.88 (m, 1 H) 1.53- | |
| | 1.75 (m, 4 H) 1.31-1.49 (m, 4 H) 0.93 (s, 9 H) | |
| BF026 | 1H NMR (400 MHz, METHANOL-d4) δ = 8.73-8.67 (m, 1H), | 994.5 |
| | 8.39-8.31 (m, 1H), 7.37 (t, J = 8.2 Hz, 1H), 7.06 (dd, J = 2.7, | |
| | 8.7 Hz, 1H), 6.90 (br d, J = 8.8 Hz, 1H), 6.84-6.80 (m, 1H), 6.78 | |
| | (br d, J = 8.2 Hz, 1H), 4.58-4.49 (m, 2H), 4.39-4.32 (m, 1H), | |
| | 4.18-4.11 (m, 2H), 3.87-3.82 (m, 2H), 3.71-3.68 (m, 2H), | |
| | 3.67-3.58 (m, 26H), 3.56-3.50 (m, 2H), 3.39-3.34 (m, 2H), | |
| | 3.27-3.17 (m, 1H), 2.94 (dd, J = 5.0, 12.8 Hz, 1H), 2.73 (d, J = | |
| | 13.0 Hz, 1H), 2.23 (t, J = 7.4 Hz, 2H), 2.03-1.89 (m, 1H), 1.88- | |
| | 1.78 (m, 1H), 1.77-1.53 (m, 4H), 1.50-1.29 (m, 4H), 0.93 (s, | |
| | 9H). | |
| BF028 | 1H NMR (400 MHz, METHANOL-d4) δ = 8.56 (d, J = 2.1 Hz, | 1024.4 |
| | 1H), 8.29 (dd, J = 2.5, 8.6 Hz, 1H), 8.15 (d, J = 8.6 Hz, 1H), 8.01 | |
| | (d, J = 8.5 Hz, 1H), 7.69 (dd, J = 1.0, 7.0 Hz, 1H), 7.65-7.59 (m, | |
| | 1H), 7.53 (dd, J = 7.1, 8.5 Hz, 1H), 7.35 (d, J = 7.0 Hz, 1H), 7.14 | |
| | (d, J = 8.6 Hz, 1H), 4.55-4.44 (m, 2H), 4.29 (dd, J = 4.5, 7.9 Hz, | |
| | 1H), 3.74 (t, J = 6.1 Hz, 2H), 3.67-3.57 (m, 17H), 3.56-3.48 | |
| | (m, 4H), 3.40-3.34 (m, 4H), 3.22-3.15 (m, 1H), 2.91 (dd, J = | |
| | 4.9, 12.8 Hz, 1H), 2.73-2.63 (m, 1H), 2.48 (t, J = 6.0 Hz, 2H), | |
| | 2.20 (t, J = 7.3 Hz, 2H), 1.95-1.78 (m, 4H), 1.74-1.52 (m, 4H), | |
| | 1.44-1.24 (m, 4H), 0.91 (s, 9H). | |

### BF091

### (2S)-2-[[6-[3-[2-[2-[1-[2-[[1-[1-[2-[5-[7-chloro-8-[[(1R)-1-(5-cyano-2-fluoro- phenyl)ethyl]amino]-3-fluoro-6-methyl-1,5-naphthyridin-2-yl]pyrimidin-2-yl]oxyacetyl]azetidin-3-yl]triazol-4-yl]methoxy]ethyl]azetidin-3-yl]oxyethylamino]-2-oxo-ethoxy]phenoxy]pyridine-3-carbonyl]amino]-5,5-dimethyl-hexanoic acid

¹H NMR (400 MHz, METHANOL-d4) δ=8.98 (br d, J=4.9 Hz, 2H), 8.61 (d, J=2.1 Hz, 1H), 8.25 (dd, J=2.4, 8.6 Hz, 1H), 8.19 (br s, 1H), 7.91 (d, J=11.5 Hz, 1H), 7.78 (dd, J=2.0, 6.9 Hz, 1H), 7.64-7.56 (m, 1H), 7.33 (t, J=8.2 Hz, 1H), 7.18 (t, J=9.4 Hz, 1H), 6.98 (d, J=8.7 Hz, 1H), 6.86 (dd, J=2.2, 8.1 Hz, 1H), 6.80-6.72 (m, 2H), 6.45-6.35 (m, 1H), 5.72-5.54 (m, 1H), 5.09 (d, J=4.9 Hz, 2H), 5.06-4.89 (m, 3H), 4.64 (s, 3H), 4.52 (s, 2H), 4.45 (br dd, J=5.3, 7.8 Hz, 2H), 4.31 (br s, 3H), 4.00-3.87 (m, 2H), 3.72 (br s, 2H), 3.52-3.47 (m, 2H), 3.44 (br d, J=4.6 Hz, 2H), 3.37 (br s, 2H), 2.71 (s, 3H), 2.01-1.90 (m, 1H), 1.85-1.76 (m, 1H), 1.71 (d, J=6.7 Hz, 3H), 1.36-1.30 (m, 2H), 0.89 (s, 9H) LC/MS [M+1]: 1201.3

### BF092

### (2S)-2-[[6-[3-[2-[2-[[1-[2-[1-[[1-[2-[5-[7-chloro-8-[[(1R)-1-(5-cyano-2-fluoro- phenyl)ethyl]amino]-3-fluoro-6-methyl-1,5-naphthyridin-2-yl]pyrimidin-2-yl]oxyethyl]azetidin-3-yl]triazol-4-yl]methoxy]ethyl]azetidin-3-yl]oxyethylamino]-2-oxo-ethoxy]phenoxy]pyridine-3-carbonyl]amino]-5,5-dimethyl-hexanoic acid

¹H NMR (400 MHz, METHANOL-d4) δ=9.01 (s, 2H), 8.63 (d, J=2.4 Hz, 1H), 8.26 (dd, J=2.4, 8.7 Hz, 1H), 8.21 (s, 1H), 7.92 (d, J=11.6 Hz, 1H), 7.78 (dd, J=1.7, 6.9 Hz, 1H), 7.64-7.58 (m, 1H), 7.34 (t, J=8.2 Hz, 1H), 7.18 (dd, J=8.6, 10.1 Hz, 1H), 6.99 (d, J=8.6 Hz, 1H), 6.86 (dd, J=2.1, 8.4 Hz, 1H), 6.80-6.72 (m, 2H), 6.44 (q, J=6.9 Hz, 1H), 5.34 (quin, J=6.5 Hz, 1H), 4.77 (s, 2H), 4.64-4.57 (m, 4H), 4.52 (s, 2H), 4.43 (dd, J=5.0, 7.6 Hz, 1H), 4.30-4.19 (m, 3H), 4.07-4.00 (m, 2H), 3.87-3.76 (m, 4H), 3.71-3.65 (m, 2H), 3.45 (br dd, J=4.0, 12.8 Hz, 4H), 3.13 (br t, J=5.1 Hz, 2H), 2.71 (s, 3H), 2.01-1.90 (m, 1H), 1.84-1.74 (m, 1H), 1.72 (d, J=6.8 Hz, 3H), 1.36-1.27 (m, 2H), 0.89 (s, 9H). LC/MS [M+1]: 1187.3

### BF093

### (2S)-2-[[6-[3-[2-[2-[1-[2-[1-[2-[2-[5-[7-chloro-8-[[(1R)-1-(5-cyano-2-fluoro- phenyl)ethyl]amino]-3-fluoro-6-methyl-1,5-naphthyridin-2-yl]pyrimidin-2-yl]oxyethoxy]ethyl]triazol-4-yl]methoxy]ethyl]azetidin-3-yl]oxyethylamino]-2-oxo-ethoxy]phenoxy]pyridine-3-carbonyl]amino]-5,5-dimethyl-hexanoic acid

¹H NMR (400 MHz, METHANOL-d4) δ=9.00 (d, J=1.0 Hz, 2H), 8.61 (d, J=2.3 Hz, 1H), 8.25 (dd, J=2.5, 8.6 Hz, 1H), 8.04 (s, 1H), 7.91 (d, J=11.5 Hz, 1H), 7.79 (dd, J=2.0, 7.0 Hz, 1H), 7.61 (ddd, J=2.2, 4.8, 8.4 Hz, 1H), 7.33 (t, J=8.2 Hz, 1H), 7.17 (dd, J=8.6, 10.1 Hz, 1H), 6.98 (d, J=8.7 Hz, 1H), 6.85 (dd, J=1.8, 8.4 Hz, 1H), 6.80-6.72 (m, 2H), 6.43 (q, J=6.8 Hz, 1H), 4.65-4.58 (m, 6H), 4.51 (s, 2H), 4.45 (dd, J=5.1, 7.8 Hz, 1H), 4.36-4.25 (m, 3H), 3.97 (t, J=5.0 Hz, 2H), 3.96-3.87 (m, 4H), 3.73-3.67 (m, 2H), 3.51-3.46 (m, 2H), 3.45-3.41 (m, 2H), 3.40-3.36 (m, 2H), 2.70 (s, 3H), 2.01-1.91 (m, 1H), 1.84-1.75 (m, 1H), 1.72 (d, J=6.8 Hz, 3H), 1.35-1.28 (m, 2H), 0.89 (s, 9H) LC/MS [M+1]: 1176.2

### BF097

### (2S)-2-[[6-[3-[2-[2-[2-[3-[[2-[(2R)-4-[5-[1-[(2-cyano-3-pyridyl)methyl]-2,2-dimethyl-3-oxo- pyrrolo[2,3-b]pyridin-6-yl]pyrimidin-2-yl]-2-methyl-piperazin-1-yl]-2-oxo-ethyl]amino]-3-oxo-propoxy]ethoxy]ethoxy]ethylcarbamoyl]phenoxy]pyridine-3-carbonyl]amino]-5,5-dimethyl-hexanoic acid

¹H NMR (400 MHz, METHANOL-d₄) δ=8.94 (s, 2H), 8.64-8.58 (m, 2H), 8.26 (dd, J=2.4, 8.6 Hz, 1H), 8.03 (d, J=7.8 Hz, 1H), 7.95 (d, J=8.0 Hz, 1H), 7.74 (d, J=7.6 Hz, 1H), 7.61 (dd, J=4.8, 8.0 Hz, 1H), 7.53 (t, J=7.9 Hz, 1H), 7.35-7.28 (m, 2H), 7.07 (d, J=8.6 Hz, 1H), 5.12 (s, 2H), 4.77-4.60 (m, 3H), 4.51 (dd, J=5.1, 8.9 Hz, 1H), 4.43-4.11 (m, 2H), 4.10-3.96 (m, 1H), 3.71 (t, J=6.0 Hz, 2H), 3.68-3.60 (m, 8H), 3.60-3.53 (m, 4H), 3.51-3.38 (m, 1H), 3.28-3.00 (m, 2H), 2.50 (t, J=6.1 Hz, 2H), 2.02-1.90 (m, 1H), 1.88-1.74 (m, 1H), 1.38-1.29 (m, 8H), 1.27-1.10 (m, 3H), 0.91 (s, 9H).

LC/MS [M+1]: 1097.4

### BF099

### (2S)-2-[[6-[3-[2-[2-[2-[2-[2-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-2-oxo-ethoxy]ethoxy]ethoxy]ethoxy]ethylcarbamoyl]phenoxy]pyridine-3-carbonyl]amino]-5,5-dimethyl-hexanoic acid

¹H NMR (400 MHz, METHANOL-d4) δ=8.94 (s, 2H), 8.64-8.59 (m, 2H), 8.55 (br t, J=5.0 Hz, 1H), 8.28-8.24 (m, 1H), 8.07-8.02 (m, 1H), 7.94 (d, J=8.1 Hz, 1H), 7.74 (d, J=8.0 Hz, 1H), 7.68-7.59 (m, 2H), 7.53 (t, J=8.0 Hz, 1H), 7.35-7.26 (m, 2H), 7.07 (d, J=8.7 Hz, 1H), 5.12 (s, 2H), 4.76-4.57 (m, 4H), 4.54-4.46 (m, 1H), 4.39-4.17 (m, 2H), 4.12-4.08 (m, 1H), 4.03 (s, 2H), 3.80-3.74 (m, 1H), 3.72-3.67 (m, 2H), 3.65-3.60 (m, 11H), 3.59-3.54 (m, 2H), 3.53-3.42 (m, 1H), 3.34 (br d, J=4.4 Hz, 1H), 3.29-3.18 (m, 1H), 3.10-2.99 (m, 1H), 2.01-1.90 (m, 1H), 1.87-1.74 (m, 1H), 1.40-1.29 (m, 8H), 1.27-1.08 (m, 3H), 0.91 (s, 9H) LC/MS [M+1]: 1127.5

### BF101

### (2S)-2-[[6-[3-[2-[2-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]ethoxy]ethoxy]phenoxy]pyridine-3-carbonyl]amino]-5,5-dimethyl-hexanoic acid

¹H NMR (400 MHz, METHANOL-d4) δ=8.95 (s, 2H), 8.62 (d, J=2.8 Hz, 2H), 8.23 (dd, J=2.5, 8.6 Hz, 1H), 8.03 (d, J=7.4 Hz, 1H), 7.95 (d, J=8.0 Hz, 2H), 7.62 (dd, J=4.7, 8.1 Hz, 1H), 7.34-7.29 (m, 2H), 6.97 (d, J=8.5 Hz, 1H), 6.84 (dd, J=1.7, 8.3 Hz, 1H), 6.77-6.70 (m, 2H), 5.66-5.36 (m, 2H), 5.12 (s, 2H), 4.75-4.69 (m, 2H), 4.65 (s, 2H), 4.60-4.54 (m, 2H), 4.49 (dd, J=5.3, 8.5 Hz, 1H), 4.35 (br dd, J=3.5, 8.3 Hz, 1H), 4.15-4.06 (m, 2H), 3.84-3.80 (m, 2H), 3.73-3.57 (m, 14H), 3.16-3.07 (m, 1H), 2.02-1.90 (m, 1H), 1.85-1.76 (m, 1H), 1.42-1.26 (m, 11H), 0.90 (s, 9H) LC/MS [M+1]: 1124.5

### BF102

### (2S)-2-[[6-[3-[2-[2-[2-[2-[2-[[1]-[2-[(2R)-4-[5-[1-[(2-cyano-3-pyridyl)methyl]-2,2- dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxy]ethoxy]ethoxy]ethoxy]phenoxy]pyridine-3-carbonyl]amino]-5,5-dimethyl-hexanoic acid

¹H NMR (400 MHz, METHANOL-d4) δ=8.93 (s, 2H), 8.64-8.58 (m, 2H), 8.21 (dd, J=2.3, 8.6 Hz, 1H), 8.02 (dd, J=1.4, 8.1 Hz, 1H), 7.93 (d, J=8.1 Hz, 1H), 7.60 (dd, J=4.8, 8.1 Hz, 1H), 7.34-7.25 (m, 2H), 6.95 (d, J=8.6 Hz, 1H), 6.84 (dd, J=2.1, 8.0 Hz, 1H), 6.76 (t, J=2.3 Hz, 1H), 6.70 (dd, J=1.7, 8.0 Hz, 1H), 5.11 (s, 2H), 4.82-4.55 (m, 3H), 4.50 (dd, J=5.2, 8.6 Hz, 1H), 4.43-4.20 (m, 2H), 4.12 (br dd, J=3.8, 5.4 Hz, 2H), 4.05 (s, 2H), 3.83 (dd, J=3.9, 5.3 Hz, 2H), 3.75-3.71 (m, 2H), 3.70-3.58 (m, 14H), 3.54-3.39 (m, 1H), 3.33 (br d, J=1.7 Hz, 1H), 3.28-3.15 (m, 1H), 3.15-2.99 (m, 1H), 2.02-1.90 (m, 1H), 1.88-1.74 (m, 1H), 1.42-1.28 (m, 8H), 1.27-1.08 (m, 3H), 0.91 (s, 9H) LC/MS [M+1]: 1144.5

### BF103

### (2S)-2-[[6-[3-[10-[4-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-1-yl]decylcarbamoyl]phenoxy]pyridine-3-carbonyl]amino]-5,5-dimethyl-hexanoic acid

1H NMR (400 MHz, METHANOL-d4) δ=8.94 (s, 2H), 8.64-8.58 (m, 2H), 8.26 (dd, J=2.4, 8.6 Hz, 1H), 8.02 (dd, J=1.3, 8.2 Hz, 1H), 7.97-7.89 (m, 2H), 7.69 (d, J=8.0 Hz, 1H), 7.63-7.58 (m, 2H), 7.52 (t, J=7.9 Hz, 1H), 7.33-7.26 (m, 2H), 7.07 (d, J=8.7 Hz, 1H), 5.11 (s, 2H), 4.79-4.72 (m, 1H), 4.70-4.55 (m, 2H), 4.54-4.44 (m, 2H), 4.41 (br t, J=6.3 Hz, 2H), 4.05-3.85 (m, 2.5H), 3.54-3.46 (m, 0.5H), 3.35-3.32 (m, 2H), 3.22-3.01 (m, 2H), 1.95-1.76 (m, 4H), 1.60-1.54 (m, 2H), 1.36-1.25 (m, 20H), 1.23-1.11 (m, 3H), 0.91 (s, 9H) LC/MS [M+1]: 1101.5

### BF105

### (2S)-2-[[6-[3-[5-[2-[3-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-3-oxo-propoxy]ethoxy]pentylcarbamoyl]phenoxy]pyridine-3-carbonyl]amino]-5,5-dimethyl-hexanoic acid

¹H NMR (400 MHz, METHANOL-d4) δ=8.93 (s, 2H), 8.64-8.58 (m, 2H), 8.54-8.47 (m, 1H), 8.25 (dd, J=2.4, 8.6 Hz, 1H), 8.03 (d, J=7.5 Hz, 1H), 7.94 (d, J=8.1 Hz, 1H), 7.71 (d, J=7.9 Hz, 1H), 7.64-7.57 (m, 2H), 7.51 (t, J=8.0 Hz, 1H), 7.34-7.26 (m, 2H), 7.06 (d, J=8.7 Hz, 1H), 5.12 (s, 2H), 4.73-4.60 (m, 4H), 4.55-4.47 (m, 1H), 4.42-4.31 (m, 0.5H), 4.28-3.98 (m, 2.5H), 3.74 (t, J=6.1 Hz, 2H), 3.62-3.55 (m, 4H), 3.48 (br t, J=6.3 Hz, 2H), 3.39-3.34 (m, 2H), 3.29-2.98 (m, 2H), 2.52 (t, J=6.0 Hz, 2H), 2.00-1.91 (m, 1H), 1.86-1.76 (m, 1H), 1.62 (qd, J=7.4, 14.8 Hz, 4H), 1.47-1.40 (m, 2H), 1.38-1.30 (m, 8H), 1.26-1.08 (m, 3H), 0.91 (s, 9H) LC/MS [M+1]: 1095.6

### BF107

### (2S)-2-[[6-[3-[3-[2-[5-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-5-oxo-pentoxy]ethoxy]propylcarbamoyl]phenoxy]pyridine-3-carbonyl]amino]-5,5-dimethyl-hexanoic acid

1H NMR (400 MHz, METHANOL-d4) δ=8.94 (s, 2H), 8.63-8.60 (m, 2H), 8.29-8.24 (m, 1H), 8.05-8.01 (m, 1H), 7.94 (d, J=8.0 Hz, 1H), 7.70 (s, 1H), 7.63-7.59 (m, 2H), 7.53 (s, 1H), 7.30 (br d, J=8.0 Hz, 2H), 7.07 (d, J=8.6 Hz, 1H), 5.12 (s, 2H), 4.76-4.56 (m, 3H), 4.54-4.47 (m, 1H), 4.40-4.19 (m, 1H), 4.17-3.95 (m, 2H), 3.86-3.66 (m, 1H), 3.65-3.41 (m, 12H), 3.27-3.12 (m, 1H), 3.11-2.98 (m, 1H), 2.29 (t, J=7.4 Hz, 2H), 1.88 (br t, J=6.3 Hz, 2H), 1.68 (br d, J=7.9 Hz, 2H), 1.59 (br d, J=7.8 Hz, 2H), 1.38-1.32 (m, 8H), 1.26-1.11 (m, 3H), 0.91 (s, 9H) LC/MS [M+1]: 1095.6

### BF109

### (2S)-2-[[6-[3-[1-[2-[2-[4-[[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-2-oxo-ethyl]piperazin-1-yl]ethoxy]ethyl]triazol-4-yl]phenoxy]pyridine-3-carbonyl]amino]-5,5-dimethyl-hexanoic acid

¹H NMR (400 MHz, DMSO-d6) δ=8.94 (s, 2H), 8.70-8.63 (m, 3H), 8.58 (s, 1H), 8.31 (dd, J=2.4, 8.6 Hz, 1H), 8.00-7.92 (m, 2H), 7.81 (br t, J=4.8 Hz, 1H), 7.74 (d, J=7.7 Hz, 1H), 7.67 (dd, J=4.6, 8.1 Hz, 1H), 7.62 (d, J=1.7 Hz, 1H), 7.51 (t, J=7.9 Hz, 1H), 7.37 (d, J=8.1 Hz, 1H), 7.19-7.12 (m, 2H), 5.04 (s, 2H), 4.78-4.42 (m, 5H), 4.36-4.00 (m, 3.5H), 3.98-3.86 (m, 3.5H), 3.83 (br t, J=5.0 Hz, 3.5H), 3.78-3.67 (m, 1H), 3.52 (br t, J=5.6 Hz, 2H), 3.25 (br s, 2H), 3.01-2.92 (m, 1H), 2.87 (s, 2H), 2.47-2.33 (m, 8H), 1.84-1.68 (m, 2H), 1.36-1.20 (m, 8H), 1.16-0.99 (m, 3H), 0.86 (s, 9H) LC/MS [M+1]: 1131.3

### BF110

### (2S)-2-[[6-[3-[1]-[2-[2-[4-[[1]-[2-(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methyl]piperazin-1-yl]-2-oxo-ethoxy]ethyl]triazol-4-yl]phenoxy]pyridine-3-carbonyl]amino]-5,5-dimethyl-hexanoic acid

1H NMR (400 MHz, DMSO-d6) δ=8.96 (s, 2H), 8.69-8.66 (m, 2H), 8.64 (br d, J=2.1 Hz, 2H), 8.30 (dd, J=2.3, 8.6 Hz, 1H), 7.99-7.93 (m, 2H), 7.88-7.82 (m, 1H), 7.74 (d, J=8.1 Hz, 1H), 7.68 (dd, J=4.6, 8.2 Hz, 1H), 7.63 (s, 1H), 7.52 (t, J=7.9 Hz, 1H), 7.38 (d, J=8.0 Hz, 1H), 7.18-7.11 (m, 2H), 5.71-5.30 (m, 2H), 5.05 (s, 2H), 4.73-4.63 (m, 0.5H), 4.59 (br t, J=4.8 Hz, 3H), 4.54-4.46 (m, 1H), 4.41-4.21 (m, 2H), 4.17 (s, 2H), 3.89 (br t, J=5.0 Hz, 2.5H), 3.53 (s, 2H), 3.26 (br s, 8H), 3.05-2.96 (m, 1H), 2.31 (br d, J=2.6 Hz, 2H), 1.83-1.65 (m, 2H), 1.36-1.22 (m, 8H), 1.21-1.05 (m, 3H), 0.86 (s, 9H). LC/MS [M+1]: 1169.3

### BF115

### (2S)-2-[[6-[3-[2-[2-[2-[[1-[2-[1-[2-[(2R)-4-[5-[1-[(2-cyano-3-pyridyl)methyl]-2,2- dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethyl]azetidin-3-yl]oxyethoxy]ethoxy]ethoxy]phenoxy]pyridine-3-carbonyl]amino]-5,5-dimethyl-hexanoic acid (2S)-2-[[6-[3-[2-[2-[2-[[1-[2-[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethylJazetidin-3-yl]oxyethoxy]ethoxy]ethoxy]phenoxy]pyridine-3-carbonyl]amino]-5,5-dimethyl-hexanoic acid

¹H NMR (400 MHz, METHANOL-d4) δ ppm 8.95 (s, 2H) 8.59-8.65 (m, 2H) 8.25 (dd, J=8.70, 2.50 Hz, 1H) 7.98-8.05 (m, 2H) 7.93-7.97 (m, 1H) 7.61 (dd, J=8.11, 4.65 Hz, 1H) 7.28-7.35 (m, 2H) 6.99 (d, J=8.82 Hz, 1H) 6.84 (dd, J=8.11, 1.79 Hz, 1H) 6.70-6.78 (m, 2H) 5.56-5.75 (m, 1H) 5.36-5.50 (m, 1H) 5.11 (s, 2H) 4.65-4.82 (m, 4H) 4.63 (s, 2H) 4.45 (dd, J=7.63, 5.01 Hz, 1H) 4.25-4.38 (m, 4H) 4.14 (dd, J=5.36, 3.70 Hz, 2H) 3.95 (br dd, J=11.68, 4.65 Hz, 2H) 3.85-3.92 (m, 1H) 3.82 (br dd, J=5.30, 3.64 Hz, 2H) 3.57-3.70 (m, 12H) 3.37 (br d, J=3.34 Hz, 1H) 1.90-2.03 (m, 1H) 1.73-1.84 (m, 1H) 1.37 (s, 6H) 1.29-1.35 (m, 3.5H) 1.15 (br d, J=7.15 Hz, 1.5H) 0.89 (s, 9H). LC/MS [M+1]: 1179.3

### BF116

### (2S)-2-[[6-[3-[2-[2-[2-[2-[2-[(2R)-4-[5-[1-[(2-cyano-3-pyridyl)methyl]-2,2-dimethyl-3-oxo- pyrrolo[2,3-b]pyridin-6-yl]pyrimidin-2-yl]-2-methyl-piperazin-1-yl]-2-oxo-ethoxy]ethoxy]ethoxy]ethylamino]-2-oxo-ethoxy]phenoxy]pyridine-3-carbonyl]amino]-5,5-dimethyl-hexanoic acid

¹H NMR (400 MHz, METHANOL-d₄) δ=8.92 (s, 2H), 8.61 (d, J=2.6 Hz, 2H), 8.23 (dd, J=2.4, 8.7 Hz, 1H), 8.16-8.10 (m, 1H), 8.02 (d, J=8.1 Hz, 1H), 7.93 (d, J=8.0 Hz, 1H), 7.60 (dd, J=4.7, 8.1 Hz, 1H), 7.34 (t, J=8.2 Hz, 1H), 7.27 (d, J=8.1 Hz, 1H), 6.99 (d, J=8.6 Hz, 1H), 6.87 (dd, J=2.0, 8.3 Hz, 1H), 6.83-6.75 (m, 2H), 5.11 (s, 2H), 4.75-4.56 (m, 2H), 4.55-4.47 (m, 3H), 4.40-4.11 (m, 3H), 3.86 (br d, J=12.9 Hz, 1H), 3.65 (s, 4H), 3.62-3.54 (m, 6H), 3.50-3.37 (m, 3H), 3.29-3.16 (m, 1H), 3.02 (br d, J=8.1 Hz, 1H), 2.02-1.90 (m, 1H), 1.87-1.75 (m, 1H), 1.42-1.28 (m, 8H), 1.21 (br s, 1H), 1.12 (br d, J=5.4 Hz, 2H), 0.91 (s, 9H). LC/MS [M+1]: 1056.4

### BF117

### (2S)-2-[[6-[3-[2-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethylamino]-2-oxo-ethoxy]phenoxy]pyridine-3-carbonyl]amino]-5,5-dimethyl-hexanoic acid

¹H NMR (400 MHz, METHANOL-d₄) δ=8.93 (s, 2H), 8.64-8.58 (m, 2H), 8.24 (dd, J=2.5, 8.6 Hz, 1H), 8.02 (dd, J=1.1, 8.2 Hz, 1H), 7.93 (d, J=8.0 Hz, 1H), 7.60 (dd, J=4.7, 8.2 Hz, 1H), 7.35 (t, J=8.2 Hz, 1H), 7.28 (d, J=8.0 Hz, 1H), 7.00 (d, J=8.7 Hz, 1H), 6.88 (dd, J=2.3, 8.3 Hz, 1H), 6.83-6.75 (m, 2H), 5.11 (s, 2H), 4.73-4.59 (m, 2H), 4.54-4.46 (m, 3H), 4.40-4.13 (m, 3H), 3.89-3.82 (m, 0.5H), 3.69-3.51 (m, 15H), 3.47-3.41 (m, 3H), 3.28-3.17 (m, 1H), 3.09-2.94 (m, 0.5H), 2.02-1.91 (m, 1H), 1.86-1.75 (m, 1H), 1.38-1.30 (m, 8H), 1.24-1.11 (m, 3H), 0.91 (s, 9H). LC/MS [M+1]: 1100.3

### BF118

### (2S)-2-[[6-[3-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethylamino]-2-oxo-ethoxy]phenoxy]pyridine-3-carbonyl]amino]-5,5-dimethyl-hexanoic acid

¹H NMR (400 MHz, METHANOL-d₄) δ=8.92 (s, 2H), 8.61 (br s, 2H), 8.25-8.15 (m, 2H), 8.06-8.00 (m, 1H), 7.94 (d, J=8.1 Hz, 1H), 7.61 (dd, J=4.7, 8.1 Hz, 1H), 7.37-7.25 (m, 2H), 6.98 (d, J=8.6 Hz, 1H), 6.88 (br d, J=8.6 Hz, 1H), 6.84-6.75 (m, 2H), 5.12 (s, 2H), 4.80-4.61 (m, 2H), 4.59-4.46 (m, 4H), 4.41-4.13 (m, 3H), 3.86-3.79 (m, 0.5H), 3.65 (s, 4H), 3.62-3.58 (m, 2H), 3.48 (q, J=5.1 Hz, 2H), 3.43-3.35 (m, 1H), 3.30-3.16 (m, 1H), 3.07-2.97 (m, 0.5H), 2.00-1.90 (m, 1H), 1.86-1.75 (m, 1H), 1.39-1.19 (m, 9H), 1.12 (br d, J=6.4 Hz, 2H), 0.91 (s, 9H). LC/MS [M+1]: 1012.2

### BF120

### (2S)-2-[[6-[3-[1-[2-[1-[4-[[2-[(2R)-4-[5-[1-[(2-cyano-3-pyridyl)methyl]-2,2-dimethyl-3-oxo- pyrrolo[2,3-b]pyridin-6-yl]pyrimidin-2-yl]-2-methyl-piperazin-1-yl]-2-oxo-ethyl]amino]-4-oxo-butyl]azetidin-3-yl]oxyethyl]triazol-4-yl]phenoxy]pyridine-3-carbonyl]amino]-5,5-dimethyl-hexanoic acid

(400 MHz, METHANOL-d4) δ ppm 0.89 (s, 9H) 1.11 (br d, J=6.11 Hz, 2H) 1.19-1.26 (m, 2H) 1.27-1.33 (m, 2H) 1.36 (s, 6H) 1.74-1.84 (m, 3H) 1.89-2.03 (m, 1H) 2.30-2.39 (m, 2H) 2.95-3.07 (m, 1H) 3.13 (br t, J=7.09 Hz, 2H) 3.34-3.50 (m, 2H) 3.77-3.85 (m, 2H) 3.94 (br t, J=4.65 Hz, 2H) 3.97-4.17 (m, 2H) 4.17-4.29 (m, 3H) 4.33-4.40 (m, 1H) 4.42-4.48 (m, 1H) 4.54-4.71 (m, 5H) 5.11 (s, 2H) 7.06 (d, J=8.56 Hz, 1H) 7.15 (br d, J=6.85 Hz, 1H) 7.30 (d, J=8.07 Hz, 1H) 7.47-7.55 (m, 1H) 7.58-7.65 (m, 2H) 7.76 (br d, J=7.58 Hz, 1H) 7.95 (d, J=8.07 Hz, 1H) 7.99-8.06 (m, 1H) 8.24-8.31 (m, 1H) 8.40 (s, 1H) 8.60-8.66 (m, 2H) 8.93 (s, 2H). LC/MS [M+1]: 1102.3

### BF122

### (2S)-2-[[6-[3-[[1-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]acetyl]-4-piperidyl]oxy]phenoxy]pyridine-3-carbonyl]amino]-5,5-dimethyl-hexanoic acid

¹H NMR (400 MHz, METHANOL-d4) δ=8.95 (s, 2H), 8.65-8.61 (m, 2H), 8.23 (dd, J=2.4, 8.6 Hz, 1H), 8.06-8.00 (m, 1H), 8.00-7.92 (m, 2H), 7.61 (dd, J=4.6, 8.1 Hz, 1H), 7.36-7.28 (m, 2H), 6.99 (d, J=8.7 Hz, 1H), 6.87 (dd, J=1.9, 8.5 Hz, 1H), 6.80 (t, J=2.2 Hz, 1H), 6.72 (dd, J=1.4, 8.1 Hz, 1H), 5.67-5.50 (m, 1H), 5.47-5.34 (m, 1H), 5.12 (s, 2H), 4.83-4.68 (m, 3H), 4.68-4.54 (m, 4H), 4.54-4.46 (m, 1H), 4.36-4.31 (m, 1H), 4.29-4.19 (m, 2H), 3.89-3.63 (m, 6H), 3.61-3.34 (m, 4H), 3.16-3.00 (m, 1H), 2.01-1.89 (m, 3H), 1.85-1.67 (m, 3H), 1.40-1.31 (m, 8H), 1.30-1.28 (m, 2H), 1.16-1.10 (m, 2H), 0.91 (s, 9H). LC/MS [M+1]: 1133.3

### BF123

### (2S)-2-[[6-[3-[[1-[2-[2-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]ethoxycarbonyl]-4-piperidyl]oxy]phenoxy]pyridine-3-carbonyl]amino]-5,5-dimethyl-hexanoic acid

¹H NMR (400 MHz, METHANOL-d4) δ=8.95 (s, 2H), 8.62 (d, J=2.7 Hz, 2H), 8.22 (dd, J=2.4, 8.6 Hz, 1H), 8.02 (d, J=8.2 Hz, 1H), 7.94 (d, J=8.0 Hz, 2H), 7.61 (dd, J=4.6, 8.1 Hz, 1H), 7.36-7.27 (m, 2H), 6.98 (d, J=8.6 Hz, 1H), 6.86 (dd, J=2.1, 8.2 Hz, 1H), 6.78 (t, J=2.2 Hz, 1H), 6.71 (dd, J=2.0, 8.0 Hz, 1H), 5.68-5.51 (m, 1H), 5.45-5.36 (m, 1H), 5.11 (s, 2H), 4.81-4.60 (m, 5H), 4.56 (tt, J=3.7, 7.2 Hz, 1H), 4.50 (dd, J=5.2, 8.8 Hz, 1H), 4.38-4.29 (m, 1H), 4.25-4.17 (m, 2H), 3.90-3.81 (m, 0.5H), 3.78-3.62 (m, 8H), 3.61-3.50 (m, 0.5H), 3.43-3.33 (m, 3H), 3.18-3.00 (m, 1H), 1.99-1.87 (m, 3H), 1.86-1.74 (m, 1H), 1.73-1.61 (m, 2H), 1.40-1.26 (m, 9.5H), 1.14 (br d, J=6.3 Hz, 1.5H), 0.90 (s, 9H). LC/MS [M+1]: 1163.3

### BF124

### (2S)-2-[[6-[3-[[1-[5-[3-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]pyrrolidin-1-yl]-5-oxo-pentanoyl]-4-piperidyl]oxy]phenoxy]pyridine-3-carbonyl]amino]-5,5-dimethyl-hexanoic acid

¹H NMR (400 MHz, METHANOL-d4) δ=8.96 (s, 2H), 8.62 (d, J=2.6 Hz, 2H), 8.27-8.21 (m, 1H), 8.03 (d, J=8.6 Hz, 1H), 7.98-7.92 (m, 2H), 7.61 (dd, J=4.6, 8.1 Hz, 1H), 7.37-7.28 (m, 2H), 6.99 (dd, J=2.3, 8.8 Hz, 1H), 6.88 (br d, J=7.8 Hz, 1H), 6.80 (s, 1H), 6.72 (br d, J=7.8 Hz, 1H), 5.71-5.51 (m, 1H), 5.46-5.30 (m, 1H), 5.16-5.08 (m, 2H), 4.76-4.59 (m, 5H), 4.51 (dd, J=5.3, 8.7 Hz, 1H), 4.40-4.22 (m, 2H), 3.92-3.72 (m, 3H), 3.67-3.39 (m, 8H), 3.18-3.02 (m, 1H), 2.51-2.32 (m, 4H), 2.23-2.08 (m, 1H), 2.04-1.86 (m, 6H), 1.84-1.65 (m, 3H), 1.39-1.30 (m, 9.5H), 1.18-1.14 (m, 1.5H), 0.91 (s, 9H). LC/MS [M+1]: 1214.5

### BF127

### (2S)-2-[[6-[3-[3-[3-[2-[[1-[2-[(2R)-4-[5-[1-[(2-cyano-3-pyridyl)methyl]-2,2-dimethyl-3-oxo- pyrrolo[2,3-b]pyridin-6-yl]pyrimidin-2-yl]-2-methyl-piperazin-1-yl]-2-oxo-ethyl]triazol-4-yl]methoxy]ethoxy]propylsulfonylamino]cyclobutoxy]phenoxy]pyridine-3-carbonyl]amino]-5,5-dimethyl-hexanoic acid

¹H NMR (400 MHz, METHANOL-d4) δ=8.95 (s, 2H), 8.69-8.56 (m, 2H), 8.22 (dd, J=2.4, 8.6 Hz, 1H), 8.03 (d, J=8.5 Hz, 1H), 7.95 (br d, J=7.9 Hz, 2H), 7.61 (dd, J=4.7, 8.2 Hz, 1H), 7.35-7.26 (m, 2H), 6.98 (d, J=8.6 Hz, 1H), 6.71 (dd, J=2.2, 8.2 Hz, 2H), 6.62 (t, J=2.2 Hz, 1H), 5.68-5.50 (m, 1H), 5.46-5.34 (m, 1H), 5.12 (s, 2H), 4.78-4.63 (m, 6H), 4.50 (br dd, J=5.1, 8.7 Hz, 1H), 4.46-4.27 (m, 1H), 4.14-4.08 (m, 1H), 3.93-3.79 (m, 1H), 3.69-3.64 (m, 2H), 3.63-3.50 (m, 6H), 3.37 (br s, 1H), 3.11-3.06 (m, 2H), 2.54-2.47 (m, 2H), 2.09-1.88 (m, 4H), 1.84-1.76 (m, 1H), 1.65-1.58 (m, 1H), 1.36 (s, 8.5H), 1.16 (br d, J=6.8 Hz, 1.5H), 0.91 (s, 9H). LC/MS [M+1]: 1183.3

### BF128

### (2S)-2-[[6-[3-[[1-[3-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]propanoyl]-4-piperidyl]oxy]phenoxy]pyridine-3-carbonyl]amino]-5,5-dimethyl-hexanoic acid

¹H NMR (400 MHz, METHANOL-d4) δ=8.96 (s, 2H), 8.66-8.60 (m, 2H), 8.24 (dd, J=2.3, 8.8 Hz, 1H), 8.05-8.01 (m, 1H), 7.99-7.93 (m, 2H), 7.62 (dd, J=4.7, 8.1 Hz, 1H), 7.36-7.28 (m, 2H), 7.00 (d, J=8.7 Hz, 1H), 6.87 (dd, J=1.8, 7.9 Hz, 1H), 6.80 (s, 1H), 6.72 (br d, J=8.1 Hz, 1H), 5.69-5.52 (m, 1H), 5.48-5.37 (m, 1H), 5.12 (s, 2H), 4.82-4.69 (m, 2H), 4.68-4.55 (m, 4H), 4.50 (br dd, J=5.1, 8.3 Hz, 1H), 4.34 (br dd, J=1.5, 10.8 Hz, 1H), 3.93-3.68 (m, 5H), 3.64-3.44 (m, 3H), 3.15-3.00 (m, 1H), 2.78-2.62 (m, 2H), 1.99-1.86 (m, 3H), 1.85-1.76 (m, 1H), 1.75-1.65 (m, 2H), 1.36 (s, 6H), 1.33-1.27 (m, 3.5H), 1.14 (br d, J=6.6 Hz, 1.5H), 0.90 (s, 9H). LC/MS [M+1]: 1183.3

### BF130

### (2S)-2-[[6-[3-[[1-[1-[4-[[1-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]butanoyl]pyrrolidine-3-carbonyl]-4-piperidyl]oxy]phenoxy]pyridine-3-carbonyl]amino]-5,5-dimethyl-hexanoic acid

¹H NMR (400 MHz, METHANOL-d4) δ=8.95 (s, 2H), 8.61 (br s, 2H), 8.22 (br d, J=8.6 Hz, 1H), 8.05-8.00 (m, 1H), 7.94 (d, J=8.1 Hz, 2H), 7.61 (dd, J=4.7, 8.0 Hz, 1H), 7.35-7.27 (m, 2H), 6.98 (d, J=8.8 Hz, 1H), 6.88 (br d, J=8.6 Hz, 1H), 6.80 (s, 1H), 6.74-6.68 (m, 1H), 5.72-5.53 (m, 1H), 5.51-5.36 (m, 1H), 5.11 (s, 2H), 4.77-4.62 (m, 3H), 4.60 (s, 2H), 4.50 (br dd, J=5.2, 8.5 Hz, 1H), 4.42-4.31 (m, 1H), 3.93-3.77 (m, 3H), 3.76-3.61 (m, 3H), 3.60-3.50 (m, 6H), 3.46-3.36 (m, 2H), 3.18-3.04 (m, 1H), 2.47-2.36 (m, 2H), 2.23-1.88 (m, 7H), 1.86-1.67 (m, 3H), 1.39-1.35 (m, 6H), 1.34-1.26 (m, 3H), 1.20-1.11 (m, 2H), 0.91 (d, J=1.2 Hz, 9H). LC/MS [M+1]: 1214.4

### BF132

### (2S)-2-[[2-[3-[[5-[[1-[2-[2-[[1-[2-[(2R)-4-[5-[1-[(2-cyano-3-pyridyl)methyl]-2,2- dimethyl-3-oxo-pyrrolo[2,3-b]pyridin-6-yl]pyrimidin-2-yl]-2-methyl-piperazin-1-yl]-2-oxo-ethyl]triazol-4-yl]methoxy]ethoxy]acetyl]-4-piperidyl]oxy]-2-pyridyl]oxy]phenoxy]acetyl]amino]-5,5-dimethyl-hexanoic acid

¹H NMR (400 MHz, METHANOL-d4) δ=8.96 (s, 2H), 8.62 (d, J=3.7 Hz, 1H), 8.08-7.91 (m, 3H), 7.87 (d, J=2.8 Hz, 1H), 7.61 (dd, J=4.7, 8.3 Hz, 1H), 7.51 (dd, J=2.9, 8.8 Hz, 1H), 7.34-7.24 (m, 2H), 6.92 (d, J=8.9 Hz, 1H), 6.83-6.64 (m, 3H), 5.69-5.52 (m, 1H), 5.48-5.36 (m, 1H), 5.12 (s, 2H), 4.81-4.52 (m, 8H), 4.44-4.16 (m, 4H), 3.91-3.62 (m, 7H), 3.58-3.38 (m, 3H), 3.18-2.96 (m, 1H), 2.02-1.83 (m, 3H), 1.78-1.65 (m, 3H), 1.40-1.32 (m, 6H), 1.29 (br d, J=3.3 Hz, 1.5H), 1.16 (br dd, J=7.7, 17.2 Hz, 3.5H), 0.84 (s, 9H). LC/MS [M+1]: 1163.5

### BF134

### (2S)-2-[[6-[3-[2-[2-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethoxy]ethoxy]ethylamino]-2-oxo-ethoxy]phenoxy]pyridine-3-carbonyl]amino]-5,5-dimethyl-hexanoic acid

¹H NMR (400 MHz, METHANOL-d4) δ=8.89 (s, 2H), 8.63-8.57 (m, 2H), 8.23 (dd, J=2.4, 8.6 Hz, 1H), 8.01 (dd, J=1.3, 8.1 Hz, 1H), 7.93 (d, J=8.0 Hz, 1H), 7.60 (dd, J=4.6, 8.1 Hz, 1H), 7.34 (t, J=8.2 Hz, 1H), 7.27 (d, J=8.1 Hz, 1H), 6.99 (d, J=8.7 Hz, 1H), 6.87 (dd, J=2.1, 8.3 Hz, 1H), 6.83-6.73 (m, 2H), 5.11 (s, 2H), 4.55-4.47 (m, 3H), 3.66-3.53 (m, 18H), 3.47-3.41 (m, 2H), 2.01-1.91 (m, 1H), 1.87-1.75 (m, 1H), 1.41-1.28 (m, 8H), 0.91 (s, 9H). LC/MS [M+1]: 1003.2

### BF135

### (2S)-2-[[6-[3-[2-[2-[2-[2-[2-[2-[2-[[5-[1-[(2-cyano-3-pyridyl)methyl]-2,2-dimethyl-3-oxo- pyrrolo[2,3-b]pyridin-6-yl]pyrimidin-2-yl]amino]ethoxy]ethoxy]ethoxy]ethoxy]ethoxy]ethylamino]-2-oxoethoxy]phenoxy]pyridine-3-carbonyl]amino]-5,5-dimethyl-hexanoic acid

¹H NMR (400 MHz, METHANOL-d4) δ=8.87 (s, 2H), 8.63-8.58 (m, 2H), 8.24 (dd, J=2.4, 8.7 Hz, 1H), 8.17-8.09 (m, 1H), 8.01 (d, J=7.9 Hz, 1H), 7.93 (d, J=7.9 Hz, 1H), 7.60 (dd, J=4.6, 8.1 Hz, 1H), 7.34 (t, J=8.3 Hz, 1H), 7.26 (d, J=7.9 Hz, 1H), 6.99 (d, J=8.7 Hz, 1H), 6.87 (dd, J=2.0, 8.3 Hz, 1H), 6.83-6.75 (m, 2H), 5.11 (s, 2H), 4.56-4.48 (m, 3H), 3.67-3.53 (m, 21H), 3.45 (q, J=5.4 Hz, 2H), 2.02-1.89 (m, 1H), 1.87-1.74 (m, 1H), 1.43-1.27 (m, 8H), 0.97-0.87 (m, 9H). LC/MS [M+1]: 1047.2

### BF136

### (2S)-2-[[6-[3-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethylamino]-2-oxo-ethoxy]phenoxy]pyridine-3-carbonyl]amino]-5,5-dimethylhexanoic acid

LC/MS [M+1]: 871.4

¹H NMR (400 MHz, METHANOL-d₄) δ=8.73-9.08 (m, 2H) 8.60 (br d, J=3.42 Hz, 2H) 8.21 (dd, J=8.68, 1.96 Hz, 1H) 8.01 (d, J=7.82 Hz, 1H) 7.91 (d, J=8.07 Hz, 1H) 7.59 (dd, J=8.07, 4.65 Hz, 1H) 7.30 (t, J=8.19 Hz, 1H) 7.21 (d, J=8.07 Hz, 1H) 6.96 (d, J=8.56 Hz, 1H) 6.84 (dd, J=8.31, 1.96 Hz, 1H) 6.80 (t, J=2.08 Hz, 1H) 6.74 (dd, J=8.01, 1.77 Hz, 1H) 5.10 (s, 2H) 4.50 (s, 3H) 3.54-3.64 (m, 6H) 3.46 (s, 2H) 1.88-2.03 (m, 1H) 1.73-1.87 (m, 1H) 1.34 (s, 8H) 0.90 (s, 9H)

### BF138

### (2S)-2-[[6-[3-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethylamino]-2-oxo-ethoxy]phenoxy]pyridine-3-carbonyl]amino]-5,5-dimethyl-hexanoic acid

¹H NMR (400 MHz, CHLOROFORM-d) δ=8.68-8.59 (m, 2H), 8.25 (dd, J=2.3, 8.5 Hz, 1H), 8.02-7.86 (m, 3H), 7.47 (dd, J=4.7, 8.1 Hz, 1H), 7.37 (t, J=8.2 Hz, 1H), 7.07 (dd, J=8.3, 16.8 Hz, 3H), 6.89-6.73 (m, 4H), 5.11 (s, 2H), 4.80 (q, J=6.3 Hz, 1H), 4.53 (d, J=2.5 Hz, 2H), 3.82-3.54 (m, 12H), 2.13-2.01 (m, 1H), 1.92-1.75 (m, 1H), 1.33 (d, J=2.0 Hz, 7H), 0.89 (s, 9H). LC/MS [M+1]: 915.4

### BF140

### (S)-2-(6-(3-((17-((5-(1-((2-cyanopyridin-3-yl)methyl)-2,2-dimethyl-3-oxo-2,3-dihydro-1H-pyrrolo[2,3-b]pyridin-6-yl)pyrimidin-2-yl)oxy)-2-oxo-6,9, 12, 15-tetraoxa-3-azaheptadecyl)oxy)phenoxy)nicotinamido)-5,5-dimethylhexanoic acid

¹H NMR (400 MHz, METHANOL-d₄) δ=9.10 (br s, 2H), 8.61 (br s, 2H), 8.23 (dd, J=2.1, 8.5 Hz, 1H), 8.01 (dd, J=8.0, 11.7 Hz, 2H), 7.60 (dd, J=4.8, 8.0 Hz, 1H), 7.39-7.30 (m, 2H), 6.98 (d, J=8.6 Hz, 1H), 6.87 (dd, J=2.1, 8.3 Hz, 1H), 6.82-6.78 (m, 1H), 6.76 (br d, J=8.0 Hz, 1H), 5.13 (s, 2H), 4.59-4.54 (m, 2H), 4.54-4.47 (m, 3H), 3.86-3.81 (m, 2H), 3.68-3.63 (m, 2H), 3.62-3.51 (m, 12H), 3.45 (q, J=5.0 Hz, 2H), 2.02-1.89 (m, 1H), 1.87-1.74 (m, 1H), 1.36 (s, 8H), 0.91 (s, 9H). LC/MS [M+1]: 1004.5

### BF141

### (S)-2-(6-(3-(2-((2-(2-((5-(1-((2-cyanopyridin-3-yl)methyl)-2,2-dimethyl-3-oxo-2,3-dihydro-1H-pyrrolo[2,3-b]pyridin-6-yl)pyrimidin-2-yl)oxy)ethoxy)ethyl)amino)-2-oxoethoxy)phenoxy)nicotinamido)-5,5-dimethylhexanoic acid

¹H NMR (400 MHz, METHANOL-d4) δ=9.08 (s, 2H) 8.60 (td, J=4.46, 1.96 Hz, 2H) 8.21 (dd, J=8.68, 2.45 Hz, 1H) 7.98-8.04 (m, 2H) 7.60 (dd, J=8.19, 4.65 Hz, 1H) 7.26-7.36 (m, 2H) 6.96 (d, J=8.56 Hz, 1H) 6.83 (dd, J=8.31, 1.83 Hz, 1H) 6.78 (t, J=2.26 Hz, 1H) 6.73 (dd, J=7.89, 1.77 Hz, 1H) 5.13 (s, 2H) 4.56 (dd, J=5.44, 3.73 Hz, 2H) 4.45-4.54 (m, 3H) 3.80-3.86 (m, 2H) 3.61-3.67 (m, 2H) 3.47 (q, J=5.42 Hz, 2H) 1.90-2.00 (m, 1H) 1.74-1.87 (m, 1H) 0.91 (s, 9H) 1.27-1.42 (m, 8H). LC/MS [M+1]: 872.4

### BF142

### (S)-2-(6-(3-(2-((2-(2-(2-((5-(1-((2-cyanopyridin-3-yl)methyl)-2,2-dimethyl-3-oxo-2,3-dihydro-1H-pyrrolo[2,3-b]pyridin-6-yl)pyrimidin-2-yl)oxy)ethoxy)ethoxy)ethyl)amino)-2-oxoethoxy)phenoxy)nicotinamido)-5,5-dimethylhexanoic acid

¹H NMR (400 MHz, METHANOL-d4) δ=9.08 (s, 2H) 8.57-8.64 (m, 2H) 8.21 (dd, J=8.68, 2.45 Hz, 1H) 7.98-8.06 (m, 2H) 7.60 (dd, J=8.07, 4.77 Hz, 1H) 7.27-7.38 (m, 2H) 6.96 (d, J=8.68 Hz, 1H) 6.85 (dd, J=8.31, 2.08 Hz, 1H) 6.79 (t, J=2.20 Hz, 1H) 6.74 (dd, J=8.13, 1.77 Hz, 1H) 5.13 (s, 2H) 4.54-4.58 (m, 2H) 4.47-4.52 (m, 3H) 3.83-3.88 (m, 2H) 3.64-3.69 (m, 2H) 3.54-3.62 (m, 4H) 3.44 (q, J=5.46 Hz, 2H) 1.89-2.01 (m, 1H) 1.74-1.87 (m, 1H) 1.27-1.39 (m, 8H) 0.91 (s, 9H). LC/MS [M+1]: 916.4

### BF144

### (2S)-2-[[6-[3-[2-[2-[2-[2-[2-[[1-[5-[5-[1-[(2-cyano-3-pyridyl)methyl]-2,2-dimethyl-3-oxo- pyrrolo[2,3-b]pyridin-6-yl]pyrimidin-2-yl]oxypentyl]triazol-4-yl]methoxy]ethoxy]ethoxy]ethoxy]ethylamino]-2-oxo-ethoxy]phenoxy]pyridine-3-carbonyl]amino]-5,5-dimethyl-hexanoic acid

¹H NMR (400 MHz, METHANOL-d4) δ=9.10 (s, 2H) 8.62 (d, J=2.98 Hz, 2H) 8.24 (dd, J=8.64, 2.56 Hz, 1H) 8.02 (m, 2H) 7.96 (s, 1H) 7.61 (dd, J=8.11, 4.77 Hz, 1H) 7.36 (m, 2H) 6.99 (d, J=8.70 Hz, 1H) 6.87 (dd, J=8.23, 2.15 Hz, 1H) 6.81 (t, J=2.26 Hz, 1H) 6.78 (dd, J=8.11, 1.55 Hz, 1H) 5.13 (s, 2H) 4.59 (s, 2H) 4.51 (s, 2H) 4.49 (m, 1H) 4.43 (td, J=6.65, 2.44 Hz, 4H) 3.58 (m, 14H) 3.43 (m, 2H) 1.97 (m, 3H) 1.84 (m, 3H) 1.47 (m, 2H) 1.36 (m, 8H) 0.91 (s, 9H). LC/MS [M+1]: 1127.3

### BF 051

### (2S)-2-[[6-[3-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethylcarbamoyl]phenoxy]pyridine-3-carbonyl]amino]-5,5-dimethyl-hexanoic acid

¹H NMR (400 MHz, METHANOL-d4) δ ppm 8.94 (s, 2H), 8.57-8.65 (m, 2H), 8.23 (dd, J=8.63, 2.13 Hz, 1H), 8.00-8.06 (m, 1H), 7.95 (d, J=8.00 Hz, 1H), 7.72 (d, J=7.75 Hz, 1H), 7.58-7.65 (m, 2H), 7.50 (br t, J=7.69 Hz, 1H), 7.30 (d, J=8.00 Hz, 2H), 7.03 (br d, J=8.50 Hz, 1H), 5.12 (s, 2H), 4.60-4.80 (m, 2H), 4.46-4.57 (m, 2H), 4.16-4.34 (m, 2H), 3.63-3.74 (m, 6H), 3.54-3.62 (m, 2H), 3.36 (br d, J=2.13 Hz, 2H), 3.21-3.30 (m, 2H), 1.89-2.01 (m, 1H), 1.74-1.86 (m, 1H), 1.30-1.41 (m, 8H), 1.06-1.22 (m, 3H), 0.91 (s, 9H). LC/MS [M+1]: 982.5

### BF056

### (2S)-2-[[6-[3-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethylcarbamoyl]phenoxy]pyridine-3-carbonyl]amino]-5,5-dimethyl-hexanoic acid

1H NMR (400 MHz, METHANOL-d4) δ ppm 8.94 (s, 2H), 8.60 (td, J=4.22, 1.81 Hz, 2H), 8.25 (dd, J=8.69, 2.44 Hz, 1H), 8.02 (d, J=7.25 Hz, 1H), 7.94 (d, J=8.00 Hz, 1H), 7.70 (br d, J=7.75 Hz, 1H), 7.57-7.64 (m, 2H), 7.50 (t, J=7.94 Hz, 1H), 7.26-7.34 (m, 2H), 7.05 (d, J=8.63 Hz, 1H), 5.11 (s, 2H), 4.54-4.73 (m, 2H), 4.50 (dd, J=8.82, 5.19 Hz, 1H), 4.12-4.41 (m, 3H), 3.81-3.91 (m, 0.5H), 3.62-3.68 (m, 10H), 3.54-3.58 (m, 2H), 3.34-3.49 (m, 2H), 3.21-3.28 (m, 0.5H), 2.96-3.10 (m, 1H), 1.89-2.02 (m, 1H), 1.74-1.87 (m, 1H), 1.32-1.40 (m, 8H), 1.11-1.23 (m, 3H), 0.91 (s, 9H). LC/MS [M+1]: 1026.4

### BF031

### (2S)-2-[[6-[3-[2-[2-[2-[2-[2-[(2R)-4-[5-[1-[(2-cyano-3-pyridyl)methyl]-2,2-dimethyl-3-oxo- pyrrolo[2,3-b]pyridin-6-yl]pyrimidin-2-yl]-2-methyl-piperazin-1-yl]-2-oxo-ethoxy]ethoxy]ethoxy]ethoxy]ethylcarbamoyl]phenoxy]pyridine-3-carbonyl]amino]-5,5-dimethyl-hexanoic acid

1H NMR (400 MHz, METHANOL-d4) δ=8.95 (s, 2H), 8.63-8.59 (m, 2H), 8.26 (dd, J=2.4, 8.7 Hz, 1H), 8.02 (dd, J=1.3, 8.1 Hz, 1H), 7.95 (d, J=8.0 Hz, 1H), 7.71 (d, J=7.6 Hz, 1H), 7.63-7.61 (m, 1H), 7.61-7.58 (m, 1H), 7.51 (t, J=7.9 Hz, 1H), 7.34-7.28 (m, 2H), 7.06 (d, J=8.6 Hz, 1H), 5.11 (s, 2H), 4.74-4.55 (m, 2H), 4.51 (dd, J=5.1, 8.9 Hz, 1H), 4.40-4.13 (m, 3H), 3.91-3.82 (m, 0.5H), 3.67-3.58 (m, 14H), 3.57-3.53 (m, 2H), 3.47-3.36 (m, 2.5H), 3.12-2.98 (m, 1H), 2.02-1.90 (m, 1H), 1.88-1.75 (m, 1H), 1.38-1.31 (m, 8H), 1.25-1.13 (m, 3H), 0.91 (s, 9H). LC/MS [M+1]: 1070.6

### BF043

### (2S)-2-[[6-[3-[[10-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-10-oxo-decyl]carbamoyl]phenoxy]pyridine-3-carbonyl]amino]-5,5-dimethyl-hexanoic acid LC/MS [M+1]: 1063.5

¹H NMR (400 MHz, METHANOL-d4) δ ppm 8.95 (s, 2H), 8.59-8.64 (m, 2H), 8.50 (br t, J=6.02 Hz, 1H), 8.27 (dd, J=8.70, 2.50 Hz, 1H), 8.03 (dd, J=8.17, 1.25 Hz, 1H), 7.94 (d, J=7.99 Hz, 1H), 7.71 (d, J=7.87 Hz, 1H), 7.59-7.64 (m, 2H), 7.53 (t, J=7.93 Hz, 1H), 7.28-7.35 (m, 2H), 7.07 (d, J=8.70 Hz, 1H), 5.12 (s, 2H), 4.61-4.78 (m, 3H), 4.51 (dd, J=8.82, 5.13 Hz, 1H), 4.21-4.42 (m, 1H), 3.97-4.20 (m, 2H), 3.74-3.84 (m, 0.5H) 3.42-3.51 (m, 0.5H), 3.34-3.39 (m, 2H), 3.15-3.28 (m, 1H), 3.01-3.15 (m, 1H), 2.27 (t, J=7.51 Hz, 2H), 1.91-2.01 (m, 1H), 1.75-1.87 (m, 1H), 1.55-1.68 (m, 4H), 1.33-1.40 (m, 16H), 1.21-1.33 (m, 3H), 1.10-1.20 (m, 2H), 0.92 (s, 9H).

### BF042

### (2S)-2-[[6-[3-[[12-[[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethyl]amino]-12-oxo-dodecyl]carbamoyl]phenoxy]pyridine-3-carbonyl]amino]-5,5-dimethyl-hexanoic acid

¹H NMR (400 MHz, METHANOL-d4) δ ppm 8.95 (s, 2H), 8.60-8.64 (m, 2H), 8.50 (br t, J=5.66 Hz, 1H), 8.27 (dd, J=8.70, 2.50 Hz, 1H), 8.00-8.06 (m, 1H), 7.94 (d, J=7.99 Hz, 1H), 7.71 (d, J=7.75 Hz, 1H), 7.58-7.64 (m, 2H), 7.53 (t, J=7.93 Hz, 1H), 7.28-7.36 (m, 2H), 7.07 (d, J=8.58 Hz, 1H), 5.12 (s, 2H), 4.58-4.78 (m, 3H), 4.51 (dd, J=8.94, 5.13 Hz, 1H), 4.21-4.42 (m, 1H), 3.96-4.21 (m, 2H), 3.69-3.85 (m, 0.5H), 3.48 (br dd, J=3.16, 1.61 Hz, 0.5H), 3.34-3.37 (m, 2H), 3.17-3.28 (m, 1H), 3.00-3.14 (m, 1H), 2.27 (t, J=7.57 Hz, 2H), 1.90-2.02 (m, 1H), 1.75-1.88 (m, 1H), 1.58-1.65 (m, 4H), 1.31 (br s, 20H), 1.22-1.30 (m, 3H), 1.10-1.19 (m, 2H), 0.92 (s, 9H). LC/MS [M+1]: 1091.5

### BF050

### (2S)-2-[[2-[3-[[5-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethylcarbamoyl]-2-pyridyl]oxy]phenoxy]acetyl]amino]-5,5-dimethyl-hexanoic acid

¹H NMR (400 MHz, DMSO-d₆) δ=8.99-8.90 (m, 2H), 8.66 (br s, 2H), 8.43 (br s, 1H), 8.27 (br d, J=8.1 Hz, 1H), 7.99 (br s, 1H), 7.96-7.90 (m, 1H), 7.88-7.76 (m, 1H), 7.72-7.61 (m, 1H), 7.33 (q, J=8.4 Hz, 2H), 7.04 (br d, J=8.2 Hz, 1H), 6.88-6.72 (m, 3H), 5.12-5.01 (m, 2H), 4.64-4.43 (m, 5H), 4.42-4.35 (m, 1H), 4.18 (br s, 2H), 3.64-3.53 (m, 6H), 3.52-3.48 (m, 2H), 3.33-3.25 (m, 2H), 3.24-3.01 (m, 2H), 1.88-1.68 (m, 2H), 1.38-1.25 (m, 8H), 1.10 (br d, J=6.1 Hz, 3H), 0.95-0.82 (m, 9H). LC/MS [M+1]: 1012.4

### BF055

### (2S)-2-[[2-[3-[[5-[2-[2-[2-[2-[(2R)-4-[5-[1-[(2-cyano-3-pyridyl)methyl]-2,2-dimethyl-3-oxo- pyrrolo[2,3-b]pyridin-6-yl]pyrimidin-2-yl]-2-methyl-piperazin-1-yl]-2-oxo-ethoxy]ethoxy]ethoxy]ethylcarbamoyl]-2-pyridyl]oxy]phenoxy]acetyl]amino]-5,5-dimethyl-hexanoic acid

¹H NMR (400 MHz, METHANOL-d4) δ ppm 8.96 (s, 2H) 8.58-8.64 (m, 2H) 8.25 (dd, J=8.75, 2.50 Hz, 1H) 8.02 (dd, J=8.07, 1.31 Hz, 1H) 7.95 (d, J=8.00 Hz, 1H) 7.60 (dd, J=8.13, 4.75 Hz, 1H) 7.35 (t, J=8.13 Hz, 1H) 7.30 (d, J=8.00 Hz, 1H) 7.00 (d, J=8.75 Hz, 1H) 6.87 (br d, J=8.38 Hz, 1H) 6.76-6.82 (m, 2H) 5.11 (s, 2H) 4.55-4.74 (m, 2H) 4.48-4.53 (m, 3H) 4.08-4.40 (m, 3H) 3.89 (br d, J=14.63 Hz, 1H) 3.65 (s, 3H) 3.55-3.63 (m, 6H) 3.44-3.48 (m, 2H) 3.32-3.43 (m, 2H) 3.22-3.30 (m, 2H) 3.00-3.11 (m, 1H) 1.90-2.02 (m, 1H) 1.75-1.86 (m, 1H) 1.28-1.39 (m, 8H) 1.09-1.26 (m, 3H) 0.91 (s, 9H). LC/MS [M+1]: 1056.6

### BF032

### (2S)-2-[[2-[3-[[5-[2-[2-[2-[2-[2-[(2R)-4-[5-[1-[(2-cyano-3-pyridyl)methyl]-2,2-dimethyl-3-oxo-pyrrolo[2,3-b]pyridin-6-yl]pyrimidin-2-yl]-2-methyl-piperazin-1-yl]-2-oxo-ethoxy]ethoxy]ethoxy]ethoxy]ethylcarbamoyl]-2-pyridyl]oxy]phenoxy]acetyl]amino]-5,5-dimethyl-hexanoic acid

¹H NMR (400 MHz, DMSO-d6) δ=8.94 (s, 2H), 8.66 (br s, 2H), 8.49-8.38 (m, 1H), 8.32-8.24 (m, 1H), 7.99 (br d, J=7.5 Hz, 1H), 7.95-7.88 (m, 1H), 7.79 (br d, J=1.7 Hz, 1H), 7.71-7.61 (m, 1H), 7.40-7.27 (m, 2H), 7.05 (br d, J=8.1 Hz, 1H), 6.92-6.72 (m, 3H), 5.06 (br s, 2H), 4.64-4.41 (m, 5H), 4.41-4.33 (m, 1H), 4.18 (br s, 2H), 3.58 (br s, 4H), 3.53 (br d, J=7.2 Hz, 8H), 3.49-3.45 (m, 4H), 3.32 (br d, J=5.5 Hz, 4H), 1.88-1.69 (m, 2H), 1.37-1.24 (m, 8H), 1.11 (br d, J=6.2 Hz, 3H), 0.88 (s, 9H). LC/MS [M+1]: 1100.6

### BF049

### (2S)-2-[[6-[3-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethylcarbamoyl]phenoxy]pyridine-3-carbonyl]amino]-5,5-dimethyl-hexanoic acid

1H NMR (400 MHz, METHANOL-d4) δ=8.96 (br s, 2H), 8.60 (dd, J=1.5, 4.7 Hz, 1H), 8.57 (d, J=2.0 Hz, 1H), 8.23 (dd, J=2.4, 8.7 Hz, 1H), 8.00 (dd, J=1.5, 8.2 Hz, 1H), 7.97 (d, J=7.9 Hz, 1H), 7.70-7.65 (m, 1H), 7.62-7.56 (m, 2H), 7.49 (t, J=7.9 Hz, 1H), 7.31-7.26 (m, 2H), 7.05-7.00 (m, 1H), 5.11 (s, 2H), 4.50 (dd, J=5.1, 8.9 Hz, 1H), 3.73-3.58 (m, 10H), 3.56-3.49 (m, 2H), 2.01-1.87 (m, 1H), 1.86-1.75 (m, 1H), 1.39-1.27 (m, 8H), 0.91 (s, 9H). LC/MS [M+1]: 885.4

### BF030

### (2S)-2-[[6-[3-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethoxy]ethylcarbamoyl]phenoxy]pyridine-3-carbonyl]amino]-5,5-dimethyl-hexanoic acid

1H NMR (400 MHz, METHANOL-d4) δ=8.97 (s, 2H), 8.64-8.55 (m, 2H), 8.24 (dd, J=2.5, 8.6 Hz, 1H), 8.04-7.94 (m, 2H), 7.71-7.66 (m, 1H), 7.62-7.57 (m, 2H), 7.49 (t, J=7.9 Hz, 1H), 7.32-7.26 (m, 2H), 7.03 (d, J=8.6 Hz, 1H), 5.11 (s, 2H), 4.50 (dd, J=5.1, 8.8 Hz, 1H), 3.73-3.58 (m, 14H), 3.58-3.51 (m, 2H), 2.01-1.90 (m, 1H), 1.87-1.75 (m, 1H), 1.41-1.28 (m, 8H), 0.91 (s, 9H). LC/MS [M+1]: 929.6

### BF060

### (2S)-2-[[6-[3-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethylcarbamoyl]phenoxy]pyridine-3-carbonyl]amino]-5,5-dimethyl-hexanoic acid

1H NMR (400 MHz, METHANOL-d4) δ ppm 8.94 (s, 2H), 8.54-8.63 (m, 2H), 8.25 (dd, J=8.64, 2.44 Hz, 1H), 7.95-8.04 (m, 2H), 7.57-7.63 (m, 2H), 7.54 (t, J=1.91 Hz, 1H), 7.42 (t, J=7.93 Hz, 1H), 7.22-7.31 (m, 2H), 7.04 (d, J=8.70 Hz, 1H), 5.11 (s, 2H), 4.51 (dd, J=8.82, 5.13 Hz, 1H), 3.64-3.78 (m, 6H), 3.58 (d, J=5.13 Hz, 2H), 1.89-2.07 (m, 1H), 1.74-1.88 (m, 1H), 1.29-1.44 (m, 8H), 0.92 (s, 9H). LC/MS [M+1]: 841.4

### BF067

### (2S)-2-[[6-[3-[2-[2-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethoxy]ethoxy]ethoxy]ethylcarbamoyl]phenoxy]pyridine-3-carbonyl]amino]-5,5-dimethyl-hexanoic acid

¹H NMR (400 MHz, METHANOL-d4) δ ppm 8.98-9.08 (m, 2H), 8.57-8.67 (m, 2H), 8.22-8.31 (m, 1H), 7.99 (dd, J=7.38, 3.50 Hz, 2H), 7.69 (br d, J=7.88 Hz, 1H), 7.57-7.65 (m, 2H), 7.50 (t, J=7.94 Hz, 1H), 7.28-7.38 (m, 2H), 7.06 (d, J=8.63 Hz, 1H), 5.11 (s, 2H), 4.51 (dd, J=8.82, 5.19 Hz, 1H), 3.68-3.73 (m, 4H), 3.52-3.64 (m, 20H), 1.90-2.01 (m, 1H), 1.76-1.88 (m, 1H), 1.31-1.39 (m, 8H), 0.89-0.94 (m, 9H). LC/MS [M+1]: 1017.5

### BF062

### (2S)-2-[[6-[3-[2-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethoxy]ethoxy]ethylcarbamoyl]phenoxy]pyridine-3-carbonyl]amino]-5,5-dimethyl-hexanoic acid

1H NMR (400 MHz, METHANOL-d4) δ=9.07 (s, 2H), 8.63-8.57 (m, 2H), 8.26 (dd, J=2.5, 8.6 Hz, 1H), 8.02-7.98 (m, 2H), 7.68 (d, J=7.9 Hz, 1H), 7.63-7.57 (m, 2H), 7.50 (t, J=7.9 Hz, 1H), 7.38-7.28 (m, 2H), 7.06 (d, J=8.6 Hz, 1H), 5.12 (s, 2H), 4.51 (dd, J=5.2, 8.8 Hz, 1H), 3.71 (s, 4H), 3.65-3.47 (m, 16H), 2.03-1.90 (m, 1H), 1.89-1.73 (m, 1H), 1.36 (s, 8H), 0.92 (s, 9H). LC/MS [M+1]: 973.4

### BF063

### (2S)-2-[[2-[3-[[5-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethoxy]ethylcarbamoyl]-2-pyridyl]oxy]phenoxy]acetyl]amino]-5,5-dimethyl-hexanoic acid

¹H NMR (400 MHz, METHANOL-d4) δ=9.09 (s, 2H), 8.66-8.56 (m, 2H), 8.24 (dd, J=2.5, 8.6 Hz, 1H), 8.05-7.96 (m, 2H), 7.64-7.57 (m, 1H), 7.38-7.29 (m, 2H), 6.99 (d, J=8.5 Hz, 1H), 6.84 (s, 1H), 6.80-6.71 (m, 2H), 5.11 (s, 2H), 4.56-4.37 (m, 3H), 3.72 (s, 4H), 3.63 (br d, J=2.3 Hz, 4H), 3.60-3.49 (m, 6H), 3.47-3.35 (m, 2H), 2.03-1.90 (m, 1H), 1.89-1.71 (m, 1H), 1.40-1.25 (m, 8H), 0.98-0.85 (m, 9H). LC/MS [M+1]: 959.4

### BF068

### (2S)-2-[[2-[3-[[5-[2-[2-[2-[2-[2-[2-[[5-[1-[(2-cyano-3-pyridyl)methyl]-2,2-dimethyl-3-oxo- pyrrolo[2,3-b]pyridin-6-yl]pyrimidin-2-yl]amino]ethoxy]ethoxy]ethoxy]ethoxy]ethoxy]ethylcarbamoyl]-2-pyridyl]oxy]phenoxy]acetyl]amino]-5,5-dimethyl-hexanoic acid

¹H NMR (400 MHz, METHANOL-d4) δ ppm 9.08 (s, 2H), 8.60-8.65 (m, 2H), 8.25 (dd, J=8.63, 2.50 Hz, 1H), 7.97-8.05 (m, 2H), 7.61 (dd, J=8.13, 4.63 Hz, 1H), 7.31-7.40 (m, 2H), 7.01 (d, J=8.63 Hz, 1H), 6.83-6.89 (m, 1H), 6.73-6.81 (m, 2H), 5.11 (s, 2H), 4.48-4.55 (m, 3H), 3.73 (s, 4H), 3.63-3.66 (m, 2H), 3.52-3.61 (m, 16H), 3.40-3.45 (m, 2H), 1.91-2.01 (m, 1H), 1.76-1.87 (m, 1H), 1.31-1.40 (m, 8H), 0.92 (s, 9H). LC/MS [M+1]: 1047.8

### BF057

### (2S)-2-[[2-[3-[[5-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethylcarbamoyl]-2-pyridyl]oxy]phenoxy]acetyl]amino]-5,5-dimethyl-hexanoic acid

¹H NMR (400 MHz, METHANOL-d4) δ=8.98 (br s, 2H), 8.62-8.56 (m, 2H), 8.21 (dd, J=2.6, 8.7 Hz, 1H), 8.01-7.94 (m, 2H), 7.59 (dd, J=4.6, 8.1 Hz, 1H), 7.35-7.27 (m, 2H), 6.96 (d, J=8.7 Hz, 1H), 6.86-6.80 (m, 1H), 6.78-6.71 (m, 2H), 5.10 (s, 2H), 4.55-4.47 (m, 3H), 3.68 (s, 4H), 3.64-3.55 (m, 6H), 3.49-3.43 (m, 2H), 2.02-1.91 (m, 1H), 1.88-1.75 (m, 1H), 1.36 (s, 8H), 0.92 (s, 9H). LC/MS [M+1]: 915.3

### BF071

### (2S)-2-[[2-[3-[[5-[2-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethoxy]ethoxy]ethoxy]ethylcarbamoyl]-2-pyridyl]oxy]phenoxy]acetyl]amino]-5,5-dimethyl-hexanoic acid

¹H NMR (400 MHz, METHANOL-d4) δ=8.87 (s, 2H), 8.66-8.58 (m, 2H), 8.24 (dd, J=2.4, 8.6 Hz, 1H), 8.01 (dd, J=1.3, 8.2 Hz, 1H), 7.93 (d, J=8.0 Hz, 1H), 7.60 (dd, J=4.8, 8.1 Hz, 1H), 7.34 (t, J=8.2 Hz, 1H), 7.26 (d, J=8.1 Hz, 1H), 6.99 (d, J=8.6 Hz, 1H), 6.91-6.86 (m, 1H), 6.83-6.74 (m, 2H), 5.11 (s, 2H), 4.52 (s, 3H), 3.66-3.53 (m, 18H), 3.45 (d, J=5.4 Hz, 2H), 2.00-1.93 (m, 1H), 1.84-1.78 (m, 1H), 1.38-1.30 (m, 8H), 0.91 (s, 9H). LC/MS [M+1]: 1003.8

### BF072

### (2S)-2-[[2-[3-[[5-[2-[2-[[5-[1-[(2-cyano-3-pyridyl)methyl]-2,2-dimethyl-3-oxo-pyrrolo[2,3-b]pyridin-6-yl]pyrimidin-2-yl]amino]ethoxy]ethylcarbamoyl]-2-pyridyl]oxy]phenoxy]acetyl]amino]-5,5-dimethyl-hexanoic acid LC/MS [M+1]: 871.4

¹H NMR (400 MHz, METHANOL-d4) δ=9.02 (s, 2H), 8.66-8.56 (m, 2H), 8.24 (dd, J=2.4, 8.6 Hz, 1H), 8.04-7.94 (m, 2H), 7.60 (dd, J=4.8, 8.1 Hz, 1H), 7.37-7.24 (m, 2H), 6.99 (d, J=8.7 Hz, 1H), 6.89-6.81 (m, 1H), 6.79-6.71 (m, 2H), 5.11 (s, 2H), 4.57-4.45 (m, 3H), 3.68 (s, 4H), 3.63-3.57 (m, 2H), 3.52-3.44 (m, 2H), 2.01-1.92 (m, 1H), 1.88-1.74 (m, 1H), 1.42-1.29 (m, 8H), 0.92 (s, 9H).

### BF059

### (2S)-2-[[6-[3-[2-[2-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]oxyethoxy]ethoxy]ethoxy]ethoxy]ethoxy]ethylcarbamoyl]phenoxy]pyridine-3-carbonyl]amino]-5,5-dimethyl-hexanoic acid

1H NMR (400 MHz, METHANOL-d4) δ=9.10 (s, 2H), 8.71-8.53 (m, 2H), 8.25 (dd, J=2.4, 8.7 Hz, 1H), 8.11-7.96 (m, 2H), 7.71 (d, J=7.9 Hz, 1H), 7.64-7.57 (m, 2H), 7.51 (t, J=7.9 Hz, 1H), 7.38 (d, J=7.9 Hz, 1H), 7.31 (dd, J=2.0, 7.9 Hz, 1H), 7.06 (d, J=8.6 Hz, 1H), 5.13 (s, 2H), 4.61-4.55 (m, 2H), 4.50 (dd, J=5.2, 8.8 Hz, 1H), 3.88-3.82 (m, 2H), 3.72-3.49 (m, 20H), 2.03-1.89 (m, 1H), 1.87-1.73 (m, 1H), 1.36 (s, 6H), 1.34-1.24 (m, 2H), 0.91 (s, 9H). LC/MS [M+1]: 1018.6

### BF053

### (2S)-2-[[6-[3-[2-[2-[[5-[1-[(2-cyano-3-pyridyl)methyl]-2,2-dimethyl-3-oxo-pyrrolo[2,3-b]pyridin-6-yl]pyrimidin-2-yl]oxyethoxy]ethylcarbamoyl]phenoxy]pyridine-3-carbonyl]amino]-5,5-dimethyl-hexanoic acid

¹H NMR (400 MHz, METHANOL-d4) δ=9.07 (s, 2H), 8.63-8.58 (m, 2H), 8.24 (dd, J=2.5, 8.6 Hz, 1H), 8.05-7.99 (m, 2H), 7.69-7.65 (m, 1H), 7.63-7.59 (m, 1H), 7.59 (dd, J=1.7, 3.9 Hz, 1H), 7.46 (t, J=7.9 Hz, 1H), 7.37 (d, J=7.9 Hz, 1H), 7.29-7.25 (m, 1H), 7.03 (d, J=8.6 Hz, 1H), 5.14 (s, 2H), 4.50 (br dd, J=3.0, 8.4 Hz, 1H), 3.91-3.86 (m, 2H), 3.75-3.69 (m, 2H), 3.59-3.53 (m, 2H), 3.26-3.20 (m, 2H), 2.00-1.87 (m, 1H), 1.85-1.73 (m, 1H), 1.38-1.30 (m, 8H), 0.90 (s, 9H). LC/MS [M+1]: 842.3

### BF054

### (2S)-2-[[6-[3-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]oxyethoxy]ethoxy]ethoxy]ethylcarbamoyl]phenoxy]pyridine-3-carbonyl]amino]-5,5-dimethyl-hexanoic acid

¹H NMR (400 MHz, METHANOL-d4) δ ppm 9.09 (s, 2H), 8.57-8.63 (m, 2H), 8.21-8.27 (m, 1H), 7.98-8.06 (m, 2H), 7.70 (d, J=7.75 Hz, 1H), 7.57-7.64 (m, 2H), 7.49 (t, J=7.94 Hz, 1H), 7.37 (d, J=8.00 Hz, 1H), 7.29 (dd, J=7.69, 2.06 Hz, 1H), 7.04 (d, J=8.63 Hz, 1H), 5.13 (s, 2H), 4.53-4.56 (m, 2H), 4.49 (br dd, J=8.44, 5.07 Hz, 1H), 3.80-3.84 (m, 2H), 3.61-3.65 (m, 10H), 3.52-3.56 (m, 2H), 1.90-2.01 (m, 1H), 1.74-1.86 (m, 1H), 1.36 (s, 6H), 1.30-1.35 (m, 2H), 0.90 (s, 9H). LC/MS [M+1]: 930.5

### BF052

### (2S)-2-[[6-[3-[2-[2-[2-[2-[2-[[5-[1-[(2-cyano-3-pyridyl)methyl]-2,2-dimethyl-3-oxo-pyrrolo[2,3-b]pyridin-6-yl]pyrimidin-2-yl]oxyethoxy]ethoxy]ethoxy]ethoxy]ethylcarbamoyl]phenoxy]pyridine-3-carbonyl]amino]-5,5-dimethyl-hexanoic acid

1H NMR (400 MHz, METHANOL-d4) δ=9.10 (s, 2H), 8.63-8.57 (m, 2H), 8.25 (dd, J=2.4, 8.6 Hz, 1H), 8.01 (dd, J=7.9, 11.8 Hz, 2H), 7.71 (br d, J=7.8 Hz, 1H), 7.63-7.58 (m, 2H), 7.50 (t, J=7.9 Hz, 1H), 7.36 (d, J=8.0 Hz, 1H), 7.30 (dd, J=1.5, 8.0 Hz, 1H), 7.05 (d, J=8.5 Hz, 1H), 5.13 (s, 2H), 4.50 (dd, J=5.3, 8.8 Hz, 1H), 3.87-3.82 (m, 2H), 3.67-3.53 (m, 18H), 2.00-1.91 (m, 1H), 1.86-1.76 (m, 1H), 1.36 (s, 8H), 0.91 (s, 9H).

LC/MS [M+1]: 974.3

### BF070

### (2S)-2-[[6-[3-[2-[2-[2-[5-[1-[(2-cyano-3-pyridyl)methyl]-2,2-dimethyl-3-oxo-pyrrolo[2,3-b]pyridin-6-yl]pyrimidin-2-yl]oxyethoxy]ethoxy]ethylcarbamoyl]phenoxy]pyridine-3-carbonyl]amino]-5,5-dimethyl-hexanoic acid

1H NMR (400 MHz, METHANOL-d4) δ=9.08 (s, 2H), 8.64-8.55 (m, 2H), 8.22 (dd, J=2.5, 8.6 Hz, 1H), 8.06-7.97 (m, 2H), 7.69 (d, J=8.0 Hz, 1H), 7.63-7.57 (m, 2H), 7.48 (t, J=7.9 Hz, 1H), 7.37 (d, J=8.0 Hz, 1H), 7.31-7.25 (m, 1H), 7.02 (d, J=8.6 Hz, 1H), 5.13 (s, 2H), 4.55 (dd, J=3.9, 5.4 Hz, 2H), 4.49 (br d, J=3.6 Hz, 1H), 3.86 (dd, J=3.8, 5.4 Hz, 2H), 3.75-3.68 (m, 2H), 3.67-3.60 (m, 4H), 3.55 (d, J=5.3 Hz, 2H), 2.02-1.90 (m, 1H), 1.88-1.74 (m, 1H), 1.36 (s, 6H), 1.35-1.27 (m, 2H), 0.91 (s, 9H). LC/MS [M+1]: 886.5

### BF065

### (2S)-2-[[2-[3-[[5-[2-[2-[[5-[1-[(2-cyano-3-pyridyl)methyl]-2,2-dimethyl-3-oxo-pyrrolo[2,3-b]pyridin-6-yl]pyrimidin-2-yl]oxyethoxy]ethylcarbamoyl]-2-pyridyl]oxy]phenoxy]acetyl]amino]-5,5-dimethyl-hexanoic acid

1H NMR (400 MHz, METHANOL-d4) δ=9.07 (s, 2H), 8.60 (dt, J=1.8, 4.2 Hz, 2H), 8.21 (dd, J=2.5, 8.6 Hz, 1H), 8.02 (d, J=8.1 Hz, 1H), 7.99 (d, J=7.9 Hz, 1H), 7.60 (dd, J=4.8, 8.1 Hz, 1H), 7.35-7.26 (m, 2H), 6.96 (d, J=8.7 Hz, 1H), 6.82 (dd, J=2.0, 8.3 Hz, 1H), 6.78 (t, J=2.2 Hz, 1H), 6.72 (dd, J=1.6, 8.1 Hz, 1H), 5.12 (s, 2H), 4.58-4.53 (m, 2H), 4.52-4.47 (m, 3H), 3.82 (dd, J=3.7, 5.2 Hz, 2H), 3.66-3.61 (m, 2H), 3.49-3.44 (m, 2H), 2.00-1.90 (m, 1H), 1.86-1.76 (m, 1H), 1.40-1.30 (m, 8H), 0.91 (s, 9H). LC/MS [M+1]: 872.4

### BF061

### (2S)-2-[[2-[3-[[5-[2-[2-[2-[2-[5-[1-[(2-cyano-3-pyridyl)methyl]-2,2-dimethyl-3-oxo-pyrrolo[2,3-b]pyridin-6-yl]pyrimidin-2-yl]oxyethoxy]ethoxy]ethoxy]ethylcarbamoyl]-2-pyridyl]oxy]phenoxy]acetyl]amino]-5,5-dimethyl-hexanoic acid

¹H NMR (400 MHz, METHANOL-d4) δ ppm 9.09-9.13 (m, 2H), 8.61 (d, J=2.88 Hz, 2H), 8.24 (dd, J=8.69, 2.44 Hz, 1H), 7.95-8.06 (m, 2H), 7.60 (dd, J=8.13, 4.63 Hz, 1H), 7.28-7.41 (m, 2H), 6.93-7.03 (m, 1H), 6.72-6.90 (m, 3H), 5.13 (s, 2H), 4.55-4.62 (m, 2H), 4.42-4.55 (m, 3H), 3.81-3.89 (m, 2H), 3.53-3.67 (m, 10H), 3.40-3.47 (m, 2H), 1.89-2.01 (m, 1H), 1.75-1.88 (m, 1H), 1.30-1.39 (m, 8H), 0.91 (s, 9H). LC/MS [M+1]: 960.4

### BF069

### (2S)-2-[[2-[3-[[5-[2-[2-[2-[2-[2-[2-[5-[1-[(2-cyano-3-pyridyl)methyl]-2,2-dimethyl-3-oxo- pyrrolo[2,3-b]pyridin-6-yl]pyrimidin-2-yl]oxyethoxy]ethoxy]ethoxy]ethoxy]ethoxy]ethylcarbamoyl]-2-pyridyl]oxy]phenoxy]acetyl]amino]-5,5-dimethyl-hexanoic acid

1H NMR (400 MHz, METHANOL-d4) δ=9.12 (s, 2H), 8.62 (d, J=2.6 Hz, 2H), 8.25 (dd, J=2.2, 8.6 Hz, 1H), 8.04-7.99 (m, 2H), 7.61 (dd, J=4.8, 7.9 Hz, 1H), 7.42-7.32 (m, 2H), 7.00 (d, J=8.8 Hz, 1H), 6.88 (td, J=1.2, 8.5 Hz, 1H), 6.81 (s, 1H), 6.78 (d, J=8.8 Hz, 1H), 5.13 (br s, 2H), 4.60 (br d, J=4.3 Hz, 1H), 4.52 (s, 2H), 3.87-3.83 (m, 2H), 3.67 (br dd, J=3.4, 6.2 Hz, 2H), 3.63-3.54 (m, 18H), 3.44 (br t, J=5.4 Hz, 2H), 2.02-1.89 (m, 1H), 1.87-1.75 (m, 1H), 1.37-1.31 (m, 8H), 0.91 (s, 9H). LC/MS [M+1]: 1048.3

### BF073

### (2S)-2-[[2-[3-[[5-[2-[2-[2-[2-[2-[5-[1-[(2-cyano-3-pyridyl)methyl]-2,2-dimethyl-3-oxo-pyrrolo[2,3-b]pyridin-6-yl]pyrimidin-2-yl]oxyethoxy]ethoxy]ethoxy]ethoxy]ethylcarbamoyl]-2-pyridyl]oxy]phenoxy]acetyl]amino]-5,5-dimethyl-hexanoic acid

¹H NMR (400 MHz, METHANOL-d4) δ=9.12 (s, 2H), 8.66-8.60 (m, 2H), 8.25 (dd, J=2.4, 8.6 Hz, 1H), 8.06-8.01 (m, 2H), 7.62 (dd, J=4.7, 8.1 Hz, 1H), 7.42-7.31 (m, 2H), 7.01 (d, J=8.5 Hz, 1H), 6.89 (dd, J=2.6, 8.1 Hz, 1H), 6.84-6.75 (m, 2H), 5.15 (s, 2H), 4.54 (s, 2H), 4.52-4.49 (m, 1H), 3.88-3.84 (m, 2H), 3.69-3.67 (m, 2H), 3.65-3.54 (m, 14H), 3.47 (br d, J=5.1 Hz, 2H), 1.97 (br d, J=6.9 Hz, 1H), 1.76 (s, 1H), 1.41-1.33 (m, 8H), 0.93 (s, 9H). LC/MS [M+1]: 502.8

### BF075

### (2S)-2-[[2-[3-[[5-[2-[2-[2-[2-[[1-[5-[5-[1-[(2-cyano-3-pyridyl)methyl]-2,2-dimethyl-3-oxo- pyrrolo[2,3-b]pyridin-6-yl]pyrimidin-2-yl]oxypentyl]triazol-4-yl]methoxy]ethoxy]ethoxy]ethoxy]ethylcarbamoyl]-2-pyridyl]oxy]phenoxy]acetyl]amino]-5,5-dimethyl-hexanoic acid

¹H NMR (400 MHz, METHANOL-d4) δ ppm 9.12 (s, 2H) 8.64 (d, J=4.53 Hz, 1H) 8.60 (d, J=2.38 Hz, 1H) 8.22 (dd, J=8.58, 2.50 Hz, 1H) 8.01-8.07 (m, 2H) 7.98 (s, 1H) 7.63 (dd, J=8.11, 4.65 Hz, 1H) 7.33-7.43 (m, 2H) 6.99 (d, J=8.58 Hz, 1H) 6.91 (dd, J=8.23, 2.26 Hz, 1H) 6.84 (t, J=2.15 Hz, 1H) 6.79 (dd, J=7.81, 1.61 Hz, 1H) 5.15 (s, 2H) 4.60 (d, J=1.67 Hz, 5H) 4.42-4.46 (m, 4H) 3.59-3.68 (m, 14H) 3.54-3.58 (m, 2H) 1.97-2.06 (m, 2H) 1.82-1.94 (m, 3H) 1.67-1.80 (m, 1H) 1.45-1.54 (m, 2H) 1.38 (s, 6H) 1.20 (dd, J=9.48, 7.69 Hz, 2H) 0.87 (s, 9H) LC/MS [M+1]: 1127.5

### BF080

¹H NMR (400 MHz, METHANOL-d4) δ ppm 8.88-8.98 (m, 2H) 8.25 (br d, J=5.13 Hz, 1H) 8.12 (d, J=7.87 Hz, 1H) 8.00 (s, 1H) 7.86-7.96 (m, 3H) 7.77-7.81 (m, 1H) 7.58-7.70 (m, 3H) 7.15-7.24 (m, 2H) 7.04 (d, J=8.70 Hz, 2H) 7.00-7.02 (m, 1H) 6.41-6.49 (m, 1H) 4.65 (s, 2H) 4.57 (s, 2H) 4.45 (q, J=6.99 Hz, 4H) 3.62-3.69 (m, 4H) 3.56-3.61 (m, 2H) 3.47-3.50 (m, 2H) 2.72 (s, 3H) 2.47 (s, 3H) 2.00 (dt, J=14.51, 7.11 Hz, 2H) 1.82-1.93 (m, 2H) 1.73 (d, J=6.91 Hz, 3H) 1.45-1.55 (m, 2H). LCMS (ESI⁺): m/z 1163.3 (M+H)

### Example S3: Peptide Synthesis

All peptides were synthesized on Discovery-12 peptide synthesizer (Peptide Machines, San Diego, CA, USA), using the standard Fmoc peptide chemistry.

The H-Leu-2-Cl-Trt and D-biotin were sourced from ChemImpex International, Inc. Fmoc-Gly, Fmoc-Ser (tBu), Fmoc-Leu, Fmoc-Arg (Pbf), Fmoc-Gln (Trt), Fmoc-Phe were from Chempep, Inc.

The ESI Mass Spec was done on Agilent G1946, and HPLC was done on Beckman Gold 167 System.

### Peptide Synthesis Protocol:

The peptide were assembled stepwise starting with H-Leu-2-CI-Trt resin on 0.2 mmol scale, using six-fold excess of Fmoc amino acids. Fmoc protecting group was removed using 20% piperidine in DMF and free amine was coupled with amino acids/TBTU/NMM in DMF or D-biotin. Upon completion of the synthesis the peptide-resin was treated with TFA/TIS/H2O (93:2:5 by vol.) for 2-3 h at RT to cleave peptide from the resin. Cleaved peptide was treated with cold diethyl ether to precipitate peptide out, then precipitated peptide was washed with diethyl ether three times. Crude peptide was purified by HPLC (UV detector 215 nm, RP-18 column) using linear solvent gradient of 0.1% TFA in water and 0.1% TFA in acetonitrile from 0% to 40% in 60 min. Final peptide was characterized by Mass Spectroscopy and HPLC.

### NHS Peptides Synthesis:

After cleavage and purification of the acetylated Cys containing peptides, they were dissolved in ACN-H₂O (1:1) and equimolar amount of the maleimide-PEG4-NHS was added. The reaction was completed overnight and solution was lyophilized and purified by HPLC. The resulting product was stored at −20° C.

### List of Compounds

TABLE A

| ID. | Structure |
|---|---|
| BF001 | 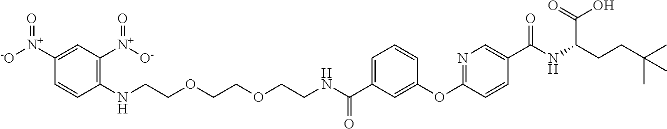 |
| BF002 | 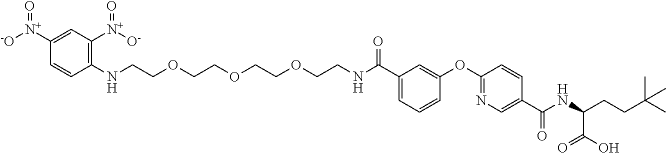 |
| BF003 | 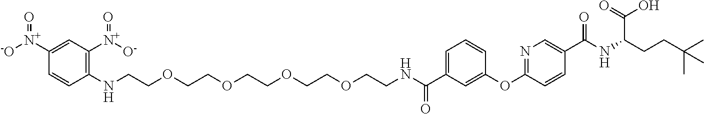 |
| BF004 | 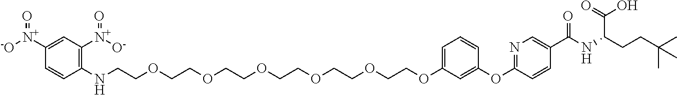 |
| BF005 | 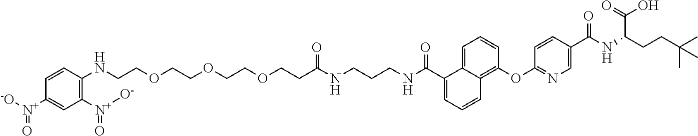 |
| BF006 | 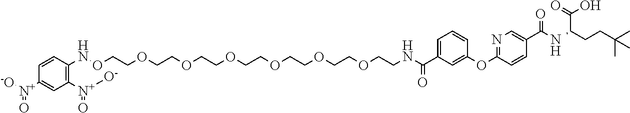 |
| BF007 | 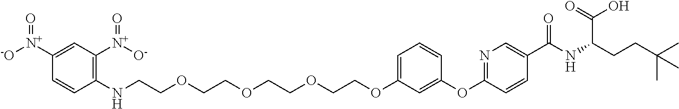 |
| BF008 | 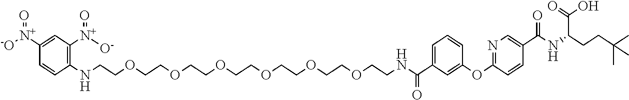 |
| BF009 | 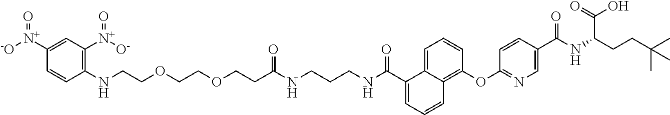 |
| BF010 | 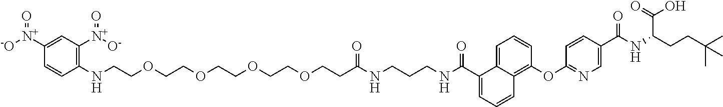 |
| BF011 | 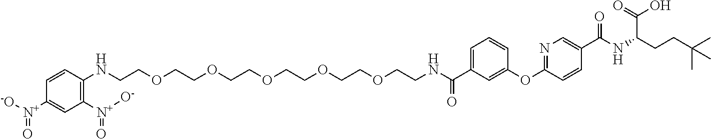 |
| BF012 | 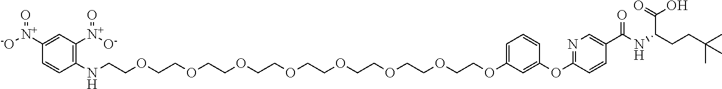 |
| BF013 | 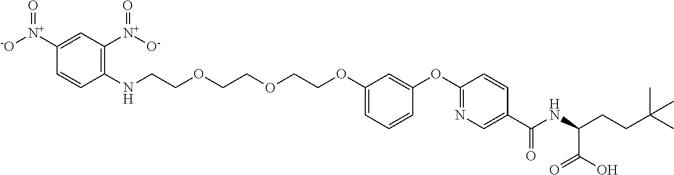 |
| BF014 | 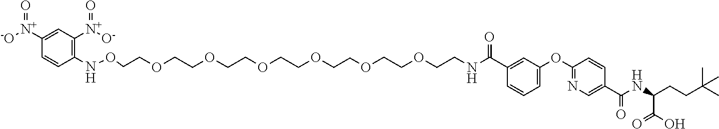 |
| BF015 | 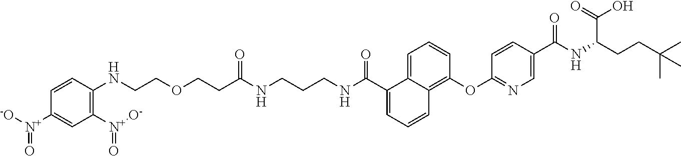 |
| BF016 | 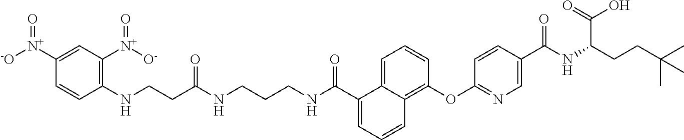 |
| BF017 | 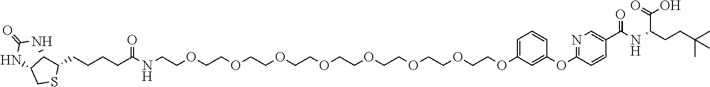 [SMILES: CC(C)(C)CC[C@H](NC(=O)c1ccc(Oc2cccc(OCCOCCOCCOCCOCCOCCOCCOCCNC(=O)CCCC[C@@H]3SC[C@@H]4NC(=O)N[C@@H]43)c2)nc1)C(=O)O] |
| BF018 | 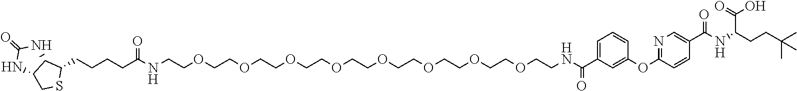 [SMILES: CC(C)(C)CC[C@H](NC(=O)c1ccc(Oc2cccc(C(=O)NCCOCCOCCOCCOCCOCCOCCOCCOCCNC(=O)CCCC[C@@H]3SC[C@@H]4NC(=O)N[C@@H]43)c2)nc1)C(=O)O] |
| BF019 | 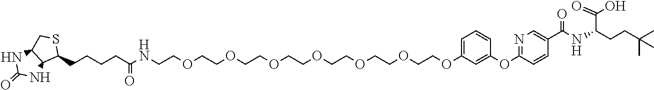 [SMILES: CC(C)(C)CC[C@H](NC(=O)c1ccc(Oc2cccc(OCCOCCOCCOCCOCCOCCOCCNC(=O)CCCC[C@@H]3SC[C@@H]4NC(=O)N[C@@H]43)c2)nc1)C(=O)O] |
| BF020 | 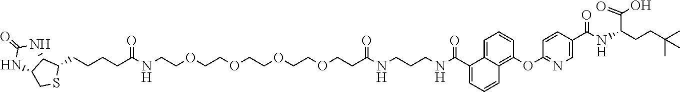 [SMILES: CC(C)(C)CC[C@H](NC(=O)c1ccc(Oc2cccc3c(C(=O)NCCCNC(=O)CCOCCOCCOCCOCCNC(=O)CCCC[C@@H]4SC[C@@H]5NC(=O)N[C@@H]54)cccc23)nc1)C(=O)O] |
| BF021 | 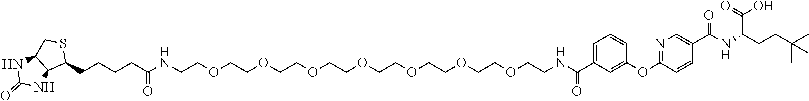 [SMILES: CC(C)(C)CC[C@H](NC(=O)c1ccc(Oc2cccc(C(=O)NCCOCCOCCOCCOCCOCCOCCOCCNC(=O)CCCC[C@@H]3SC[C@@H]4NC(=O)N[C@@H]43)c2)nc1)C(=O)O] |
| BF022 | 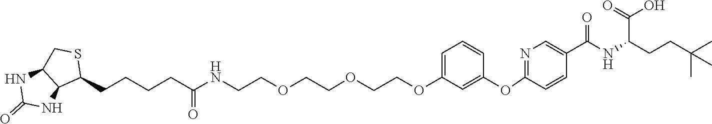 [SMILES: CC(C)(C)CC[C@H](NC(=O)c1ccc(Oc2cccc(OCCOCCOCCNC(=O)CCCC[C@@H]3SC[C@@H]4NC(=O)N[C@@H]43)c2)nc1)C(=O)O] |
| BF023 | 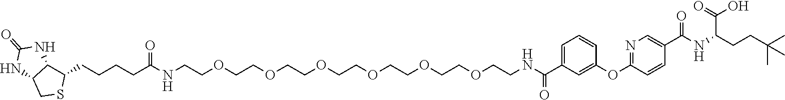 [SMILES: CC(C)(C)CC[C@H](NC(=O)c1ccc(Oc2cccc(C(=O)NCCOCCOCCOCCOCCOCCOCCNC(=O)CCCC[C@@H]3SC[C@@H]4NC(=O)N[C@@H]43)c2)nc1)C(=O)O] |
| BF024 | 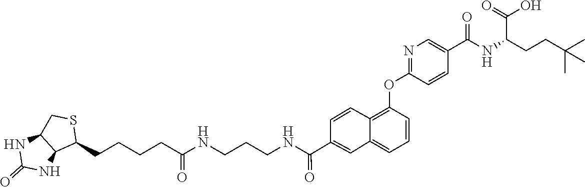 [SMILES: CC(C)(C)CC[C@H](NC(=O)c1ccc(Oc2cccc3cc(C(=O)NCCCNC(=O)CCCC[C@@H]4SC[C@@H]5NC(=O)N[C@@H]54)ccc23)nc1)C(=O)O] |
| BF025 | 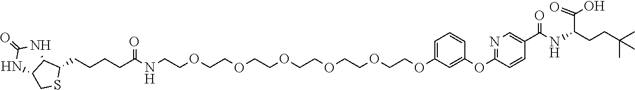 [SMILES: CC(C)(C)CC[C@H](NC(=O)c1ccc(Oc2cccc(OCCOCCOCCOCCOCCOCCNC(=O)CCCC[C@@H]3SC[C@@H]4NC(=O)N[C@@H]43)c2)nc1)C(=O)O] |
| BF026 | 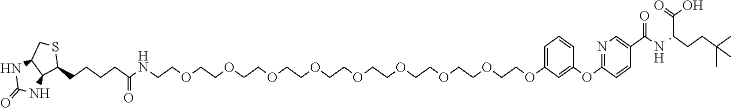 [SMILES: CC(C)(C)CC[C@H](NC(=O)c1ccc(Oc2cccc(OCCOCCOCCOCCOCCOCCOCCOCCOCCNC(=O)CCCC[C@@H]3SC[C@@H]4NC(=O)N[C@@H]43)c2)nc1)C(=O)O] |
| BF027 | 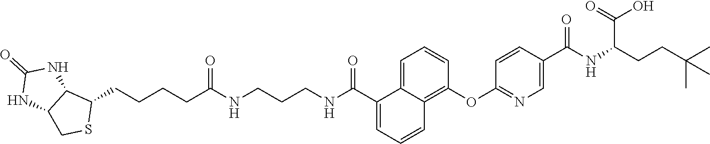 [SMILES: CC(C)(C)CC[C@H](NC(=O)c1ccc(Oc2cccc3c(C(=O)NCCCNC(=O)CCCC[C@@H]4SC[C@@H]5NC(=O)N[C@@H]54)cccc23)nc1)C(=O)O] |
| BF028 | 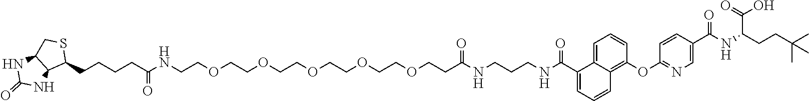 [SMILES: CC(C)(C)CC[C@H](NC(=O)c1ccc(Oc2cccc3c(C(=O)NCCCNC(=O)CCOCCOCCOCCOCCOCCNC(=O)CCCC[C@@H]4SC[C@@H]5NC(=O)N[C@@H]54)cccc23)nc1)C(=O)O] |
| BF029 | 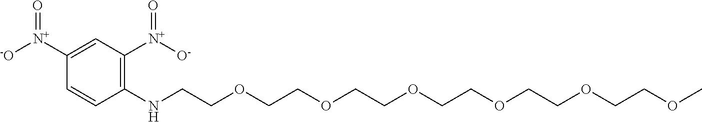 |
| BF030 | 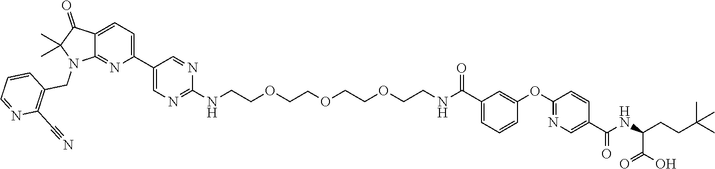 [SMILES: CC(C)(C)CC[C@H](NC(=O)c1ccc(Oc2cccc(C(=O)NCCOCCOCCOCCNc3ncc(-c4ccc5c(n4)N(Cc4cccnc4C#N)C(C)(C)C5=O)cn3)c2)nc1)C(=O)O] |
| BF031 | 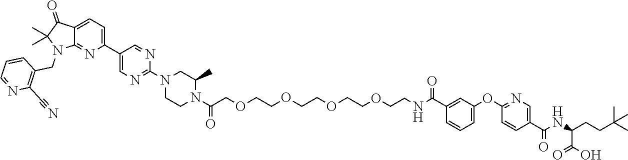 [SMILES: C[C@@H]1CN(c2ncc(-c3ccc4c(n3)N(Cc3cccnc3C#N)C(C)(C)C4=O)cn2)CCN1C(=O)COCCOCCOCCOCCNC(=O)c1cccc(Oc2ccc(C(=O)N[C@@H](CCC(C)(C)C)C(=O)O)cn2)c1] |
| BF032 | 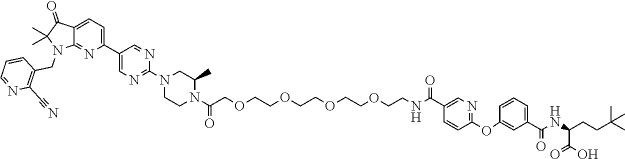 [SMILES: C[C@@H]1CN(c2ncc(-c3ccc4c(n3)N(Cc3cccnc3C#N)C(C)(C)C4=O)cn2)CCN1C(=O)COCCOCCOCCOCCNC(=O)c1ccc(Oc2cccc(C(=O)N[C@@H](CCC(C)(C)C)C(=O)O)c2)nc1] |
| BF033 | 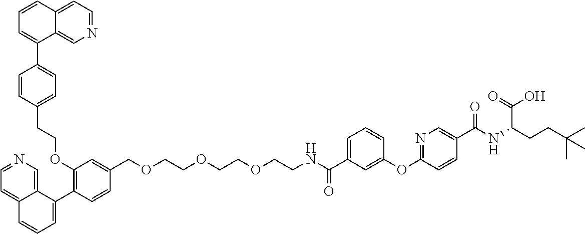 [SMILES: CC(C)(C)CC[C@H](NC(=O)c1ccc(Oc2cccc(C(=O)NCCOCCOCCOCc3ccc(-c4cccc5ccncc45)c(OCCc4ccc(-c5cccc6ccncc56)cc4)c3)c2)nc1)C(=O)O] |
| BF034 | 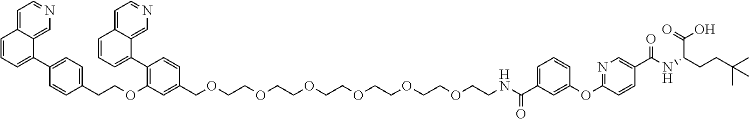 [SMILES: CC(C)(C)CC[C@H](NC(=O)c1ccc(Oc2cccc(C(=O)NCCOCCOCCOCCOCCOCCOCc3ccc(-c4cccc5ccncc45)c(OCCc4ccc(-c5cccc6ccncc56)cc4)c3)c2)nc1)C(=O)O] |
| BF035 | 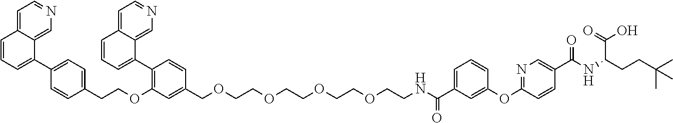 [SMILES: CC(C)(C)CC[C@H](NC(=O)c1ccc(Oc2cccc(C(=O)NCCOCCOCCOCCOCc3ccc(-c4cccc5ccncc45)c(OCCc4ccc(-c5cccc6ccncc56)cc4)c3)c2)nc1)C(=O)O] |
| BF036 | 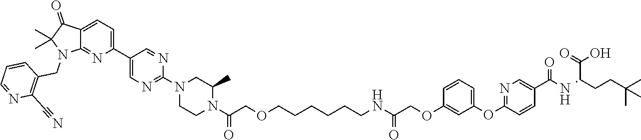 [SMILES: C[C@@H]1CN(c2ncc(-c3ccc4c(n3)N(Cc3cccnc3C#N)C(C)(C)C4=O)cn2)CCN1C(=O)COCCCCCCNC(=O)COc1cccc(Oc2ccc(C(=O)N[C@@H](CCC(C)(C)C)C(=O)O)cn2)c1] |
| BF037 | 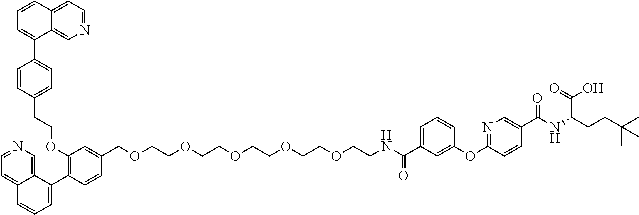 [SMILES: CC(C)(C)CC[C@H](NC(=O)c1ccc(Oc2cccc(C(=O)NCCOCCOCCOCCOCCOCc3ccc(-c4cccc5ccncc45)c(OCCc4ccc(-c5cccc6ccncc56)cc4)c3)c2)nc1)C(=O)O] |
| BF038 | 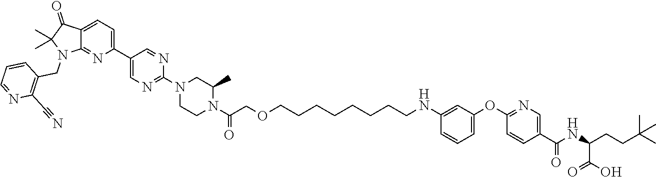 [SMILES: C[C@@H]1CN(c2ncc(-c3ccc4c(n3)N(Cc3cccnc3C#N)C(C)(C)C4=O)cn2)CCN1C(=O)COCCCCCCCCNc1cccc(Oc2ccc(C(=O)N[C@@H](CCC(C)(C)C)C(=O)O)cn2)c1] |
| BF039 | 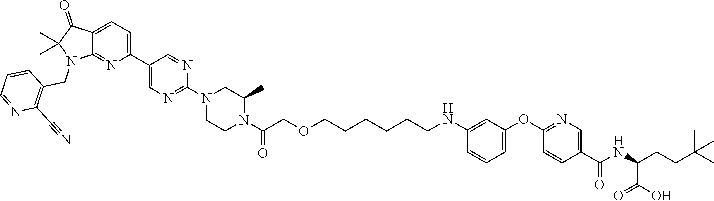 [SMILES: C[C@@H]1CN(c2ncc(-c3ccc4c(n3)N(Cc3cccnc3C#N)C(C)(C)C4=O)cn2)CCN1C(=O)COCCCCCCNc1cccc(Oc2ccc(C(=O)N[C@@H](CCC(C)(C)C)C(=O)O)cn2)c1] |
| BF040 | 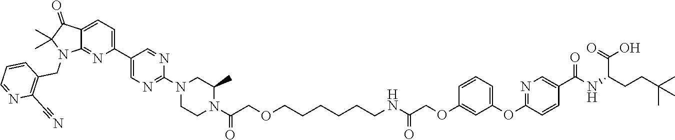 [SMILES: C[C@@H]1CN(c2ncc(-c3ccc4c(n3)N(Cc3cccnc3C#N)C(C)(C)C4=O)cn2)CCN1C(=O)COCCCCCCNC(=O)COc1cccc(Oc2ccc(C(=O)N[C@@H](CCC(C)(C)C)C(=O)O)cn2)c1] |
| BF041 | 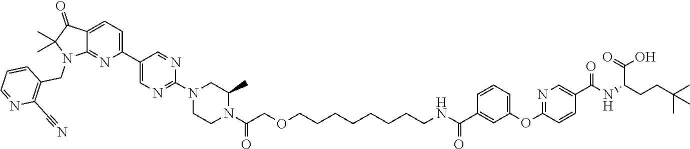 [SMILES: C[C@@H]1CN(c2ncc(-c3ccc4c(n3)N(Cc3cccnc3C#N)C(C)(C)C4=O)cn2)CCN1C(=O)COCCCCCCCCNC(=O)c1cccc(Oc2ccc(C(=O)N[C@@H](CCC(C)(C)C)C(=O)O)cn2)c1] |
| BF042 | 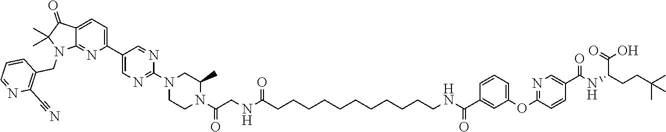 [SMILES: C[C@@H]1CN(c2ncc(-c3ccc4c(n3)N(Cc3cccnc3C#N)C(C)(C)C4=O)cn2)CCN1C(=O)CNC(=O)CCCCCCCCCCCNC(=O)c1cccc(Oc2ccc(C(=O)N[C@@H](CCC(C)(C)C)C(=O)O)cn2)c1] |
| BF043 | 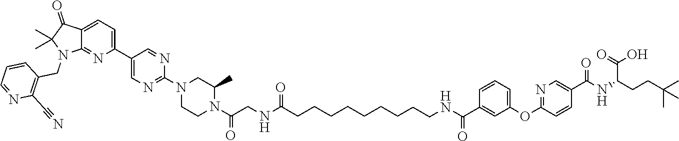 [SMILES: C[C@@H]1CN(c2ncc(-c3ccc4c(n3)N(Cc3cccnc3C#N)C(C)(C)C4=O)cn2)CCN1C(=O)CNC(=O)CCCCCCCCCNC(=O)c1cccc(Oc2ccc(C(=O)N[C@@H](CCC(C)(C)C)C(=O)O)cn2)c1] |
| BF044 | 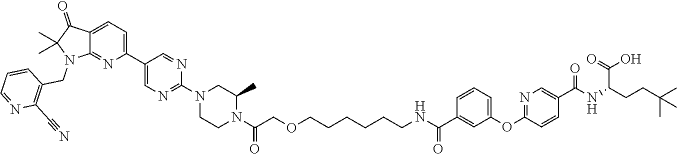 [SMILES: C[C@@H]1CN(c2ncc(-c3ccc4c(n3)N(Cc3cccnc3C#N)C(C)(C)C4=O)cn2)CCN1C(=O)COCCCCCCNC(=O)c1cccc(Oc2ccc(C(=O)N[C@@H](CCC(C)(C)C)C(=O)O)cn2)c1] |
| BF045 | 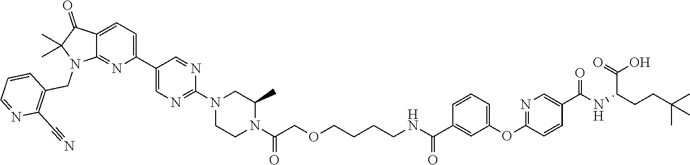 [SMILES: C[C@@H]1CN(c2ncc(-c3ccc4c(n3)N(Cc3cccnc3C#N)C(C)(C)C4=O)cn2)CCN1C(=O)COCCCCNC(=O)c1cccc(Oc2ccc(C(=O)N[C@@H](CCC(C)(C)C)C(=O)O)cn2)c1] |
| BF046 | 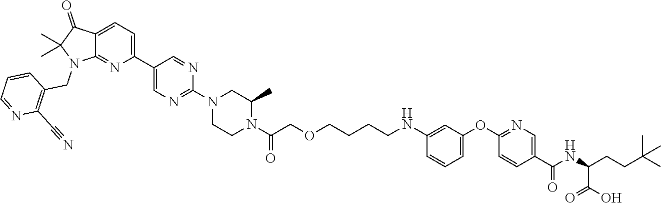 [SMILES: C[C@@H]1CN(c2ncc(-c3ccc4c(n3)N(Cc3cccnc3C#N)C(C)(C)C4=O)cn2)CCN1C(=O)COCCCCNc1cccc(Oc2ccc(C(=O)N[C@@H](CCC(C)(C)C)C(=O)O)cn2)c1] |
| BF047 | 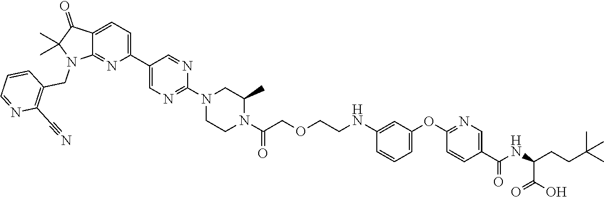 [SMILES: C[C@@H]1CN(c2ncc(-c3ccc4c(n3)N(Cc3cccnc3C#N)C(C)(C)C4=O)cn2)CCN1C(=O)COCCNc1cccc(Oc2ccc(C(=O)N[C@@H](CCC(C)(C)C)C(=O)O)cn2)c1] |
| BF048 | 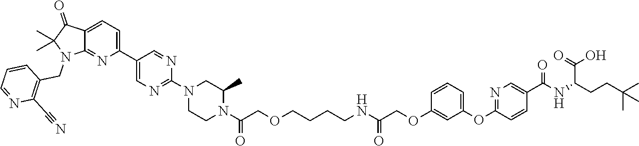 [SMILES: C[C@@H]1CN(c2ncc(-c3ccc4c(n3)N(Cc3cccnc3C#N)C(C)(C)C4=O)cn2)CCN1C(=O)COCCCCNC(=O)COc1cccc(Oc2ccc(C(=O)N[C@@H](CCC(C)(C)C)C(=O)O)cn2)c1] |
| BF049 | 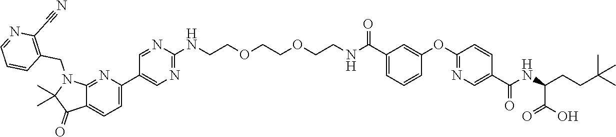 [SMILES: CC(C)(C)CC[C@H](NC(=O)c1ccc(Oc2cccc(C(=O)NCCOCCOCCNc3ncc(-c4ccc5c(n4)N(Cc4cccnc4C#N)C(C)(C)C5=O)cn3)c2)nc1)C(=O)O] |
| BF050 | 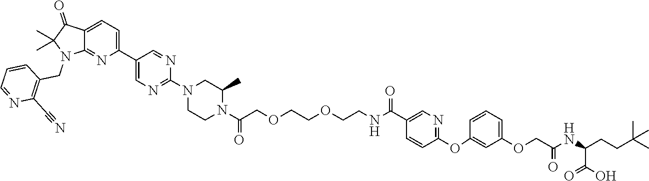 [SMILES: C[C@@H]1CN(c2ncc(-c3ccc4c(n3)N(Cc3cccnc3C#N)C(C)(C)C4=O)cn2)CCN1C(=O)COCCOCCNC(=O)c1ccc(Oc2cccc(OCC(=O)N[C@@H](CCC(C)(C)C)C(=O)O)c2)nc1] |
| BF051 | 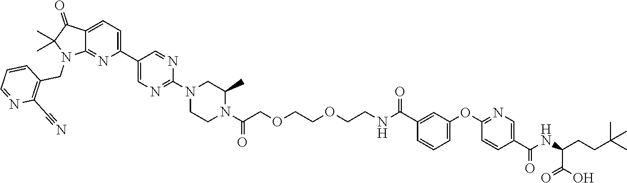 [SMILES: C[C@@H]1CN(c2ncc(-c3ccc4c(n3)N(Cc3cccnc3C#N)C(C)(C)C4=O)cn2)CCN1C(=O)COCCOCCNC(=O)c1cccc(Oc2ccc(C(=O)N[C@@H](CCC(C)(C)C)C(=O)O)cn2)c1] |
| BF052 | 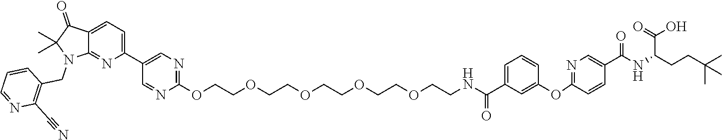 [SMILES: CC(C)(C)CC[C@H](NC(=O)c1ccc(Oc2cccc(C(=O)NCCOCCOCCOCCOCCOc3ncc(-c4ccc5c(n4)N(Cc4cccnc4C#N)C(C)(C)C5=O)cn3)c2)nc1)C(=O)O] |
| BF053 | 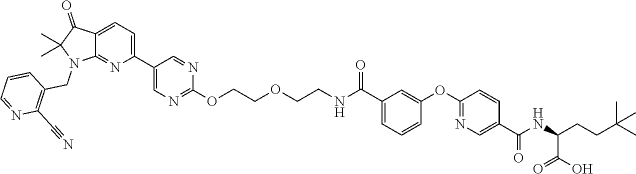 [SMILES: CC(C)(C)CC[C@H](NC(=O)c1ccc(Oc2cccc(C(=O)NCCOCCOc3ncc(-c4ccc5c(n4)N(Cc4cccnc4C#N)C(C)(C)C5=O)cn3)c2)nc1)C(=O)O] |
| BF054 | 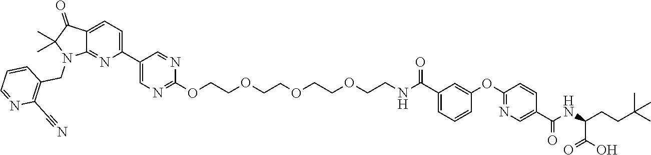 [SMILES: CC(C)(C)CC[C@H](NC(=O)c1ccc(Oc2cccc(C(=O)NCCOCCOCCOCCOc3ncc(-c4ccc5c(n4)N(Cc4cccnc4C#N)C(C)(C)C5=O)cn3)c2)nc1)C(=O)O] |
| BF056 | 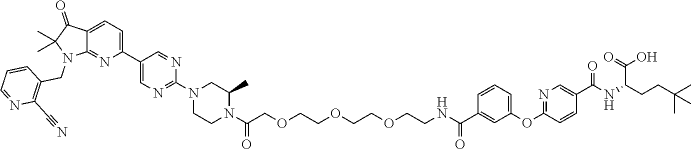 [SMILES: C[C@@H]1CN(c2ncc(-c3ccc4c(n3)N(Cc3cccnc3C#N)C(C)(C)C4=O)cn2)CCN1C(=O)COCCOCCOCCNC(=O)c1cccc(Oc2ccc(C(=O)N[C@@H](CCC(C)(C)C)C(=O)O)cn2)c1] |
| BF058 | 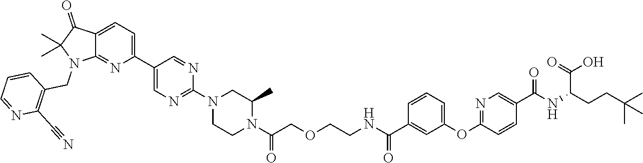 [SMILES: C[C@@H]1CN(c2ncc(-c3ccc4c(n3)N(Cc3cccnc3C#N)C(C)(C)C4=O)cn2)CCN1C(=O)COCCNC(=O)c1cccc(Oc2ccc(C(=O)N[C@@H](CCC(C)(C)C)C(=O)O)cn2)c1] |
| BF059 | 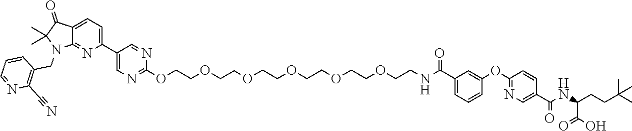 [SMILES: CC(C)(C)CC[C@H](NC(=O)c1ccc(Oc2cccc(C(=O)NCCOCCOCCOCCOCCOCCOc3ncc(-c4ccc5c(n4)N(Cc4cccnc4C#N)C(C)(C)C5=O)cn3)c2)nc1)C(=O)O] |
| BF060 | 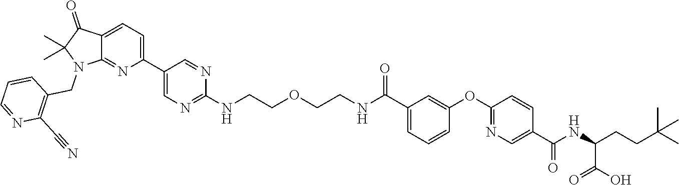 [SMILES: CC(C)(C)CC[C@H](NC(=O)c1ccc(Oc2cccc(C(=O)NCCOCCNc3ncc(-c4ccc5c(n4)N(Cc4cccnc4C#N)C(C)(C)C5=O)cn3)c2)nc1)C(=O)O] |
| BF062 | 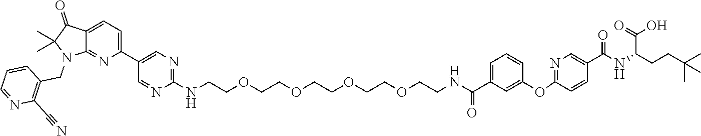 [SMILES: CC(C)(C)CC[C@H](NC(=O)c1ccc(Oc2cccc(C(=O)NCCOCCOCCOCCOCCNc3ncc(-c4ccc5c(n4)N(Cc4cccnc4C#N)C(C)(C)C5=O)cn3)c2)nc1)C(=O)O] |
| BF064 | 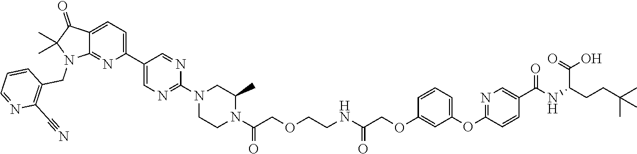 [SMILES: C[C@@H]1CN(c2ncc(-c3ccc4c(n3)N(Cc3cccnc3C#N)C(C)(C)C4=O)cn2)CCN1C(=O)COCCNC(=O)COc1cccc(Oc2ccc(C(=O)N[C@@H](CCC(C)(C)C)C(=O)O)cn2)c1] |
| BF066 | 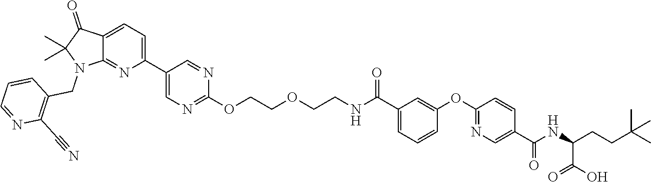 [SMILES: CC(C)(C)CC[C@H](NC(=O)c1ccc(Oc2cccc(C(=O)NCCOCCOc3ncc(-c4ccc5c(n4)N(Cc4cccnc4C#N)C(C)(C)C5=O)cn3)c2)nc1)C(=O)O] |
| BF067 | 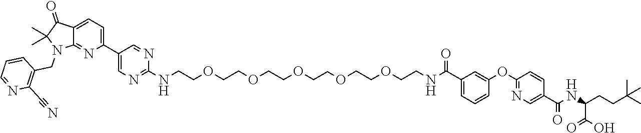 [SMILES: CC(C)(C)CC[C@H](NC(=O)c1ccc(Oc2cccc(C(=O)NCCOCCOCCOCCOCCOCCNc3ncc(-c4ccc5c(n4)N(Cc4cccnc4C#N)C(C)(C)C5=O)cn3)c2)nc1)C(=O)O] |
| BF068 | 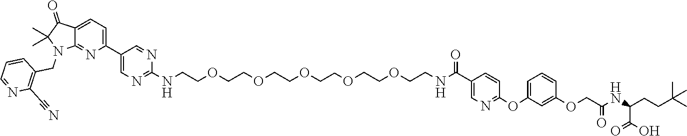 [SMILES: CC(C)(C)CC[C@H](NC(=O)COc1cccc(Oc2ccc(C(=O)NCCOCCOCCOCCOCCOCCNc3ncc(-c4ccc5c(n4)N(Cc4cccnc4C#N)C(C)(C)C5=O)cn3)cn2)c1)C(=O)O] |
| BF070 | 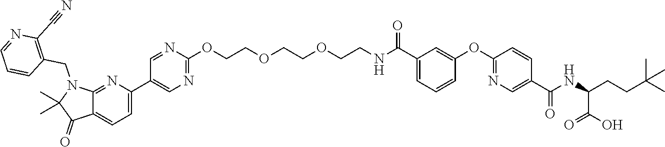 [SMILES: CC(C)(C)CC[C@H](NC(=O)c1ccc(Oc2cccc(C(=O)NCCOCCOCCOc3ncc(-c4ccc5c(n4)N(Cc4cccnc4C#N)C(C)(C)C5=O)cn3)c2)nc1)C(=O)O] |
| BF071 | 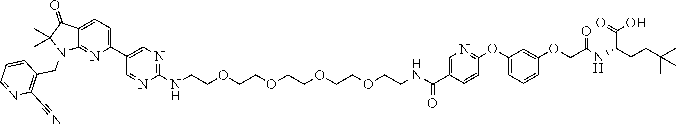 [SMILES: CC(C)(C)CC[C@H](NC(=O)COc1cccc(Oc2ccc(C(=O)NCCOCCOCCOCCOCCNc3ncc(-c4ccc5c(n4)N(Cc4cccnc4C#N)C(C)(C)C5=O)cn3)cn2)c1)C(=O)O] |
| BF072 | 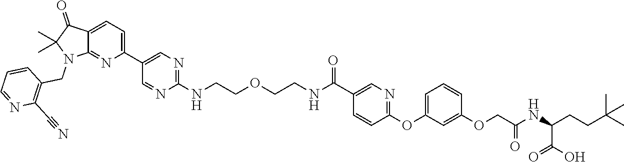 [SMILES: CC(C)(C)CC[C@H](NC(=O)COc1cccc(Oc2ccc(C(=O)NCCOCCNc3ncc(-c4ccc5c(n4)N(Cc4cccnc4C#N)C(C)(C)C5=O)cn3)cn2)c1)C(=O)O] |
| BF073 | 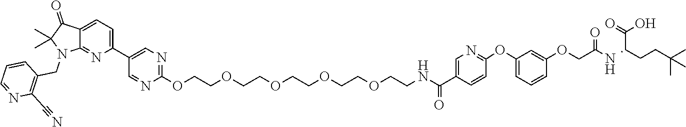 [SMILES: CC(C)(C)CC[C@H](NC(=O)COc1cccc(Oc2ccc(C(=O)NCCOCCOCCOCCOCCOc3ncc(-c4ccc5c(n4)N(Cc4cccnc4C#N)C(C)(C)C5=O)cn3)cn2)c1)C(=O)O] |
| BF074 | 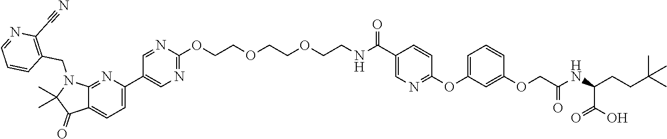 [SMILES: CC(C)(C)CC[C@H](NC(=O)COc1cccc(Oc2ccc(C(=O)NCCOCCOCCOc3ncc(-c4ccc5c(n4)N(Cc4cccnc4C#N)C(C)(C)C5=O)cn3)cn2)c1)C(=O)O] |
| BF075 | 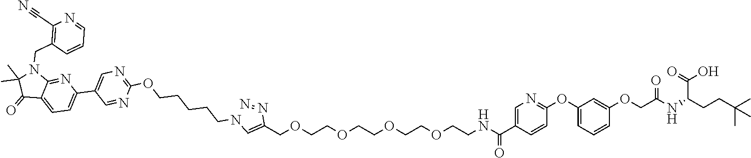 [SMILES: CC(C)(C)CC[C@H](NC(=O)COc1cccc(Oc2ccc(C(=O)NCCOCCOCCOCCOCc3cn(CCCCCOc4ncc(-c5ccc6c(n5)N(Cc5cccnc5C#N)C(C)(C)C6=O)cn4)nn3)cn2)c1)C(=O)O] |
| BF076 | 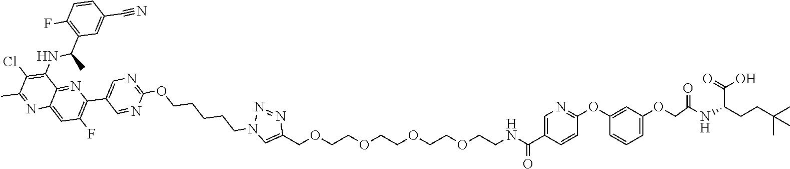 [SMILES: Cc1nc2cc(F)c(-c3cnc(OCCCCCn4cc(COCCOCCOCCOCCNC(=O)c5ccc(Oc6cccc(OCC(=O)N[C@@H](CCC(C)(C)C)C(=O)O)c6)nc5)nn4)nc3)nc2c(N[C@H](C)c2cc(C#N)ccc2F)c1Cl] |
| BF077 | 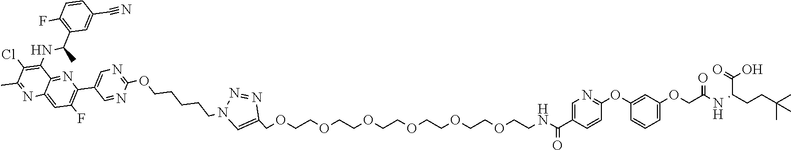 [SMILES: Cc1nc2cc(F)c(-c3cnc(OCCCCCn4cc(COCCOCCOCCOCCOCCOCCNC(=O)c5ccc(Oc6cccc(OCC(=O)N[C@@H](CCC(C)(C)C)C(=O)O)c6)nc5)nn4)nc3)nc2c(N[C@H](C)c2cc(C#N)ccc2F)c1Cl] |
| BF078 | 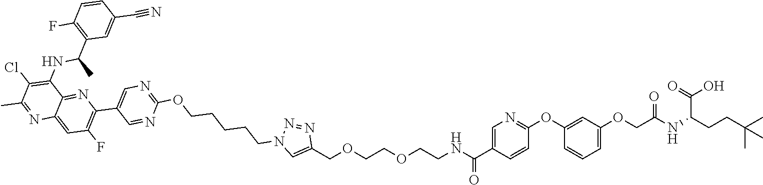 [SMILES: Cc1nc2cc(F)c(-c3cnc(OCCCCCn4cc(COCCOCCNC(=O)c5ccc(Oc6cccc(OCC(=O)N[C@@H](CCC(C)(C)C)C(=O)O)c6)nc5)nn4)nc3)nc2c(N[C@H](C)c2cc(C#N)ccc2F)c1Cl] |
| BF079 | 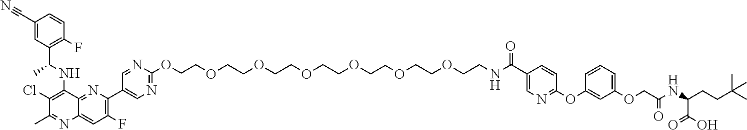 [SMILES: Cc1nc2cc(F)c(-c3cnc(OCCOCCOCCOCCOCCOCCOCCNC(=O)c4ccc(Oc5cccc(OCC(=O)N[C@@H](CCC(C)(C)C)C(=O)O)c5)nc4)nc3)nc2c(N[C@H](C)c2cc(C#N)ccc2F)c1Cl] |
| BF080 | 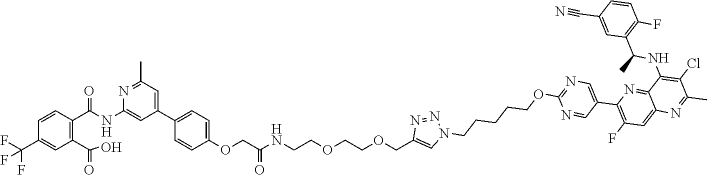 [SMILES: Cc1cc(-c2ccc(OCC(=O)NCCOCCOCc3cn(CCCCCOc4ncc(-c5nc6c(N[C@@H](C)c7cc(C#N)ccc7F)c(Cl)c(C)nc6cc5F)cn4)nn3)cc2)cc(NC(=O)c2ccc(C(F)(F)F)cc2C(=O)O)n1] |
| BF081 | 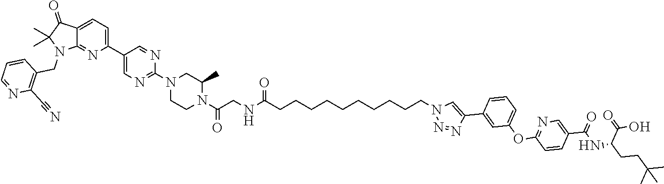 [SMILES: C[C@@H]1CN(c2ncc(-c3ccc4c(n3)N(Cc3cccnc3C#N)C(C)(C)C4=O)cn2)CCN1C(=O)CNC(=O)CCCCCCCCCCn1cc(-c2cccc(Oc3ccc(C(=O)N[C@@H](CCC(C)(C)C)C(=O)O)cn3)c2)nn1] |
| BF082 | 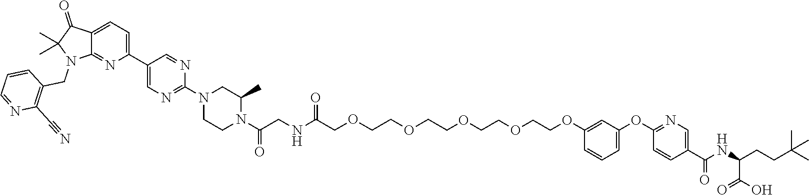 [SMILES: C[C@@H]1CN(c2ncc(-c3ccc4c(n3)N(Cc3cccnc3C#N)C(C)(C)C4=O)cn2)CCN1C(=O)CNC(=O)COCCOCCOCCOCCOc1cccc(Oc2ccc(C(=O)N[C@@H](CCC(C)(C)C)C(=O)O)cn2)c1] |
| BF083 | 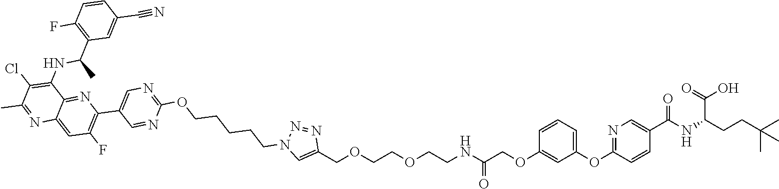 [SMILES: Cc1nc2cc(F)c(-c3cnc(OCCCCCn4cc(COCCOCCNC(=O)COc5cccc(Oc6ccc(C(=O)N[C@@H](CCC(C)(C)C)C(=O)O)cn6)c5)nn4)nc3)nc2c(N[C@H](C)c2cc(C#N)ccc2F)c1Cl] |
| BF084 | 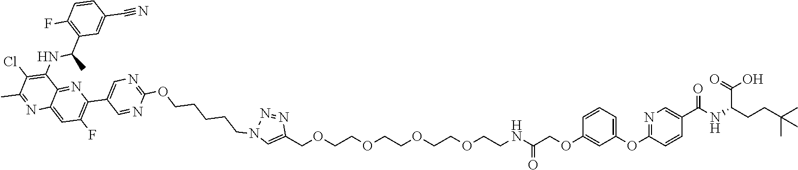 [SMILES: Cc1nc2cc(F)c(-c3cnc(OCCCCCn4cc(COCCOCCOCCOCCNC(=O)COc5cccc(Oc6ccc(C(=O)N[C@@H](CCC(C)(C)C)C(=O)O)cn6)c5)nn4)nc3)nc2c(N[C@H](C)c2cc(C#N)ccc2F)c1Cl] |
| BF085 | 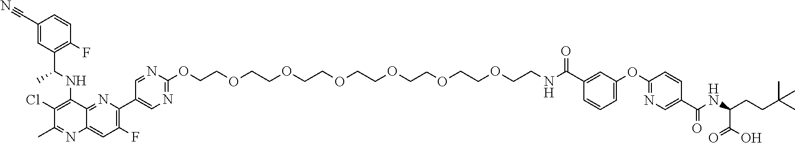 [SMILES: Cc1nc2cc(F)c(-c3cnc(OCCOCCOCCOCCOCCOCCOCCNC(=O)c4cccc(Oc5ccc(C(=O)N[C@@H](CCC(C)(C)C)C(=O)O)cn5)c4)nc3)nc2c(N[C@H](C)c2cc(C#N)ccc2F)c1Cl] |
| BF086 | 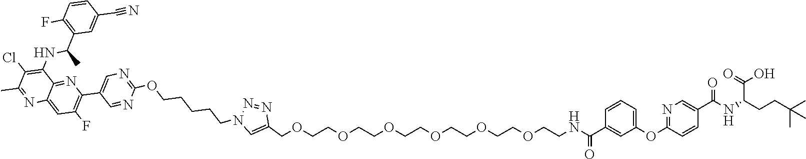 [SMILES: Cc1nc2cc(F)c(-c3cnc(OCCCCCn4cc(COCCOCCOCCOCCOCCOCCNC(=O)c5cccc(Oc6ccc(C(=O)N[C@@H](CCC(C)(C)C)C(=O)O)cn6)c5)nn4)nc3)nc2c(N[C@H](C)c2cc(C#N)ccc2F)c1Cl] |
| BF087 | 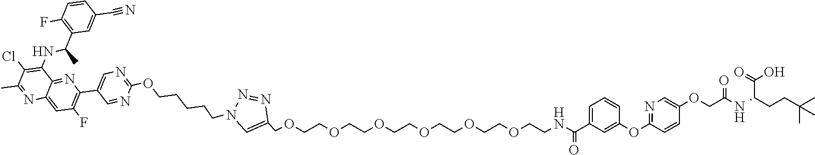 [SMILES: Cc1nc2cc(F)c(-c3cnc(OCCCCCn4cc(COCCOCCOCCOCCOCCOCCNC(=O)c5cccc(Oc6ccc(OCC(=O)N[C@@H](CCC(C)(C)C)C(=O)O)cn6)c5)nn4)nc3)nc2c(N[C@H](C)c2cc(C#N)ccc2F)c1Cl] |
| BF088 | 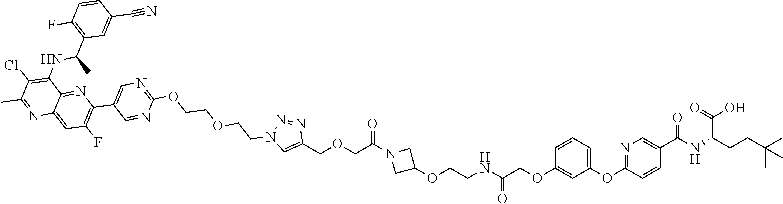 [SMILES: Cc1nc2cc(F)c(-c3cnc(OCCOCCn4cc(COCC(=O)N5CC(OCCNC(=O)COc6cccc(Oc7ccc(C(=O)N[C@@H](CCC(C)(C)C)C(=O)O)cn7)c6)C5)nn4)nc3)nc2c(N[C@H](C)c2cc(C#N)ccc2F)c1Cl] |
| BF089 | 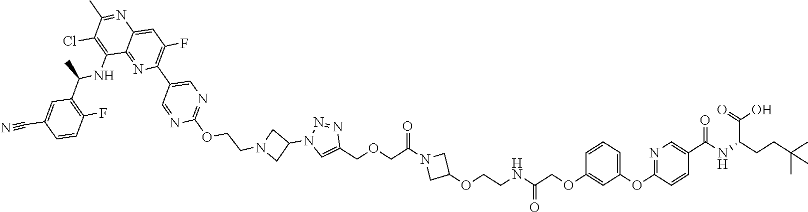 [SMILES: Cc1nc2cc(F)c(-c3cnc(OCCN4CC(n5cc(COCC(=O)N6CC(OCCNC(=O)COc7cccc(Oc8ccc(C(=O)N[C@@H](CCC(C)(C)C)C(=O)O)cn8)c7)C6)nn5)C4)nc3)nc2c(N[C@H](C)c2cc(C#N)ccc2F)c1Cl] |
| BF090 | 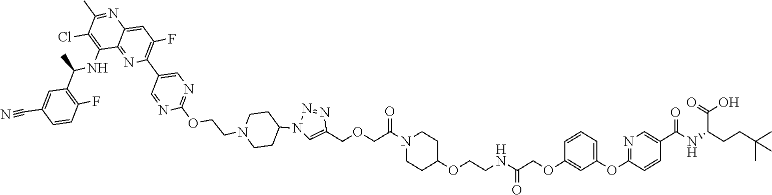 [SMILES: Cc1nc2cc(F)c(-c3cnc(OCCN4CCC(n5cc(COCC(=O)N6CCC(OCCNC(=O)COc7cccc(Oc8ccc(C(=O)N[C@@H](CCC(C)(C)C)C(=O)O)cn8)c7)CC6)nn5)CC4)nc3)nc2c(N[C@H](C)c2cc(C#N)ccc2F)c1Cl] |
| BF091 | 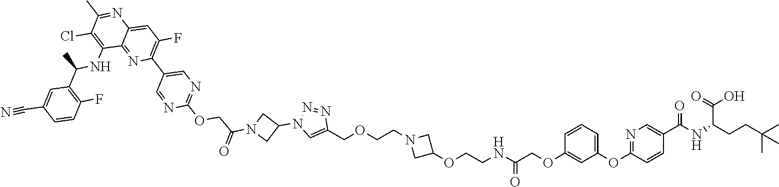 [SMILES: Cc1nc2cc(F)c(-c3cnc(OCC(=O)N4CC(n5cc(COCCN6CC(OCCNC(=O)COc7cccc(Oc8ccc(C(=O)N[C@@H](CCC(C)(C)C)C(=O)O)cn8)c7)C6)nn5)C4)nc3)nc2c(N[C@H](C)c2cc(C#N)ccc2F)c1Cl] |
| BF092 | 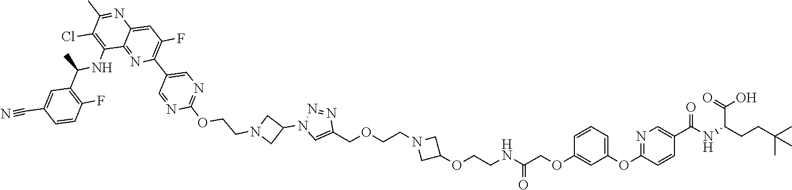 [SMILES: Cc1nc2cc(F)c(-c3cnc(OCCN4CC(n5cc(COCCN6CC(OCCNC(=O)COc7cccc(Oc8ccc(C(=O)N[C@@H](CCC(C)(C)C)C(=O)O)cn8)c7)C6)nn5)C4)nc3)nc2c(N[C@H](C)c2cc(C#N)ccc2F)c1Cl] |
| BF093 | 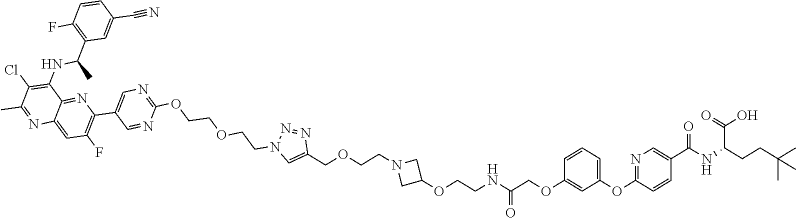 [SMILES: Cc1nc2cc(F)c(-c3cnc(OCCOCCn4cc(COCCN5CC(OCCNC(=O)COc6cccc(Oc7ccc(C(=O)N[C@@H](CCC(C)(C)C)C(=O)O)cn7)c6)C5)nn4)nc3)nc2c(N[C@H](C)c2cc(C#N)ccc2F)c1Cl] |
| BF094 | 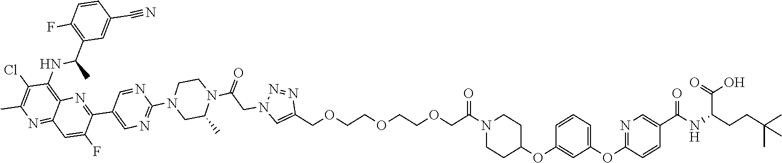 [SMILES: Cc1nc2cc(F)c(-c3cnc(N4CCN(C(=O)Cn5cc(COCCOCCOCC(=O)N6CCC(Oc7cccc(Oc8ccc(C(=O)N[C@@H](CCC(C)(C)C)C(=O)O)cn8)c7)CC6)nn5)[C@H](C)C4)nc3)nc2c(N[C@H](C)c2cc(C#N)ccc2F)c1Cl] |
| BF096 | 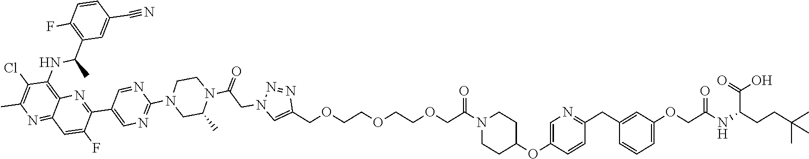 [SMILES: Cc1nc2cc(F)c(-c3cnc(N4CCN(C(=O)Cn5cc(COCCOCCOCC(=O)N6CCC(Oc7ccc(Cc8cccc(OCC(=O)N[C@@H](CCC(C)(C)C)C(=O)O)c8)nc7)CC6)nn5)[C@H](C)C4)nc3)nc2c(N[C@H](C)c2cc(C#N)ccc2F)c1Cl] |
| BF097 | 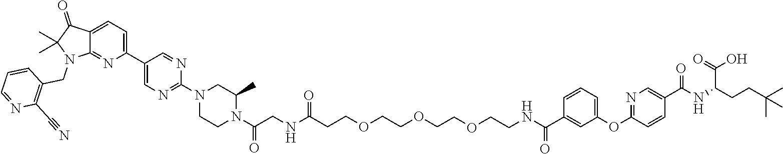 [SMILES: C[C@@H]1CN(c2ncc(-c3ccc4c(n3)N(Cc3cccnc3C#N)C(C)(C)C4=O)cn2)CCN1C(=O)CNC(=O)CCOCCOCCOCCNC(=O)c1cccc(Oc2ccc(C(=O)N[C@@H](CCC(C)(C)C)C(=O)O)cn2)c1] |
| BF098 | 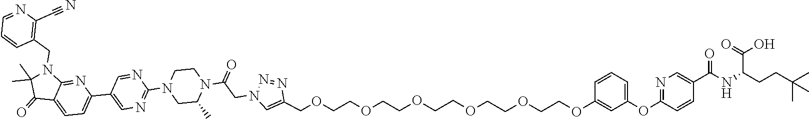 [SMILES: C[C@@H]1CN(c2ncc(-c3ccc4c(n3)N(Cc3cccnc3C#N)C(C)(C)C4=O)cn2)CCN1C(=O)Cn1cc(COCCOCCOCCOCCOCCOc2cccc(Oc3ccc(C(=O)N[C@@H](CCC(C)(C)C)C(=O)O)cn3)c2)nn1] |
| BF099 | 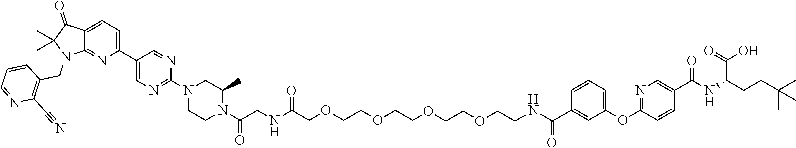 [SMILES: C[C@@H]1CN(c2ncc(-c3ccc4c(n3)N(Cc3cccnc3C#N)C(C)(C)C4=O)cn2)CCN1C(=O)CNC(=O)COCCOCCOCCOCCNC(=O)c1cccc(Oc2ccc(C(=O)N[C@@H](CCC(C)(C)C)C(=O)O)cn2)c1] |
| BF100 | 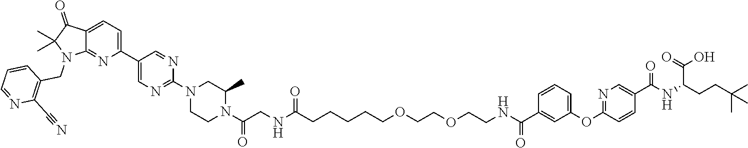 [SMILES: C[C@@H]1CN(c2ncc(-c3ccc4c(n3)N(Cc3cccnc3C#N)C(C)(C)C4=O)cn2)CCN1C(=O)CNC(=O)CCCCCOCCOCCNC(=O)c1cccc(Oc2ccc(C(=O)N[C@@H](CCC(C)(C)C)C(=O)O)cn2)c1] |
| BF101 | 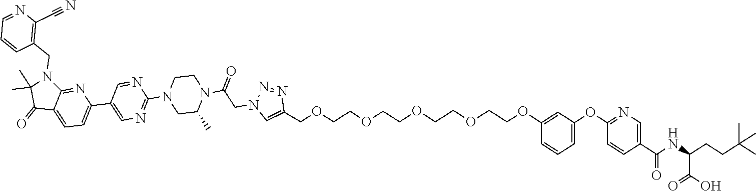 [SMILES: C[C@@H]1CN(c2ncc(-c3ccc4c(n3)N(Cc3cccnc3C#N)C(C)(C)C4=O)cn2)CCN1C(=O)Cn1cc(COCCOCCOCCOCCOc2cccc(Oc3ccc(C(=O)N[C@@H](CCC(C)(C)C)C(=O)O)cn3)c2)nn1] |
| BF102 | 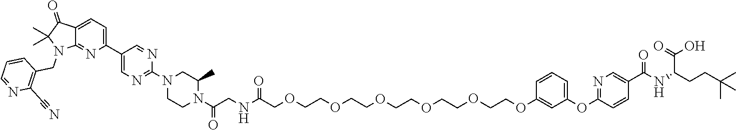 [SMILES: C[C@@H]1CN(c2ncc(-c3ccc4c(n3)N(Cc3cccnc3C#N)C(C)(C)C4=O)cn2)CCN1C(=O)CNC(=O)COCCOCCOCCOCCOCCOc1cccc(Oc2ccc(C(=O)N[C@@H](CCC(C)(C)C)C(=O)O)cn2)c1] |
| BF103 | 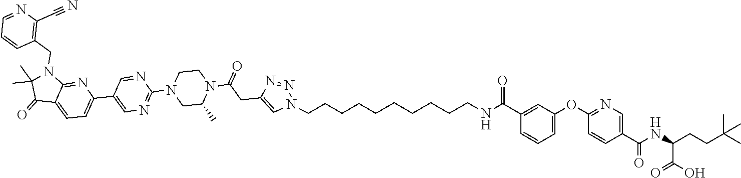 [SMILES: C[C@@H]1CN(c2ncc(-c3ccc4c(n3)N(Cc3cccnc3C#N)C(C)(C)C4=O)cn2)CCN1C(=O)Cc1cn(CCCCCCCCCCNC(=O)c2cccc(Oc3ccc(C(=O)N[C@@H](CCC(C)(C)C)C(=O)O)cn3)c2)nn1] |
| BF104 | 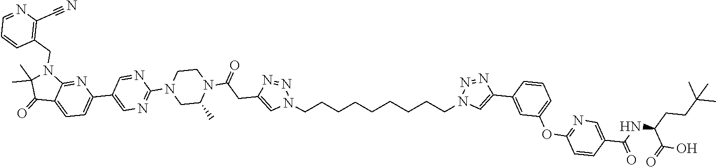 [SMILES: C[C@@H]1CN(c2ncc(-c3ccc4c(n3)N(Cc3cccnc3C#N)C(C)(C)C4=O)cn2)CCN1C(=O)Cc1cn(CCCCCCCCCn2cc(-c3cccc(Oc4ccc(C(=O)N[C@@H](CCC(C)(C)C)C(=O)O)cn4)c3)nn2)nn1] |
| BF105 | 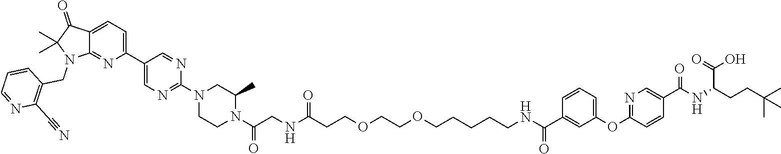 [SMILES: C[C@@H]1CN(c2ncc(-c3ccc4c(n3)N(Cc3cccnc3C#N)C(C)(C)C4=O)cn2)CCN1C(=O)CNC(=O)CCOCCOCCCCCNC(=O)c1cccc(Oc2ccc(C(=O)N[C@@H](CCC(C)(C)C)C(=O)O)cn2)c1] |
| BF106 | 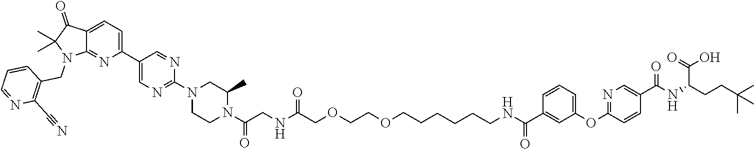 [SMILES: C[C@@H]1CN(c2ncc(-c3ccc4c(n3)N(Cc3cccnc3C#N)C(C)(C)C4=O)cn2)CCN1C(=O)CNC(=O)COCCOCCCCCCNC(=O)c1cccc(Oc2ccc(C(=O)N[C@@H](CCC(C)(C)C)C(=O)O)cn2)c1] |
| BF107 | 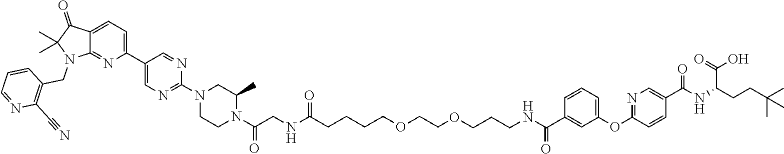 [SMILES: C[C@@H]1CN(c2ncc(-c3ccc4c(n3)N(Cc3cccnc3C#N)C(C)(C)C4=O)cn2)CCN1C(=O)CNC(=O)CCCCOCCOCCCNC(=O)c1cccc(Oc2ccc(C(=O)N[C@@H](CCC(C)(C)C)C(=O)O)cn2)c1] |
| BF108 | 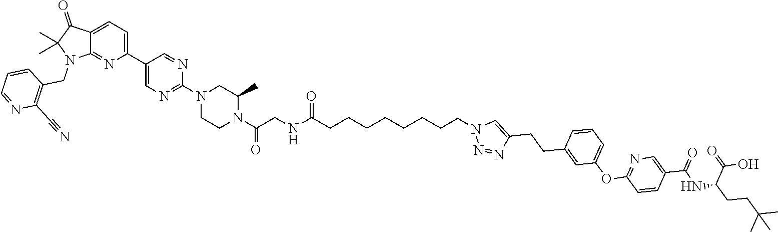 [SMILES: C[C@@H]1CN(c2ncc(-c3ccc4c(n3)N(Cc3cccnc3C#N)C(C)(C)C4=O)cn2)CCN1C(=O)CNC(=O)CCCCCCCCn1cc(CCc2cccc(Oc3ccc(C(=O)N[C@@H](CCC(C)(C)C)C(=O)O)cn3)c2)nn1] |
| BF109 | 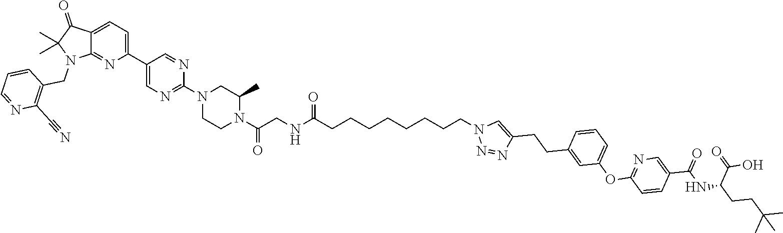 [SMILES: C[C@@H]1CN(c2ncc(-c3ccc4c(n3)N(Cc3cccnc3C#N)C(C)(C)C4=O)cn2)CCN1C(=O)CNC(=O)CCCCCCCCn1cc(CCc2cccc(Oc3ccc(C(=O)N[C@@H](CCC(C)(C)C)C(=O)O)cn3)c2)nn1] |
| BF110 | 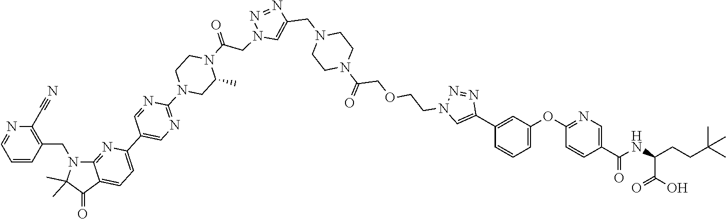 [SMILES: C[C@@H]1CN(c2ncc(-c3ccc4c(n3)N(Cc3cccnc3C#N)C(C)(C)C4=O)cn2)CCN1C(=O)Cn1cc(CN2CCN(C(=O)COCCn3cc(-c4cccc(Oc5ccc(C(=O)N[C@@H](CCC(C)(C)C)C(=O)O)cn5)c4)nn3)CC2)nn1] |
| BF111 | 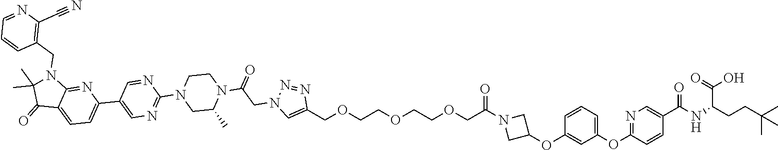 [SMILES: C[C@@H]1CN(c2ncc(-c3ccc4c(n3)N(Cc3cccnc3C#N)C(C)(C)C4=O)cn2)CCN1C(=O)Cn1cc(COCCOCCOCC(=O)N2CC(Oc3cccc(Oc4ccc(C(=O)N[C@@H](CCC(C)(C)C)C(=O)O)cn4)c3)C2)nn1] |
| BF112 | 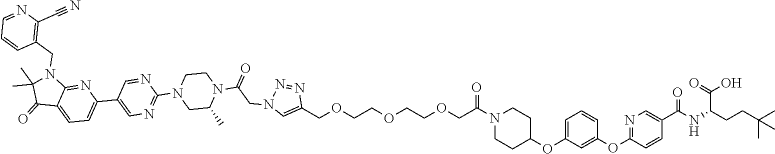 [SMILES: C[C@@H]1CN(c2ncc(-c3ccc4c(n3)N(Cc3cccnc3C#N)C(C)(C)C4=O)cn2)CCN1C(=O)Cn1cc(COCCOCCOCC(=O)N2CCC(Oc3cccc(Oc4ccc(C(=O)N[C@@H](CCC(C)(C)C)C(=O)O)cn4)c3)CC2)nn1] |
| BF113 | 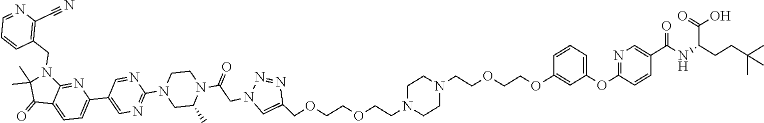 [SMILES: C[C@@H]1CN(c2ncc(-c3ccc4c(n3)N(Cc3cccnc3C#N)C(C)(C)C4=O)cn2)CCN1C(=O)Cn1cc(COCCOCCN2CCN(CCOCCOc3cccc(Oc4ccc(C(=O)N[C@@H](CCC(C)(C)C)C(=O)O)cn4)c3)CC2)nn1] |
| BF114 | 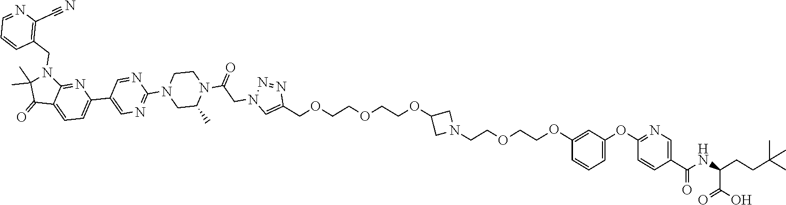 [SMILES: C[C@@H]1CN(c2ncc(-c3ccc4c(n3)N(Cc3cccnc3C#N)C(C)(C)C4=O)cn2)CCN1C(=O)Cn1cc(COCCOCCOC2CN(CCOCCOc3cccc(Oc4ccc(C(=O)N[C@@H](CCC(C)(C)C)C(=O)O)cn4)c3)C2)nn1] |
| BF115 | 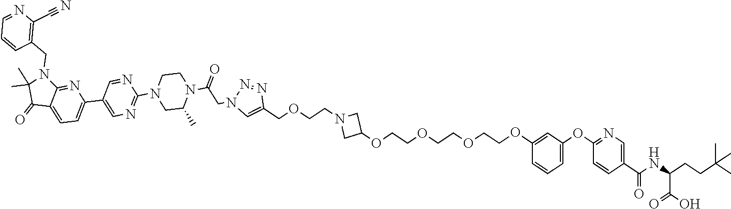 [SMILES: C[C@@H]1CN(c2ncc(-c3ccc4c(n3)N(Cc3cccnc3C#N)C(C)(C)C4=O)cn2)CCN1C(=O)Cn1cc(COCCN2CC(OCCOCCOCCOc3cccc(Oc4ccc(C(=O)N[C@@H](CCC(C)(C)C)C(=O)O)cn4)c3)C2)nn1] |
| BF116 | 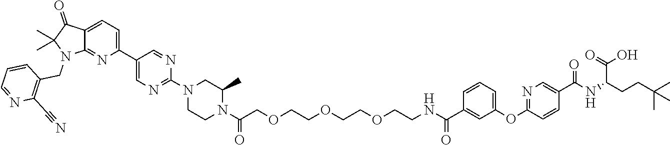 [SMILES: C[C@@H]1CN(c2ncc(-c3ccc4c(n3)N(Cc3cccnc3C#N)C(C)(C)C4=O)cn2)CCN1C(=O)COCCOCCOCCNC(=O)c1cccc(Oc2ccc(C(=O)N[C@@H](CCC(C)(C)C)C(=O)O)cn2)c1] |
| BF117 | 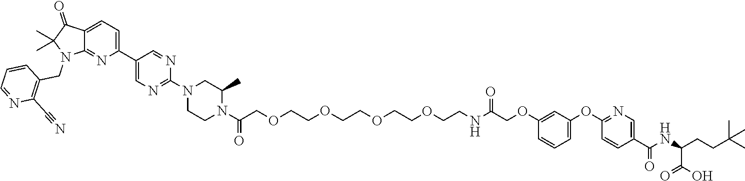 [SMILES: C[C@@H]1CN(c2ncc(-c3ccc4c(n3)N(Cc3cccnc3C#N)C(C)(C)C4=O)cn2)CCN1C(=O)COCCOCCOCCOCCNC(=O)COc1cccc(Oc2ccc(C(=O)N[C@@H](CCC(C)(C)C)C(=O)O)cn2)c1] |
| BF118 | 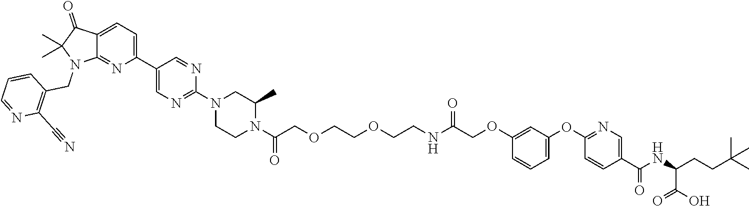 [SMILES: C[C@@H]1CN(c2ncc(-c3ccc4c(n3)N(Cc3cccnc3C#N)C(C)(C)C4=O)cn2)CCN1C(=O)COCCOCCNC(=O)COc1cccc(Oc2ccc(C(=O)N[C@@H](CCC(C)(C)C)C(=O)O)cn2)c1] |
| BF119 | 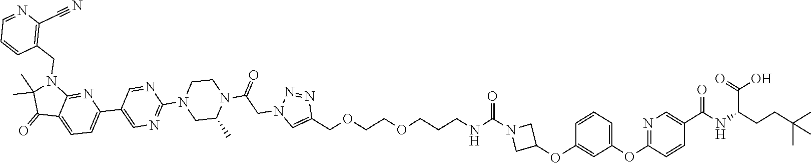 [SMILES: C[C@@H]1CN(c2ncc(-c3ccc4c(n3)N(Cc3cccnc3C#N)C(C)(C)C4=O)cn2)CCN1C(=O)Cn1cc(COCCOCCCNC(=O)N2CC(Oc3cccc(Oc4ccc(C(=O)N[C@@H](CCC(C)(C)C)C(=O)O)cn4)c3)C2)nn1] |
| BF120 | 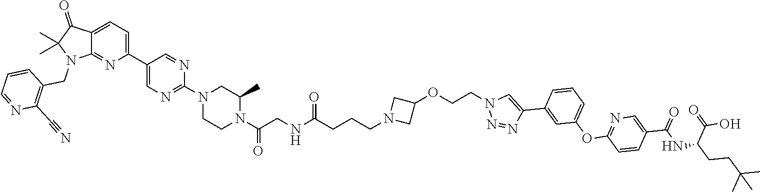 [SMILES: C[C@@H]1CN(c2ncc(-c3ccc4c(n3)N(Cc3cccnc3C#N)C(C)(C)C4=O)cn2)CCN1C(=O)CNC(=O)CCCN1CC(OCCn2cc(-c3cccc(Oc4ccc(C(=O)N[C@@H](CCC(C)(C)C)C(=O)O)cn4)c3)nn2)C1] |
| BF121 | 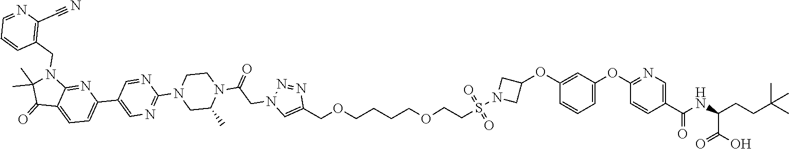 [SMILES: C[C@@H]1CN(c2ncc(-c3ccc4c(n3)N(Cc3cccnc3C#N)C(C)(C)C4=O)cn2)CCN1C(=O)Cn1cc(COCCCCOCCS(=O)(=O)N2CC(Oc3cccc(Oc4ccc(C(=O)N[C@@H](CCC(C)(C)C)C(=O)O)cn4)c3)C2)nn1] |
| BF122 | 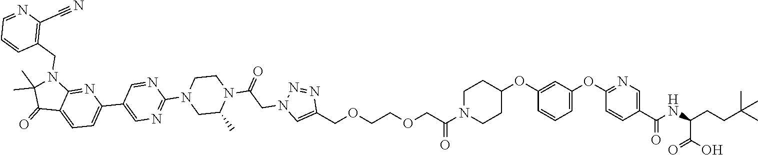 [SMILES: C[C@@H]1CN(c2ncc(-c3ccc4c(n3)N(Cc3cccnc3C#N)C(C)(C)C4=O)cn2)CCN1C(=O)Cn1cc(COCCOCC(=O)N2CCC(Oc3cccc(Oc4ccc(C(=O)N[C@@H](CCC(C)(C)C)C(=O)O)cn4)c3)CC2)nn1] |
| BF123 | 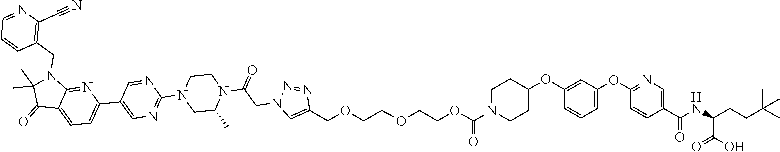 [SMILES: C[C@@H]1CN(c2ncc(-c3ccc4c(n3)N(Cc3cccnc3C#N)C(C)(C)C4=O)cn2)CCN1C(=O)Cn1cc(COCCOCCOC(=O)N2CCC(Oc3cccc(Oc4ccc(C(=O)N[C@@H](CCC(C)(C)C)C(=O)O)cn4)c3)CC2)nn1] |
| BF124 | 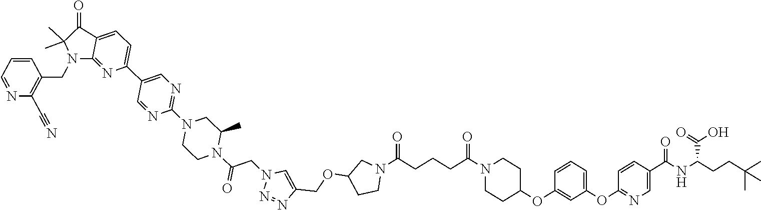 [SMILES: C[C@@H]1CN(c2ncc(-c3ccc4c(n3)N(Cc3cccnc3C#N)C(C)(C)C4=O)cn2)CCN1C(=O)Cn1cc(COC2CCN(C(=O)CCCC(=O)N3CCC(Oc4cccc(Oc5ccc(C(=O)N[C@@H](CCC(C)(C)C)C(=O)O)cn5)c4)CC3)C2)nn1] |
| BF125 | 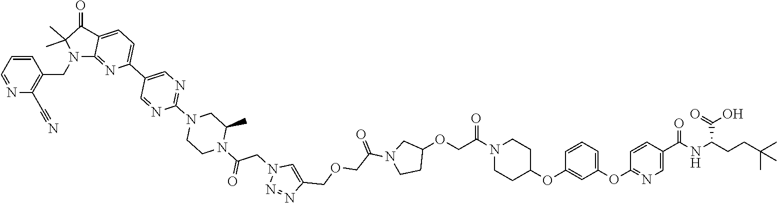 [SMILES: C[C@@H]1CN(c2ncc(-c3ccc4c(n3)N(Cc3cccnc3C#N)C(C)(C)C4=O)cn2)CCN1C(=O)Cn1cc(COCC(=O)N2CCC(OCC(=O)N3CCC(Oc4cccc(Oc5ccc(C(=O)N[C@@H](CCC(C)(C)C)C(=O)O)cn5)c4)CC3)C2)nn1] |
| BF126 | 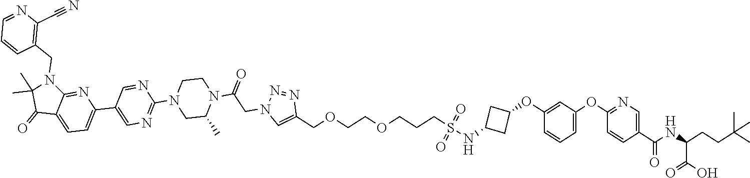 [SMILES: C[C@@H]1CN(c2ncc(-c3ccc4c(n3)N(Cc3cccnc3C#N)C(C)(C)C4=O)cn2)CCN1C(=O)Cn1cc(COCCOCCCS(=O)(=O)N[C@H]2C[C@@H](Oc3cccc(Oc4ccc(C(=O)N[C@@H](CCC(C)(C)C)C(=O)O)cn4)c3)C2)nn1] |
| BF127 | 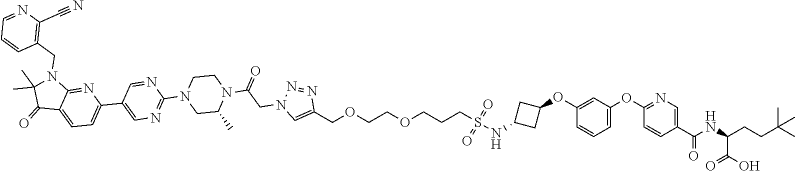 [SMILES: C[C@@H]1CN(c2ncc(-c3ccc4c(n3)N(Cc3cccnc3C#N)C(C)(C)C4=O)cn2)CCN1C(=O)Cn1cc(COCCOCCCS(=O)(=O)N[C@H]2C[C@H](Oc3cccc(Oc4ccc(C(=O)N[C@@H](CCC(C)(C)C)C(=O)O)cn4)c3)C2)nn1] |
| BF128 | 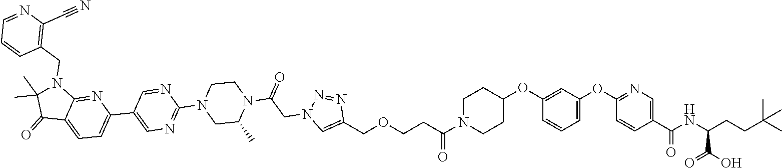 [SMILES: C[C@@H]1CN(c2ncc(-c3ccc4c(n3)N(Cc3cccnc3C#N)C(C)(C)C4=O)cn2)CCN1C(=O)Cn1cc(COCCC(=O)N2CCC(Oc3cccc(Oc4ccc(C(=O)N[C@@H](CCC(C)(C)C)C(=O)O)cn4)c3)CC2)nn1] |
| BF129 | 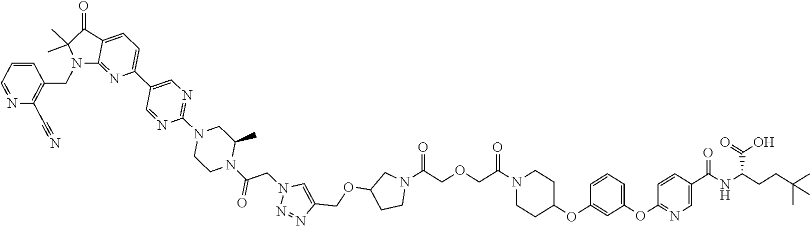 [SMILES: C[C@@H]1CN(c2ncc(-c3ccc4c(n3)N(Cc3cccnc3C#N)C(C)(C)C4=O)cn2)CCN1C(=O)Cn1cc(COC2CCN(C(=O)COCC(=O)N3CCC(Oc4cccc(Oc5ccc(C(=O)N[C@@H](CCC(C)(C)C)C(=O)O)cn5)c4)CC3)C2)nn1] |
| BF130 | 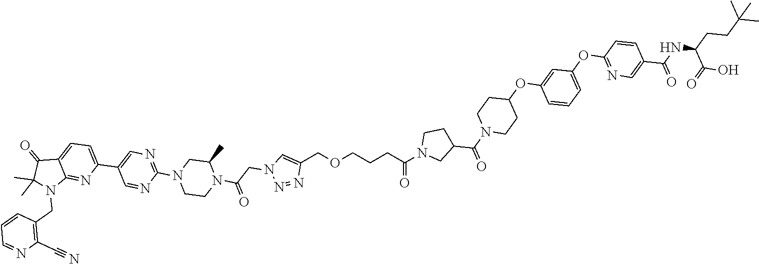 [SMILES: C[C@@H]1CN(c2ncc(-c3ccc4c(n3)N(Cc3cccnc3C#N)C(C)(C)C4=O)cn2)CCN1C(=O)Cn1cc(COCCCC(=O)N2CCC(C(=O)N3CCC(Oc4cccc(Oc5ccc(C(=O)N[C@@H](CCC(C)(C)C)C(=O)O)cn5)c4)CC3)C2)nn1] |
| BF131 | 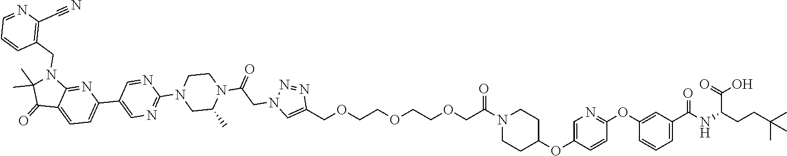 [SMILES: C[C@@H]1CN(c2ncc(-c3ccc4c(n3)N(Cc3cccnc3C#N)C(C)(C)C4=O)cn2)CCN1C(=O)Cn1cc(COCCOCCOCC(=O)N2CCC(Oc3ccc(Oc4cccc(C(=O)N[C@@H](CCC(C)(C)C)C(=O)O)c4)nc3)CC2)nn1] |
| BF132 | 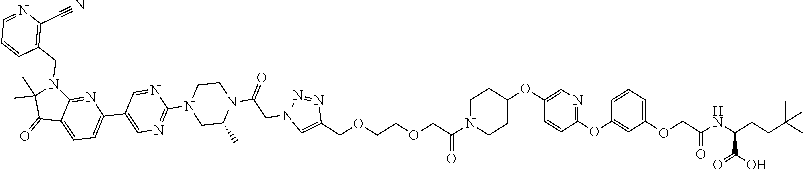 [SMILES: C[C@@H]1CN(c2ncc(-c3ccc4c(n3)N(Cc3cccnc3C#N)C(C)(C)C4=O)cn2)CCN1C(=O)Cn1cc(COCCOCC(=O)N2CCC(Oc3ccc(Oc4cccc(OCC(=O)N[C@@H](CCC(C)(C)C)C(=O)O)c4)nc3)CC2)nn1] |
| BF133 | 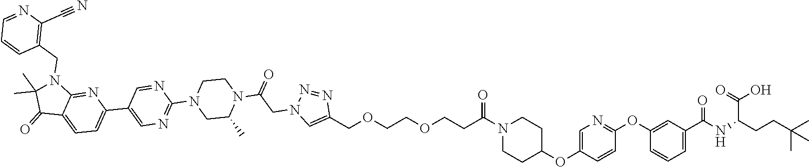 [SMILES: C[C@@H]1CN(c2ncc(-c3ccc4c(n3)N(Cc3cccnc3C#N)C(C)(C)C4=O)cn2)CCN1C(=O)Cn1cc(COCCOCCC(=O)N2CCC(Oc3ccc(Oc4cccc(C(=O)N[C@@H](CCC(C)(C)C)C(=O)O)c4)nc3)CC2)nn1] |
| BF134 | 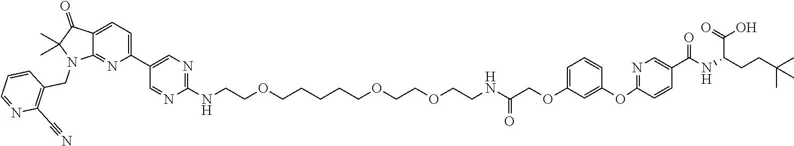 [SMILES: CC(C)(C)CC[C@H](NC(=O)c1ccc(Oc2cccc(OCC(=O)NCCOCCOCCCCCOCCNc3ncc(-c4ccc5c(n4)N(Cc4cccnc4C#N)C(C)(C)C5=O)cn3)c2)nc1)C(=O)O] |
| BF135 | 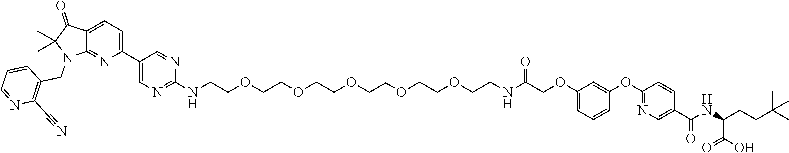 [SMILES: CC(C)(C)CC[C@H](NC(=O)c1ccc(Oc2cccc(OCC(=O)NCCOCCOCCOCCOCCOCCNc3ncc(-c4ccc5c(n4)N(Cc4cccnc4C#N)C(C)(C)C5=O)cn3)c2)nc1)C(=O)O] |
| BF136 | 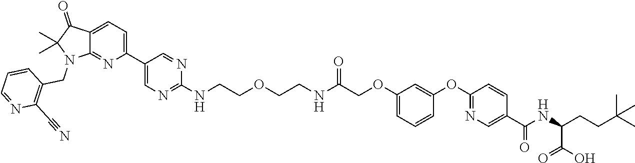 [SMILES: CC(C)(C)CC[C@H](NC(=O)c1ccc(Oc2cccc(OCC(=O)NCCOCCNc3ncc(-c4ccc5c(n4)N(Cc4cccnc4C#N)C(C)(C)C5=O)cn3)c2)nc1)C(=O)O] |
| BF137 | 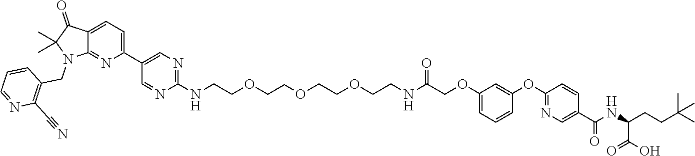 [SMILES: CC(C)(C)CC[C@H](NC(=O)c1ccc(Oc2cccc(OCC(=O)NCCOCCOCCOCCNc3ncc(-c4ccc5c(n4)N(Cc4cccnc4C#N)C(C)(C)C5=O)cn3)c2)nc1)C(=O)O] |
| BF138 | 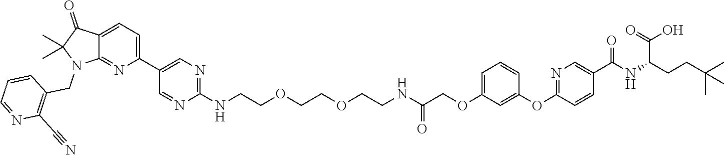 [SMILES: CC(C)(C)CC[C@H](NC(=O)c1ccc(Oc2cccc(OCC(=O)NCCOCCOCCNc3ncc(-c4ccc5c(n4)N(Cc4cccnc4C#N)C(C)(C)C5=O)cn3)c2)nc1)C(=O)O] |
| BF139 | 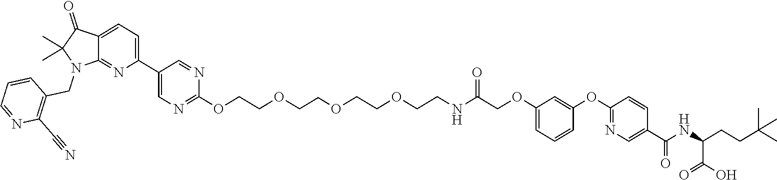 [SMILES: CC(C)(C)CC[C@H](NC(=O)c1ccc(Oc2cccc(OCC(=O)NCCOCCOCCOCCOc3ncc(-c4ccc5c(n4)N(Cc4cccnc4C#N)C(C)(C)C5=O)cn3)c2)nc1)C(=O)O] |
| BF140 | 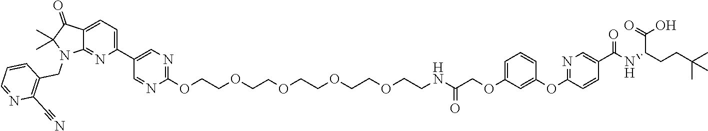 [SMILES: CC(C)(C)CC[C@H](NC(=O)c1ccc(Oc2cccc(OCC(=O)NCCOCCOCCOCCOCCOc3ncc(-c4ccc5c(n4)N(Cc4cccnc4C#N)C(C)(C)C5=O)cn3)c2)nc1)C(=O)O] |
| BF141 | 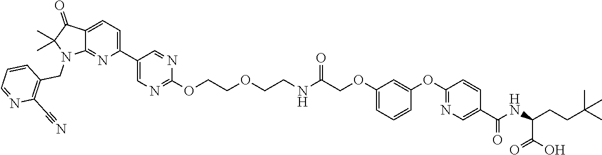 [SMILES: CC(C)(C)CC[C@H](NC(=O)c1ccc(Oc2cccc(OCC(=O)NCCOCCOc3ncc(-c4ccc5c(n4)N(Cc4cccnc4C#N)C(C)(C)C5=O)cn3)c2)nc1)C(=O)O] |
| BF142 | 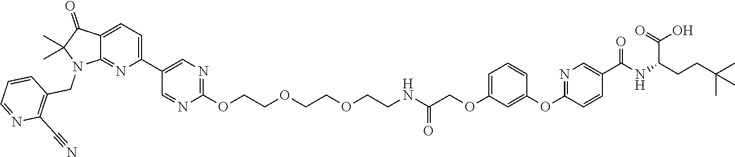 [SMILES: CC(C)(C)CC[C@H](NC(=O)c1ccc(Oc2cccc(OCC(=O)NCCOCCOCCOc3ncc(-c4ccc5c(n4)N(Cc4cccnc4C#N)C(C)(C)C5=O)cn3)c2)nc1)C(=O)O] |
| BF143 | 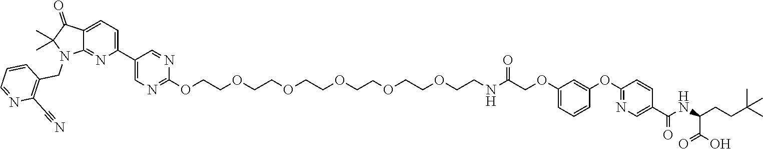 [SMILES: CC(C)(C)CC[C@H](NC(=O)c1ccc(Oc2cccc(OCC(=O)NCCOCCOCCOCCOCCOCCOc3ncc(-c4ccc5c(n4)N(Cc4cccnc4C#N)C(C)(C)C5=O)cn3)c2)nc1)C(=O)O] |
| BF144 | 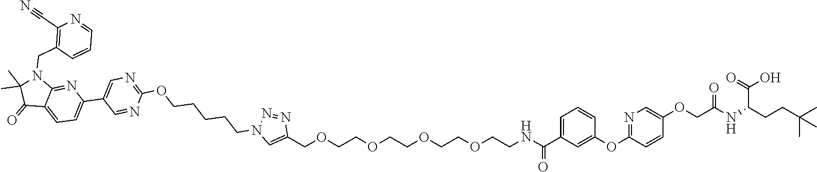 [SMILES: CC(C)(C)CC[C@H](NC(=O)COc1ccc(Oc2cccc(C(=O)NCCOCCOCCOCCOCc3cn(CCCCCOc4ncc(-c5ccc6c(n5)N(Cc5cccnc5C#N)C(C)(C)C6=O)cn4)nn3)c2)nc1)C(=O)O] |
| BF145 | 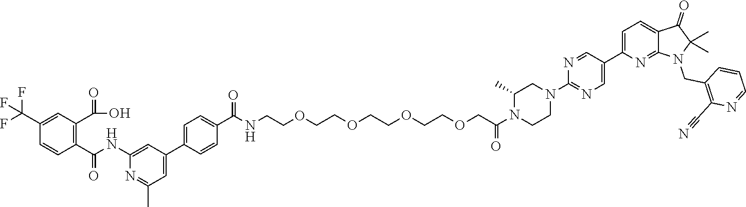 [SMILES: Cc1cc(-c2ccc(C(=O)NCCOCCOCCOCCOCC(=O)N3CCN(c4ncc(-c5ccc6c(n5)N(Cc5cccnc5C#N)C(C)(C)C6=O)cn4)C[C@H]3C)cc2)cc(NC(=O)c2ccc(C(F)(F)F)cc2C(=O)O)n1] |
| BF146 | 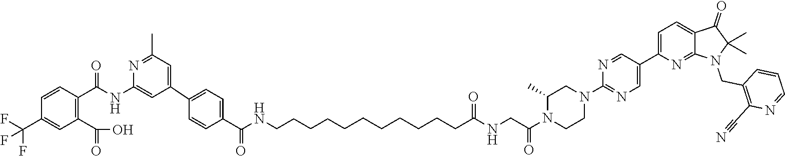 [SMILES: Cc1cc(-c2ccc(C(=O)NCCCCCCCCCCCC(=O)NCC(=O)N3CCN(c4ncc(-c5ccc6c(n5)N(Cc5cccnc5C#N)C(C)(C)C6=O)cn4)C[C@H]3C)cc2)cc(NC(=O)c2ccc(C(F)(F)F)cc2C(=O)O)n1] |
| BF147 | 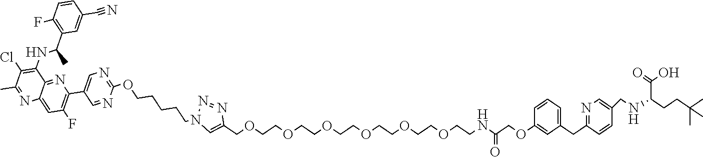 [SMILES: Cc1nc2cc(F)c(-c3cnc(OCCCCCn4cc(COCCOCCOCCOCCOCCOCCNC(=O)COc5cccc(Cc6ccc(CN[C@@H](CCC(C)(C)C)C(=O)O)cn6)c5)nn4)nc3)nc2c(N[C@H](C)c2cc(C#N)ccc2F)c1Cl] |
| BF148 | 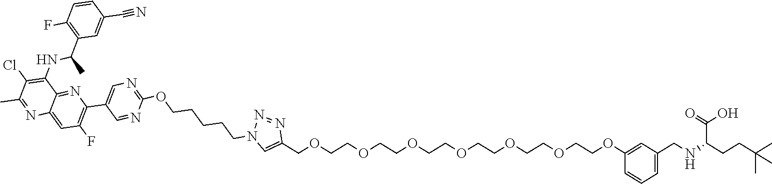 [SMILES: Cc1nc2cc(F)c(-c3cnc(OCCCCCn4cc(COCCOCCOCCOCCOCCOCCOc5cccc(CN[C@@H](CCC(C)(C)C)C(=O)O)c5)nn4)nc3)nc2c(N[C@H](C)c2cc(C#N)ccc2F)c1Cl] |
| BF149 | 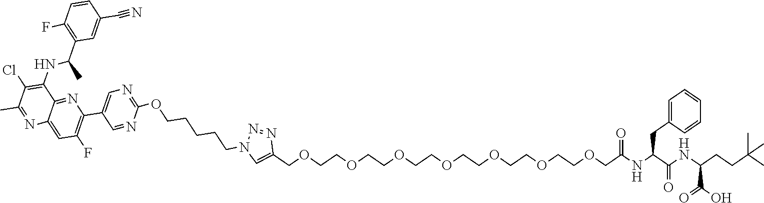 [SMILES: Cc1nc2cc(F)c(-c3cnc(OCCCCCn4cc(COCCOCCOCCOCCOCCOCCOCC(=O)N[C@@H](Cc5ccccc5)C(=O)N[C@@H](CCC(C)(C)C)C(=O)O)nn4)nc3)nc2c(N[C@H](C)c2cc(C#N)ccc2F)c1Cl] |

TABLE B

| ID. | Structure |
|---|---|
| SB001 | 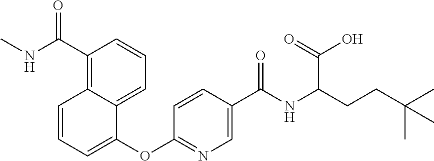 [SMILES: CNC(=O)c1cccc2c(Oc3ccc(C(=O)NC(CCC(C)(C)C)C(=O)O)cn3)cccc12] |
| SB002 | 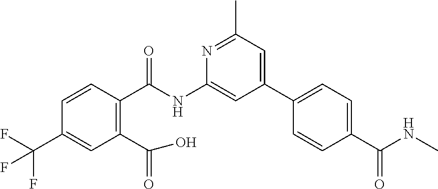 [SMILES: CNC(=O)c1ccc(-c2cc(C)nc(NC(=O)c3ccc(C(F)(F)F)cc3C(=O)O)c2)cc1] |
| SB003 | 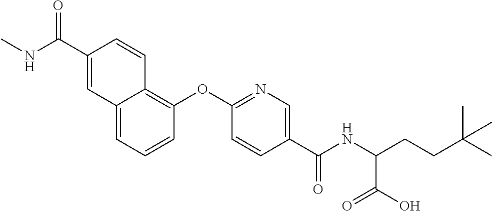 [SMILES: CNC(=O)c1ccc2c(Oc3ccc(C(=O)NC(CCC(C)(C)C)C(=O)O)cn3)cccc2c1] |
| SB004 | 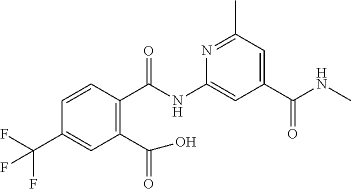 [SMILES: CNC(=O)c1cc(C)nc(NC(=O)c2ccc(C(F)(F)F)cc2C(=O)O)c1] |
| SB005 | 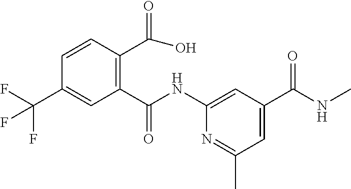 [SMILES: CNC(=O)c1cc(C)nc(NC(=O)c2cc(C(F)(F)F)ccc2C(=O)O)c1] |
| SB006 | 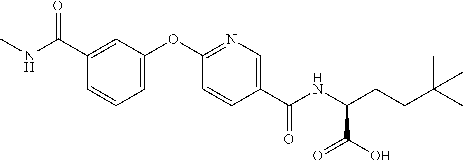 [SMILES: CNC(=O)c1cccc(Oc2ccc(C(=O)N[C@@H](CCC(C)(C)C)C(=O)O)cn2)c1] |
| SB007 | 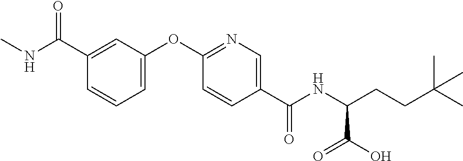 [SMILES: CNC(=O)c1cccc(Oc2ccc(C(=O)N[C@@H](CCC(C)(C)C)C(=O)O)cn2)c1] |
| SB008 | 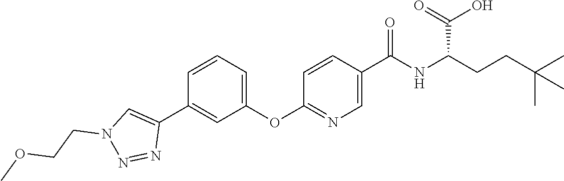 [SMILES: COCCn1cc(-c2cccc(Oc3ccc(C(=O)N[C@@H](CCC(C)(C)C)C(=O)O)cn3)c2)nn1] |
| SB009 | 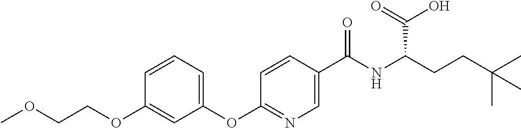 [SMILES: COCCOc1cccc(Oc2ccc(C(=O)N[C@@H](CCC(C)(C)C)C(=O)O)cn2)c1] |
| SB010 | 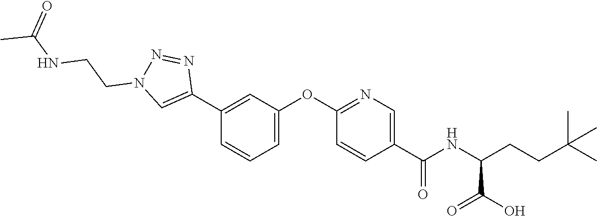 [SMILES: CC(=O)NCCn1cc(-c2cccc(Oc3ccc(C(=O)N[C@@H](CCC(C)(C)C)C(=O)O)cn3)c2)nn1] |
| SB011 | 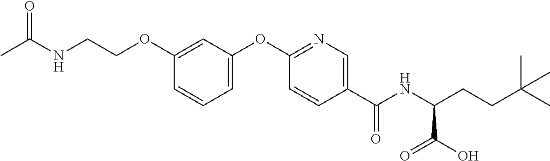 [SMILES: CC(=O)NCCOc1cccc(Oc2ccc(C(=O)N[C@@H](CCC(C)(C)C)C(=O)O)cn2)c1] |
| SB012 | 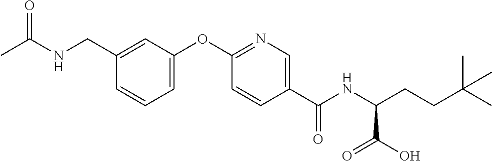 [SMILES: CC(=O)NCc1cccc(Oc2ccc(C(=O)N[C@@H](CCC(C)(C)C)C(=O)O)cn2)c1] |
| SB013 | 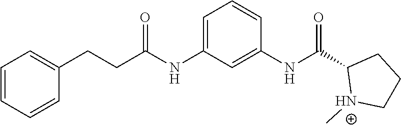 [SMILES: C[NH+]1CCC[C@H]1C(=O)Nc1cccc(NC(=O)CCc2ccccc2)c1] |
| SB014 | 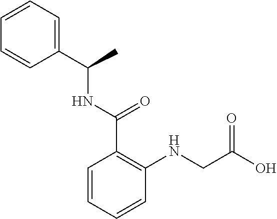 [SMILES: C[C@@H](NC(=O)c1ccccc1NCC(=O)O)c1ccccc1] |
| SB015 | 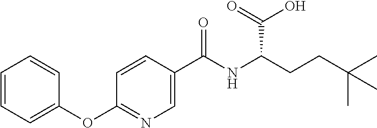 [SMILES: CC(C)(C)CC[C@H](NC(=O)c1ccc(Oc2ccccc2)nc1)C(=O)O] |
| SB016 | 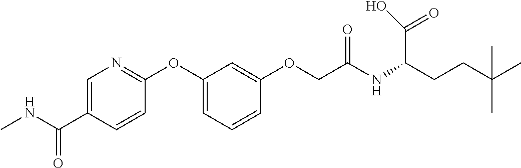 [SMILES: CNC(=O)c1ccc(Oc2cccc(OCC(=O)N[C@@H](CCC(C)(C)C)C(=O)O)c2)nc1] |

TABLE C

| ID. | Compound |
|---|---|
| TF001 | 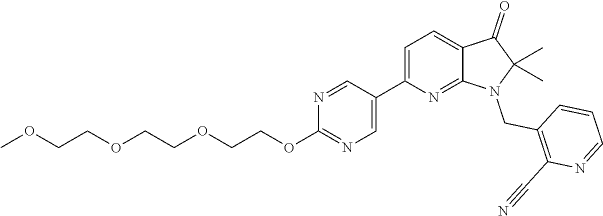 [SMILES: COCCOCCOCCOc1ncc(-c2ccc3c(n2)N(Cc2cccnc2C#N)C(C)(C)C3=O)cn1] |
| TF002 | 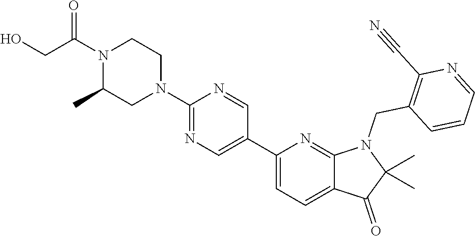 [SMILES: C[C@@H]1CN(c2ncc(-c3ccc4c(n3)N(Cc3cccnc3C#N)C(C)(C)C4=O)cn2)CCN1C(=O)CO] |
| TF003 | 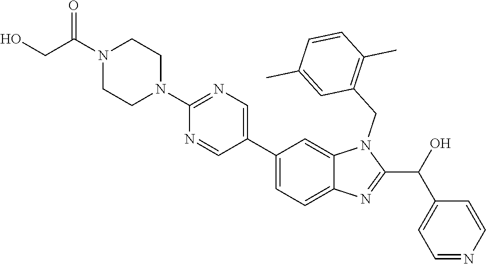 [SMILES: Cc1ccc(C)c(Cn2c(C(O)c3ccncc3)nc3ccc(-c4cnc(N5CCN(C(=O)CO)CC5)nc4)cc32)c1] |
| TF004 | 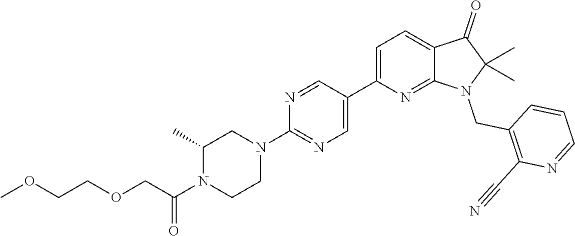 [SMILES: COCCOCC(=O)N1CCN(c2ncc(-c3ccc4c(n3)N(Cc3cccnc3C#N)C(C)(C)C4=O)cn2)C[C@H]1C] |
| TF005 | 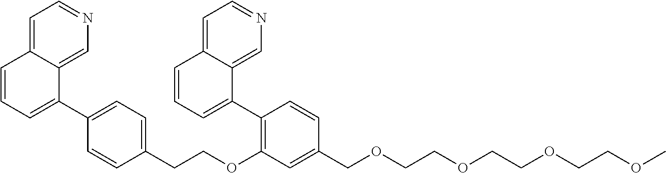 [SMILES: COCCOCCOCCOCc1ccc(-c2cccc3ccncc23)c(OCCc2ccc(-c3cccc4ccncc34)cc2)c1] |
| TF006 | 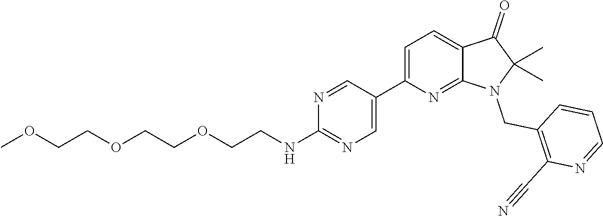 [SMILES: COCCOCCOCCNc1ncc(-c2ccc3c(n2)N(Cc2cccnc2C#N)C(C)(C)C3=O)cn1] |
| TF007 | 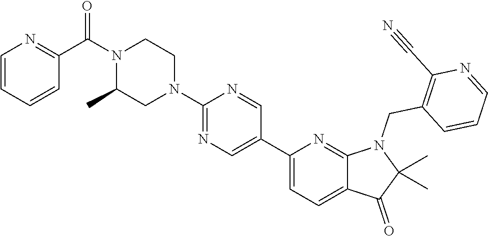 [SMILES: C[C@@H]1CN(c2ncc(-c3ccc4c(n3)N(Cc3cccnc3C#N)C(C)(C)C4=O)cn2)CCN1C(=O)c1ccccn1] |
| TF008 | 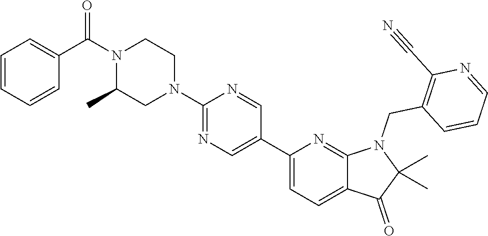 [SMILES: C[C@@H]1CN(c2ncc(-c3ccc4c(n3)N(Cc3cccnc3C#N)C(C)(C)C4=O)cn2)CCN1C(=O)c1ccccc1] |
| TF009 | 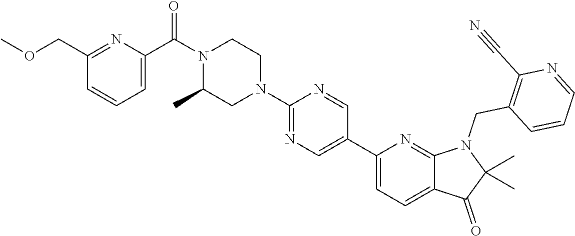 [SMILES: COCc1cccc(C(=O)N2CCN(c3ncc(-c4ccc5c(n4)N(Cc4cccnc4C#N)C(C)(C)C5=O)cn3)C[C@H]2C)n1] |
| TF010 | 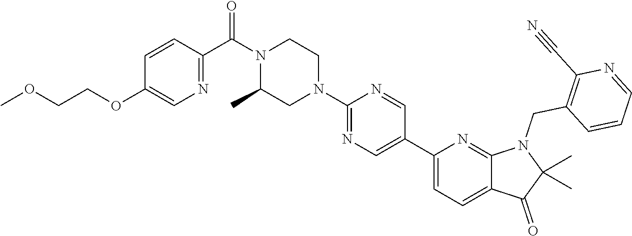 [SMILES: COCCOc1ccc(C(=O)N2CCN(c3ncc(-c4ccc5c(n4)N(Cc4cccnc4C#N)C(C)(C)C5=O)cn3)C[C@H]2C)nc1] |
| TF011 | 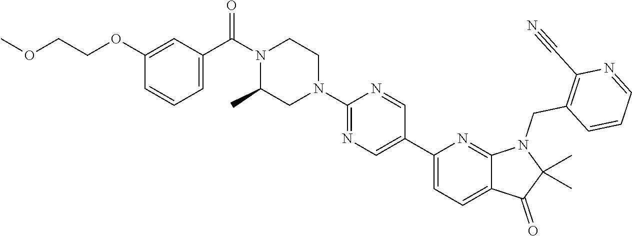 [SMILES: COCCOc1cccc(C(=O)N2CCN(c3ncc(-c4ccc5c(n4)N(Cc4cccnc4C#N)C(C)(C)C5=O)cn3)C[C@H]2C)c1] |
| TF012 | 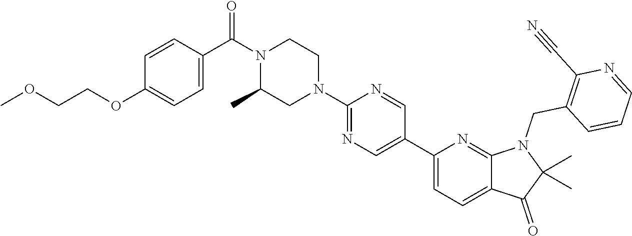 [SMILES: COCCOc1ccc(C(=O)N2CCN(c3ncc(-c4ccc5c(n4)N(Cc4cccnc4C#N)C(C)(C)C5=O)cn3)C[C@H]2C)cc1] |
| TF013 | 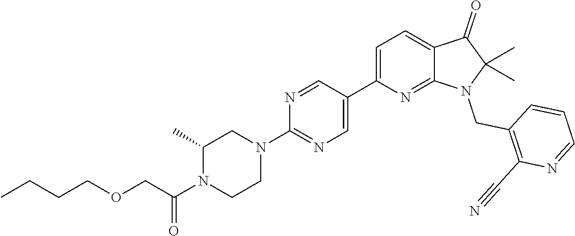 [SMILES: CCCCOCC(=O)N1CCN(c2ncc(-c3ccc4c(n3)N(Cc3cccnc3C#N)C(C)(C)C4=O)cn2)C[C@H]1C] |
| TF014 | 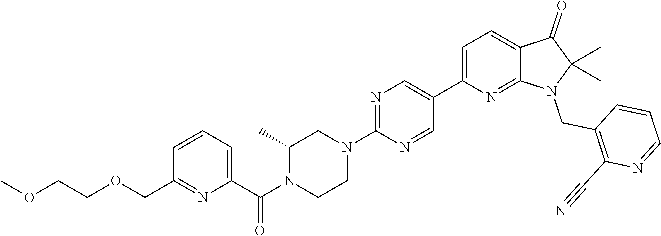 [SMILES: COCCOCc1cccc(C(=O)N2CCN(c3ncc(-c4ccc5c(n4)N(Cc4cccnc4C#N)C(C)(C)C5=O)cn3)C[C@H]2C)n1] |
| TF015 | 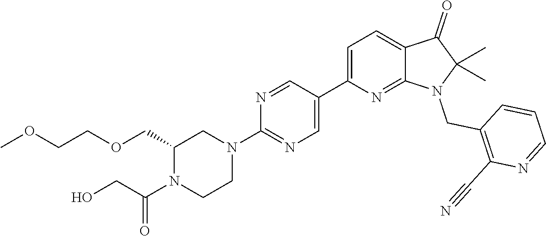 [SMILES: COCCOC[C@@H]1CN(c2ncc(-c3ccc4c(n3)N(Cc3cccnc3C#N)C(C)(C)C4=O)cn2)CCN1C(=O)CO] |
| TF016 | 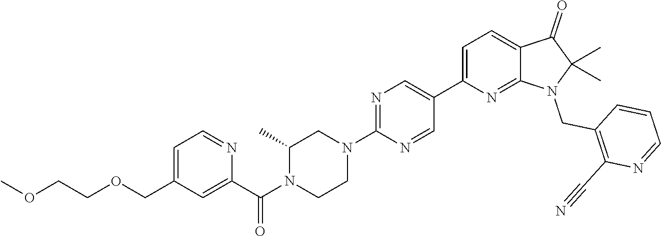 [SMILES: COCCOCc1ccnc(C(=O)N2CCN(c3ncc(-c4ccc5c(n4)N(Cc4cccnc4C#N)C(C)(C)C5=O)cn3)C[C@H]2C)c1] |
| TF017 | 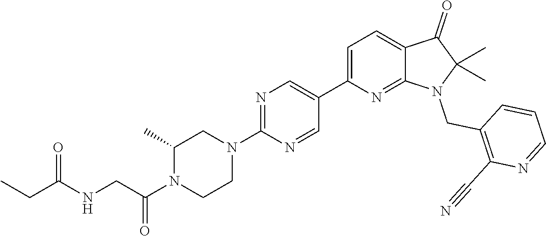 [SMILES: CCC(=O)NCC(=O)N1CCN(c2ncc(-c3ccc4c(n3)N(Cc3cccnc3C#N)C(C)(C)C4=O)cn2)C[C@H]1C] |
| TF018 | 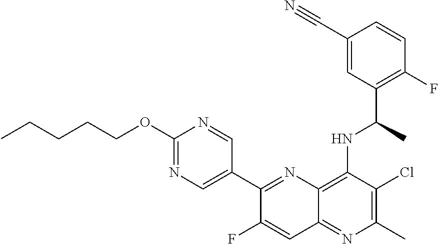 [SMILES: CCCCCOc1ncc(-c2nc3c(N[C@H](C)c4cc(C#N)ccc4F)c(Cl)c(C)nc3cc2F)cn1] |
| TF019 | 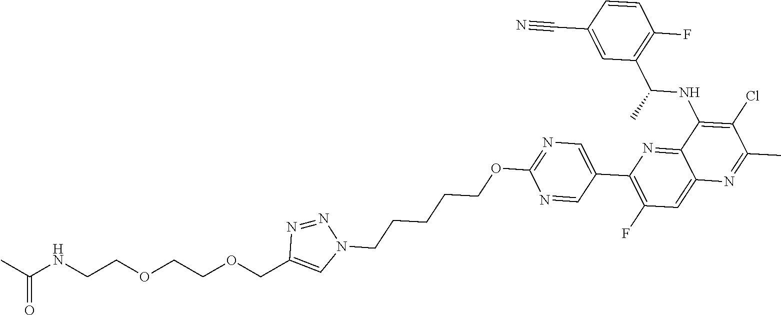 [SMILES: CC(=O)NCCOCCOCc1cn(CCCCCOc2ncc(-c3nc4c(N[C@H](C)c5cc(C#N)ccc5F)c(Cl)c(C)nc4cc3F)cn2)nn1] |
| TF020 | 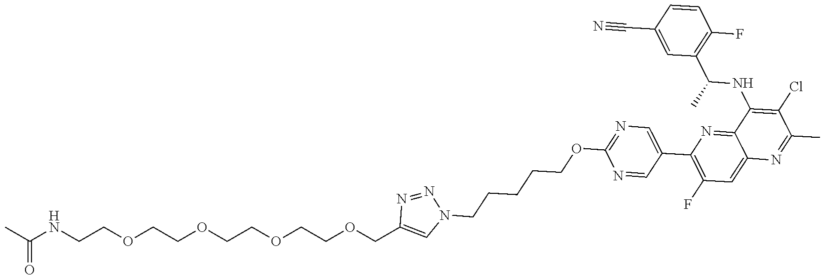 [SMILES: CC(=O)NCCOCCOCCOCCOCc1cn(CCCCCOc2ncc(-c3nc4c(N[C@H](C)c5cc(C#N)ccc5F)c(Cl)c(C)nc4cc3F)cn2)nn1] |
| TF021 | 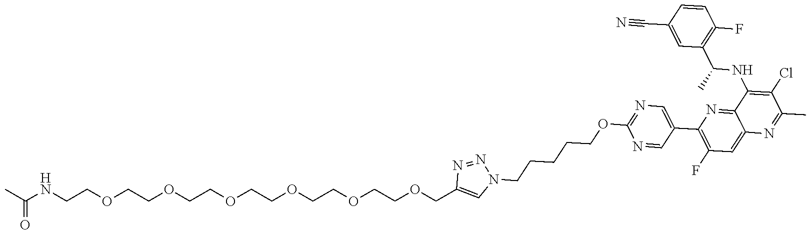 [SMILES: CC(=O)NCCOCCOCCOCCOCCOCCOCc1cn(CCCCCOc2ncc(-c3nc4c(N[C@H](C)c5cc(C#N)ccc5F)c(Cl)c(C)nc4cc3F)cn2)nn1] |
| TF022 | 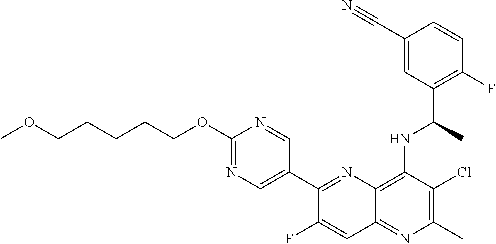 [SMILES: COCCCCCOc1ncc(-c2nc3c(N[C@H](C)c4cc(C#N)ccc4F)c(Cl)c(C)nc3cc2F)cn1] |

| ID. | Name |
|---|---|
| BF001 | (2S)-2-[[6-[3-[2-[2-[2-(2,4-dinitroanilino)ethoxy]ethoxy] |
| | ethylcarbamoyl]phenoxy]pyridine-3-carbonyl]amino]- |
| | 5,5-dimethyl-hexanoic acid |
| BF002 | (2S)-2-[[6-[3-[2-[2-[2-[2-(2,4- |
| | dinitroanilino)ethoxy]ethoxy]ethoxy]ethyl- |
| | carbamoyl]phenoxy]pyridine-3- |
| | carbonyl]amino]-5,5-dimethyl-hexanoic acid |
| BF003 | (2S)-2-[[6-[3-[2-[2-[2-[2-[2-(2,4- |
| | dinitroanilino)ethoxy]ethoxy]ethoxy]ethoxy] |
| | ethylcarbamoyl]phenoxy]pyridine-3-carbonyl]amino]- |
| | 5,5-dimethyl-hexanoic acid |
| BF004 | (2S)-2-[[6-[3-[2-[2-[2-[2-[2-[2-(2,4- |
| | dinitroanilino)ethoxy]ethoxy]ethoxy]ethoxy] |
| | ethoxy]ethoxy]phenoxy]pyridine-3-carbonyl]amino]- |
| | 5,5-dimethyl-hexanoic acid |
| BF005 | (2S)-2-[[6-[5-[3-[3-[2-[2-[2-(2,4- |
| | dinitroanilino)ethoxy]ethoxy]ethoxy] |
| | propanoylamino]propylcarbamoyl]- |
| | 1-naphthyl]oxy]pyridine-3- |
| | carbonyllamino]-5,5-dimethyl-hexanoic acid |
| BF006 | (2S)-2-[[6-[3-[2-[2-[2-[2-[2-[2-[2-[2-(2,4- |
| | dinitroanilino)ethoxy]ethoxy]ethoxy]ethoxy]ethoxy] |
| | ethoxy]ethoxy]ethylcarbamoyl]phenoxy]pyridine-3- |
| | carbonyl]amino]-5,5-dimethyl-hexanoic acid |
| BF007 | (2S)-2-[[6-[3-[2-[2-[2-[2-(2,4-dinitroanilino)ethoxy] |
| | ethoxy]ethoxy]ethoxy]phenoxy]pyridine-3- |
| | carbonyl]amino]-5,5-dimethyl-hexanoic acid |
| BF008 | (2S)-2-[[6-[3-[2-[2-[2-[2-[2-[2-[2-(2,4- |
| | dinitroanilino)ethoxy]ethoxy]ethoxy]ethoxy] |
| | ethoxy]ethoxy]ethylcarbamoyl]phenoxy]pyridine-3- |
| | carbonyl]amino]-5,5-dimethyl-hexanoic acid |
| BF009 | (2S)-2-[[6-[5-[3-[3-[2-[2-(2,4-dinitroanilino) |
| | ethoxy]ethoxy]propanoylamino]propylcarbamoyl]-1- |
| | naphthyl]oxy]pyridine-3-carbonyl]amino]- |
| | 5,5-dimethyl-hexanoic acid |
| BF010 | (2S)-2-[[6-[5-[3-[3-[2-[2-[2-[2-(2,4- |
| | dinitroanilino)ethoxy]ethoxy]ethoxy]ethoxy] |
| | propanoylamino]propylcarbamoyl]-1-naphthyl]oxy] |
| | pyridine-3-carbonyl]amino]-5,5-dimethyl-hexanoic acid |
| BF011 | (2S)-2-[[6-[3-[2-[2-[2-[2-[2-[2-(2,4- |
| | dinitroanilino)ethoxy]ethoxy]ethoxy]ethoxy] |
| | ethoxy]ethylcarbamoyl]phenoxy]pyridine-3- |
| | carbonyl]amino]-5,5-dimethyl-hexanoic acid |
| BF012 | (2S)-2-[[6-[3-[2-[2-[2-[2-[2-[2-[2-[2-(2,4- |
| | dinitroanilino)ethoxy]ethoxy]ethoxy]ethoxy]ethoxy] |
| | ethoxy]ethoxy]ethoxy]phenoxy]pyridine-3- |
| | carbonyl]amino]-5,5-dimethyl-hexanoic acid |
| BF013 | (2S)-2-[[6-[3-[2-[2-[2-(2,4-dinitroanilino)ethoxy] |
| | ethoxy]ethoxy]phenoxy]pyridine-3-carbonyl]amino]- |
| | 5,5-dimethyl-hexanoic acid |
| BF014 | (2S)-2-[[6-[3-[2-[2-[2-[2-[2-[2-[2-(2,4- |
| | dinitroanilino)ethoxy]ethoxy]ethoxy] |
| | ethoxy]ethoxy]ethoxy]ethoxy]phenoxy] pyridine-3- |
| | carbonyl]amino]-5,5-dimethyl-hexanoic acid |
| BF015 | (2S)-2-[[6-[5-[3-[3-[2-(2,4-dinitroanilino) |
| | ethoxy]propanoylamino]propylcarbamoyl]- |
| | 1-naphthyl]oxy]pyridine-3- |
| | carbonyl]amino]-5,5-dimethyl-hexanoic acid |
| BF016 | (2S)-2-[[6-[[5-[3-[3-(2,4- |
| | dinitroanilino)propanoylamino] propylcarbamoyl]- |
| | 1-naphthyl]oxy]pyridine-3-carbonyl]amino]- |
| | 5,5-dimethyl-hexanoic acid |
| BF017 | (2S)-2-[[6-[3-[2-[2-[2-[2-[2-[2-[2-[2-[5-[(3aS,4S,6aR)- |
| | 2-oxo-1,3,3a,4,6,6a-hexahydrothieno[3,4-d]imidazol-4- |
| | yl]pentanoylamino]ethoxy]ethoxy]ethoxy]ethoxy]ethoxy] |
| | ethoxy]ethoxy]ethoxy]phenoxy]pyridine- |
| | 3-carbonyl]amino]-5,5-dimethyl- |
| | hexanoic acid |
| BF018 | (2S)-2-[[6-[3-[2-[2-[2-[2-[2-[2-[2-[2-[2-[5-[(3aS,4S,6aR)- |
| | 2-oxo-1,3,3a,4,6,6a-hexahydrothieno[3,4-d]imidazol-4- |
| | yl]pentanoylamino]ethoxy]ethoxy]ethoxy]ethoxy]ethoxy] |
| | ethoxy]ethoxy]ethoxy]ethylcarbamoyl]phenoxy]pyridine-3- |
| | carbonyl]amino]-5,5-dimethyl-hexanoic acid |
| BF019 | (2S)-2-[[6-[3-[2-[2-[2-[2-[2-[2-[2-[5- |
| | [(3aS,4S,6aR)-2-oxo-1,3,3a,4,6,6a- |
| | hexahydrothieno[3,4-d]imidazol-4-yl]pentanoyl- |
| | amino]ethoxy]ethoxy]ethoxy]ethoxy]ethoxy]ethoxy] |
| | ethoxy]phenoxy]pyridine-3-carbonyl]amino]- |
| | 5,5-dimethyl-hexanoic acid |
| BF020 | (2S)-2-[[6-[[5-[3-[3-[2-[2-[2-[2-[5-[(3aS,4S,6aR)-2- |
| | oxo-1,3,3a,4,6,6a-hexahydrothieno[3,4-d]imidazol-4- |
| | yl]pentanoylamino]ethoxy]ethoxy]ethoxy]ethoxy] |
| | propanoylamino]propylcarbamoyl]-1-naphthyl]oxy]pyridine-3- |
| | carbonyl]amino]-5,5-dimethyl-hexanoic acid |
| BF021 | (2S)-2-[[6-[3-[2-[2-[2-[2-[2-[2-[2-[2-[5-[(3aS,4S,6aR)-2-oxo- |
| | 1,3,3a,4,6,6a-hexahydrothieno[3,4-d]imidazol-4- |
| | yl]pentanoylamino]ethoxy]ethoxy]ethoxy]ethoxy] |
| | ethoxy]ethoxy]ethoxy]ethylcarbamoyl]phenoxy]pyridine-3- |
| | carbonyl]amino]-5,5-dimethyl-hexanoic acid |
| BF023 | (2S)-2-[[6-[3-[2-[2-[2-[2-[2-[2-[2-[5-[(3aS,4S,6aR)- |
| | 2-oxo-1,3,3a,4,6,6a-hexahydrothieno[3,4-d]imidazol-4- |
| | yl]pentanoylamino]ethoxy]ethoxy]ethoxy]ethoxy] |
| | ethoxy]ethoxy]ethylcarbamoyl]phenoxy]pyridine-3- |
| | carbonyl]amino]-5,5-dimethyl-hexanoic acid |
| BF026 | (2S)-2-[[6-[3-[2-[2-[2-[2-[2-[2-[2-[2-[2-[5-[(3aS,4S,6aR)- |
| | 2-oxo-1,3,3a,4,6,6a-hexahydrothieno[3,4-d]imidazol-4- |
| | yl]pentanoylamino]ethoxy]ethoxy]ethoxy]ethoxy]ethoxy] |
| | ethoxy]ethoxy]ethoxy]ethoxy]phenoxy]pyridine- |
| | 3-carbonyl]amino]-5,5-dimethyl-hexanoic acid |
| BF028 | (2S)-2-[[6-[[5-[3-[3-[2-[2-[2-[2-[2-[5-[(3aS,4S,6aR)- |
| | 2-oxo-1,3,3a,4,6,6a-hexahydrothieno[3,4-d]imidazol-4- |
| | yl]pentanoylamino]ethoxy]ethoxy]ethoxy]ethoxy]ethoxy] |
| | propanoylamino]propylcarbamoyl]-1-naphthyl]oxy]pyridine-3- |
| | carbonyl]amino]-5,5-dimethyl-hexanoic acid |

### Sequence Overview

| SEQ | | |
|---|---|---|
| ID | | |
| NO.: | Sequence | Comments |
| 1 | MERPWGAADGLSRWPHGLGLLLLLQLLPPSTLSQDRLDAPP | Sortilin |
| | PPAAPLPRWSGPIGVSWGLRAAAAGGAFPRGGRWRRSAP | |
| | GEDEECGRVRDFVAKLANNTHQHVFDDLRGSVSLSWVGDS | |
| | TGVILVLTTFHVPLVIMTFGQSKLYRSEDYGKNFKDITDLINNT | |
| | FIRTEFGMAIGPENSGKVVLTAEVSGGSRGGRIFRSSDFAKN | |
| | FVQTDLPFHPLTQMMYSPQNSDYLLALSTENGLWVSKNFGG | |
| | KWEEIHKAVCLAKWGSDNTIFFTTYANGSCKADLGALELWR | |
| | TSDLGKSFKTIGVKIYSFGLGGRFLFASVMADKDTTRRIHVST | |
| | DQGDTWSMAQLPSVGQEQFYSILAANDDMVFMHVDEPGDT | |
| | GFGTIFTSDDRGIVYSKSLDRHLYTTTGGETDFTNVTSLRGV | |
| | YITSVLSEDNSIQTMITFDQGGRWTHLRKPENSECDATAKNK | |
| | NECSLHIHASYSISQKLNVPMAPLSEPNAVGIVIAHGSVGDAI | |
| | SVMVPDVYISDDGGYSWTKMLEGPHYYTILDSGGIIVAIEHSS | |
| | RPINVIKFSTDEGQCWQTYTFTRDPIYFTGLASEPGARSMNIS | |
| | IWGFTESFLTSQWVSYTIDFKDILERNCEEKDYTIWLAHSTDP | |
| | EDYEDGCILGYKEQFLRLRKSSMCQNGRDYVVTKQPSICLC | |
| | SLEDFLCDFGYYRPENDSKCVEQPELKGHDLEFCLYGREEH | |
| | LTTNGYRKIPGDKCQGGVNPVREVKDLKKKCTSNFLSPEKQ | |
| | NSKSNSVPIILAIVGLMLVTVVAGVLIVKKYVCGGRFLVHRYS | |
| | VLQQHAEANGVDGVDALDTASHTNKSGYHDDSDEDLLE | |
| 2 | MERPWGAADGLSRWPHGLGLLLLLQLLPPSTLSQDRLDAPP | Sortilin-6His |
| | PPAAPLPRWSGPIGVSWGLRAAAAGGAFPRGGRWRRSAP | |
| | GEDEECGRVRDFVAKLANNTHQHVFDDLRGSVSLSWVGDS | |
| | TGVILVLTTFHVPLVIMTFGQSKLYRSEDYGKNFKDITDLINNT | |
| | FIRTEFGMAIGPENSGKVVLTAEVSGGSRGGRIFRSSDFAKN | |
| | FVQTDLPFHPLTQMMYSPQNSDYLLALSTENGLWVSKNFGG | |
| | KWEEIHKAVCLAKWGSDNTIFFTTYANGSCKADLGALELWR | |
| | TSDLGKSFKTIGVKIYSFGLGGRFLFASVMADKDTTRRIHVST | |
| | DQGDTWSMAQLPSVGQEQFYSILAANDDMVFMHVDEPGDT | |
| | GFGTIFTSDDRGIVYSKSLDRHLYTTTGGETDFTNVTSLRGV | |
| | YITSVLSEDNSIQTMITFDQGGRWTHLRKPENSECDATAKNK | |
| | NECSLHIHASYSISQKLNVPMAPLSEPNAVGIVIAHGSVGDAI | |
| | SVMVPDVYISDDGGYSWTKMLEGPHYYTILDSGGIIVAIEHSS | |
| | RPINVIKFSTDEGQCWQTYTFTRDPIYFTGLASEPGARSMNIS | |
| | IWGFTESFLTSQWVSYTIDFKDILERNCEEKDYTIWLAHSTDP | |
| | EDYEDGCILGYKEQFLRLRKSSMCQNGRDYVVTKQPSICLC | |
| | SLEDFLCDFGYYRPENDSKCVEQPELKGHDLEFCLYGREEH | |
| | LTTNGYRKIPGDKCQGGVNPVREVKDLKKKCTSNFLSPEKQ | |
| | NSKSNSHHHHHH | |
| 3 | MWTLVSWVALTAGLVAGTRCPDGQFCPVACCLDPGGASYS | progranulin |
| | CCRPLLDKWPTTLSRHLGGPCQVDAHCSAGHSCIFTVSGTS | |
| | SCCPFPEAVACGDGHHCCPRGFHCSADGRSCFQRSGNNS | |
| | VGAIQCPDSQFECPDFSTCCVMVDGSWGCCPMPQASCCE | |
| | DRVHCCPHGAFCDLVHTRCITPTGTHPLAKKLPAQRTNRAV | |
| | ALSSSVMCPDARSRCPDGSTCCELPSGKYGCCPMPNATCC | |
| | SDHLHCCPQDTVCDLIQSKCLSKENATTDLLTKLPAHTVGDV | |
| | KCDMEVSCPDGYTCCRLQSGAWGCCPFTQAVCCEDHIHCC | |
| | PAGFTCDTQKGTCEQGPHQVPWMEKAPAHLSLPDPQALKR | |
| | DVPCDNVSSCPSSDTCCQLTSGEWGCCPIPEAVCCSDHQH | |
| | CCPQGYTCVAEGQCQRGSEIVAGLEKMPARRASLSHPRDIG | |
| | CDQHTSCPVGQTCCPSLGGSWACCQLPHAVCCEDRQHCC | |
| | PAGYTCNVKARSCEKEVVSAQPATFLARSPHVGVKDVECGE | |
| | GHFCHDNQTCCRDNRQGWACCPYRQGVCCADRRHCCPA | |
| | GFRCAARGTKCLRREAPRWDAPLRDPALRQLL | |
| 4 | QLYENKPRRPYIL | neurotensin |
| 5 | PYIL | Neurotensin |
| | | 4aa Ct |
| 6 | QLYENK | Neurotensin |
| | | 6aa Nt |
| 7 | IKLSGGVQAKAGVINMDKSESM | |
| 8 | IKLSGGVQAKAGVINMFKSESY | |
| 9 | IKLSGGVQAKAGVINMFKSESYK | |
| 10 | GVQAKAGVINMFKSESY | |
| 11 | GVRAKAGVRNMFKSESY | |
| 12 | GVRAKAGVRN(Nle)FKSESY | |
| 13 | YKSLRRKAPRWDAPLRDPALRQLL | |
| 14 | YKSLRRKAPRWDAYLRDPALRQLL | |
| 15 | YKSLRRKAPRWDAYLRDPALRPLL | |
| 16 | X₅-X₁-X₂-X₃-X₄ | X₅-optional amino acid residue or peptide |
| | | comprising 2 to 30 amino acids or a bond; |
| | | X₁ is R, P, F, Y, L, K, G or H; |
| | | X₂ is Q, Y, L, E or G; |
| | | X₃ is Y, F, L, I, Q, E or N; and |
| | | X₄ is M, K, L or a conservative |
| | | substitution of L |
| 17 | REAPRWDAPLRDPAL | |
| 18 | REALRWDAPLRDPAP | |
| 19 | PYILKRQLYENKPRR | |
| 20 | LYENKPR | |
| 21 | APLRDPAP | |
| 22 | RQLL-OH | |
| 23 | FQLL-OH | |
| 24 | KQLL-OH | |
| 25 | PQLL-OH | |
| 26 | PYIL-OH | |
| 27 | RYLL-OH | |
| 28 | FYLL-OH | |
| 29 | YQLL-OH | |
| 30 | LLQL-OH | |
| 31 | FYIL-OH | |
| 32 | RYIL-OH | |
| 33 | PYLL-OH | |
| 34 | PYYL-OH | |
| 35 | PYFL-OH | |
| 36 | RYYL-OH | |
| 37 | RYFL-OH | |
| 38 | RQFL-OH | |
| 39 | RQYL-OH | |
| 40 | LQLL-OH | |
| 41 | HQLL-OH | |
| 42 | IQLL-OH | |
| 43 | FYYL-OH | |
| 44 | PYMKLAPGELTIIL-OH | |
| 45 | NEKLSQLQTYMI-OH | |
| 46 | KDADLYTSRVMLSSQVP-OH | |
| 47 | RLFKKRRLRSPRVLF-NH2 | amidated C- |
| | | term |
| 48 | ITVDPRLFKKRRLRSPRVLF-NH2 | amidated C- |
| | | term |
| 49 | ITVDPRLFKKRRLRSPRVLFSTQPPR-OH | |
| 50 | WSGPIGVSWGLRAAAAGGAFP-OH | |
| 51 | WSGPIGVSWGLRAAAAGGAFPRGGRWRR-OH, | |
| 52 | GVSWGLR-OH | |
| 53 | REAPRWDAPLRDPALRQLL-OH | |
| 54 | REALRWDAPLRDPAPRQLL-OH | |
| 55 | PYILKRQLYENKPRRPYIL-OH | |
| 56 | LYENKPRRPYIL-OH | |
| 57 | APLRDPAPRQLL-OH | |
| 58 | REAPRWDAPLRDPALFQLL-OH | |
| 59 | REAPRWDAPLRDPALRYLL-OH | |
| 60 | REAPRWDAPLRDPALRQYL-OH | |
| 61 | REAPRWDAPLRDPALRYYL-OH | |
| 62 | TGGFM-OH | |
| 63 | YGGFL-OH | |
| 64 | LYEN-NH3 | amidated C- |
| | | term |
| 65 | LYENK-NH3 | amidatet C- |
| | | term |
| 66 | EVQLVESGGGLVQPGGSLRLSCAASGFTFNNYAMNWVRQA | Alirocumab |
| | PGKGLDWVSTISGSGGTTNYADSVKGRFIISRDSSKHTLYLQ | HC |
| | MNSLRAEDTAVYYCAKDSNWGNFDLWGRGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVNH | |
| | KPSNTKVDKKVEPKSCDKTHTCPPCPAPELLGGPSVFLFPPK | |
| | PKDTLMISRTPEVTCVVVDVSHEDPEVKFNWYVDGVEVHNA | |
| | KTKPREEQYNSTYRVVSVLTVLHQDWLNGKEYKCKVSNKAL | |
| | PAPIEKTISKAKGQPREPQVYTLPPSRDELTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 67 | DIVMTQSPDSLAVSLGERATINCKSSQSVLYRSNNRNFLGW | Alirocumab |
| | YQQKPGQPPNLLIYWASTRESGVPDRFSGSGSGTDFTLTISS | LC |
| | LQAEDVAVYYCQQYYTTPYTFGQGTKLEIKRTVAAPSVFIFP | |
| | PSDEQLKSGTASVVCLLNNFYPREAKVQWKVDNALQSGNS | |
| | QESVTEQDSKDSTYSLSSTLTLSKADYEKHKVYACEVTHQG | |
| | LSSPVTKSFNRGEC | |
| 68 | EVOLVESGGGLVQPGRSLRLSCAASGFTFDDYAMHWVRQA | Adalimumab |
| | PGKGLEWVSAITWNSGHIDYADSVEGRFTISRDNAKNSLYLQ | HC |
| | MNSLRAEDTAVYYCAKVSYLSTASSLDYWGQGTLVTVSSAS | |
| | TKGPSVFPLAPSSKSTSGGTAALGCLVKDYFPEPVTVSWNS | |
| | GALTSGVHTFPAVLQSSGLYSLSSVVTVPSSSLGTQTYICNV | |
| | NHKPSNTKVDKKVEPKSCDKTHTCPPCPAPELLGGPSVFLF | |
| | PPKPKDTLMISRTPEVTCVVVDVSHEDPEVKFNWYVDGVEV | |
| | HNAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSK | |
| | LTVDKSRWQQGNVFSCSVMHEALHNHYTQKSLSLSPGK | |
| 69 | DIQMTQSPSSLSASVGDRVTITCRASQGIRNYLAWYQQKPG | Adalimumab |
| | KAPKLLIYAASTLQSGVPSRFSGSGSGTDFTLTISSLQPEDVA | LC |
| | TYYCQRYNRAPYTFGQGTKVEIKRTVAAPSVFIFPPSDEQLK | |
| | SGTASVVCLLNNFYPREAKVQWKVDNALQSGNSQESVTEQ | |
| | DSKDSTYSLSSTLTLSKADYEKHKVYACEVTHQGLSSPVTKS | |
| | FNRGEC | |
| 70 | MGGTHHHHHHENLYFQGQVQLQESGGGLVQPGGSLRLSC | Nanobody |
| | AASGRTISRYAMSWFRQAPGKEREFVAVARRSGDGAFYAD | (NB) |
| | SVQGRFTVSRDDAKNTVYLQMNSLKPEDTAVYYCAIDSDTF | |
| | YSGSYDYWGQGTQVTVSSE | |
| 71 | MGGTHHHHHHENLYFQGQVQLQESGGGLVQPGGSLRLSC | NB-linker |
| | AASGRTISRYAMSWFRQAPGKEREFVAVARRSGDGAFYAD | |
| | SVQGRFTVSRDDAKNTVYLQMNSLKPEDTAVYYCAIDSDTF | |
| | YSGSYDYWGQGTQVTVSSEGGGGSGGGGSGGGGSGG | |
| 72 | X_{T}X_{L}RQLL-OH | X₁ is T_{L}; X_{L} |
| | | is L_{I} |
| 73 | X_{T}X_{L}FQLL-OH | X_{T} is T_{L}; X_{L} |
| | | is L_{I} |
| 74 | X_{T}X_{L}KQLL-OH | X_{T} is T_{L}; X_{L} |
| | | is L_{I} |
| 75 | X_{T}X_{L}PQLL-OH | X_{T} is T_{L}; X_{L} |
| | | is L_{I} |
| 76 | X_{T}X_{L}PYIL-OH | X_{T} is T_{L}; X_{L} |
| | | is L_{I} |
| 77 | X_{T}X_{L}RYLL-OH | X_{T} is T_{L}; X_{L} |
| | | is L_{I} |
| 78 | X_{T}X_{L}FYLL-OH | X_{T} is T_{L}; X_{L} |
| | | is L_{I} |
| 79 | X_{T}X_{L}YQLL-OH | X_{T} is T_{L}; X_{L} |
| | | is L_{I} |
| 80 | X_{T}X_{L}LLQL-OH | X_{T} is T_{L}; X_{L} |
| | | is L_{I} |
| 81 | X_{T}X_{L}FYIL-OH | X_{T} is T_{L}; X_{L} |
| | | is L_{I} |
| 82 | X_{T}X_{L}RYIL-OH | X_{T} is T_{L}; X_{L} |
| | | is L_{I} |
| 83 | X_{T}X_{L}PYLL-OH | X_{T} is T_{L}; X_{L} |
| | | is L_{I} |
| 84 | X_{T}X_{L}PYYL-OH | X_{T} is T_{L}; X_{L} |
| | | is L_{I} |
| 85 | X_{T}X_{L}PYFL-OH | X_{T} is T_{L}; X_{L} |
| | | is L_{I} |
| 86 | X_{T}X_{L}RYYL-OH | X_{T} is T_{L}; X_{L} |
| | | is L_{I} |
| 87 | X_{T}X_{L}RYFL-OH | X_{T} is T_{L}; X_{L} |
| | | is L_{I} |
| 88 | X_{T}X_{L}RQFL-OH | X_{T} is T_{L}; X_{L} |
| | | is L_{I} |
| 89 | X_{T}X_{L}RQYL-OH | X_{T} is T_{L}; X_{L} |
| | | is L_{I} |
| 90 | X_{T}X_{L}LQLL-OH | X_{T} is T_{L}; X_{L} |
| | | is L_{I} |
| 91 | X_{T}X_{L}HQLL-OH | X_{T} is T_{L}; X_{L} |
| | | is L_{I} |
| 92 | X_{T}X_{L}IQLL-OH | X_{T} IS T_{L}; X_{L} |
| | | is L_{I} |
| 93 | X_{T}X_{L}FYYL-OH | X_{T} is T_{L}; X_{L} |
| | | is L_{I} |
| 94 | X_{T}X_{L}PYMKLAPGELTIIL-OH | X_{T} is T_{L}; X_{L} |
| | | is L_{I} |
| 95 | X_{T}X_{L}NEKLSQLQTYMI-OH | X_{T} is T_{L}; X_{L} |
| | | is L_{I} |
| 96 | X_{T}X_{L}KDADLYTSRVMLSSQVP-OH | X_{T} is T_{L}; X_{L} |
| | | is L_{I} |
| 97 | X_{T}X_{L}RLFKKRRLRSPRVLF-NH2 | X_{T} is T_{L}; X_{L} |
| | | is L_{I} |
| 98 | X_{T}X_{L}ITVDPRLFKKRRLRSPRVLF-NH2 | X_{T} is T_{L}; X_{L} |
| | | is L_{I} |
| 99 | X_{T}X_{L}ITVDPRLFKKRRLRSPRVLFSTQPPR-OH | X_{T} is T_{L}; X_{L} |
| | | is L_{I} |
| 100 | X_{T}X_{L}WSGPIGVSWGLRAAAAGGAFP-OH | X_{T} is T_{L}; X_{L} |
| | | is L_{I} |
| 101 | X_{T}X_{L}WSGPIGVSWGLRAAAAGGAFPRGGRWRR-OH, | X_{T} is T_{L}; X_{L} |
| | | is L |
| 102 | X_{T}X_{L}GVSWGLR-OH | X_{T} is T_{L}; X_{L} |
| | | is L_{I} |
| 103 | X_{T}X_{L}REAPRWDAPLRDPALRQLL-OH | X_{T} is T_{L}; X_{L} |
| | | is L_{I} |
| 104 | X_{T}X_{L}REALRWDAPLRDPAPRQLL-OH | X_{T} is T_{L}; X_{L} |
| | | is L_{I} |
| 105 | X_{T}X_{L}PYILKRQLYENKPRRPYIL-OH | X_{T} is T_{L}; X_{L} |
| | | is L_{I} |
| 106 | X_{T}X_{L}LYENKPRRPYIL-OH | X_{T} is T_{L}; X_{L} |
| | | is L_{I} |
| 107 | X_{T}X_{L}APLRDPAPRQLL-OH | X_{T} is T_{L}; X_{L} |
| | | is L_{I} |
| 108 | X_{T}X_{L}REAPRWDAPLRDPALFQLL-OH | X_{T} is T_{L}; X_{L} |
| | | is L_{I} |
| 109 | X_{T}X_{L}REAPRWDAPLRDPALRYLL-OH | X_{T} is T_{L}; X_{L} |
| | | is L_{I} |
| 110 | X_{T}X_{L}REAPRWDAPLRDPALRQYL-OH | X_{T} is T_{L}; X_{L} |
| | | is L_{I} |
| 111 | X_{T}X_{L}REAPRWDAPLRDPALRYYL-OH | X_{T} is T_{L}; X_{L} |
| | | is L_{I} |
| 112 | X_{T}X_{L}TGGFM-OH | X_{T} is T_{L}; X_{L} |
| | | is L_{I} |
| 113 | X_{T}X_{L}YGGFL-OH | X_{T} is T_{L}; X_{L} |
| | | is L_{I} |
| 114 | X_{T}X_{L}LYEN-NH3 | X_{T} is T_{L}; X_{L} |
| | | is L_{I} |
| 115 | X_{T}X_{L}LYENK-NH3 | X_{T} IS T_{L}; X_{L} |
| | | is L_{I} |
| 116 | Biotin-X_{L}-X₅-X₁X₂X₃X₄ | Biotin attached at N-terminus, XL is L_{I}; |
| | | X₅-optional amino acid residue or peptide |
| | | comprising 2 to 30 amino acids or a bond; |
| | | X₁ is R, P, F, Y, L, K, G or H; |
| | | X₂ is Q, Y, L, E or G; |
| | | X₃ is Y, F, L, I, Q, E or N; and |
| | | X₄ is M, K, L or a conservative |
| | | substitution of L |
| 117 | Biotin-X_{L}-RQLL-OH | Biotin |
| | | attached at |
| | | N-terminus |
| 118 | Biotin-X_{L}-FQLL-OH | Biotin |
| | | attached at |
| | | N-terminus |
| 119 | Biotin-X_{L}-RYLL-OH | Biotin |
| | | attached at |
| | | N-terminus |
| 120 | Biotin-X_{L}-FYLL-OH | Biotin |
| | | attached at |
| | | N-terminus |
| 121 | Biotin-X_{L}-YQLL-OH | Biotin |
| | | attached at |
| | | N-terminus |
| 122 | Biotin-X_{L}-REAPRWDAPLRDPALRQLL-OH | Biotin |
| | | attached at |
| | | N-terminus |
| 123 | Biotin-X_{L}-REALRWDAPLRDPAPRQLL-OH | Biotin |
| | | attached at |
| | | N-terminus |
| 124 | Biotin-X_{L}-REAPRWDAPLRDPALFQLL-OH | Biotin |
| | | attached at |
| | | N-terminus |
| 125 | Biotin-X_{L}-REAPRWDAPLRDPALRYLL-OH | Biotin |
| | | attached at |
| | | N-terminus |
| 126 | Biotin-X_{L}-REAPRWDAPLRDPALRQYL-OH | Biotin |
| | | attached at |
| | | N-terminus |
| 127 | Biotin-X_{L}-REAPRWDAPLRDPALRYYL-OH | Biotin |
| | | attached at |
| | | N-terminus |
| 128 | Biotin-X_{L}-APLRDPAPRQLL-OH | Biotin |
| | | attached at |
| | | N-terminus |
| 129 | Biotin-X_{L}-PYILKRQLYENKPRRPYIL-OH | Biotin |
| | | attached at |
| | | N-terminus |
| 130 | Biotin-X_{L}-LYENKPRRPYIL-OH | Biotin |
| | | attached at |
| | | N-terminus |
| 131 | GGSGGGGGGGGSGG | |
| 132 | GGSGGGG | |
| 133 | GGGGSGGGGSGGGGSGG | |
| 134 | GGSGGGGGGGGS | |
| 135 | GGSGGGGSGGG | |
| 136 | GGSGGGGSG | |
| 137 | GGSGGGG | |
| 138 | GGSGG | |
| 139 | GGS | |
| 140 | CGGSGGGGSGGGGSGG | |
| 141 | Biotin-GGSGGGGSGRQLL-OH | Biotin |
| | | attached at |
| | | N-terminus |
| 142 | Biotin-GGSGGGGSGGGGSRQLL-OH | Biotin |
| | | attached at |
| | | N-terminus |
| 143 | Biotin-GGSGGGGFQLL-OH | Biotin |
| | | attached at |
| | | N-terminus |
| 144 | Biotin-GGSGGFQLL-OH | Biotin |
| | | attached at |
| | | N-terminus |
| 145 | Biotin-GGSGGGGSGFQLL-OH | Biotin |
| | | attached at |
| | | N-terminus |
| 146 | Biotin-GGSGGGGSGGGGSFQLL-OH | Biotin |
| | | attached at |
| | | N-terminus |
| 147 | Biotin-GGSGGGGGGGGSGGRQLL-OH | Biotin |
| | | attached at |
| | | N-terminus |
| 148 | Biotin-GGSGGGGSGGGFQLL-OH | Biotin |
| | | attached at |
| | | N-terminus |
| 149 | Biotin-GGSGGGGSGGGRQLL-OH | Biotin |
| | | attached at |
| | | N-terminus |
| 150 | Biotin-GGSGGGGRQLL-OH | Biotin |
| | | attached at |
| | | N-terminus |
| 151 | Biotin-GGSGGRQLL-OH | Biotin |
| | | attached at |
| | | N-terminus |
| 152 | Biotin-GGSRQLL-OH | Biotin |
| | | attached at |
| | | N-terminus |
| 153 | Biotin-GGRQLL-OH | Biotin |
| | | attached at |
| | | N-terminus |
| 154 | Biotin-RQLL-OH | Biotin |
| | | attached at |
| | | N-terminus |
| 155 | Biotin-GGSGGGGSGGGGSGGFQLL-OH | Biotin |
| | | attached at |
| | | N-terminus |
| 156 | Biotin-GGSGGGGSGGGGSGG-OH | Biotin |
| | | attached at |
| | | N-terminus |
| 157 | Biotin-GGSGGGGSGGGGSGGRQLL-NH2 | |
| 158 | MGGTHHHHHHENLYFQGQVQLQESGGGLVQPGGSLRLSC | NB-linker- |
| | AASGRTISRYAMSWFQAPGKEREFVAVARRSGDGAFYADS | RQLL |
| | VQGRFTVSRDDAKNTVYLQMNSLKPEDTAVYCAIDSDTFYS | |
| | GSYDYWGQGTQVTVSSEGGGGSGGGGSGGGGSGGRQLL | |
| 159 | Alirocumab-CGGSGGGGSGGGGSGGRQLL | Alirocumab- |
| | | link-RQLL |
| 160 | Alirocumab-CGGSGGGGGGGGSGG | Alirocumab- |
| | | link- |
| 161 | Adalimumab-CGGSGGGGSGGGGSGGRQLL | Adalimumab- |
| | | link-RQLL |
| 162 | Adalimumab-CGGSGGGGSGGGGSGG | Adalimumab- |
| | | link |
| 163 | H-RQLI-OH | |
| 164 | H-RQLF-OH | |
| 165 | H-RQLM-OH | |
| 166 | H-RQL-OH | |
| 167 | GGSGGGGSGGGGSGGRQLL-OH | |
| 168 | NHS-PEG4-Mal-CGGSGGGGSGGGGSGGRQLL | |
| 169 | NHS-PEG4-Mal-CGGSGGGGSGGGGSGG | |
| 170 | MGTVSSRRSWWPLPLLLLLLLLLGPAGARAQEDEDGDYEEL | PCSK-9 |
| | VLALRSEEDGLAEAPEHGTTATFHRCAKDPWRLPGTYVVVL | |
| | KEETHLSQSERTARRLQAQAARRGYLTKILHVFHGLLPGFLV | |
| | KMSGDLLELALKLPHVDYIEEDSSVFAQSIPWNLERITPPRYR | |
| | ADEYQPPDGGSLVEVYLLDTSIQSDHREIEGRVMVTDFENVP | |
| | EEDGTRFHRQASKCDSHGTHLAGVVSGRDAGVAKGASMRS | |
| | LRVLNCQGKGTVSGTLIGLEFIRKSQLVQPVGPLVVLLPLAG | |
| | GYSRVLNAACQRLARAGVVLVTAAGNFRDDACLYSPASAPE | |
| | VITVGATNAQDQPVTLGTLGTNFGRCVDLFAPGEDIIGASSD | |
| | CSTCFVSQSGTSQAAAHVAGIAAMMLSAEPELTLAELRQRLI | |
| | HFSAKDVINEAWFPEDQRVLTPNLVAALPPSTHGAGWQLFC | |
| | RTVWSAHSGPTRMATAIARCAPDEELLSCSSFSRSGKRRGE | |
| | RMEAQGGKLVCRAHNAFGGEGVYAIARCCLLPQANCSVHT | |
| | APPAEASMGTRVHCHQQGHVLTGCSSHWEVEDLGTHKPPV | |
| | LRPRGQPNQCVGHREASIHASCCHAPGLECKVKEHGIPAPQ | |
| | EQVTVACEEGWTLTGCSALPGTSHVLGAYAVDNTCVVRSR | |
| | DVSTTGSTSEEAVTAVAICCRSRHLAQASQELQ | |
| 171 | MGTVSSRRSWWPLPLLLLLLLLLGPAGARAQEDEDGDYEEL | PCSK9-6his |
| | VLALRSEEDGLAEAPEHGTTATFHRCAKDPWRLPGTYVVVL | |
| | KEETHLSQSERTARRLQAQAARRGYLTKILHVFHGLLPGFLV | |
| | KMSGDLLELALKLPHVDYIEEDSSVFAQSIPWNLERITPPRYR | |
| | ADEYQPPDGGSLVEVYLLDTSIQSDHREIEGRVMVTDFENVP | |
| | EEDGTRFHRQASKCDSHGTHLAGVVSGRDAGVAKGASMRS | |
| | LRVLNCQGKGTVSGTLIGLEFIRKSQLVQPVGPLVVLLPLAG | |
| | GYSRVLNAACQRLARAGVVLVTAAGNFRDDACLYSPASAPE | |
| | VITVGATNAQDQPVTLGTLGTNFGRCVDLFAPGEDIIGASSD | |
| | CSTCFVSQSGTSQAAAHVAGIAAMMLSAEPELTLAELRQRLI | |
| | HFSAKDVINEAWFPEDQRVLTPNLVAALPPSTHGAGWQLFC | |
| | RTVWSAHSGPTRMATAIARCAPDEELLSCSSFSRSGKRRGE | |
| | RMEAQGGKLVCRAHNAFGGEGVYAIARCCLLPQANCSVHT | |
| | APPAEASMGTRVHCHQQGHVLTGCSSHWEVEDLGTHKPPV | |
| | LRPRGQPNQCVGHREASIHASCCHAPGLECKVKEHGIPAPQ | |
| | EQVTVACEEGWTLTGCSALPGTSHVLGAYAVDNTCVVRSR | |
| | DVSTTGSTSEEAVTAVAICCRSRHLAQASQELQHHHHHH | |
| 172 | Ac-TVFTSWEEYLDWVGGSGGGGSGGGGSGGRQLL-OH | pep-010 |
| 173 | Plasmid for Nanobody-link-RQLL | |
| 174 | Plasmid for NB-link | |
| 175 | CGGSGGGGSGGGGSGGRQLL | ApoB |
| 176 | MDPPRPALLALLALPALLLLLLAGARAEEEMLENVSLVCPKDA | |
| | TRFKHLRKYTYNYEAESSSGVPGTADSRSATRINCKVELEVP | |
| | QLCSFILKTSQCTLKEVYGFNPEGKALLKKTKNSEEFAAAMS | |
| | RYELKLAIPEGKQVFLYPEKDEPTYILNIKRGIISALLVPPETEE | |
| | AKQVLFLDTVYGNCSTHFTVKTRKGNVATEISTERDLGQCDR | |
| | FKPIRTGISPLALIKGMTRPLSTLISSSQSCQYTLDAKRKHVAE | |
| | AICKEQHLFLPFSYKNKYGMVAQVTQTLKLEDTPKINSRFFG | |
| | EGTKKMGLAFESTKSTSPPKQAEAVLKTLQELKKLTISEQNIQ | |
| | RANLFNKLVTELRGLSDEAVTSLLPQLIEVSSPITLQALVQCG | |
| | QPQCSTHILQWLKRVHANPLLIDVVTYLVALIPEPSAQQLREI | |
| | FNMARDQRSRATLYALSHAVNNYHKTNPTGTQELLDIANYL | |
| | MEQIQDDCTGDEDYTYLILRVIGNMGQTMEQLTPELKSSILK | |
| | CVQSTKPSLMIQKAAIQALRKMEPKDKDQEVLLQTFLDDASP | |
| | GDKRLAAYLMLMRSPSQADINKIVQILPWEQNEQVKNFVASH | |
| | IANILNSEELDIQDLKKLVKEALKESQLPTVMDFRKFSRNYQL | |
| | YKSVSLPSLDPASAKIEGNLIFDPNNYLPKESMLKTTLTAFGF | |
| | ASADLIEIGLEGKGFEPTLEALFGKQGFFPDSVNKALYWVNG | |
| | QVPDGVSKVLVDHFGYTKDDKHEQDMVNGIMLSVEKLIKDL | |
| | KSKEVPEARAYLRILGEELGFASLHDLQLLGKLLLMGARTLQ | |
| | GIPQMIGEVIRKGSKNDFFLHYIFMENAFELPTGAGLQLQISS | |
| | SGVIAPGAKAGVKLEVANMQAELVAKPSVSVEFVTNMGIIIPD | |
| | FARSGVQMNTNFFHESGLEAHVALKAGKLKFIIPSPKRPVKLL | |
| | SGGNTLHLVSTTKTEVIPPLIENRQSWSVCKQVFPGLNYCTS | |
| | GAYSNASSTDSASYYPLTGDTRLELELRPTGEIEQYSVSATY | |
| | ELQREDRALVDTLKFVTQAEGAKQTEATMTFKYNRQSMTLS | |
| | SEVQIPDFDVDLGTILRVNDESTEGKTSYRLTLDIQNKKITEVA | |
| | LMGHLSCDTKEERKIKGVISIPRLQAEARSEILAHWSPAKLLL | |
| | QMDSSATAYGSTVSKRVAWHYDEEKIEFEWNTGTNVDTKK | |
| | MTSNFPVDLSDYPKSLHMYANRLLDHRVPQTDMTFRHVGSK | |
| | LIVAMSSWLQKASGSLPYTQTLQDHLNSLKEFNLQNMGLPD | |
| | FHIPENLFLKSDGRVKYTLNKNSLKIEIPLPFGGKSSRDLKML | |
| | ETVRTPALHFKSVGFHLPSREFQVPTFTIPKLYQLQVPLLGVL | |
| | DLSTNVYSNLYNWSASYSGGNTSTDHFSLRARYHMKADSV | |
| | VDLLSYNVQGSGETTYDHKNTFTLSYDGSLRHKFLDSNIKFS | |
| | HVEKLGNNPVSKGLLIFDASSSWGPQMSASVHLDSKKKQHL | |
| | FVKEVKIDGQFRVSSFYAKGTYGLSCQRDPNTGRLNGESNL | |
| | RFNSSYLQGTNQITGRYEDGTLSLTSTSDLQSGIIKNTASLKY | |
| | ENYELTLKSDTNGKYKNFATSNKMDMTFSKQNALLRSEYQA | |
| | DYESLRFFSLLSGSLNSHGLELNADILGTDKINSGAHKATLRI | |
| | GQDGISTSATTNLKCSLLVLENELNAELGLSGASMKLTTNGR | |
| | FREHNAKFSLDGKAALTELSLGSAYQAMILGVDSKNIFNFKV | |
| | SQEGLKLSNDMMGSYAEMKFDHTNSLNIAGLSLDFSSKLDNI | |
| | YSSDKFYKQTVNLQLQPYSLVTTLNSDLKYNALDLTNNGKLR | |
| | LEPLKLHVAGNLKGAYQNNEIKHIYAISSAALSASYKADTVAK | |
| | VQGVEFSHRLNTDIAGLASAIDMSTNYNSDSLHFSNVFRSVM | |
| | APFTMTIDAHTNGNGKLALWGEHTGQLYSKFLLKAEPLAFTF | |
| | SHDYKGSTSHHLVSRKSISAALEHKVSALLTPAEQTGTWKLK | |
| | TQFNNNEYSQDLDAYNTKDKIGVELTGRTLADLTLLDSPIKVP | |
| | LLLSEPINIIDALEMRDAVEKPQEFTIVAFVKYDKNQDVHSINL | |
| | PFFETLQEYFERNRQTIIVVLENVQRNLKHINIDQFVRKYRAA | |
| | LGKLPQQANDYLNSFNWERQVSHAKEKLTALTKKYRITENDI | |
| | QIALDDAKINFNEKLSQLQTYMIQFDQYIKDSYDLHDLKIAIANI | |
| | IDEIIEKLKSLDEHYHIRVNLVKTIHDLHLFIENIDFNKSGSSTAS | |
| | WIQNVDTKYQIRIQIQEKLQQLKRHIQNIDIQHLAGKLKQHIEAI | |
| | DVRVLLDQLGTTISFERINDILEHVKHFVINLIGDFEVAEKINAF | |
| | RAKVHELIERYEVDQQIQVLMDKLVELAHQYKLKETIQKLSNV | |
| | LQQVKIKDYFEKLVGFIDDAVKKLNELSFKTFIEDVNKFLDMLI | |
| | KKLKSFDYHQFVDETNDKIREVTQRLNGEIQALELPQKAEAL | |
| | KLFLEETKATVAVYLESLQDTKITLIINWLQEALSSASLAHMKA | |
| | KFRETLEDTRDRMYQMDIQQELQRYLSLVGQVYSTLVTYISD | |
| | WWTLAAKNLTDFAEQYSIQDWAKRMKALVEQGFTVPEIKTIL | |
| | GTMPAFEVSLQALQKATFQTPDFIVPLTDLRIPSVQINFKDLK | |
| | NIKIPSRFSTPEFTILNTFHIPSFTIDFVEMKVKIIRTIDQMLNSE | |
| | LOWPVPDIYLRDLKVEDIPLARITLPDFRLPEIAIPEFIIPTLNLN | |
| | DFQVPDLHIPEFQLPHISHTIEVPTFGKLYSILKIQSPLFTLDAN | |
| | ADIGNGTTSANEAGIAASITAKGESKLEVLNFDFQANAQLSNP | |
| | KINPLALKESVKFSSKYLRTEHGSEMLFFGNAIEGKSNTVASL | |
| | HTEKNTLELSNGVIVKINNQLTLDSNTKYFHKLNIPKLDFSSQ | |
| | ADLRNEIKTLLKAGHIAWTSSGKGSWKWACPRFSDEGTHES | |
| | QISFTIEGPLTSFGLSNKINSKHLRVNQNLVYESGSLNFSKLEI | |
| | QSQVDSQHVGHSVLTAKGMALFGEGKAEFTGRHDAHLNGK | |
| | VIGTLKNSLFFSAQPFEITASTNNEGNLKVRFPLRLTGKIDFLN | |
| | NYALFLSPSAQQASWQVSARFNQYKYNQNFSAGNNENIME | |
| | AHVGINGEANLDFLNIPLTIPEMRLPYTIITTPPLKDFSLWEKT | |
| | GLKEFLKTTKQSFDLSVKAQYKKNKHRHSITNPLAVLCEFISQ | |
| | SIKSFDRHFEKNRNNALDFVTKSYNETKIKFDKYKAEKSHDEL | |
| | PRTFQIPGYTVPVVNVEVSPFTIEMSAFGYVFPKAVSMPSFSI | |
| | LGSDVRVPSYTLILPSLELPVLHVPRNLKLSLPDFKELCTISHI | |
| | FIPAMGNITYDFSFKSSVITLNTNAELFNQSDIVAHLLSSSSSVI | |
| | DALQYKLEGTTRLTRKRGLKLATALSLSNKFVEGSHNSTVSL | |
| | TTKNMEVSVATTTKAQIPILRMNFKQELNGNTKSKPTVSSSM | |
| | EFKYDFNSSMLYSTAKGAVDHKLSLESLTSYFSIESSTKGDV | |
| | KGSVLSREYSGTIASEANTYLNSKSTRSSVKLQGTSKIDDIW | |
| | NLEVKENFAGEATLQRIYSLWEHSTKNHLQLEGLFFTNGEHT | |
| | SKATLELSPWQMSALVQVHASQPSSFHDFPDLGQEVALNAN | |
| | TKNQKIRWKNEVRIHSGSFQSQVELSNDQEKAHLDIAGSLEG | |
| | HLRFLKNIILPVYDKSLWDFLKLDVTTSIGRRQHLRVSTAFVY | |
| | TKNPNGYSFSIPVKVLADKFIIPGLKLNDLNSVLVMPTFHVPFT | |
| | DLQVPSCKLDFREIQIYKKLRTSSFALNLPTLPEVKFPEVDVL | |
| | TKYSQPEDSLIPFFEITVPESQLTVSQFTLPKSVSDGIAALDLN | |
| | AVANKIADFELPTIIVPEQTIEIPSIKFSVPAGIVIPSFQALTARF | |
| | EVDSPVYNATWSASLKNKADYVETVLDSTCSSTVQFLEYEL | |
| | NVLGTHKIEDGTLASKTKGTFAHRDFSAEYEEDGKYEGLQE | |
| | WEGKAHLNIKSPAFTDLHLRYQKDKKGISTSAASPAVGTVGM | |
| | DMDEDDDFSKWNFYYSPQSSPDKKLTIFKTELRVRESDEET | |
| | QIKVNWEEEAASGLLTSLKDNVPKATGVLYDYVNKYHWEHT | |
| | GLTLREVSSKLRRNLQNNAEWVYQGAIRQIDDIDVRFQKAAS | |
| | GTTGTYQEWKDKAQNLYQELLTQEGQASFQGLKDNVFDGL | |
| | VRVTQEFHMKVKHLIDSLIDFLNFPRFQFPGKPGIYTREELCT | |
| | MFIREVGTVLSQVYSKVHNGSEILFSYFQDLVITLPFELRKHK | |
| | LIDVISMYRELLKDLSKEAQEVFKAIQSLKTTEVLRNLQDLLQF | |
| | IFQLIEDNIKQLKEMKFTYLINYIQDEINTIFSDYIPYVFKLLKEN | |
| | LCLNLHKFNEFIQNELQEASQELQQIHQYIMALREEYFDPSIV | |
| | GWTVKYYELEEKIVSLIKNLLVALKDFHSEYIVSASNFTSQLSS | |
| | QVEQFLHRNIQEYLSILTDPDGKGKEKIAELSATAQEIIKSQAI | |
| | ATKKIISDYHQQFRYKLQDFSDQLSDYYEKFIAESKRLIDLSIQ | |
| | NYHTFLIYITELLKKLQSTTVMNPYMKLAPGELTIIL | |
| 177 | MTILFLTMVISYFGCMKAAPMKEANIRGQGGLAYPGVRTHGT | BDNF |
| | LESVNGPKAGSRGLTSLADTFEHVIEELLDEDQKVRPNEENN | |
| | KDADLYTSRVMLSSQVPLEPPLLFLLEEYKNYLDAANMSMRV | |
| | RRHSDPARRGELSVCDSISEWVTAADKKTAVDMSGGTVTVL | |
| | EKVPVSKGQLKQYFYETKCNPMGYTKEGCRGIDKRHWNSQ | |
| | CRTTQSYVRALTMDSKKRIGWRFIRIDTSCVCTLTIKRGR | |
| 178 | MSMLFYTLITAFLIGIQAEPHSESNVPAGHTIPQAHWTKLQHS | NGF |
| | LDTALRRARSAPAAAIAARVAGQTRNITVDPRLFKKRRLRSP | |
| | RVLFSTQPPREAADTQDLDFEVGGAAPFNRTHRSKRSSSHP | |
| | IFHRGEFSVCDSVSVWVGDKTTATDIKGKEVMVLGEVNINNS | |
| | VFKQYFFETKCRDPNPVDSGCRGIDSKHWNSYCTTTHTFVK | |
| | ALTMDGKQAAWRFIRIDTACVCVLSRKAVRRA | |
| 179 | WSGPIGVSWGLRAAAAGFQLL-OH | |
| 180 | WSGPIGVSWGLRAAAAG | |
| 181 | CREAPRWDAPLRDPAL | |
| 182 | WSGPIGVSWGLRAAAAGFQLL-OH | |
| 183 | WSGPIGVSWGLRAAAAGRQLL-OH | |
| 184 | CREAPRWDAPLRDPALRQLL-OH | |
| 185 | CREAPRWDAPLRDPALROLL-NH2 | |
| 186 | CREAPRWDAPLRDPALFQLL-OH | |
| 187 | CREAPRWDAPLRDPALFQLL-NH2 | |
| 188 | Biotin-REAPRWDAPLRDPALFQLL-NH2 | |
| 189 | Biotin-REAPRWDAPLRDPALFQLL-OH | |
| 190 | Biotin-REAPRWDAPLRDPALRQLL-NH2 | |
| 191 | Biotin-REAPRWDAPLRDPALRQLL-OH | |
| 192 | CREAPRWDAPLRDPALRQLL | Conjugated |
| | | to |
| | | adalimubab |
| 193 | CREAPRWDAPLRDP ALRQLL-NH2 | Conjugated |
| | | to |
| | | adalimumab |
| 194 | CREAPRWDAPLRDP ALFQLL-OH | Conjugated |
| | | to |
| | | adalimumab |
| 195 | CREAPRWDAPLRDPALFQLL-NH2 | Conjugated |
| | | to |
| | | adalimumab |
| 196 | AARL-OH | |
| 197 | PIPLV-OH | |

### REFERENCES

1. Sun, X., et al., PROTACs: great opportunities for academia and industry. Signal Transduct Target Ther, 2019. 4: p. 64.
2. Banik, S. M., et al., Lysosome-targeting chimaeras for degradation of extracellular proteins. Nature, 2020. 584 (7820): p. 291-297.
3. Braulke, T. and J.S. Bonifacino, Sorting of lysosomal proteins. Biochim Biophys Acta, 2009. 1793 (4): p. 605-14.
4. Lefrancois, S., et al., The lysosomal trafficking of sphingolipid activator proteins (SAPs) is mediated by sortilin. EMBO J, 2003. 22 (24): p. 6430-7.
5. Ni, X. and C.R. Morales, The lysosomal trafficking of acid sphingomyelinase is mediated by sortilin and mannose 6-phosphate receptor. Traffic, 2006. 7 (7): p. 889-902.
6. Hu, F., et al., Sortilin-mediated endocytosis determines levels of the frontotemporal dementia protein, progranulin. Neuron, 2010. 68 (4): p. 654-67.
7. Lee, W. C., et al., Targeted manipulation of the sortilin-progranulin axis rescues progranulin haploinsufficiency. Hum Mol Genet, 2014. 23 (6): p. 1467-78.
8. Schroder, T. J., et al., The identification of AF38469: an orally bioavailable inhibitor of the VPS10P family sorting receptor Sortilin. Bioorg Med Chem Lett, 2014. 24 (1): p. 177-80.
9. Andersen, J. L., et al., The identification of novel acid isostere based inhibitors of the VPS10P family sorting receptor Sortilin. Bioorg Med Chem Lett, 2017. 27 (11): p. 2629-2633.
10. Stachel, S. J., et al., Identification of potent inhibitors of the sortilin-progranulin interaction. Bioorg Med Chem Lett, 2020. 30 (17): p. 127403.
11. Quistgaard, E. M., et al., Revisiting the structure of the Vps10 domain of human sortilin and its interaction with neurotensin. Protein Sci, 2014. 23 (9): p. 1291-300.
12. Leloup, N., et al., Low pH-induced conformational change and dimerization of sortilin triggers endocytosed ligand release. Nat Commun, 2017. 8 (1): p. 1708.
13. Petersen CM, Nielsen MS, Nykjaer A, Jacobsen L, Tommerup N, Rasmussen HH, Roigaard H, Gliemann J, Madsen P, Moestrup SK. Molecular identification of a novel candidate sorting receptor purified from human brain by receptor-associated protein affinity chromatography. J. Biol. Chem. 1997 Feb. 7; 272 (6): 3599-605.
14. Andersen J L, Schrøder T J, Christensen S, Strandbygård D, Pallesen L T, García-Alai M M, Lindberg S, Langgård M, Eskildsen J C, David L, Tagmose L, Simonsen K B, Maltas P J, Rønn L C, de Jong I E, Malik I J, Egebjerg J, Karlsson J J, Uppalanchi S, Sakumudi D R, Eradi P, Watson S P, Thirup S. Identification of the first small-molecule ligand of the neuronal receptor sortilin and structure determination of the receptor-ligand complex. Acta Crystallogr D Biol Crystallogr. 2014 February; 70 (Pt 2): 451-60. doi: 10.1107/S1399004713030149. Epub 2014 Jan. 29. PMID: 24531479; PMCID: PMC3940197.
15. Robert P. Sparks, Andres S. Arango, Jermaine L. Jenkins, Wayne C. Guida, Emad Tajkhorshid, Charles E. Sparks, Janet D. Sparks, and Rutilio A. Fratti Biochemistry 2020 59 (45), 4321-4335
WO 2014/114779
US 2016/0331646
WO 2017/009327
WO 2019/016247
WO 2021/263279
WO 2021/116290
WO 2020/252066
WO 2020/014617
US 2017/0218058
WO 2016/164637
WO 2020/037434

1. A bifunctional compound comprising the structure of Formula (I): 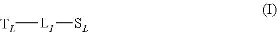
wherein
S_{L} is a moiety that binds to Sortilin;
L_{I} is a linker or a bond; and
T_{L} is a moiety that binds an extracellular target molecule;
or a pharmaceutically acceptable salt thereof.

2. The bifunctional compound according to claim 1, wherein S_{L} has a structure according to Formula III: 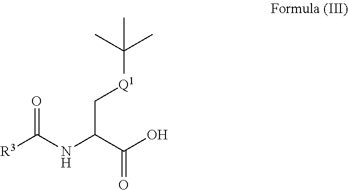
wherein Q¹ is a bond or —CH₂—; and
R³ is of Formula (IIIa), Formula (IIIb) or Formula B; 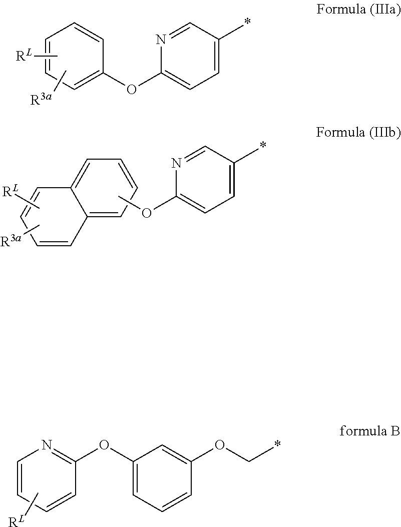
wherein, R^{3a} is selected from the group consisting of H, halogen, alkoxy, —CF₃, and an optionally substituted C₁-C₄ alkyl; and
wherein * denotes the attachment with Formula (III) and R^{L} denotes the attachment with L_{I}.

3. The bifunctional compound according to any one of the preceding claims, wherein S_{L} is according to any one selected from the group consisting of formulas IIIc, IIId, IIIe, IIIf, IIIg and IIIh: 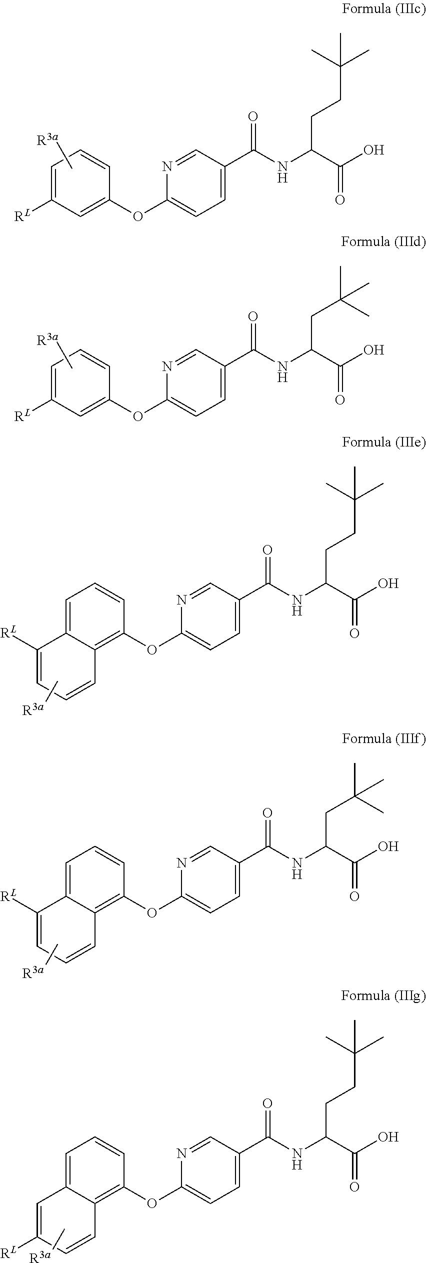 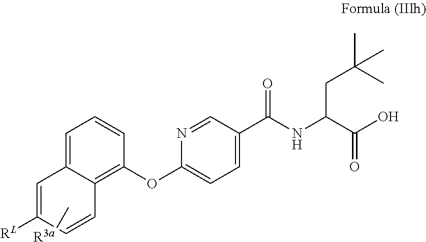
wherein R^{3a} is selected from the group consisting of H, halogen, alkoxy, —CF₃, and an optionally substituted C₁-₄ alkyl; and
R^{L} denotes the attachment with L_{I}.

4. The bifunctional compound according to any one of claims 2 to 3, wherein R^{3a} is H.

5. The bifunctional compound according to any one of claims 2 to 3, wherein R^{3a} is halogen or —CF₃.

6. The bifunctional compound according to any one of claims 2 to 3, wherein R^{3a} is alkoxy.

7. The bifunctional compound according to any one of claims 2 to 3, wherein R^{3a} is an optionally substituted C₁-₄ alkyl.

8. The bifunctional compound according to any one of claims 2 to 3, wherein S_{L} is selected from the group consisting of: 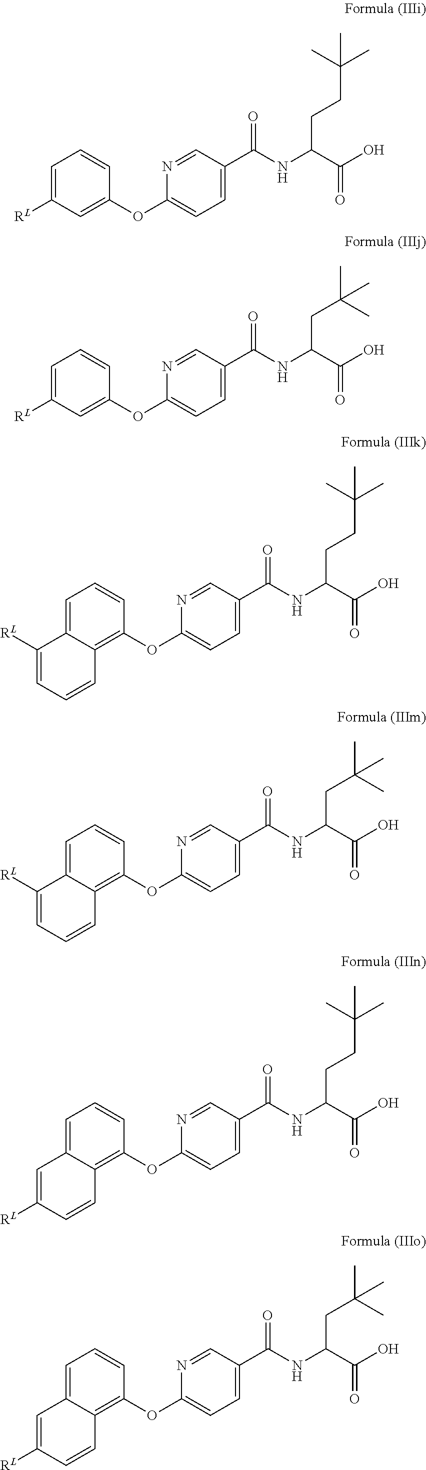
wherein R^{L} denotes the attachment with L_{I}.

9. The bifunctional compound according to any one of claims 2 to 3 and 8, wherein S_{L} is according to formula (IIIi): 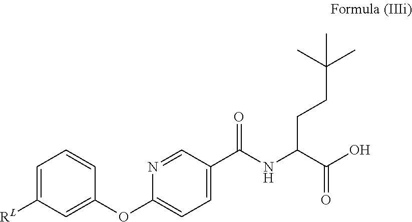
wherein R^{L} denotes the attachment with L_{I}.

10. The bifunctional compound according to any one of claims 2 to 3 and 8, wherein S_{L} is according to formula (IIIj): 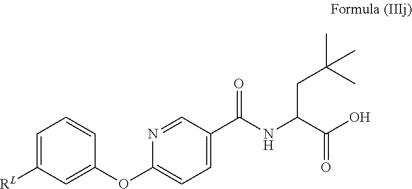
wherein R^{L} denotes the attachment with L_{I}.

11. The bifunctional compound according to any one of claims 2 to 3 and 8, wherein S_{L} is according to formula (IIIk): 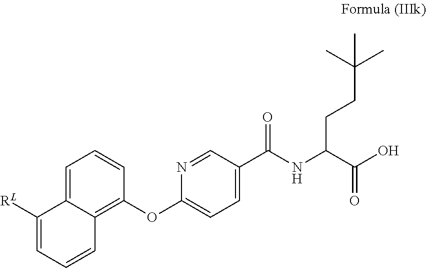
wherein R^{L} denotes the attachment with L_{I}.

12. The bifunctional compound according to any one of claims 2 to 3 and 8, wherein S_{L} is according to formula (IIIm): 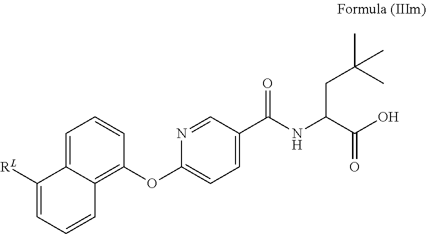
wherein R^{L} denotes the attachment with L_{I}.

13. The bifunctional compound according to any one of claims 2 to 3 and 8, wherein S_{L} is according to formula (IIIn): 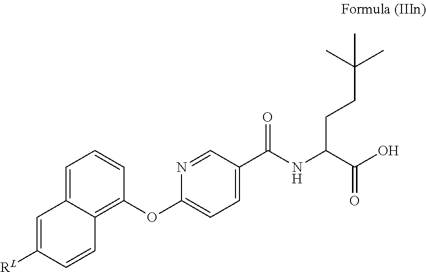
wherein R^{L} denotes the attachment with L_{I}.

14. The bifunctional compound according to any one of claims 2 to 3 and 8, wherein S_{L} is according to formula (IIIn): 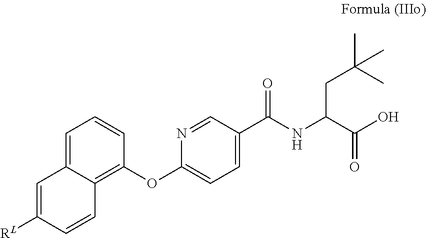
wherein R^{L} denotes the attachment with L_{I}.

15. The bifunctional compound according to any one of the preceding claims, wherein S_{L} is the S-stereoisomer of any one of formulas III, or formulas IIIa to IIIo.

16. The bifunctional compound according to any one of the preceding claims, wherein S_{L} is the R-stereoisomer of any one of formulas III, or formulas IIIa to IIIo.

17. The bifunctional compound according any one of claims 1 and 2, wherein S_{L} is a according to formula (D-I): 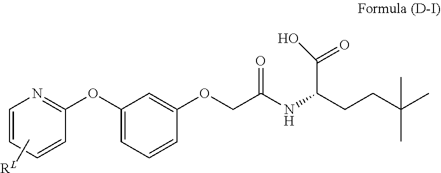
wherein R^{L} denotes the attachment to L_{I}.

18. The bifunctional compound according any one of claims 1, 2 and 17, wherein S_{L} is according to formula (D-II): 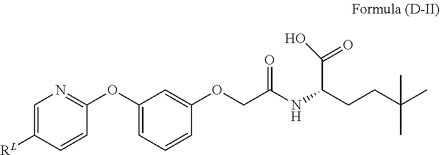
wherein R^{L} denotes attachment to L_{I}.

19. The bifunctional compound according to claim 1, wherein S_{L} has a structure according to formula (II): 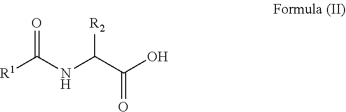
wherein
R¹ is selected from the group consisting of heteroaryl, aryl, heterocycle, C₃-C₁₀ cycloalkyl, —(C₁-C₅ alkyl)-aryl and C₁-C₁₀ alkyl, —B—O-aryl, each of which is optionally substituted with one or more, identical or different, substituents R^{IIa}; wherein B is an optionally substituted C₁-C₅ alkyl;
R² is selected from the group consisting of C₁-C₁₀ alkyl and C₂-C₁₀ alkenyl, each of which is optionally substituted with one or more, identical or different, substituents R^{IIb};
R^{IIa} is selected from the group consisting of —O-aryl, —CH₂-aryl, Cl, Br, F, C₁-C₃ alkoxy and C₁-C₃ alkyl, wherein each of —O-aryl, —CH₂-aryl, C₁-C₃ alkoxy and C₁-C₃ alkyl is optionally substituted with one or more, identical or different substituents selected from the group consisting of H, —O—CH₂—C(═O)O—, —O—CH₂—C(═O)NH, halogen, alkoxy, —CF₃, and an optionally substituted C₁-C₄ alkyl;
R^{IIb} is selected from the group consisting of C₁-C₁₀ alkoxy, C₃-C₁₀ cycloalkyl, C₁-C₁₀ alkyl and —CH₂-aryl;
wherein S_{L} is conjugated to L_{I} via R¹ or R^{IIa}.

20. The bifunctional compound according to claim 19, wherein R¹ is heteroaryl, optionally substituted with one or more, identical or different, substituents R^{IIa}.

21. The bifunctional compound according to any one of claim 19 or 20, wherein R¹ is pyridyl, optionally substituted with one or more, identical or different, substituents R^{IIa}.

22. The bifunctional compound according to any one of claims 19 to 21, wherein R^{IIa} is —O-aryl optionally substituted with one or more, identical or different substituents selected from the group consisting of H, halogen, alkoxy, —CF₃, and an optionally substituted C₁-C₄ alkyl.

23. The bifunctional compound according to any one of claims 19 to 22, wherein R^{IIa} is —O-phenyl or —O-naphthyl optionally substituted with one or more, identical or different substituents selected from the group consisting of H, halogen, alkoxy, —CF₃, and an optionally substituted C₁-C₄ alkyl.

24. The bifunctional compound according to any one of claims 19 to 23, wherein R¹ is of Formula (IIIa) or Formula (IIIb): 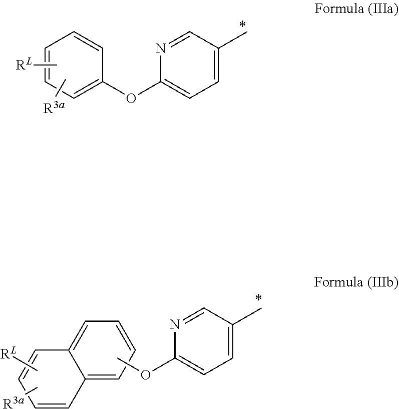
wherein
R^{3a} is selected from the group consisting of H, halogen, alkoxy, —CF₃, and an optionally substituted C₁-C₄ alkyl;
wherein * denotes the attachment with Formula (II) and R^{L} denotes the attachment with L_{I}.

25. The compound according to claim 19, wherein R¹ is according to formula B: 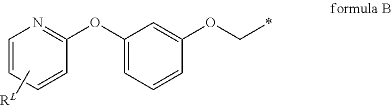
wherein R^{L} denotes the attachment to L_{I} and * denotes the attachment to formula II.

26. The bifunctional compound according to any one of claims 19 to 25, wherein R² is C₁-C₁₀ alkyl, such as C₁, C₂, C₃, C₄, C₅, C₆, C₇, C₈, C₉ or C₁₀ alkyl, optionally substituted with one or more, identical or different, substituents RIlb.

27. The bifunctional compound according to any one of claims 19 to 26, wherein R² is of Formula (IIa): 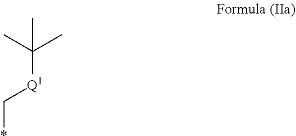
wherein Q¹ is a bond or —CH₂—; and
wherein * denotes the attachment with Formula (II).

28. The bifunctional compound according to claim 1, S_{L} has a structure according to formula IV: 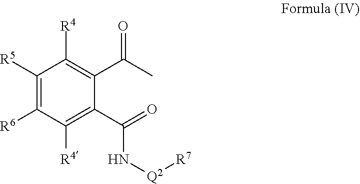
wherein
R⁴ is H or F;
R^{4′} is H;
R⁵ is halogen, H, C₁-C₆ alkyl, C₂-₆ alkenyl or C₁-C₆ haloalkyl;
R⁶ is halogen, H, C₁-C₆ alkyl or C₁-C₆ haloalkyl;
Q² is a bond or CH₂;
R⁷ is a 5-6 membered heteroaromatic monocyclic ring with one or two heteroatom(s), wherein the heteroaromatic ring is optionally substituted with one or two substituents individually selected from the group consisting of —CN; C₁-C₃ alkyl; halogenated C₁-C₃ alkyl; C₁-C₃ alkoxy, halogen; —C(O)NH—C₁-C₃ alkyl; aryl optionally substituted with —C(O)NH—C₁-C₃ alkyl; and optionally substituted heteroaryl,
or a pharmaceutically acceptable salt thereof, wherein S_{L} is conjugated to L_{I} via R⁷.

29. The bifunctional compound according to claim 28, wherein R⁴ is H.

30. The bifunctional compound according to any one of claim 28 or 29, wherein R^{4′} is H.

31. The bifunctional compound according to any one of claims 28 to 30, wherein R⁵ is halogen, H, or C₁-C₆ haloalkyl.

32. The bifunctional compound according to any one of claims 28 to 31, wherein R⁶ is halogen, H, or C₁-C₆ haloalkyl.

33.

34. The bifunctional compound according to any one of claims 28 to 32, wherein R⁵ is halogen, H, or C₁-C₆ haloalkyl; and R⁶ is halogen, H, C₁-C₆ alkyl or C₁-C₆ haloalkyl.

35. The bifunctional compound according to any one of claims 28 to 34, wherein R⁵ is H or —CF₃.

36. The bifunctional compound according to any one of claims 28 to 35, wherein R⁶ is H or —CF₃.

37. The bifunctional compound according to claims 28 to 36, wherein at least one of R⁵ and R⁶ is —CF₃.

38. The bifunctional compound according to claims 28 to 36, wherein one of R⁵ or R⁶ is —CF₃, and one of R⁵ or R⁶ is H.

39. The bifunctional compound according to any one of claims 28 to 38, wherein
R⁴ is H,
R⁵ is —CF₃ or H;
R⁶ is —CF₃ or H; and
R^{4′} is H.

40. The bifunctional compound according to any one of claims 28 to 39, wherein Q² is a bond.

41. The bifunctional compound according to any one of claims 28 to 40, wherein R⁷ is a 6-membered heteroaromatic monocyclic ring with one heteroatom substituted with a C₁ alkyl, wherein said heteroaromatic ring is optionally further substituted with an optionally substituted aryl,
and wherein S_{L} is conjugated to L_{I} via R⁷.

42. The bifunctional compound according to claims 28 to 41, wherein Q² is a bond,
R⁴ is H,
R⁵ is —CF₃ or H;
R⁶ is —CF₃ or H;
R^{4′} is H,
Q² is a bond
R⁷ is a 6-membered heteroaromatic monocyclic ring with one heteroatom substituted with a C₁ alkyl, wherein said heteroaromatic ring is optionally further substituted with an optionally substituted aryl,
and wherein S_{L} is conjugated to L_{I} via R⁷.

43. The bifunctional compound according to any one of claims 28 to 42, wherein R₇ is a pyridyl substituted with a C₁ alkyl, wherein said pyridyl is optionally further substituted with an optionally substituted aryl.

44. The bifunctional compound according to any one of claims 28 to 43, wherein R₇ is a pyridyl substituted with a C₁ alkyl.

45. The bifunctional compound according to any one of claims 28 to 45, wherein R₇ is a pyridyl substituted with C₁ alkyl and phenyl.

46. The bifunctional compound according to any one of claims 28 to 45, wherein R₇ has a structure according to formula (IVa) or Formula (IVb): 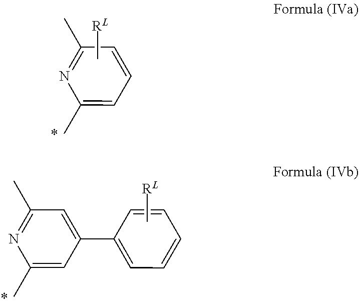
wherein * denotes attachment to formula (IV) and wherein R^{L} denotes the attachment to L_{I}.

47. The bifunctional compound according to claim any one of claims 28 to 46, wherein the S_{L} is selected from any one of the group consisting of formulas IVc, IVd, IVe and IVf: 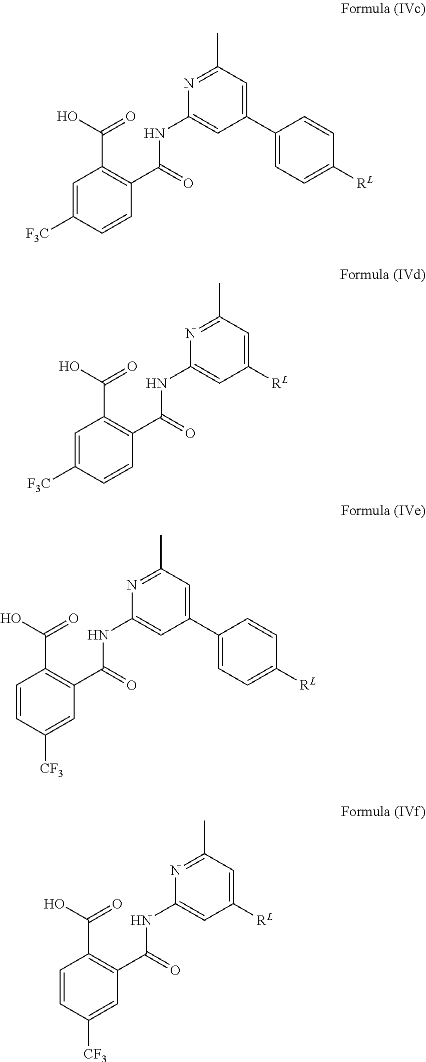
wherein R^{L} denotes the attachment to L_{I}.

48. The bifunctional compound according to claim 1, wherein S_{L} is a substituent derived from a compound of formula VI: 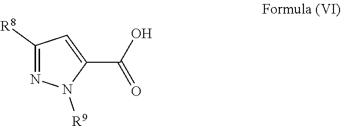
wherein
R⁸ is C₁-C₁₀ alkyl; and
R⁹ is selected from the group consisting of C₁-C₁₀ alkyl, aryl, and heteroaryl, each of which is optionally substituted with one or more, identical or different, substituents R^{VIa};
R^{VIa} is selected from the group consisting of aryl, heteroaryl, —O-aryl, —O-heteroaryl and halogen, wherein each of aryl, heteroaryl, —O-aryl and —O-heteroaryl is optionally substituted with one or more halogen(s).

49. The bifunctional compound according to claim 1, wherein S_{L} is a substituent derived from a compound of formula (VI): 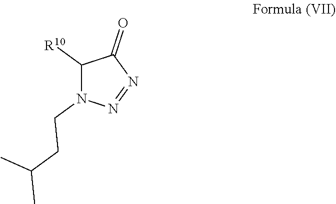
wherein
R¹⁰ is 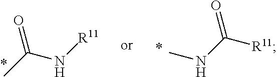
and
R¹¹ is an optionally substituted heteroaryl or an optionally substituted aryl.

50. The bifunctional compound according to claim 1, wherein S_{L} is a substituent derived from a compound selected form the group consisting of:

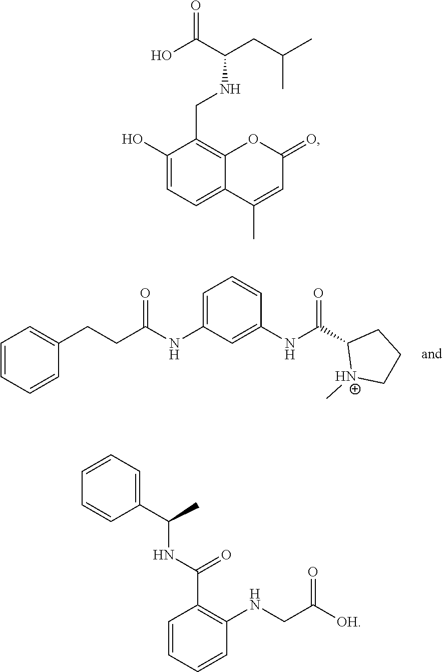 [SMILES: Cc1cc(=O)oc2c(CN[C@@H](CC(C)C)C(=O)O)c(O)ccc12 ; C[NH+]1CCC[C@H]1C(=O)Nc1cccc(NC(=O)CCc2ccccc2)c1 ; C[C@@H](NC(=O)c1ccccc1NCC(=O)O)c1ccccc1]

51. The bifunctional compound according to claim 50, wherein S_{L} is a substituent derived from the following compound:

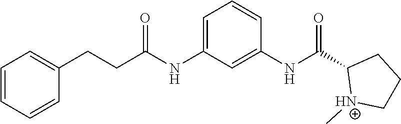 [SMILES: C[NH+]1CCC[C@H]1C(=O)Nc1cccc(NC(=O)CCc2ccccc2)c1]

52. The bifunctional compound according to claim 50, wherein S_{L} is a substituent derived from the following compound:

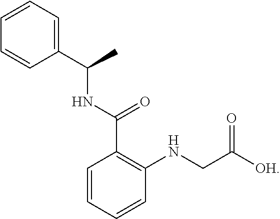 [SMILES: C[C@@H](NC(=O)c1ccccc1NCC(=O)O)c1ccccc1]

53. The bifunctional compound according to claim 1, wherein S_{L} is a substituent derived from a compound of Formula (VIII) or Formula (IX): 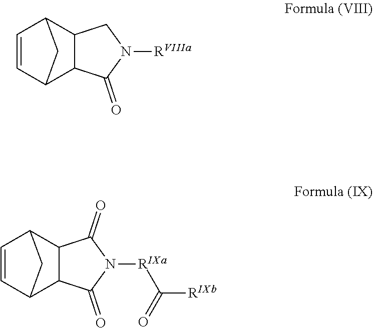
wherein,
R^{VIIIa} is a carboxylic acid or ester thereof and where any of the carbons of the norbornene ring can be substituted with a C₁₋₆ alkyl group or the carboxylic acid can be condensed as a C₁₋₅ ester,
wherein R^{IXa} is an optionally substituted bivalent C₁-C₅ alkyl, wherein one or more methylene groups is optionally replace by —CO(NH)—,
R^{IXb} is a group selected from the group consisting of hydroxyl, alkoxy, amino or aminoalkyl.

54. The bifunctional compound according to claim 53, wherein the compound of Formula (VIII) or (IX) is selected from
2-methyl-3,5-dioxo-4-azatricyclo[5.2.1.0 (2,6)]dec-8-en-4-yl) acetic acid, methyl 2-(1,3-dioxo-1,3,3a,4,7,7a-hexahydro-2H-4,7-methanoisoindol-2-yl) propanoate,
2-(1,3-dioxo-1,3,3a,4,7,7a-hexahydro-2H-4,7-methanoisoindol-2-yl)-4-(methylthio)butanoic acid, and
2-(3,5-dioxo-4-azatricyclo[5.2.1.0 (2,6)]dec-8-en-4-yl)-4-methylpentanoic acid.

55. The bifunctional compound according to claim 1, wherein S_{L} is a substituent derived from a compound of Formula (X): 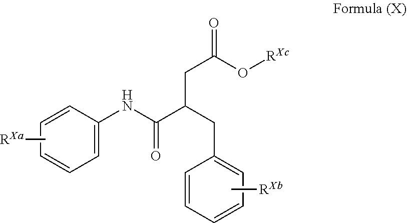
wherein
R^{Xa} and R^{Xb} are independently C₁₋₅ alkyl, acyl, amino, sulfono, chloro, bromo, iodo, or flouro; and
R^{Xc} is C₁₋₅ alkyl or the acid is replaced with a tetrazol.

56. The bifunctional compound according to claim 55, wherein the compound of Formula (X) is 4-[(3,4-dichlorophenyl)amino]-3-(3-methylbenzyl)-4-oxobutanoic acid or 3-benzyl-4-[(3-chloro-2-methylphenyl)amino]-4-oxobutanoic acid.

57. The bifunctional compound according to claim 1, wherein S_{L} is a substituent derived from Formula (XI): 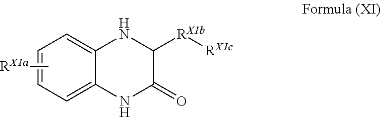
wherein
R^{XIa} is C₁₋₅ alkyl, acyl, amino, sulfono, chloro, bromo, iodo, or fluoro;
R^{XIb} is a bond or a C₁-C₃ alkyl optionally substituted with a C₁-C₅ alkyl and
R^{XIc} is a carboxylic acid, a C₁-C₅ ester of a carboxylic acid or a tetrazole.

58. The bifunctional compound according to claim 56, wherein the compound of Formula (XI) is 3-oxo-1,2,3,4-tetrahydro-2-quinoxalinyl) acetic acid.

59. The bifunctional compound according to claim 1, wherein S_{L} is a substituent derived from Formula (A): 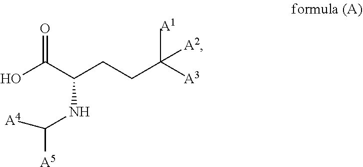
wherein
A¹, A² and A³ are each independently selected from the group consisting of halogen, H, C₁-C₄ alkyl, C₁-C₄ haloalkyl, C₂-C₅ alkenyl and C₂-C₅ haloalkenyl;
A⁴ is selected from the group consisting of H, C₁-C₃ alkyl, C₁-C₈ haloalkyl, C₃-C₈ aryl, C₃-C₈ aryl with one or more halogen substituents, C₃-C₈ heteroaryl and C₃-C₈ heteroaryl with one or more halogen substituents;
A⁵ is selected from the group consisting of C₃-C₂₀ aryl, C₃-C₂₀ heteroaryl and 3- to 12-membered-heterocyclic ring; wherein the aryl, heteroaryl or heterocyclic ring is optionally substituted with one or more substituents independently selected from halogen, —OH, cyano, carbonyl, C₁-C₄ alkyl, C₁-C₄ hydroxyalkyl, C₁-C₄ haloalkyl, acetyl, C₁-C₄ alkoxy, C₁-C₄ haloalkoxy, C₃-C₈ aryl and C₃-C₈ heteroaryl; or
A⁴ and A⁵ are taken together to form a 6- to 20 membered heterocyclic ring, wherein wherein the heterocyclic ring is monocyclic, bicyclic or tricyclic and is optionally substituted with one or more substituents independently selected from halo, —OH, cyano, carbonyl, C₁-C₄ alkyl, C₁-C₄ haloalkyl, acetyl, C₁-C₄ haloalkoxy, C₁-C₄ haloalkoxy.

60. The bifunctional compound according to claim 1 or 59, wherein S_{L} is according to any one of formulas A-I to A-III: 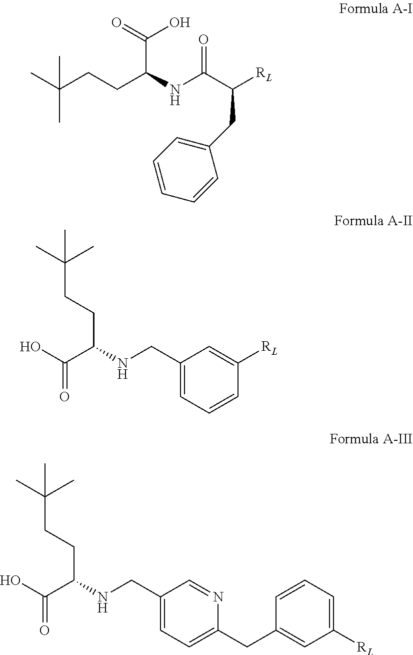
or a derivative thereof, wherein R^{L} denotes the attachment to L_{I}.

61. The bifunctional compound according to claim 1, wherein S_{L} comprises a peptide.

62. The bifunctional compound according to claim 61, wherein S_{L} comprises a peptide with a length of no more than 50 amino acid residues, such as no more than 45, such as no more than 40, such as no more than 35, such as no more than 32, such as no more than 30, such as no more than 28, such as no more than 26, such as no more than 24, such as no more than 22, such as no more than 20, such as no more than 19, such as no more than 18, such as no more than 17, such as no more than 16, such as no more than 15, such as no more than 14, such as no more than 13, such as no more than 12, such as no more than 11, such as no more than 10, such as no more than 9, such as no more than 8, such as no more than 7, such as no more than 6, such as no more than 5, such as no more than 4 amino acid residues.

63. The bifunctional compound according to any one of claim 61 or 62, wherein S_{L} comprises a peptide that comprises at least 4 amino acid residues, such as at least 5, such as at least 6, such as at least 7, such as at least 8, such as at least 9, such as at least 10, such as at least 12, such as at least 14, such as at least 16, such as at least 18, such as at least 20, such as at least 22, such as at least 24, such as at least 26, such as at least 28 amino acid residues.

64. The bifunctional compound according to any one of claims 61 to 63, wherein S_{L} comprises or consist of a peptide that comprises between 4 and 32 amino acids.

65. The bifunctional compound according to any one of claims 61 to 63, wherein S_{L} comprises a peptide that comprises 4 or 5 amino acids.

66. The bifunctional compound according to claim 1 or any one of claims 61 to 65, wherein S_{L} comprises a peptide having a length of 4 to 50 amino acids comprising a fragment of an amino acid sequence of a protein selected from the group consisting of:
a. progranulin (SEQ ID NO.: 3),
b. neurotensin (SEQ ID NO.: 4),
c. brain derived neurotrophic factor (BDNF) (SEQ ID NO.: 177)
d. apolipoprotein B (ApoB) (SEQ ID NO.: 176)
e. nerve growth factor (NGF) (SEQ ID NO.: 178)
or a variant of said fragment, having at least 60% sequence identity with the corresponding original protein fragment of any one of a) through e), such as at least 80%, such as at least 90%, such as at least 95% sequence identity.

67. The bifunctional compound according to claim 1 or any one of claims 61 to 66, wherein S_{L} comprises or consists of a peptide fragment of an amino acid sequence of progranulin (SEQ ID NO.: 3), or a variant of said fragment having at least 60% sequence identity with the corresponding original fragment of SEQ ID NO.: 3, such as at least 80% sequence identity, such as at least 90%, such as at least 95%, such as at least 99% sequence identity.

68. The bifunctional compound according to claim 1 or any one of claims 61 to 66, wherein S_{L} comprises or consists of a peptide fragment of an amino acid sequence of progranulin (SEQ ID NO.: 3), or a variant of said fragment having up to 5 amino acids substitutions compared to the corresponding original fragment of SEQ ID NO.: 3, such up to 4 amino acid substitutions, such as up to 3 amino acid substitutions, such as up to 2 amino acids substitutions, such as up to 1 amino acid substitution.

69. The bifunctional compound according to claim 1 or any one of claims 61 to 66, wherein S_{L} comprises or consists of a peptide fragment of an amino acid sequence of neurotensin (SEQ ID NO.: 4), or a variant of said fragment having at least 60% sequence identity with the corresponding original fragment of SEQ ID NO.: 4, such as at least 80% sequence identity, such as at least 90%, such as at least 95%, such as at least 99% sequence identity.

70. The bifunctional compound according to claim 1 or any one of claims 61 to 66, wherein S_{L} comprises or consists of a peptide fragment of an amino acid sequence of neurotensin (SEQ ID NO.: 4), or a variant of said fragment having up to 5 amino acids substitutions compared to the corresponding original fragment of SEQ ID NO.: 4, such up to 4 amino acid substitutions, such as up to 3 amino acid substitutions, such as up to 2 amino acids substitutions, such as up to 1 amino acid substitution.

71. The bifunctional compound according to claim 1 or any one of claims 61 to 66, wherein S_{L} comprises or consists of a sequence selected from the group consisting of:
```
  (SEQ ID NO.: 44)
H-PYMKLAPGELTIIL-OH,
  (SEQ ID NO.: 45)
H-NEKLSQLQTYMI-OH,
  (SEQ ID NO.: 46)
H-KDADLYTSRVMLSSQVP-OH,
  (SEQ ID NO.: 49)
H-ITVDPRLFKKRRLRSPRVLFSTQPPR-OH,
  (SEQ ID NO.: 50)
H-WSGPIGVSWGLRAAAAGGAFP-OH,
  (SEQ ID NO.: 51)
H-WSGPIGVSWGLRAAAAGGAFPRGGRWRR-OH,
  (SEQ ID N.: 52)
H-GVSWGLR-OH
  (SEQ ID NO.: 179)
H-WSGPIGVSWGLRAAAAGFQLL-OH.
```

72. The bifunctional compound according to claim 1, wherein S_{L} comprises a peptide comprising the following sequence:
(SEQ ID NO.: 16)
X₅-X₁X₂X₃X₄
wherein,
X₅ is an optional amino acid residue or peptide comprising 2 to 30 amino acid residues or a bond,
X₁ is R, P, F, Y, L, K, G or H;
X₂ is Q, Y, L, E or G;
X₃ is Y, F, L, I, Q, E or N;
X₄ is M, K, L or a conservative substitution of L; and
L_{I} is conjugated to the N-terminus.

73. The bifunctional compound according to claim 72, wherein X₅ is a peptide comprising at least 2 amino acid residues, such as at least 3, such as at least 4, such as at least 5, such as at least 6, such as at least 7, such as at least 8, such as at least 9, such as at least 10, such as at least 11, such as at least 12, such as at least 13, such as at least 14, such as at least 15, such as at least 20, such as at least 25 amino acid residues, such as at least 28 amino acid residues, such as at least 30 amino acid residues.

74. The bifunctional compound according to claim 72, wherein X₅ is a peptide comprising no more than 32 amino acid residues, such as no more than 29, such as no more than 28, such as no more than 27, such as no more than 26, such as no more than 25, such as no more than 24, such as no more than 23, such as no more than 22, such as no more than 21, such as no more than 20, such as no more than 15, such as no more than 10, such as no more than 5 amino acid residues.

75. The bifunctional compound according to any one of claims 72 to 74, wherein X₅ is a fragment of an amino acid sequence of a protein selected from the group consisting of:
a. progranulin (SEQ ID NO.: 3),
b. neurotensin (SEQ ID NO.: 4),
c. brain derived neurotrophic factor (BDNF)
d. apolipoprotein B (ApoB)
e. nerve growth factor (NGF)
or a variant of said fragment, having at least 60% sequence identity with the corresponding original protein fragment of any one of a) through e), such as at least 80%, such as at least 90%, such as at least 95% sequence identity.

76. The bifunctional compound according to any one of claims 72 to 75, wherein X₅ is comprises or consists of a peptide fragment of an amino acid sequence of progranulin (SEQ ID NO.: 3), or a variant of said fragment having at least 60% sequence identity with the corresponding original fragment of SEQ ID NO.: 3, such as at least 80% sequence identity, such as at least 90%, such as at least 95%, such as at least 99% sequence identity.

77. The bifunctional compound according to any one of claims 72 to 75, wherein X₅ comprises or consists of a peptide fragment of an amino acid sequence of progranulin (SEQ ID NO.: 3), or a variant of said fragment having up to 5 amino acids substitutions compared to the corresponding original fragment of SEQ ID NO.: 3, such up to 4 amino acid substitutions, such as up to 3 amino acid substitutions, such as up to 2 amino acids substitution, such as up to 1 amino acid substitution.

78. The bifunctional compound according to any one of claims 72 to 75, wherein X₅ is comprises or consists of a peptide fragment of an amino acid sequence of neurotensin (SEQ ID NO.: 3), or a variant of said fragment having at least 60% sequence identity with the corresponding original fragment of SEQ ID NO.: 3, such as at least 80% sequence identity, such as at least 90%, such as at least 95%, such as at least 99% sequence identity.

79. The bifunctional compound according to any one of claims 72 to 75, wherein X₅ comprises or consists of a peptide fragment of an amino acid sequence of neurotensin (SEQ ID NO.: 4), or a variant of said fragment having up to 5 amino acids substitutions compared to the corresponding original fragment of SEQ ID NO.: 4, such up to 4 amino acid substitutions, such as up to 3 amino acid substitutions, such as up to 2 amino acids substitution, such as up to 1 amino acid substitution.

80. The bifunctional compound according any one of claims 72 to 75, wherein X₅ is selected from the group consisting of:
a bond
an amino acid residue selected from L, T or Y;
```
  (SEQ ID NO.: 17)
REAPRWDAPLRDPAL;
  (SEQ ID NO.: 18)
REALRWDAPLRDPAP;
  (SEQ ID NO.: 19)
PYILKRQLYENKPRR;
  (SEQ ID NO.: 20)
LYENKPR;
and
  (SEQ ID NO.: 21)
APLRDPAP
  (SEQ ID NO.: 180)
WSGPIGVSWGLRAAAAG
  (SEQ ID NO.: 181)
CREAPRWDAPLRDPAL
```
or a variant thereof having 1 to 5 amino acids substitutions.

81. The bifunctional compound according any one of claims 72 to 80, wherein X₅ is a bond.

82. The bifunctional compound according any one of claims 72 to 80, wherein X₅ is
(SEQ ID NO.: 21)
APLRDPAP

83. The bifunctional compound according any one of claims 72 to 80, wherein X₅ is
(SEQ ID NO.: 17)
REAPRWDAPLRDPAL.

84. The bifunctional compound according any one of claims 72 to 80, wherein X₅ is
(SEQ ID NO.: 18)
REALRWDAPLRDPAP.

85. The bifunctional compound according to any one of claim 72 or 84, wherein
X₁ is selected from Y, F, R, P, L or H,
X₂ is selected from Q, Y or L,
X₃ is selected from Y, F, L, I or Q, and
X₄ is L.

86. The bifunctional compound according to any one of claim 72 or 85, wherein
X₁ is selected from Y, F, R or P,
X₂ is selected from Q or Y,
X₃ is selected from L, Y, F or I, and
X₄ is L.

87. The bifunctional compound according to any one of claims 72 to 86, wherein X₁ is selected from Y, F or R,
X₂ is selected from Q or Y,
X₃ is L, and
X₄ is L.

88. The bifunctional compound according to any one of claims 72 to 87, wherein X₁ is F or R.

89. The bifunctional compound according to any one of claims 72 to 88, wherein X₂ is Q.

90. The bifunctional compound according to any one of claims 72 to 89, wherein X₃ is Q.

91. The bifunctional compound according to any one of claims 72 to 90, wherein X₄ is L.

92. The bifunctional compound according to any one of claims 72 to 91, wherein X₅ is a bond.

93. The bifunctional compound according to any one of claims 72 to 92, wherein
X₁ is selected from F or R,
X₂ is Q,
X₃ is L,
X₄ is L.

94. The bifunctional compound according to any one of claims 72 to 92, wherein the peptide according to SEQ ID NO.: 16 is located at the C-terminus of S_{L}.

95. The bifunctional compound according to any one of claims 1 or 72 to 94, wherein S_{L} comprises or consists of a peptide selected from the group consisting of:
```
  (SEQ ID NO.: 22)
RQLL-OH,
  (SEQ ID NO.: 23)
FQLL-OH,
  (SEQ ID NO.: 24)
KQLL-OH,
  (SEQ ID NO.: 25)
PQLL-OH,
  (SEQ ID NO.: 26)
PYIL-OH,
  (SEQ ID NO.: 27)
RYLL-OH,
  (SEQ ID NO.: 28)
FYLL-OH,
  (SEQ ID NO.: 29)
YQLL-OH,
  (SEQ ID NO.: 30)
LLQL-OH,
  (SEQ ID NO.: 31)
FYIL-OH,
  (SEQ ID NO.: 32)
RYIL-OH,
  (SEQ ID NO.: 33)
PYLL-OH,
  (SEQ ID NO.: 34)
PYYL-OH,
  (SEQ ID NO.: 35)
PYFL-OH,
  (SEQ ID NO.: 36)
RYYL-OH,
  (SEQ ID NO.: 37)
RYFL-OH,
  (SEQ ID NO.: 38)
RQFL-OH,
  (SEQ ID NO.: 39)
RQYL-OH,
  (SEQ ID NO.: 40)
LQLL-OH,
  (SEQ ID NO.: 41)
HQLL-OH,
  (SEQ ID NO.: 42)
IQLL-OH,
  (SEQ ID NO.: 43)
FYYL-OH,
  (SEQ ID NO.: 44)
PYMKLAPGELTIIL-OH,
  (SEQ ID NO.: 45)
NEKLSQLQTYMI-OH,
  (SEQ ID NO.: 46)
KDADLYTSRVMLSSQVP-OH,
  (SEQ ID NO.: 49)
ITVDPRLFKKRRLRSPRVLFSTQPPR-OH,
  (SEQ ID NO.: 50)
WSGPIGVSWGLRAAAAGGAFP-OH,
  (SEQ ID NO.: 51)
WSGPIGVSWGLRAAAAGGAFPRGGRWRR-OH,
  (SEQ ID NO.: 52)
GVSWGLR-OH,
  (SEQ ID NO.: 53)
REAPRWDAPLRDPALROLL-OH,
  (SEQ ID NO.: 54)
REALRWDAPLRDPAPRQLL-OH,
  (SEQ ID NO.: 55)
PYILKRQLYENKPRRPYIL-OH,
  (SEQ ID NO.: 56)
LYENKPRRPYIL-OH,
  (SEQ ID NO.: 57)
APLRDPAPRQLL-OH,
  (SEQ ID NO.: 58)
REAPRWDAPLRDPALFQLL-OH,
  (SEQ ID NO.: 59)
REAPRWDAPLRDPALRYLL-OH,
  (SEQ ID NO.: 60)
REAPRWDAPLRDPALRQYL-OH,
  (SEQ ID NO.: 61)
REAPRWDAPLRDPALRYYL-OH,
  (SEQ ID NO.: 62)
TGGFM-OH,
and
  (SEQ ID NO.: 63)
YGGFL-OH,
  (SEQ ID NO.: 182)
WSGPIGVSWGLRAAAAGFQLL-OH,
  (SEQ ID NO.: 183)
WSGPIGVSWGLRAAAAGRQLL-OH,
  (SEQ ID NO.: 184)
CREAPRWDAPLRDPALRQLL-OH,
  (SEQ ID NO.: 186)
CREAPRWDAPLRDPALFQLL-OH
```
and
wherein L_{I} is connected at the N-terminus.

96. The bifunctional compound according to any one of claims 1 or 72 to 94, wherein S_{L} comprises or consists of a peptide according to any one of sequences:
```
  (SEQ ID NO.: 22)
RQLL-OH,
  (SEQ ID NO.: 23)
FQLL-OH,
  (SEQ ID NO.: 27)
RYLL-OH,
  (SEQ ID NO.: 28)
FYYL-OH,
  (SEQ ID NO.: 29)
YQLL-OH,
  (SEQ ID NO.: 53)
REAPRWDAPLRDPALRQLL-OH,
  (SEQ ID NO.: 54)
REALRWDAPLRDPAPRQLL-OH,
  (SEQ ID NO.: 58)
REAPRWDAPLRDPALFQLL-OH,
  (SEQ ID NO.: 59)
REAPRWDAPLRDPALRYLL-OH,
  (SEQ ID NO.: 60)
REAPRWDAPLRDPALRQYL-OH,
  (SEQ ID NO.: 61)
REAPRWDAPLRDPALRYYL-OH,
  (SEQ ID NO.: 57)
APLRDPAPRQLL-OH,
  (SEQ ID NO.: 55)
PYILKRQLYENKPRRPYIL-OH,
  (SEQ ID NO.: 56)
LYENKPRRPYIL-OH,
```
wherein L_{I} is connected at the N-terminus.

97. The bifunctional compound according to claim 1, wherein S_{L} comprises an amino acid sequence selected from the group consisting of:
```
  (SEQ ID NO.: 7)
IKLSGGVQAKAGVINMDKSESM
  (SEQ ID NO.: 8)
IKLSGGVQAKAGVINMFKSESY
  (SEQ ID NO.: 9)
IKLSGGVQAKAGVINMFKSESYK
  (SEQ ID NO.: 10)
GVQAKAGVINMFKSESY
  (SEQ ID NO.: 11)
GVRAKAGVRNMFKSESY
  (SEQ ID NO.: 12)
GVRAKAGVRN(Nle)FKSESY
  (SEQ ID NO.: 13)
YKSLRRKAPRWDAPLRDPALRQLL
  (SEQ ID NO.: 14)
YKSLRRKAPRWDAYLRDPALRQLL
  (SEQ ID NO.: 15)
YKSLRRKAPRWDAYLRDPALRPLL
```

98. The bifunctional compound according to any one of claims 61 to 97, wherein one to four amino acid residues of S_{L} are conservatively substituted.

99. The bifunctional compound according to any one of claims 61 to 98, wherein one to four amino acid residues of S_{L} are substituted with a non-naturally occurring amino acid.

100. The bifunctional compound according to any one of claims 61 to 97, wherein one to four of the amino acid residues of S_{L} are modified amino acid residues.

101. The bifunctional compound according to any one of claims 61 to 100, wherein the peptide is a modified peptide such as by acylation, amidation, acetylation, esterification and/or alkylation.

102. The bifunctional compound according to any one of the preceding claims, wherein S_{L} is a peptide able to bind sortilin with a dissociation constant to sortilin of less than 50 μM, such as less than 2 μM, such as less than 0.5 μM, preferably less than 0.1 μM.

103. A peptide comprising or consisting of a sequence selected from the group consisting of:
(SEQ ID NO.: 22)
RQLL-OH,
(SEQ ID NO.: 23)
FQLL-OH,
(SEQ ID NO.: 24)
KQLL-OH,
(SEQ ID NO.: 25)
PQLL-OH,
(SEQ ID NO.: 26)
PYIL-OH,
(SEQ ID NO.: 27)
RYLL-OH,
(SEQ ID NO.: 28)
FYLL-OH,
(SEQ ID NO.: 29)
YQLL-OH,
(SEQ ID NO.: 30)
LLQL-OH,
(SEQ ID NO.: 31)
FYIL-OH,
(SEQ ID NO.: 32)
RYIL-OH,
(SEQ ID NO.: 33)
PYLL-OH,
(SEQ ID NO.: 34)
PYYL-OH,
(SEQ ID NO.: 35)
PYFL-OH,
(SEQ ID NO.: 36)
RYYL-OH,
(SEQ ID NO.: 37)
RYFL-OH,
(SEQ ID NO.: 38)
RQFL-OH,
(SEQ ID NO.: 39)
RQYL-OH,
(SEQ ID NO.: 40)
LQLL-OH,
(SEQ ID NO.: 41)
HQLL-OH,
(SEQ ID NO.: 42)
IQLL-OH,
(SEQ ID NO.: 43)
FYYL-OH,
(SEQ ID NO.: 44)
PYMKLAPGELTIIL-OH,
(SEQ ID NO.: 45)
NEKLSQLQTYMI-OH,
(SEQ ID NO.: 46)
KDADLYTSRVMLSSQVP-OH,
(SEQ ID NO.: 47)
RLFKKRRLRSPRVLF-NH2,
(SEQ ID NO.: 48)
ITVDPRLFKKRRLRSPRVLF-NH2,
(SEQ ID NO.: 49)
ITVDPRLFKKRRLRSPRVLFSTQPPR-OH,
(SEQ ID NO.: 50)
WSGPIGVSWGLRAAAAGGAFP-OH,
(SEQ ID NO.: 51)
WSGPIGVSWGLRAAAAGGAFPRGGRWRR-OH,
(SEQ ID NO.: 52)
GVSWGLR-OH,
(SEQ ID NO.: 53)
REAPRWDAPLRDPALRQLL-OH,
(SEQ ID NO.: 54)
REALRWDAPLRDPAPRQLL-OH,
(SEQ ID NO.: 55),
PYILKRQLYENKPRRPYIL-OH,
(SEQ ID NO.: 56)
LYENKPRRPYIL-OH,
(SEQ ID NO.: 57)
APLRDPAPRQLL-OH,
(SEQ ID NO.: 58)
REAPRWDAPLRDPALFQLL-OH,
(SEQ ID NO.: 59)
REAPRWDAPLRDPALRYLL-OH,
(SEQ ID NO.: 60)
REAPRWDAPLRDPALRQYL-OH,
(SEQ ID NO.: 61)
REAPRWDAPLRDPALRYYL-OH,
(SEQ ID NO.: 62)
TGGFM-OH,
and
(SEQ ID NO.: 63)
YGGFL-OH,
(SEQ ID NO.: 64)
LYEN-NH3,
and
(SEQ ID NO.: 65)
LYENK-NH3
(SEQ ID NO.: 182)
WSGPIGVSWGLRAAAAGFQLL-OH;
(SEQ ID NO.: 183)
WSGPIGVSWGLRAAAAGRQLL-OH;
(SEQ ID NO.: 184)
CREAPRWDAPLRDPALRQLL-OH;
and
(SEQ ID NO.: 186)
CREAPRWDAPLRDPALFQLL-OH.

104. The peptide according to claim 103 wherein the peptide comprises or consists of the sequence RQLL-OH (SEQ ID NO.: 22).

105. The peptide according to claim 103 wherein the peptide comprises or consists of the sequence FQLL-OH (SEQ ID NO.: 23).

106. The peptide according to claim 103 wherein the peptide comprises or consists of the sequence RYLL-OH (SEQ ID NO.: 27).

107. The peptide according to claim 103 wherein the peptide comprises or consists of the sequence FYLL-OH (SEQ ID NO.: 28).

108. The peptide according to claim 103 wherein the peptide comprises or consists of the sequence YQLL-OH (SEQ ID NO.: 29).

109. The peptide according to claim 103 wherein the peptide comprises or consists of the sequence REAPRWDAPLRDPALROLL-OH (SEQ ID NO.: 53).

110. The peptide according to claim 103 wherein the peptide comprises or consists of the sequence REALRWDAPLRDPAPRQLL-OH (SEQ ID NO.: 54).

111. The peptide according to claim 103 wherein the peptide comprises or consists of the sequence REAPRWDAPLRDPALFQLL-OH (SEQ ID NO.: 58).

112. The peptide according to claim 103 wherein the peptide comprises or consists of the sequence REAPRWDAPLRDPALRYLL-OH (SEQ ID NO.: 59).

113. The peptide according to claim 103 wherein the peptide comprises or consists of the sequence REAPRWDAPLRDPALRQYL-OH (SEQ ID NO.: 60).

114. The peptide according to claim 103 wherein the peptide comprises or consists of the sequence REAPRWDAPLRDPALRYYL (SEQ ID NO.: 61).

115. The peptide according to claim 103 wherein the peptide comprises or consists of the sequence APLRDPAPRQLL-OH (SEQ ID NO.: 57).

116. The peptide according to claim 103 wherein the peptide comprises or consists of the sequence PYILKRQLYENKPRRPYIL-OH (SEQ ID NO.: 55).

117. The peptide according to claim 103 wherein the peptide comprises or consists of the sequence LYENKPRRPYIL-OH (SEQ ID NO.: 56).

118. The peptide according to any one of claims 103 to 104, wherein the peptide is able to bind sortilin with a dissociation constant to of less than 50 μM, such as less than 2 μM, such as less than 0.5 μM, preferably less than 0.1 μM.

119. The bifunctional compound according to claim 1, wherein S_{L} is a protein.

120. The bifunctional compound according to claim 119, wherein S_{L} is an antibody, an antibody fragment or a nanobody.

121. The bifunctional compound according to any one of claims 119 to 120, wherein the bifunctional compound is able to bind sortilin.

122. The bifunctional compound according to any one of the preceding claims wherein S_{L} is derived from a protein, peptide or a compound able to bind sortilin with a dissociation constant of less than of less than 50 μM, such as less than 40 μM, such as less than 30 μM, such as less than 20 μM, such as less than 10 μM, such as less than 5 μM, such as less than 4 μM, such as less than 3 μM, such as less than 2 μM, such as less than 1 μM, such as less than 0.8 μM, such as less than 0.6 μM, such as less than 0.5 μM, such as less than 0.4 μM, such as less than 0.3 μM, such as less than 0.2 μM, such as less than 0.1 μM.

123. The bifunctional compound according to any one of the preceding claims wherein the bifunctional compound is able to bind sortilin with a dissociation constant of less than 50 μM, such as less than 40 μM, such as less than 30 μM, such as less than 20 μM, such as less than 10 μM, such as less than 5 μM, such as less than 4 μM, such as less than 3 μM, such as less than 2 μM, such as less than 1 μM, such as less than 0.8 μM, such as less than 0.6 μM, such as less than 0.5 μM, such as less than 0.4 μM, such as less than 0.3 μM, such as less than 0.2 μM, such as less than 0.1 μM, such as less than 0.05 μM, such as less than 0.04 μM, such as less than 0.03 μM, such as less than 0.02 μM such as less than 0.01 μM.

124. The bifunctional compound according to any one of the preceding claims wherein the bifunctional compound is able to bind sortilin with a dissociation constant between 50 μM and 0.001 μM, such as between 50 μM and 0.001 μM, such as between 50 μM and 40 μM, such as between 40 μM and 30 μM, such as between 30 μM and 20 μM, such as between 20 μM and 10 μM, such as between 10 μM and 5 μM, such as between 5 μM and 4 μM, such as between 4 μM and 3 μM, such as between 3 μM and 2 μM, such as between 2 μM and 1 μM, such as between 1 μM and 0.9 μM, such as between 0.9 μM and 0.8 μM, such as between 0.8 μM and 0.7 μM, such as between 0.7 μM and 0.6 μM, such as between 0.5 μM and 0.4 μM, such as between 0.4 μM and 0.3 μM, such as between 0.3 μM and 0.2 μM, such as between 0.2 μM and 0.1 μM, such as between 0.1 μM and 0.05 μM, such as between 0.05 μM and 0.01 μM, such as between 0.01 μM and 0.001 μM.

125. An isolated polynucleotide encoding for the peptide according to any one of claims 61 to 118 or the protein according to any one of claims 119 to 120.

126. A vector comprising the polynucleotide according to claim 125.

127. The vector according to claim 126, wherein the vector is an expression vector, such as a bacterial vector or a viral vector.

128. A host cell comprising the polynucleotide according to claim 127 and/or the vector according to any one of claims 128 to 129.

129. The bifunctional compound according to any one of the claims 1 to 124 wherein the extracellular target molecule is a target protein.

130. The bifunctional compound according to any one of the claims 1 to 124, wherein the target molecule is TNFα.

131. The bifunctional compound according to claim 129, wherein the target protein is selected from the group consisting of: TNF-α, ANGPTL-3, an antibody light chain, IgG, IgE, IgA IL-1, IL-2, IL-6, IFN-γ, VEGF, TFG-β1, IL-21, IL-22, IL-5, IL-10, IL-8, cholinestearase, human CCL2, carboxypeptidase B-2, neutrophil elastase, Factor Xa, Factor XI, Factor XIa, Factor XII, Factor XIII, prothrombin, coagulation factor VII, coagulation factor IX, fibroblast growth factor 1, FGF-2, fibronectin 1, kallikrein-1, lipoprotein lipase, human matrix metallopeptidase 1, macrophage migration inhibitory factor, transformin growth factor-p (TGF-p), thrombospondin-1 (TSP-T), CD40 ligand, urokinase-type plasminogen activator, plasminogen activator tissue type (TPA), Plasminogen (PLG), Plasminogen Activator Inhibitor-1, Placenta Growth Factor, Phospholipase A2 Group IB, Phospholipase A2 Group IIA, Complement factor B, Complement factor D, complement factor H, Complement Component 5 and complement C1s.

132. The bifunctional compound according to claim any one of claims 129 to 131, wherein the bifunctional compound binds the target molecule or protein through T_{L}.

133. The bifunctional compound according to claim 129, wherein T_{L} is derived from a compound, peptide or protein able to bind the target protein with a dissociation constant of less than 100 μM, such as less than 2 μM, such as less than 0.5 μM, preferably less than 0.1 μM.

134. The bifunctional compound according to any one of claims 1 to 124, wherein T_{L} is a substituent derived from biotin.

135. The bifunctional compound according to any one of claims 1 to 124, wherein T_{L} is according to formula (XII): 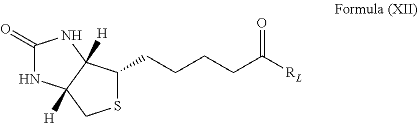
wherein R^{L} denotes the attachment with L_{I}.

136. The bifunctional compound according to any one of claims 1 to 124, wherein T_{L} is a substituent derived from dinitrophenyl.

137. The bifunctional compound according to any one of claims 1 to 124, wherein T_{L} is according to formula (XII): 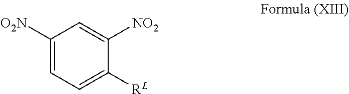
wherein R^{L} denotes the attachment with L_{I}.

138. The bifunctional compound according to any one of claims 1 to 124, wherein T_{L} is according to formula XVIIa, 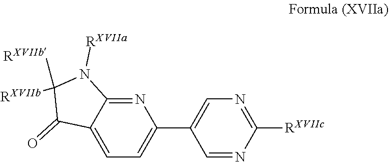
wherein, R^{XVIIa} has the formula 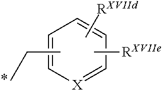
wherein R^{XVIId} and R^{XVIIe} are independently selected from the group consisting of C₁-C₅ alkyl, C₁-C₅ alkoxy, cyano, halogen and C₁ haloalkyl, each of which is optionally independently substituted; X is an atom selected from N or CH and * denotes the attachment with formula (XVIIa);
R^{XVIIb} and R^{XVIIb′} are each independently selected from H and a C₁-C₃ alkyl;
R^{XVIIc} is R_{L} or is selected from the group consisting of formula XVIIa-1 and formula XVIIa-2: 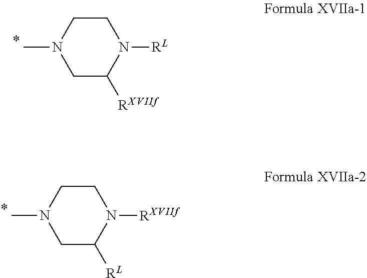
wherein R^{XVIIf} is selected from the group consisting of an optionally substituted C₁-C₅ alkyl wherein one or more of the methylene groups is replaced by one selected from the group consisting of carbonyl, ester, amide, —NH— or —O—, and * denotes the attachment with formula XVIIa; and
R_{L} denotes the attachment with L_{I}.

139. The bifunctional compound according to claim 138, wherein R^{XVIIb} or R^{XVIIb′} are —CH₃.

140. The bifunctional compound according to claim 138, wherein R^{XVIIb} and R^{XVIIb′} are —CH₃.

141. The bifunctional compound according to any one of claims 137 to 140, wherein R^{XVIIa} is selected from the group consisting of: 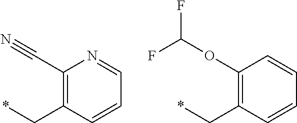
wherein * denotes the attachment with formula XVIIa.

142. The bifunctional compound according to any one of claims 1 to 124 or 137 to 141, wherein T_{L} is according to formula XVIIa-3: 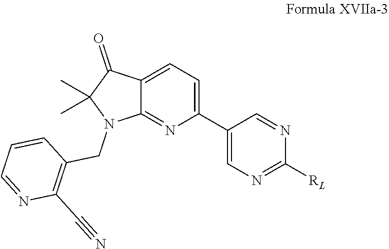
wherein R_{L} denotes attachment with L_{I}.

143. The bifunctional compound according to any one of claims 1 to 124 or 137 to 141, wherein T_{L} is according to formula XVIIa-4: 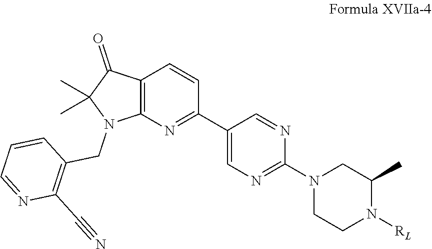
wherein R_{L} denotes attachment with L_{I}.

144. The bifunctional compound according to any one of claims 1 to 124 or 137 to 141, wherein T_{L} is according to formula XVIIa-5: 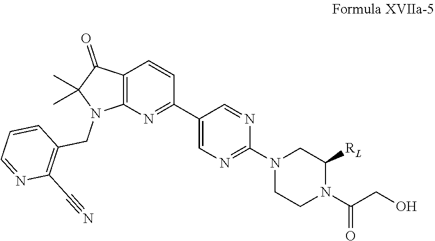
wherein R_{L} denotes attachment with L_{I}.

145. The bifunctional compound according to any one of claims 1 to 124, wherein T_{L} is according to formula XVIIb 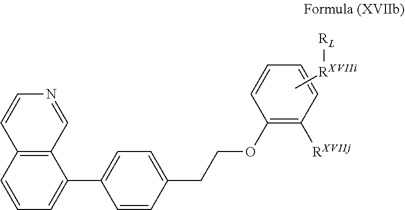
wherein R^{XVIIi} is selected from a bond and an optionally substituted piperazine group;
R^{XVIIj} is selected from the group consisting of 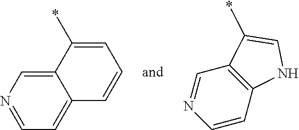 [SMILES: *c1cccc2ccncc12 ; *c1c[nH]c2ccncc12]
wherein * denotes attachment with formula XVIIb; and
wherein R_{L} denotes attachment with L_{I}.

146. The bifunctional compound according to claim 145, wherein T_{L} is according to formula XVIIb-1: 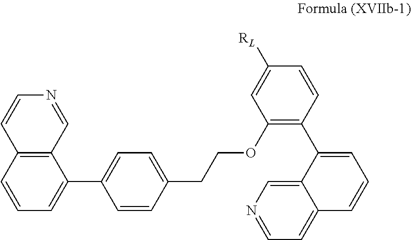
wherein R_{L} denotes attachment with L_{I}.

147. The bifunctional compound according to claim 145, wherein T_{L} is according to formula XVIIb-2: 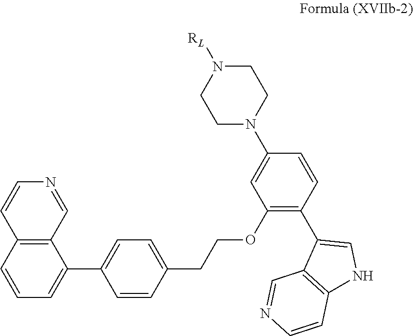
wherein R_{L} denotes attachment with L_{I}.

148. The bifunctional compound according to any one of claims 1 to 124, wherein T_{L} is according to formula XVIIc 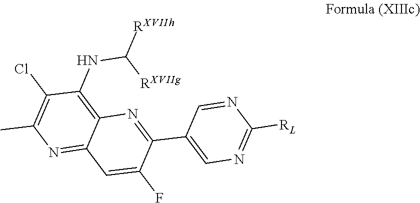
wherein R^{XVIIg} is a C₁-C₄ alkyl and R^{XVIIh} is an aromatic or heteroaromatic ring optionally substituted with one or more of the group selected from halogen, haloalkyl, cyano, hydroxyl, amino, hydroxyl, alkoxy, C₃-C₆ cycloalkyl and C₃-C₆ heterocycloalkyl; and
wherein R_{L} denotes attachment with L_{I}.

149. The bifunctional compound according to claim 148, wherein R^{XVIIg} is —CH₃.

150. The bifunctional compound according to any one of claims 148 to 149, wherein T_{L} is according to formula XVIIc-1 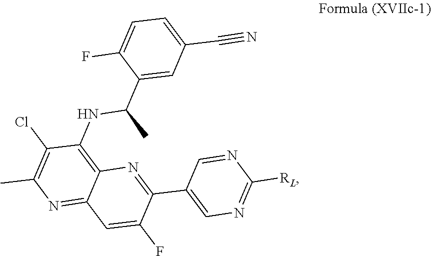
wherein R_{L} denotes attachment with L_{I}.

151. The bifunctional compound according to any one of claims 1 to 124, wherein T_{L} is according to formula XVIIc-2 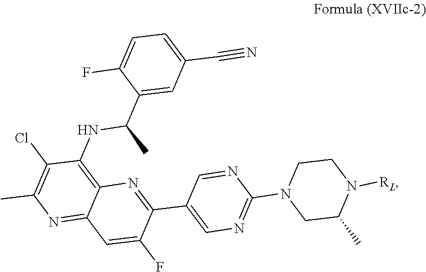
wherein R_{L} denotes attachment with L_{I}.

152. The bifunctional compound according to any one of claims 1 to 124, wherein T_{L} is according to formula XVIId

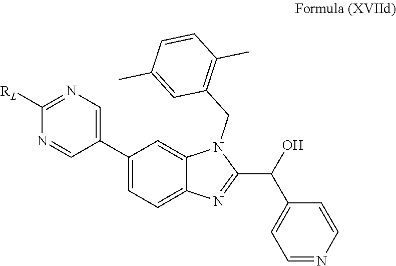

153. A compound selected from any one of the compounds TF001 to TF022 according to Table C in the section “List of compounds”.

154. The bifunctional compound according to any one of claims 1 to 124, wherein T_{L} comprises or consists of a protein, a peptide or a small molecule.

155. The bifunctional compound according to claim 154, wherein T_{L} comprises an antibody, an antibody fragment or a nanobody.

156. The bifunctional compound according to any one of claims 154 to 155, wherein T_{L} comprises a full length antibody.

157. The bifunctional compound according to any one of claims 154 to 156, wherein T_{L} comprises an antibody fragment.

158. The bifunctional compound according to claim 157, wherein T_{L} comprises an antibody light chain.

159. The bifunctional compound according to claim 157, wherein T_{L} comprises an antibody heavy chain.

160. The bifunctional compound according to claim 157, wherein T_{L} comprises a variable region of a heavy chain (V_{H}) of an antibody.

161. The bifunctional compound according to claim 157, wherein T_{L} comprises a variable region of a light chain (V_{L}) of an antibody.

162. The bifunctional compound according to any one of claims 1 or 154 to 155, wherein T_{L} comprises a fusion protein of two or more antibody fragments, such as two antibody fragments, for example three antibody fragments, for example 4 antibody fragments, such as 5 antibody fragments, such as 6 antibody fragments, such as 7 antibody fragments, such as 8 antibody fragments.

163. The bifunctional compound according to claim 162, wherein T_{L} comprises a fusion protein of two antibody fragments.

164. The bifunctional compound according to any one of claims 154 to 163, wherein T_{L} comprises a single chain antigen binding region (scAb).

165. The bifunctional compound according to any one of claims 154 to 163, wherein T_{L} comprises a single chain variable fragment (scFv).

166. The bifunctional compound according to any one of claims 154 to 156, wherein T_{L} comprises a nanobody.

167. The bifunctional compound according to any one of claims 1 or 154 to 155, wherein T_{L} is a protein, and the target protein is PCSK9, TNF-α or an antibody.

168. The bifunctional compound according to any one of claims 1 to 167, wherein the target protein is PCSK9.

169. The bifunctional compound according to any one of claims 1 to 167, wherein T_{L} is able to bind an antibody.

170. The bifunctional compound according to claim 169, wherein T_{L} is able to bind an IgG.

171. The bifunctional compound according to any one of claims to claims 169 to 170, wherein T_{L} is able to bind the light chain of an antibody.

172. The bifunctional compound according to any one of claims to claims 169 to 170, wherein T_{L} is able to bind the heavy chain of an antibody.

173. The bifunctional compound according to any one of claims to claims 169 to 170, wherein T_{L} is able to bind the constant region of the heavy chain (C_{H}) or the constant region of the light chain (C_{L}) of an antibody.

174. The bifunctional compound according to any one of claims 1 to 167, wherein the target protein is TNF-α.

175. The bifunctional compound according to any one of claims 1 to 124, wherein T comprises an antibody with binding specificity to PCSK-9 comprising:
a light chain region comprising SEQ ID NO.: 67 or an amino acid sequence having at least 70% sequence identity to SEQ ID NO.: 67, for example at least 80%, 85%, 90%, 95%, 98% or 99% sequence identity;
and/or
a heavy chain region comprising SEQ ID NO.: 66 or an amino acid sequence having at least 70% sequence identity to SEQ ID NO.: 66, for example at least 80%, 85%, 90%, 95%, 98% or 99% sequence identity;
or an antigen binding fragment thereof.

176. The bifunctional compound according to any one of claims 1 to 124, wherein T_{L} comprises or consists of an antibody with binding specificity to PCSK-9 comprising or consisting of:
a light chain region comprising SEQ ID NO.: 67
and/or
a heavy chain region comprising SEQ ID NO.: 66;
or an antigen binding fragment thereof.

177. The bifunctional compound according to any one of claims 1 to 124, wherein T_{L} comprises an antibody with binding specificity to TNF-α comprising: a light chain region comprising SEQ ID NO.: 69 or an amino acid sequence having at least 70% sequence identity to SEQ ID NO.: 69, for example at least 80%, 85%, 90%, 95%, 98% or 99% sequence identity;
and/or
a heavy chain region comprising SEQ ID NO.: 68.
or an amino acid sequence having at least 70% sequence identity to SEQ ID NO.: 68, for example at least 80%, 85%, 90%, 95%, 98% or 99% sequence identity;
or an antigen binding fragment thereof.

178. The bifunctional compound according to any one of claims 1 to 124, wherein T_{L} comprises or consists of an antibody with binding specificity to TNF-α comprising or consisting of:
a light chain region comprising SEQ ID NO.: 69
and/or
a heavy chain region comprising SEQ ID NO.: 68
or an antigen binding fragment thereof.

179. The bifunctional compound according to any one of claims 1 to 124, wherein T_{L} comprises a nanobody with binding specificity to the constant region of a second antibody comprising the following sequence:
(SEQ ID NO.: 70)
MGGTHHHHHHENLYFQGQVQLQESGGGLVQPGGSLRLSCAASGRTISR
YAMSWFRQAPGKEREFVAVARRSGDGAFYADSVQGRFTVSRDDAKNTV
YLQMNSLKPEDTAVYYCAIDSDTFYSGSYDYWGQGTQVTVSSE
or an amino acid sequence having at least 70% sequence identity to SEQ ID NO.: 70, for example at least 80%, 85%, 90%, 95%, 98% or 99% sequence identity.

180. The bifunctional compound according to any one of claims 1 to 124, wherein T_{L} comprises or consists of the following sequence:
(SEQ ID NO.: 70)
MGGTHHHHHHENLYFQGQVQLQESGGGLVQPGGSLRLSCAASGRTISR
YAMSWFRQAPGKEREFVAVARRSGDGAFYADSVQGRFTVSRDDAKNTV
YLQMNSLKPEDTAVYYCAIDSDTFYSGSYDYWGQGTQVTVSSE

181. The bifunctional compound according to anyone of the preceding claims, wherein the bifunctional compound is able to bind the extracellular target protein with a dissociation constant of less than 100 μM, such as less than 2 μM, such as less than 0.5 μM, preferably less than 0.1 μM.

182. The bifunctional compound according to any one of claims 1 to 16, wherein the bifunctional compound is according to any one of formulas XIVa, XIVb, XIVc and XIVd: 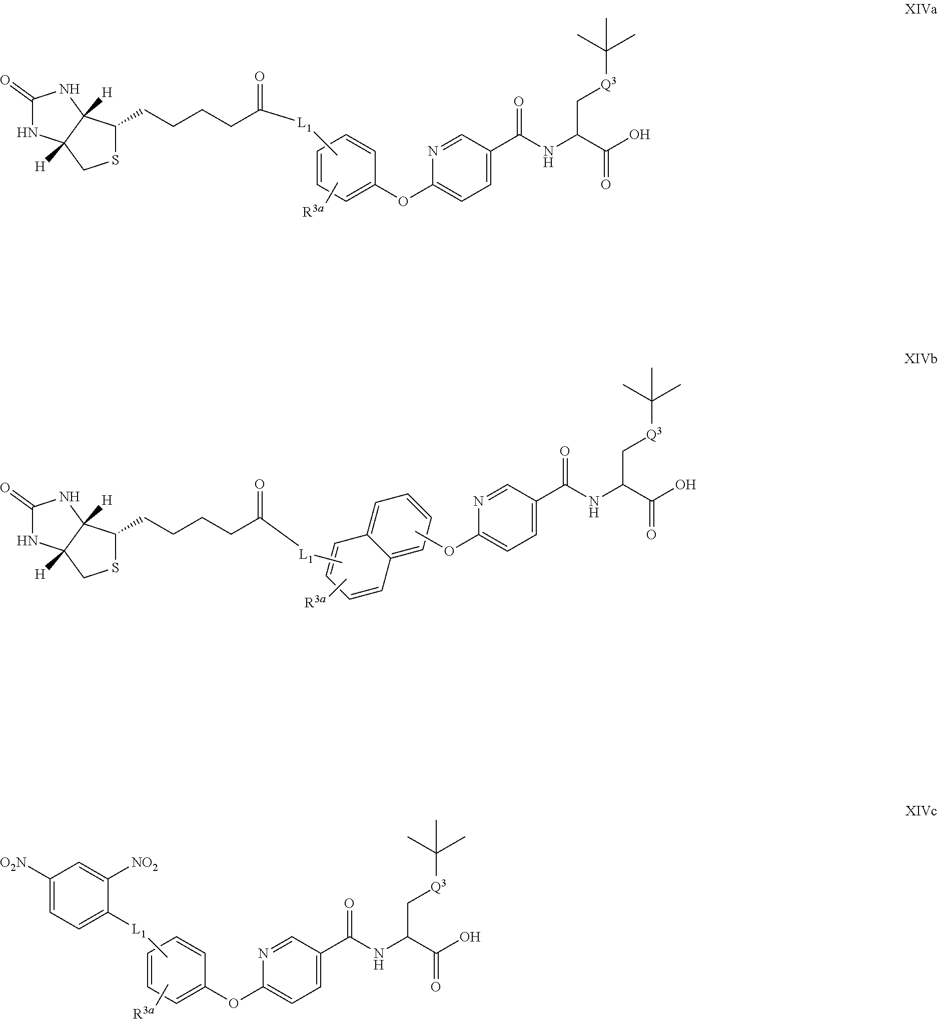 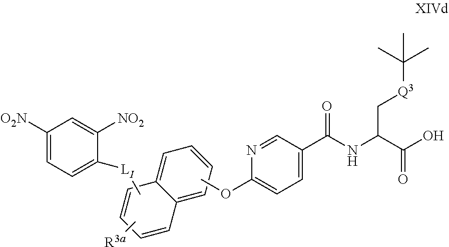
wherein
Q³ is a bond or —CH₂—;
R^{3a} is selected from the group consisting of H, halogen, alkoxy, —CF₃, and an optionally substituted C₁-₄ alkyl.

183. The bifunctional compound according to any one of claim 1 to 16, 138 to 151, or 181 wherein the bifunctional compound is according to any one of formulas XVIIIa, XVIIIb, XVIIIb-1, XVIIIc, XVIIId, XVIIId-1, XVIIIe, XVIIIf, XVIIIf-1, XVIIIg, XVIIIh and XVIIIh-1: 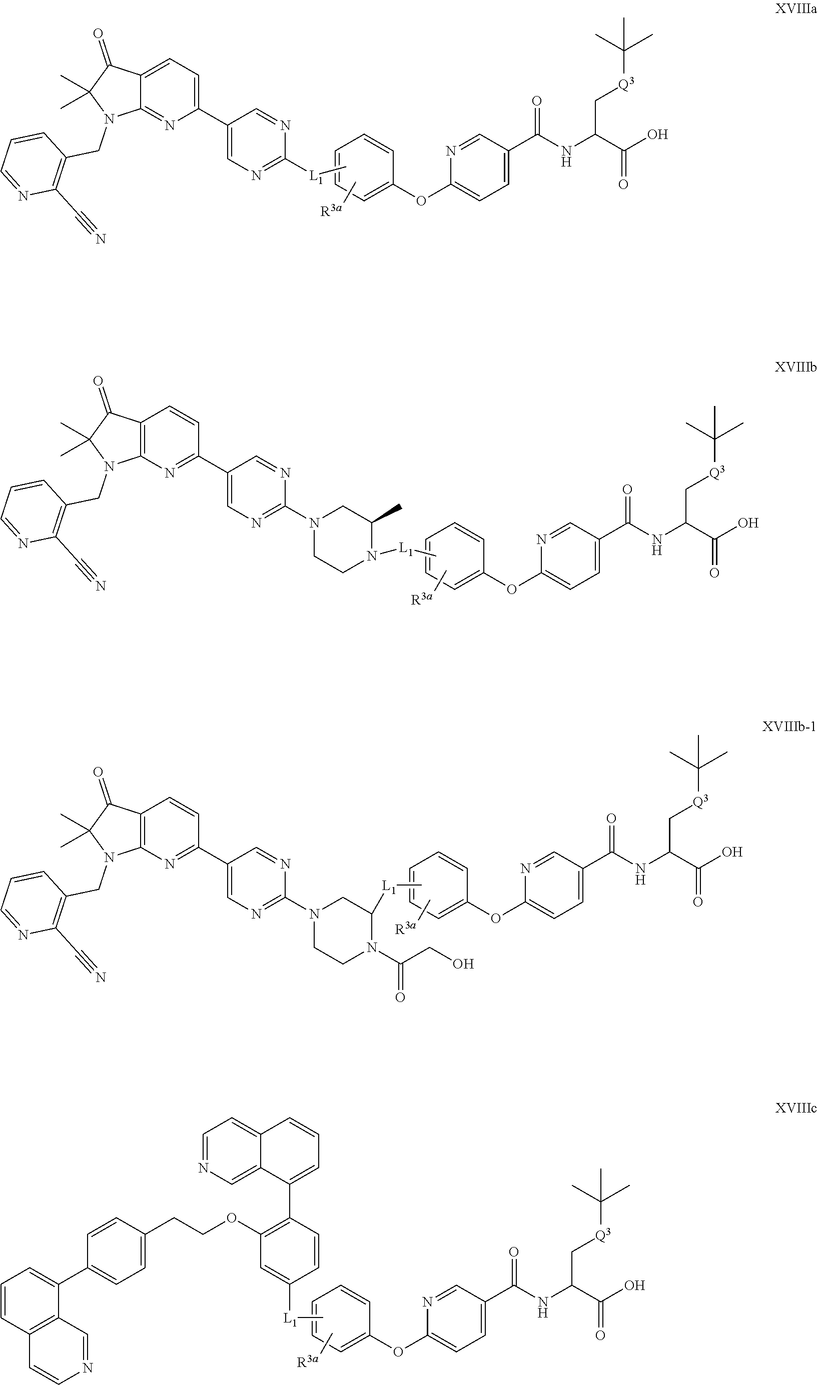 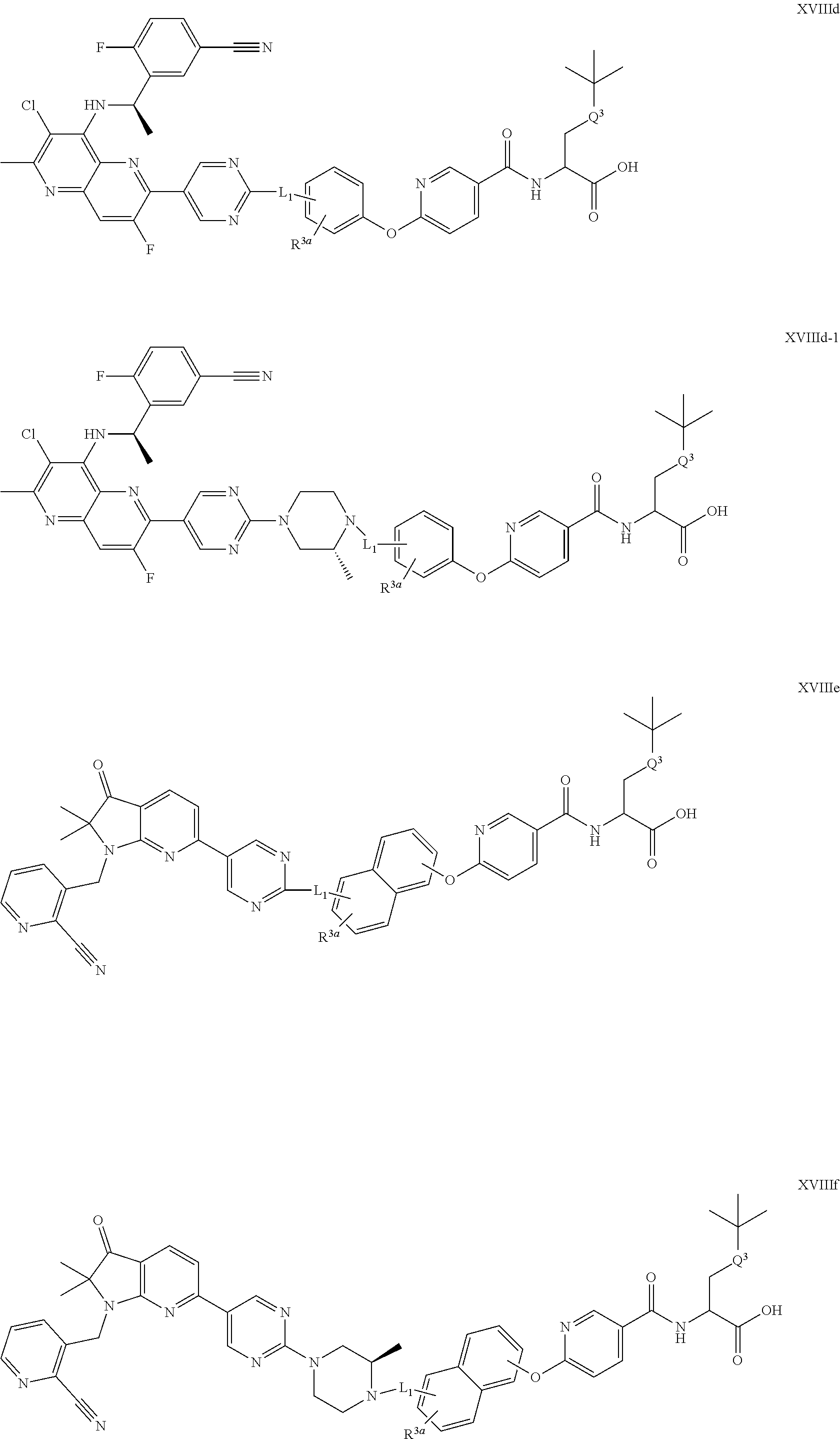 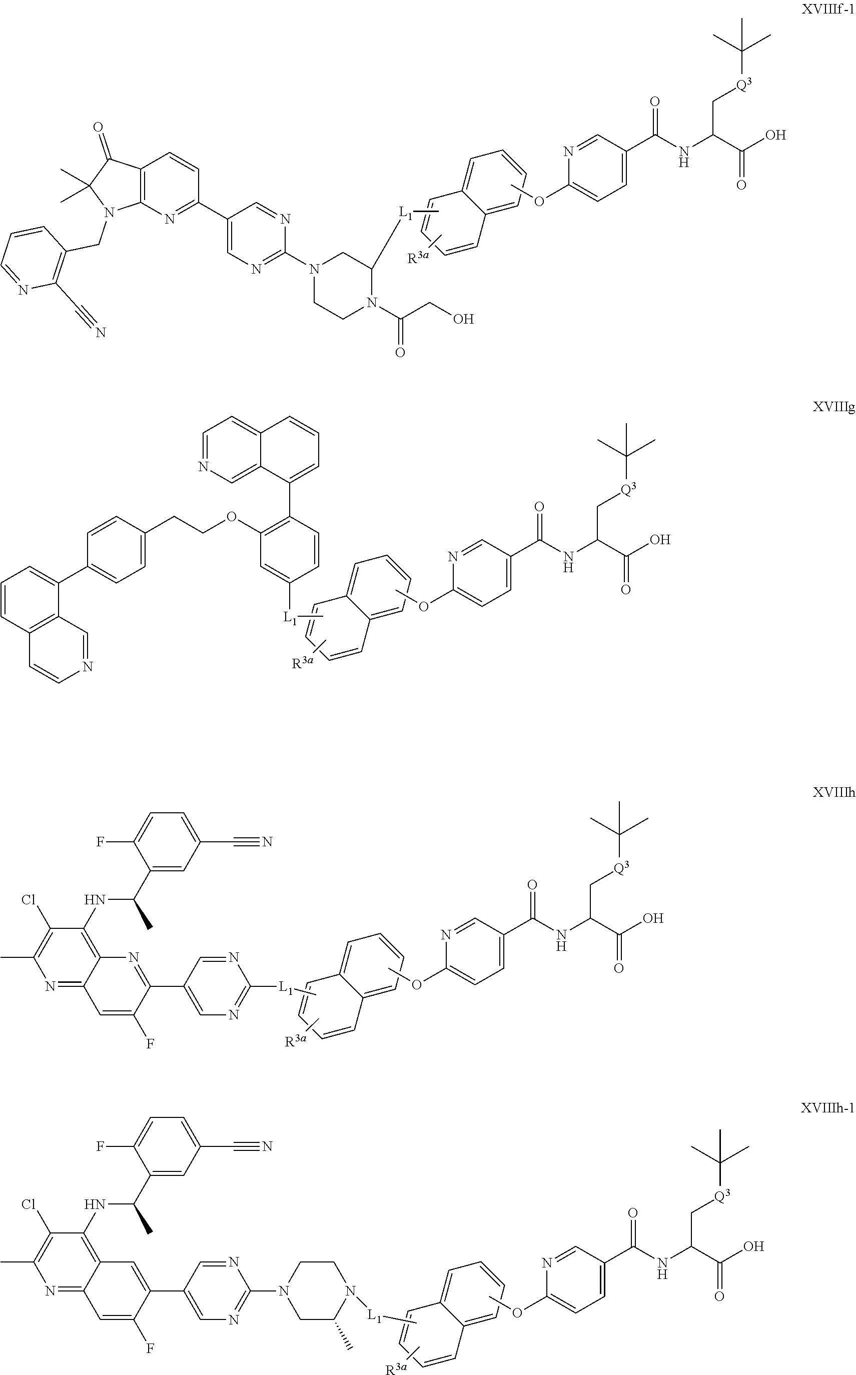
wherein
Q³ is a bond or —CH₂—;
R^{3a} is selected from the group consisting of H, halogen, alkoxy, —CF₃, and an optionally substituted C₁-₄ alkyl; and
L_{I} denotes the linker.

184. The bifunctional compound according to any one of claims 182 to 183, wherein R^{3a} is H.

185. The bifunctional compound according to any one of claims 182 to 183, wherein R^{3a} is halogen

186. The bifunctional compound according to any one of claims 182 to 183, wherein R^{3a} is —CF₃.

187. The bifunctional compound according to any one of claims 182 to 183, wherein R^{3a} is alkoxy.

188. The bifunctional compound according to any one of claims 182 to 183, wherein R^{3a} is an optionally substituted C₁-₄ alkyl.

189. The bifunctional compound according to any one of claims 182 to 183, wherein Q³ is a bond.

190. The bifunctional compound according to any one of claims 182 to 183, wherein Q³ is —CH₂—.

191. The bifunctional compound according to any one of claim 1 to 2, 17 to 19, 25, 138 to 151, or 181, wherein the bifunctional compound is according to any one of formulas D-III, D-IV, D-V and D-VI:

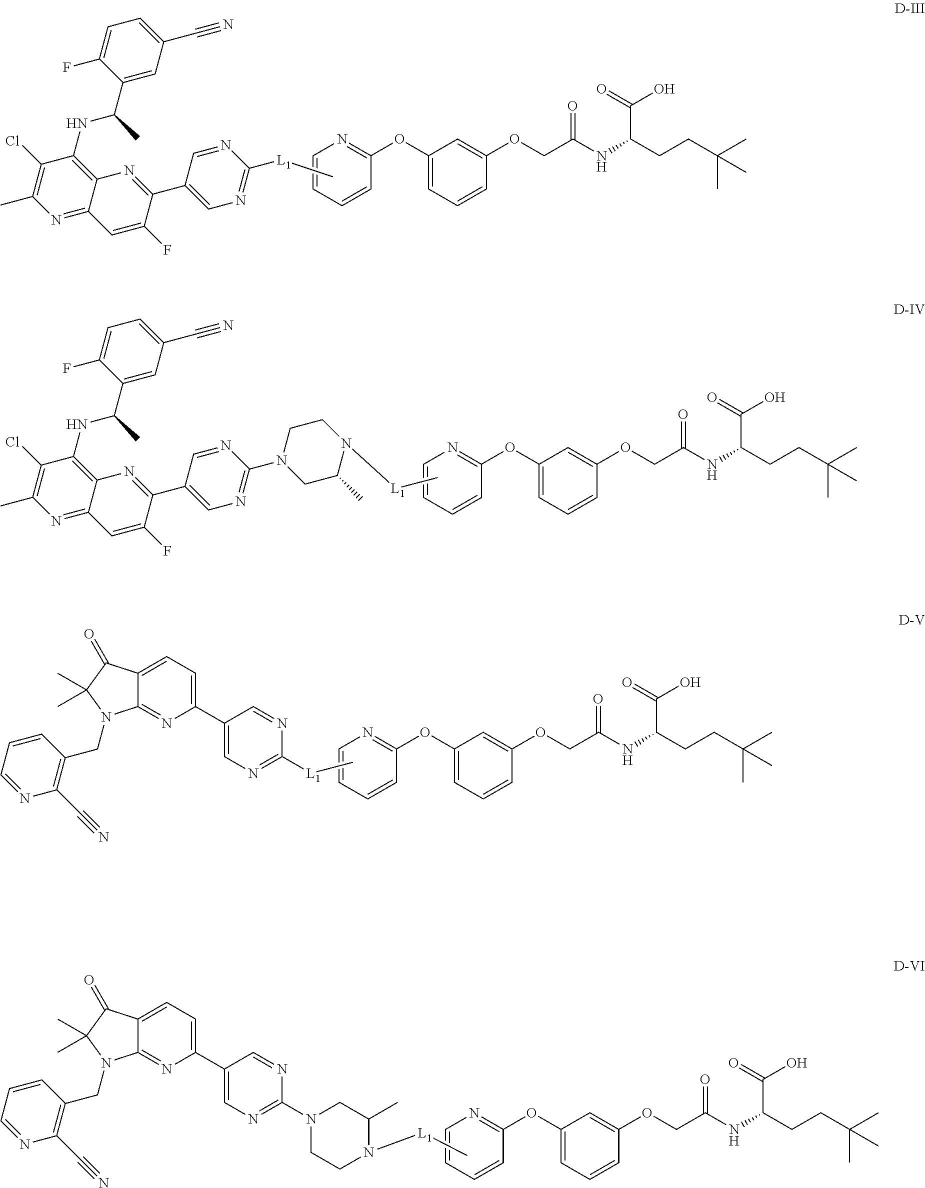

192. The bifunctional compound according to any one of claims 1 to 16, 19 to 25, 138 to 151, wherein the bifunctional compound is according to any one of formulas XVIIIm, XVIIIn, XVIIIn-1, XVIIIo, XVIIIp:

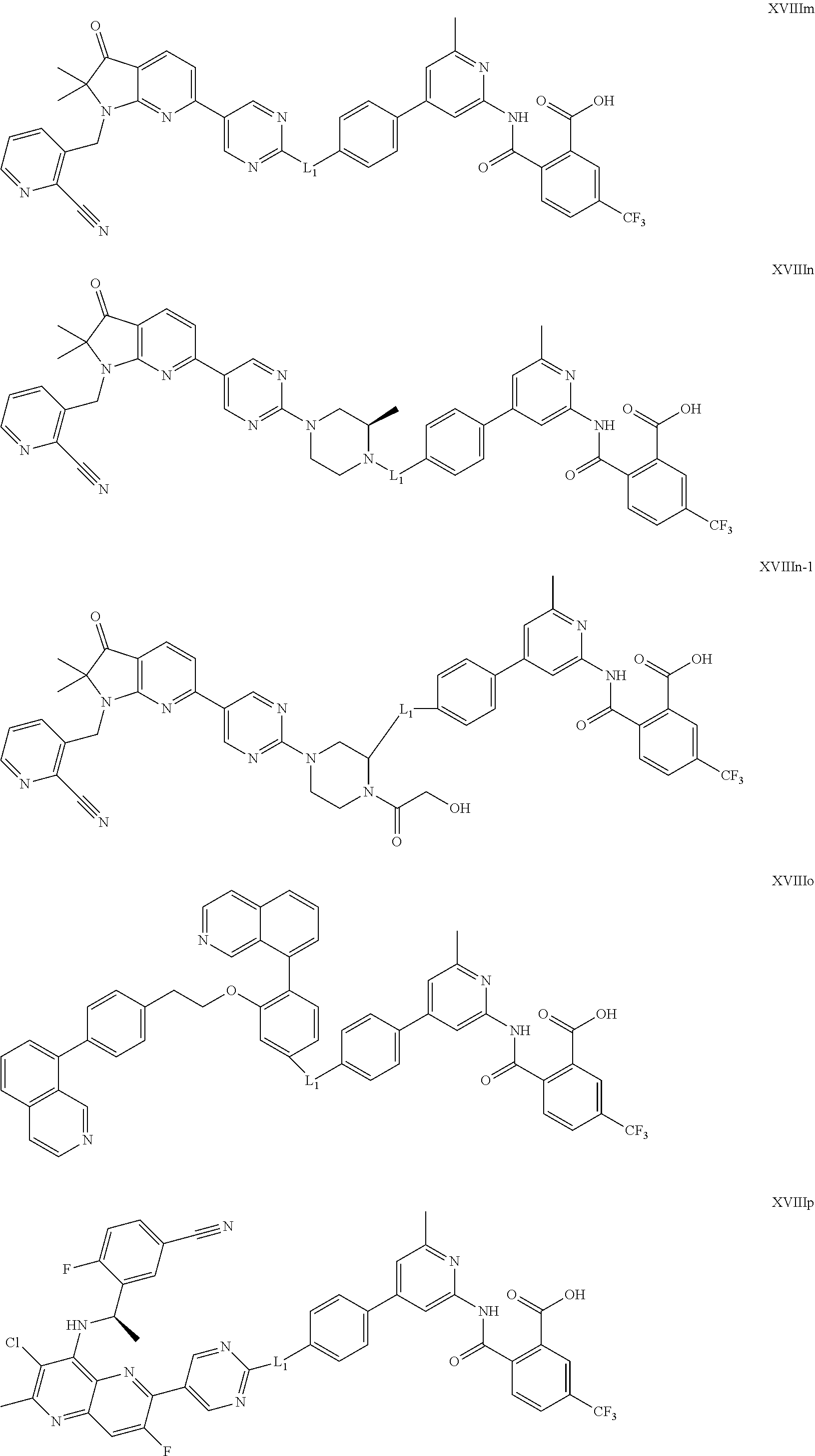

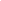

193. The bifunctional compound according to any one of claims 28 to 47, wherein the bifunctional compound is according to any one of formulas XVa, XVb, XVc and XVd: 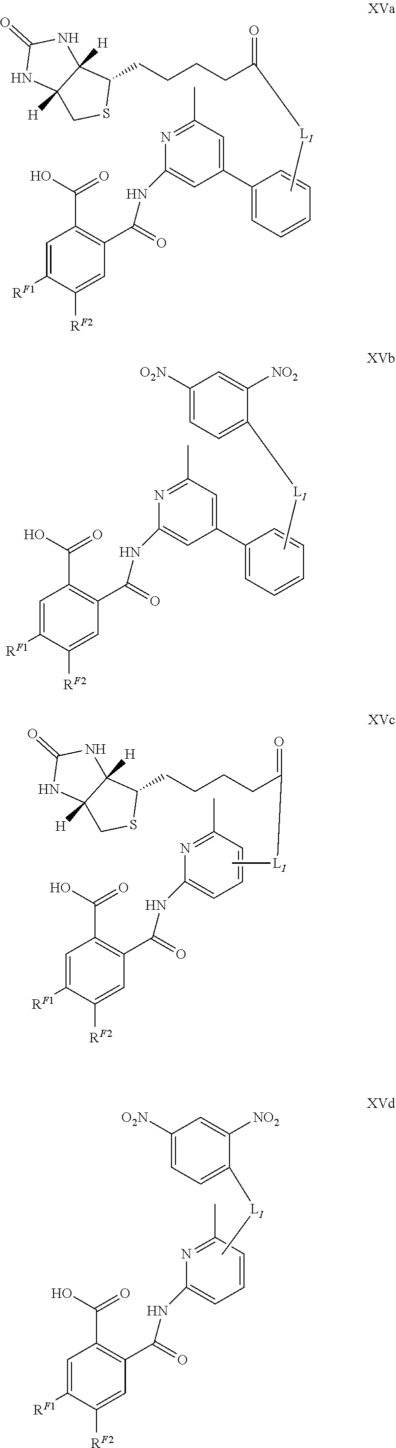
wherein,
R^{F1} and R^{F2} are independently selected from CF₃ or H, with the proviso that at least one of R^{F1} or R^{F2} is CF₃.

194. The bifunctional compound according to claim 193, wherein R^{F1} is —CF₃ and R^{F2} is H.

195. The bifunctional compound according to claim 1, wherein the bifunctional compound is selected from the group consisting of:
(SEQ ID NO.: 72)
X_{T}-X_{L}-RQLL-OH,
(SEQ ID NO.: 73)
X_{T}-X_{L}-FQLL-OH,
(SEQ ID NO.: 74)
X_{T}-X_{L}-KQLL-OH,
(SEQ ID NO.: 75)
X_{T}-X_{L}-PQLL-OH,
(SEQ ID NO.: 76)
X_{T}-X_{L}-PYIL-OH,
(SEQ ID NO.: 77)
X_{T}-X_{L}-RYLL-OH,
(SEQ ID NO.: 78)
X_{T}-X_{L}-FYLL-OH,
(SEQ ID NO.: 79)
X_{T}-X_{L}-YQLL-OH,
(SEQ ID NO.: 80)
X_{T}-X_{L}-LLQL-OH,
(SEQ ID NO.: 81)
X_{T}-X_{L}-FYIL-OH,
(SEQ ID NO.: 82)
X_{T}-X_{L}-RYIL-OH,
(SEQ ID NO.: 83)
X_{T}-X_{L}-PYLL-OH,
(SEQ ID NO.: 84)
X_{T}-X_{L}-PYYL-OH,
(SEQ ID NO.: 85)
X_{T}-X_{L}-PYFL-OH,
(SEQ ID NO.: 86)
X_{T}-X_{L}-RYYL-OH,
(SEQ ID NO.: 87)
X_{T}-X_{L}-RYFL-OH,
(SEQ ID NO.: 88)
X_{T}-X_{L}-RQFL-OH,
(SEQ ID NO.: 89)
X_{T}-X_{L}-RQYL-OH,
(SEQ ID NO.: 90)
X_{T}-X_{L}-LQLL-OH,
(SEQ ID NO.: 91)
X_{T}-X_{L}-HQLL-OH,
(SEQ ID NO.: 92)
X_{T}-X_{L}-IQLL-OH,
(SEQ ID NO.: 93)
X_{T}-X_{L}-FYYL-OH,
(SEQ ID NO.: 94)
X_{T}-X_{L}-PYMKLAPGELTIIL-OH,
(SEQ ID NO.: 95)
X_{T}-X_{L}-NEKLSQLQTYMI-OH,
(SEQ ID NO.: 96)
X_{T}-X_{L}-KDADLYTSRVMLSSQVP-OH,
(SEQ ID NO.: 99)
X_{T}-X_{L}-ITVDPRLFKKRRLRSPRVLFSTQPPR-OH,
(SEQ ID NO.: 100)
X_{T}-X_{L}-WSGPIGVSWGLRAAAAGGAFP-OH,
(SEQ ID NO.: 101)
X_{T}-X_{L}-WSGPIGVSWGLRAAAAGGAFPRGGRWRR-OH,
(SEQ ID NO.: 102)
X_{T}-X_{L}-GVSWGLR-OH,
(SEQ ID NO.: 103)
X_{T}-X_{L}-REAPRWDAPLRDPALRQLL-OH,
(SEQ ID NO.: 104)
X_{T}-X_{L}-REALRWDAPLRDPAPRQLL-OH,
(SEQ ID NO.: 105)
X_{T}-X_{L}-PYILKRQLYENKPRRPYIL-OH,
(SEQ ID NO.: 106)
X_{T}-X_{L}-LYENKPRRPYIL-OH,
(SEQ ID NO.: 107)
X_{T}-X_{L}-APLRDPAPRQLL-OH,
(SEQ ID NO.: 108)
X_{T}-X_{L}-REAPRWDAPLRDPALFQLL-OH,
(SEQ ID NO.: 109)
X_{T}-X_{L}-REAPRWDAPLRDPALRYLL-OH,
(SEQ ID NO.: 110)
X_{T}-X_{L}-REAPRWDAPLRDPALRQYL-OH,
(SEQ ID NO.: 111)
X_{T}-X_{L}-REAPRWDAPLRDPALRYYL-OH,
(SEQ ID NO.: 112)
X_{T}-X_{L}-TGGFM-OH,
and
(SEQ ID NO.: 113)
X_{T}-X_{L}-YGGFL-OH;
wherein X_{L} is L_{I} and X_{T} is selected from the group consisting of:
a) a group comprising biotin;
b) a group comprising dinitrophenol;
c) an antibody with binding specificity to PCSK-9 comprising or consisting of:
a light chain region comprising SEQ ID NO.: 67
and/or
a heavy chain region comprising SEQ ID NO.: 66
or an antigen binding fragment thereof.
d) an antibody with binding specificity to TNF-α comprising or consisting of:
a light chain region comprising SEQ ID NO.: 69
and/or
a heavy chain region comprising SEQ ID NO.: 68
or an antigen binding fragment thereof.

196. The bifunctional compound according to claim 1, wherein the bifunctional compound is:
(SEQ ID NO.: 116)
Biotin-X_{L}-X₅-X₁X₂X₃X₄
wherein,
X₅ is an optional amino acid residue or peptide comprising 2 to 30 amino acid residues or a bond,
X₁ is R, P, F, Y, L, K, G or H;
X₂ is Q, Y, L, E or G;
X₃ is Y, F, L, I, Q, E or N;
X₄ is M, K, L or a conservative substitution of L;
wherein X_{L} is L_{I}.

197. The bifunctional compound according to claim 196, wherein X₅ is a bond.

198. The bifunctional compound according to claim 196, wherein X₅ is
(SEQ ID NO.: 21)
APLRDPAP.

199. The bifunctional compound according to claim 196, wherein X₅ is
(SEQ ID NO.: 17)
REAPRWDAPLRDPAL.

200. The bifunctional compound according to claim 196, wherein X₅ is
(SEQ ID NO.: 18)
REALRWDAPLRDPAP.

201. The bifunctional compound according to claim 196, wherein
X₁ is selected from Y, F or R,
X₂ is selected from Q or Y,
X₃ is L,
X₄ is L.

202. The bifunctional compound according to claim 196, wherein X₁ is F or R.

203. The bifunctional compound according to claim 196, wherein X₂ is Q.

204. The bifunctional compound according to claim 196, wherein X₃ is Q.

205. The bifunctional compound according to claim 196, wherein X₄ is L.

206. The bifunctional compound according to any one of claims 196 to 205 wherein the bifunctional compound is according to any one of selected from:
(SEQ ID NO.: 117)
Biotin-X_{L}- RQLL-OH
(SEQ ID NO.: 118)
Biotin-X_{L}-FQLL-OH
(SEQ ID NO.: 119)
Biotin-X_{L}-RYLL-OH
(SEQ ID NO.: 120)
Biotin-X_{L}-FYLL-OH
(SEQ ID NO.: 121)
Biotin-X_{L}-YQLL-OH
(SEQ ID NO.: 122)
Biotin-X_{L}-REAPRWDAPLRDPALRQLL-OH
(SEQ ID NO.: 123)
Biotin-X_{L}-REALRWDAPLRDPAPRQLL-OH
(SEQ ID NO.: 124)
Biotin-X_{L}-REAPRWDAPLRDPALFQLL-OH
(SEQ ID NO.: 125)
Biotin-X_{L}-REAPRWDAPLRDPALRYLL-OH
(SEQ ID NO.: 126)
Biotin-X_{L}-REAPRWDAPLRDPALRQYL-OH
(SEQ ID NO.: 127)
Biotin-X_{L}-REAPRWDAPLRDPALRYYL-OH
(SEQ ID NO.: 128)
Biotin-X_{L}-APLRDPAPRQLL-OH
(SEQ ID NO.: 129)
Biotin-X_{L}-PYILKRQLYENKPRRPYIL-OH;
and
(SEQ ID NO.: 130)
Biotin-X_{L}-LYENKPRRPYIL-OH
wherein X_{L} is L_{I}.

207. The bifunctional compound according to claim 1 wherein T_{L} is a monoclonal antibody conjugated to at least 1 unit of -L_{I}-S_{L}, for example conjugated to 1 unit of -L_{I}-S_{L}, for example conjugated to 2 units of -L_{I}-S_{L}, for example conjugated to 3 unit of -L_{I}-S_{L}, for example conjugated to 4 units of -L_{I}-S_{L}.

208. The bifunctional compound according to claim 207, wherein T_{L} is the monoclonal antibody alirocumab (antibody with heavy chain of SEQ ID NO.: 66 and light chain of SEQ ID NO.: 67).

209. The bifunctional compound according to claim 207, wherein T_{L} is the monoclonal antibody adalimumab (antibody with heavy chain of SEQ ID NO.: 68 and light chain of SEQ ID NO.: 69).

210. The bifunctional compound according to any one of claims 207 to 209, wherein S_{L} is a peptide comprising a sequence at the C-terminus selected from the group consisting of:
(SEQ ID NO.: 22)
RQLL-OH
(SEQ ID NO.: 23)
FQLL-OH
(SEQ ID NO.: 27)
RYLL-OH
(SEQ ID NO.: 28)
FYYL-OH
(SEQ ID NO.: 29)
YQLL-OH
and
(SEQ ID NO.: 55)
PYILKRQLYENKPRRPYIL-OH;
wherein L_{I} is attached at the N-terminus.

211. The bifunctional compound according to any one of claims 207 to 209, wherein S_{L} is a peptide comprising at the C-terminus RQLL-OH (SEQ ID NO.: 22).

212. The bifunctional compound according to any one of claims 207 to 209, wherein S_{L} is a peptide comprising at the C-terminus FQLL-OH (SEQ ID NO.: 23).

213. The bifunctional compound according to any of the preceding claims, wherein L_{I} has the following structure: 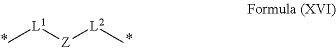
wherein
* denotes the point of attachment to either T_{L} or S_{L};
L₁ and L₂ are each independently selected from the group consisting of a bond, —C(H₂)—, —O—, —N(H)—, a functional group selected from carbonyl, ester, amide, carbamate, thiourea, urea, sulphonamide and triazole; and a C₁-C₃ hydrocarbon chain wherein one or more methylene groups are individually and optionally replaced with a carbonyl, ester, amide, carbamate, thiourea, urea sulphonamide and triazole;
Z is selected from the group consisting of: a bivalent, saturated or unsaturated, straight or branched, C₁-C₃₀ hydrocarbon chain wherein one or more methylene groups are individually and optionally replaced by one or more of the groups selected from: —O—, —N(H)—, —N(R^{L1})—, —OC(═O)—, —C(═O)O—, —C(═O)—, —N(H)C(═O)—, —N(R^{L1})C(═O)—, —C(═O)N(H)—, —NHC(O)NH—, —NHC(O)O—C(═O)N(R^{L1})—, —S—, —S(═O)—, —S(═O)₂—, —N(R^{L1})S(═O)₂—, —S(═O)₂N(R^{L1})—; an optionally substituted aromatic group; an optionally substituted carbocycle; an optionally substituted heterocycle; an optionally substituted aromatic heterocycle; 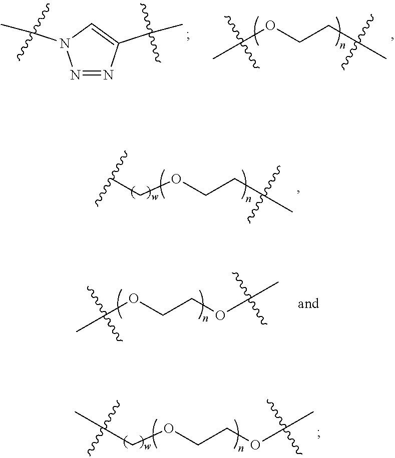
R^{L1} is selected from the group consisting of C₁₋₅ alkyl;
n and w each individually integers from 1 to 9.

214. The bifunctional compound according to any of the preceding claims, wherein L_{I} has the following structure: 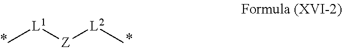
wherein
* denotes the point of attachment to either T_{L} or S_{L},
L₁ and L₂ are each independently selected from the group consisting of —C(H₂)—, —O—, —N(H)—, and an amide;
Z is a bivalent, saturated or unsaturated, straight or branched, C₁-C₃₀ hydrocarbon chain wherein one or more methylene groups are individually and optionally replaced by one or more of the groups selected
from: —O—, —N(H), —N(R^{L1})—, —N(H)C(═O)—, —N(R)C(═O)—, —C(═O)N(H)—, —C(═O)N(R^{L1})—, 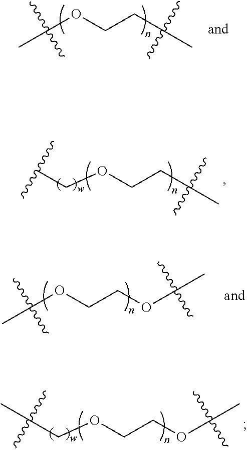
R^{L1} is selected from the group consisting of C₁₋₅ alkyl;
n and w each individually integers from 1 to 9.

215. The bifunctional compound according any one of claims 213 to 214, wherein the C₁-C₃₀ hydrocarbon chain is C₅-C₃₀ hydrocarbon chain, such as a C₈-C₃₀ hydrocarbon chain, such as a C₁₀-C₃₀ hydrocarbon chain, such as a C₁₂-C₃₀ hydrocarbon chain.

216. The bifunctional compound according any one of claims 213 to 214, wherein the C₁-C₃₀ hydrocarbon chain is a C₁₀-C₂₅ hydrocarbon chain, such as a C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ or C₂₅ hydrocarbon chain.

217. The bifunctional compound according any one of claims 213 to 214, wherein the C₁-C₃₀ hydrocarbon chain is a C₁₄-C₂₀ hydrocarbon chain.

218. The bifunctional compound according any one of claims 213 to 214, the C₁-C₃₀ hydrocarbon chain is a C₇-C₁₃ hydrocarbon chain.

219. The bifunctional compound according any one of claims 213 to 214, wherein Z is a C₁-C₃₀ hydrocarbon chain wherein one or more methylene groups are individually and optionally replaced by one or more of the groups selected from: —O—, —N(H), —N(H)—C(═O)—, —C(═O)—N(H)—, a triazole, an optionally substituted carbocycle; an optionally substituted heterocycle, and —CH₂—CH₂—O—.

220. The bifunctional compound according any one of claims 213 to 214, wherein Z comprises a C₁-C₃₀ hydrocarbon chain, such as a C₁-C₂₀, such as a C₁-C₁₅, such as a C₁-C₁₀ hydrocarbon chain; wherein one or more methylene groups are individually and optionally replaced by one or more of the groups selected from: —O—, —N(H)—, —N(R^{L1})—, —OC(═O)—, —C(═O)O—, —C(═O)—, —N(H)C(═O)—, —, —NHC(═O)O—, —N(R^{L1})C(═O)—, —C(═O)N(H)—, —C(═O)N(R^{L1})—, —S—, —S(═O)—, —S(═O)₂—, —N(R^{L1})S(═O)₂—, —S(═O)₂N(R^{L1})—, —CH₂—CH₂—O—, an optionally substituted carbocycle; an optionally substituted heterocycle, and a triazole; and R^{L1} is C₁₋₅ alkyl.

221. The bifunctional compound according any one of claims 213 to 220, wherein one or more methylene groups, such as 1, 2, 3, 4, 5, 6, 7, 8, 9 or 10 methylene groups of the hydrocarbon chain in Z are individually and optionally replaced by one or more of the groups selected from —O—, —N(H)—, —N(R^{L1})—, —OC(═O)—, —C(═O)O—, —C(═O)—, —N(H)C(═O)—, —N(R^{L1})C(═O)—, —C(═O)N(H)—, —C(═O)N(R^{L1})—, —S—, —S(═O)—, —S(═O)₂—, —N(R^{L1})S(═O)₂—, —S(═O)₂N(R^{L1})—, —CH₂—CH₂—O—, an optionally substituted carbocycle; an optionally substituted heterocycle and a triazole; and
R^{L1} is C₁₋₅ alkyl.

222. The bifunctional compound according to any one of claims 213 to 220, wherein Z comprises one or more groups —NH—SO²— groups.

223. The bifunctional compound according to any one of claims 213 to 221, wherein Z comprises one or more triazole groups.

224. The bifunctional compound according to any one of claims 213 to 223, wherein Z comprises one or more groups selected from: an optionally substituted carbocycle group(s) and an optionally substituted heterocycle group(s).

225. The bifunctional compound according to any one of claims 213 to 224, wherein Z comprises two groups each individually selected from: a triazole, an optionally substituted carbocycle group(s) and an optionally substituted heterocycle group(s).

226. The bifunctional compound according to any one of claims 213 to 224, wherein Z, comprises three groups each individually selected from: a triazole, an optionally substituted carbocycle group(s) and an optionally substituted heterocycle group(s).

227. The bifunctional compound according to any one of claims 213 to 226, wherein the carbocyle is according to 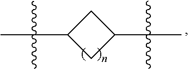
wherein n is an integer selected from 0, 1, 2 or 3.

228. The bifunctional compound according to any one of claims 213 to 227, wherein Z comprises one or more heterocycle groups.

229. The bifunctional compound according to any one of claims 213 to 228, wherein the heterocycle group may be an optionally substituted 3 to 6 membered ring wherein one or two carbon atoms of the ring have been replaced by N.

230. The bifunctional compound according to any one of claims 213 to 229, wherein the heterocycle group is according to 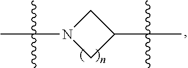
wherein n is an integer selected from 0, 1, 2 or 3.

231. The bifunctional compound according to any one of claims 213 to 230, wherein Z comprises one or more groups, each individually selected from the group consisting of shown in Table Z in the section “Linkers”.

232. The bifunctional compound according to any one of claims 213 to 231, wherein Z comprises two groups each individually selected from the groups shown in Table Z in the section “Linkers”.

233. The bifunctional compound according to any one of claims 213 to 232, wherein Z comprises three groups each individually selected from the groups shown Table Z in the section “Linkers”.

234. The bifunctional compound according to any one of claims 213 to 233, wherein Z comprises one or more groups each individually selected from the groups shown in table Z—I in the section “Linkers”.

235. The bifunctional compound according to any one of claims 213 to 234, wherein the triazole group is

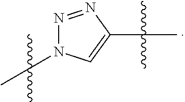 [SMILES: *c1cn(*)nn1]

236. The bifunctional compound according to any one of claims 213 to 235, wherein Z comprises 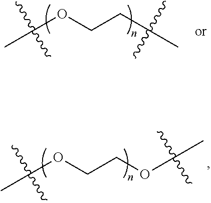
wherein n is an integer from 1 to 10.

237. The bifunctional compound according to any one of claims 213 to 236, wherein Z comprises 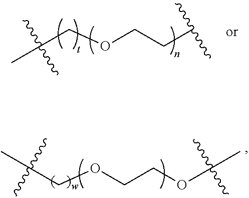
wherein n is an integer from 1 to 10 and t or w is an integer from 1 to 20.

238. The bifunctional compound according to any one of claims 213 to 237, wherein Z comprises 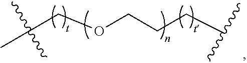
wherein n is an integer from 1 to 10 and each of t and t′ is individually an integer from 1 to 20.

239. The bifunctional compound according to any one of claims 213 to 238, wherein Z comprises 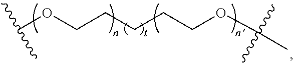
wherein each of n and n′ is individually an integer from 1 to 10 and t is an integer from 1 to 20.

240. The bifunctional compound according to any one of claims 213 to 239 wherein L¹ or L² are an amide group.

241. The bifunctional compound according to any one of claims 213 to 239, wherein L¹ and L² are an amide group.

242. The bifunctional compound according to any one of claims 213 to 239, wherein L¹ or L² are a carbonyl group.

243. The bifunctional compound according to any one of claims 213 to 239, wherein L¹ and L² are a carbonyl group.

244. The bifunctional compound according to any one of claims 213 to 239, wherein L¹ or L² are an ester group.

245. The bifunctional compound according to any one of 213 to 239, wherein L¹ and L² are an ester group

246. The bifunctional compound according to any one of claims 213 to 239, wherein L¹ or L² are a carbamate group.

247. The bifunctional compound according to any one of claims 213 to 239, wherein L¹ and L² are a carbamate group.

248. The bifunctional compound according to any one of claims 213 to 239, wherein L¹ and L² are an urea group.

249. The bifunctional compound according to any one of claims 213 to 239, wherein L¹ or L² are an urea group.

250. The bifunctional compound according to any one of claims 213 to 239, wherein L¹ or L² are —NH—S(═O)₂—.

251. The bifunctional compound according to any one of claims 213 to 239, wherein L¹ and L² are —NH—S(═O)₂—.

252. The bifunctional compound according to any one of claims 213 to 239, wherein L¹ or L² are a triazole group.

253. The bifunctional compound according to any one of claims 213 to 239, wherein L¹ and L² are a triazole group.

254. The bifunctional compound according to any one of claims 213 to 239, wherein L¹ or L² are —O—.

255. The bifunctional compound according to any one of claims 213 to 239, wherein L¹ and L² are —O—.

256. The bifunctional compound according to any one of claims 213 to 239, wherein L¹ or L² are —NH—.

257. The bifunctional compound according to any one of claims 213 to 239, wherein L¹ and L² are —NH—.

258. The bifunctional compound according to any one of claims 213 to 239, wherein L¹ or L² are a —S(═O)₂—.

259. The bifunctional compound according to any one of claims 213 to 239, wherein L¹ and L² are a-S(═O) 2-.

260. The bifunctional compound according to any one of claims 213 to 239, wherein L¹ or L² are

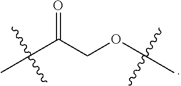 [SMILES: *OCC(*)=O]

261. The bifunctional compound according to any one of claims 213 to 239, wherein L¹ and L² are

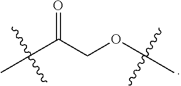 [SMILES: *OCC(*)=O]

262. The bifunctional compound according to any one of claims 213 to 239, wherein L¹ or L² are

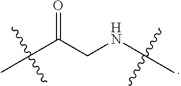 [SMILES: *NCC(*)=O]

263. The bifunctional compound according to any one of claims 213 to 239, wherein L¹ and L² are

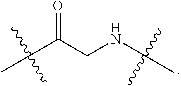 [SMILES: *NCC(*)=O]

264. The bifunctional compound according to any one of claims 213 to 239, wherein L¹ and L² are different groups.

265. The bifunctional compound according to any one of claims 213 to 239, wherein L¹ and/or L² are

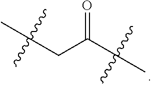 [SMILES: *CC(*)=O]

266. The bifunctional compound according to any one of claims 213 to 239, wherein L¹ and/or L² are

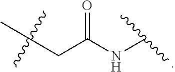 [SMILES: *CC(=O)N*]

267. The bifunctional compound according to any one of claims 213 to 239, wherein L¹ and/or L² are 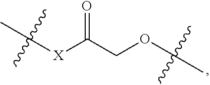
wherein X is an atom selected from N or O.

268. The bifunctional compound according to any one of claims 213 to 239, wherein L¹ and/or L² are 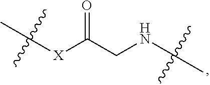
wherein X is an atom selected from N or O.

269. The bifunctional compound according to any one of claims 213 to 239, wherein L¹ and L² are identical.

270. The bifunctional compound according to any one of claims 213 to 239, wherein L¹ is a bond.

271. The bifunctional compound according to any one of claims 213 to 239, wherein L² is a bond.

272. The bifunctional compound according to any one of claims 213 to 239, wherein L¹ and L² are bonds.

273. The bifunctional compound according to any of the preceding claims, wherein L_{I} is selected from the group consisting of: 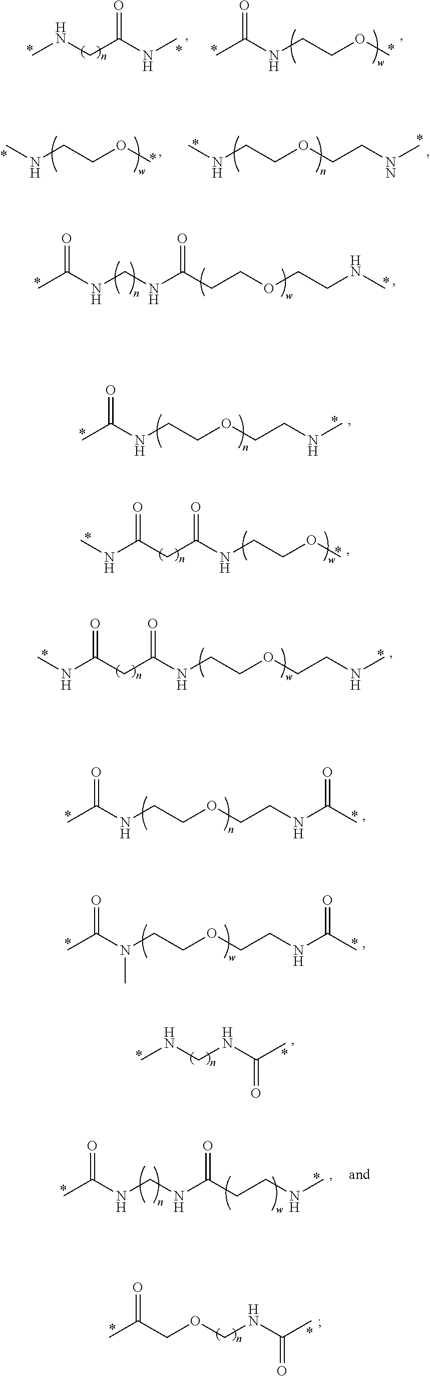
n and w are each individually integers from 1 to 9 and
* denotes the attachment to either T_{L} or S_{L}.

274. The bifunctional compound according to any of the preceding claims, wherein L_{I} is selected from any one of the formulas (XVIa) to Formula (XVIae) in Table Z-II of the section “Linkers”; wherein R^{L} denotes the point of attachment to S_{L} and R^{T} denotes the point of attachment to T_{L}.

275. The bifunctional compound according to any of the preceding claims, wherein L_{I} is selected from any one of the formulas XVIaf to XVIbw in Table Z-III of the section “Linkers” wherein * denotes the attachment either with S_{L} or T_{L}.

276. The bifunctional compound according to claim 1, wherein L_{I} is a peptide.

277. The bifunctional compound according to claim 276, wherein L_{I} is a peptide.

278. The bifunctional compound according to any one of claims 276 to 277, wherein L_{I} is a peptide of a length between 1 to 30 amino acids.

279. The bifunctional compound according to any one of claims 276 to 278, wherein L_{I} is a peptide of a length between 3 to 20 amino acids.

280. The bifunctional compound according to any one of claims 276 to 279, wherein L_{I} is a peptide of a length between 3 to 20 amino acids consisting of any combination of glycine, serine and cysteine.

281. The bifunctional compound according to any one of claims 276 to 280, wherein L_{I} is a peptide of a length between 3 to 20 amino acids consisting of any combination of glycine and serine.

282. The bifunctional compound according to any one of claims 276 to 281, wherein L_{I} is a peptide selected from any one of the group consisting of:
(SEQ ID NO.: 131)
GGSGGGGGGGGSGG
(SEQ ID NO.: 132)
GGSGGGG
(SEQ ID NO.: 133)
GGGGSGGGGSGGGGSGG
(SEQ ID NO.: 134)
GGSGGGGSGGGGS
(SEQ ID NO.: 135)
GGSGGGGSGGG
(SEQ ID NO.: 136)
GGSGGGGSG
(SEQ ID NO.: 137)
GGSGGGG
(SEQ ID NO.: 138)
GGSGG
(SEQ ID NO.: 139)
GGS
(SEQ ID NO.: 140)
CGGSGGGGSGGGGSGG.

283. The bifunctional compound according to any of claims 276 to 282, wherein L_{I} is connected to S_{L} via the C-terminus of L_{I} and L_{I} is connected to T_{L} via the N-terminus of L_{I}.

284. The bifunctional compound according to claims 1 to 16, wherein S_{L} is according to formula IIIai; 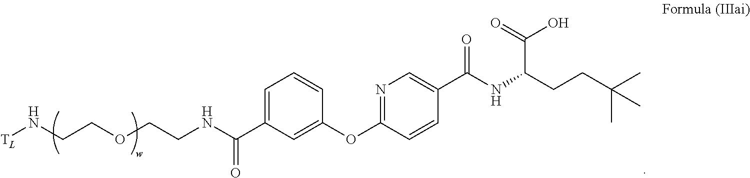
wherein w is an integer between 1 and 9.

285. The bifunctional compound according to any one of claims 1 to 47, 138 to 151 or 213 to 275, wherein the bifunctional compound is selected from any one of compounds BF001 to BF028 as shown in Table A in the section “List of Compounds”.

286. The bifunctional compound according to any one of claims 1 to 47, 138 to 151 or 213 to 275, wherein the bifunctional compound is selected from any one of compounds BF030 to BF149 as shown in Table A in the section “List of Compounds”.

287. The bifunctional compound according to any one of claims 1 to 47, 138 to 151 or 213 to 275, wherein the bifunctional compound has:
a. S_{L} selected from any one from the group consisting of formulas any one of formulas IIIi, IIIj, IIIk, IIIm, IIIn, and IIIo; or selected from formula D-I as described in the section “Binding of Sortilin” above; and
b. is able to bind the target molecule with a dissociation constant (K_{D}) of less than 50 μM, such as less than 40 μM, such as less than 30 μM, such as less than 20 μM, such as less than 10 μM, such as less than 5 μM, such as less than 4 μM, such as less than 3 μM, such as less than 2 μM, such as less than 1 μM, such as less than 0.8 μM, such as less than 0.6 μM, such as less than 0.5 μM, such as less than 0.4 μM, such as less than 0.3 μM, such as less than 0.2 μM, such as less than 0.1 μM.

288. The bifunctional compound according to any one of claims 1 to 47, 59 to 60, 138 to 151 or 213 to 275, wherein the bifunctional compound has:
a. S_{L} selected from any one from the group consisting of formulas any one of formulas IIIi, IIIj, IIIk, IIIm, IIIn, and IIIo; or selected from formula D-I as described in the section “Binding of Sortilin”; and
b. L_{I} selected from any one of formulas any one of formulas XVIaf to XVIbw in Table Z-III in the section “Linkers” above; and
c. T_{L} selected from any one of formulas XVIIa-3, XVIIa-4, XVIIc-1 and XVIIc-2 as described in the section “Targeting warhead”.

289. The bifunctional compound according to claim 1 or to any one of claims 61 to 118 or claims 276 to 282, wherein the bifunctional compound is selected from the group consisting of:
(SEQ ID NO.: 141)
Biotin-GGSGGGGSGRQLL-OH
(SEQ ID NO.: 142)
Biotin-GGSGGGGSGGGGSRQLL-OH
(SEQ ID NO.: 143)
Biotin-GGSGGGGFQLL-OH
(SEQ ID NO.: 144)
Biotin-GGSGGFQLL-OH
(SEQ ID NO.: 145)
Biotin-GGSGGGGSGFQLL-OH
(SEQ ID NO.: 146)
Biotin-GGSGGGGSGGGGSFQLL-OH
(SEQ ID NO.: 147)
Biotin-GGSGGGGSGGGGSGGRQLL-OH
(SEQ ID NO.: 148)
Biotin-GGSGGGGSGGGFQLL-OH
(SEQ ID NO.: 149)
Biotin-GGSGGGGSGGGRQLL-OH
(SEQ ID NO.: 150)
Biotin-GGSGGGGRQLL-OH
(SEQ ID NO.: 151)
Biotin-GGSGGRQLL-OH
(SEQ ID NO.: 152)
Biotin-GGSRQLL-OH
(SEQ ID NO.: 153)
Biotin-GGRQLL-OH
(SEQ ID NO.: 154)
Biotin-RQLL-OH
(SEQ ID NO.: 155)
Biotin-GGSGGGGSGGGGSGGFQLL-OH
(SEQ ID NO.: 189)
Biotin-REAPRWDAPLRDPALFQLL-OH,
and
(SEQ ID NO.: 191)
Biotin-REAPRWDAPLRDPALRQLL-OH.

290. The bifunctional compound according to claim 1 to any one of claims 61 to 129 or claims 276 to 282, wherein the bifunctional compound is:
(SEQ ID NO.: 158)
MGGTHHHHHHENLYFQGQVQLQESGGGLVQPGGSLRLSCAASGRTISR
YAMSWFRQAPGKEREFVAVARRSGDGAFYADSVQGRFTVSRDDAKNTV
YLQMNSLKPEDTAVYYCAIDSDTFYSGSYDYWGQGTQVTVSSEGGGGS
GGGGSGGGGSGGRQLL

291. The bifunctional compound according to claim 1 to any one of claims 61 to 129 or claims 276 to 282, wherein the T_{L} is the monoclonal antibody alirocumab (antibody with heavy chain of SEQ ID NO.: 66 and light chain of SEQ ID NO.: 67) conjugated to at least 1 unit -L_{I}-S_{L} consisting of:
(SEQ ID NO.: 175)
CGGSGGGGSGGGGSGGRQLL

292. The bifunctional compound according to claim 1 to any one of claims 61 to 129 or claims 276 to 282, wherein the T_{L} is the monoclonal antibody adalimumab (antibody with heavy chain of SEQ ID NO.: 68 and light chain of SEQ ID NO.: 69) conjugated to at least 1 unit -L_{I}-S_{L} selected from the group consisting of:
(SEQ ID NO: 175)
CGGSGGGGSGGGGSGGRQLL
(SEQ ID NO.: 184)
CREAPRWDAPLRDPALRQLL
(SEQ ID NO.: 186)
CREAPRWDAPLRDPALFQLL-OH

293. An isolated polynucleotide encoding the bifunctional compound according claim 290.

294. A vector comprising the polynucleotide according to claim 293.

295. The vector according to claim 294, wherein the vector is an expression vector, such as a bacterial vector or a viral vector.

296. A host cell comprising the polynucleotide according to claim 293 and/or the vector according to any one of claims 294 to 295.

297. The bifunctional compound according to any of the preceding claims wherein, said compound is able to form a ternary complex between sortilin and the target protein.

298. The bifunctional compound according to any of the preceding claims, wherein the compound is able to bind to bind to sortilin and the target protein at the same time.

299. The bifunctional compound according to any of the preceding claims wherein the dissociation constant of the binding of St to sortilin is of less than 50 μM, such as less than 2 μM, such as less than 0.5 μM, preferably less than 0.1 μM and the dissociation constant of the binding of T_{L} to its target is of less than 100 μM, such as less than 0.5 μM, such as less than 0.1 μM.

300. The bifunctional compound according to any of the preceding claims, wherein the compound is able to bind to sortilin at the cell surface.

301. The bifunctional compound according to any of the preceding claims wherein upon binding of S_{L} to sortilin located on the cell surface and binding of T_{L} to the target protein, the target protein is internalized into said cell.

302. The bifunctional compound according to claim 301, wherein the target protein is degraded after internalization into the cell.

303. The bifunctional compound according to any of claims 1 to 124, 138 to 151, 177 to 178, 183 to 192, 286 and 292 wherein, said compound is able to form a ternary complex between sortilin and TNFa.

304. The bifunctional compound according to any of claims 1 to 124, 138 to 151, 177 to 178, 183 to 191, 213 to 283, or 286 to 292 and 303, wherein the compound is able to bind to sortilin and TNFα at the same time.

305. The bifunctional compound according to any of claims 297 to 304, wherein the dissociation constant of the binding of St to sortilin is of less than 50 μM, such as less than 2 μM, such as less than 0.5 μM, preferably less than 0.1 μM and the dissociation constant of the binding of T_{L} to TNFa is of less than 100 μM, such as less than 0.5 μM, such as less than 0.1 μM.

306. The bifunctional compound according to any of claims 297 to 305, wherein the compound is able to bind to sortilin at the cell surface.

307. The bifunctional compound according to any of t claims 302 to 306, wherein upon binding of S_{L} to sortilin located on the cell surface and binding of T_{L} TNFa, TNFa is internalized into said cell.

308. The bifunctional compound according to any one of claims 307, wherein TNFa is degraded after internalization into the cell.

309. A pharmaceutical composition comprising a bifunctional compound according to any one of claims 1 to 308.

310. A bifunctional compound according to any one of claims 1 to 308 for use as a medicament.

311. A bifunctional compound according to any one of claims 1 to 308 for use in the treatment of a disorder or condition in a subject in need thereof.

312. The bifunctional compound for use according to claim 311, wherein the disorder or condition is mediated by an extracellular protein.

313. The bifunctional compound for use according to claim 312, wherein the extracellular protein is selected form the group consisting of: PCSK9, TNF-α, ANGPTL-3, an antibody light chain, IgG, IgE, IgA IL-1, IL-2, IL-6, IFN-γ, VEGF, TFG-β1, IL-21, IL-22, IL-5, IL-10, IL-8, cholinestearase, human CCL2, carboxypeptidase B-2, neutrophil elastase, Factor Xa, Factor XI, Factor XIa, Factor XII, Factor XIII, prothrombin, coagulation factor VII, coagulation factor IX, fibroblast growth factor 1, FGF-2, fibronectin 1, kallikrein-1, lipoprotein lipase, human matrix metallopeptidase 1, macrophage migration inhibitory factor, transformin growth factor-p (TGF-p), thrombospondin-1 (TSP-T), CD40 ligand, urokinase-type plasminogen activator, plasminogen activator tissue type (TPA), Plasminogen (PLG), Plasminogen Activator Inhibitor-1, Placenta Growth Factor, Phospholipase A2 Group IB, Phospholipase A2 Group IIA, Complement factor B, Complement factor D, complement factor H, Complement Component 5 and complement C1s.

314. The bifunctional compound for use according to claim 312, wherein the extracellular protein is PCSK9.

315. The bifunctional compound for use according to claim 314, wherein the disorder or condition is disorder of lipoprotein metabolism.

316. The bifunctional compound for use according to claim 315, wherein the disorder of lipoprotein disorder is linked to abnormal PCSK9 levels.

317. The bifunctional compound for use according to claim 315 or 316, wherein the disorder of lipoprotein disorder is selected from the group consisting of dyslipidemia, hypercholesterolemia and coronary heart disease.

318. The bifunctional compound for use according to claim 312, wherein the extracellular protein is TNF-α.

319. The bifunctional compound for use according to claim 318, wherein the disorder or condition is an inflammatory disease.

320. The bifunctional compound for use according to claim 318, wherein the disorder or condition is an autoimmune disease.

321. The bifunctional compound for use according to claim 318, wherein the disorder or condition is a cancer.

322. The bifunctional compound for use according to claim 311 or 318, wherein the disorder or condition is selected from the group consisting of rheumatoid arthritis, inflammatory bowel disease, graft-vs-host disease, ankylosing spondylitis, psoriasis, hidradenitis suppurativa, refractory asthma, systemic lupis erthyematosus, diabetes, and the induction of cachexia.

323. The bifunctional compound for use according to claim 312, wherein the extracellular protein is an antibody light chain or IgG.

324. The bifunctional compound for use according to claim 323, wherein the disorder or condition is selected from the group consisting of type 1 autoimmune pancreatitis, interstitial nephritis, Riedel's thyroiditis, storiform fibrosis, Mikulicz's disease, Kuttner's tumor, inflammatory' pseudotumors, mediastinal fibrosis, retroperitoneal fibrosis (Ormond's disease), aortitis, periaortitis, proximal biliary strictures, idiopathic hypocomplementic tubulointerstitial nephritis, multifocal fibrosclerosis, pachymeningitis, pancreatic enlargement, tumefactive lesions, pericarditis, rheumatoid arthritis (RA), inflammatory bowel disease, multiple sclerosis, myasthenic gravis, thyroid eye disease, chronic inflammatory demyelinating polyneuropathy, warm autoimmune hemolytic anemia, ankylosing spondylitis, primary Sjogren's syndrome, psoriatic arthritis, and systemic lupus erythematosus (SLE), sclerosing cholangitis, and IgG monoclonal gammopathy, monoclonal gammopathy of undetermined significance (MGUS).

325. The bifunctional compound for use according to any one of claims 310 to 324, wherein the extracellular protein is present in abnormally high levels.

326. The bifunctional compound for use according to any one of claims 310 to 324, wherein the extracellular protein is mutated or misfolded compared to the corresponding endogenous protein.

327. The bifunctional compound for use according to any one of claims 310 to 326, wherein the subject is a mammal.

328. The compound for the use according to claim 327, wherein the mammal is a human.

329. A method of targeted lysosomal degradation of an extracellular protein, comprising administering an effective amount of the bifunctional compound according to any one of claims 1 to 308.

330. A method of removal of an extracellular target protein from the plasma of a patient or subject in need thereof, comprising administering a bifunctional compound according to any one of claims 1 to 308.

331. Use of a bifunctional compound according to any one of claims 1 to 308 for the manufacture of a medicament for the treatment of a disease or condition.

332. A method of treatment of a disease or condition comprising administering a bifunctional compound according to any one of claims 1 to 312 to a subject in need thereof.

333. Use of a bifunctional compound according to any one of claims 1 to 312 for the manufacture of a medicament for the treatment of a disorder or condition mediated by an extracellular protein.

334. A method of treatment of a disorder or condition mediated by an extracellular protein, comprising administering a bifunctional compound according to any one of claims 1 to 312 to a subject in need thereof.

335. A method of targeted lysosomal degradation of TNFa, comprising administering an effective amount of the bifunctional compound according to any one of claims 1 to 124, 138 to 151, 177 to 178, 183 to 192, 286 to 292 and 303 to 308 a subject in need thereof.

336. A method of removal of TNFa from the plasma of a subject in need thereof, comprising administering a bifunctional compound according to any one of claims 1 to 124, 138 to 151, 177 to 178, 183 to 192, 286 to 292 and 303 to 308.

337. The method according to any one of claim 335 or 336, wherein the compound is selected form any one of compounds BF030 to BF149 in Table A of the section “List of compounds”, or a pharmaceutically acceptable salt thereof.

338. Use of a bifunctional compound according to any one of claims 1 to 308 for the manufacture of a medicament for the treatment of a disease or condition.

339. A method of treatment of a disease or condition comprising administering a bifunctional compound according to any one of claims 1 to 308 to a subject in need thereof.

340. Use of a bifunctional compound according to any one of claims 1 to 124, 138 to 151, 177 to 178, 183 to 192, 286 to 292 and 303 to 308 for the manufacture of a medicament for the treatment of a disorder or condition mediated by TNFa.

341. A method of treatment of a disorder or condition mediated by TNFa, comprising administering a bifunctional compound according to any one of claims 1 to 124, 138 to 151, 177 to 178, 183 to 192, 286 to 292 and 303 to 308 to a subject in need thereof.

342. A compound having a structure according to formula (III): 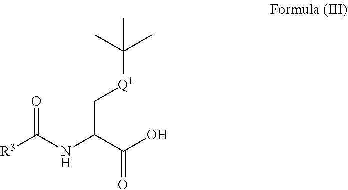
or a pharmaceutically acceptable salt thereof,
wherein Q¹ is a bond or —CH₂—; and
R³ is of Formula (IIIp) or Formula (IIIq) or formula B-II: 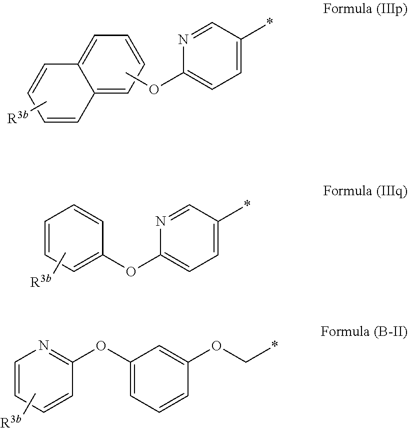
wherein R^{3b} is selected from H, halogen, alkoxy, —CF₃, and an optionally substituted C₁₋C₅ alkyl, wherein one or more methylene group(s) of the C₁₋₅ alkyl are optionally individually replaced by one or more of the groups consisting of —O—, —NH—, —C(O)—, ester, amide, carbamate, thiourea, sulphonamide, urea, 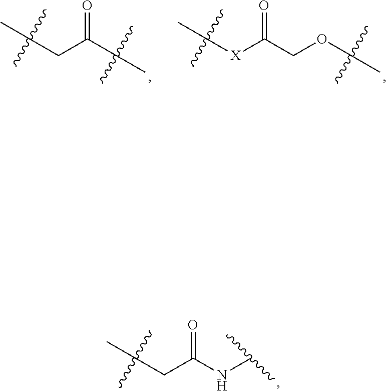
an optionally substituted carbocycle; an optionally substituted heterocycle and 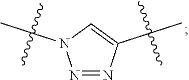 [SMILES: *c1cn(*)nn1]
wherein X an atom selected from N and O;
with the proviso that R^{3b} is not H when R³ is of formula (IIIq); and
wherein * denotes the attachment with Formula (III).

343. The compound according to claim 342, wherein Q¹ is —CH₂—.

344. The compound according to claim 342, wherein Q¹ is a bond.

345. The compound according to any one of claims 342 to 344, wherein R³ is of Formula (IIIr): 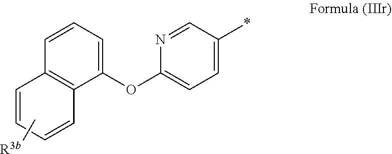
wherein R^{3b} is selected from H, halogen, alkoxy, —CF₃, and an optionally substituted C₁₋C₅ alkyl, wherein one or more methylene group(s) of the C₁₋₅ alkyl are optionally individually replaced by one or more of the groups consisting of —O—, —NH—, —C(O)—, ester, amide, carbamate, thiourea and 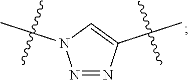 [SMILES: *c1cn(*)nn1]
and
wherein * denotes the attachment with Formula (III).

346. The compound according to claims 342 to 345, wherein the compound is of Formula (IIIs), Formula (IIIt) or formula B-III: 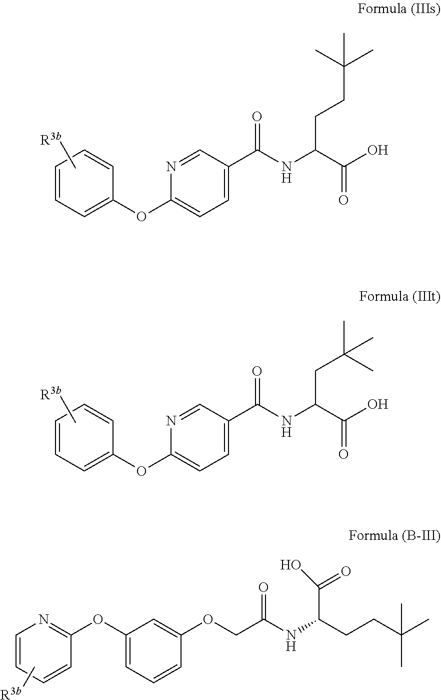
wherein R^{3b} is selected from H, halogen, alkoxy, —CF₃, and an optionally substituted C₁-C₅ alkyl, wherein one or more methylene group(s) of the C₁₋₅ alkyl are optionally individually replaced by one or more of the groups consisting of —O—, —NH—, —C(O)—, ester, amide, carbamate, thiourea sulphonamide, urea, 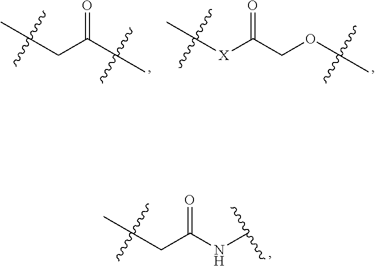
an optionally substituted carbocycle; an optionally substituted heterocycle and 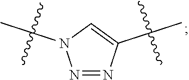
wherein X is N (H) or O; with the proviso that R^{3b} is not H when R³ is of formula (IIIt).

347. The compound according to any one of claims 342 to 346, wherein R^{3b} is H.

348. The compound according to any one of claims 342 to 346, wherein R^{3b} is halogen or —CF₃.

349. The compound according to any one of claims 342 to 346, wherein R^{3b} is C₁₋C₅ alkyl, wherein one or more methylene group(s) of the C₁₋₅ alkyl are optionally individually replaced by one or more of the groups consisting of —O—, —NH—, —C(O)—, ester, amide, carbamate, thiourea and

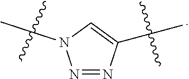 [SMILES: *c1cn(*)nn1]

350. The compound according to any one of claim 342 to 346 or 349, wherein R^{3b} is C₁₋C₅ alkyl, wherein one or more methylene group(s) of the C₁₋₅ alkyl are optionally individually replaced by one or more of the groups consisting of —O—, amide, and

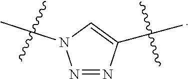 [SMILES: *c1cn(*)nn1]

351. The compound according to any one of claims 342 to 346 or 349 to 350, wherein R^{3b} is C₁₋C₅ alkyl, wherein one or more methylene group(s) of the C₁₋₅ alkyl are optionally individually replaced by —O—.

352. The compound according to any one of claims 342 to 346 or 349 to 351, wherein R^{3b} is C₁-C₅ alkyl, wherein one or more methylene group of the C₁₋₅ alkyl is optionally individually replaced by an amide.

353. The compound according to any one of claims 342 to 346 or 349 to 352, wherein R^{3b} is C₁₋C₅ alkyl, wherein one or more methylene group of the C₁₋₅ alkyl is optionally individually replaced by

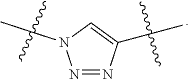 [SMILES: *c1cn(*)nn1]

354. The compound according to any one of claims 342 to 346, wherein R^{3b} is —C(═O)—(NH)—CH₃.

355. The compound according to any one of claims 342 to 346, wherein R^{3b} is —CH₂—CH₂—O—CH₃.

356. The compound according to any one of claims 342 to 346, wherein R^{3b} is —O—CH₂—CH₂—O—CH₃.

357. The compound according to any one of claims 342 to 346, wherein R^{3b} is —CH₂—NH—C(═O)—CH₃.

358. The compound according to any one of claims 342 to 346, wherein R^{3b} is, —O—CH₂—CH₂—NH—C(═O)—CH₃.

359. The compound according to any one of claims 342 to 346, wherein R^{3b} is tetrazole-CH₂—CH₂—O—CH₃.

360. The compound according to any one of claims 342 to 346, wherein R^{3b} is C₁₋C₅ alkyl, wherein one or more methylene group of the C₁₋₅ alkyl is optionally individually replaced by an optionally substituted carbocycle, such as a carbocycle according to 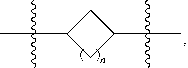
wherein n is an integer selected from 0, 1, 2 or 3.

361. The compound according to any one of claims 59 to 346, wherein R^{3b} is C₁₋C₅ alkyl, wherein one or more methylene group of the C₁₋₅ alkyl is optionally individually replaced by an optionally substituted heterocycle, such as an heterocycle according to 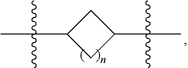
wherein n is an integer selected from 0, 1, 2 or 3; or an heterocycle according to

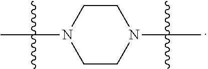 [SMILES: *N1CCN(*)CC1]

362. The compound according to any one of claims 342 to 346, wherein R^{3b} is —O—CH₂—C(═O)N(H)R^{IIIa}, wherein R^{IIIa} is selected from H, and C₁-C₆ alkyl.

363. The compound according t to any one of claims 342 to 346, wherein R^{3b} is —O—CH₂—CO₂R^{IIIa}, wherein R^{IIIa} is selected from H, and C₁-C₆ alkyl.

364. The compound according to any one of claims 342 to 346, wherein R^{3b} is C₁₋C₅ alkyl, wherein one or more methylene group of the C₁₋₅ alkyl is optionally individually replaced by one of the groups shown in Table Z in the section “Linkers”.

365. The compound according to any one of the preceding claims, wherein the compound is the S-stereoisomer.

366. The compound according to any one of the preceding claims, wherein the compound is the R-stereoisomer.

367. The compound according to any one of claims 342 to 347, wherein the compound is:

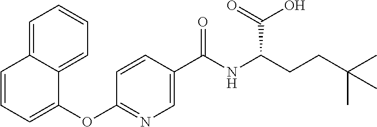 [SMILES: CC(C)(C)CC[C@H](NC(=O)c1ccc(Oc2cccc3ccccc23)nc1)C(=O)O]

368. The compound according to any one of claims 342 to 346, wherein the compound is:

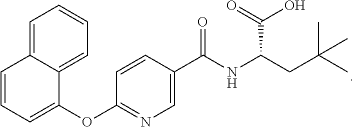 [SMILES: CC(C)(C)C[C@H](NC(=O)c1ccc(Oc2cccc3ccccc23)nc1)C(=O)O]

369. The compound according to any one of claims 342 to 346 or 349 to 352, wherein the compound is selected from any one of compounds SB001, SB003, SB006, SB007, SB008, SB009, SB010, SB011 and SB012 according to Table B in the section “List of Compounds” or a pharmaceutically acceptable salt thereof.

370. A compound selected from the group consisting of 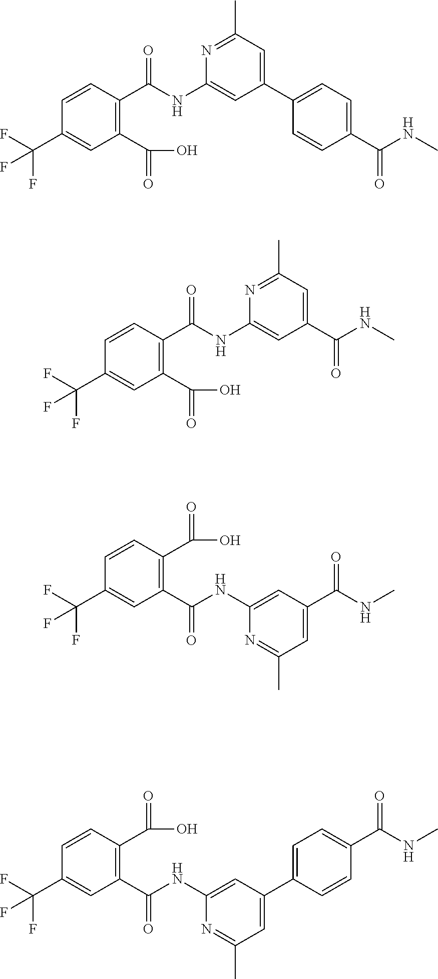 [SMILES: CNC(=O)c1ccc(-c2cc(C)nc(NC(=O)c3ccc(C(F)(F)F)cc3C(=O)O)c2)cc1 ; CNC(=O)c1cc(C)nc(NC(=O)c2ccc(C(F)(F)F)cc2C(=O)O)c1 ; CNC(=O)c1cc(C)nc(NC(=O)c2cc(C(F)(F)F)ccc2C(=O)O)c1 ; CNC(=O)c1ccc(-c2cc(C)nc(NC(=O)c3cc(C(F)(F)F)ccc3C(=O)O)c2)cc1]
or a pharmaceutically acceptable salt thereof.

371. A compound having a structure selected from any one of: 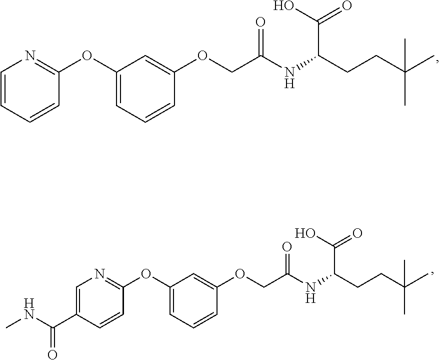
or a pharmaceutically acceptable salt thereof.

372. The compound according to any one of claims 342 to 372, wherein the compound has is able to bind sortilin with a dissociation constant of at least constant of less than 50 μM, such as less than 40 μM, such as less than 30 μM, such as less than 20 μM, such as less than 10 μM, such as less than 5 μM, such as less than 4 μM, such as less than 3 μM, such as less than 2 μM, such as less than 1 μM, such as less than 0.8 μM, such as less than 0.6 μM, such as less than 0.5 μM, such as less than 0.4 μM, such as less than 0.3 μM, such as less than 0.2 μM, such as less than 0.1 μM.


SEQUENCE LISTING (197 sequences)

SEQ ID NO: 1 - protein, 831 aa, Homo sapiens [sequence omitted]

SEQ ID NO: 2 - protein, 762 aa, Homo sapiens
  REGION 1..762: note = MISC_FEATURE - sortilin-6his
MERPWGAADGLSRWPHGLGLLLLLQLLPPSTLSQDRLDAPPPPAAPLPRWSGPIGVSWGLRAAAAGGAFPRGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSMCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSHHHHHH

SEQ ID NO: 3 - protein, 593 aa, Homo sapiens
  REGION 1..593: note = MISC_FEATURE - progranulin
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 4 - protein, 13 aa, synthetic construct
  REGION 1..13: note = synthetic
  REGION 1..13: note = MISC_FEATURE - neurotensin
QLYENKPRRPYIL

SEQ ID NO: 5 - protein, 4 aa, synthetic construct
  REGION 1..4: note = synthetic
  REGION 1..4: note = MISC_FEATURE - neurotensin 4aa C-terminal
PYIL

SEQ ID NO: 6 - protein, 6 aa, synthetic construct
  REGION 1..6: note = synthetic
  REGION 1..6: note = MISC_FEATURE - neurotensin 6 aa N-term
QLYENK

SEQ ID NO: 7 - protein, 22 aa, synthetic construct
  REGION 1..22: note = synthesized
  PEPTIDE 1..22
IKLSGGVQAKAGVINMDKSESM

SEQ ID NO: 8 - protein, 22 aa, synthetic construct
  REGION 1..22: note = synthetic
  PEPTIDE 1..22
IKLSGGVQAKAGVINMFKSESY

SEQ ID NO: 9 - protein, 23 aa, synthetic construct
  REGION 1..23: note = synthetic
  PEPTIDE 1..22
IKLSGGVQAKAGVINMFKSESYK

SEQ ID NO: 10 - protein, 17 aa, synthetic construct
  REGION 1..17: note = synthetic
  PEPTIDE 1..17
GVQAKAGVINMFKSESY

SEQ ID NO: 11 - protein, 17 aa, synthetic construct
  REGION 1..17: note = synthetic
  PEPTIDE 1..17
GVRAKAGVRNMFKSESY

SEQ ID NO: 12 - protein, 17 aa, synthetic construct
  REGION 1..17: note = synthetic
  SITE 11: note = MISC_FEATURE - Norleucine
GVRAKAGVRNXFKSESY

SEQ ID NO: 13 - protein, 24 aa, synthetic construct
  REGION 1..24: note = synthetic
  PEPTIDE 1..24
YKSLRRKAPRWDAPLRDPALRQLL

SEQ ID NO: 14 - protein, 24 aa, synthetic construct
  REGION 1..24: note = synthetic
YKSLRRKAPRWDAYLRDPALRQLL

SEQ ID NO: 15 - protein, 24 aa, synthetic construct
  REGION 1..24: note = synthetic
  PEPTIDE 1..24
YKSLRRKAPRWDAYLRDPALRPLL

SEQ ID NO: 16 - skipped

SEQ ID NO: 17 - protein, 15 aa, synthetic construct
  REGION 1..15: note = synthetic
  PEPTIDE 1..15
REAPRWDAPLRDPAL

SEQ ID NO: 18 - protein, 15 aa, synthetic construct
  REGION 1..15: note = synthetic
  PEPTIDE 1..5
REALRWDAPLRDPAP

SEQ ID NO: 19 - protein, 15 aa, synthetic construct
  REGION 1..15: note = synthetic
  PEPTIDE 1..15
PYILKRQLYENKPRR

SEQ ID NO: 20 - protein, 7 aa, synthetic construct
  REGION 1..7: note = synthetic
LYENKPR

SEQ ID NO: 21 - protein, 8 aa, synthetic construct
  REGION 1..8: note = synthetic
  PEPTIDE 1..8
APLRDPAP

SEQ ID NO: 22 - protein, 4 aa, synthetic construct
  REGION 1..4: note = synthetic
  PEPTIDE 1..4
RQLL

SEQ ID NO: 23 - protein, 4 aa, synthetic construct
  REGION 1..4: note = synthetic
  PEPTIDE 1..4
FQLL

SEQ ID NO: 24 - protein, 4 aa, synthetic construct
  REGION 1..4: note = synthetic
  PEPTIDE 1..4
KQLL

SEQ ID NO: 25 - protein, 4 aa, synthetic construct
  REGION 1..4: note = synthetic
  PEPTIDE 1..4
PQLL

SEQ ID NO: 26 - protein, 4 aa, synthetic construct
  REGION 1..4: note = synthetic
  PEPTIDE 1..4
PYIL

SEQ ID NO: 27 - protein, 4 aa, synthetic construct
  REGION 1..4: note = synthetic
  PEPTIDE 1..4
RYLL

SEQ ID NO: 28 - protein, 4 aa, synthetic construct
  REGION 1..4: note = synthetic
  PEPTIDE 1..4
FYLL

SEQ ID NO: 29 - protein, 4 aa, synthetic construct
  REGION 1..4: note = synthetic
  PEPTIDE 1..4
YQLL

SEQ ID NO: 30 - protein, 4 aa, synthetic construct
  REGION 1..4: note = synthetic
  PEPTIDE 1..4
LLQL

SEQ ID NO: 31 - protein, 4 aa, synthetic construct
  REGION 1..4: note = synthetic
FYIL

SEQ ID NO: 32 - protein, 4 aa, synthetic construct
  REGION 1..4: note = synthetic
  PEPTIDE 1..4
RYIL

SEQ ID NO: 33 - protein, 4 aa, synthetic construct
  REGION 1..4: note = synthetic
  PEPTIDE 1..4
PYLL

SEQ ID NO: 34 - protein, 4 aa, synthetic construct
  REGION 1..4: note = synthetic
  PEPTIDE 1..4
PYYL

SEQ ID NO: 35 - protein, 4 aa, synthetic construct
  REGION 1..4: note = synthetic
  PEPTIDE 1..4
PYFL

SEQ ID NO: 36 - protein, 4 aa, synthetic construct
  REGION 1..4: note = synthetic
  PEPTIDE 1..4
RYYL

SEQ ID NO: 37 - protein, 4 aa, synthetic construct
  REGION 1..4: note = synthetic
  PEPTIDE 1..4
RYFL

SEQ ID NO: 38 - protein, 4 aa, synthetic construct
  REGION 1..4: note = synthetic
  PEPTIDE 1..4
RQFL

SEQ ID NO: 39 - protein, 4 aa, synthetic construct
  REGION 1..4: note = synthetic
  PEPTIDE 1..4
RQYL

SEQ ID NO: 40 - protein, 4 aa, synthetic construct
  REGION 1..4: note = synthetic
  PEPTIDE 1..4
LQLL

SEQ ID NO: 41 - protein, 4 aa, synthetic construct
  REGION 1..4: note = synthetic
  PEPTIDE 1..4
HQLL

SEQ ID NO: 42 - protein, 4 aa, synthetic construct
  REGION 1..4: note = synthetic
  PEPTIDE 1..4
IQLL

SEQ ID NO: 43 - protein, 4 aa, synthetic construct
  REGION 1..4: note = synthetic
  PEPTIDE 1..4
FYYL

SEQ ID NO: 44 - protein, 14 aa, synthetic construct
  REGION 1..14: note = synthetic
PYMKLAPGELTIIL

SEQ ID NO: 45 - protein, 12 aa, synthetic construct
  REGION 1..12: note = synthetic
  PEPTIDE 1..4
NEKLSQLQTYMI

SEQ ID NO: 46 - protein, 17 aa, synthetic construct
  REGION 1..17: note = synthetic
  PEPTIDE 1..4
KDADLYTSRVMLSSQVP

SEQ ID NO: 47 - protein, 15 aa, synthetic construct
  REGION 1..15: note = synthetic
  PEPTIDE 1..4
  SITE 4: note = MISC_FEATURE - amidated C-term
RLFKKRRLRSPRVLF

SEQ ID NO: 48 - protein, 20 aa, synthetic construct
  REGION 1..20: note = synthetic
  SITE 4: note = MISC_FEATURE - amidated C-term
ITVDPRLFKKRRLRSPRVLF

SEQ ID NO: 49 - protein, 26 aa, synthetic construct
  REGION 1..26: note = synthetic
  PEPTIDE 1..26
ITVDPRLFKKRRLRSPRVLFSTQPPR

SEQ ID NO: 50 - protein, 21 aa, synthetic construct
  REGION 1..21: note = synthetic
  PEPTIDE 1..21
WSGPIGVSWGLRAAAAGGAFP

SEQ ID NO: 51 - protein, 28 aa, synthetic construct
  REGION 1..28: note = synthetic
  PEPTIDE 1..28
WSGPIGVSWGLRAAAAGGAFPRGGRWRR

SEQ ID NO: 52 - protein, 7 aa, synthetic construct
  REGION 1..7: note = synthetic
  PEPTIDE 1..7
GVSWGLR

SEQ ID NO: 53 - protein, 19 aa, synthetic construct
  REGION 1..19: note = synthetic
  PEPTIDE 1..19
REAPRWDAPLRDPALRQLL

SEQ ID NO: 54 - protein, 19 aa, synthetic construct
  REGION 1..19: note = synthetic
  PEPTIDE 1..19
REALRWDAPLRDPAPRQLL

SEQ ID NO: 55 - protein, 19 aa, synthetic construct
  REGION 1..19: note = synthetic
  PEPTIDE 1..19
PYILKRQLYENKPRRPYIL

SEQ ID NO: 56 - protein, 12 aa, synthetic construct
  REGION 1..12: note = synthetic
  PEPTIDE 1..12
LYENKPRRPYIL

SEQ ID NO: 57 - protein, 12 aa, synthetic construct
  REGION 1..12: note = synthetic
  PEPTIDE 1..12
APLRDPAPRQLL

SEQ ID NO: 58 - protein, 19 aa, synthetic construct
  REGION 1..19: note = synthetic
  PEPTIDE 1..19
REAPRWDAPLRDPALFQLL

SEQ ID NO: 59 - protein, 19 aa, synthetic construct
  REGION 1..19: note = synthetic
  PEPTIDE 1..19
REAPRWDAPLRDPALRYLL

SEQ ID NO: 60 - protein, 19 aa, synthetic construct
  REGION 1..19: note = synthetic
  PEPTIDE 1..19
REAPRWDAPLRDPALRQYL

SEQ ID NO: 61 - protein, 19 aa, synthetic construct
  REGION 1..19: note = synthetic
  PEPTIDE 1..19
REAPRWDAPLRDPALRYYL

SEQ ID NO: 62 - protein, 5 aa, synthetic construct
  REGION 1..5: note = synthetic
  PEPTIDE 1..5
TGGFM

SEQ ID NO: 63 - protein, 5 aa, synthetic construct
  REGION 1..5: note = synthetic
  PEPTIDE 1..5
YGGFL

SEQ ID NO: 64 - protein, 4 aa, synthetic construct
  REGION 1..4: note = synthetic
  SITE 4: note = MISC_FEATURE - amidated C-term
LYEN

SEQ ID NO: 65 - protein, 5 aa, synthetic construct
  REGION 1..5: note = synthetic
  REGION 1..5: note = MISC_FEATURE - amidated C-term
LYENK

SEQ ID NO: 66 - protein, 447 aa, synthetic construct
  REGION 1..447: note = heavy chain of antibody alirocumab
  REGION 1..447: note = MISC_FEATURE - heavy chain of antibody
EVQLVESGGGLVQPGGSLRLSCAASGFTFNNYAMNWVRQAPGKGLDWVSTISGSGGTTNYADSVKGRFIISRDSSKHTLYLQMNSLRAEDTAVYYCAKDSNWGNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 220 aa, synthetic construct
  REGION 1..220: note = light chain of antibody alirocumab
  REGION 1..220: note = MISC_FEATURE - light chain of antibody
DIVMTQSPDSLAVSLGERATINCKSSQSVLYRSNNRNFLGWYQQKPGQPPNLLIYWASTRESGVPDRFSGSGSGTDFTLTISSLQAEDVAVYYCQQYYTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 451 aa, synthetic construct
  REGION 1..451: note = heavy chain of adalimumab
  REGION 1..451: note = MISC_FEATURE - heavy chain of antibody
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 214 aa, synthetic construct
  REGION 1..214: note = light chain of antibody adalimumab
  REGION 1..214: note = MISC_FEATURE - light chain of antibody
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 139 aa, synthetic construct
  REGION 1..139: note = synthetic
  REGION 1..139: note = MISC_FEATURE - nanobody
MGGTHHHHHHENLYFQGQVQLQESGGGLVQPGGSLRLSCAASGRTISRYAMSWFRQAPGKEREFVAVARRSGDGAFYADSVQGRFTVSRDDAKNTVYLQMNSLKPEDTAVYYCAIDSDTFYSGSYDYWGQGTQVTVSSE

SEQ ID NO: 71 - protein, 156 aa, synthetic construct
  REGION 1..156: note = synthetic
  REGION 1..156: note = MISC_FEATURE - nanobody with linker
MGGTHHHHHHENLYFQGQVQLQESGGGLVQPGGSLRLSCAASGRTISRYAMSWFRQAPGKEREFVAVARRSGDGAFYADSVQGRFTVSRDDAKNTVYLQMNSLKPEDTAVYYCAIDSDTFYSGSYDYWGQGTQVTVSSEGGGGSGGGGSGGGGSGG

SEQ ID NO: 72 - protein, 6 aa, synthetic construct
  REGION 1..6: note = synthetic
  SITE 1: note = MISC_FEATURE - X represent the moiety targeting extracellular molecule
  SITE 2: note = MISC_FEATURE - X represent the linker (LI) or a bond
XXRQLL

SEQ ID NO: 73 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXFQLL

SEQ ID NO: 74 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXKQLL

SEQ ID NO: 75 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXPQLL

SEQ ID NO: 76 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXPYIL

SEQ ID NO: 77 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXRYLL

SEQ ID NO: 78 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXFYLL

SEQ ID NO: 79 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXYQLL

SEQ ID NO: 80 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXLLQL

SEQ ID NO: 81 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXFYIL

SEQ ID NO: 82 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXRYIL

SEQ ID NO: 83 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXPYLL

SEQ ID NO: 84 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXPYYL

SEQ ID NO: 85 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXPYFL

SEQ ID NO: 86 - protein, 6 aa, synthetic construct
  REGION 1..6: note = synthetic
  SITE 1: note = MISC_FEATURE - X represents the moiety targeting extracellular molecule
  SITE 2: note = misc_feature - Xaa can be any naturally occurring amino acid
XXRYYL

SEQ ID NO: 87 - protein, 6 aa, synthetic construct
  REGION 1..6: note = synthetic
  SITE 1: note = MISC_FEATURE - X represent the moiety targeting the extracellular molecule
  SITE 2: note = MISC_FEATURE - X represent the linker (LI) or a bond
XXRYFL

SEQ ID NO: 88 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXRQFL

SEQ ID NO: 89 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXRQYL

SEQ ID NO: 90 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXLQLL

SEQ ID NO: 91 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXHQLL

SEQ ID NO: 92 - protein, 6 aa, synthetic construct
  REGION 1..6: note = synthetic
  SITE 1: note = MISC_FEATURE - X represents the moiety targeting extracellular molecule
  SITE 2: note = misc_feature - Xaa can be any naturally occurring amino acid
XXIQLL

SEQ ID NO: 93 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
XXFYYL

SEQ ID NO: 94 - protein, 16 aa, synthetic construct
  REGION 1..16: note = synthetic
  SITE 1: note = MISC_FEATURE - X represents the moiety targeting extracellular molecule
  SITE 2: note = MISC_FEATURE - X represent the linker (LI) or a bond
XXPYMKLAPGELTIIL

SEQ ID NO: 95 - protein, 14 aa, synthetic construct
  REGION 1..14: note = synthetic
  SITE 1: note = MISC_FEATURE - X represents the moiety targeting extracellular molecule
  SITE 2: note = MISC_FEATURE - X represent the linker (LI) or a bond
XXNEKLSQLQTYMI

SEQ ID NO: 96 - protein, 19 aa, synthetic construct
  REGION 1..19: note = synthetic
  SITE 1: note = MISC_FEATURE - X represents the moiety targeting extracellular molecule
  SITE 2: note = MISC_FEATURE - X represent the linker (LI) or a bond
XXKDADLYTSRVMLSSQVP

SEQ ID NO: 97 - protein, 17 aa, synthetic construct
  REGION 1..17: note = synthetic
  SITE 1: note = misc_feature - X represents the moiety targeting extracellular molecule
  SITE 2: note = misc_feature - X represent the linker (LI) or a bond
  SITE 17: note = misc_feature - amidated C-term
XXRLFKKRRLRSPRVLF

SEQ ID NO: 98 - protein, 22 aa, synthetic construct
  REGION 1..22: note = synthetic
  SITE 1: note = MISC_FEATURE - X represents the moiety targeting extracellular molecule
  SITE 2: note = MISC_FEATURE - X represent the linker (LI) or a bond
  SITE 17: note = MISC_FEATURE - amidated C-term
XXITVDPRLFKKRRLRSPRVLF

SEQ ID NO: 99 - protein, 28 aa, synthetic construct
  REGION 1..28: note = synthetic
  SITE 1: note = MISC_FEATURE - X represents the moiety targeting extracellular molecule
  SITE 2: note = MISC_FEATURE - X represent the linker (LI) or a bond
XXITVDPRLFKKRRLRSPRVLFSTQPPR

SEQ ID NO: 100 - protein, 23 aa, synthetic construct
  REGION 1..23: note = synthetic
  SITE 1: note = MISC_FEATURE - X represents the moiety targeting extracellular molecule
  SITE 2: note = MISC_FEATURE - X represent the linker (LI) or a bond
XXWSGPIGVSWGLRAAAAGGAFP

SEQ ID NO: 101 - protein, 30 aa, synthetic construct
  REGION 1..30: note = synthetic
  SITE 1: note = MISC_FEATURE - X represents the moiety targeting extracellular molecule
  SITE 2: note = MISC_FEATURE - X represent the linker (LI) or a bond
XXWSGPIGVSWGLRAAAAGGAFPRGGRWRR

SEQ ID NO: 102 - protein, 9 aa, synthetic construct
  REGION 1..9: note = synthetic
  SITE 1: note = MISC_FEATURE - X represents the moiety targeting extracellular molecule
  SITE 2: note = MISC_FEATURE - X represent the linker (LI) or a bond
XXGVSWGLR

SEQ ID NO: 103 - protein, 21 aa, synthetic construct
  REGION 1..21: note = synthetic
  SITE 1: note = MISC_FEATURE - X represents the moiety targeting extracellular molecule
  SITE 2: note = MISC_FEATURE - X represent the linker (LI) or a bond
XXREAPRWDAPLRDPALRQLL

SEQ ID NO: 104 - protein, 21 aa, synthetic construct
  REGION 1..21: note = synthetic
  SITE 1: note = MISC_FEATURE - X represents the moiety targeting extracellular molecule
  SITE 2: note = MISC_FEATURE - X represent the linker (LI) or a bond
XXREALRWDAPLRDPAPRQLL

SEQ ID NO: 105 - protein, 21 aa, synthetic construct
  REGION 1..21: note = synthetic
  SITE 1: note = MISC_FEATURE - X represents the moiety targeting extracellular molecule
  SITE 2: note = MISC_FEATURE - X represent the linker (LI) or a bond
XXPYILKRQLYENKPRRPYIL

SEQ ID NO: 106 - protein, 14 aa, synthetic construct
  REGION 1..14: note = synthetic
  SITE 1: note = MISC_FEATURE - X represents the moiety targeting extracellular molecule
  SITE 2: note = MISC_FEATURE - X represent the linker (LI) or a bond
XXLYENKPRRPYIL

SEQ ID NO: 107 - protein, 14 aa, synthetic construct
  REGION 1..14: note = synthetic
  SITE 1: note = MISC_FEATURE - X represents the moiety targeting extracellular molecule
  SITE 2: note = MISC_FEATURE - X represent the linker (LI) or a bond
XXAPLRDPAPRQLL

SEQ ID NO: 108 - protein, 21 aa, synthetic construct
  REGION 1..21: note = synthetic
  SITE 1: note = MISC_FEATURE - X represents the moiety targeting extracellular molecule
  SITE 2: note = MISC_FEATURE - X represent the linker (LI) or a bond
XXREAPRWDAPLRDPALFQLL

SEQ ID NO: 109 - protein, 21 aa, synthetic construct
  REGION 1..21: note = synthetic
  SITE 1: note = MISC_FEATURE - X represents the moiety targeting extracellular molecule
  SITE 2: note = MISC_FEATURE - X represent the linker (LI) or a bond
XXREAPRWDAPLRDPALRYLL

SEQ ID NO: 110 - protein, 21 aa, synthetic construct
  REGION 1..21: note = synthetic
  SITE 1: note = MISC_FEATURE - X represents the moiety targeting extracellular molecule
  SITE 2: note = MISC_FEATURE - X represent the linker (LI) or a bond
XXREAPRWDAPLRDPALRQYL

SEQ ID NO: 111 - protein, 21 aa, synthetic construct
  REGION 1..21: note = synthetic
  SITE 1: note = MISC_FEATURE - X represents the moiety targeting extracellular molecule
  SITE 2: note = MISC_FEATURE - X represent the linker (LI) or a bond
XXREAPRWDAPLRDPALRYYL

SEQ ID NO: 112 - protein, 7 aa, synthetic construct
  REGION 1..7: note = synthetic
  SITE 1: note = MISC_FEATURE - X represents the moiety targeting extracellular molecule
  SITE 2: note = MISC_FEATURE - X represent the linker (LI) or a bond
XXTGGFM

SEQ ID NO: 113 - protein, 7 aa, synthetic construct
  REGION 1..7: note = synthetic
  SITE 1: note = MISC_FEATURE - X represents the moiety targeting extracellular molecule
  SITE 2: note = MISC_FEATURE - X represent the linker (LI) or a bond
XXYGGFL

SEQ ID NO: 114 - protein, 6 aa, synthetic construct
  REGION 1..6: note = synthetic
  SITE 1: note = MISC_FEATURE - X represents the moiety targeting extracellular molecule
  SITE 2: note = MISC_FEATURE - X represent the linker (LI) or a bond
  SITE 6: note = MISC_FEATURE - amidated C-term
XXLYEN

SEQ ID NO: 115 - protein, 7 aa, synthetic construct
  REGION 1..7: note = synthetic
  SITE 1: note = MISC_FEATURE - X represents the moiety targeting extracellular molecule
  SITE 2: note = MISC_FEATURE - X represent the linker (LI) or a bond
  SITE 7: note = MISC_FEATURE - amidated C-term
XXLYENK

SEQ ID NO: 116 - skipped

SEQ ID NO: 117 - protein, 5 aa, synthetic construct
  REGION 1..5: note = synthetic
  SITE 1: note = MISC_FEATURE - conjugated to biotin
  SITE 1: note = MISC_FEATURE - X represent the linker (LI) or a bond
XRQLL

SEQ ID NO: 118 - protein, 5 aa, synthetic construct
  REGION 1..5: note = synthetic
  SITE 1: note = MISC_FEATURE - conjugated to biotin
  SITE 1: note = MISC_FEATURE - X represent the linker (LI) or a bond
XFQLL

SEQ ID NO: 119 - protein, 5 aa, synthetic construct
  REGION 1..5: note = synthetic
  SITE 1: note = MISC_FEATURE - conjugated to biotin
  SITE 1: note = MISC_FEATURE - X represent the linker (LI) or a bond
XRYLL

SEQ ID NO: 120 - protein, 5 aa, synthetic construct
  REGION 1..5: note = synthetic
  SITE 1: note = MISC_FEATURE - conjugated to biotin
  SITE 1: note = MISC_FEATURE - X represent the linker (LI) or a bond
XFYLL

SEQ ID NO: 121 - protein, 5 aa, synthetic construct
  REGION 1..5: note = synthetic
  SITE 1: note = MISC_FEATURE - conjugated to biotin
  SITE 1: note = MISC_FEATURE - X represent the linker (LI) or a bond
XYQLL

SEQ ID NO: 122 - protein, 20 aa, synthetic construct
  REGION 1..20: note = synthetic
  SITE 1: note = MISC_FEATURE - conjugated to biotin
  SITE 1: note = MISC_FEATURE - X represent the linker (LI) or a bond
XREAPRWDAPLRDPALRQLL

SEQ ID NO: 123 - protein, 20 aa, synthetic construct
  REGION 1..20: note = synthetic
  SITE 1: note = MISC_FEATURE - conjugated to biotin
  SITE 1: note = MISC_FEATURE - X represent the linker (LI) or a bond
XREALRWDAPLRDPAPRQLL

SEQ ID NO: 124 - protein, 20 aa, synthetic construct
  REGION 1..20: note = synthetic
  SITE 1: note = MISC_FEATURE - conjugated to biotin
  SITE 1: note = MISC_FEATURE - X represent the linker (LI) or a bond
XREAPRWDAPLRDPALFQLL

SEQ ID NO: 125 - protein, 20 aa, synthetic construct
  REGION 1..20: note = synthetic
  SITE 1: note = MISC_FEATURE - conjugated to biotin
  SITE 1: note = MISC_FEATURE - X represent the linker (LI) or a bond
XREAPRWDAPLRDPALRYLL

SEQ ID NO: 126 - protein, 20 aa, synthetic construct
  REGION 1..20: note = synthetic
  SITE 1: note = MISC_FEATURE - conjugated to biotin
  SITE 1: note = MISC_FEATURE - X represent the linker (LI) or a bond
XREAPRWDAPLRDPALRQYL

SEQ ID NO: 127 - protein, 20 aa, synthetic construct
  REGION 1..20: note = synthetic
  SITE 1: note = MISC_FEATURE - conjugated to biotin
  SITE 1: note = MISC_FEATURE - X represent the linker (LI) or a bond
XREAPRWDAPLRDPALRYYL

SEQ ID NO: 128 - protein, 13 aa, synthetic construct
  REGION 1..13: note = synthetic
  SITE 1: note = MISC_FEATURE - conjugated to biotin
  SITE 1: note = MISC_FEATURE - X represent the linker (LI) or a bond
XAPLRDPAPRQLL

SEQ ID NO: 129 - protein, 20 aa, synthetic construct
  REGION 1..20: note = synthetic
  SITE 1: note = MISC_FEATURE - conjugated to biotin
  SITE 1: note = MISC_FEATURE - X represent the linker (LI) or a bond
XPYILKRQLYENKPRRPYIL

SEQ ID NO: 130 - protein, 13 aa, synthetic construct
  REGION 1..13: note = synthetic
  SITE 1: note = MISC_FEATURE - conjugated to biotin
  SITE 1: note = MISC_FEATURE - X represent the linker (LI) or a bond
XLYENKPRRPYIL

SEQ ID NO: 131 - protein, 15 aa, synthetic construct
  REGION 1..15: note = synthetic
  PEPTIDE 1..15
GGSGGGGSGGGGSGG

SEQ ID NO: 132 - protein, 7 aa, synthetic construct
  REGION 1..7: note = synthetic
  PEPTIDE 1..7
GGSGGGG

SEQ ID NO: 133 - protein, 17 aa, synthetic construct
  REGION 1..17: note = synthetic
  PEPTIDE 1..17
GGGGSGGGGSGGGGSGG

SEQ ID NO: 134 - protein, 13 aa, synthetic construct
  REGION 1..13: note = synthetic
  PEPTIDE 1..13
GGSGGGGSGGGGS

SEQ ID NO: 135 - protein, 11 aa, synthetic construct
  REGION 1..11: note = synthetic
  PEPTIDE 1..11
GGSGGGGSGGG

SEQ ID NO: 136 - protein, 9 aa, synthetic construct
  REGION 1..9: note = synthetic
  PEPTIDE 1..9
GGSGGGGSG

SEQ ID NO: 137 - protein, 7 aa, synthetic construct
  REGION 1..7: note = synthetic
  PEPTIDE 1..7
GGSGGGG

SEQ ID NO: 138 - protein, 5 aa, synthetic construct
  REGION 1..5: note = synthetic
  PEPTIDE 1..5
GGSGG

SEQ ID NO: 139 - skipped

SEQ ID NO: 140 - protein, 16 aa, synthetic construct
  REGION 1..16: note = synthetic
  PEPTIDE 1..16
CGGSGGGGSGGGGSGG

SEQ ID NO: 141 - protein, 13 aa, synthetic construct
  REGION 1..13: note = synthetic
  SITE 1: note = MISC_FEATURE - conjugated to biotin N-term
GGSGGGGSGRQLL

SEQ ID NO: 142 - protein, 17 aa, synthetic construct
  REGION 1..17: note = synthetic
  SITE 1: note = MISC_FEATURE - conjugated to biotin N-term
GGSGGGGSGGGGSRQLL

SEQ ID NO: 143 - protein, 11 aa, synthetic construct
  REGION 1..11: note = synthetic
  SITE 1: note = MISC_FEATURE - conjugated to biotin N-term
GGSGGGGFQLL

SEQ ID NO: 144 - protein, 9 aa, synthetic construct
  REGION 1..9: note = synthetic
  SITE 1: note = MISC_FEATURE - conjugated to biotin N-term
GGSGGFQLL

SEQ ID NO: 145 - protein, 13 aa, synthetic construct
  REGION 1..13: note = synthetic
  SITE 1: note = MISC_FEATURE - conjugated to biotin N-term
GGSGGGGSGFQLL

SEQ ID NO: 146 - protein, 17 aa, synthetic construct
  REGION 1..17: note = synthetic
  SITE 1: note = MISC_FEATURE - conjugated to biotin N-term
GGSGGGGSGGGGSFQLL

SEQ ID NO: 147 - protein, 19 aa, synthetic construct
  REGION 1..19: note = synthetic
  SITE 1: note = MISC_FEATURE - conjugated to biotin N-term
GGSGGGGSGGGGSGGRQLL

SEQ ID NO: 148 - protein, 15 aa, synthetic construct
  REGION 1..15: note = synthetic
  SITE 1: note = MISC_FEATURE - conjugated to biotin N-term
GGSGGGGSGGGFQLL

SEQ ID NO: 149 - protein, 15 aa, synthetic construct
  REGION 1..15: note = synthetic
  SITE 1: note = MISC_FEATURE - conjugated to biotin N-term
GGSGGGGSGGGRQLL

SEQ ID NO: 150 - protein, 11 aa, synthetic construct
  REGION 1..11: note = synthetic
  SITE 1: note = MISC_FEATURE - conjugated to biotin N-term
GGSGGGGRQLL

SEQ ID NO: 151 - protein, 9 aa, synthetic construct
  REGION 1..9: note = synthetic
  SITE 1: note = MISC_FEATURE - conjugated to biotin N-term
GGSGGRQLL

SEQ ID NO: 152 - protein, 7 aa, synthetic construct
  REGION 1..7: note = synthetic
  SITE 1: note = MISC_FEATURE - conjugated to biotin N-term
GGSRQLL

SEQ ID NO: 153 - protein, 6 aa, synthetic construct
  REGION 1..6: note = synthetic
  SITE 1: note = MISC_FEATURE - conjugated to biotin N-term
GGRQLL

SEQ ID NO: 154 - protein, 4 aa, synthetic construct
  REGION 1..4: note = synthetic
  SITE 1: note = MISC_FEATURE - conjugated to biotin N-term
RQLL

SEQ ID NO: 155 - protein, 19 aa, synthetic construct
  REGION 1..19: note = synthetic
  SITE 1: note = MISC_FEATURE - conjugated to biotin N-term
GGSGGGGSGGGGSGGFQLL

SEQ ID NO: 156 - protein, 15 aa, synthetic construct
  REGION 1..15: note = synthetic
  SITE 1: note = MISC_FEATURE - conjugated to biotin N-term
GGSGGGGSGGGGSGG

SEQ ID NO: 157 - protein, 19 aa, synthetic construct
  REGION 1..19: note = synthetic
  SITE 1: note = MISC_FEATURE - conjugated to biotin N-term
  SITE 19: note = MISC_FEATURE - amidated C-term
GGSGGGGSGGGGSGGRQLL

SEQ ID NO: 158 - protein, 158 aa, synthetic construct
  REGION 1..158: note = synthetic
  REGION 1..158: note = MISC_FEATURE - Nanobody-linker-RQLL
MGGTHHHHHHENLYFQGQVQLQESGGGLVQPGGSLRLSCAASGRTISRYAMSWFQAPGKEREFVAVARRSGDGAFYADSVQGRFTVSRDDAKNTVYLQMNSLKPEDTAVYCAIDSDTFYSGSYDYWGQGTQVTVSSEGGGGSGGGGSGGGGSGGRQLL

SEQ ID NO: 159 - protein, 20 aa, synthetic construct
  REGION 1..20: note = synthetic
  REGION 1..20: note = MISC_FEATURE - refers to antibody conjugate of alirocumab with this sequence. (Alirocumab-linker-RQLL)
CGGSGGGGSGGGGSGGRQLL

SEQ ID NO: 160 - protein, 16 aa, synthetic construct
  REGION 1..16: note = synthetic
  REGION 1..16: note = MISC_FEATURE - refers to antibody conjugate of alirocumab with this sequence (Alirocumab-linker)
CGGSGGGGSGGGGSGG

SEQ ID NO: 161 - protein, 20 aa, synthetic construct
  REGION 1..20: note = synthetic
  REGION 1..20: note = MISC_FEATURE - refers to antibody conjugate of Adalimumab with this sequence. (Adalimumab-linker-RQLL)
CGGSGGGGSGGGGSGGRQLL

SEQ ID NO: 162 - protein, 16 aa, synthetic construct
  REGION 1..16: note = synthetic
  REGION 1..16: note = MISC_FEATURE - refers to antibody conjugate of Adalimumab with this sequence (Adalimumab-linker)
CGGSGGGGSGGGGSGG

SEQ ID NO: 163 - protein, 4 aa, synthetic construct
  REGION 1..4: note = synthetic
  PEPTIDE 1..4
RQLI

SEQ ID NO: 164 - protein, 4 aa, synthetic construct
  REGION 1..4: note = synthetic
  PEPTIDE 1..4
RQLF

SEQ ID NO: 165 - protein, 4 aa, synthetic construct
  REGION 1..4: note = synthetic
  PEPTIDE 1..4
RQLM

SEQ ID NO: 166 - skipped

SEQ ID NO: 167 - protein, 19 aa, synthetic construct
  REGION 1..19: note = synthetic
  PEPTIDE 1..19
GGSGGGGSGGGGSGGRQLL

SEQ ID NO: 168 - protein, 20 aa, synthetic construct
  REGION 1..20: note = synthetic
  SITE 1: note = MISC_FEATURE - conjugated to NHS
CGGSGGGGSGGGGSGGRQLL

SEQ ID NO: 169 - protein, 16 aa, synthetic construct
  REGION 1..16: note = synthetic
  SITE 1: note = MISC_FEATURE - conjugated to NHS
CGGSGGGGSGGGGSGG

SEQ ID NO: 170 - protein, 692 aa, synthetic construct
  REGION 1..692: note = synthetic
  REGION 1..692: note = MISC_FEATURE - PCSK-9
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQ

SEQ ID NO: 171 - protein, 698 aa, synthetic construct
  REGION 1..698: note = synthetic
  REGION 1..698: note = MISC_FEATURE - PCSK-9-6his
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQHHHHHH

SEQ ID NO: 172 - protein, 32 aa, synthetic construct
  REGION 1..32: note = synthetic
  PEPTIDE 1..32
TVFTSWEEYLDWVGGSGGGGSGGGGSGGRQLL

SEQ ID NO: 173 - protein, 20 aa, synthetic construct
  REGION 1..20: note = synthetic
CGGSGGGGSGGGGSGGRQLL

SEQ ID NO: 174 - DNA, 6111 bp, synthetic construct
  misc_feature 1..6111: note = plasmid
  misc_feature 1..6111: note = plasmid for Nanobody-link
  source 1..6111: mol_type = other DNA
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggtggtacccaccaccatcaccaccatgaaaatctatatttccagggtcaggttcaactgcaggagagcggcggcggcctggtgcagccgggtggctctttacgtctgagctgtgcggcaagcggtagaactattagccgctacgctatgagctggtttcgtcaagcaccgggtaaagaacgtgagttcgttgcggtggcccgtcgttcgggtgatggtgcgttttacgcggatagcgttcagggtcgctttaccgtaagccgtgacgacgcgaagaacaccgtgtatctgcaaatgaatagcttgaagccggaagataccgctgtgtactactgcgccatcgactcggataccttctacagcggttcctatgactattggggtcaaggtacgcaggtcaccgtttcctccgagggtggcgggggctctggcggcggcggttccggcggcggtggctctggtggttaaggatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 175 - DNA, 6123 bp, synthetic construct
  misc_feature 1..6123: note = plasmid
  misc_feature 1..6123: note = plasmid for nanobody-linker-RQLL
  source 1..6123: mol_type = other DNA
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcggaacccaccaccatcaccaccatgaaaatctatatttccagggtcaggttcaactgcaggagagcggcggcggcctggtgcagccgggtggctctttacgtctgagctgtgcggcaagcggtagaactattagccgctacgctatgagctggtttcgtcaagcaccgggtaaagaacgtgagttcgttgcggtggcccgtcgttcgggtgatggtgcgttttacgcggatagcgttcagggtcgctttaccgtaagccgtgacgacgcgaagaacaccgtgtatctgcaaatgaatagcttgaagccggaagataccgctgtgtactactgcgccatcgactcggataccttctacagcggttcctatgactattggggtcaaggtacgcaggtcaccgtttcctccgagggtggcgggggctctggcggcggcggttccggcggcggtggctctggtggtcgccaattgctgtaaggatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 176 - protein, 4563 aa, Homo sapiens
  REGION 1..4563: note = MISC_FEATURE - Apolipoprotein B
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPQTDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSYDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVLENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDILEHVKHFVINLIGDFEVAEKINAFRAKVHELIERYEVDQQIQVLMDKLVELAHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMLNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPDFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVATTTKAQIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFIIPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTFAHRDFSAEYEEDGKYEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQEFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFSDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 177 - protein, 247 aa, Homo sapiens
  REGION 1..247: note = MISC_FEATURE - BDNF
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 178 - protein, 241 aa, Homo sapiens
  REGION 1..241: note = MISC_FEATURE - NGF
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 179 - protein, 21 aa, synthetic construct
WSGPIGVSWGLRAAAAGFQLL

SEQ ID NO: 180 - protein, 17 aa, synthetic construct
WSGPIGVSWGLRAAAAG

SEQ ID NO: 181 - protein, 16 aa, synthetic construct
CREAPRWDAPLRDPAL

SEQ ID NO: 182 - protein, 21 aa, synthetic construct
WSGPIGVSWGLRAAAAGFQLL

SEQ ID NO: 183 - protein, 21 aa, synthetic construct
WSGPIGVSWGLRAAAAGRQLL

SEQ ID NO: 184 - protein, 20 aa, synthetic construct
CREAPRWDAPLRDPALRQLL

SEQ ID NO: 185 - protein, 20 aa, synthetic construct
  MOD_RES 20: note = AMIDATION
CREAPRWDAPLRDPALRQLL

SEQ ID NO: 186 - protein, 20 aa, synthetic construct
CREAPRWDAPLRDPALFQLL

SEQ ID NO: 187 - protein, 20 aa, synthetic construct
  MOD_RES 20: note = AMIDATION
CREAPRWDAPLRDPALFQLL

SEQ ID NO: 188 - protein, 19 aa, synthetic construct
  MOD_RES 19: note = AMIDATION
  SITE 1: note = conjugated to Biotin at N-term
REAPRWDAPLRDPALFQLL

SEQ ID NO: 189 - protein, 19 aa, synthetic construct
  SITE 1: note = conjugated to biotion at N-term
REAPRWDAPLRDPALFQLL

SEQ ID NO: 190 - protein, 19 aa, synthetic construct
  SITE 1: note = conjugated to Biotin at N-term
  MOD_RES 19: note = AMIDATION
REAPRWDAPLRDPALRQLL

SEQ ID NO: 191 - protein, 19 aa, synthetic construct
REAPRWDAPLRDPALRQLL

SEQ ID NO: 192 - protein, 20 aa, synthetic construct
  SITE 1: note = Conjugated to adalimumab via N-term
CREAPRWDAPLRDPALRQLL

SEQ ID NO: 193 - protein, 20 aa, synthetic construct
  MOD_RES 20: note = AMIDATION
  SITE 1: note = Conjugated to adalimumab via N-term
CREAPRWDAPLRDPALRQLL

SEQ ID NO: 194 - protein, 20 aa, synthetic construct
  SITE 1: note = Conjugated to adalimumab via N-term
CREAPRWDAPLRDPALFQLL

SEQ ID NO: 195 - protein, 20 aa, synthetic construct
  MOD_RES 20: note = AMIDATION
  SITE 1: note = Conjugated to adalimumab via N-term
CREAPRWDAPLRDPALFQLL

SEQ ID NO: 196 - protein, 4 aa, synthetic construct
AARL

SEQ ID NO: 197 - protein, 5 aa, synthetic construct
PIPLV